(19) United States
(12) Patent Application Publication
(10) Pub. No.: US 2018/0073006 A1
(43) Pub. Date: Mar. 15, 2018

(54) NOVEL METALLOPROTEASES

(71) Applicant: DANISCO US INC., Palo Alto, CA (US)

(72) Inventors: Lilia M. Babe, Emerald Hills, CA (US); Richard R. Bott, Burlingame, CA (US); Roopa Ghirnikar, Sunnyvale, CA (US); Frits Goedegebuur, Vlaardingen (NL); Xiaogang Gu, Shanghai (CN); Marc Kolkman, Oegsteest (NL); Jian Yao, Sunnyvale, CA (US); Shukun Yu, Bunkeflostrand (SE)

(21) Appl. No.: 15/725,627

(22) Filed: Oct. 5, 2017

Related U.S. Application Data
(63) Continuation of application No. 14/893,440, filed on Nov. 23, 2015, now abandoned.
(60) Provisional application No. 61/874,813, filed on Sep. 6, 2013.

(30) Foreign Application Priority Data
May 29, 2013 (CN) PCT/CN2013/076369
May 29, 2013 (CN) PCT/CN2013/076383
May 29, 2013 (CN) PCT/CN2013/076384
May 29, 2013 (CN) PCT/CN2013/076386
May 29, 2013 (CN) PCT/CN2013/076387
May 29, 2013 (CN) PCT/CN2013/076390
May 29, 2013 (CN) PCT/CN2013/076398
May 29, 2013 (CN) PCT/CN2013/076401
May 29, 2013 (CN) PCT/CN2013/076406
May 29, 2013 (CN) PCT/CN2013/076414
May 29, 2013 (CN) PCT/CN2013/076415
May 29, 2013 (CN) PCT/CN2013/076419

(51) Int. Cl. C12N 9/54; C11D 3/386

(52) U.S. Cl. CPC C12N 9/54; C11D 3/386; C12Y 304/24027; C12Y 304/24

(57) ABSTRACT

Aspects of the present compositions and methods relate to novel metalloproteases polynucleotides encoding the novel metalloprotease, compositions and methods for use thereof.

### CROSS REFERENCE TO RELATED APPLICATIONS

This application is a Continuation of Ser. No. 14/893,440, filed Nov. 23, 2015, which is a 371 of National Phase Application of PCT/US2014/040063, filed on May 29, 2014, which claims benefit of priority from International patent applications Serial No. PCT/CN2013/076419; Serial No. PCT/CN2013/076387; Serial No. PCT/CN2013/076401; Serial No. PCT/CN2013/076406; Serial No. PCT/CN2013/076414; PCT/CN2013/076384; Serial No. PCT/CN2013/076398; PCT/CN2013/076415; Serial No. PCT/CN2013/076386; Serial No. PCT/CN2013/076390; PCT/CN2013/076383; Serial No. PCT/CN2013/076369, all filed on 29 May 2013, and also claims benefit of priority from U.S. Provisional Application Ser. No. 61/874,813 filed on 6 Sep. 2013, the contents of all of which are incorporated herein by reference in their entirety.

### SEQUENCE LISTING

The sequence listing submitted via EFS, in compliance with 37 C.F.R. § 1.52(e), is incorporated herein by reference. The sequence listing text file submitted via EFS contains the file “20171005_NB40426USCNT_SeqLst_ST25.txt”, created on Oct. 4, 2017, which is 49,152 bytes in size.

### FIELD OF THE INVENTION

The present disclosure relates to proteases. Compositions containing the proteases are suitable for use in cleaning, food and feed as well as in a variety of other industrial applications.

### BACKGROUND

Metalloproteases (MPs) are among the hydrolases that mediate nucleophilic attack on peptide bonds using a water molecule coordinated in the active site. Thermolysin-like proteases are found in the M4 family as defined by MEROPS (Rawlings et al., (2012) Nucleic Acids Res 40:D343-D350). They are generally active at elevated temperatures and this stability is attributed to calcium binding. Although proteases have long been known in the art of industrial enzymes, there remains a need for novel proteases that are suitable for particular conditions and uses, such as environments that have calcium chelators that destabilize metalloproteases.

### SUMMARY

The present disclosure provides, inter alia, metalloproteases comprising modifications in calcium binding regions, novel metalloprotease polypeptides with signature amino acids in the calcium binding regions, nucleic acids encoding the same, and compositions and methods related to the production and use thereof.

In some embodiments, the invention is a metalloprotease polypeptide comprising one or more calcium binding regions. In some embodiments, the polypeptide comprises a modification in at least one amino acid residue in one of the calcium binding regions, Ca1-2, Ca3 and Ca4, (including residues 55-66, 136, 138, 177-190, and 193-200) of the polypeptide, wherein the amino acid positions of the polypeptide are numbered by correspondence with the amino acid sequence of Bacillus thermoproteolyticus metalloprotease set forth in SEQ ID NO: 13. In some embodiments, the polypeptide has a calcium binding region Ca1-2 that binds fewer than two calcium ions. In some embodiments, the polypeptide has a calcium binding region 3 that binds fewer than one calcium ion. In some embodiments, the polypeptide has a calcium binding region 4 that binds fewer than one calcium ion. In some embodiments, the polypeptide is a variant of a parent polypeptide. In some embodiments, the parent polypeptide is an M4 metalloprotease. In some embodiments, the polypeptide has at least 60% sequence identity to any one of SEQ ID NOs: 1-15.

In some embodiments, the invention is a composition comprising at least one variant as listed above. In some embodiments, the invention is a method of cleaning using a cleaning composition as listed above.

### BRIEF DESCRIPTION OF THE DRAWINGS

FIG. 1 provides a schematic showing the arrangement of the two independent molecules of PehPro1 in the asymmetric unit of the crystal unit cell.

FIG. 2 provides a stereo showing a schematic of the Thermolysin main chain folding (black) and the schematic of PehPro1 (light gray).

FIG. 3 provides a close up view of the 3 residue deletion.

FIG. 4 provides a close up view of the 5 residue deletion.

FIG. 5 provides a comparison of calcium binding site 1 in PehPro1 and the Ca1-2 calcium binding site in Thermolysin.

FIG. 6 provides a schematic comparing Thermolysin (black lines) with PehPro1 (light gray sticks) in the vicinity of the second calcium binding site (Ca4) in PehPro1.

FIG. 7 provides a comparison of the structures of Thermolysin (black lines) and PehPro1 (light gray sticks) in the region of calcium binding Ca3 present in Thermolysin.

FIG. 8 provides a schematic comparing the main chain folding of PehPro1 (dark gray) and PpoPro2 (light gray).

FIG. 9 provides a comparison of the first common calcium binding site between PehPro1 (light gray) and Ppopro2 (dark gray).

FIG. 10 provides a comparison of the structure of PehPro1 (light gray) and PpoPro2 (dark gray) at the second common calcium site.

FIG. 11 provides a comparison of the third calcium binding site seen in PpoPro2 (dark gray) as compared to PehPro1 (light gray).

FIG. 12a-f provides a structure-based alignment of the various metalloproteases Peh1.A (Paenibacillus ehimensis, protein 1)(SEQ ID NO: 1), PbaPro1 (Paenibacillus barcinonensis, protein 1) (SEQ ID NO: 2), PhuPro1 (Paenibacillus hunanensis, protein 1) (SEQ ID NO: 3), PpoPro2 (Paenibacillus polymyxa, protein 2) (SEQ ID NO: 4), PpoPro1 (Paenibacillus polymyxa, protein 1) (SEQ ID NO: 5), PamPro1 (Paenibacillus amylolyticus, protein 1)(SEQ ID NO: 6), PhuPro2 (Paenibacillus hunanensis, protein 2) (SEQ ID NO: 7), PspPro2 (Paenibacillus sp., protein 2) (SEQ ID NO: 8), PspPro3 (Paenibacillus sp., protein 3) (SEQ ID NO: 9), PpePro1 (Paenibacillus peoriae, protein 1) (SEQ ID NO: 10), PtePro1 (Paenibacillus terrae, protein 1) (SEQ ID NO: 11), BbrPro1 (Brevibacillus brevis, protein 1) (SEQ ID NO: 12), 1KEI.A (Bacillus thermoproteolyticus, thermolysin) (SEQ ID NO: 13), 1NPC.A (Bacillus cereus) (SEQ ID NO: 14), Npre_var (Bacillus subtilis) (SEQ ID NO: 15).

FIG. 13 provides a comparison of the calcium binding sites of NprE (gray sticks) with the double calcium site (Ca1,2) of Thermolysin (black lines).

FIG. 14 provides a comparison of NprE variant with Thermolysin at the Ca3 site.

FIG. 15 provides several proteases contain the double delete and lack of the D×D motif shown in a rectangle.

FIG. 16 provides a stereodiagram comparing the overall main chain folding pattern of Thermolysin (black) with the NprE variant structure (gray).

FIG. 17 provides a comparison of the structures of the NprE variant and Thermolysin at the Ca4 binding site of Thermolysin. The deletion of three residues in NprE relative to Thermolysin results in the elimination of a calcium binding site.

### DETAILED DESCRIPTION

The present invention provides novel variant metalloproteases having modifications at calcium binding regions. The MEROPS database (http://merops.sanger.ac.uk) groups peptidases into families based on sequence homology [Rawlings et al. (2010) Nucleic Acid Res. 38:D227]. A peptidase is classified into a family based on sharing significant similarities in amino acid sequence with the type example or another member of the family. In the current release (Release 9.4) of the MEROPS database, there are a total of 63 metalloprotease families, nine of which include but are not limited to BEMP members. These proteases are distributed among 9 families of metalloproteases because of differences in primary sequences and structural characteristics. So far, all BEMPs are endoproteases that harbor 1 catalytic Zinc ion in their active center. They are synthesized as inactive zymogens with a propeptide and their main function is nutrition of the microorganism. Bacterial extracellular metalloproteases (BEMPs) are a large group of metal-containing proteases secreted by heterotropic bacteria [Wu and Chen (2011) Appl. Biol. Biotechnol. 92:253]. BEMPs are distributed among the metalloprotease families M4, M5, M9, M10, M12, M13, M23, M30, and M34. The M4 is a large family of metalloproteases, mostly BEMPs. Thermolysin is the prototype of the M4 family.

Thermolysin-like proteases are broad-specificity proteases which contain a catalytic zinc ion in their active sites. The thermostable Bacillus neutral metalloproteases bind 4 Ca²⁺ ions. Two Ca²⁺ ions are bound in one double calcium binding site (Ca1-2) and 2 Ca²⁺ ions are bound in single binding sites Ca3 and Ca4 [Stark et al (1992) Eur. J. Biochem. 207:207, Veltman et al (1998) Biochem. 37:5312]. Several studies have shown that these proteases are dependent on calcium binding for their stability [Veltman et al (1997) FEBS 405:241]. The single sites Ca3 and Ca4 are absent in the thermolysin-like proteases considered thermolabile [Eijsink et al (2011) Prot. Sci. 20:1346].

It has also become known that thermolysin-like proteinases can perform well in a number of industrial applications such as a detergent additive for laundry and dish cleaning, potentially as feed additives, fermentation aides, as well as a number of pharmaceutical application such as cell culture and tissue dissociation. Earlier studies have demonstrated the importance of calcium binding in a number of mutational studies. In Eijsink et al 2011, [Eijsink, Matthews and Vriend (2011) Prot Sci 20:1346-1355], mutation of Asp57 or Asp 59 in the Ca3 site were shown to dramatically reduce stability in thermolysin. In the same paper the authors postulate that calcium binding site may have evolved evolutionarily as a means or regulating function but destabilizing structure and hence function in the low calcium environment of the cytosol until secreted into a higher calcium environment outside the cell membrane.

Metalloproteases, for example, M4 clan metalloproteases, have calcium binding regions. Without being bound by theory, these calcium binding regions are thought to contribute to the thermostability of these molecules. In some applications, it is beneficial to reduce the dependence of the metalloproteases on calcium binding. For example, detergent compositions contain metal chelators, such as surfactants, which compete for calcium ions and affects the amount of free calcium available to bind the enzyme [Stoner et al. (2005) Biotechnol Prog. 21(6): 1716-23]. In detergent environments, metalloproteases can be subject to destabilization and autolysis due to this lack of free calcium. Thus, there is a need in the art to discover improved metalloproteases which are stable in environments that compete for free calcium, such as detergent compositions, while allowing for maintained proteolytic activity of the metalloproteases in these environments. As such, it would be beneficial to find variant metalloproteases with modified calcium binding regions.

The invention described here arises in part from the observation of two novel crystal structures of thermolysin-like proteases having fewer calcium binding sites (PehPro1 as shown in Example 2, and PpoPro2, Paenibacillus polymyxa protease in Ruf et al 2013 [Ruf et al (2013) Acta Cryst. D69:24-31). The elimination of calcium binding is attributed to a combination of specific amino acid substitutions and deletion that are found to be common to these and related structures resulting in fewer calcium binding coordination sites. Based on these finding, a means for further reducing the number of calcium bound to only one or none is proposed for thermolysin and other thermolysin-like proteinases.

In some embodiments, the invention is variant metalloproteases having modified calcium binding regions. In some embodiments, these modified calcium binding regions result in reduced calcium binding of the metalloprotease enzyme. In other embodiments, the invention is novel metalloproteases having newly discovered calcium binding regions. In some embodiments, the invention includes compositions comprising at least one of the novel metalloprotease enzymes set forth herein. Some such compositions comprise detergent compositions. The metalloprotease enzymes of the present invention can be combined with other enzymes useful in detergent compositions. The invention also provides methods of cleaning using metalloprotease enzymes of the present invention.

### Definitions and Abbreviations

Unless otherwise indicated, the practice of the present invention involves conventional techniques commonly used in molecular biology, protein engineering, microbiology, and recombinant DNA technology, which are within the skill of the art. Such techniques are known to those of skill in the art and are described in numerous texts and reference works well known to those of skill in the art. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this invention pertains. Many technical dictionaries are known to those of skill in the art. Although any methods and materials similar or equivalent to those described herein find use in the practice of the present invention, some suitable methods and materials are described herein. Accordingly, the terms defined immediately below are more fully described by reference to the Specification as a whole. Also, as used herein, the singular “a”, “an” and “the” includes the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Furthermore, the headings provided herein are not limitations of the various aspects or embodiments of the invention.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the terms “protease” and “proteinase” refer to an enzyme that has the ability to break down proteins and peptides. A protease has the ability to conduct “proteolysis,” by hydrolysis of peptide bonds that link amino acids together in a peptide or polypeptide chain forming the protein. This activity of a protease as a protein-digesting enzyme is referred to as “proteolytic activity.” Many well known procedures exist for measuring proteolytic activity (See e.g., Kalisz, “Microbial Proteinases,” In: Fiechter (ed.), Advances in Biochemical Engineering/Biotechnology, (1988)). For example, proteolytic activity may be ascertained by comparative assays which analyze the respective protease's ability to hydrolyze a suitable substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO 99/34011 and U.S. Pat. No. 6,376,450, both of which are incorporated herein by reference). The pNA peptidyl assay (See e.g., Del Mar et al., Anal. Biochem. 99:316-320 [1979]) also finds use in determining the active enzyme concentration. This assay measures the rate at which p-nitroaniline is released as the enzyme hydrolyzes a soluble synthetic substrate, such as succinyl-alanine-alanine-proline-phenylalanine-p-nitroanilide (suc-AAPF-pNA)(SEQ ID NO: 24). The rate of production of yellow color from the hydrolysis reaction is measured at 410 nm on a spectrophotometer and is proportional to the active enzyme concentration. In addition, absorbance measurements at 280 nanometers (nm) can be used to determine the total protein concentration in a sample of purified protein. The activity on substrate/protein concentration gives the enzyme specific activity.

As used herein, the term “variant polypeptide” refers to a polypeptide comprising an amino acid sequence that differs in at least one amino acid residue from the amino acid sequence of a parent or reference polypeptide (including but not limited to wild-type polypeptides).

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, “calcium binding site” refers to a region within a metalloprotease which can bind a calcium ion in the presence of free calcium. Calcium can act to assist in maintaining the structural integrity of metalloproteases under many conditions. In some embodiments, the amount of free calcium can be related to the water hardness during wash conditions, and can range from soft water, having less than 1.0 Calcium grains per gallon, to slightly hard water, having from about 1.0 to 3.5 Calcium grains per gallon, to moderately hard water, having from about 3.5 to 7.0 Calcium grains per gallon, to hard water, having from about 7.0 to 10.5 or more Calcium grains per gallon. In some embodiments of the present invention, the characteristics of the calcium binding site are modified compared to a parent or reference metalloprotease so as to modify the performance of the metalloprotease. Modification of the calcium binding site may include reducing or increasing the affinity of the site to bind calcium ion. Modifying the performance of the metalloprotease is intended to include modification of the stability (e.g., oxidative or thermal) or the activity (e.g., the rate or efficiency with which the metalloprotease hydrolyzes a protein substrate) of the enzyme in its various applications.

As used herein, “calcium ligand” means an amino acid residue or residues within a metalloprotease enzyme which forms a ligand with calcium ion bound within a calcium binding site.

The terms “polynucleotide” and “nucleic acid,” which are used interchangeably herein, refer to a polymer of any length of nucleotide monomers covalently bonded in a chain. DNA (deoxyribonucleic acid), a polynucleotide comprising deoxyribonucleotides, and RNA (ribonucleic acid), a polymer of ribonucleotides, are examples of polynucleotides or nucleic acids having distinct biological function. Polynucleotides or nucleic acids include, but are not limited to, a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The following are non-limiting examples of polynucleotides: genes, gene fragments, chromosomal fragments, expressed sequence tag(s) (EST(s)), exons, introns, messenger RNA (mRNA), transfer RNA (tRNA), ribosomal RNA (rRNA), ribozymes, complementary DNA (cDNA), recombinant polynucleotides, branched polynucleotides, plasmids, vectors, isolated DNA of any sequence, isolated RNA of any sequence, nucleic acid probes, and primers.

As used herein, the term “modification” refers to changes made to a reference amino acid or nucleic acid sequence. It is intended that the term encompass substitutions, insertions and deletions.

As used herein, the term “vector” refers to a nucleic acid construct used to introduce or transfer nucleic acid(s) into a target cell or tissue. A vector is typically used to introduce foreign DNA into a cell or tissue. Vectors include plasmids, cloning vectors, bacteriophages, viruses (e.g., viral vector), cosmids, expression vectors, shuttle vectors, and the like. A vector typically includes an origin of replication, a multicloning site, and a selectable marker. The process of inserting a vector into a target cell is typically referred to as transformation. The present invention includes, in some embodiments, a vector that comprises a DNA sequence encoding a metalloprotease polypeptide (e.g., precursor or mature metalloprotease polypeptide) that is operably linked to a suitable prosequence (e.g., secretory, signal peptide sequence, etc.) capable of effecting the expression of the DNA sequence in a suitable host, and the folding and translocation of the recombinant polypeptide chain.

As used herein, the term “expression cassette,” “expression plasmid” or “expression vector” refers to a nucleic acid construct or vector generated recombinantly or synthetically for the expression of a nucleic acid of interest in a target cell. An expression vector or expression cassette typically comprises a promoter nucleotide sequence that drives expression of the foreign nucleic acid. The expression vector or cassette also typically includes any other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Many prokaryotic and eukaryotic expression vectors are commercially available.

In some embodiments, the ends of the sequence are closed such that the DNA construct forms a closed circle. The nucleic acid sequence of interest, which is incorporated into the DNA construct, using techniques well known in the art, may be a wild-type, mutant, or modified nucleic acid. In some embodiments, the DNA construct comprises one or more nucleic acid sequences homologous to the host cell chromosome. In other embodiments, the DNA construct comprises one or more non-homologous nucleotide sequences. Once the DNA construct is assembled in vitro, it may be used, for example, to: 1) insert heterologous sequences into a desired target sequence of a host cell; and/or 2) mutagenize a region of the host cell chromosome (i.e., replace an endogenous sequence with a heterologous sequence); 3) delete target genes; and/or 4) introduce a replicating plasmid into the host. “DNA construct” is used interchangeably herein with “expression cassette.”

As used herein, a “plasmid” refers to an extrachromosomal DNA molecule which is capable of replicating independently from the chromosomal DNA. A plasmid is double stranded (ds) and may be circular and is typically used as a cloning vector.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction (See e.g., Ferrari et al., “Genetics,” in Hardwood et al. (eds.), Bacillus, Plenum Publishing Corp., pp. 57-72 [1989]).

Transformation refers to the genetic alteration of a cell which results from the uptake, optional genomic incorporation, and expression of genetic material (e.g., DNA).

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

As used herein the term “gene” refers to a polynucleotide (e.g., a DNA segment), that encodes a polypeptide and includes regions preceding and following the coding regions as well as intervening sequences (introns) between individual coding segments (exons).

As used herein, “recombinant” when used with reference to a cell typically indicates that the cell has been modified by the introduction of a foreign nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) but which has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro, and transfer of the recombinant DNA into cells where it may be expressed or propagated, thereby producing a recombinant polypeptide. “Recombination,” “recombining,” and “recombined” of polynucleotides or nucleic acids refer generally to the assembly or combining of two or more nucleic acid or polynucleotide strands or fragments to generate a new polynucleotide or nucleic acid. The recombinant polynucleotide or nucleic acid is sometimes referred to as a chimera. A nucleic acid or polypeptide is “recombinant” when it is artificial or engineered.

A nucleic acid or polynucleotide is said to “encode” a polypeptide if, in its native state or when manipulated by methods known to those of skill in the art, it can be transcribed and/or translated to produce the polypeptide or a fragment thereof. The anti-sense strand of such a nucleic acid is also said to encode the sequence.

“Host strain” or “host cell” refers to a suitable host for an expression vector comprising a DNA sequence of interest.

A “protein” or “polypeptide” comprises a polymeric sequence of amino acid residues. The terms “protein” and “polypeptide” are used interchangeably herein. The single and 3-letter code for amino acids as defined in conformity with the IUPAC-IUB Joint Commission on Biochemical Nomenclature (JCBN) is used through out this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. Mutations can also be named by using the three letter code for an amino acid followed by its position in the polypeptide chain as counted from the N-terminus; for example, Ala10 for alanine at position 10. Multiple mutations are indicated by inserting a “−,” “+,” “/,” or “;” between the mutations. Mutations at positions 87 and 90 are represented as either “G087S-A090Y” or “G87S-A90Y” or “G87S+A90Y” or “G087S+A090Y”. For insertions, one or more inserted amino acids can be listed after a position. For example, “G087GS” describes a serine inserted after the glycine at position 87; as a second example, “G087GSA” describes a serine and alanine inserted after the glycine at position 87. Insertions can be done in combination with substitutions; thus, “G087RS” describes a substitution at position 87 from glycine to arginine, followed by an inserted serine residue. For deletions, either a “Δ” or “del” is used following the position number. Thus, for example, “G087del” describes deletion of the glycine at position 87. When describing modifications, a position followed by amino acids listed in parentheses indicates a list of substitutions at that position by any of the listed amino acids. For example, 6(L,I) means position 6 can be substituted with a leucine or isoleucine.

A “prosequence” or “propetide sequence” refers to an amino acid sequence between the signal peptide sequence and mature protease sequence that is necessary for the proper folding and secretion of the protease; they are sometimes referred to as intramolecular chaperones. Cleavage of the prosequence or propeptide sequence results in a mature active protease. Bacterial metalloproteases are often expressed as pro-enzymes.

The term “signal sequence” or “signal peptide” refers to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or peptide without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wild-type” in reference to an amino acid sequence or nucleic acid sequence indicates that the amino acid sequence or nucleic acid sequence is native or naturally occurring sequence. As used herein, the term “naturally-occurring” refers to anything (e.g., proteins, amino acids, or nucleic acid sequences) that are found in nature.

As used herein, the term “non-naturally occurring” refers to anything that is not found in nature (e.g., recombinant nucleic acids and protein sequences produced in the laboratory), as modification of the wild-type sequence.

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related proteins or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question. To exemplify, “proteases derived from Bacillus” refers to those enzymes having proteolytic activity which are naturally produced by Bacillus, as well as to metalloproteases like those produced by Bacillus sources but which through the use of genetic engineering techniques are produced by non-Bacillus organisms transformed with a nucleic acid encoding the serine proteases.

The term “identical” in the context of two nucleic acids or polypeptide sequences refers to the residues in the two sequences that are the same when aligned for maximum correspondence, as measured using one of the following sequence comparison or analysis algorithms.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes). As used herein, “homologous proteins” refers to proteins from different, but usually related species, which are very similar to each other.

As used herein, “% identity or percent identity” refers to sequence identity, at the gene or protein level. The output for these calculations are highly dependent on the algorithm used and the parameters selected such as length of compared sequences. Percent identity may be determined using standard techniques known in the art (See e.g., Smith and Waterman, Adv. Appl. Math. 2:482 [1981]; Needleman and Wunsch, J. Mol. Biol. 48:443 [1970]; Pearson and Lipman, Proc. Natl. Acad. Sci. USA 85:2444 [1988]; software programs such as GAP, BESTFIT, FASTA, and TFASTA in the Wisconsin Genetics Software Package (Genetics Computer Group, Madison, Wis.); and Devereux et al., Nucl. Acid Res. 12:387-395 [1984]). One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (See, Feng and Doolittle, J. Mol. Evol. 35:351-360 [1987]). The method is similar to that described by Higgins and Sharp (See, Higgins and Sharp, CABIOS 5:151-153 [1989]). Useful PILEUP parameters include a default gap weight of 3.00, a default gap length weight of 0.10, and weighted end gaps. Other useful algorithm is the BLAST algorithms described by Altschul et al., (See, Altschul et al., J. Mol. Biol. 215:403-410 [1990]; and Karlin and Altschul, Proc. Natl. Acad. Sci. USA 90:5873-5787 [1993]). The BLAST program uses several search parameters, most of which are set to the default values.

The NCBI BLAST algorithm finds the most relevant sequences in terms of biological similarity but is not recommended for query sequences of less than 20 residues (Altschul, S F et al. (1997) Nucleic Acids Res. 25:3389-3402 and Schaffer, A A et al. (2001) Nucleic Acids Res. 29:2994-3005). Example default BLAST parameters for a nucleic acid sequence searches are:
Neighboring words threshold: 11
E-value cutoff: 10
Scoring Matrix: NUC.3.1 (match=1, mismatch=−3)
Gap Opening: 5
Gap Extension: 2
   and the following parameters for amino acid sequence searches:
Word size: 3
E-value cutoff: 10
Scoring Matrix: BLOSUM62
Gap Opening: 11
Gap extension: 1

A percent (%) amino acid sequence identity value is determined by the number of matching identical residues divided by the total number of residues of the “reference” sequence including any gaps created by the program for optimal/maximum alignment. If a sequence is 90% identical to SEQ ID NO: A, SEQ ID NO: A is the “reference” sequence. BLAST algorithms refer the “reference” sequence as “query” sequence.

The CLUSTAL W algorithm is another example of a sequence alignment algorithm. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

In CLUSTAL algorithms, deletions occurring at either terminus are included. For example, a variant with five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

A polypeptide of interest may be said to be “substantially identical” to a reference polypeptide if the polypeptide of interest comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

A nucleic acid of interest may be said to be “substantially identical” to a reference nucleic acid if the nucleic acid of interest comprises a nucleotide sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the nucleotide sequence of the reference nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

A nucleic acid or polynucleotide is “isolated” when it is at least partially or completely separated from other components, including but not limited to for example, other proteins, nucleic acids, cells, etc. Similarly, a polypeptide, protein or peptide is “isolated” when it is at least partially or completely separated from other components, including but not limited to for example, other proteins, nucleic acids, cells, etc. On a molar basis, an isolated species is more abundant than are other species in a composition. For example, an isolated species may comprise at least about 60%, about 65%, about 70%, about 75%, about 80%, about 85%, about 90%, about 91%, about 92%, about 93%, about 94%, about 95%, about 96%, about 9′7%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

“Hybridization” refers to the process by which one strand of nucleic acid forms a duplex with, i.e., base pairs with, a complementary strand. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions can be used to identify sequences having strict identity or near-strict identity with the hybridization probe; while intermediate or low stringency hybridization can be used to identify or detect polynucleotide sequence homologs.

Moderate and high stringency hybridization conditions are well known in the art. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tm), where one half of the hybridized nucleic acids are unpaired with the complementary strand. Mismatched nucleic acids within the duplex lower the Tm. Very stringent hybridization conditions involve 68° C. and 0.1×SSC. A nucleic acid encoding a variant metalloprotease can have a Tm reduced by 1° C.-3° C. or more compared to a duplex formed between the nucleic acid of SEQ ID NO: 4 and its identical complement.

Another example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. Those of skill in the art know how to adjust the temperature, ionic strength, etc. to accommodate factors such as probe length and the like.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, the invention provides methods of enriching compositions for one or more molecules of the invention, such as one or more polypeptides or polynucleotides of the invention. A composition is enriched for a molecule when there is a substantial increase in the concentration of the molecule after application of a purification or enrichment technique. A substantially pure polypeptide or polynucleotide of the invention (e.g., substantially pure metalloprotease polypeptide or polynucleotide encoding a metalloprotease polypeptide of the invention, respectively) will typically comprise at least about 55%, about 60%, about 65%, about 70%, about 75%, about 80%, about 85%, about 90%, about 91%, about 92%, about 93%, about 94%, about 95%, about 96%, about 97%, about 98, about 99%, about 99.5% or more by weight (on a molar basis) of all macromolecular species in a particular composition.

The term “enriched” refers to a compound, polypeptide, cell, nucleic acid, amino acid, or other specified material or component that is present in a composition at a relative or absolute concentration that is higher than a starting composition.

In a related sense, the invention provides methods of enriching compositions for one or more molecules of the invention, such as one or more polypeptides of the invention (e.g., one or more metalloprotease polypeptides of the invention) or one or more nucleic acids of the invention (e.g., one or more nucleic acids encoding one or more metalloprotease polypeptides of the invention). A composition is enriched for a molecule when there is a substantial increase in the concentration of the molecule after application of a purification or enrichment technique. A substantially pure polypeptide or polynucleotide will typically comprise at least about 55%, about 60%, about 65%, about 70%, about 75%, about 80%, about 85%, about 90%, about 91%, about 92%, about 93%, about 94%, about 95%, about 96%, about 97%, about 98, about 99%, about 99.5% or more by weight (on a molar basis) of all macromolecular species in a particular composition.

As used herein, the term “functional assay” refers to an assay that provides an indication of a protein's activity. In some embodiments, the term refers to assay systems in which a protein is analyzed for its ability to function in its usual capacity. For example, in the case of a protease, a functional assay involves determining the effectiveness of the protease to hydrolyze a proteinaceous substrate.

The terms “modified nucleic acid sequence” and “modified gene” are used interchangeably herein to refer to a nucleic acid sequence that includes a deletion, insertion or interruption of naturally occurring (i.e., wild-type) nucleic acid sequence. In some embodiments, the expression product of the modified nucleic acid sequence is a truncated protein (e.g., if the modification is a deletion or interruption of the sequence). In some embodiments, the truncated protein retains biological activity. In alternative embodiments, the expression product of the modified nucleic acid sequence is an elongated protein (e.g., modifications comprising an insertion into the nucleic acid sequence). In some embodiments, a nucleotide insertion in the nucleic acid sequence leads to a truncated protein (e.g., when the insertion results in the formation of a stop codon). Thus, an insertion may result in either a truncated protein or an elongated protein as an expression product.

A “mutant” nucleic acid sequence typically refers to a nucleic acid sequence that has an alteration in at least one codon occurring in a host cell's wild-type sequence such that the expression product of the mutant nucleic acid sequence is a protein with an altered amino acid sequence relative to the wild-type protein. The expression product may have an altered functional capacity (e.g., enhanced enzymatic activity).

As used herein, the phrase “alteration in substrate specificity” refers to changes in the substrate specificity of an enzyme. In some embodiments, a change in substrate specificity is defined as a change in k_{cat} and/or Kₘ for a particular substrate, resulting from mutations of the enzyme or alteration of reaction conditions. The substrate specificity of an enzyme is determined by comparing the catalytic efficiencies it exhibits with different substrates. These determinations find particular use in assessing the efficiency of mutant enzymes, as it is generally desired to produce variant enzymes that exhibit greater ratios of k_{cat}/Kₘ for substrates of interest. However, it is not intended that the present invention be limited to any particular substrate composition or substrate specificity.

As used herein, “surface property” is used in reference to electrostatic charge, as well as properties such as the hydrophobicity and hydrophilicity exhibited by the surface of a protein.

As used herein, the term “net charge” is defined as the sum of all charges present in a molecule. “Net charge changes” are made to a parent protein molecule to provide a variant that has a net charge that differs from that of the parent molecule (i.e., the variant has a net charge that is not the same as that of the parent molecule). For example, substitution of a neutral amino acid with a negatively charged amino acid or a positively charged amino acid with a neutral amino acid results in net charge of −1 with respect to the parent molecule. Substitution of a positively charged amino acid with a negatively charged amino acid results in a net charge of −2 with respect to the parent. Substitution of a neutral amino acid with a positively charged amino acid or a negatively charged amino acid with a neutral amino acid results in net charge of +1 with respect to the parent. Substitution of a negatively charged amino acid with a positively charged amino acid results in a net charge of +2 with respect to the parent. The net charge of a parent protein can also be altered by deletion and/or insertion of charged amino acids. A net change change applies to changes in charge of a variant versus a parent when measured at the same pH conditions.

The terms “thermally stable” and “thermostable” and “thermostability” refer to proteases that retain a specified amount of enzymatic activity after exposure to identified temperatures over a given period of time under conditions prevailing during the proteolytic, hydrolyzing, cleaning or other process of the invention, while being exposed to altered temperatures. “Altered temperatures” encompass increased or decreased temperatures. In some embodiments, the proteases retain at least about 50%, about 60%, about 70%, about 75%, about 80%, about 85%, about 90%, about 92%, about 95%, about 96%, about 97%, about 98%, or about 99% proteolytic activity after exposure to altered temperatures over a given time period, for example, at least about 60 minutes, about 120 minutes, about 180 minutes, about 240 minutes, about 300 minutes, etc.

The term “enhanced stability” in the context of an oxidation, chelator, thermal, chemical, autolytic and/or pH stable protease refers to a higher retained proteolytic activity over time as compared to other proteases (e.g., thermolysin proteases) and/or wild-type enzymes.

The term “diminished stability” in the context of an oxidation, chelator, thermal and/or pH stable protease refers to a lower retained proteolytic activity over time as compared to other proteases (e.g., thermolysin proteases) and/or wild-type enzymes.

The term “cleaning activity” refers to a cleaning performance achieved by a metalloprotease polypeptide or reference protease under conditions prevailing during the proteolytic, hydrolyzing, cleaning, or other process of the invention. In some embodiments, cleaning performance of a metalloprotease polypeptide or reference protease may be determined by using various assays for cleaning one or more various enzyme sensitive stains on an item or surface (e.g., a stain resulting from food, grass, blood, ink, milk, oil, and/or egg protein). Cleaning performance of a variant or reference protease can be determined by subjecting the stain on the item or surface to standard wash condition(s) and assessing the degree to which the stain is removed by using various chromatographic, spectrophotometric, or other quantitative methodologies. Exemplary cleaning assays and methods are known in the art and include, but are not limited to those described in WO 99/34011 and U.S. Pat. No. 6,605,458, both of which are herein incorporated by reference, as well as those cleaning assays and methods included in the Examples provided below.

The term “cleaning effective amount” of a metalloprotease polypeptide or reference protease refers to the amount of protease that achieves a desired level of enzymatic activity in a specific cleaning composition. Such effective amounts are readily ascertained by one of ordinary skill in the art and are based on many factors, such as the particular protease used, the cleaning application, the specific composition of the cleaning composition, and whether a liquid or dry (e.g., granular, tablet, bar) composition is required, etc.

The term “enhanced performance” in the context of cleaning activity refers to an increased or greater cleaning activity by an enzyme with respect to a parent or reference protein as measured on certain enzyme sensitive stains such as egg, milk, grass, ink, oil, and/or blood, as determined by usual evaluation after a standard wash cycle and/or multiple wash cycles.

The term “diminished performance” in the context of cleaning activity refers to a decreased or lesser cleaning activity by an enzyme on certain enzyme sensitive stains such as egg, milk, grass or blood, as determined by usual evaluation after a standard wash cycle and/or multiple wash cycles.

Cleaning compositions and cleaning formulations include any composition that is suited for cleaning, bleaching, disinfecting, and/or sterilizing any object, item, and/or surface. Such compositions and formulations include, but are not limited to for example, liquid and/or solid compositions, including cleaning or detergent compositions (e.g., liquid, tablet, gel, bar, granule, unit dose and/or solid laundry cleaning or detergent compositions and fine fabric detergent compositions; hard surface cleaning compositions and formulations, such as for glass, wood, ceramic and metal counter tops and windows; carpet cleaners; oven cleaners; fabric fresheners; fabric softeners; and textile, laundry booster cleaning or detergent compositions, laundry additive cleaning compositions, and laundry pre-spotter cleaning compositions; dishwashing compositions, including hand or manual dishwash compositions (e.g., “hand” or “manual” dishwashing detergents) and automatic dishwashing compositions (e.g., “automatic dishwashing detergents”).

As used herein, the term “bleaching” refers to the treatment of a material (e.g., fabric, laundry, pulp, etc.) or surface for a sufficient length of time and/or under appropriate pH and/or temperature conditions to effect a brightening (i.e., whitening) and/or cleaning of the material. Examples of chemicals suitable for bleaching include, but are not limited to, for example, ClO₂, H₂O₂, peracids, NO₂, etc.

As used herein, “wash performance” of a protease (e.g., a metalloprotease polypeptide of the invention) refers to the contribution of a metalloprotease polypeptide to washing that provides additional cleaning performance to the detergent as compared to the detergent without the addition of the metalloprotease polypeptide to the composition. Wash performance is compared under relevant washing conditions. In some test systems, other relevant factors, such as detergent composition, sud concentration, water hardness, washing mechanics, time, pH, and/or temperature, can be controlled in such a way that condition(s) typical for household application in a certain market segment (e.g., hand or manual dishwashing, automatic dishwashing, dishware cleaning, tableware cleaning, fabric cleaning, etc.) are imitated.

The term “relevant washing conditions” is used herein to indicate the conditions, particularly washing temperature, time, washing mechanics, sud concentration, type of detergent and water hardness, actually used in households in a hand dishwashing, automatic dishwashing, or laundry detergent market segment.

The term “improved wash performance” is used to indicate that a better end result is obtained in stain removal under relevant washing conditions, or that less metalloprotease polypeptide, on weight basis, is needed to obtain the same end result relative to the corresponding wild-type or starting parent protease.

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, the filler salt is sodium sulfate.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

Oligonucleotide synthesis and purification steps are typically performed according to specifications. Techniques and procedures are generally performed according to conventional methods well known in the art and various general references that are provided throughout this document. Procedures therein are believed to be well known to those of ordinary skill in the art and are provided for the convenience of the reader.

### Variant Metalloprotease Polypeptides of the Present Invention

In some embodiments, the present invention provides novel variant metalloprotease enzyme polypeptides having a modification in a calcium binding region. In some embodiments, the variant is a variant of a parent or reference sequence. The parent or reference sequence can be, for example, any M4 metalloprotease, or a Bacillus derived metalloprotease, such as Bacillus thermoproteolyticus, Bacillus cereus, or Bacillus subtilis (for example, the sequences of SEQ ID NOs: 13-15), or a Paenibacillus derived metalloprotease, such as the sequences of SEQ ID NOs: 1-12.

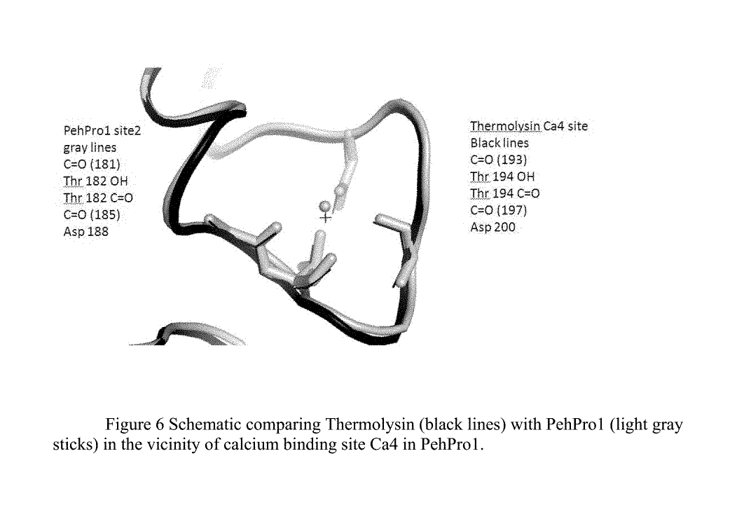

A residue (amino acid) of a metalloprotease is equivalent to a residue of Thermolysin metalloprotease if it is either homologous (i.e., corresponding in position in either primary or tertiary structure) or analogous to a specific residue or portion of that residue in Thermolysin metalloprotease from Bacillus thermoproteolyticus (i.e., having the same or similar functional capacity to react or interact chemically). In order to establish homology to primary structure, the amino acid sequence of a metalloprotease is directly compared to the Thermolysin primary sequence and particularly to a set of residues known to be invariant in diverse M4 metalloproteinases as shown in FIG. 6.1).

After aligning the conserved residues, allowing for necessary insertions and deletions in order to maintain alignment (i.e. avoiding the elimination of conserved residues through arbitrary deletions and insertions) the residues equivalent to particular amino acids in the primary sequence of thermolysin are defined. Suitable methods to produce such modifications include those disclosed herein and an example is shown in Example 6. These conserved residues thus may be used to define the corresponding equivalent amino acid residues of Thermolysin and in other M4 metalloproteinases such as the metalloproteinases from Paenibacillus organisms such as PehPro1. These two particular sequences (Thermolysin (1_KEI) and PehPro1) are aligned in FIG. 6.1 to produce the maximum homology of conserved residues. As can be seen, there are a number of insertions and deletions in the PehPro1 sequence as compared to Thermolysin.

The position of an amino acid residue in a given amino acid sequence is typically numbered herein using the numbering of the position of the corresponding amino acid residue of the Bacillus thermoproteolyticus metalloprotease Thermolysin amino acid sequence shown in SEQ ID NO: 13. The Bacillus thermoproteolyticus metalloprotease Thermolysin amino acid sequence of SEQ ID NO: 13 thus serves as a reference parent sequence. A given amino acid sequence, such as a metalloprotease enzyme amino acid sequence and variants thereof described herein, can be aligned with the Thermolysin sequence (SEQ ID NO: 13) using an alignment algorithm as described herein on the primary and/or tertiary structures, and an amino acid residue in the given amino acid sequence that aligns (preferably optimally aligns) with an amino acid residue in the Thermolysin sequence can be conveniently numbered by reference to the corresponding amino acid residue in the metalloprotease Thermolysin sequence.

The equivalent amino acid of Asp57 in Thermolysin, in PehPro is the particular Serine shown at that aligned position. In FIG. 6.1, the equivalent amino acid at position 57 in PpoPro1 is again Aspartic acid. Thus, these particular residues in PehPro1, and thermolysin may be substituted by a different amino acid to produce a mutant metalloprotease, since they are equivalent in primary structure to Asp 57 in thermolysin. Equivalent amino acids of course are not limited to those for Asp57 but extend to any residue which is equivalent to a residue in Thermolysin, and this is intended as an example of equivalent residues.

Equivalent residues homologous at the level of tertiary structure for a metalloprotease whose tertiary structure has been determined by x-ray crystallography, are defined as those for which the atomic coordinates of 2 or more of the main chain atoms of a particular amino acid residue of the M4 metalloproteinase and Thermolysin (N on N. CA on CA, C on C, and 0 on 0) are within 0.13 nm and preferably 0.1 nm after superposition. Superposition can be accomplished by superimposing the common secondary structure. This can be accomplished using any one of several known algorithms in the art, such as The PyMOL Molecular Graphics System, Version 1.5.0.4 Schrodinger, LLC or Coot [Emsley et al. (2010) Acta Crystallogr D Biol Crystallogr. 66(Pt4): 486]. Superposition is achieved after the best model has been oriented and positioned to give the maximum overlap of atomic coordinates of non-hydrogen protein atoms of the metalloprotease in question to the Thermolysin metalloprotease from Bacillus thermoproteolyticus. The best model is the crystallographic model determined at the highest resolution, and if more than one, the one giving the lowest R factor.

Equivalent residues which are functionally analogous to a specific residue of Thermolysin are defined as those amino acids of the metalloproteases which may adopt a conformation such that they either alter, modify or contribute to protein structure, substrate binding or catalysis in a manner defined and attributed to a specific residue of thermolysin as described herein. Further, they are those residues of the metalloproteinase (for which a tertiary structure has been obtained by x-ray crystallography), which occupy an analogous position to the extent that although the main chain atoms of the given residue may not satisfy the criteria of equivalence on the basis of occupying a homologous position, the atomic coordinates of at least two of the side chain atoms of the residue lie with 0.13 nm of the corresponding side chain atoms of Thermolysin. The three dimensional structures would be aligned as outlined above.

### Calcium Binding Region

The structure of the M4 class metalloprotease thermolysin has been found to have four calcium-binding regions. The structural information from Thermolysin and other metalloproteases can be used to determine modifications that can be made to M4 class metalloproteases in order to remove calcium binding.

Based largely on analysis of the three-dimensional structure of thermolysin, it has been discovered that M4 class metalloproteases can have as many as four calcium binding sites. In thermolysin, there is a double cation binding site, herein referred to as Ca1-2, which has a calcium binding region including residues 136, 138 and 177-190 using the numbering of thermolysin from Bacillus thermoproteolyticus found in SEQ ID NO:13. There is also a calcium binding site, herein referred to as Ca3, which has a calcium binding region including residues 55-66, and a fourth binding site referred to as Ca4, which has a calcium binding region including residues 193-200.

In some embodiments, it is desirable to be able to decrease the Ca²⁺ dependency of a metalloprotease. As such, in some embodiments, the invention is a variant of a parent metalloprotease, such as a M4 class metalloprotease or a variant of any one of SEQ ID NOs: 1-15, which exhibits protease activity and which has a decreased Ca²⁺ dependency as compared to the parent metalloprotease. The decreased Ca²⁺ dependency has the functional result that the variant exhibits proteolytic activity in the presence of a lower concentration of calcium ion in the extraneous medium than is necessary for the parent enzyme and, for example, therefore is less sensitive than the parent to calcium ion-depleting conditions such as those obtained in media containing calcium-complexing agents (such as certain detergent builders). In some embodiments, the variant retains at least 60%, 65%, 70%, 75%, 80%, 85%, 90%, 95%, 96%, 97%, 98%, 99%, 100% or even has greater activity compared to the parent metalloprotease. This can be measured in a proteolytic assay, such as those described in Example 1.

### Calcium Binding

A stabilization strategy based around decreased calcium binding can improve enzyme stability in environments with decreased availability of free calcium ions. One of the major industrial uses of subtilisins is in environments containing high concentrations of metal chelators. Additionally, because these calcium binding regions are found in various M4 metalloproteases, it is expected that equivalent mutations for other M4 metalloproteases will likewise eliminate calcium binding and provide for enzymatically active variants. These calcium binding regions can also be found in various metalloproteases that are not categorized as M4 metalloproteases, but share the same properties as an M4 metalloprotease, including the calcium binding regions.

In some embodiments, the invention is a metalloprotease polypeptide comprising a calcium binding region. In some embodiments, the above polypeptide comprises a modification in at least one amino acid residue in one of the calcium binding regions, Ca1-2, Ca3 and Ca4, (including residues 55-66, 136, 138, 177-190, and 193-200) of the polypeptide, wherein the amino acid positions of the polypeptide are numbered by correspondence with the amino acid sequence of Bacillus thermoproteolyticus metalloprotease set forth in SEQ ID NO: 13.

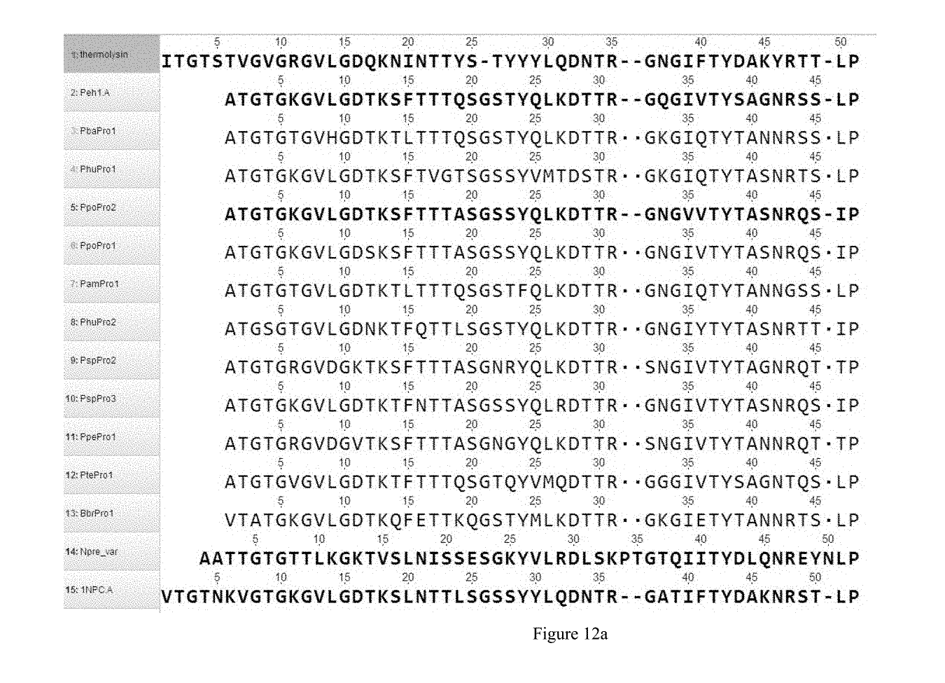

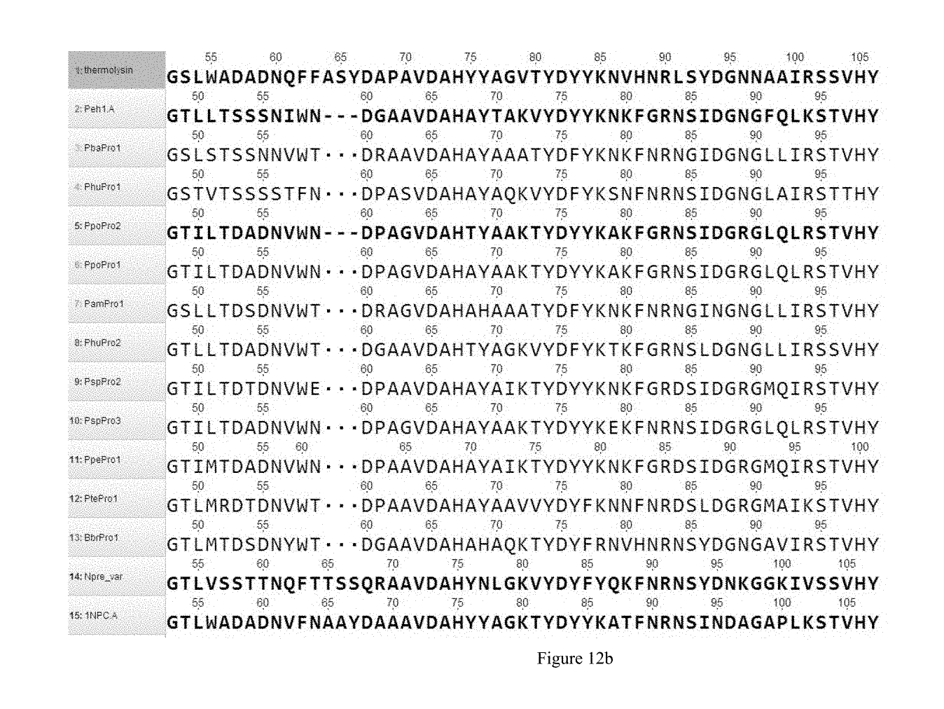

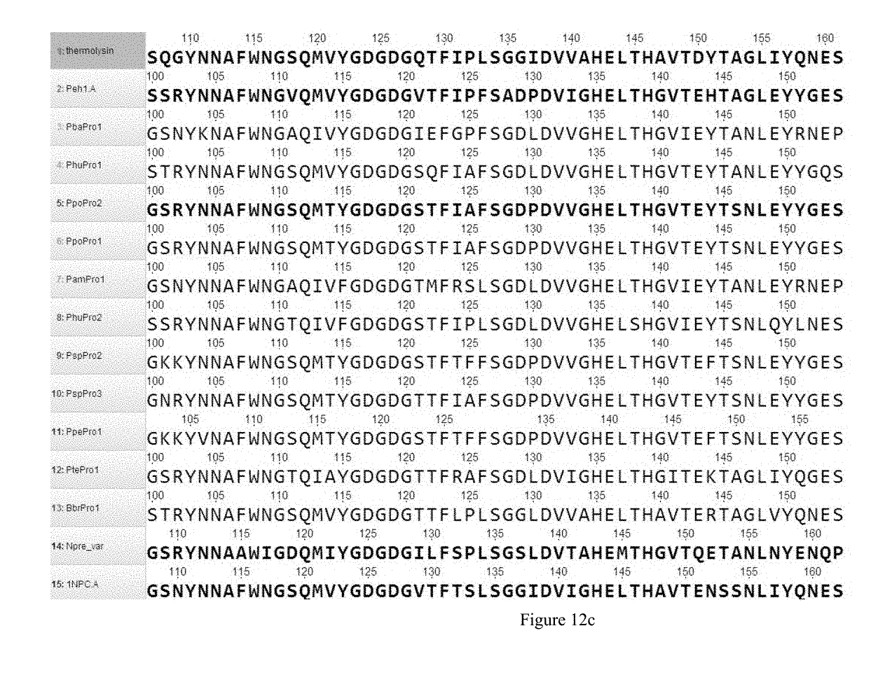

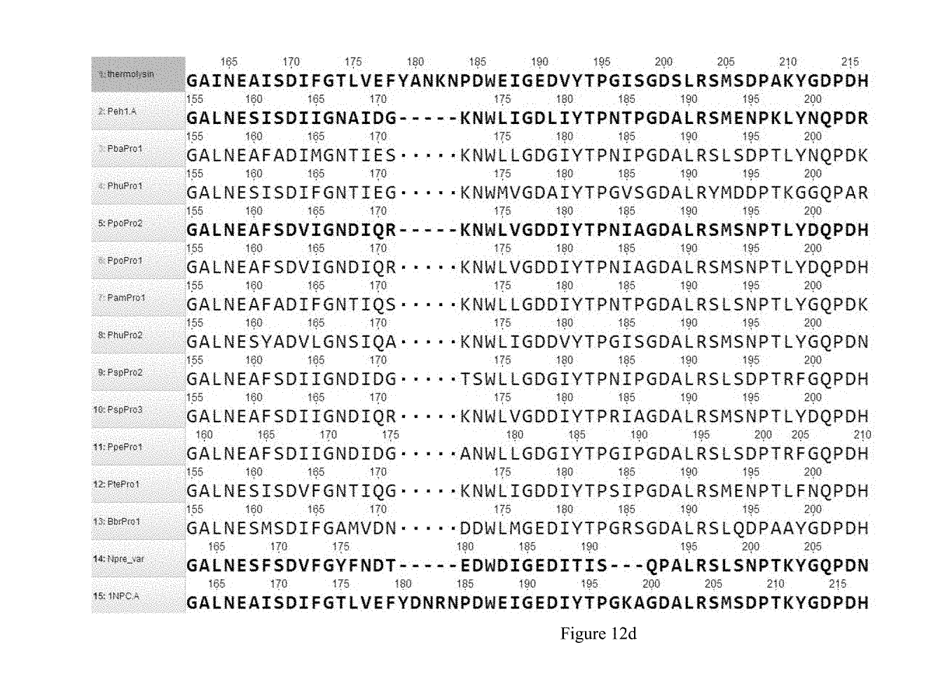

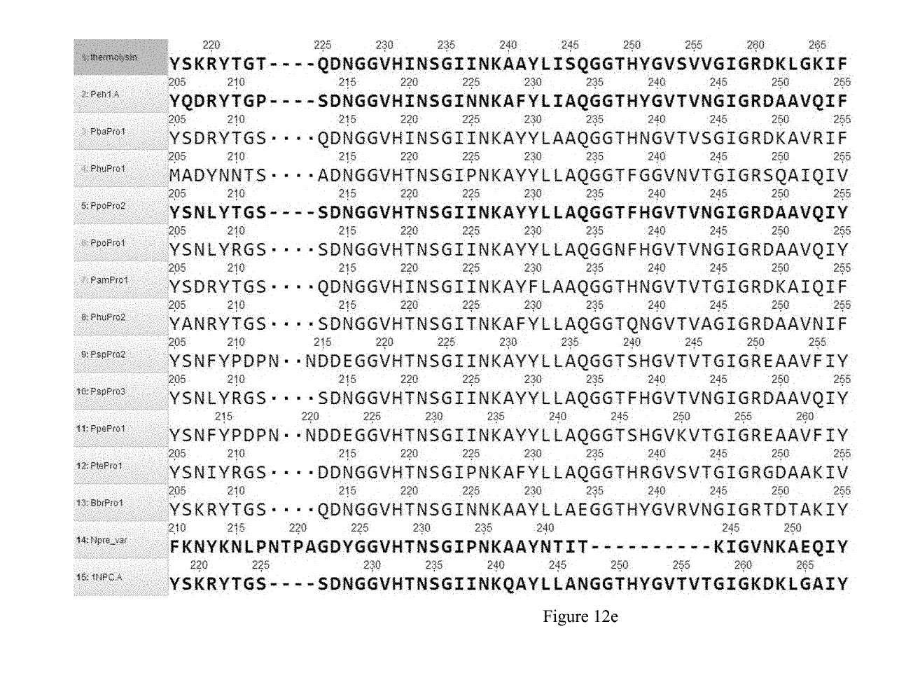

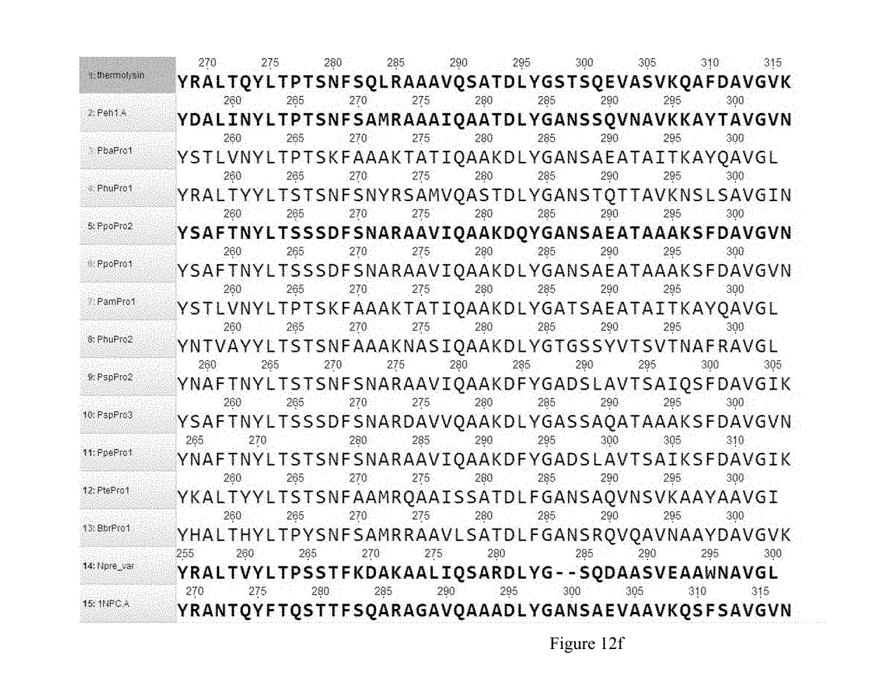

In any of the above embodiments and in new embodiments, the variant comprises a modification in at least one amino acid residue in a calcium binding region Ca1-2 of residues 177-190 of a parent M4 metalloprotease. In any of the above embodiments and in new embodiments, the polypeptide at position 184 is a lysine, threonine, alanine, glutamic acid or aspartic acid. In any of the above embodiments and in new embodiments, the polypeptide at position 185 is a residue other than aspartic acid. In some embodiments, the polypeptide at position 185 is a non-negatively charged residue; in other embodiments, the polypeptide at position 185 is a neutrally charged residue; and in yet other embodiments, the polypeptide at position 185 is an asparagine or serine. In any of the above embodiments and in new embodiments, the polypeptide at position 187 is a non-negatively charged residue. In some embodiments, the polypeptide at position 187 is a neutrally charged residue; and in other embodiments, the polypeptide at position 187 is a leucine or methionine; and in yet other embodiments, the polypeptide at position 187 is aspartic acid. In any of the above embodiments and in new embodiments, the polypeptide at position 188 is a leucine, valine, or methionine. In any of the above embodiments and in new embodiments, the polypeptide at position 190 is a residue other than glutamic acid. In some embodiments, the polypeptide at position 190 is aspartic acid. In sequence alignment of Ca1-2 regions, there are conserved residues glycine at position 173 and tryptophan at position 186 (see FIG. 12). In any of the above embodiments and in new embodiments, the polypeptide comprises seven amino acid residues between the glycine residue at position 173 and tryptophan at position 186. In any of the above embodiments and in new embodiments, the polypeptide comprises a deletion of five amino acid residues between the glycine residue at position 173 and tryptophan at position 186. In structural alignment of Ca1-2 regions, there is a known loop structure between residues 176 and 186 (inclusive of amino acids 177 and 185 in the loop). In any of the above embodiments and in new embodiments, the polypeptide comprises a seven amino acid loop sequence between positions 177 to 185 which is replaced by a two amino acid sequence. Without being bound by theory, a polypeptide of the instant invention has improved stability by replacement of the loop structure with a shorter amino acid sequence that can span the region between positions 177 to 185, and in particular, a two amino acid sequence is preferred to span the region between positions 177 to 185. In any of the above embodiments, the two amino acid sequence contains at least one positively charged amino acid, and in some embodiments, the positively charged amino acid is lysine. In any of the above embodiments and in new embodiments, the polypeptide comprises a deletion at amino acid residue positions 179-183. In any of the above embodiments and in new embodiments, the polypeptide at position 177 is a neutrally charged residue or aspartic acid; and in some embodiments, the neutrally charged residue is glutamine. In any of the above embodiments and in new embodiments, the polypeptide at position 178 is a glycine, serine, arginine, alanine, asparagines, and threonine. In any of the above embodiments and in new embodiments, the polypeptide at position 136 is aspartic acid or serine.

In any of the above embodiments and in new embodiments, the invention is a metalloprotease polypeptide having a modification in at least one amino acid residue in a calcium binding region Ca3 of residues 55-66, wherein the amino acid positions of the variant are numbered by correspondence with the amino acid sequence of Bacillus proteolyticus metalloprotease set forth in SEQ ID NO: 13. In any of the above embodiments and in new embodiments, the polypeptide at position 55 is a leucine, serine, valine, and methionine. In any of the above embodiments and in new embodiments, the polypeptide at position 56 is a serine, arginine and threonine. In any of the above embodiments and in new embodiments, the polypeptide at position 57 is a serine. In any of the above embodiments and in new embodiments, the polypeptide at position 58 is a serine and threonine. In any of the above embodiments and in new embodiments, the polypeptide at position 59 is a serine, threonine, and asparagine. In any of the above embodiments and in new embodiments, the polypeptide has a serine at position 57, serine at position 58 and serine or asparagines at position 59. In any of the above embodiments and in new embodiments, the polypeptide at position 60 is a serine. In any of the above embodiments and in new embodiments, the polypeptide at position 61 is an isoleucine, valine, and threonine. In any of the above embodiments and in new embodiments, the polypeptide at position 62 is a tryptophan and phenylalanine. In any of the above embodiments and in new embodiments, the polypeptide at position 63 is a asparagine, glutamic acid, and threonine. In sequence alignment of the Ca3 region, there are conserved residues phenylalanine/tryptophan at position 62 and aspartic acid at position 67 (see FIG. 12). In any of the above embodiments and in new embodiments, the polypeptide comprises four amino acid residues between the phenylalanine/tryptophan residue at position 62 and aspartic acid at position 67. In any of the above embodiments and in new embodiments, the polypeptide comprises a deletion of three amino acid residues between the phenylalanine/tryptophan residue at position 62 and aspartic acid at position 67. In structural alignment of the Ca3 region, there is a known loop structure between residues 62 and 67 (inclusive of amino acids 62 and 67 in the loop). In any of the above embodiments and in new embodiments, the polypeptide comprises a four amino acid loop sequence between positions 62 to 67 which is replaced by a one amino acid sequence. Without being bound by theory, a polypeptide of the instant invention has improved stability by replacement of the loop structure with a shorter amino acid sequence that can span the region between positions 62 to 67, and in particular, a one amino acid sequence is preferred to span the region between positions 62 to 67. In any of the above embodiments, the one amino acid sequence is an asparagines, threonine, or glutamic acid. In any of the above embodiments and in new embodiments, the polypeptide comprises a deletion at amino acid residue positions 64-66.

In any of the above embodiments and in new embodiments, the invention is a metalloprotease polypeptide having a modification in at least one amino acid residue in a calcium binding region Ca4 of residues 193-200, wherein the amino acid positions of the variant are numbered by correspondence with the amino acid sequence of Bacillus proteolyticus metalloprotease set forth in SEQ ID NO: 13. In any of the above embodiments and in new embodiments, the polypeptide at position 193 is a threonine. In any of the above embodiments and in new embodiments, the polypeptide at position 194 is a isoleucine. In any of the above embodiments and in new embodiments, the polypeptide at position 195 is a serine. In any of the above embodiments and in new embodiments, the polypeptide comprises a deletion at amino acid residue positions 196-198. In any of the above embodiments and in new embodiments, the polypeptide at position 199 is a glutamine. In any of the above embodiments and in new embodiments, the polypeptide at position 200 is a proline.

In some embodiments of the invention, the calcium binding region Ca1-2 has been modified to bind fewer than two calcium ions. In some embodiments of the invention, the calcium binding region Ca3 has been modified to bind fewer than one calcium ion. In some embodiments of the invention, the calcium binding region Ca4 has been modified to bind fewer than one calcium ion.

In some embodiments, the invention is a variant metalloprotease of a parent metalloprotease polypeptide. In some embodiments, the variant comprises a modification in a calcium binding region of the parent polypeptide. In some embodiments, the variant comprises a modification to any of the amino acids listed above. In some embodiments, the parent polypeptide is an M4 metalloprotease. In some embodiments, the metalloprotease polypeptide of the present invention has at least 60, 65, 70, 75, 80, 85, 90, 95, 96, 97, 98, 99, or 100% sequence identity to the parent polypeptide. In some embodiments, the metalloprotease polypeptide of the present invention has at least 60, 65, 70, 75, 80, 85, 90, 95, 96, 97, 98, 99, or 100% sequence identity to any of the sequences found in SEQ ID NOs: 1-15. In some embodiments, the metalloprotease polypeptide of the present invention has at least 60, 65, 70, 75, 80, 85, 90, 95, 96, 97, 98, 99, or 100% sequence identity to SEQ ID NO:13.

In some embodiments, the invention is a variant metalloprotease having immunological cross-reactivity with any of the variant metalloproteases described above. Immunological cross-reactivity can be assayed using an antibody raised against or reactive with at least one epitope of any of the variant metalloproteases listed above. The antibody, which can either be monoclonal or polyclonal, can be produced by methods known in the art. Immunological cross-reactivity can be measured using assays known in the art, such as Western blotting, radial immunodiffusion assay, or enzyme-linked immunosorbant assay (ELISA).

### Metalloprotease Polypeptides of the Present Invention

The present invention provides novel metalloprotease enzyme polypeptides, which may be collectively referred to as “enzymes of the invention” or “polypeptides of the invention.” Polypeptides of the invention include isolated, recombinant, substantially pure, or non-naturally occurring polypeptides. In some embodiments, the invention includes variants, as described above, of M4 class metalloproteases. In some embodiments, polypeptides of the invention are useful in cleaning applications and can be incorporated into cleaning compositions that are useful in methods of cleaning an item or a surface in need of cleaning.

In some embodiments, the enzyme of the present invention has 50, 60, 65, 70, 75, 80, 85, 90, 91, 92, 93, 94, 95, 96, 97, 98, 99 or 100% identity to a M4 class metalloprotease. In some embodiments, the enzyme of the present invention has 50, 60, 65, 70, 75, 80, 85, 90, 91, 92, 93, 94, 95, 96, 97, 98, 99 or 100% identity to SEQ ID NO: 13. In various embodiments, the enzyme of the present invention has 50, 60, 65, 70, 75, 80, 85, 90, 91, 92, 93, 94, 95, 96, 97, 98, 99 or 100% identity to a metalloprotease enzyme from any of SEQ ID NO:1-15.

In some embodiments, the invention includes an isolated, recombinant, substantially pure, or non-naturally occurring enzyme having protease activity, which polypeptide comprises a polypeptide sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to a parent enzyme as provided herein.

In some embodiments, the polypeptide of the present invention, is a polypeptide having a specified degree of amino acid sequence homology to the exemplified polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to the amino acid sequence of any of SEQ ID NO: 1-15. Homology can be determined by amino acid sequence alignment, e.g., using a program such as BLAST, ALIGN, or CLUSTAL, as described herein.

Also provided is a polypeptide enzyme of the present invention, having protease activity, said enzyme comprising an amino acid sequence which differs from the amino acid sequence of any of SEQ ID NO:1-15 by no more than 50, no more than 40, no more than 30, no more than 35, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

As noted above, the variant enzyme polypeptides of the invention have enzymatic activities (e.g., protease activities) and thus are useful in cleaning applications, including but not limited to, methods for cleaning dishware items, tableware items, fabrics, and items having hard surfaces (e.g., the hard surface of a table, table top, wall, furniture item, floor, ceiling, etc.). Exemplary cleaning compositions comprising one or more variant metalloprotease enzyme polypeptides of the invention are described infra. The enzymatic activity (e.g., protease enzyme activity) of an enzyme polypeptide of the invention can be determined readily using procedures well known to those of ordinary skill in the art. The Examples presented infra describe methods for evaluating the enzymatic activity and cleaning performance. The performance of polypeptide enzymes of the invention in removing stains (e.g., a protein stain such as blood/milk/ink or egg yolk), cleaning hard surfaces, or cleaning laundry, dishware or tableware item(s), or cleaning contact lenses can be readily determined using procedures well known in the art and/or by using procedures set forth in the Examples.

The metalloprotease polypeptides of the present invention can have protease activity over a broad range of pH conditions. In some embodiments, the metalloprotease polypeptides have protease activity on azo-casein as a substrate, as demonstrated in Example 3. In some embodiments, the metalloprotease polypeptides have protease activity at a pH of from about 3.0 to about 12.0. In some embodiments, the metalloprotease polypeptides have protease activity at a pH of from about 4.0 to about 11.0.

In some embodiments, the metalloprotease polypeptides of the present invention have protease activity at a temperature range of from about 10° C. to about 100° C. In some embodiments, the metalloprotease polypeptides of the present invention have protease activity at a temperature range of from about 20° C. to about 90° C.

In some embodiments, the metalloprotease polypeptides of the present invention demonstrate cleaning performance in a cleaning composition. Cleaning compositions often include ingredients harmful to the stability and performance of enzymes, making cleaning compositions a harsh environment for enzymes, e.g. metalloproteases, to retain function. Thus, it is not trivial for an enzyme to be put in a cleaning composition and expect enzymatic function (e.g. metalloprotease activity, such as demonstrated by cleaning performance). In some embodiments, the metalloprotease polypeptides of the present invention demonstrate cleaning performance in automatic dishwashing (ADW) detergent compositions. In some embodiments, the cleaning performance in automatic dishwashing (ADW) detergent compositions includes cleaning of egg yolk stains. In some embodiments, the metalloprotease polypeptides of the present invention demonstrate cleaning performance in laundry detergent compositions. In some embodiments, the cleaning performance in laundry detergent compositions includes cleaning of blood/milk/ink stains. In each of the cleaning compositions, the metalloprotease polypeptides of the present invention demonstrate cleaning performance with or without a bleach component.

A polypeptide of the invention can be subject to various changes, such as one or more amino acid insertions, deletions, and/or substitutions, either conservative or non-conservative, including where such changes do not substantially alter the enzymatic activity of the polypeptide. Similarly, a nucleic acid of the invention can also be subject to various changes, such as one or more substitutions of one or more nucleotides in one or more codons such that a particular codon encodes the same or a different amino acid, resulting in either a silent variation (e.g., when the encoded amino acid is not altered by the nucleotide mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence of the invention can also be modified to include one or more codons that provide for optimum expression in an expression system (e.g., bacterial expression system), while, if desired, said one or more codons still encode the same amino acid(s).

In some embodiments, the present invention provides a genus of enzyme polypeptides having the desired enzymatic activity (e.g., protease enzyme activity or cleaning performance activity) which comprise sequences having the amino acid substitutions described herein and also which comprise one or more additional amino acid substitutions, such as conservative and non-conservative substitutions, wherein the polypeptide exhibits, maintains, or approximately maintains the desired enzymatic activity (e.g., proteolytic activity, as reflected in the cleaning activity or performance of the polypeptide enzyme of SEQ ID NO: 13). Amino acid substitutions in accordance with the invention may include, but are not limited to, one or more non-conservative substitutions and/or one or more conservative amino acid substitutions. A conservative amino acid residue substitution typically involves exchanging a member within one functional class of amino acid residues for a residue that belongs to the same functional class (conservative amino acid residues are considered functionally homologous or conserved in calculating percent functional homology). A conservative amino acid substitution typically involves the substitution of an amino acid in an amino acid sequence with a functionally similar amino acid. For example, alanine, glycine, serine, and threonine are functionally similar and thus may serve as conservative amino acid substitutions for one another. Aspartic acid and glutamic acid may serve as conservative substitutions for one another. Asparagine and glutamine may serve as conservative substitutions for one another. Arginine, lysine, and histidine may serve as conservative substitutions for one another. Isoleucine, leucine, methionine, and valine may serve as conservative substitutions for one another. Phenylalanine, tyrosine, and tryptophan may serve as conservative substitutions for one another.

Other conservative amino acid substitution groups can be envisioned. For example, amino acids can be grouped by similar function or chemical structure or composition (e.g., acidic, basic, aliphatic, aromatic, sulfur-containing). For instance, an aliphatic grouping may comprise: Glycine (G), Alanine (A), Valine (V), Leucine (L), Isoleucine (I). Other groups containing amino acids that are considered conservative substitutions for one another include: aromatic: Phenylalanine (F), Tyrosine (Y), Tryptophan (W); sulfur-containing: Methionine (M), Cysteine (C); Basic: Arginine (R), Lysine (K), Histidine (H); Acidic: Aspartic acid (D), Glutamic acid (E); non-polar uncharged residues, Cysteine (C), Methionine (M), and Proline (P); hydrophilic uncharged residues: Serine (S), Threonine (T), Asparagine (N), and Glutamine (Q). Additional groupings of amino acids are well-known to those of skill in the art and described in various standard textbooks. Listing of a polypeptide sequence herein, in conjunction with the above substitution groups, provides an express listing of all conservatively substituted polypeptide sequences.

More conservative substitutions exist within the amino acid residue classes described above, which also or alternatively can be suitable. Conservation groups for substitutions that are more conservative include: valine-leucine-isoleucine, phenylalanine-tyrosine, lysine-arginine, alanine-valine, and asparagine-glutamine.

Conservatively substituted variations of a polypeptide sequence of the invention (e.g., variant metalloproteases of the invention) include substitutions of a small percentage, sometimes less than 25%, 20%, 15%, 14%, 13%, 12%, 11%, 10%, 9%, 8%, 7%, or 6% of the amino acids of the polypeptide sequence, or less than 5%, 4%, 3%, 2%, or 1%, or less than 10, 9, 8, 7, 6, 5, 4, 3, 2, or 1 amino acid substitution of the amino acids of the polypeptide sequence, with a conservatively selected amino acid of the same conservative substitution group.

As described elsewhere herein in greater detail and in the Examples provided herein, polypeptides of the invention may have cleaning abilities that may be compared to known proteases, including known metalloproteases.

### Nucleic Acids of the Invention

The invention provides isolated, non-naturally occurring, or recombinant nucleic acids which may be collectively referred to as “nucleic acids of the invention” or “polynucleotides of the invention”, which encode polypeptides of the invention. Nucleic acids of the invention, including all described below, are useful in recombinant production (e.g., expression) of polypeptides of the invention, typically through expression of a plasmid expression vector comprising a sequence encoding the polypeptide of interest or fragment thereof. As discussed above, polypeptides include metalloprotease polypeptides having enzymatic activity (e.g., proteolytic activity) which are useful in cleaning applications and cleaning compositions for cleaning an item or a surface (e.g., surface of an item) in need of cleaning.

In some embodiments, the invention provides an isolated, recombinant, substantially pure, or non-naturally occurring nucleic acid comprising a nucleotide sequence encoding any polypeptide (including any fusion protein, etc.) of the invention described above in the section entitled “Polypeptides of the Invention” and elsewhere herein. The invention also provides an isolated, recombinant, substantially pure, or non-naturally-occurring nucleic acid comprising a nucleotide sequence encoding a combination of two or more of any polypeptides of the invention described above and elsewhere herein.

The present invention provides nucleic acids encoding a metalloprotease polypeptide of the present invention, wherein the metalloprotease polypeptide is a mature form having proteolytic activity, wherein the amino acid positions of the thermolysin variant are numbered by correspondence with the amino acid sequence of Bacillus thermoproteolyticus metalloprotease polypeptide set forth as SEQ ID NO: 13.

Nucleic acids of the invention can be generated by using any suitable synthesis, manipulation, and/or isolation techniques, or combinations thereof. For example, a polynucleotide of the invention may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the nucleic acids of the invention can be also facilitated by any suitable method known in the art, including but not limited to chemical synthesis using the classical phosphoramidite method (See e.g., Beaucage et al. Tetrahedron Letters 22:1859-69 [1981]); or the method described by Matthes et al. (See, Matthes et al., EMBO J. 3:801-805 [1984], as is typically practiced in automated synthetic methods. Nucleic acids of the invention also can be produced by using an automatic DNA synthesizer. Customized nucleic acids can be ordered from a variety of commercial sources (e.g., The Midland Certified Reagent Company, the Great American Gene Company, Operon Technologies Inc., and DNA2.0). Other techniques for synthesizing nucleic acids and related principles are known in the art (See e.g., Itakura et al., Ann. Rev. Biochem. 53:323 [1984]; and Itakura et al., Science 198:1056 [1984]).

As indicated above, recombinant DNA techniques useful in modification of nucleic acids are well known in the art. For example, techniques such as restriction endonuclease digestion, ligation, reverse transcription and cDNA production, and polymerase chain reaction (e.g., PCR) are known and readily employed by those of skill in the art. Nucleotides of the invention may also be obtained by screening cDNA libraries using one or more oligonucleotide probes that can hybridize to or PCR-amplify polynucleotides which encode a metalloprotease polypeptide polypeptide(s) of the invention. Procedures for screening and isolating cDNA clones and PCR amplification procedures are well known to those of skill in the art and described in standard references known to those skilled in the art. Some nucleic acids of the invention can be obtained by altering a naturally occurring polynucleotide backbone (e.g., that encodes an enzyme or parent protease) by, for example, a known mutagenesis procedure (e.g., site-directed mutagenesis, site saturation mutagenesis, and in vitro recombination).

### Methods for Making Modified Metalloprotease Polypeptides of the Invention

A variety of methods are known in the art that are suitable for generating modified polynucleotides of the invention that encode metalloprotease polypeptides of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., metalloprotease polypeptides) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

### Vectors, Cells, and Methods for Producing Metalloprotease Polypeptides of the Invention

The present invention provides vectors comprising at least one metalloprotease polynucleotide of the invention described herein (e.g., a polynucleotide encoding a metalloprotease polypeptide of the invention described herein), expression vectors or expression cassettes comprising at least one nucleic acid or polynucleotide of the invention, isolated, substantially pure, or recombinant DNA constructs comprising at least one nucleic acid or polynucleotide of the invention, isolated or recombinant cells comprising at least one polynucleotide of the invention, and compositions comprising one or more such vectors, nucleic acids, expression vectors, expression cassettes, DNA constructs, cells, cell cultures, or any combination or mixtures thereof.

In some embodiments, the invention provides recombinant cells comprising at least one vector (e.g., expression vector or DNA construct) of the invention which comprises at least one nucleic acid or polynucleotide of the invention. Some such recombinant cells are transformed or transfected with such at least one vector. Such cells are typically referred to as host cells. Some such cells comprise bacterial cells, including, but are not limited to Bacillus sp. cells, such as B. subtilis cells. The invention also provides recombinant cells (e.g., recombinant host cells) comprising at least one metalloprotease polypeptide of the invention.

In some embodiments, the invention provides a vector comprising a nucleic acid or polynucleotide of the invention. In some embodiments, the vector is an expression vector or expression cassette in which a polynucleotide sequence of the invention which encodes a metalloprotease polypeptide of the invention is operably linked to one or additional nucleic acid segments required for efficient gene expression (e.g., a promoter operably linked to the polynucleotide of the invention which encodes a metalloprotease polypeptide of the invention). A vector may include a transcription terminator and/or a selection gene, such as an antibiotic resistance gene, that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular Biological Methods for Bacillus, John Wiley & Sons [1990]; suitable replicating plasmids for B. subtilis include those listed on p. 92) See also, Perego, Integrational Vectors for Genetic Manipulations in Bacillus subtilis, in Sonenshein et al., [eds.] Bacillus subtilis and Other Gram-Positive Bacteria: Biochemistry, Physiology and Molecular Genetics, American Society for Microbiology, Washington, D.C. [1993], pp. 615-624), and p2JM103BBI.

For expression and production of a protein of interest (e.g., metalloprotease polypeptide) in a cell, at least one expression vector comprising at least one copy of a polynucleotide encoding the metalloprotease polypeptide, and in some instances comprising multiple copies, is transformed into the cell under conditions suitable for expression of the metalloprotease. In some embodiments of the present invention, a polynucleotide sequence encoding the metalloprotease polypeptide (as well as other sequences included in the vector) is integrated into the genome of the host cell, while in other embodiments, a plasmid vector comprising a polynucleotide sequence encoding the metalloprotease polypeptide remains as autonomous extra-chromosomal element within the cell. The invention provides both extrachromosomal nucleic acid elements as well as incoming nucleotide sequences that are integrated into the host cell genome. The vectors described herein are useful for production of the metalloprotease polypeptides of the invention. In some embodiments, a polynucleotide construct encoding the metalloprotease polypeptide is present on an integrating vector that enables the integration and optionally the amplification of the polynucleotide encoding the metalloprotease polypeptide into the host chromosome. Examples of sites for integration are well known to those skilled in the art. In some embodiments, transcription of a polynucleotide encoding a metalloprotease polypeptide of the invention is effectuated by a promoter that is the wild-type promoter for the selected precursor protease. In some other embodiments, the promoter is heterologous to the precursor protease, but is functional in the host cell. Specifically, examples of suitable promoters for use in bacterial host cells include, but are not limited to, for example, the amyE, amyQ, amyL, pstS, sacB, pSPAC, pAprE, pVeg, pHpaII promoters, the promoter of the B. stearothermophilus maltogenic amylase gene, the B. amyloliquefaciens (BAN) amylase gene, the B. subtilis alkaline protease gene, the B. clausii alkaline protease gene the B. pumilis xylosidase gene, the B. thuringiensis cryIIIA, and the B. licheniformis alpha-amylase gene. Additional promoters include, but are not limited to the A4 promoter, as well as phage Lambda PR or PL promoters, and the E. coli lac, trp or tac promoters.

Metalloprotease polypeptides of the present invention can be produced in host cells of any suitable microorganism, including bacteria and fungi. In some embodiments, metalloprotease polypeptides of the present invention can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, the metalloprotease polypeptides are produced by Bacillus sp. host cells. Examples of Bacillus sp. host cells that find use in the production of the metalloprotease polypeptides of the invention include, but are not limited to B. licheniformis, B. lentus, B. subtilis, B. amyloliquefaciens, B. lentus, B. brevis, B. stearothermophilus, B. alkalophilus, B. coagulans, B. circulans, B. pumilis, B. thuringiensis, B. clausii, and B. megaterium, as well as other organisms within the genus Bacillus. In some embodiments, B. subtilis host cells are used for production of metalloprotease polypeptides. U.S. Pat. Nos. 5,264,366 and 4,760,025 (RE 34,606) describe various Bacillus host strains that can be used for producing metalloprotease polypeptide of the invention, although other suitable strains can be used.

Several bacterial strains that can be used to produce metalloprotease polypeptides of the invention include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant Bacillus subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics 73:215-228 [1973]; See also, U.S. Pat. Nos. 4,450,235 and 4,302,544, and EP 0134048, each of which is incorporated by reference in its entirety). The use of B. subtilis as an expression host cells is well known in the art (See e.g., Palva et al., Gene 19:81-87 [1982]; Fahnestock and Fischer, J. Bacteriol., 165:796-804 [1986]; and Wang et al., Gene 69:39-47 [1988]).

In some embodiments, the Bacillus host cell is a Bacillus sp. that includes a mutation or deletion in at least one of the following genes, degU, degS, degR and degQ. In some embodiments, the mutation is in a degU gene, and in some embodiments the mutation is degU(Hy)32 (See e.g., Msadek et al., J. Bacteriol. 172:824-834 [1990]; and Olmos et al., Mol. Gen. Genet. 253:562-567 [1997]). In some embodiments, the Bacillus host comprises a mutation or deletion in scoC4 (See e.g., Caldwell et al., J. Bacteriol. 183:7329-7340 [2001]); spoIIE (See e.g., Arigoni et al., Mol. Microbiol. 31:1407-1415 [1999]); and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain of the invention. In some embodiments, these mutations occur alone, while in other embodiments, combinations of mutations are present. In some embodiments, an altered Bacillus host cell strain that can be used to produce a metalloprotease polypeptide of the invention is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments, the Bacillus sp. host cell comprises a deletion of 9 protease genes (See e.g., U.S. Pat. Appln. Pub. No. 2005/0202535, incorporated herein by reference).

Host cells are transformed with at least one nucleic acid encoding at least one metalloprotease polypeptide of the invention using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Those of skill in the art are well aware of suitable methods for introducing nucleic acid sequences of the invention into Bacillus cells (See e.g., Ferrari et al., “Genetics,” in Harwood et al. [eds.], Bacillus, Plenum Publishing Corp. [1989], pp. 57-72; Saunders et al., J. Bacteriol. 157:718-726 [1984]; Hoch et al., J. Bacteriol. 93:1925-1937 [1967]; Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1986]; Fisher et al., Arch. Microbiol. 139:213-217 [1981]; and McDonald, J. Gen. Microbiol. 130:203 [1984]). Indeed, such methods as transformation, including protoplast transformation and transfection, transduction, and protoplast fusion are well known and suited for use in the present invention. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]; and Weinrauch et al., J. Bacteriol. 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding a metalloprotease polypeptide of the invention (i.e., an intermediate cell is not used to amplify, or otherwise process, the DNA construct or vector prior to introduction into the host cell). Introduction of the DNA construct or vector of the invention into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Palmeros et al., Gene 247:255-264 [2000]).

In some embodiments, the transformed cells of the present invention are cultured in conventional nutrient media. The suitable specific culture conditions, such as temperature, pH and the like are known to those skilled in the art and are well described in the scientific literature. In some embodiments, the invention provides a culture (e.g., cell culture) comprising at least one metalloprotease polypeptide or at least one nucleic acid of the invention.

In some embodiments, host cells transformed with at least one polynucleotide sequence encoding at least one metalloprotease polypeptide of the invention are cultured in a suitable nutrient medium under conditions permitting the expression of the present protease, after which the resulting protease is recovered from the culture. In some embodiments, the protease produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.).

In some embodiments, a metalloprotease polypeptide produced by a recombinant host cell is secreted into the culture medium. A nucleic acid sequence that encodes a purification facilitating domain may be used to facilitate purification of proteins. A vector or DNA construct comprising a polynucleotide sequence encoding a metalloprotease polypeptide may further comprise a nucleic acid sequence encoding a purification facilitating domain to facilitate purification of the metalloprotease polypeptide (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, Calif.) between the purification domain and the heterologous protein also find use to facilitate purification.

Assays for detecting and measuring the enzymatic activity of an enzyme, such as a metalloprotease polypeptide of the invention, are well known. Various assays for detecting and measuring activity of proteases (e.g., metalloprotease polypeptides of the invention), are also known to those of ordinary skill in the art. In particular, assays are available for measuring protease activity that are based on the release of acid-soluble peptides from casein or hemoglobin, measured as absorbance at 280 nm or colorimetrically using the Folin method. Other exemplary assays involve the solubilization of chromogenic substrates (See e.g., Ward, “Proteinases,” in Fogarty (ed.), Microbial Enzymes and Biotechnology, Applied Science, London, [1983], pp. 251-317). Other exemplary assays include, but are not limited to hydrolysis of protein substrates such as casein (azo-casein, dimethyl-casein and other forms), and peptidyl substrates such as succinyl-Ala-Ala-Pro-Phe-para nitroanilide assay (suc-AAPF-pNA)(SEQ ID NO: 24) and the 2,4,6-trinitrobenzene sulfonate sodium salt (TNBS). Numerous additional references known to those in the art provide suitable methods (See e.g., Wells et al., Nucleic Acids Res. 11:7911-7925 [1983]; Christianson et al., Anal. Biochem. 223:119-129 [1994]; and Hsia et al., Anal Biochem. 242:221-227 [1999]).

A variety of methods can be used to determine the level of production of a mature protease (e.g., mature metalloprotease polypeptides of the present invention) in a host cell. Such methods include, but are not limited to, for example, methods that utilize either polyclonal or monoclonal antibodies specific for the protease. Exemplary methods include, but are not limited to enzyme-linked immunosorbent assays (ELISA), radioimmunoassays (MA), fluorescent immunoassays (FIA), and fluorescent activated cell sorting (FACS). These and other assays are well known in the art (See e.g., Maddox et al., J. Exp. Med. 158:1211 [1983]).

In some other embodiments, the invention provides methods for making or producing a mature metalloprotease polypeptide of the invention. A mature metalloprotease polypeptide does not include a signal peptide or a propeptide sequence. Some methods comprise making or producing a metalloprotease polypeptide of the invention in a recombinant bacterial host cell, such as for example, a Bacillus sp. cell (e.g., a B. subtilis cell). In some embodiments, the invention provides a method of producing a metalloprotease polypeptide of the invention, the method comprising cultivating a recombinant host cell comprising a recombinant expression vector comprising a nucleic acid encoding a metalloprotease polypeptide of the invention under conditions conducive to the production of the metalloprotease polypeptide. Some such methods further comprise recovering the metalloprotease polypeptide from the culture.

In some embodiments the invention provides methods of producing a metalloprotease polypeptide of the invention, the methods comprising: (a) introducing a recombinant expression vector comprising a nucleic acid encoding a metalloprotease polypeptide of the invention into a population of cells (e.g., bacterial cells, such as B. subtilis cells); and (b) culturing the cells in a culture medium under conditions conducive to produce the metalloprotease polypeptide encoded by the expression vector. Some such methods further comprise: (c) isolating the metalloprotease polypeptide from the cells or from the culture medium.

### Compositions Having the Metalloprotease Polypeptide of the Present Invention

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. Compositions of the invention include cleaning compositions, such as detergent compositions. In the exemplified detergent compositions, the enzymes levels are expressed by pure enzyme by weight of the total composition and unless otherwise specified, the detergent ingredients are expressed by weight of the total compositions.

As indicated herein, in some embodiments, the cleaning compositions of the present invention further comprise adjunct materials including, but not limited to, surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the metalloprotease polypeptides of the present invention in the cleaning compositions, then suitable methods of keeping the cleaning adjunct materials and the protease(s) separated (i.e., not in contact with each other) until combination of the two components is appropriate are used. Such separation methods include any suitable method known in the art (e.g., gelcaps, encapsulation, tablets, physical separation, etc.).

The cleaning compositions of the present invention are advantageously employed for example, in laundry applications, hard surface cleaning, dishwashing applications, including automatic dishwashing and hand dishwashing, as well as cosmetic applications such as dentures, teeth, hair and skin. In addition, due to the unique advantages of increased effectiveness in lower temperature solutions, the enzymes of the present invention are ideally suited for laundry applications. Furthermore, the enzymes of the present invention find use in granular and liquid compositions.

The metalloprotease polypeptides of the present invention also find use in cleaning additive products. In some embodiments, low temperature solution cleaning applications find use. In some embodiments, the present invention provides cleaning additive products including at least one enzyme of the present invention is ideally suited for inclusion in a wash process when additional bleaching effectiveness is desired. Such instances include, but are not limited to low temperature solution cleaning applications. In some embodiments, the additive product is in its simplest form, one or more proteases. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present invention, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials. Acidic fillers find use to reduce pH. Alternatively, in some embodiments, the cleaning additive includes adjunct ingredients, as more fully described below.

The present cleaning compositions and cleaning additives require an effective amount of at least one of the metalloprotease polypeptides provided herein, alone or in combination with other proteases and/or additional enzymes. The required level of enzyme is achieved by the addition of one or more metalloprotease polypeptides of the present invention. Typically the present cleaning compositions comprise at least about 0.0001 weight percent, from about 0.0001 to about 10, from about 0.001 to about 1, or from about 0.01 to about 0.1 weight percent of at least one of the metalloprotease polypeptides of the present invention.

The cleaning compositions herein are typically formulated such that, during use in aqueous cleaning operations, the wash water will have a pH of from about 4.0 to about 11.5 or even from about 5.0 to about 11.5, or even from about 5.0 to about 8.0, or even from about 7.5 to about 10.5. Liquid product formulations are typically formulated to have a pH from about 3.0 to about 9.0 or even from about 3 to about 5. Granular laundry products are typically formulated to have a pH from about 9 to about 11. Techniques for controlling pH at recommended usage levels include the use of buffers, alkalis, acids, etc., and are well known to those skilled in the art.

Suitable “low pH cleaning compositions” typically have a pH of from about 3 to about 5, and are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine or hydrochloric acid, to provide such cleaning composition with a pH of from about 3 to about 5. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the metalloprotease polypeptide(s) is/are employed in a granular composition or liquid, it is desirable for the metalloprotease polypeptide to be in the form of an encapsulated particle to protect the metalloprotease polypeptide from other components of the granular composition during storage. In addition, encapsulation is also a means of controlling the availability of the metalloprotease polypeptide during the cleaning process. In some embodiments, encapsulation enhances the performance of the metalloprotease polypeptide(s) and/or additional enzymes. In this regard, the metalloprotease polypeptides of the present invention are encapsulated with any suitable encapsulating material known in the art. In some embodiments, the encapsulating material typically encapsulates at least part of the metalloprotease polypeptide(s) of the present invention. Typically, the encapsulating material is water-soluble and/or water-dispersible. In some embodiments, the encapsulating material has a glass transition temperature (Tg) of 0° C. or higher. Glass transition temperature is described in more detail in WO 97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See e.g., EP 0 922 499; U.S. Pat. No. 4,977,252; U.S. Pat. No. 5,354,559, and U.S. Pat. No. 5,935,826). In some embodiments, the encapsulating material is a microsphere made from plastic such as thermoplastics, acrylonitrile, methacrylonitrile, polyacrylonitrile, polymethacrylonitrile and mixtures thereof commercially available microspheres that find use include, but are not limited to those supplied by EXPANCEL® (Stockviksverken, Sweden), and PM 6545, PM 6550, PM 7220, PM 7228, EXTENDOSPHERES®, LUXSIL®, Q-CEL®, and SPHERICEL® (PQ Corp., Valley Forge, Pa.).

As described herein, the metalloprotease polypeptides of the present invention find particular use in the cleaning industry, including, but not limited to laundry and dish detergents. These applications place enzymes under various environmental stresses. The metalloprotease polypeptides of the present invention provide advantages over many currently used enzymes, due to their stability under various conditions.

Indeed, there are a variety of wash conditions including varying detergent formulations, wash water volumes, wash water temperatures, and lengths of wash time, to which proteases involved in washing are exposed. In addition, detergent formulations used in different geographical areas have different concentrations of their relevant components present in the wash water. For example, European detergents typically have about 4500-5000 ppm of detergent components in the wash water, while Japanese detergents typically have approximately 667 ppm of detergent components in the wash water. In North America, particularly the United States, detergents typically have about 975 ppm of detergent components present in the wash water.

A low detergent concentration system includes detergents where less than about 800 ppm of the detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of the detergent components are present in the wash water. North American detergents are generally considered to be medium detergent concentration systems as they have approximately 975 ppm of detergent components present in the wash water. Brazil typically has approximately 1500 ppm of detergent components present in the wash water.

A high detergent concentration system includes detergents where greater than about 2000 ppm of the detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. As mentioned above, Brazil typically has approximately 1500 ppm of detergent components present in the wash water. However, other high suds phosphate builder detergent geographies, not limited to other Latin American countries, may have high detergent concentration systems up to about 6000 ppm of detergent components present in the wash water.

In light of the foregoing, it is evident that concentrations of detergent compositions in typical wash solutions throughout the world varies from less than about 800 ppm of detergent to about 6000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 975 ppm of detergent within the wash solution about 62.79 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 40° C. (e.g., about 20° C.), whereas the temperature of wash water in Europe is typically between about 30 and about 60° C. (e.g., about 40° C.). However, in the interest of saving energy, many consumers are switching to using cold water washing. In addition, in some further regions, cold water is typically used for laundry, as well as dish washing applications. In some embodiments, the “cold water washing” of the present invention utilizes “cold water detergent” suitable for washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Accordingly, in some embodiments, the present invention provides metalloprotease polypeptides that show surprising wash performance in at least one set of wash conditions (e.g., water temperature, water hardness, and/or detergent concentration). In some embodiments, the metalloprotease polypeptides of the present invention are comparable in wash performance to other metalloprotease polypeptide proteases. In some embodiments of the present invention, the metalloprotease polypeptides provided herein exhibit enhanced oxidative stability, enhanced thermal stability, enhanced cleaning capabilities under various conditions, and/or enhanced chelator stability. In addition, the metalloprotease polypeptides of the present invention find use in cleaning compositions that do not include detergents, again either alone or in combination with builders and stabilizers.

In some embodiments of the present invention, the cleaning compositions comprise at least one metalloprotease polypeptide of the present invention at a level from about 0.00001% to about 10% by weight of the composition and the balance (e.g., about 99.999% to about 90.0%) comprising cleaning adjunct materials by weight of composition. In some other embodiments of the present invention, the cleaning compositions of the present invention comprises at least one metalloprotease polypeptide at a level of about 0.0001% to about 10%, about 0.001% to about 5%, about 0.001% to about 2%, about 0.005% to about 0.5% by weight of the composition and the balance of the cleaning composition (e.g., about 99.9999% to about 90.0%, about 99.999% to about 98%, about 99.995% to about 99.5% by weight) comprising cleaning adjunct materials.

In some embodiments, the cleaning compositions of the present invention comprise one or more additional detergent enzymes, which provide cleaning performance and/or fabric care and/or dishwashing benefits. Examples of suitable enzymes include, but are not limited to, acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases, or any combinations or mixtures thereof. In some embodiments, a combination of enzymes is used (i.e., a “cocktail”) comprising conventional applicable enzymes like protease, lipase, cutinase and/or cellulase in conjunction with amylase is used.

In addition to the metalloprotease polypeptides provided herein, any other suitable protease finds use in the compositions of the present invention. Suitable proteases include those of animal, vegetable or microbial origin. In some embodiments, microbial proteases are used. In some embodiments, chemically or genetically modified mutants are included. In some embodiments, the protease is a serine protease, preferably an alkaline microbial protease or a trypsin-like protease. Examples of alkaline proteases include subtilisins, especially those derived from Bacillus (e.g., subtilisin, lentus, amyloliquefaciens, subtilisin Carlsberg, subtilisin 309, subtilisin 147 and subtilisin 168). Additional examples include those mutant proteases described in U.S. Pat. Nos. RE 34,606, 5,955,340, 5,700,676, 6,312,936, and 6,482,628, all of which are incorporated herein by reference. Additional protease examples include, but are not limited to trypsin (e.g., of porcine or bovine origin), and the Fusarium protease described in WO 89/06270. In some embodiments, commercially available protease enzymes that find use in the present invention include, but are not limited to MAXATASE®, MAXACAL™ MAXAPEM™, OPTICLEAN®, OPTIMASE®, PROPERASE®, PURAFECT®, PURAFECT® OXP, PURAMAX™, EXCELLASE™, and PURAFAST™ (Genencor); ALCALASE®, SAVINASE®, PRIMASE®, DURAZYM™, POLARZYME®, OVOZYME®, KANNASE®, LIQUANASE®, NEUTRASE®, RELASE® and ESPERASE® (Novozymes); BLAP™ and BLAP™ variants (Henkel Kommanditgesellschaft auf Aktien, Duesseldorf, Germany), and KAP (B. alkalophilus subtilisin; Kao Corp., Tokyo, Japan). Various proteases are described in WO95/23221, WO 92/21760, WO 09/149200, WO 09/149144, WO 09/149145, WO 11/072099, WO 10/056640, WO 10/056653, WO 11/140364, WO 12/151534, U.S. Pat. Publ. No. 2008/0090747, and U.S. Pat. Nos. 5,801,039, 5,340,735, 5,500,364, 5,855,625, US RE 34,606, 5,955,340, 5,700,676, 6,312,936, and 6,482,628, and various other patents. In some further embodiments, metalloproteases find use in the present invention, including but not limited to the neutral metalloprotease described in WO 07/044993.

In addition, any suitable lipase finds use in the present invention. Suitable lipases include, but are not limited to those of bacterial or fungal origin. Chemically or genetically modified mutants are encompassed by the present invention. Examples of useful lipases include Humicola lanuginosa lipase (See e.g., EP 258 068, and EP 305 216), Rhizomucor miehei lipase (See e.g., EP 238 023), Candida lipase, such as C. antarctica lipase (e.g., the C. antarctica lipase A or B; See e.g., EP 214 761), Pseudomonas lipases such as P. alcaligenes lipase and P. pseudoalcaligenes lipase (See e.g., EP 218 272), P. cepacia lipase (See e.g., EP 331 376), P. stutzeri lipase (See e.g., GB 1,372,034), P. fluorescens lipase, Bacillus lipase (e.g., B. subtilis lipase [Dartois et al., Biochem. Biophys. Acta 1131:253-260 [1993]); B. stearothermophilus lipase [See e.g., JP 64/744992]; and B. pumilus lipase [See e.g., WO 91/16422]).

Furthermore, a number of cloned lipases find use in some embodiments of the present invention, including but not limited to Penicillium camembertii lipase (See, Yamaguchi et al., Gene 103:61-67 [1991]), Geotricum candidum lipase (See, Schimada et al., J. Biochem., 106:383-388 [1989]), and various Rhizopus lipases such as R. delemar lipase (See, Hass et al., Gene 109:117-113 [1991]), a R. niveus lipase (Kugimiya et al., Biosci. Biotech. Biochem. 56:716-719 [1992]) and R. oryzae lipase.

Other types of lipase polypeptide enzymes such as cutinases also find use in some embodiments of the present invention, including but not limited to the cutinase derived from Pseudomonas mendocina (See, WO 88/09367), and the cutinase derived from Fusarium solani pisi (See, WO 90/09446).

Additional suitable lipases include commercially available lipases such as M1 LIPASE™, LUMA FAST™, and LIPOMAX™ (Genencor); LIPEX®, LIPOLASE® and LIPOLASE® ULTRA (Novozymes); and LIPASE P™ “Amano” (Amano Pharmaceutical Co. Ltd., Japan). Various lipases are described in WO2010065455, WO2010107560, WO2011084412, WO2011084417, WO2011084599, WO2011078949, WO2011150157, WO2012137147, WO2013033318, WO2013096653, and U.S. Patent Application No. 61/713,436.

In some embodiments of the present invention, the cleaning compositions of the present invention further comprise lipases at a level from about 0.00001% to about 10% of additional lipase by weight of the composition and the balance of cleaning adjunct materials by weight of composition. In some other embodiments of the present invention, the cleaning compositions of the present invention also comprise lipases at a level of about 0.0001% to about 10%, about 0.001% to about 5%, about 0.001% to about 2%, about 0.005% to about 0.5% lipase by weight of the composition.

In some embodiments of the present invention, any suitable amylase finds use in the present invention. In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present invention, include, but are not limited to α-amylases obtained from B. licheniformis (See e.g., GB 1,296,839). Additional suitable amylases include those found in WO9510603, WO9526397, WO9623874, WO9623873, WO9741213, WO9919467, WO0060060, WO0029560, WO9923211, WO9946399, WO0060058, WO0060059, WO9942567, WO0114532, WO02092797, WO0166712, WO0188107, WO0196537, WO0210355, WO9402597, WO0231124, WO9943793, WO9943794, WO2004113551, WO2005001064, WO2005003311, WO0164852, WO2006063594, WO2006066594, WO2006066596, WO2006012899, WO2008092919, WO2008000825, WO2005018336, WO2005066338, WO2009140504, WO2005019443, WO2010091221, WO2010088447, WO0134784, WO2006012902, WO2006031554, WO2006136161, WO2008101894, WO2010059413, WO2011098531, WO2011080352, WO2011080353, WO2011080354, WO2011082425, WO2011082429, WO2011076123, WO2011087836, WO2011076897, WO94183314, WO9535382, WO9909183, WO9826078, WO9902702, WO9743424, WO9929876, WO9100353, WO9605295, WO9630481, WO9710342, WO2008088493, WO2009149419, WO2009061381, WO2009100102, WO2010104675, WO2010117511, and WO2010115021. Commercially available amylases that find use in the present invention include, but are not limited to DURAMYL®, TERMAMYL®, FUNGAMYL®, STAINZYME®, STAINZYME PLUS®, STAINZYME ULTRA®, and BAN™ (Novozymes), as well as POWERASE™, RAPIDASE® and MAXAMYL® P (Genencor).

In some embodiments of the present invention, the cleaning compositions of the present invention further comprise amylases at a level from about 0.00001% to about 10% of additional amylase by weight of the composition and the balance of cleaning adjunct materials by weight of composition. In some other embodiments of the present invention, the cleaning compositions of the present invention also comprise amylases at a level of about 0.0001% to about 10%, about 0.001% to about 5%, about 0.001% to about 2%, about 0.005% to about 0.5% amylase by weight of the composition.

In some further embodiments, any suitable cellulase finds used in the cleaning compositions of the present invention. Suitable cellulases include, but are not limited to those of bacterial or fungal origin. Chemically or genetically modified mutants are included in some embodiments. Suitable cellulases include, but are not limited to Humicola insolens cellulases (See e.g., U.S. Pat. No. 4,435,307). Especially suitable cellulases are the cellulases having color care benefits (See e.g., EP 0 495 257). Commercially available cellulases that find use in the present include, but are not limited to CELLUZYME®, CAREZYME® (Novozymes), and KAC-500(B)™ (Kao Corporation). In some embodiments, cellulases are incorporated as portions or fragments of mature wild-type or variant cellulases, wherein a portion of the N-terminus is deleted (See e.g., U.S. Pat. No. 5,874,276). Additional suitable cellulases include those found in WO2005054475, WO2005056787, U.S. Pat. No. 7,449,318, and U.S. Pat. No. 7,833,773. In some embodiments, the cleaning compositions of the present invention further comprise cellulases at a level from about 0.00001% to about 10% of additional cellulase by weight of the composition and the balance of cleaning adjunct materials by weight of composition. In some other embodiments of the present invention, the cleaning compositions of the present invention also comprise cellulases at a level of about 0.0001% to about 10%, about 0.001% to about 5%, about 0.001% to about 2%, about 0.005% to about 0.5% cellulase by weight of the composition.

Any mannanase suitable for use in detergent compositions also finds use in the present invention. Suitable mannanases include, but are not limited to those of bacterial or fungal origin. Chemically or genetically modified mutants are included in some embodiments. Various mannanases are known which find use in the present invention (See e.g., U.S. Pat. No. 6,566,114, U.S. Pat. No. 6,602,842, and U.S. Pat. No. 6,440,991, all of which are incorporated herein by reference). Commercially available mannanases that find use in the present invention include, but are not limited to MANNASTAR®, PURABRITE™, and MANNAWAY®. In some embodiments, the cleaning compositions of the present invention further comprise mannanases at a level from about 0.00001% to about 10% of additional mannanase by weight of the composition and the balance of cleaning adjunct materials by weight of composition. In some embodiments of the present invention, the cleaning compositions of the present invention also comprise mannanases at a level of about 0.0001% to about 10%, about 0.001% to about 5%, about 0.001% to about 2%, about 0.005% to about 0.5% mannanase by weight of the composition.

In some embodiments, peroxidases are used in combination with hydrogen peroxide or a source thereof (e.g., a percarbonate, perborate or persulfate) in the compositions of the present invention. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See e.g., WO 94/12621 and WO 95/01426). Suitable peroxidases/oxidases include, but are not limited to those of plant, bacterial or fungal origin. Chemically or genetically modified mutants are included in some embodiments. In some embodiments, the cleaning compositions of the present invention further comprise peroxidase and/or oxidase enzymes at a level from about 0.00001% to about 10% of additional peroxidase and/or oxidase by weight of the composition and the balance of cleaning adjunct materials by weight of composition. In some other embodiments of the present invention, the cleaning compositions of the present invention also comprise, peroxidase and/or oxidase enzymes at a level of about 0.0001% to about 10%, about 0.001% to about 5%, about 0.001% to about 2%, about 0.005% to about 0.5% peroxidase and/or oxidase enzymes by weight of the composition.

In some embodiments, additional enzymes find use, including but not limited to perhydrolases (See e.g., WO 05/056782). In addition, in some embodiments, mixtures of the above mentioned enzymes are encompassed herein, in particular one or more additional protease, amylase, lipase, mannanase, and/or at least one cellulase. Indeed, it is contemplated that various mixtures of these enzymes will find use in the present invention. It is also contemplated that the varying levels of the metalloprotease polypeptide (s) and one or more additional enzymes may both independently range to about 10%, the balance of the cleaning composition being cleaning adjunct materials. The specific selection of cleaning adjunct materials are readily made by considering the surface, item, or fabric to be cleaned, and the desired form of the composition for the cleaning conditions during use (e.g., through the wash detergent use).

Examples of suitable cleaning adjunct materials include, but are not limited to, surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, fabric softeners, carriers, hydrotropes, processing aids, solvents, pigments, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the metalloprotease polypeptides of the present invention in the cleaning compositions, then suitable methods of keeping the cleaning adjunct materials and the protease(s) separated (i.e., not in contact with each other) until combination of the two components is appropriate are used. Such separation methods include any suitable method known in the art (e.g., gelcaps, encapsulation, tablets, physical separation, etc.).

In some embodiments, an effective amount of one or more metalloprotease polypeptide (s) provided herein is included in compositions useful for cleaning a variety of surfaces in need of proteinaceous stain removal. Such cleaning compositions include cleaning compositions for such applications as cleaning hard surfaces, fabrics, and dishes. Indeed, in some embodiments, the present invention provides fabric cleaning compositions, while in other embodiments, the present invention provides non-fabric cleaning compositions. Notably, the present invention also provides cleaning compositions suitable for personal care, including oral care (including dentrifices, toothpastes, mouthwashes, etc., as well as denture cleaning compositions), skin, and hair cleaning compositions. It is intended that the present invention encompass detergent compositions in any form (i.e., liquid, granular, bar, semi-solid, gels, emulsions, tablets, capsules, etc.).

By way of example, several cleaning compositions wherein the metalloprotease polypeptides of the present invention find use are described in greater detail below. In some embodiments in which the cleaning compositions of the present invention are formulated as compositions suitable for use in laundry machine washing method(s), the compositions of the present invention preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials). The compositions of the present invention also find use in detergent additive products in solid or liquid form. Such additive products are intended to supplement and/or boost the performance of conventional detergent compositions and can be added at any stage of the cleaning process. In some embodiments, the density of the laundry detergent compositions herein ranges from about 400 to about 1200 g/liter, while in other embodiments, it ranges from about 500 to about 950 g/liter of composition measured at 20° C.

In embodiments formulated as compositions for use in manual dishwashing methods, the compositions of the invention preferably contain at least one surfactant and preferably at least one additional cleaning adjunct material selected from organic polymeric compounds, suds enhancing agents, group II metal ions, solvents, hydrotropes and additional enzymes.

In some embodiments, various cleaning compositions such as those provided in U.S. Pat. No. 6,605,458, find use with the metalloprotease polypeptides of the present invention. Thus, in some embodiments, the compositions comprising at least one metalloprotease polypeptide of the present invention is a compact granular fabric cleaning composition, while in other embodiments, the composition is a granular fabric cleaning composition useful in the laundering of colored fabrics, in further embodiments, the composition is a granular fabric cleaning composition which provides softening through the wash capacity, in additional embodiments, the composition is a heavy duty liquid fabric cleaning composition. In some embodiments, the compositions comprising at least one metalloprotease polypeptide of the present invention are fabric cleaning compositions such as those described in U.S. Pat. Nos. 6,610,642 and 6,376,450. In addition, the metalloprotease polypeptides of the present invention find use in granular laundry detergent compositions of particular utility under European or Japanese washing conditions (See e.g., U.S. Pat. No. 6,610,642).

In some alternative embodiments, the present invention provides hard surface cleaning compositions comprising at least one metalloprotease polypeptide provided herein. Thus, in some embodiments, the compositions comprising at least one metalloprotease polypeptide of the present invention is a hard surface cleaning composition such as those described in U.S. Pat. Nos. 6,610,642, 6,376,450, and 6,376,450.

In yet further embodiments, the present invention provides dishwashing compositions comprising at least one metalloprotease polypeptide provided herein. Thus, in some embodiments, the compositions comprising at least one metalloprotease polypeptide of the present invention is a hard surface cleaning composition such as those in U.S. Pat. Nos. 6,610,642 and 6,376,450. In some still further embodiments, the present invention provides dishwashing compositions comprising at least one metalloprotease polypeptide provided herein. In some further embodiments, the compositions comprising at least one metalloprotease polypeptide of the present invention comprise oral care compositions such as those in U.S. Pat. Nos. 6,376,450, and 6,376,450. The formulations and descriptions of the compounds and cleaning adjunct materials contained in the aforementioned U.S. Pat. Nos. 6,376,450, 6,605,458, 6,605,458, and 6,610,642, find use with the metalloprotease polypeptides provided herein.

The cleaning compositions of the present invention are formulated into any suitable form and prepared by any process chosen by the formulator, non-limiting examples of which are described in U.S. Pat. Nos. 5,879,584, 5,691,297, 5,574,005, 5,569,645, 5,565,422, 5,516,448, 5,489,392, and 5,486,303, all of which are incorporated herein by reference. When a low pH cleaning composition is desired, the pH of such composition is adjusted via the addition of a material such as monoethanolamine or an acidic material such as HCl.

In some embodiments, the cleaning compositions of the present invention can be formulated to have an alkaline pH under wash conditions, such as a pH of from about 8.0 to about 12.0, or from about 8.5 to about 11.0, or from about 9.0 to about 11.0. In some embodiments, the cleaning compositions of the present invention can be formulated to have a neutral pH under wash conditions, such as a pH of from about 5.0 to about 8.0, or from about 5.5 to about 8.0, or from about 6.0 to about 8.0, or from about 6.0 to about 7.5. In some embodiments, the neutral pH conditions can be measured when the cleaning composition is dissolved 1:100 (wt:wt) in de-ionised water at 20° C., measured using a conventional pH meter.

While not essential for the purposes of the present invention, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to the metalloprotease polypeptides of the present invention. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812, and 6,326,348, incorporated by reference. The aforementioned adjunct ingredients may constitute the balance of the cleaning compositions of the present invention.

In some embodiments, the cleaning compositions according to the present invention comprise an acidifying particle or an amino carboxylic builder. Examples of an amino carboxylic builder include aminocarboxylic acids, salts and derivatives thereof. In some embodiment, the amino carboxylic builder is an aminopolycarboxylic builder, such as glycine-N,N-diacetic acid or derivative of general formula MOOC—CHR—N(CH₂COOM)₂ where R is 12 alkyl and M is alkali metal. In some embodiments, the amino carboxylic builder can be methylglycine diacetic acid (MGDA), GLDA (glutamic-N,N-diacetic acid), iminodisuccinic acid (IDS), carboxymethyl inulin and salts and derivatives thereof, aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl)aspartic acid (SEAS), N-(2-sulfomethyl)glutamic acid (SMGL), N-(2-sulfoethyl) glutamic acid (SEGL), IDS (iminodiacetic acid) and salts and derivatives thereof such as N-methyliminodiacetic acid (MIDA), alpha-alanine-N,N-diacetic acid (alpha-ALDA), serine-N,N-diacetic acid (SEDA), isoserine-N,Ndiacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts and derivative thereof. In some embodiments, the acidifying particle has a weight geometric mean particle size of from about 400μ to about 1200μ and a bulk density of at least 550 g/L. In some embodiments, the acidifying particle comprises at least about 5% of the builder.

In some embodiments, the acidifying particle can comprise any acid, including organic acids and mineral acids. Organic acids can have one or two carboxyls and in some instances up to 15 carbons, especially up to 10 carbons, such as formic, acetic, propionic, capric, oxalic, succinic, adipic, maleic, fumaric, sebacic, malic, lactic, glycolic, tartaric and glyoxylic acids. In some embodiments, the acid is citric acid. Mineral acids include hydrochloric and sulphuric acid. In some instances, the acidifying particle of the invention is a highly active particle comprising a high level of amino carboxylic builder. Sulphuric acid has been found to further contribute to the stability of the final particle.

In some embodiments, the cleaning compositions according to the present invention comprise at least one surfactant and/or a surfactant system wherein the surfactant is selected from nonionic surfactants, anionic surfactants, cationic surfactants, ampholytic surfactants, zwitterionic surfactants, semi-polar nonionic surfactants and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions the acidic contents. In some embodiments, the surfactant is present at a level of from about 0.1% to about 60%, while in alternative embodiments the level is from about 1% to about 50%, while in still further embodiments the level is from about 5% to about 40%, by weight of the cleaning composition.

In some embodiments, the cleaning compositions of the present invention comprise one or more detergent builders or builder systems. In some embodiments incorporating at least one builder, the cleaning compositions comprise at least about 1%, from about 3% to about 60% or even from about 5% to about 40% builder by weight of the cleaning composition. Builders include, but are not limited to, the alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present invention.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present invention, including those known in the art (See e.g., EP 2 100 949).

In some embodiments, builders for use herein include phosphate builders and non-phosphate builders. In some embodiments, the builder is a phosphate builder. In some embodiments, the builder is a non-phosphate builder. If present, builders are used in a level of from 0.1% to 80%, or from 5 to 60%, or from 10 to 50% by weight of the composition. In some embodiments the product comprises a mixture of phosphate and non-phosphate builders. Suitable phosphate builders include mono-phosphates, di-phosphates, tri-polyphosphates or oligomeric-poylphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition of the invention. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include the ammonium and/or alkali metal salts, i.e. the lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

In some embodiments, the cleaning compositions of the present invention contain at least one chelating agent. Suitable chelating agents include, but are not limited to copper, iron and/or manganese chelating agents and mixtures thereof. In embodiments in which at least one chelating agent is used, the cleaning compositions of the present invention comprise from about 0.1% to about 15% or even from about 3.0% to about 10% chelating agent by weight of the subject cleaning composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present invention. In some embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some embodiments, the anti-redeposition agent is a non-ionic surfactant as known in the art (See e.g., EP 2 100 949). In some embodiments, the non-ionic surfactant can be ethoxylated nonionic surfactants, epoxy-capped poly(oxyalkylated) alcohols and amine oxides surfactants.

In some embodiments, the cleaning compositions of the present invention include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. In embodiments in which at least one dye transfer inhibiting agent is used, the cleaning compositions of the present invention comprise from about 0.0001% to about 10%, from about 0.01% to about 5%, or even from about 0.1% to about 3% by weight of the cleaning composition.

In some embodiments, silicates are included within the compositions of the present invention. In some such embodiments, sodium silicates (e.g., sodium disilicate, sodium metasilicate, and crystalline phyllosilicates) find use. In some embodiments, silicates are present at a level of from about 1% to about 20%. In some embodiments, silicates are present at a level of from about 5% to about 15% by weight of the composition.

In some still additional embodiments, the cleaning compositions of the present invention also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts, such as calcium formate. It is contemplated that various techniques for enzyme stabilization will find use in the present invention. For example, in some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), nickel (II), and oxovanadium (IV). Chlorides and sulfates also find use in some embodiments of the present invention. Examples of suitable oligosaccharides and polysaccharides (e.g., dextrins) are known in the art (See e.g., WO 07/145964). In some embodiments, reversible protease inhibitors also find use, such as boron-containing compounds (e.g., borate, 4-formyl phenyl boronic acid) and/or a tripeptide aldehyde find use to further improve stability, as desired.

In some embodiments, bleaches, bleach activators and/or bleach catalysts are present in the compositions of the present invention. In some embodiments, the cleaning compositions of the present invention comprise inorganic and/or organic bleaching compound(s). Inorganic bleaches include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present invention (See e.g., EP 2 100 949).

In some embodiments, bleach activators are used in the compositions of the present invention. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxoycarboxylic acids having preferably from about 1 to about 10 carbon atoms, in particular from about 2 to about 4 carbon atoms, and/or optionally substituted perbenzoic acid. Additional bleach activators are known in the art and find use in the present invention (See e.g., EP 2 100 949).

In addition, in some embodiments and as further described herein, the cleaning compositions of the present invention further comprise at least one bleach catalyst. In some embodiments, the manganese triazacyclononane and related complexes find use, as well as cobalt, copper, manganese, and iron complexes. Additional bleach catalysts find use in the present invention (See e.g., U.S. Pat. Nos. 4,246,612, 5,227,084, 4,810410, WO 99/06521, and EP 2 100 949).

In some embodiments, the cleaning compositions of the present invention contain one or more catalytic metal complexes. In some embodiments, a metal-containing bleach catalyst finds use. In some embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See e.g., U.S. Pat. No. 4,430,243). In some embodiments, the cleaning compositions of the present invention are catalyzed by means of a manganese compound. Such compounds and levels of use are well known in the art (See e.g., U.S. Pat. No. 5,576,282). In additional embodiments, cobalt bleach catalysts find use in the cleaning compositions of the present invention. Various cobalt bleach catalysts are known in the art (See e.g., U.S. Pat. Nos. 5,597,936 and 5,595,967) and are readily prepared by known procedures.

In some additional embodiments, the cleaning compositions of the present invention include a transition metal complex of a macropolycyclic rigid ligand (MRL). As a practical matter, and not by way of limitation, in some embodiments, the compositions and cleaning processes provided by the present invention are adjusted to provide on the order of at least one part per hundred million of the active MRL species in the aqueous washing medium, and in some embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, transition-metals in the instant transition-metal bleach catalyst include, but are not limited to manganese, iron and chromium. MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See e.g., WO 2000/32601, and U.S. Pat. No. 6,225,464).

In some embodiments, the cleaning compositions of the present invention comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2 100 949, WO 9426860 and WO 94/26859). In some embodiments, the metal care agent is a zinc salt. In some further embodiments, the cleaning compositions of the present invention comprise from about 0.1% to about 5% by weight of one or more metal care agent.

In some embodiments, the cleaning composition is a high density liquid (HDL) composition having a variant metalloprotease polypeptide protease. The HDL liquid laundry detergent can comprise a detersive surfactant (10%-40%) comprising anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof); and optionally non-ionic surfactant (selected from a group of linear or branched or random chain, substituted or unsubstituted alkyl alkoxylated alcohol, for example a C₈-C₁₈ alkyl ethoxylated alcohol and/or C₆-C₁₂ alkyl phenol alkoxylates), optionally wherein the weight ratio of anionic detersive surfactant (with a hydrophilic index (HIc) of from 6.0 to 9) to non-ionic detersive surfactant is greater than 1:1.

The composition can comprise optionally, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C₁-C₆ carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂₅ alkyl group, polypropylene, polybutylene, vinyl ester of a saturated C—C₆ mono-carboxylic acid, C₁-C₆ alkyl ester of acrylic or methacrylic acid, and mixtures thereof.

The composition can comprise additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100, SRN170, SRN240, SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixtures thereof) and polymeric carboxylate (such as maleate/acrylate random copolymer or polyacrylate homopolymer).

The composition can further comprise saturated or unsaturated fatty acid, preferably saturated or unsaturated C₁₂-C₂₄ fatty acid (0 wt % to 10 wt %); deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydroxyethyl cellulose, cationic starch, cationic polyacrylamides, and mixtures thereof.

The composition can further comprise dye transfer inhibiting agents examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof.

The composition can further comprise enzymes (0.01 wt % active enzyme to 0.03 wt % active enzyme) selected from a group of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases, and any mixture thereof. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition can further comprise silicone or fatty-acid based suds suppressors; heuing dyes, calcium and magnesium cations, visual signaling ingredients, anti-foam (0.001 wt % to about 4.0 wt %), and/or structurant/thickener (0.01 wt % to 5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

Suitable detersive surfactants also include cationic detersive surfactants (selected from a group of alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

The composition can be any liquid form, for example a liquid or gel form, or any combination thereof. The composition may be in any unit dose form, for example a pouch.

In some embodiments, the cleaning composition is a high density powder (HDD) composition having a variant metalloprotease polypeptide protease. The HDD powder laundry detergent can comprise a detersive surfactant including anionic detersive surfactants (selected from a group of linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (selected from a group of linear or branched or random chain, substituted or unsubstituted C₈-C₁₈ alkyl ethoxylates, and/or C₆-C₁₂ alkyl phenol alkoxylates), cationic detersive surfactants (selected from a group of alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (selected from a group of alkanolamine sulpho-betaines); ampholytic surfactants; semi-polar non-ionic surfactants and mixtures thereof; builders (phosphate free builders [for example zeolite builders examples of which include zeolite A, zeolite X, zeolite P and zeolite MAP in the range of 0 wt % to less than 10 wt %]; phosphate builders [examples of which include sodium tri-polyphosphate in the range of 0 wt % to less than 10 wt %]; citric acid, citrate salts and nitrilotriacetic acid or salt thereof in the range of less than 15 wt %); silicate salt (sodium or potassium silicate or sodium meta-silicate in the range of 0 wt % to less than 10 wt %, or layered silicate (SKS-6)); carbonate salt (sodium carbonate and/or sodium bicarbonate in the range of 0 wt % to less than 10 wt %); and bleaching agents (photobleaches, examples of which include sulfonated zinc phthalocyanines, sulfonated aluminum phthalocyanines, xanthenes dyes, and mixtures thereof; hydrophobic or hydrophilic bleach activators (examples of which include dodecanoyl oxybenzene sulfonate, decanoyl oxybenzene sulfonate, decanoyl oxybenzoic acid or salts thereof, 3,5,5-trimethyl hexanoyl oxybenzene sulfonate, tetraacetyl ethylene diamine-TAED, and nonanoyloxybenzene sulfonate-NOBS, nitrile quats, and mixtures thereof; hydrogen peroxide; sources of hydrogen peroxide (inorganic perhydrate salts examples of which include mono or tetra hydrate sodium salt of perborate, percarbonate, persulfate, perphosphate, or persilicate); preformed hydrophilic and/or hydrophobic peracids (selected from a group consisting of percarboxylic acids and salts, percarbonic acids and salts, perimidic acids and salts, peroxymonosulfuric acids and salts) & mixtures thereof and/or bleach catalyst (such as imine bleach boosters examples of which include iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof; metal-containing bleach catalyst for example copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephos [symbol] phonic acid) and water-soluble salts thereof).

The composition can further comprise enzymes selected from a group of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, glucose oxidases, hemicellulases, hyaluronidases, keratinases, laccases, lactases, ligninases, lipases, lipoxygenases, mannanases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases and any mixture thereof.

The composition can further comprise additional detergent ingredients including perfume microcapsules, starch encapsulated perfume accord, hueing agents, additional polymers including fabric integrity and cationic polymers, dye lock ingredients, fabric-softening agents, brighteners (for example C.I. Fluorescent brighteners), flocculating agents, chelating agents, alkoxylated polyamines, fabric deposition aids, and/or cyclodextrin.

In some embodiments, the cleaning composition is an automatic dishwashing (ADW) detergent composition having a metalloprotease of the present invention. The ADW detergent composition can comprise two or more non-ionic surfactants selected from a group of ethoxylated non-ionic surfactants, alcohol alkoxylated surfactants, epoxy-capped poly(oxyalkylated) alcohols, or amine oxide surfactants present in amounts from 0 to 10% by weight; builders in the range of 5-60% comprising either phosphate (mono-phosphates, di-phosphates, tri-polyphosphates or oligomeric-poylphosphates, preferred sodium tripolyphosphate-STPP or phosphate-free builders [amino acid based compounds, examples of which include MGDA (methyl-glycine-diacetic acid), and salts and derivatives thereof, GLDA (glutamic-N,Ndiacetic acid) and salts and derivatives thereof, IDS (iminodisuccinic acid) and salts and derivatives thereof, carboxy methyl inulin and salts and derivatives thereof and mixtures thereof, nitrilotriacetic acid (NTA), diethylene triamine penta acetic acid (DTPA), B-alaninediacetic acid (B-ADA) and their salts], homopolymers and copolymers of polycarboxylic acids and their partially or completely neutralized salts, monomeric polycarboxylic acids and hydroxycarboxylic acids and their salts in the range of 0.5% to 50% by weight; sulfonated/carboxylated polymers (provide dimensional stability to the product) in the range of about 0.1% to about 50% by weight; drying aids in the range of about 0.1% to about 10% by weight (selected from polyesters, especially anionic polyesters optionally together with further monomers with 3 to 6 functionalities which are conducive to polycondensation, specifically acid, alcohol or ester functionalities, polycarbonate-, polyurethane- and/or polyurea-polyorganosiloxane compounds or precursor compounds thereof of the reactive cyclic carbonate and urea type); silicates in the range from about 1% to about 20% by weight (sodium or potassium silicates for example sodium disilicate, sodium meta-silicate and crystalline phyllosilicates); bleach-inorganic (for example perhydrate salts such as perborate, percarbonate, perphosphate, persulfate and persilicate salts) and organic (for example organic peroxyacids including diacyl and tetraacylperoxides, especially diperoxydodecanedioc acid, diperoxytetradecanedioc acid, and diperoxyhexadecanedioc acid); bleach activators—organic peracid precursors in the range from about 0.1% to about 10% by weight; bleach catalysts (selected from manganese triazacyclononane and related complexes, Co, Cu, Mn and Fe bispyridylamine and related complexes, and pentamine acetate cobalt(III) and related complexes); metal care agents in the range from about 0.1% to 5% by weight (selected from benzatriazoles, metal salts and complexes, and/or silicates); enzymes in the range from about 0.01 to 5.0 mg of active enzyme per gram of automatic dishwashing detergent composition (acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases, and any mixture thereof); and enzyme stabilizer components (selected from oligosaccharides, polysaccharides and inorganic divalent metal salts).

Representative detergent formulations that beneficially include a metalloprotease polypeptide of the present invention include the detergent formulations found in WO2013063460, pages 78-152, and in particular the tables of pages 94 to 152 are hereby incorporated by reference. The metalloproteases are normally incorporated into the detergent composition at a level of from 0.000001% to 5% of enzyme protein by weight of the composition, or from 0.00001% to 2%, or from 0.0001% to 1%, or from 0.001% to 0.75% of enzyme protein by weight of the composition.

### Metalloprotease Polypeptides of the Present Invention for Use in Animal Feed

In a further aspect of the invention, the metalloprotease polypeptides of the present invention can be used as a component of an animal feed composition, animal feed additive and/or pet food comprising a metalloprotease and variants thereof. The present invention further relates to a method for preparing such an animal feed composition, animal feed additive composition and/or pet food comprising mixing the metalloprotease polypeptide with one or more animal feed ingredients and/or animal feed additive ingredients and/or pet food ingredients. Furthermore, the present invention relates to the use of the metalloprotease polypeptide in the preparation of an animal feed composition and/or animal feed additive composition and/or pet food.

The term “animal” includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS) (particularly corn based Distillers Dried Grain Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

### Metalloprotease Polypeptides of the Present Invention for Use in Textile Desizing

Also contemplated are compositions and methods of treating fabrics (e.g., to desize a textile) using a metalloprotease polypeptide of the present invention. Fabric-treating methods are well known in the art (see, e.g., U.S. Pat. No. 6,077,316). For example, the feel and appearance of a fabric can be improved by a method comprising contacting the fabric with a metalloprotease in a solution. The fabric can be treated with the solution under pressure.

A metalloprotease of the present invention can be applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. A metalloprotease of the present invention can be applied during or after the weaving to remove these sizing starch or starch derivatives. After weaving, the metalloprotease can be used to remove the size coating before further processing the fabric to ensure a homogeneous and wash-proof result.

A metalloprotease of the present invention can be used alone or with other desizing chemical reagents and/or desizing enzymes to desize fabrics, including cotton-containing fabrics, as detergent additives, e.g., in aqueous compositions. An amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of proteolytic enzymes to provide softness to the fabric and make the cotton more accessible to the subsequent enzymatic finishing steps. The metalloprotease can be used in methods of finishing denim garments (e.g., a “bio-stoning process”), enzymatic desizing and providing softness to fabrics, and/or finishing process.

### Metalloprotease Polypeptides of the Present Invention for Use in Paper Pulp Bleaching

The metalloprotease polypeptides described herein find further use in the enzyme aided bleaching of paper pulps such as chemical pulps, semi-chemical pulps, kraft pulps, mechanical pulps or pulps prepared by the sulfite method. In general terms, paper pulps are incubated with a metalloprotease polypeptide of the present invention under conditions suitable for bleaching the paper pulp.

In some embodiments, the pulps are chlorine free pulps bleached with oxygen, ozone, peroxide or peroxyacids. In some embodiments, the metalloprotease polypeptides are used in enzyme aided bleaching of pulps produced by modified or continuous pulping methods that exhibit low lignin contents. In some other embodiments, the metalloprotease polypeptides are applied alone or preferably in combination with xylanase and/or endoglucanase and/or alpha-galactosidase and/or cellobiohydrolase enzymes.

### Metalloprotease Polypeptides of the Present Invention for Use in Protein Degradation

The metalloprotease polypeptides described herein find further use in the enzyme aided removal of proteins from animals and their subsequent degradation or disposal, such as feathers, skin, hair, hide, and the like. In some instances, immersion of the animal carcass in a solution comprising a metalloprotease polypeptide of the present invention can act to protect the skin from damage in comparison to the traditional immersion in scalding water or the defeathering process. In one embodiment, feathers can be sprayed with an isolated metalloprotase polypeptide of the present invention under conditions suitable for digesting or initiating degradation of the plumage. In some embodiments, a metalloprotease of the present invention can be used, as above, in combination with an oxidizing agent.

In some embodiments, removal of the oil or fat associated with raw feathers is assisted by using a metalloprotease polypeptide of the present invention. In some embodiments, the metalloprotease polypeptides are used in compositions for cleaning the feathers as well as to sanitize and partially dehydrate the fibers. In some other embodiments, the metalloprotease polypeptides are applied in a wash solution in combination with 95% ethanol or other polar organic solvent with or without a surfactant at about 0.5% (v/v).

In yet other embodiments, the disclosed metalloprotease polypeptides find use in recovering protein from plumage. In some embodiments, the recovered protein can be subsequently used in animal or fish feed.

### Metalloprotease Polypeptides of the Present Invention for Use in Tissue Debridement

The metalloprotease polypeptides described herein find further use in the enzyme aided debridement of tissue. This involves the removal of dead or damaged tissue, for example, removal from wounds to aid in healing.

### Metalloprotease Polypeptides of the Present Invention for Use in Tissue Culture

The metalloprotease polypeptides described herein find further use in tissue culture. In particular, metalloproteases of the present invention can be used to suspend or resuspend cells adherent to a cell culture wall, such as during the process of harvesting cells. Metalloproteases of the present invention can be used to cleave protein bonds between cultured cells and the dish, allowing cells to become suspended in solution.

### Metalloprotease Polypeptides of the Present Invention for Use in Leather Processing

The metalloprotease polypeptides described herein find further use in leather processing by removing hair from animal hides, soaking, degreasing, or bating, which is a process involving degradation of non-structural proteins during leather making.

### EXPERIMENTAL

The claimed invention is described in further detail in the following examples which are not in any way intended to limit the scope of the invention as claimed.

### Example 1

### Crystallization and Structure Determination of PehPro1 Metalloprotease

The metalloprotease PehPro1, encoded by a Paenibacillus ehimensis strain was crystallized using the hanging drop method from a solution of protein stock at a concentration of 27.9 mg/mL in 20 mM Tris pH 8.5+0.10M Sodium chloride+1 mM Calcium chloride. Aliquots of 2 μL of the protein stock and 2 μL of the crystallization solution were mixed on a plastic coverslip and inverted and sealed on a chamber containing 15-25% Polyethylene Glycol 8000+50 mM Potassium phosphate monobasic+0.10M HEPES pH 7.5 in a Linbro 6×4 culture plate.

Crystals grew in the space group C 2 2 2₁ with unit cell dimensions; a=58.814 Å, b=194.346 Å, and c=138.355 Å. Data were collected on native crystal to 2.79 Å resolution and the structure of PehPro1 was determined by molecular replacement using a related protein (pdb ID 1ESP) as the phasing model. The statistics of data collected are presented in Table 1.1.

TABLE 1.1
Statistics of PehPro1 Data collection

| Wavelength | 1.54 | Å |
|---|---|---|
| Space group | C222 | |
| Molecules in asymmetric unit | 2 | |
| Unit cell dimensions | a = 58.81 Å, b = 194.35 Å and | |
| | c = 138.36 Å | |
| Resolution | 32.6-2.79 | Å |
| Unique reflections | 19103 | |
| Multiplicity | 17 | (11*) |
| Completeness | 99.33% | (92%*) |
| R_{merge} | 0.08 | (0.30*) |
| I/σ_{I} | 17 | (8*) |

*Value in parenthesis is that of the outermost shell of data

The model was fitted in the resulting electron density using the program COOT (Emsley, P et al Acta Cryst. D66 486-501 (2010)). After fitting and refitting adjustments, the coordinates were refined using the REFMAC program with standard defaults in the CCP4 software suite. The statistics of the current model are presented in Table 1.2.

TABLE 1.2
Statistics of the refined model

| | |
|---|---|
| R work | 0.19 |
| R free | 0.25 |
| No. protein residues | 304 |
| No. atoms | 4742 |
| rmsd Bond lengths | 0.013 Å |
| rmsd bond angles | 1.6° |

### Example 2

### PehPro1 Crystal Structure Details

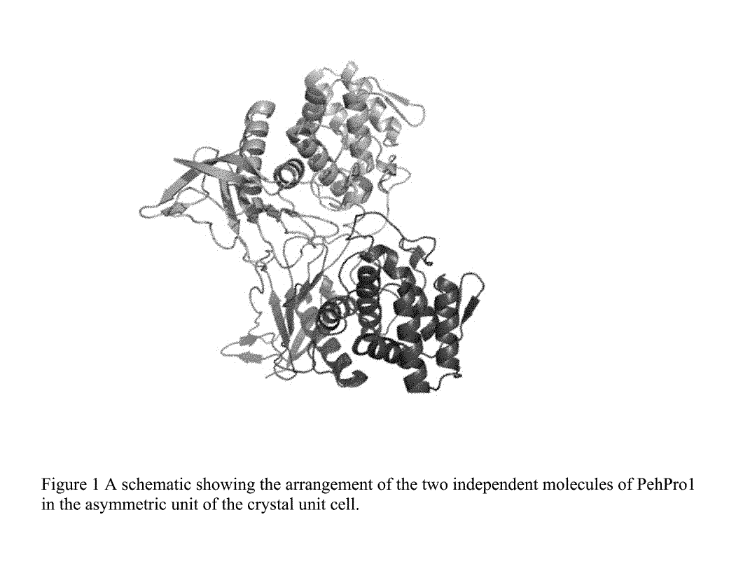

The structure of PehPro1 consists of a dimer of two equivalent molecules. Electron density was available for residues 1-304 of each monomer. Each model was fitted to contiguous density. The overall dimer arrangement is presented in FIG. 1. The residues forming the active site regions include the catalytic residues; His135, Glu136, His139 and Glu159 (numbering based on PehPro1 mature sequence) forming the characteristic zinc metal binding site, along with other residues forming the substrate binding pocket are conserved between the PehPro1 and Thermolysin structures (Matthews, B. W., Weaver, L. H., Kester, W. R., The Conformation of Thermolysin, (1974) J. Biol. Chem. 249: 8030; Dahlquist, F. W., Long, J. W., Bigbee, W. L., Role of Calcium in the Thermal Stability of Thermolysin, (1976) Biochemistry 15: 1103).

The coordinates for this molecule are listed below.

| | | | | | | | | | | | | |
|---|---|---|---|---|---|---|---|---|---|---|---|---|
| ATOM | 1 | N | ALA | A | 1 | −17.159 | −64.455 | −9.260 | 1.00 | 39.68 | A | N |
| ATOM | 2 | CA | ALA | A | 1 | −18.338 | −64.728 | −10.130 | 1.00 | 33.72 | A | C |
| ATOM | 3 | CB | ALA | A | 1 | −19.640 | −64.092 | −9.687 | 1.00 | 32.16 | A | C |
| ATOM | 4 | C | ALA | A | 1 | −17.839 | −63.991 | −11.323 | 1.00 | 30.98 | A | C |
| ATOM | 5 | O | ALA | A | 1 | −17.083 | −63.038 | −11.132 | 1.00 | 31.11 | A | O |
| ATOM | 6 | N | THR | A | 2 | −18.230 | −64.375 | −12.537 | 1.00 | 27.24 | A | N |
| ATOM | 7 | CA | THR | A | 2 | −17.655 | −63.718 | −13.709 | 1.00 | 23.73 | A | C |
| ATOM | 8 | CB | THR | A | 2 | −16.933 | −64.722 | −14.609 | 1.00 | 23.24 | A | C |
| ATOM | 9 | OG1 | THR | A | 2 | −16.035 | −65.475 | −13.800 | 1.00 | 23.09 | A | O |
| ATOM | 10 | CG2 | THR | A | 2 | −16.117 | −64.026 | −15.659 | 1.00 | 22.84 | A | C |
| ATOM | 11 | C | THR | A | 2 | −18.690 | −62.894 | −14.454 | 1.00 | 22.06 | A | C |
| ATOM | 12 | O | THR | A | 2 | −19.503 | −63.421 | −15.169 | 1.00 | 22.29 | A | O |
| ATOM | 13 | N | GLY | A | 3 | −18.652 | −61.581 | −14.259 | 1.00 | 21.36 | A | N |
| ATOM | 14 | CA | GLY | A | 3 | −19.512 | −60.660 | −14.981 | 1.00 | 20.30 | A | C |
| ATOM | 15 | C | GLY | A | 3 | −18.962 | −60.326 | −16.364 | 1.00 | 20.22 | A | C |
| ATOM | 16 | O | GLY | A | 3 | −17.744 | −60.439 | −16.667 | 1.00 | 18.32 | A | O |
| ATOM | 17 | N | THR | A | 4 | −19.885 | −59.870 | −17.199 | 1.00 | 20.55 | A | N |
| ATOM | 18 | CA | THR | A | 4 | −19.612 | −59.576 | −18.593 | 1.00 | 20.58 | A | C |
| ATOM | 19 | CB | THR | A | 4 | −19.948 | −60.825 | −19.426 | 1.00 | 20.39 | A | C |
| ATOM | 20 | OG1 | THR | A | 4 | −19.095 | −60.904 | −20.570 | 1.00 | 21.45 | A | O |
| ATOM | 21 | CG2 | THR | A | 4 | −21.404 | −60.866 | −19.797 | 1.00 | 20.52 | A | C |
| ATOM | 22 | C | THR | A | 4 | −20.371 | −58.290 | −18.979 | 1.00 | 20.82 | A | C |
| ATOM | 23 | O | THR | A | 4 | −21.362 | −57.946 | −18.344 | 1.00 | 20.84 | A | O |
| ATOM | 24 | N | GLY | A | 5 | −19.870 | −57.532 | −19.955 | 1.00 | 22.14 | A | N |
| ATOM | 25 | CA | GLY | A | 5 | −20.474 | −56.214 | −20.307 | 1.00 | 21.26 | A | C |
| ATOM | 26 | C | GLY | A | 5 | −19.683 | −55.446 | −21.353 | 1.00 | 21.65 | A | C |
| ATOM | 27 | O | GLY | A | 5 | −18.525 | −55.770 | −21.629 | 1.00 | 21.95 | A | O |
| ATOM | 28 | N | LYS | A | 6 | −20.293 | −54.433 | −21.958 | 1.00 | 23.22 | A | N |
| ATOM | 29 | CA | LYS | A | 6 | −19.599 | −53.661 | −22.999 | 1.00 | 23.98 | A | C |
| ATOM | 30 | CB | LYS | A | 6 | −20.501 | −53.316 | −24.191 | 1.00 | 26.75 | A | C |
| ATOM | 31 | CG | LYS | A | 6 | −21.062 | −54.545 | −24.928 | 1.00 | 31.43 | A | C |
| ATOM | 32 | CD | LYS | A | 6 | −21.426 | −54.362 | −26.429 | 1.00 | 34.80 | A | C |
| ATOM | 33 | CE | LYS | A | 6 | −22.314 | −53.151 | −26.767 | 1.00 | 37.30 | A | C |
| ATOM | 34 | NZ | LYS | A | 6 | −23.750 | −53.306 | −26.350 | 1.00 | 38.51 | A | N |
| ATOM | 35 | C | LYS | A | 6 | −19.019 | −52.412 | −22.379 | 1.00 | 21.52 | A | C |
| ATOM | 36 | O | LYS | A | 6 | −19.655 | −51.799 | −21.527 | 1.00 | 21.20 | A | O |
| ATOM | 37 | N | GLY | A | 7 | −17.806 | −52.049 | −22.801 | 1.00 | 19.41 | A | N |
| ATOM | 38 | CA | GLY | A | 7 | −17.124 | −50.843 | −22.311 | 1.00 | 17.91 | A | C |
| ATOM | 39 | C | GLY | A | 7 | −17.709 | −49.607 | −22.918 | 1.00 | 17.02 | A | C |
| ATOM | 40 | O | GLY | A | 7 | −18.733 | −49.657 | −23.599 | 1.00 | 17.62 | A | O |
| ATOM | 41 | N | VAL | A | 8 | −17.062 | −48.481 | −22.697 | 1.00 | 16.27 | A | N |
| ATOM | 42 | CA | VAL | A | 8 | −17.536 | −47.207 | −23.273 | 1.00 | 15.98 | A | C |
| ATOM | 43 | CB | VAL | A | 8 | −16.608 | −46.061 | −22.849 | 1.00 | 16.24 | A | C |
| ATOM | 44 | CG1 | VAL | A | 8 | −16.843 | −44.785 | −23.665 | 1.00 | 15.97 | A | C |
| ATOM | 45 | CG2 | VAL | A | 8 | −16.770 | −45.827 | −21.354 | 1.00 | 16.69 | A | C |
| ATOM | 46 | C | VAL | A | 8 | −17.580 | −47.271 | −24.800 | 1.00 | 15.88 | A | C |
| ATOM | 47 | O | VAL | A | 8 | −18.502 | −46.760 | −25.430 | 1.00 | 14.79 | A | O |
| ATOM | 48 | N | LEU | A | 9 | −16.564 | −47.928 | −25.366 | 1.00 | 15.93 | A | N |
| ATOM | 49 | CA | LEU | A | 9 | −16.361 | −48.015 | −26.814 | 1.00 | 15.86 | A | C |
| ATOM | 50 | CB | LEU | A | 9 | −14.852 | −48.093 | −27.086 | 1.00 | 15.92 | A | C |
| ATOM | 51 | CG | LEU | A | 9 | −14.126 | −46.852 | −27.579 | 1.00 | 15.70 | A | C |
| ATOM | 52 | CD1 | LEU | A | 9 | −14.696 | −45.602 | −26.965 | 1.00 | 15.39 | A | C |
| ATOM | 53 | CD2 | LEU | A | 9 | −12.636 | −46.998 | −27.283 | 1.00 | 15.95 | A | C |
| ATOM | 54 | C | LEU | A | 9 | −17.023 | −49.217 | −27.493 | 1.00 | 15.36 | A | C |
| ATOM | 55 | O | LEU | A | 9 | −16.577 | −49.596 | −28.592 | 1.00 | 15.14 | A | O |
| ATOM | 56 | N | GLY | A | 10 | −18.010 | −49.842 | −26.831 | 1.00 | 14.23 | A | N |
| ATOM | 57 | CA | GLY | A | 10 | −18.771 | −50.970 | −27.404 | 1.00 | 13.71 | A | C |
| ATOM | 58 | C | GLY | A | 10 | −18.144 | −52.351 | −27.326 | 1.00 | 13.72 | A | C |
| ATOM | 59 | O | GLY | A | 10 | −18.738 | −53.341 | −27.753 | 1.00 | 13.58 | A | O |
| ATOM | 60 | N | ASP | A | 11 | −16.945 | −52.433 | −26.768 | 1.00 | 13.97 | A | N |
| ATOM | 61 | CA | ASP | A | 11 | −16.223 | −53.677 | −26.721 | 1.00 | 14.03 | A | C |
| ATOM | 62 | CB | ASP | A | 11 | −14.715 | −53.447 | −26.716 | 1.00 | 14.10 | A | C |
| ATOM | 63 | CG | ASP | A | 11 | −14.243 | −52.431 | −25.655 | 1.00 | 14.65 | A | C |
| ATOM | 64 | OD1 | ASP | A | 11 | −14.971 | −51.470 | −25.307 | 1.00 | 14.45 | A | O |
| ATOM | 65 | OD2 | ASP | A | 11 | −13.091 | −52.593 | −25.195 | 1.00 | 15.19 | A | O |
| ATOM | 66 | C | ASP | A | 11 | −16.647 | −54.501 | −25.529 | 1.00 | 14.72 | A | C |
| ATOM | 67 | O | ASP | A | 11 | −16.756 | −53.984 | −24.433 | 1.00 | 14.88 | A | O |
| ATOM | 68 | N | THR | A | 12 | −16.844 | −55.802 | −25.763 | 1.00 | 15.81 | A | N |
| ATOM | 69 | CA | THR | A | 12 | −17.317 | −56.754 | −24.758 | 1.00 | 15.88 | A | C |
| ATOM | 70 | CB | THR | A | 12 | −17.962 | −57.966 | −25.452 | 1.00 | 15.68 | A | C |
| ATOM | 71 | OG1 | THR | A | 12 | −19.275 | −57.606 | −25.914 | 1.00 | 15.63 | A | O |
| ATOM | 72 | CG2 | THR | A | 12 | −18.054 | −59.155 | −24.518 | 1.00 | 15.69 | A | C |
| ATOM | 73 | C | THR | A | 12 | −16.171 | −57.235 | −23.884 | 1.00 | 16.48 | A | C |
| ATOM | 74 | O | THR | A | 12 | −15.136 | −57.569 | −24.392 | 1.00 | 16.69 | A | O |
| ATOM | 75 | N | LYS | A | 13 | −16.380 | −57.296 | −22.572 | 1.00 | 17.65 | A | N |
| ATOM | 76 | CA | LYS | A | 13 | −15.333 | −57.703 | −21.603 | 1.00 | 18.19 | A | C |
| ATOM | 77 | CB | LYS | A | 13 | −14.788 | −56.474 | −20.861 | 1.00 | 19.32 | A | C |
| ATOM | 78 | CG | LYS | A | 13 | −13.717 | −55.737 | −21.568 | 1.00 | 20.03 | A | C |
| ATOM | 79 | CD | LYS | A | 13 | −13.757 | −54.265 | −21.245 | 1.00 | 20.24 | A | C |
| ATOM | 80 | CE | LYS | A | 13 | −12.730 | −53.620 | −22.168 | 1.00 | 21.08 | A | C |
| ATOM | 81 | NZ | LYS | A | 13 | −13.089 | −52.212 | −22.399 | 1.00 | 21.72 | A | N |
| ATOM | 82 | C | LYS | A | 13 | −15.855 | −58.583 | −20.482 | 1.00 | 17.16 | A | C |
| ATOM | 83 | O | LYS | A | 13 | −17.047 | −58.515 | −20.130 | 1.00 | 15.92 | A | O |
| ATOM | 84 | N | SER | A | 14 | −14.904 | −59.286 | −19.864 | 1.00 | 16.11 | A | N |
| ATOM | 85 | CA | SER | A | 14 | −15.114 | −60.029 | −18.621 | 1.00 | 16.16 | A | C |
| ATOM | 86 | CB | SER | A | 14 | −14.659 | −61.492 | −18.751 | 1.00 | 15.77 | A | C |
| ATOM | 87 | OG | SER | A | 14 | −15.523 | −62.202 | −19.587 | 1.00 | 15.69 | A | O |
| ATOM | 88 | C | SER | A | 14 | −14.324 | −59.439 | −17.460 | 1.00 | 15.77 | A | C |
| ATOM | 89 | O | SER | A | 14 | −13.254 | −58.842 | −17.641 | 1.00 | 16.38 | A | O |
| ATOM | 90 | N | PHE | A | 15 | −14.833 | −59.712 | −16.263 | 1.00 | 14.76 | A | N |
| ATOM | 91 | CA | PHE | A | 15 | −14.281 | −59.224 | −15.009 | 1.00 | 13.44 | A | C |
| ATOM | 92 | CB | PHE | A | 15 | −14.562 | −57.741 | −14.855 | 1.00 | 12.78 | A | C |
| ATOM | 93 | CG | PHE | A | 15 | −15.901 | −57.329 | −15.375 | 1.00 | 12.44 | A | C |
| ATOM | 94 | CD1 | PHE | A | 15 | −17.004 | −57.402 | −14.588 | 1.00 | 12.46 | A | C |
| ATOM | 95 | CE1 | PHE | A | 15 | −18.248 | −57.033 | −15.066 | 1.00 | 12.45 | A | C |
| ATOM | 96 | CZ | PHE | A | 15 | −18.398 | −56.582 | −16.350 | 1.00 | 12.34 | A | C |
| ATOM | 97 | CE2 | PHE | A | 15 | −17.288 | −56.491 | −17.148 | 1.00 | 12.82 | A | C |
| ATOM | 98 | CD2 | PHE | A | 15 | −16.046 | −56.868 | −16.655 | 1.00 | 12.74 | A | C |
| ATOM | 99 | C | PHE | A | 15 | −14.946 | −59.973 | −13.852 | 1.00 | 13.24 | A | C |
| ATOM | 100 | O | PHE | A | 15 | −15.970 | −60.647 | −14.005 | 1.00 | 12.99 | A | O |
| ATOM | 101 | N | THR | A | 16 | −14.373 | −59.798 | −12.679 | 1.00 | 12.90 | A | N |
| ATOM | 102 | CA | THR | A | 16 | −14.843 | −60.461 | −11.492 | 1.00 | 12.27 | A | C |
| ATOM | 103 | CB | THR | A | 16 | −13.661 | −60.746 | −10.602 | 1.00 | 11.79 | A | C |
| ATOM | 104 | OG1 | THR | A | 16 | −12.628 | −61.323 | −11.424 | 1.00 | 11.21 | A | O |
| ATOM | 105 | CG2 | THR | A | 16 | −14.056 | −61.662 | −9.465 | 1.00 | 11.43 | A | C |
| ATOM | 106 | C | THR | A | 16 | −15.862 | −59.593 | −10.771 | 1.00 | 12.41 | A | C |
| ATOM | 107 | O | THR | A | 16 | −15.728 | −58.359 | −10.686 | 1.00 | 12.07 | A | O |
| ATOM | 108 | N | THR | A | 17 | −16.914 | −60.252 | −10.303 | 1.00 | 12.28 | A | N |
| ATOM | 109 | CA | THR | A | 17 | −17.952 | −59.582 | −9.540 | 1.00 | 12.14 | A | C |
| ATOM | 110 | CB | THR | A | 17 | −19.202 | −59.303 | −10.396 | 1.00 | 11.78 | A | C |
| ATOM | 111 | OG1 | THR | A | 17 | −19.721 | −60.532 | −10.919 | 1.00 | 11.36 | A | O |
| ATOM | 112 | CG2 | THR | A | 17 | −18.838 | −58.371 | −11.537 | 1.00 | 11.82 | A | C |
| ATOM | 113 | C | THR | A | 17 | −18.321 | −60.453 | −8.367 | 1.00 | 12.21 | A | C |
| ATOM | 114 | O | THR | A | 17 | −17.882 | −61.580 | −8.242 | 1.00 | 10.79 | A | O |
| ATOM | 115 | N | THR | A | 18 | −19.156 | −59.908 | −7.515 | 1.00 | 13.29 | A | N |
| ATOM | 116 | CA | THR | A | 18 | −19.482 | −60.584 | −6.301 | 1.00 | 14.74 | A | C |
| ATOM | 117 | CB | THR | A | 18 | −18.921 | −59.785 | −5.103 | 1.00 | 14.74 | A | C |
| ATOM | 118 | OG1 | THR | A | 18 | −17.470 | −59.893 | −5.058 | 1.00 | 14.74 | A | O |
| ATOM | 119 | CG2 | THR | A | 18 | −19.524 | −60.310 | −3.835 | 1.00 | 15.11 | A | C |
| ATOM | 120 | C | THR | A | 18 | −21.010 | −60.819 | −6.232 | 1.00 | 15.79 | A | C |
| ATOM | 121 | O | THR | A | 18 | −21.779 | −59.898 | −6.354 | 1.00 | 14.67 | A | O |
| ATOM | 122 | N | GLN | A | 19 | −21.424 | −62.076 | −6.075 | 1.00 | 17.88 | A | N |
| ATOM | 123 | CA | GLN | A | 19 | −22.843 | −62.418 | −5.943 | 1.00 | 19.54 | A | C |
| ATOM | 124 | CB | GLN | A | 19 | −23.100 | −63.901 | −6.252 | 1.00 | 21.61 | A | C |
| ATOM | 125 | CG | GLN | A | 19 | −24.527 | −64.361 | −5.945 | 1.00 | 22.69 | A | C |
| ATOM | 126 | CD | GLN | A | 19 | −24.762 | −65.830 | −6.230 | 1.00 | 22.70 | A | C |
| ATOM | 127 | OE1 | GLN | A | 19 | −23.902 | −66.679 | −6.031 | 1.00 | 22.41 | A | O |
| ATOM | 128 | NE2 | GLN | A | 19 | −25.944 | −66.128 | −6.686 | 1.00 | 23.42 | A | N |
| ATOM | 129 | C | GLN | A | 19 | −23.281 | −62.147 | −4.541 | 1.00 | 19.39 | A | C |
| ATOM | 130 | O | GLN | A | 19 | −22.680 | −62.642 | −3.603 | 1.00 | 19.37 | A | O |
| ATOM | 131 | N | SER | A | 20 | −24.364 | −61.405 | −4.416 | 1.00 | 20.36 | A | N |
| ATOM | 132 | CA | SER | A | 20 | −24.805 | −60.864 | −3.142 | 1.00 | 21.93 | A | C |
| ATOM | 133 | CB | SER | A | 20 | −24.242 | −59.446 | −2.917 | 1.00 | 20.94 | A | C |
| ATOM | 134 | OG | SER | A | 20 | −24.848 | −58.804 | −1.798 | 1.00 | 20.47 | A | O |
| ATOM | 135 | C | SER | A | 20 | −26.316 | −60.793 | −3.153 | 1.00 | 24.09 | A | C |
| ATOM | 136 | O | SER | A | 20 | −26.900 | −59.913 | −3.817 | 1.00 | 24.70 | A | O |
| ATOM | 137 | N | GLY | A | 21 | −26.941 | −61.719 | −2.431 | 1.00 | 25.06 | A | N |
| ATOM | 138 | CA | GLY | A | 21 | −28.389 | −61.717 | −2.286 | 1.00 | 25.86 | A | C |
| ATOM | 139 | C | GLY | A | 21 | −29.009 | −62.180 | −3.585 | 1.00 | 26.74 | A | C |
| ATOM | 140 | O | GLY | A | 21 | −28.612 | −63.233 | −4.137 | 1.00 | 26.33 | A | O |
| ATOM | 141 | N | SER | A | 22 | −29.973 | −61.376 | −4.055 | 1.00 | 26.86 | A | N |
| ATOM | 142 | CA | SER | A | 22 | −30.620 | −61.522 | −5.374 | 1.00 | 26.16 | A | C |
| ATOM | 143 | CB | SER | A | 22 | −31.916 | −60.704 | −5.410 | 1.00 | 25.69 | A | C |
| ATOM | 144 | OG | SER | A | 22 | −32.699 | −60.973 | −4.264 | 1.00 | 25.00 | A | O |
| ATOM | 145 | C | SER | A | 22 | −29.765 | −61.040 | −6.537 | 1.00 | 25.41 | A | C |
| ATOM | 146 | O | SER | A | 22 | −30.092 | −61.342 | −7.678 | 1.00 | 28.42 | A | O |
| ATOM | 147 | N | THR | A | 23 | −28.692 | −60.295 | −6.259 | 1.00 | 24.03 | A | N |
| ATOM | 148 | CA | THR | A | 23 | −27.894 | −59.630 | −7.305 | 1.00 | 23.15 | A | C |
| ATOM | 149 | CB | THR | A | 23 | −27.851 | −58.107 | −7.116 | 1.00 | 22.84 | A | C |
| ATOM | 150 | OG1 | THR | A | 23 | −28.241 | −57.818 | −5.783 | 1.00 | 23.29 | A | O |
| ATOM | 151 | CG2 | THR | A | 23 | −28.727 | −57.393 | −8.116 | 1.00 | 23.29 | A | C |
| ATOM | 152 | C | THR | A | 23 | −26.442 | −59.995 | −7.328 | 1.00 | 21.31 | A | C |
| ATOM | 153 | O | THR | A | 23 | −26.004 | −60.983 | −6.747 | 1.00 | 20.69 | A | O |
| ATOM | 154 | N | TYR | A | 24 | −25.721 | −59.153 | −8.058 | 1.00 | 20.03 | A | N |
| ATOM | 155 | CA | TYR | A | 24 | −24.288 | −59.137 | −8.090 | 1.00 | 19.44 | A | C |
| ATOM | 156 | CB | TYR | A | 24 | −23.808 | −59.724 | −9.415 | 1.00 | 20.32 | A | C |
| ATOM | 157 | CG | TYR | A | 24 | −24.253 | −61.153 | −9.647 | 1.00 | 20.98 | A | C |
| ATOM | 158 | CD1 | TYR | A | 24 | −25.577 | −61.463 | −10.012 | 1.00 | 22.25 | A | C |
| ATOM | 159 | CE1 | TYR | A | 24 | −25.973 | −62.786 | −10.223 | 1.00 | 22.82 | A | C |
| ATOM | 160 | CZ | TYR | A | 24 | −25.027 | −63.803 | −10.075 | 1.00 | 22.98 | A | C |
| ATOM | 161 | OH | TYR | A | 24 | −25.333 | −65.114 | −10.258 | 1.00 | 22.47 | A | O |
| ATOM | 162 | CE2 | TYR | A | 24 | −23.725 | −63.508 | −9.714 | 1.00 | 22.86 | A | C |
| ATOM | 163 | CD2 | TYR | A | 24 | −23.353 | −62.193 | −9.502 | 1.00 | 21.74 | A | C |
| ATOM | 164 | C | TYR | A | 24 | −23.790 | −57.707 | −7.944 | 1.00 | 18.03 | A | C |
| ATOM | 165 | O | TYR | A | 24 | −24.420 | −56.776 | −8.398 | 1.00 | 18.83 | A | O |
| ATOM | 166 | N | GLN | A | 25 | −22.646 | −57.554 | −7.304 | 1.00 | 16.91 | A | N |
| ATOM | 167 | CA | GLN | A | 25 | −22.035 | −56.282 | −7.074 | 1.00 | 16.05 | A | C |
| ATOM | 168 | CB | GLN | A | 25 | −21.610 | −56.204 | −5.617 | 1.00 | 17.12 | A | C |
| ATOM | 169 | CG | GLN | A | 25 | −22.750 | −56.234 | −4.604 | 1.00 | 18.02 | A | C |
| ATOM | 170 | CD | GLN | A | 25 | −22.238 | −56.131 | −3.157 | 1.00 | 18.84 | A | C |
| ATOM | 171 | OE1 | GLN | A | 25 | −21.652 | −57.073 | −2.611 | 1.00 | 18.68 | A | O |
| ATOM | 172 | NE2 | GLN | A | 25 | −22.436 | −54.961 | −2.547 | 1.00 | 19.37 | A | N |
| ATOM | 173 | C | GLN | A | 25 | −20.804 | −56.174 | −7.940 | 1.00 | 14.92 | A | C |
| ATOM | 174 | O | GLN | A | 25 | −20.054 | −57.139 | −8.082 | 1.00 | 13.71 | A | O |
| ATOM | 175 | N | LEU | A | 26 | −20.586 | −54.991 | −8.507 | 1.00 | 14.84 | A | N |
| ATOM | 176 | CA | LEU | A | 26 | −19.323 | −54.675 | −9.200 | 1.00 | 14.71 | A | C |
| ATOM | 177 | CB | LEU | A | 26 | −19.445 | −53.347 | −9.978 | 1.00 | 14.61 | A | C |
| ATOM | 178 | CG | LEU | A | 26 | −19.423 | −53.351 | −11.519 | 1.00 | 14.51 | A | C |
| ATOM | 179 | CD1 | LEU | A | 26 | −19.059 | −51.976 | −12.079 | 1.00 | 14.06 | A | C |
| ATOM | 180 | CD2 | LEU | A | 26 | −18.478 | −54.406 | −12.088 | 1.00 | 14.65 | A | C |
| ATOM | 181 | C | LEU | A | 26 | −18.128 | −54.605 | −8.242 | 1.00 | 14.74 | A | C |
| ATOM | 182 | O | LEU | A | 26 | −17.595 | −53.535 | −8.002 | 1.00 | 15.14 | A | O |
| ATOM | 183 | N | LYS | A | 27 | −17.714 | −55.748 | −7.703 | 1.00 | 15.19 | A | N |
| ATOM | 184 | CA | LYS | A | 27 | −16.706 | −55.808 | −6.643 | 1.00 | 15.66 | A | C |
| ATOM | 185 | CB | LYS | A | 27 | −17.364 | −55.826 | −5.267 | 1.00 | 17.97 | A | C |
| ATOM | 186 | CG | LYS | A | 27 | −16.461 | −55.674 | −4.013 | 1.00 | 20.16 | A | C |
| ATOM | 187 | CD | LYS | A | 27 | −17.235 | −56.135 | −2.756 | 1.00 | 22.07 | A | C |
| ATOM | 188 | CE | LYS | A | 27 | −16.523 | −55.982 | −1.402 | 1.00 | 23.86 | A | C |
| ATOM | 189 | NZ | LYS | A | 27 | −16.851 | −54.674 | −0.719 | 1.00 | 24.48 | A | N |
| ATOM | 190 | C | LYS | A | 27 | −15.913 | −57.057 | −6.827 | 1.00 | 14.55 | A | C |
| ATOM | 191 | O | LYS | A | 27 | −16.489 | −58.120 | −6.996 | 1.00 | 14.47 | A | O |
| ATOM | 192 | N | ASP | A | 28 | −14.587 | −56.917 | −6.825 | 1.00 | 13.97 | A | N |
| ATOM | 193 | CA | ASP | A | 28 | −13.659 | −58.031 | −7.050 | 1.00 | 13.42 | A | C |
| ATOM | 194 | CB | ASP | A | 28 | −12.763 | −57.725 | −8.266 | 1.00 | 13.08 | A | C |
| ATOM | 195 | CG | ASP | A | 28 | −11.674 | −58.771 | −8.521 | 1.00 | 13.48 | A | C |
| ATOM | 196 | OD1 | ASP | A | 28 | −11.473 | −59.690 | −7.689 | 1.00 | 14.37 | A | O |
| ATOM | 197 | OD2 | ASP | A | 28 | −11.005 | −58.691 | −9.587 | 1.00 | 12.98 | A | O |
| ATOM | 198 | C | ASP | A | 28 | −12.893 | −58.207 | −5.739 | 1.00 | 13.01 | A | C |
| ATOM | 199 | O | ASP | A | 28 | −12.178 | −57.368 | −5.343 | 1.00 | 12.32 | A | O |
| ATOM | 200 | N | THR | A | 29 | −13.117 | −59.315 | −5.068 | 1.00 | 13.89 | A | N |
| ATOM | 201 | CA | THR | A | 29 | −12.519 | −59.651 | −3.769 | 1.00 | 14.71 | A | C |
| ATOM | 202 | CB | THR | A | 29 | −13.399 | −60.741 | −3.052 | 1.00 | 15.35 | A | C |
| ATOM | 203 | OG1 | THR | A | 29 | −14.777 | −60.336 | −3.027 | 1.00 | 15.18 | A | O |
| ATOM | 204 | CG2 | THR | A | 29 | −12.900 | −61.030 | −1.614 | 1.00 | 16.17 | A | C |
| ATOM | 205 | C | THR | A | 29 | −11.158 | −60.301 | −3.937 | 1.00 | 14.50 | A | C |
| ATOM | 206 | O | THR | A | 29 | −10.416 | −60.477 | −2.966 | 1.00 | 14.03 | A | O |
| ATOM | 207 | N | THR | A | 30 | −10.858 | −60.707 | −5.166 | 1.00 | 14.01 | A | N |
| ATOM | 208 | CA | THR | A | 30 | −9.723 | −61.573 | −5.413 | 1.00 | 13.87 | A | C |
| ATOM | 209 | CB | THR | A | 30 | −9.932 | −62.391 | −6.680 | 1.00 | 13.93 | A | C |
| ATOM | 210 | OG1 | THR | A | 30 | −9.673 | −61.570 | −7.827 | 1.00 | 14.25 | A | O |
| ATOM | 211 | CG2 | THR | A | 30 | −11.361 | −62.916 | −6.702 | 1.00 | 13.81 | A | C |
| ATOM | 212 | C | THR | A | 30 | −8.429 | −60.802 | −5.518 | 1.00 | 13.58 | A | C |
| ATOM | 213 | O | THR | A | 30 | −7.359 | −61.401 | −5.502 | 1.00 | 12.36 | A | O |
| ATOM | 214 | N | ARG | A | 31 | −8.521 | −59.476 | −5.615 | 1.00 | 14.16 | A | N |
| ATOM | 215 | CA | ARG | A | 31 | −7.297 | −58.698 | −5.580 | 1.00 | 15.08 | A | C |
| ATOM | 216 | CB | ARG | A | 31 | −6.770 | −58.358 | −6.990 | 1.00 | 15.55 | A | C |
| ATOM | 217 | CG | ARG | A | 31 | −7.598 | −57.511 | −7.921 | 1.00 | 16.02 | A | C |
| ATOM | 218 | CD | ARG | A | 31 | −8.195 | −58.329 | −9.061 | 1.00 | 16.47 | A | C |
| ATOM | 219 | NE | ARG | A | 31 | −7.390 | −58.608 | −10.270 | 1.00 | 15.99 | A | N |
| ATOM | 220 | CZ | ARG | A | 31 | −7.738 | −59.539 | −11.181 | 1.00 | 15.52 | A | C |
| ATOM | 221 | NH1 | ARG | A | 31 | −8.823 | −60.305 | −11.035 | 1.00 | 15.13 | A | N |
| ATOM | 222 | NH2 | ARG | A | 31 | −6.991 | −59.741 | −12.238 | 1.00 | 15.37 | A | N |
| ATOM | 223 | C | ARG | A | 31 | −7.235 | −57.525 | −4.589 | 1.00 | 14.78 | A | C |
| ATOM | 224 | O | ARG | A | 31 | −8.080 | −56.638 | −4.583 | 1.00 | 14.51 | A | O |
| ATOM | 225 | N | GLY | A | 32 | −6.182 | −57.584 | −3.753 | 1.00 | 14.67 | A | N |
| ATOM | 226 | CA | GLY | A | 32 | −6.087 | −56.875 | −2.471 | 1.00 | 14.18 | A | C |
| ATOM | 227 | C | GLY | A | 32 | −7.325 | −56.959 | −1.608 | 1.00 | 13.91 | A | C |
| ATOM | 228 | O | GLY | A | 32 | −8.032 | −57.952 | −1.604 | 1.00 | 13.27 | A | O |
| ATOM | 229 | N | GLN | A | 33 | −7.608 | −55.897 | −0.875 | 1.00 | 14.47 | A | N |
| ATOM | 230 | CA | GLN | A | 33 | −8.876 | −55.840 | −0.160 | 1.00 | 15.13 | A | C |
| ATOM | 231 | CB | GLN | A | 33 | −8.834 | −54.815 | 0.957 | 1.00 | 16.42 | A | C |
| ATOM | 232 | CG | GLN | A | 33 | −7.964 | −55.204 | 2.146 | 1.00 | 17.07 | A | C |
| ATOM | 233 | CD | GLN | A | 33 | −7.000 | −54.094 | 2.468 | 1.00 | 18.22 | A | C |
| ATOM | 234 | OE1 | GLN | A | 33 | −5.796 | −54.344 | 2.529 | 1.00 | 18.93 | A | O |
| ATOM | 235 | NE2 | GLN | A | 33 | −7.516 | −52.821 | 2.619 | 1.00 | 18.12 | A | N |
| ATOM | 236 | C | GLN | A | 33 | −10.024 | −55.539 | −1.099 | 1.00 | 14.63 | A | C |
| ATOM | 237 | O | GLN | A | 33 | −11.164 | −55.372 | −0.671 | 1.00 | 14.46 | A | O |
| ATOM | 238 | N | GLY | A | 34 | −9.727 | −55.477 | −2.386 | 1.00 | 14.20 | A | N |
| ATOM | 239 | CA | GLY | A | 34 | −10.766 | −55.551 | −3.369 | 1.00 | 13.93 | A | C |
| ATOM | 240 | C | GLY | A | 34 | −10.802 | −54.379 | −4.303 | 1.00 | 13.69 | A | C |
| ATOM | 241 | O | GLY | A | 34 | −10.151 | −53.360 | −4.073 | 1.00 | 14.83 | A | O |
| ATOM | 242 | N | ILE | A | 35 | −11.563 | −54.533 | −5.371 | 1.00 | 12.80 | A | N |
| ATOM | 243 | CA | ILE | A | 35 | −11.706 | −53.495 | −6.353 | 1.00 | 12.48 | A | C |
| ATOM | 244 | CB | ILE | A | 35 | −11.136 | −53.951 | −7.695 | 1.00 | 12.26 | A | C |
| ATOM | 245 | CG1 | ILE | A | 35 | −9.639 | −54.116 | −7.554 | 1.00 | 12.72 | A | C |
| ATOM | 246 | CD1 | ILE | A | 35 | −8.906 | −54.294 | −8.876 | 1.00 | 13.19 | A | C |
| ATOM | 247 | CG2 | ILE | A | 35 | −11.468 | −52.976 | −8.798 | 1.00 | 12.20 | A | C |
| ATOM | 248 | C | ILE | A | 35 | −13.193 | −53.286 | −6.417 | 1.00 | 12.31 | A | C |
| ATOM | 249 | O | ILE | A | 35 | −13.938 | −54.242 | −6.575 | 1.00 | 12.03 | A | O |
| ATOM | 250 | N | VAL | A | 36 | −13.625 | −52.043 | −6.257 | 1.00 | 12.28 | A | N |
| ATOM | 251 | CA | VAL | A | 36 | −15.051 | −51.755 | −6.160 | 1.00 | 12.25 | A | C |
| ATOM | 252 | CB | VAL | A | 36 | −15.459 | −51.351 | −4.737 | 1.00 | 12.50 | A | C |
| ATOM | 253 | CG1 | VAL | A | 36 | −16.968 | −51.376 | −4.631 | 1.00 | 12.79 | A | C |
| ATOM | 254 | CG2 | VAL | A | 36 | −14.829 | −52.271 | −3.691 | 1.00 | 12.49 | A | C |
| ATOM | 255 | C | VAL | A | 36 | −15.378 | −50.600 | −7.042 | 1.00 | 11.79 | A | C |
| ATOM | 256 | O | VAL | A | 36 | −14.656 | −49.627 | −7.068 | 1.00 | 11.66 | A | O |
| ATOM | 257 | N | THR | A | 37 | −16.480 | −50.693 | −7.752 | 1.00 | 11.73 | A | N |
| ATOM | 258 | CA | THR | A | 37 | −16.821 | −49.656 | −8.699 | 1.00 | 11.95 | A | C |
| ATOM | 259 | CB | THR | A | 37 | −16.651 | −50.214 | −10.125 | 1.00 | 12.12 | A | C |
| ATOM | 260 | OG1 | THR | A | 37 | −15.306 | −50.678 | −10.323 | 1.00 | 11.60 | A | O |
| ATOM | 261 | CG2 | THR | A | 37 | −16.978 | −49.158 | −11.163 | 1.00 | 12.41 | A | C |
| ATOM | 262 | C | THR | A | 37 | −18.242 | −49.064 | −8.450 | 1.00 | 12.02 | A | C |
| ATOM | 263 | O | THR | A | 37 | −19.220 | −49.806 | −8.348 | 1.00 | 11.27 | A | O |
| ATOM | 264 | N | TYR | A | 38 | −18.310 | −47.727 | −8.348 | 1.00 | 12.30 | A | N |
| ATOM | 265 | CA | TYR | A | 38 | −19.531 | −46.984 | −8.027 | 1.00 | 12.80 | A | C |
| ATOM | 266 | CB | TYR | A | 38 | −19.301 | −46.102 | −6.793 | 1.00 | 13.01 | A | C |
| ATOM | 267 | CG | TYR | A | 38 | −18.714 | −46.788 | −5.592 | 1.00 | 13.05 | A | C |
| ATOM | 268 | CD1 | TYR | A | 38 | −17.349 | −47.059 | −5.500 | 1.00 | 12.97 | A | C |
| ATOM | 269 | CE1 | TYR | A | 38 | −16.826 | −47.706 | −4.387 | 1.00 | 12.85 | A | C |
| ATOM | 270 | CZ | TYR | A | 38 | −17.664 | −48.041 | −3.345 | 1.00 | 12.79 | A | C |
| ATOM | 271 | OH | TYR | A | 38 | −17.222 | −48.655 | −2.229 | 1.00 | 13.25 | A | O |
| ATOM | 272 | CE2 | TYR | A | 38 | −18.991 | −47.767 | −3.402 | 1.00 | 13.05 | A | C |
| ATOM | 273 | CD2 | TYR | A | 38 | −19.519 | −47.145 | −4.523 | 1.00 | 13.15 | A | C |
| ATOM | 274 | C | TYR | A | 38 | −19.921 | −46.001 | −9.123 | 1.00 | 13.22 | A | C |
| ATOM | 275 | O | TYR | A | 38 | −19.061 | −45.563 | −9.886 | 1.00 | 13.13 | A | O |
| ATOM | 276 | N | SER | A | 39 | −21.196 | −45.588 | −9.132 | 1.00 | 13.71 | A | N |
| ATOM | 277 | CA | SER | A | 39 | −21.668 | −44.417 | −9.919 | 1.00 | 14.12 | A | C |
| ATOM | 278 | CB | SER | A | 39 | −22.854 | −44.784 | −10.823 | 1.00 | 13.74 | A | C |
| ATOM | 279 | OG | SER | A | 39 | −23.634 | −43.628 | −11.165 | 1.00 | 13.56 | A | O |
| ATOM | 280 | C | SER | A | 39 | −22.052 | −43.226 | −9.009 | 1.00 | 14.90 | A | C |
| ATOM | 281 | O | SER | A | 39 | −22.651 | −43.409 | −7.942 | 1.00 | 15.79 | A | O |
| ATOM | 282 | N | ALA | A | 40 | −21.697 | −42.011 | −9.422 | 1.00 | 15.34 | A | N |
| ATOM | 283 | CA | ALA | A | 40 | −22.070 | −40.810 | −8.672 | 1.00 | 15.65 | A | C |
| ATOM | 284 | CB | ALA | A | 40 | −21.026 | −39.738 | −8.857 | 1.00 | 15.26 | A | C |
| ATOM | 285 | C | ALA | A | 40 | −23.444 | −40.255 | −9.080 | 1.00 | 16.51 | A | C |
| ATOM | 286 | O | ALA | A | 40 | −23.871 | −39.252 | −8.542 | 1.00 | 16.26 | A | O |
| ATOM | 287 | N | GLY | A | 41 | −24.125 | −40.884 | −10.037 | 1.00 | 17.11 | A | N |
| ATOM | 288 | CA | GLY | A | 41 | −25.400 | −40.386 | −10.509 | 1.00 | 17.44 | A | C |
| ATOM | 289 | C | GLY | A | 41 | −25.335 | −38.944 | −10.945 | 1.00 | 18.31 | A | C |
| ATOM | 290 | O | GLY | A | 41 | −26.259 | −38.156 | −10.687 | 1.00 | 18.80 | A | O |
| ATOM | 291 | N | ASN | A | 42 | −24.230 | −38.586 | −11.583 | 1.00 | 18.86 | A | N |
| ATOM | 292 | CA | ASN | A | 42 | −24.007 | −37.213 | −12.060 | 1.00 | 19.31 | A | C |
| ATOM | 293 | CB | ASN | A | 42 | −24.917 | −36.897 | −13.253 | 1.00 | 18.59 | A | C |
| ATOM | 294 | CG | ASN | A | 42 | −24.664 | −37.831 | −14.428 | 1.00 | 18.02 | A | C |
| ATOM | 295 | OD1 | ASN | A | 42 | −25.567 | −38.474 | −14.893 | 1.00 | 18.09 | A | O |
| ATOM | 296 | ND2 | ASN | A | 42 | −23.419 | −37.928 | −14.879 | 1.00 | 17.76 | A | N |
| ATOM | 297 | C | ASN | A | 42 | −24.075 | −36.119 | −11.010 | 1.00 | 20.65 | A | C |
| ATOM | 298 | O | ASN | A | 42 | −24.293 | −34.976 | −11.332 | 1.00 | 20.22 | A | O |
| ATOM | 299 | N | ARG | A | 43 | −23.827 | −36.470 | −9.761 | 1.00 | 23.59 | A | N |
| ATOM | 300 | CA | ARG | A | 43 | −23.646 | −35.496 | −8.707 | 1.00 | 26.15 | A | C |
| ATOM | 301 | CB | ARG | A | 43 | −24.688 | −35.719 | −7.618 | 1.00 | 30.73 | A | C |
| ATOM | 302 | CG | ARG | A | 43 | −26.103 | −35.379 | −8.056 | 1.00 | 35.36 | A | C |
| ATOM | 303 | CD | ARG | A | 43 | −27.117 | −36.068 | −7.163 | 1.00 | 42.30 | A | C |
| ATOM | 304 | NE | ARG | A | 43 | −28.483 | −35.870 | −7.661 | 1.00 | 51.16 | A | N |
| ATOM | 305 | CZ | ARG | A | 43 | −29.263 | −36.813 | −8.216 | 1.00 | 54.64 | A | C |
| ATOM | 306 | NH1 | ARG | A | 43 | −28.842 | −38.082 | −8.366 | 1.00 | 55.48 | A | N |
| ATOM | 307 | NH2 | ARG | A | 43 | −30.493 | −36.474 | −8.620 | 1.00 | 51.20 | A | N |
| ATOM | 308 | C | ARG | A | 43 | −22.227 | −35.710 | −8.203 | 1.00 | 25.25 | A | C |
| ATOM | 309 | O | ARG | A | 43 | −21.465 | −36.443 | −8.850 | 1.00 | 25.16 | A | O |
| ATOM | 310 | N | SER | A | 44 | −21.856 | −35.091 | −7.080 | 1.00 | 23.65 | A | N |
| ATOM | 311 | CA | SER | A | 44 | −20.469 | −35.117 | −6.638 | 1.00 | 23.20 | A | C |
| ATOM | 312 | CB | SER | A | 44 | −19.913 | −33.725 | −6.728 | 1.00 | 23.30 | A | C |
| ATOM | 313 | OG | SER | A | 44 | −20.575 | −32.930 | −5.806 | 1.00 | 24.80 | A | O |
| ATOM | 314 | C | SER | A | 44 | −20.254 | −35.685 | −5.235 | 1.00 | 22.60 | A | C |
| ATOM | 315 | O | SER | A | 44 | −19.168 | −35.591 | −4.637 | 1.00 | 23.35 | A | O |
| ATOM | 316 | N | SER | A | 45 | −21.291 | −36.310 | −4.728 | 1.00 | 21.47 | A | N |
| ATOM | 317 | CA | SER | A | 45 | −21.198 | −37.091 | −3.516 | 1.00 | 20.72 | A | C |
| ATOM | 318 | CB | SER | A | 45 | −22.604 | −37.567 | −3.142 | 1.00 | 20.80 | A | C |
| ATOM | 319 | OG | SER | A | 45 | −22.968 | −37.078 | −1.894 | 1.00 | 21.92 | A | O |
| ATOM | 320 | C | SER | A | 45 | −20.325 | −38.324 | −3.784 | 1.00 | 19.65 | A | C |
| ATOM | 321 | O | SER | A | 45 | −20.563 | −39.037 | −4.775 | 1.00 | 19.28 | A | O |
| ATOM | 322 | N | LEU | A | 46 | −19.357 | −38.602 | −2.901 | 1.00 | 17.76 | A | N |
| ATOM | 323 | CA | LEU | A | 46 | −18.494 | −39.787 | −3.055 | 1.00 | 16.26 | A | C |
| ATOM | 324 | CB | LEU | A | 46 | −17.059 | −39.358 | −3.308 | 1.00 | 15.57 | A | C |
| ATOM | 325 | CG | LEU | A | 46 | −16.799 | −38.486 | −4.528 | 1.00 | 15.28 | A | C |
| ATOM | 326 | CD1 | LEU | A | 46 | −15.336 | −38.131 | −4.537 | 1.00 | 15.11 | A | C |
| ATOM | 327 | CD2 | LEU | A | 46 | −17.173 | −39.184 | −5.834 | 1.00 | 15.38 | A | C |
| ATOM | 328 | C | LEU | A | 46 | −18.528 | −40.707 | −1.854 | 1.00 | 15.97 | A | C |
| ATOM | 329 | O | LEU | A | 46 | −18.512 | −40.255 | −0.740 | 1.00 | 15.97 | A | O |
| ATOM | 330 | N | PRO | A | 47 | −18.450 | −42.021 | −2.068 | 1.00 | 16.23 | A | N |
| ATOM | 331 | CA | PRO | A | 47 | −18.098 | −42.713 | −3.302 | 1.00 | 16.33 | A | C |
| ATOM | 332 | CB | PRO | A | 47 | −17.593 | −44.069 | −2.806 | 1.00 | 16.02 | A | C |
| ATOM | 333 | CG | PRO | A | 47 | −17.843 | −44.110 | −1.340 | 1.00 | 15.83 | A | C |
| ATOM | 334 | CD | PRO | A | 47 | −18.710 | −42.968 | −0.980 | 1.00 | 15.87 | A | C |
| ATOM | 335 | C | PRO | A | 47 | −19.271 | −42.898 | −4.262 | 1.00 | 16.88 | A | C |
| ATOM | 336 | O | PRO | A | 47 | −19.062 | −43.188 | −5.448 | 1.00 | 16.98 | A | O |
| ATOM | 337 | N | GLY | A | 48 | −20.496 | −42.741 | −3.752 | 1.00 | 16.89 | A | N |
| ATOM | 338 | CA | GLY | A | 48 | −21.680 | −42.866 | −4.577 | 1.00 | 15.71 | A | C |
| ATOM | 339 | C | GLY | A | 48 | −22.281 | −44.214 | −4.323 | 1.00 | 14.63 | A | C |
| ATOM | 340 | O | GLY | A | 48 | −21.984 | −44.833 | −3.317 | 1.00 | 13.70 | A | O |
| ATOM | 341 | N | THR | A | 49 | −23.111 | −44.644 | −5.262 | 1.00 | 14.42 | A | N |
| ATOM | 342 | CA | THR | A | 49 | −23.844 | −45.889 | −5.184 | 1.00 | 15.14 | A | C |
| ATOM | 343 | CB | THR | A | 49 | −25.186 | −45.825 | −5.997 | 1.00 | 15.13 | A | C |
| ATOM | 344 | OG1 | THR | A | 49 | −25.981 | −44.687 | −5.619 | 1.00 | 14.36 | A | O |
| ATOM | 345 | CG2 | THR | A | 49 | −25.983 | −47.129 | −5.846 | 1.00 | 14.54 | A | C |
| ATOM | 346 | C | THR | A | 49 | −23.005 | −46.995 | −5.821 | 1.00 | 15.82 | A | C |
| ATOM | 347 | O | THR | A | 49 | −22.654 | −46.911 | −7.008 | 1.00 | 15.76 | A | O |
| ATOM | 348 | N | LEU | A | 50 | −22.696 | −48.036 | −5.055 | 1.00 | 16.29 | A | N |
| ATOM | 349 | CA | LEU | A | 50 | −21.971 | −49.177 | −5.612 | 1.00 | 16.67 | A | C |
| ATOM | 350 | CB | LEU | A | 50 | −21.402 | −50.030 | −4.494 | 1.00 | 16.62 | A | C |
| ATOM | 351 | CG | LEU | A | 50 | −21.206 | −51.532 | −4.619 | 1.00 | 17.18 | A | C |
| ATOM | 352 | CD1 | LEU | A | 50 | −20.505 | −51.964 | −5.905 | 1.00 | 17.48 | A | C |
| ATOM | 353 | CD2 | LEU | A | 50 | −20.430 | −51.992 | −3.379 | 1.00 | 17.09 | A | C |
| ATOM | 354 | C | LEU | A | 50 | −22.908 | −49.955 | −6.527 | 1.00 | 16.87 | A | C |
| ATOM | 355 | O | LEU | A | 50 | −24.039 | −50.225 | −6.155 | 1.00 | 18.03 | A | O |
| ATOM | 356 | N | LEU | A | 51 | −22.438 | −50.289 | −7.724 | 1.00 | 16.66 | A | N |
| ATOM | 357 | CA | LEU | A | 51 | −23.304 | −50.807 | −8.767 | 1.00 | 16.90 | A | C |
| ATOM | 358 | CB | LEU | A | 51 | −22.702 | −50.580 | −10.165 | 1.00 | 17.78 | A | C |
| ATOM | 359 | CG | LEU | A | 51 | −22.995 | −49.231 | −10.854 | 1.00 | 18.55 | A | C |
| ATOM | 360 | CD1 | LEU | A | 51 | −21.780 | −48.321 | −10.737 | 1.00 | 18.87 | A | C |
| ATOM | 361 | CD2 | LEU | A | 51 | −23.352 | −49.435 | −12.335 | 1.00 | 19.37 | A | C |
| ATOM | 362 | C | LEU | A | 51 | −23.664 | −52.270 | −8.629 | 1.00 | 16.37 | A | C |
| ATOM | 363 | O | LEU | A | 51 | −22.888 | −53.090 | −8.130 | 1.00 | 16.35 | A | O |
| ATOM | 364 | N | THR | A | 52 | −24.843 | −52.591 | −9.151 | 1.00 | 16.12 | A | N |
| ATOM | 365 | CA | THR | A | 52 | −25.398 | −53.941 | −9.097 | 1.00 | 15.93 | A | C |
| ATOM | 366 | CB | THR | A | 52 | −26.453 | −54.014 | −7.998 | 1.00 | 15.10 | A | C |
| ATOM | 367 | OG1 | THR | A | 52 | −27.129 | −52.773 | −7.961 | 1.00 | 14.51 | A | O |
| ATOM | 368 | CG2 | THR | A | 52 | −25.826 | −54.189 | −6.670 | 1.00 | 15.28 | A | C |
| ATOM | 369 | C | THR | A | 52 | −26.087 | −54.312 | −10.398 | 1.00 | 16.34 | A | C |
| ATOM | 370 | O | THR | A | 52 | −26.640 | −53.474 | −11.070 | 1.00 | 16.38 | A | O |
| ATOM | 371 | N | SER | A | 53 | −26.044 | −55.575 | −10.764 | 1.00 | 17.42 | A | N |
| ATOM | 372 | CA | SER | A | 53 | −26.900 | −56.061 | −11.829 | 1.00 | 18.54 | A | C |
| ATOM | 373 | CB | SER | A | 53 | −26.179 | −56.074 | −13.170 | 1.00 | 19.26 | A | C |
| ATOM | 374 | OG | SER | A | 53 | −26.656 | −57.168 | −13.945 | 1.00 | 19.86 | A | O |
| ATOM | 375 | C | SER | A | 53 | −27.334 | −57.468 | −11.510 | 1.00 | 18.55 | A | C |
| ATOM | 376 | O | SER | A | 53 | −26.473 | −58.367 | −11.355 | 1.00 | 17.72 | A | O |
| ATOM | 377 | N | SER | A | 54 | −28.651 | −57.665 | −11.445 | 1.00 | 18.20 | A | N |
| ATOM | 378 | CA | SER | A | 54 | −29.183 | −58.969 | −11.039 | 1.00 | 19.04 | A | C |
| ATOM | 379 | CB | SER | A | 54 | −30.671 | −58.894 | −10.731 | 1.00 | 19.67 | A | C |
| ATOM | 380 | OG | SER | A | 54 | −31.362 | −58.586 | −11.907 | 1.00 | 21.03 | A | O |
| ATOM | 381 | C | SER | A | 54 | −28.905 | −60.070 | −12.047 | 1.00 | 17.88 | A | C |
| ATOM | 382 | O | SER | A | 54 | −28.803 | −61.243 | −11.644 | 1.00 | 18.22 | A | O |
| ATOM | 383 | N | SER | A | 55 | −28.737 | −59.695 | −13.324 | 1.00 | 16.80 | A | N |
| ATOM | 384 | CA | SER | A | 55 | −28.274 | −60.644 | −14.363 | 1.00 | 16.20 | A | C |
| ATOM | 385 | CB | SER | A | 55 | −28.701 | −60.186 | −15.757 | 1.00 | 15.87 | A | C |
| ATOM | 386 | OG | SER | A | 55 | −28.447 | −58.827 | −15.969 | 1.00 | 15.88 | A | O |
| ATOM | 387 | C | SER | A | 55 | −26.775 | −60.983 | −14.384 | 1.00 | 15.91 | A | C |
| ATOM | 388 | O | SER | A | 55 | −26.388 | −61.948 | −15.025 | 1.00 | 15.99 | A | O |
| ATOM | 389 | N | ASN | A | 56 | −25.947 | −60.220 | −13.671 | 1.00 | 16.14 | A | N |
| ATOM | 390 | CA | ASN | A | 56 | −24.472 | −60.215 | −13.852 | 1.00 | 15.95 | A | C |
| ATOM | 391 | CB | ASN | A | 56 | −23.805 | −61.536 | −13.382 | 1.00 | 15.78 | A | C |
| ATOM | 392 | CG | ASN | A | 56 | −22.360 | −61.358 | −12.860 | 1.00 | 15.19 | A | C |
| ATOM | 393 | OD1 | ASN | A | 56 | −21.554 | −62.306 | −12.853 | 1.00 | 14.47 | A | O |
| ATOM | 394 | ND2 | ASN | A | 56 | −22.041 | −60.169 | −12.400 | 1.00 | 14.98 | A | N |
| ATOM | 395 | C | ASN | A | 56 | −24.092 | −59.885 | −15.296 | 1.00 | 16.66 | A | C |
| ATOM | 396 | O | ASN | A | 56 | −23.018 | −60.280 | −15.756 | 1.00 | 16.28 | A | O |
| ATOM | 397 | N | ILE | A | 57 | −24.984 | −59.161 | −15.989 | 1.00 | 17.56 | A | N |
| ATOM | 398 | CA | ILE | A | 57 | −24.682 | −58.525 | −17.259 | 1.00 | 18.93 | A | C |
| ATOM | 399 | CB | ILE | A | 57 | −25.650 | −58.958 | −18.366 | 1.00 | 20.60 | A | C |
| ATOM | 400 | CG1 | ILE | A | 57 | −25.606 | −60.475 | −18.578 | 1.00 | 21.60 | A | C |
| ATOM | 401 | CD1 | ILE | A | 57 | −26.715 | −60.933 | −19.510 | 1.00 | 21.99 | A | C |
| ATOM | 402 | CG2 | ILE | A | 57 | −25.300 | −58.247 | −19.673 | 1.00 | 20.37 | A | C |
| ATOM | 403 | C | ILE | A | 57 | −24.827 | −57.017 | −17.089 | 1.00 | 18.81 | A | C |
| ATOM | 404 | O | ILE | A | 57 | −25.830 | −56.572 | −16.550 | 1.00 | 18.38 | A | O |
| ATOM | 405 | N | TRP | A | 58 | −23.869 | −56.241 | −17.603 | 1.00 | 18.79 | A | N |
| ATOM | 406 | CA | TRP | A | 58 | −23.663 | −54.869 | −17.151 | 1.00 | 19.08 | A | C |
| ATOM | 407 | CB | TRP | A | 58 | −22.285 | −54.753 | −16.467 | 1.00 | 18.65 | A | C |
| ATOM | 408 | CG | TRP | A | 58 | −22.126 | −55.653 | −15.263 | 1.00 | 17.04 | A | C |
| ATOM | 409 | CD1 | TRP | A | 58 | −21.699 | −56.943 | −15.248 | 1.00 | 16.33 | A | C |
| ATOM | 410 | NE1 | TRP | A | 58 | −21.716 | −57.436 | −13.972 | 1.00 | 15.66 | A | N |
| ATOM | 411 | CE2 | TRP | A | 58 | −22.166 | −56.454 | −13.135 | 1.00 | 15.87 | A | C |
| ATOM | 412 | CD2 | TRP | A | 58 | −22.431 | −55.318 | −13.921 | 1.00 | 15.82 | A | C |
| ATOM | 413 | CE3 | TRP | A | 58 | −22.930 | −54.178 | −13.310 | 1.00 | 15.17 | A | C |
| ATOM | 414 | CZ3 | TRP | A | 58 | −23.119 | −54.190 | −11.949 | 1.00 | 15.35 | A | C |
| ATOM | 415 | CH2 | TRP | A | 58 | −22.833 | −55.320 | −11.181 | 1.00 | 15.47 | A | C |
| ATOM | 416 | CZ2 | TRP | A | 58 | −22.357 | −56.465 | −11.757 | 1.00 | 15.91 | A | C |
| ATOM | 417 | C | TRP | A | 58 | −23.743 | −53.866 | −18.273 | 1.00 | 21.25 | A | C |
| ATOM | 418 | O | TRP | A | 58 | −22.885 | −53.847 | −19.163 | 1.00 | 21.87 | A | O |
| ATOM | 419 | N | ASN | A | 59 | −24.748 | −52.994 | −18.211 | 1.00 | 23.98 | A | N |
| ATOM | 420 | CA | ASN | A | 59 | −25.040 | −52.065 | −19.306 | 1.00 | 24.38 | A | C |
| ATOM | 421 | CB | ASN | A | 59 | −26.548 | −51.940 | −19.490 | 1.00 | 26.43 | A | C |
| ATOM | 422 | CG | ASN | A | 59 | −27.209 | −51.187 | −18.334 | 1.00 | 29.93 | A | C |
| ATOM | 423 | OD1 | ASN | A | 59 | −26.529 | −50.504 | −17.551 | 1.00 | 31.64 | A | O |
| ATOM | 424 | ND2 | ASN | A | 59 | −28.536 | −51.308 | −18.213 | 1.00 | 32.05 | A | N |
| ATOM | 425 | C | ASN | A | 59 | −24.455 | −50.672 | −19.083 | 1.00 | 23.75 | A | C |
| ATOM | 426 | O | ASN | A | 59 | −24.810 | −49.744 | −19.806 | 1.00 | 27.95 | A | O |
| ATOM | 427 | N | ASP | A | 60 | −23.589 | −50.499 | −18.093 | 1.00 | 20.98 | A | N |
| ATOM | 428 | CA | ASP | A | 60 | −22.892 | −49.230 | −17.924 | 1.00 | 19.55 | A | C |
| ATOM | 429 | CB | ASP | A | 60 | −22.924 | −48.781 | −16.477 | 1.00 | 19.97 | A | C |
| ATOM | 430 | CG | ASP | A | 60 | −22.413 | −47.377 | −16.304 | 1.00 | 21.08 | A | C |
| ATOM | 431 | OD1 | ASP | A | 60 | −21.574 | −46.928 | −17.121 | 1.00 | 22.86 | A | O |
| ATOM | 432 | OD2 | ASP | A | 60 | −22.856 | −46.703 | −15.354 | 1.00 | 22.23 | A | O |
| ATOM | 433 | C | ASP | A | 60 | −21.437 | −49.304 | −18.376 | 1.00 | 18.14 | A | C |
| ATOM | 434 | O | ASP | A | 60 | −20.592 | −49.914 | −17.694 | 1.00 | 17.59 | A | O |
| ATOM | 435 | N | GLY | A | 61 | −21.133 | −48.599 | −19.466 | 1.00 | 16.48 | A | N |
| ATOM | 436 | CA | GLY | A | 61 | −19.822 | −48.709 | −20.138 | 1.00 | 15.68 | A | C |
| ATOM | 437 | C | GLY | A | 61 | −18.639 | −48.102 | −19.392 | 1.00 | 14.75 | A | C |
| ATOM | 438 | O | GLY | A | 61 | −17.577 | −48.719 | −19.298 | 1.00 | 14.18 | A | O |
| ATOM | 439 | N | ALA | A | 62 | −18.826 | −46.885 | −18.896 | 1.00 | 13.68 | A | N |
| ATOM | 440 | CA | ALA | A | 62 | −17.870 | −46.232 | −18.027 | 1.00 | 13.21 | A | C |
| ATOM | 441 | CB | ALA | A | 62 | −18.383 | −44.874 | −17.598 | 1.00 | 13.34 | A | C |
| ATOM | 442 | C | ALA | A | 62 | −17.597 | −47.074 | −16.805 | 1.00 | 12.94 | A | C |
| ATOM | 443 | O | ALA | A | 62 | −16.441 | −47.297 | −16.457 | 1.00 | 12.77 | A | O |
| ATOM | 444 | N | ALA | A | 63 | −18.646 | −47.569 | −16.161 | 1.00 | 12.70 | A | N |
| ATOM | 445 | CA | ALA | A | 63 | −18.434 | −48.493 | −15.027 | 1.00 | 12.59 | A | C |
| ATOM | 446 | CB | ALA | A | 63 | −19.759 | −48.903 | −14.401 | 1.00 | 12.74 | A | C |
| ATOM | 447 | C | ALA | A | 63 | −17.626 | −49.732 | −15.433 | 1.00 | 11.84 | A | C |
| ATOM | 448 | O | ALA | A | 63 | −16.674 | −50.096 | −14.771 | 1.00 | 11.48 | A | O |
| ATOM | 449 | N | VAL | A | 64 | −18.007 | −50.362 | −16.530 | 1.00 | 11.59 | A | N |
| ATOM | 450 | CA | VAL | A | 64 | −17.296 | −51.555 | −17.001 | 1.00 | 11.64 | A | C |
| ATOM | 451 | CB | VAL | A | 64 | −17.946 | −52.163 | −18.282 | 1.00 | 11.76 | A | C |
| ATOM | 452 | CG1 | VAL | A | 64 | −16.973 | −53.097 | −18.988 | 1.00 | 11.89 | A | C |
| ATOM | 453 | CG2 | VAL | A | 64 | −19.239 | −52.927 | −17.954 | 1.00 | 11.63 | A | C |
| ATOM | 454 | C | VAL | A | 64 | −15.811 | −51.314 | −17.274 | 1.00 | 11.25 | A | C |
| ATOM | 455 | O | VAL | A | 64 | −14.967 | −52.092 | −16.889 | 1.00 | 10.71 | A | O |
| ATOM | 456 | N | ASP | A | 65 | −15.499 | −50.244 | −17.972 | 1.00 | 11.62 | A | N |
| ATOM | 457 | CA | ASP | A | 65 | −14.103 | −49.937 | −18.299 | 1.00 | 11.62 | A | C |
| ATOM | 458 | CB | ASP | A | 65 | −13.988 | −48.750 | −19.282 | 1.00 | 11.58 | A | C |
| ATOM | 459 | CG | ASP | A | 65 | −14.272 | −49.144 | −20.735 | 1.00 | 11.39 | A | C |
| ATOM | 460 | OD1 | ASP | A | 65 | −14.119 | −50.315 | −21.053 | 1.00 | 11.29 | A | O |
| ATOM | 461 | OD2 | ASP | A | 65 | −14.647 | −48.294 | −21.563 | 1.00 | 11.28 | A | O |
| ATOM | 462 | C | ASP | A | 65 | −13.343 | −49.657 | −17.011 | 1.00 | 11.68 | A | C |
| ATOM | 463 | O | ASP | A | 65 | −12.287 | −50.237 | −16.795 | 1.00 | 12.17 | A | O |
| ATOM | 464 | N | ALA | A | 66 | −13.881 | −48.805 | −16.142 | 1.00 | 11.22 | A | N |
| ATOM | 465 | CA | ALA | A | 66 | −13.206 | −48.530 | −14.877 | 1.00 | 11.26 | A | C |
| ATOM | 466 | CB | ALA | A | 66 | −14.002 | −47.545 | −14.038 | 1.00 | 11.11 | A | C |
| ATOM | 467 | C | ALA | A | 66 | −12.942 | −49.816 | −14.076 | 1.00 | 11.77 | A | C |
| ATOM | 468 | O | ALA | A | 66 | −11.835 | −50.016 | −13.538 | 1.00 | 11.80 | A | O |
| ATOM | 469 | N | HIS | A | 67 | −13.944 | −50.690 | −13.995 | 1.00 | 11.93 | A | N |
| ATOM | 470 | CA | HIS | A | 67 | −13.758 | −51.933 | −13.275 | 1.00 | 12.20 | A | C |
| ATOM | 471 | CB | HIS | A | 67 | −15.047 | −52.745 | −13.195 | 1.00 | 12.39 | A | C |
| ATOM | 472 | CG | HIS | A | 67 | −15.079 | −53.722 | −12.053 | 1.00 | 12.80 | A | C |
| ATOM | 473 | ND1 | HIS | A | 67 | −15.102 | −53.327 | −10.736 | 1.00 | 13.19 | A | N |
| ATOM | 474 | CE1 | HIS | A | 67 | −15.143 | −54.396 | −9.955 | 1.00 | 13.25 | A | C |
| ATOM | 475 | NE2 | HIS | A | 67 | −15.176 | −55.469 | −10.719 | 1.00 | 12.96 | A | N |
| ATOM | 476 | CD2 | HIS | A | 67 | −15.137 | −55.075 | −12.033 | 1.00 | 13.09 | A | C |
| ATOM | 477 | C | HIS | A | 67 | −12.649 | −52.740 | −13.934 | 1.00 | 12.01 | A | C |
| ATOM | 478 | O | HIS | A | 67 | −11.651 | −53.041 | −13.316 | 1.00 | 12.49 | A | O |
| ATOM | 479 | N | ALA | A | 68 | −12.809 | −53.051 | −15.205 | 1.00 | 11.74 | A | N |
| ATOM | 480 | CA | ALA | A | 68 | −11.876 | −53.929 | −15.905 | 1.00 | 11.42 | A | C |
| ATOM | 481 | CB | ALA | A | 68 | −12.395 | −54.188 | −17.313 | 1.00 | 11.62 | A | C |
| ATOM | 482 | C | ALA | A | 68 | −10.449 | −53.368 | −15.986 | 1.00 | 10.67 | A | C |
| ATOM | 483 | O | ALA | A | 68 | −9.451 | −54.078 | −15.807 | 1.00 | 10.44 | A | O |
| ATOM | 484 | N | TYR | A | 69 | −10.368 | −52.089 | −16.267 | 1.00 | 10.14 | A | N |
| ATOM | 485 | CA | TYR | A | 69 | −9.083 | −51.445 | −16.372 | 1.00 | 9.92 | A | C |
| ATOM | 486 | CB | TYR | A | 69 | −9.211 | −50.127 | −17.137 | 1.00 | 9.77 | A | C |
| ATOM | 487 | CG | TYR | A | 69 | −9.568 | −50.343 | −18.608 | 1.00 | 9.70 | A | C |
| ATOM | 488 | CD1 | TYR | A | 69 | −9.263 | −51.544 | −19.258 | 1.00 | 9.60 | A | C |
| ATOM | 489 | CE1 | TYR | A | 69 | −9.588 | −51.750 | −20.584 | 1.00 | 9.57 | A | C |
| ATOM | 490 | CZ | TYR | A | 69 | −10.204 | −50.755 | −21.307 | 1.00 | 9.49 | A | C |
| ATOM | 491 | OH | TYR | A | 69 | −10.500 | −50.982 | −22.645 | 1.00 | 8.99 | A | O |
| ATOM | 492 | CE2 | TYR | A | 69 | −10.509 | −49.549 | −20.685 | 1.00 | 9.61 | A | C |
| ATOM | 493 | CD2 | TYR | A | 69 | −10.201 | −49.356 | −19.341 | 1.00 | 9.63 | A | C |
| ATOM | 494 | C | TYR | A | 69 | −8.366 | −51.278 | −15.053 | 1.00 | 9.69 | A | C |
| ATOM | 495 | O | TYR | A | 69 | −7.129 | −51.268 | −15.022 | 1.00 | 9.58 | A | O |
| ATOM | 496 | N | THR | A | 70 | −9.129 | −51.193 | −13.965 | 1.00 | 9.51 | A | N |
| ATOM | 497 | CA | THR | A | 70 | −8.532 | −51.019 | −12.645 | 1.00 | 9.27 | A | C |
| ATOM | 498 | CB | THR | A | 70 | −9.549 | −50.438 | −11.620 | 1.00 | 9.02 | A | C |
| ATOM | 499 | OG1 | THR | A | 70 | −9.925 | −49.095 | −11.972 | 1.00 | 8.65 | A | O |
| ATOM | 500 | CG2 | THR | A | 70 | −8.960 | −50.394 | −10.262 | 1.00 | 8.99 | A | C |
| ATOM | 501 | C | THR | A | 70 | −7.970 | −52.395 | −12.257 | 1.00 | 9.35 | A | C |
| ATOM | 502 | O | THR | A | 70 | −6.910 | −52.516 | −11.659 | 1.00 | 9.65 | A | O |
| ATOM | 503 | N | ALA | A | 71 | −8.655 | −53.452 | −12.651 | 1.00 | 9.39 | A | N |
| ATOM | 504 | CA | ALA | A | 71 | −8.127 | −54.785 | −12.428 | 1.00 | 9.42 | A | C |
| ATOM | 505 | CB | ALA | A | 71 | −9.190 | −55.834 | −12.721 | 1.00 | 9.15 | A | C |
| ATOM | 506 | C | ALA | A | 71 | −6.854 | −54.985 | −13.284 | 1.00 | 9.65 | A | C |
| ATOM | 507 | O | ALA | A | 71 | −5.878 | −55.594 | −12.835 | 1.00 | 9.53 | A | O |
| ATOM | 508 | N | LYS | A | 72 | −6.842 | −54.432 | −14.491 | 1.00 | 10.10 | A | N |
| ATOM | 509 | CA | LYS | A | 72 | −5.638 | −54.469 | −15.315 | 1.00 | 10.84 | A | C |
| ATOM | 510 | CB | LYS | A | 72 | −5.945 | −53.814 | −16.635 | 1.00 | 11.60 | A | C |
| ATOM | 511 | CG | LYS | A | 72 | −5.034 | −54.225 | −17.764 | 1.00 | 12.60 | A | C |
| ATOM | 512 | CD | LYS | A | 72 | −5.285 | −53.298 | −18.948 | 1.00 | 13.78 | A | C |
| ATOM | 513 | CE | LYS | A | 72 | −4.237 | −53.477 | −20.052 | 1.00 | 14.79 | A | C |
| ATOM | 514 | NZ | LYS | A | 72 | −4.525 | −54.698 | −20.852 | 1.00 | 15.32 | A | N |
| ATOM | 515 | C | LYS | A | 72 | −4.428 | −53.773 | −14.648 | 1.00 | 10.95 | A | C |
| ATOM | 516 | O | LYS | A | 72 | −3.320 | −54.296 | −14.566 | 1.00 | 10.53 | A | O |
| ATOM | 517 | N | VAL | A | 73 | −4.666 | −52.580 | −14.143 | 1.00 | 11.11 | A | N |
| ATOM | 518 | CA | VAL | A | 73 | −3.613 | −51.873 | −13.539 | 1.00 | 11.29 | A | C |
| ATOM | 519 | CB | VAL | A | 73 | −4.052 | −50.456 | −13.206 | 1.00 | 11.29 | A | C |
| ATOM | 520 | CG1 | VAL | A | 73 | −2.976 | −49.773 | −12.375 | 1.00 | 11.51 | A | C |
| ATOM | 521 | CG2 | VAL | A | 73 | −4.297 | −49.674 | −14.477 | 1.00 | 11.25 | A | C |
| ATOM | 522 | C | VAL | A | 73 | −3.179 | −52.631 | −12.286 | 1.00 | 11.79 | A | C |
| ATOM | 523 | O | VAL | A | 73 | −1.973 | −52.775 | −12.023 | 1.00 | 12.54 | A | O |
| ATOM | 524 | N | TYR | A | 74 | −4.146 | −53.094 | −11.488 | 1.00 | 11.69 | A | N |
| ATOM | 525 | CA | TYR | A | 74 | −3.803 | −53.789 | −10.253 | 1.00 | 11.38 | A | C |
| ATOM | 526 | CB | TYR | A | 74 | −5.052 | −54.356 | −9.512 | 1.00 | 10.72 | A | C |
| ATOM | 527 | CG | TYR | A | 74 | −4.642 | −55.235 | −8.341 | 1.00 | 9.87 | A | C |
| ATOM | 528 | CD1 | TYR | A | 74 | −4.236 | −56.541 | −8.538 | 1.00 | 9.52 | A | C |
| ATOM | 529 | CE1 | TYR | A | 74 | −3.798 | −57.334 | −7.492 | 1.00 | 9.27 | A | C |
| ATOM | 530 | CZ | TYR | A | 74 | −3.767 | −56.822 | −6.235 | 1.00 | 9.43 | A | C |
| ATOM | 531 | OH | TYR | A | 74 | −3.319 | −57.598 | −5.195 | 1.00 | 9.43 | A | O |
| ATOM | 532 | CE2 | TYR | A | 74 | −4.152 | −55.514 | −6.010 | 1.00 | 9.49 | A | C |
| ATOM | 533 | CD2 | TYR | A | 74 | −4.577 | −54.729 | −7.062 | 1.00 | 9.59 | A | C |
| ATOM | 534 | C | TYR | A | 74 | −2.797 | −54.923 | −10.602 | 1.00 | 12.02 | A | C |
| ATOM | 535 | O | TYR | A | 74 | −1.816 | −55.093 | −9.905 | 1.00 | 11.97 | A | O |
| ATOM | 536 | N | ASP | A | 75 | −3.062 | −55.670 | −11.683 | 1.00 | 12.59 | A | N |
| ATOM | 537 | CA | ASP | A | 75 | −2.225 | −56.802 | −12.099 | 1.00 | 13.15 | A | C |
| ATOM | 538 | CB | ASP | A | 75 | −2.863 | −57.577 | −13.258 | 1.00 | 12.65 | A | C |
| ATOM | 539 | CG | ASP | A | 75 | −4.140 | −58.272 | −12.887 | 1.00 | 12.28 | A | C |
| ATOM | 540 | OD1 | ASP | A | 75 | −4.408 | −58.613 | −11.704 | 1.00 | 11.68 | A | O |
| ATOM | 541 | OD2 | ASP | A | 75 | −4.889 | −58.488 | −13.848 | 1.00 | 12.29 | A | O |
| ATOM | 542 | C | ASP | A | 75 | −0.884 | −56.370 | −12.652 | 1.00 | 13.93 | A | C |
| ATOM | 543 | O | ASP | A | 75 | 0.029 | −57.189 | −12.744 | 1.00 | 14.31 | A | O |
| ATOM | 544 | N | TYR | A | 76 | −0.790 | −55.129 | −13.108 | 1.00 | 14.12 | A | N |
| ATOM | 545 | CA | TYR | A | 76 | 0.435 | −54.684 | −13.703 | 1.00 | 15.01 | A | C |
| ATOM | 546 | CB | TYR | A | 76 | 0.232 | −53.411 | −14.549 | 1.00 | 14.93 | A | C |
| ATOM | 547 | CG | TYR | A | 76 | 1.483 | −52.846 | −15.181 | 1.00 | 14.82 | A | C |
| ATOM | 548 | CD1 | TYR | A | 76 | 2.257 | −51.907 | −14.523 | 1.00 | 15.03 | A | C |
| ATOM | 549 | CE1 | TYR | A | 76 | 3.409 | −51.387 | −15.107 | 1.00 | 15.40 | A | C |
| ATOM | 550 | CZ | TYR | A | 76 | 3.787 | −51.815 | −16.375 | 1.00 | 15.55 | A | C |
| ATOM | 551 | OH | TYR | A | 76 | 4.929 | −51.315 | −16.990 | 1.00 | 15.74 | A | O |
| ATOM | 552 | CE2 | TYR | A | 76 | 3.022 | −52.753 | −17.036 | 1.00 | 15.06 | A | C |
| ATOM | 553 | CD2 | TYR | A | 76 | 1.880 | −53.248 | −16.444 | 1.00 | 15.02 | A | C |
| ATOM | 554 | C | TYR | A | 76 | 1.381 | −54.465 | −12.538 | 1.00 | 15.85 | A | C |
| ATOM | 555 | O | TYR | A | 76 | 2.541 | −54.932 | −12.545 | 1.00 | 16.76 | A | O |
| ATOM | 556 | N | TYR | A | 77 | 0.898 | −53.779 | −11.522 | 1.00 | 16.19 | A | N |
| ATOM | 557 | CA | TYR | A | 77 | 1.796 | −53.445 | −10.429 | 1.00 | 17.84 | A | C |
| ATOM | 558 | CB | TYR | A | 77 | 1.163 | −52.449 | −9.451 | 1.00 | 16.53 | A | C |
| ATOM | 559 | CG | TYR | A | 77 | 1.341 | −50.967 | −9.805 | 1.00 | 15.02 | A | C |
| ATOM | 560 | CD1 | TYR | A | 77 | 2.441 | −50.267 | −9.364 | 1.00 | 14.53 | A | C |
| ATOM | 561 | CE1 | TYR | A | 77 | 2.587 | −48.923 | −9.613 | 1.00 | 14.07 | A | C |
| ATOM | 562 | CZ | TYR | A | 77 | 1.622 | −48.263 | −10.311 | 1.00 | 13.99 | A | C |
| ATOM | 563 | OH | TYR | A | 77 | 1.805 | −46.916 | −10.568 | 1.00 | 14.22 | A | O |
| ATOM | 564 | CE2 | TYR | A | 77 | 0.492 | −48.927 | −10.740 | 1.00 | 13.72 | A | C |
| ATOM | 565 | CD2 | TYR | A | 77 | 0.360 | −50.266 | −10.487 | 1.00 | 14.12 | A | C |
| ATOM | 566 | C | TYR | A | 77 | 2.211 | −54.723 | −9.712 | 1.00 | 19.97 | A | C |
| ATOM | 567 | O | TYR | A | 77 | 3.325 | −54.819 | −9.174 | 1.00 | 21.51 | A | O |
| ATOM | 568 | N | LYS | A | 78 | 1.315 | −55.704 | −9.730 | 1.00 | 22.11 | A | N |
| ATOM | 569 | CA | LYS | A | 78 | 1.556 | −56.962 | −9.075 | 1.00 | 23.87 | A | C |
| ATOM | 570 | CB | LYS | A | 78 | 0.239 | −57.743 | −8.908 | 1.00 | 25.91 | A | C |
| ATOM | 571 | CG | LYS | A | 78 | 0.347 | −59.104 | −8.221 | 1.00 | 28.58 | A | C |
| ATOM | 572 | CD | LYS | A | 78 | 1.136 | −59.006 | −6.918 | 1.00 | 33.48 | A | C |
| ATOM | 573 | CE | LYS | A | 78 | 0.619 | −59.934 | −5.815 | 1.00 | 37.52 | A | C |
| ATOM | 574 | NZ | LYS | A | 78 | −0.673 | −59.444 | −5.218 | 1.00 | 39.88 | A | N |
| ATOM | 575 | C | LYS | A | 78 | 2.622 | −57.747 | −9.855 | 1.00 | 22.86 | A | C |
| ATOM | 576 | O | LYS | A | 78 | 3.600 | −58.188 | −9.247 | 1.00 | 22.16 | A | O |
| ATOM | 577 | N | ASN | A | 79 | 2.471 | −57.888 | −11.173 | 1.00 | 21.47 | A | N |
| ATOM | 578 | CA | ASN | A | 79 | 3.422 | −58.714 | −11.919 | 1.00 | 22.64 | A | C |
| ATOM | 579 | CB | ASN | A | 79 | 2.899 | −59.148 | −13.278 | 1.00 | 23.04 | A | C |
| ATOM | 580 | CG | ASN | A | 79 | 1.578 | −59.888 | −13.188 | 1.00 | 24.12 | A | C |
| ATOM | 581 | OD1 | ASN | A | 79 | 0.759 | −59.783 | −14.094 | 1.00 | 24.68 | A | O |
| ATOM | 582 | ND2 | ASN | A | 79 | 1.348 | −60.617 | −12.087 | 1.00 | 24.13 | A | N |
| ATOM | 583 | C | ASN | A | 79 | 4.769 | −58.059 | −12.103 | 1.00 | 23.13 | A | C |
| ATOM | 584 | O | ASN | A | 79 | 5.794 | −58.714 | −11.909 | 1.00 | 23.42 | A | O |
| ATOM | 585 | N | LYS | A | 80 | 4.776 | −56.780 | −12.460 | 1.00 | 23.09 | A | N |
| ATOM | 586 | CA | LYS | A | 80 | 6.024 | −56.117 | −12.778 | 1.00 | 24.20 | A | C |
| ATOM | 587 | CB | LYS | A | 80 | 5.783 | −54.871 | −13.631 | 1.00 | 27.00 | A | C |
| ATOM | 588 | CG | LYS | A | 80 | 5.314 | −55.181 | −15.037 | 1.00 | 30.36 | A | C |
| ATOM | 589 | CD | LYS | A | 80 | 6.423 | −55.858 | −15.854 | 1.00 | 33.94 | A | C |
| ATOM | 590 | CE | LYS | A | 80 | 5.916 | −57.048 | −16.688 | 1.00 | 36.49 | A | C |
| ATOM | 591 | NZ | LYS | A | 80 | 5.336 | −56.639 | −17.999 | 1.00 | 37.18 | A | N |
| ATOM | 592 | C | LYS | A | 80 | 6.841 | −55.741 | −11.554 | 1.00 | 22.72 | A | C |
| ATOM | 593 | O | LYS | A | 80 | 8.080 | −55.784 | −11.599 | 1.00 | 24.14 | A | O |
| ATOM | 594 | N | PHE | A | 81 | 6.173 | −55.352 | −10.472 | 1.00 | 20.24 | A | N |
| ATOM | 595 | CA | PHE | A | 81 | 6.876 | −54.813 | −9.318 | 1.00 | 18.15 | A | C |
| ATOM | 596 | CB | PHE | A | 81 | 6.447 | −53.380 | −9.064 | 1.00 | 18.87 | A | C |
| ATOM | 597 | CG | PHE | A | 81 | 6.461 | −52.505 | −10.289 | 1.00 | 18.61 | A | C |
| ATOM | 598 | CD1 | PHE | A | 81 | 7.599 | −52.404 | −11.065 | 1.00 | 18.00 | A | C |
| ATOM | 599 | CE1 | PHE | A | 81 | 7.632 | −51.587 | −12.184 | 1.00 | 18.22 | A | C |
| ATOM | 600 | CZ | PHE | A | 81 | 6.510 | −50.862 | −12.542 | 1.00 | 18.35 | A | C |
| ATOM | 601 | CE2 | PHE | A | 81 | 5.354 | −50.955 | −11.759 | 1.00 | 18.91 | A | C |
| ATOM | 602 | CD2 | PHE | A | 81 | 5.335 | −51.770 | −10.643 | 1.00 | 18.40 | A | C |
| ATOM | 603 | C | PHE | A | 81 | 6.655 | −55.584 | −8.054 | 1.00 | 16.49 | A | C |
| ATOM | 604 | O | PHE | A | 81 | 7.232 | −55.247 | −7.031 | 1.00 | 15.88 | A | O |
| ATOM | 605 | N | GLY | A | 82 | 5.840 | −56.626 | −8.098 | 1.00 | 15.73 | A | N |
| ATOM | 606 | CA | GLY | A | 82 | 5.448 | −57.296 | −6.856 | 1.00 | 15.78 | A | C |
| ATOM | 607 | C | GLY | A | 82 | 4.790 | −56.346 | −5.834 | 1.00 | 15.32 | A | C |
| ATOM | 608 | O | GLY | A | 82 | 4.948 | −56.516 | −4.643 | 1.00 | 15.40 | A | O |
| ATOM | 609 | N | ARG | A | 83 | 4.057 | −55.343 | −6.311 | 1.00 | 14.39 | A | N |
| ATOM | 610 | CA | ARG | A | 83 | 3.366 | −54.424 | −5.447 | 1.00 | 13.68 | A | C |
| ATOM | 611 | CB | ARG | A | 83 | 3.555 | −52.995 | −5.912 | 1.00 | 13.45 | A | C |
| ATOM | 612 | CG | ARG | A | 83 | 3.011 | −52.011 | −4.904 | 1.00 | 13.06 | A | C |
| ATOM | 613 | CD | ARG | A | 83 | 2.913 | −50.629 | −5.479 | 1.00 | 13.28 | A | C |
| ATOM | 614 | NE | ARG | A | 83 | 2.721 | −49.736 | −4.358 | 1.00 | 13.46 | A | N |
| ATOM | 615 | CZ | ARG | A | 83 | 3.697 | −49.171 | −3.662 | 1.00 | 13.30 | A | C |
| ATOM | 616 | NH1 | ARG | A | 83 | 4.971 | −49.321 | −3.998 | 1.00 | 13.23 | A | N |
| ATOM | 617 | NH2 | ARG | A | 83 | 3.375 | −48.419 | −2.631 | 1.00 | 13.54 | A | N |
| ATOM | 618 | C | ARG | A | 83 | 1.871 | −54.704 | −5.384 | 1.00 | 13.51 | A | C |
| ATOM | 619 | O | ARG | A | 83 | 1.183 | −54.725 | −6.390 | 1.00 | 13.70 | A | O |
| ATOM | 620 | N | ASN | A | 84 | 1.390 | −54.879 | −4.167 | 1.00 | 13.56 | A | N |
| ATOM | 621 | CA | ASN | A | 84 | 0.009 | −55.148 | −3.893 | 1.00 | 13.51 | A | C |
| ATOM | 622 | CB | ASN | A | 84 | −0.088 | −56.018 | −2.658 | 1.00 | 13.59 | A | C |
| ATOM | 623 | CG | ASN | A | 84 | −1.491 | −56.487 | −2.384 | 1.00 | 14.02 | A | C |
| ATOM | 624 | OD1 | ASN | A | 84 | −1.725 | −57.145 | −1.381 | 1.00 | 15.08 | A | O |
| ATOM | 625 | ND2 | ASN | A | 84 | −2.428 | −56.165 | −3.261 | 1.00 | 13.79 | A | N |
| ATOM | 626 | C | ASN | A | 84 | −0.760 | −53.864 | −3.630 | 1.00 | 13.38 | A | C |
| ATOM | 627 | O | ASN | A | 84 | −0.857 | −53.447 | −2.476 | 1.00 | 13.02 | A | O |
| ATOM | 628 | N | SER | A | 85 | −1.301 | −53.271 | −4.707 | 1.00 | 12.63 | A | N |
| ATOM | 629 | CA | SER | A | 85 | −2.121 | −52.055 | −4.669 | 1.00 | 11.89 | A | C |
| ATOM | 630 | CB | SER | A | 85 | −3.343 | −52.185 | −3.746 | 1.00 | 11.20 | A | C |
| ATOM | 631 | OG | SER | A | 85 | −4.254 | −51.134 | −3.980 | 1.00 | 10.14 | A | O |
| ATOM | 632 | C | SER | A | 85 | −1.291 | −50.867 | −4.273 | 1.00 | 12.38 | A | C |
| ATOM | 633 | O | SER | A | 85 | −0.066 | −50.944 | −4.210 | 1.00 | 12.63 | A | O |
| ATOM | 634 | N | ILE | A | 86 | −1.990 | −49.773 | −3.990 | 1.00 | 12.96 | A | N |
| ATOM | 635 | CA | ILE | A | 86 | −1.396 | −48.453 | −3.822 | 1.00 | 13.20 | A | C |
| ATOM | 636 | CB | ILE | A | 86 | −2.512 | −47.385 | −3.647 | 1.00 | 13.89 | A | C |
| ATOM | 637 | CG1 | ILE | A | 86 | −3.144 | −47.100 | −5.011 | 1.00 | 13.93 | A | C |
| ATOM | 638 | CD1 | ILE | A | 86 | −4.318 | −46.147 | −4.967 | 1.00 | 14.24 | A | C |
| ATOM | 639 | CG2 | ILE | A | 86 | −1.996 | −46.082 | −3.034 | 1.00 | 14.48 | A | C |
| ATOM | 640 | C | ILE | A | 86 | −0.431 | −48.423 | −2.663 | 1.00 | 13.17 | A | C |
| ATOM | 641 | O | ILE | A | 86 | 0.620 | −47.786 | −2.759 | 1.00 | 13.10 | A | O |
| ATOM | 642 | N | ASP | A | 87 | −0.767 | −49.114 | −1.569 | 1.00 | 12.99 | A | N |
| ATOM | 643 | CA | ASP | A | 87 | 0.130 | −49.095 | −0.380 | 1.00 | 12.59 | A | C |
| ATOM | 644 | CB | ASP | A | 87 | −0.644 | −48.780 | 0.920 | 1.00 | 12.29 | A | C |
| ATOM | 645 | CG | ASP | A | 87 | −1.666 | −49.810 | 1.265 | 1.00 | 11.98 | A | C |
| ATOM | 646 | OD1 | ASP | A | 87 | −1.658 | −50.921 | 0.683 | 1.00 | 12.11 | A | O |
| ATOM | 647 | OD2 | ASP | A | 87 | −2.496 | −49.479 | 2.115 | 1.00 | 11.43 | A | O |
| ATOM | 648 | C | ASP | A | 87 | 1.028 | −50.320 | −0.225 | 1.00 | 12.13 | A | C |
| ATOM | 649 | O | ASP | A | 87 | 1.692 | −50.494 | 0.782 | 1.00 | 11.57 | A | O |
| ATOM | 650 | N | GLY | A | 88 | 1.067 | −51.168 | −1.236 | 1.00 | 12.40 | A | N |
| ATOM | 651 | CA | GLY | A | 88 | 1.807 | −52.407 | −1.092 | 1.00 | 12.89 | A | C |
| ATOM | 652 | C | GLY | A | 88 | 1.201 | −53.421 | −0.146 | 1.00 | 13.24 | A | C |
| ATOM | 653 | O | GLY | A | 88 | 1.699 | −54.542 | −0.132 | 1.00 | 14.52 | A | O |
| ATOM | 654 | N | ASN | A | 89 | 0.115 | −53.051 | 0.567 | 1.00 | 12.96 | A | N |
| ATOM | 655 | CA | ASN | A | 89 | −0.658 | −53.912 | 1.473 | 1.00 | 12.96 | A | C |
| ATOM | 656 | CB | ASN | A | 89 | −0.669 | −53.249 | 2.844 | 1.00 | 13.56 | A | C |
| ATOM | 657 | CG | ASN | A | 89 | 0.641 | −53.362 | 3.537 | 1.00 | 13.79 | A | C |
| ATOM | 658 | OD1 | ASN | A | 89 | 1.205 | −54.457 | 3.631 | 1.00 | 14.84 | A | O |
| ATOM | 659 | ND2 | ASN | A | 89 | 1.149 | −52.242 | 4.029 | 1.00 | 13.75 | A | N |
| ATOM | 660 | C | ASN | A | 89 | −2.147 | −54.210 | 1.165 | 1.00 | 12.88 | A | C |
| ATOM | 661 | O | ASN | A | 89 | −2.907 | −54.462 | 2.095 | 1.00 | 12.88 | A | O |
| ATOM | 662 | N | GLY | A | 90 | −2.574 | −54.175 | −0.097 | 1.00 | 12.86 | A | N |
| ATOM | 663 | CA | GLY | A | 90 | −3.958 | −54.471 | −0.469 | 1.00 | 12.39 | A | C |
| ATOM | 664 | C | GLY | A | 90 | −4.961 | −53.302 | −0.474 | 1.00 | 12.26 | A | C |
| ATOM | 665 | O | GLY | A | 90 | −6.137 | −53.514 | −0.791 | 1.00 | 12.36 | A | O |
| ATOM | 666 | N | PHE | A | 91 | −4.527 | −52.091 | −0.118 | 1.00 | 11.47 | A | N |
| ATOM | 667 | CA | PHE | A | 91 | −5.390 | −50.906 | −0.131 | 1.00 | 11.28 | A | C |
| ATOM | 668 | CB | PHE | A | 91 | −4.545 | −49.680 | −0.502 | 1.00 | 11.72 | A | C |
| ATOM | 669 | CG | PHE | A | 91 | −5.262 | −48.333 | −0.429 | 1.00 | 11.82 | A | C |
| ATOM | 670 | CD1 | PHE | A | 91 | −6.027 | −47.869 | −1.503 | 1.00 | 11.80 | A | C |
| ATOM | 671 | CE1 | PHE | A | 91 | −6.634 | −46.609 | −1.454 | 1.00 | 11.78 | A | C |
| ATOM | 672 | CZ | PHE | A | 91 | −6.457 | −45.791 | −0.341 | 1.00 | 11.56 | A | C |
| ATOM | 673 | CE2 | PHE | A | 91 | −5.647 | −46.217 | 0.706 | 1.00 | 11.63 | A | C |
| ATOM | 674 | CD2 | PHE | A | 91 | −5.053 | −47.470 | 0.663 | 1.00 | 11.62 | A | C |
| ATOM | 675 | C | PHE | A | 91 | −6.509 | −51.027 | −1.132 | 1.00 | 10.82 | A | C |
| ATOM | 676 | O | PHE | A | 91 | −6.262 | −50.984 | −2.306 | 1.00 | 11.40 | A | O |
| ATOM | 677 | N | GLN | A | 92 | −7.739 | −51.128 | −0.659 | 1.00 | 10.46 | A | N |
| ATOM | 678 | CA | GLN | A | 92 | −8.903 | −51.300 | −1.520 | 1.00 | 10.16 | A | C |
| ATOM | 679 | CB | GLN | A | 92 | −10.190 | −51.348 | −0.665 | 1.00 | 9.81 | A | C |
| ATOM | 680 | CG | GLN | A | 92 | −11.435 | −51.789 | −1.424 | 1.00 | 9.40 | A | C |
| ATOM | 681 | CD | GLN | A | 92 | −12.658 | −51.830 | −0.560 | 1.00 | 9.00 | A | C |
| ATOM | 682 | OE1 | GLN | A | 92 | −13.251 | −50.800 | −0.244 | 1.00 | 8.91 | A | O |
| ATOM | 683 | NE2 | GLN | A | 92 | −13.056 | −53.023 | −0.179 | 1.00 | 8.85 | A | N |
| ATOM | 684 | C | GLN | A | 92 | −9.020 | −50.172 | −2.547 | 1.00 | 10.41 | A | C |
| ATOM | 685 | O | GLN | A | 92 | −8.852 | −49.014 | −2.220 | 1.00 | 10.73 | A | O |
| ATOM | 686 | N | LEU | A | 93 | −9.372 | −50.531 | −3.774 | 1.00 | 10.45 | A | N |
| ATOM | 687 | CA | LEU | A | 93 | −9.461 | −49.608 | −4.873 | 1.00 | 10.53 | A | C |
| ATOM | 688 | CB | LEU | A | 93 | −8.795 | −50.248 | −6.088 | 1.00 | 10.43 | A | C |
| ATOM | 689 | CG | LEU | A | 93 | −7.325 | −50.530 | −5.783 | 1.00 | 10.45 | A | C |
| ATOM | 690 | CD1 | LEU | A | 93 | −6.629 | −51.095 | −6.991 | 1.00 | 10.58 | A | C |
| ATOM | 691 | CD2 | LEU | A | 93 | −6.606 | −49.286 | −5.321 | 1.00 | 10.42 | A | C |
| ATOM | 692 | C | LEU | A | 93 | −10.894 | −49.254 | −5.222 | 1.00 | 10.87 | A | C |
| ATOM | 693 | O | LEU | A | 93 | −11.686 | −50.097 | −5.637 | 1.00 | 10.94 | A | O |
| ATOM | 694 | N | LYS | A | 94 | −11.228 | −47.996 | −5.085 | 1.00 | 11.36 | A | N |
| ATOM | 695 | CA | LYS | A | 94 | −12.557 | −47.606 | −5.384 | 1.00 | 12.41 | A | C |
| ATOM | 696 | CB | LYS | A | 94 | −13.181 | −46.937 | −4.197 | 1.00 | 13.22 | A | C |
| ATOM | 697 | CG | LYS | A | 94 | −13.478 | −47.903 | −3.087 | 1.00 | 13.81 | A | C |
| ATOM | 698 | CD | LYS | A | 94 | −14.287 | −47.225 | −1.995 | 1.00 | 14.67 | A | C |
| ATOM | 699 | CE | LYS | A | 94 | −13.694 | −47.606 | −0.647 | 1.00 | 15.89 | A | C |
| ATOM | 700 | NZ | LYS | A | 94 | −14.804 | −47.787 | 0.313 | 1.00 | 16.73 | A | N |
| ATOM | 701 | C | LYS | A | 94 | −12.553 | −46.638 | −6.490 | 1.00 | 12.80 | A | C |
| ATOM | 702 | O | LYS | A | 94 | −11.826 | −45.662 | −6.437 | 1.00 | 14.03 | A | O |
| ATOM | 703 | N | SER | A | 95 | −13.419 | −46.878 | −7.462 | 1.00 | 12.71 | A | N |
| ATOM | 704 | CA | SER | A | 95 | −13.544 | −46.022 | −8.613 | 1.00 | 12.55 | A | C |
| ATOM | 705 | CB | SER | A | 95 | −13.161 | −46.813 | −9.861 | 1.00 | 12.73 | A | C |
| ATOM | 706 | OG | SER | A | 95 | −11.804 | −47.234 | −9.800 | 1.00 | 12.59 | A | O |
| ATOM | 707 | C | SER | A | 95 | −14.974 | −45.525 | −8.688 | 1.00 | 12.34 | A | C |
| ATOM | 708 | O | SER | A | 95 | −15.918 | −46.275 | −8.456 | 1.00 | 12.56 | A | O |
| ATOM | 709 | N | THR | A | 96 | −15.138 | −44.241 | −8.962 | 1.00 | 12.35 | A | N |
| ATOM | 710 | CA | THR | A | 96 | −16.466 | −43.667 | −9.154 | 1.00 | 12.18 | A | C |
| ATOM | 711 | CB | THR | A | 96 | −16.765 | −42.598 | −8.118 | 1.00 | 12.24 | A | C |
| ATOM | 712 | OG1 | THR | A | 96 | −16.552 | −43.158 | −6.828 | 1.00 | 12.21 | A | O |
| ATOM | 713 | CG2 | THR | A | 96 | −18.231 | −42.087 | −8.247 | 1.00 | 12.24 | A | C |
| ATOM | 714 | C | THR | A | 96 | −16.560 | −42.995 | −10.490 | 1.00 | 12.02 | A | C |
| ATOM | 715 | O | THR | A | 96 | −15.804 | −42.073 | −10.768 | 1.00 | 11.98 | A | O |
| ATOM | 716 | N | VAL | A | 97 | −17.512 | −43.453 | −11.286 | 1.00 | 12.10 | A | N |
| ATOM | 717 | CA | VAL | A | 97 | −17.796 | −42.903 | −12.610 | 1.00 | 12.29 | A | C |
| ATOM | 718 | CB | VAL | A | 97 | −18.031 | −44.046 | −13.614 | 1.00 | 11.84 | A | C |
| ATOM | 719 | CG1 | VAL | A | 97 | −16.842 | −44.997 | −13.607 | 1.00 | 11.85 | A | C |
| ATOM | 720 | CG2 | VAL | A | 97 | −19.294 | −44.816 | −13.291 | 1.00 | 11.48 | A | C |
| ATOM | 721 | C | VAL | A | 97 | −19.043 | −41.989 | −12.574 | 1.00 | 12.50 | A | C |
| ATOM | 722 | O | VAL | A | 97 | −19.760 | −41.922 | −11.573 | 1.00 | 12.20 | A | O |
| ATOM | 723 | N | HIS | A | 98 | −19.307 | −41.321 | −13.693 | 1.00 | 12.79 | A | N |
| ATOM | 724 | CA | HIS | A | 98 | −20.400 | −40.338 | −13.803 | 1.00 | 12.70 | A | C |
| ATOM | 725 | CB | HIS | A | 98 | −21.764 | −40.997 | −13.788 | 1.00 | 12.30 | A | C |
| ATOM | 726 | CG | HIS | A | 98 | −21.870 | −42.180 | −14.687 | 1.00 | 11.98 | A | C |
| ATOM | 727 | ND1 | HIS | A | 98 | −21.648 | −42.103 | −16.040 | 1.00 | 11.73 | A | N |
| ATOM | 728 | CE1 | HIS | A | 98 | −21.815 | −43.295 | −16.574 | 1.00 | 11.81 | A | C |
| ATOM | 729 | NE2 | HIS | A | 98 | −22.147 | −44.138 | −15.617 | 1.00 | 11.79 | A | N |
| ATOM | 730 | CD2 | HIS | A | 98 | −22.206 | −43.463 | −14.429 | 1.00 | 11.79 | A | C |
| ATOM | 731 | C | HIS | A | 98 | −20.334 | −39.328 | −12.685 | 1.00 | 13.21 | A | C |
| ATOM | 732 | O | HIS | A | 98 | −21.348 | −38.988 | −12.091 | 1.00 | 13.62 | A | O |
| ATOM | 733 | N | TYR | A | 99 | −19.117 | −38.883 | −12.389 | 1.00 | 13.68 | A | N |
| ATOM | 734 | CA | TYR | A | 99 | −18.894 | −37.826 | −11.446 | 1.00 | 13.61 | A | C |
| ATOM | 735 | CB | TYR | A | 99 | −17.410 | −37.665 | −11.086 | 1.00 | 13.46 | A | C |
| ATOM | 736 | CG | TYR | A | 99 | −17.177 | −36.565 | −10.042 | 1.00 | 13.94 | A | C |
| ATOM | 737 | CD1 | TYR | A | 99 | −17.426 | −36.805 | −8.691 | 1.00 | 14.10 | A | C |
| ATOM | 738 | CE1 | TYR | A | 99 | −17.213 | −35.826 | −7.731 | 1.00 | 13.91 | A | C |
| ATOM | 739 | CZ | TYR | A | 99 | −16.774 | −34.579 | −8.108 | 1.00 | 13.62 | A | C |
| ATOM | 740 | OH | TYR | A | 99 | −16.605 | −33.644 | −7.112 | 1.00 | 12.97 | A | O |
| ATOM | 741 | CE2 | TYR | A | 99 | −16.553 | −34.292 | −9.445 | 1.00 | 13.47 | A | C |
| ATOM | 742 | CD2 | TYR | A | 99 | −16.756 | −35.278 | −10.400 | 1.00 | 13.78 | A | C |
| ATOM | 743 | C | TYR | A | 99 | −19.384 | −36.564 | −12.086 | 1.00 | 13.49 | A | C |
| ATOM | 744 | O | TYR | A | 99 | −18.870 | −36.163 | −13.106 | 1.00 | 12.90 | A | O |
| ATOM | 745 | N | SER | A | 100 | −20.399 | −35.974 | −11.471 | 1.00 | 14.43 | A | N |
| ATOM | 746 | CA | SER | A | 100 | −20.823 | −34.601 | −11.733 | 1.00 | 15.24 | A | C |
| ATOM | 747 | CB | SER | A | 100 | −19.639 | −33.661 | −11.530 | 1.00 | 15.55 | A | C |
| ATOM | 748 | OG | SER | A | 100 | −20.037 | −32.309 | −11.482 | 1.00 | 15.70 | A | O |
| ATOM | 749 | C | SER | A | 100 | −21.440 | −34.469 | −13.114 | 1.00 | 16.08 | A | C |
| ATOM | 750 | O | SER | A | 100 | −21.916 | −35.443 | −13.679 | 1.00 | 16.02 | A | O |
| ATOM | 751 | N | SER | A | 101 | −21.448 | −33.271 | −13.668 | 1.00 | 18.00 | A | N |
| ATOM | 752 | CA | SER | A | 101 | −22.109 | −33.064 | −14.954 | 1.00 | 20.04 | A | C |
| ATOM | 753 | CB | SER | A | 101 | −23.285 | −32.117 | −14.785 | 1.00 | 20.79 | A | C |
| ATOM | 754 | OG | SER | A | 101 | −24.125 | −32.255 | −15.909 | 1.00 | 22.68 | A | O |
| ATOM | 755 | C | SER | A | 101 | −21.172 | −32.575 | −16.074 | 1.00 | 20.29 | A | C |
| ATOM | 756 | O | SER | A | 101 | −20.448 | −31.590 | −15.909 | 1.00 | 18.68 | A | O |
| ATOM | 757 | N | ARG | A | 102 | −21.210 | −33.275 | −17.212 | 1.00 | 21.31 | A | N |
| ATOM | 758 | CA | ARG | A | 102 | −20.354 | −32.962 | −18.351 | 1.00 | 22.92 | A | C |
| ATOM | 759 | CB | ARG | A | 102 | −20.973 | −31.842 | −19.222 | 1.00 | 25.54 | A | C |
| ATOM | 760 | CG | ARG | A | 102 | −22.375 | −32.170 | −19.771 | 1.00 | 29.36 | A | C |
| ATOM | 761 | CD | ARG | A | 102 | −22.597 | −31.658 | −21.202 | 1.00 | 32.59 | A | C |
| ATOM | 762 | NE | ARG | A | 102 | −22.425 | −30.196 | −21.266 | 1.00 | 35.58 | A | N |
| ATOM | 763 | CZ | ARG | A | 102 | −21.863 | −29.501 | −22.268 | 1.00 | 38.08 | A | C |
| ATOM | 764 | NH1 | ARG | A | 102 | −21.371 | −30.080 | −23.375 | 1.00 | 37.26 | A | N |
| ATOM | 765 | NH2 | ARG | A | 102 | −21.785 | −28.183 | −22.160 | 1.00 | 40.05 | A | N |
| ATOM | 766 | C | ARG | A | 102 | −18.972 | −32.558 | −17.854 | 1.00 | 21.22 | A | C |
| ATOM | 767 | O | ARG | A | 102 | −18.499 | −31.478 | −18.155 | 1.00 | 22.96 | A | O |
| ATOM | 768 | N | TYR | A | 103 | −18.337 | −33.431 | −17.088 | 1.00 | 19.27 | A | N |
| ATOM | 769 | CA | TYR | A | 103 | −17.131 | −33.068 | −16.304 | 1.00 | 18.16 | A | C |
| ATOM | 770 | CB | TYR | A | 103 | −17.246 | −33.653 | −14.876 | 1.00 | 17.43 | A | C |
| ATOM | 771 | CG | TYR | A | 103 | −16.076 | −33.397 | −14.001 | 1.00 | 17.19 | A | C |
| ATOM | 772 | CD1 | TYR | A | 103 | −15.957 | −32.216 | −13.283 | 1.00 | 18.00 | A | C |
| ATOM | 773 | CE1 | TYR | A | 103 | −14.860 | −31.991 | −12.450 | 1.00 | 18.06 | A | C |
| ATOM | 774 | CZ | TYR | A | 103 | −13.875 | −32.964 | −12.348 | 1.00 | 17.35 | A | C |
| ATOM | 775 | OH | TYR | A | 103 | −12.774 | −32.784 | −11.574 | 1.00 | 17.41 | A | O |
| ATOM | 776 | CE2 | TYR | A | 103 | −13.985 | −34.135 | −13.042 | 1.00 | 17.27 | A | C |
| ATOM | 777 | CD2 | TYR | A | 103 | −15.078 | −34.345 | −13.864 | 1.00 | 17.58 | A | C |
| ATOM | 778 | C | TYR | A | 103 | −15.854 | −33.534 | −17.030 | 1.00 | 16.89 | A | C |
| ATOM | 779 | O | TYR | A | 103 | −15.682 | −34.721 | −17.328 | 1.00 | 16.93 | A | O |
| ATOM | 780 | N | ASN | A | 104 | −14.967 | −32.600 | −17.341 | 1.00 | 15.51 | A | N |
| ATOM | 781 | CA | ASN | A | 104 | −13.893 | −32.910 | −18.257 | 1.00 | 14.63 | A | C |
| ATOM | 782 | CB | ASN | A | 104 | −13.665 | −31.743 | −19.205 | 1.00 | 14.81 | A | C |
| ATOM | 783 | CG | ASN | A | 104 | −14.612 | −31.750 | −20.400 | 1.00 | 15.08 | A | C |
| ATOM | 784 | OD1 | ASN | A | 104 | −15.024 | −32.796 | −20.896 | 1.00 | 15.19 | A | O |
| ATOM | 785 | ND2 | ASN | A | 104 | −14.920 | −30.571 | −20.894 | 1.00 | 14.99 | A | N |
| ATOM | 786 | C | ASN | A | 104 | −12.614 | −33.257 | −17.504 | 1.00 | 13.94 | A | C |
| ATOM | 787 | O | ASN | A | 104 | −11.578 | −32.636 | −17.730 | 1.00 | 13.92 | A | O |
| ATOM | 788 | N | ASN | A | 105 | −12.697 | −34.257 | −16.616 | 1.00 | 12.63 | A | N |
| ATOM | 789 | CA | ASN | A | 105 | −11.555 | −34.707 | −15.844 | 1.00 | 11.74 | A | C |
| ATOM | 790 | CB | ASN | A | 105 | −11.145 | −33.649 | −14.826 | 1.00 | 11.60 | A | C |
| ATOM | 791 | CG | ASN | A | 105 | −9.690 | −33.277 | −14.949 | 1.00 | 11.53 | A | C |
| ATOM | 792 | OD1 | ASN | A | 105 | −8.832 | −34.125 | −15.116 | 1.00 | 11.68 | A | O |
| ATOM | 793 | ND2 | ASN | A | 105 | −9.410 | −32.009 | −14.887 | 1.00 | 11.47 | A | N |
| ATOM | 794 | C | ASN | A | 105 | −11.738 | −36.035 | −15.116 | 1.00 | 11.37 | A | C |
| ATOM | 795 | O | ASN | A | 105 | −12.840 | −36.575 | −15.031 | 1.00 | 11.26 | A | O |
| ATOM | 796 | N | ALA | A | 106 | −10.614 | −36.562 | −14.637 | 1.00 | 11.03 | A | N |
| ATOM | 797 | CA | ALA | A | 106 | −10.579 | −37.638 | −13.650 | 1.00 | 10.62 | A | C |
| ATOM | 798 | CB | ALA | A | 106 | −10.233 | −38.958 | −14.290 | 1.00 | 10.51 | A | C |
| ATOM | 799 | C | ALA | A | 106 | −9.512 | −37.269 | −12.681 | 1.00 | 10.35 | A | C |
| ATOM | 800 | O | ALA | A | 106 | −8.663 | −36.428 | −12.992 | 1.00 | 10.10 | A | O |
| ATOM | 801 | N | PHE | A | 107 | −9.522 | −37.920 | −11.525 | 1.00 | 10.44 | A | N |
| ATOM | 802 | CA | PHE | A | 107 | −8.637 | −37.527 | −10.431 | 1.00 | 10.55 | A | C |
| ATOM | 803 | CB | PHE | A | 107 | −8.983 | −36.124 | −9.903 | 1.00 | 10.32 | A | C |
| ATOM | 804 | CG | PHE | A | 107 | −10.352 | −36.015 | −9.323 | 1.00 | 10.51 | A | C |
| ATOM | 805 | CD2 | PHE | A | 107 | −11.421 | −35.620 | −10.104 | 1.00 | 10.75 | A | C |
| ATOM | 806 | CE2 | PHE | A | 107 | −12.694 | −35.504 | −9.561 | 1.00 | 10.83 | A | C |
| ATOM | 807 | CZ | PHE | A | 107 | −12.905 | −35.812 | −8.233 | 1.00 | 10.89 | A | C |
| ATOM | 808 | CE1 | PHE | A | 107 | −11.841 | −36.197 | −7.445 | 1.00 | 10.78 | A | C |
| ATOM | 809 | CD1 | PHE | A | 107 | −10.575 | −36.280 | −7.994 | 1.00 | 10.79 | A | C |
| ATOM | 810 | C | PHE | A | 107 | −8.568 | −38.494 | −9.258 | 1.00 | 10.67 | A | C |
| ATOM | 811 | O | PHE | A | 107 | −9.372 | −39.418 | −9.136 | 1.00 | 10.36 | A | O |
| ATOM | 812 | N | TRP | A | 108 | −7.575 | −38.222 | −8.404 | 1.00 | 11.14 | A | N |
| ATOM | 813 | CA | TRP | A | 108 | −7.276 | −38.991 | −7.221 | 1.00 | 11.55 | A | C |
| ATOM | 814 | CB | TRP | A | 108 | −5.895 | −39.634 | −7.328 | 1.00 | 11.58 | A | C |
| ATOM | 815 | CG | TRP | A | 108 | −5.269 | −39.991 | −6.019 | 1.00 | 11.70 | A | C |
| ATOM | 816 | CD1 | TRP | A | 108 | −4.276 | −39.320 | −5.372 | 1.00 | 11.59 | A | C |
| ATOM | 817 | NE1 | TRP | A | 108 | −3.967 | −39.954 | −4.192 | 1.00 | 11.77 | A | N |
| ATOM | 818 | CE2 | TRP | A | 108 | −4.765 | −41.058 | −4.059 | 1.00 | 11.81 | A | C |
| ATOM | 819 | CD2 | TRP | A | 108 | −5.600 | −41.113 | −5.189 | 1.00 | 12.15 | A | C |
| ATOM | 820 | CE3 | TRP | A | 108 | −6.526 | −42.169 | −5.295 | 1.00 | 12.29 | A | C |
| ATOM | 821 | CZ3 | TRP | A | 108 | −6.583 | −43.109 | −4.277 | 1.00 | 11.79 | A | C |
| ATOM | 822 | CH2 | TRP | A | 108 | −5.736 | −43.017 | −3.178 | 1.00 | 11.69 | A | C |
| ATOM | 823 | CZ2 | TRP | A | 108 | −4.827 | −42.005 | −3.049 | 1.00 | 11.78 | A | C |
| ATOM | 824 | C | TRP | A | 108 | −7.301 | −38.046 | −6.058 | 1.00 | 11.89 | A | C |
| ATOM | 825 | O | TRP | A | 108 | −6.541 | −37.070 | −6.011 | 1.00 | 12.66 | A | O |
| ATOM | 826 | N | ASN | A | 109 | −8.146 | −38.361 | −5.086 | 1.00 | 12.02 | A | N |
| ATOM | 827 | CA | ASN | A | 109 | −8.413 | −37.441 | −3.998 | 1.00 | 11.55 | A | C |
| ATOM | 828 | CB | ASN | A | 109 | −9.910 | −37.250 | −3.866 | 1.00 | 10.98 | A | C |
| ATOM | 829 | CG | ASN | A | 109 | −10.615 | −38.456 | −3.291 | 1.00 | 10.78 | A | C |
| ATOM | 830 | OD1 | ASN | A | 109 | −10.027 | −39.428 | −2.857 | 1.00 | 9.78 | A | O |
| ATOM | 831 | ND2 | ASN | A | 109 | −11.918 | −38.378 | −3.297 | 1.00 | 11.23 | A | N |
| ATOM | 832 | C | ASN | A | 109 | −7.821 | −37.800 | −2.637 | 1.00 | 11.83 | A | C |
| ATOM | 833 | O | ASN | A | 109 | −8.205 | −37.180 | −1.653 | 1.00 | 12.98 | A | O |
| ATOM | 834 | N | GLY | A | 110 | −6.911 | −38.767 | −2.566 | 1.00 | 11.58 | A | N |
| ATOM | 835 | CA | GLY | A | 110 | −6.353 | −39.211 | −1.286 | 1.00 | 11.33 | A | C |
| ATOM | 836 | C | GLY | A | 110 | −6.978 | −40.526 | −0.825 | 1.00 | 11.37 | A | C |
| ATOM | 837 | O | GLY | A | 110 | −6.430 | −41.220 | 0.014 | 1.00 | 12.11 | A | O |
| ATOM | 838 | N | VAL | A | 111 | −8.105 | −40.898 | −1.404 | 1.00 | 11.10 | A | N |
| ATOM | 839 | CA | VAL | A | 111 | −8.926 | −42.006 | −0.912 | 1.00 | 10.97 | A | C |
| ATOM | 840 | CB | VAL | A | 111 | −10.193 | −41.411 | −0.186 | 1.00 | 11.34 | A | C |
| ATOM | 841 | CG1 | VAL | A | 111 | −11.387 | −42.362 | −0.056 | 1.00 | 11.21 | A | C |
| ATOM | 842 | CG2 | VAL | A | 111 | −9.789 | −40.889 | 1.181 | 1.00 | 11.73 | A | C |
| ATOM | 843 | C | VAL | A | 111 | −9.306 | −42.918 | −2.065 | 1.00 | 10.59 | A | C |
| ATOM | 844 | O | VAL | A | 111 | −9.366 | −44.119 | −1.902 | 1.00 | 10.53 | A | O |
| ATOM | 845 | N | GLN | A | 112 | −9.574 | −42.345 | −3.227 | 1.00 | 10.74 | A | N |
| ATOM | 846 | CA | GLN | A | 112 | −10.166 | −43.079 | −4.322 | 1.00 | 10.93 | A | C |
| ATOM | 847 | CB | GLN | A | 112 | −11.652 | −43.280 | −4.029 | 1.00 | 10.80 | A | C |
| ATOM | 848 | CG | GLN | A | 112 | −12.502 | −42.023 | −4.050 | 1.00 | 10.59 | A | C |
| ATOM | 849 | CD | GLN | A | 112 | −13.930 | −42.371 | −4.436 | 1.00 | 10.69 | A | C |
| ATOM | 850 | OE1 | GLN | A | 112 | −14.867 | −42.252 | −3.631 | 1.00 | 10.62 | A | O |
| ATOM | 851 | NE2 | GLN | A | 112 | −14.092 | −42.878 | −5.659 | 1.00 | 10.64 | A | N |
| ATOM | 852 | C | GLN | A | 112 | −10.020 | −42.410 | −5.681 | 1.00 | 11.08 | A | C |
| ATOM | 853 | O | GLN | A | 112 | −9.555 | −41.283 | −5.792 | 1.00 | 10.83 | A | O |
| ATOM | 854 | N | MET | A | 113 | −10.449 | −43.117 | −6.713 | 1.00 | 11.57 | A | N |
| ATOM | 855 | CA | MET | A | 113 | −10.386 | −42.607 | −8.068 | 1.00 | 12.22 | A | C |
| ATOM | 856 | CB | MET | A | 113 | −9.770 | −43.655 | −9.012 | 1.00 | 12.75 | A | C |
| ATOM | 857 | CG | MET | A | 113 | −8.248 | −43.793 | −8.921 | 1.00 | 12.88 | A | C |
| ATOM | 858 | SD | MET | A | 113 | −7.704 | −44.724 | −7.465 | 1.00 | 13.61 | A | S |
| ATOM | 859 | CE | MET | A | 113 | −8.484 | −46.336 | −7.684 | 1.00 | 12.88 | A | C |
| ATOM | 860 | C | MET | A | 113 | −11.779 | −42.229 | −8.566 | 1.00 | 12.30 | A | C |
| ATOM | 861 | O | MET | A | 113 | −12.777 | −42.930 | −8.298 | 1.00 | 11.89 | A | O |
| ATOM | 862 | N | VAL | A | 114 | −11.813 | −41.138 | −9.338 | 1.00 | 12.45 | A | N |
| ATOM | 863 | CA | VAL | A | 114 | −13.060 | −40.524 | −9.829 | 1.00 | 12.39 | A | C |
| ATOM | 864 | CB | VAL | A | 114 | −13.312 | −39.200 | −9.108 | 1.00 | 12.60 | A | C |
| ATOM | 865 | CG1 | VAL | A | 114 | −14.735 | −38.756 | −9.269 | 1.00 | 12.58 | A | C |
| ATOM | 866 | CG2 | VAL | A | 114 | −13.006 | −39.354 | −7.641 | 1.00 | 13.14 | A | C |
| ATOM | 867 | C | VAL | A | 114 | −12.892 | −40.165 | −11.291 | 1.00 | 11.82 | A | C |
| ATOM | 868 | O | VAL | A | 114 | −11.824 | −39.682 | −11.672 | 1.00 | 11.70 | A | O |
| ATOM | 869 | N | TYR | A | 115 | −13.943 | −40.368 | −12.085 | 1.00 | 11.01 | A | N |
| ATOM | 870 | CA | TYR | A | 115 | −13.868 | −40.205 | −13.517 | 1.00 | 10.70 | A | C |
| ATOM | 871 | CB | TYR | A | 115 | −13.724 | −41.578 | −14.192 | 1.00 | 10.58 | A | C |
| ATOM | 872 | CG | TYR | A | 115 | −12.538 | −42.408 | −13.680 | 1.00 | 10.64 | A | C |
| ATOM | 873 | CD1 | TYR | A | 115 | −11.258 | −42.218 | −14.179 | 1.00 | 10.65 | A | C |
| ATOM | 874 | CE1 | TYR | A | 115 | −10.189 | −42.936 | −13.703 | 1.00 | 10.73 | A | C |
| ATOM | 875 | CZ | TYR | A | 115 | −10.376 | −43.877 | −12.724 | 1.00 | 11.06 | A | C |
| ATOM | 876 | OH | TYR | A | 115 | −9.298 | −44.623 | −12.258 | 1.00 | 11.31 | A | O |
| ATOM | 877 | CE2 | TYR | A | 115 | −11.637 | −44.098 | −12.219 | 1.00 | 10.82 | A | C |
| ATOM | 878 | CD2 | TYR | A | 115 | −12.698 | −43.351 | −12.682 | 1.00 | 10.64 | A | C |
| ATOM | 879 | C | TYR | A | 115 | −15.103 | −39.493 | −14.016 | 1.00 | 10.93 | A | C |
| ATOM | 880 | O | TYR | A | 115 | −16.210 | −40.015 | −13.945 | 1.00 | 11.10 | A | O |
| ATOM | 881 | N | GLY | A | 116 | −14.917 | −38.278 | −14.505 | 1.00 | 11.20 | A | N |
| ATOM | 882 | CA | GLY | A | 116 | −15.952 | −37.601 | −15.247 | 1.00 | 11.38 | A | C |
| ATOM | 883 | C | GLY | A | 116 | −16.368 | −38.324 | −16.522 | 1.00 | 11.63 | A | C |
| ATOM | 884 | O | GLY | A | 116 | −15.747 | −39.265 | −16.955 | 1.00 | 10.90 | A | O |
| ATOM | 885 | N | ASP | A | 117 | −17.456 | −37.839 | −17.105 | 1.00 | 12.56 | A | N |
| ATOM | 886 | CA | ASP | A | 117 | −18.020 | −38.340 | −18.365 | 1.00 | 12.79 | A | C |
| ATOM | 887 | CB | ASP | A | 117 | −19.545 | −38.204 | −18.392 | 1.00 | 12.28 | A | C |
| ATOM | 888 | CG | ASP | A | 117 | −20.245 | −39.348 | −17.701 | 1.00 | 12.14 | A | C |
| ATOM | 889 | OD1 | ASP | A | 117 | −19.932 | −40.511 | −17.976 | 1.00 | 11.76 | A | O |
| ATOM | 890 | OD2 | ASP | A | 117 | −21.149 | −39.081 | −16.895 | 1.00 | 12.59 | A | O |
| ATOM | 891 | C | ASP | A | 117 | −17.509 | −37.570 | −19.541 | 1.00 | 13.10 | A | C |
| ATOM | 892 | O | ASP | A | 117 | −17.586 | −38.054 | −20.648 | 1.00 | 13.73 | A | O |
| ATOM | 893 | N | GLY | A | 118 | −17.039 | −36.356 | −19.301 | 1.00 | 13.57 | A | N |
| ATOM | 894 | CA | GLY | A | 118 | −16.573 | −35.481 | −20.364 | 1.00 | 13.80 | A | C |
| ATOM | 895 | C | GLY | A | 118 | −17.762 | −34.764 | −20.948 | 1.00 | 14.14 | A | C |
| ATOM | 896 | O | GLY | A | 118 | −18.900 | −35.121 | −20.631 | 1.00 | 14.07 | A | O |
| ATOM | 897 | N | ASP | A | 119 | −17.507 | −33.780 | −21.812 | 1.00 | 14.55 | A | N |
| ATOM | 898 | CA | ASP | A | 119 | −18.589 | −32.932 | −22.323 | 1.00 | 15.19 | A | C |
| ATOM | 899 | CB | ASP | A | 119 | −18.176 | −31.444 | −22.475 | 1.00 | 14.84 | A | C |
| ATOM | 900 | CG | ASP | A | 119 | −17.010 | −31.211 | −23.418 | 1.00 | 14.26 | A | C |
| ATOM | 901 | OD1 | ASP | A | 119 | −16.861 | −31.947 | −24.414 | 1.00 | 14.21 | A | O |
| ATOM | 902 | OD2 | ASP | A | 119 | −16.275 | −30.231 | −23.168 | 1.00 | 13.01 | A | O |
| ATOM | 903 | C | ASP | A | 119 | −19.192 | −33.458 | −23.599 | 1.00 | 16.45 | A | C |
| ATOM | 904 | O | ASP | A | 119 | −20.148 | −32.880 | −24.119 | 1.00 | 16.55 | A | O |
| ATOM | 905 | N | GLY | A | 120 | −18.645 | −34.573 | −24.091 | 1.00 | 18.15 | A | N |
| ATOM | 906 | CA | GLY | A | 120 | −19.134 | −35.212 | −25.316 | 1.00 | 18.51 | A | C |
| ATOM | 907 | C | GLY | A | 120 | −18.466 | −34.701 | −26.573 | 1.00 | 19.67 | A | C |
| ATOM | 908 | O | GLY | A | 120 | −18.547 | −35.358 | −27.603 | 1.00 | 21.88 | A | O |
| ATOM | 909 | N | VAL | A | 121 | −17.798 | −33.549 | −26.504 | 1.00 | 19.75 | A | N |
| ATOM | 910 | CA | VAL | A | 121 | −17.223 | −32.918 | −27.683 | 1.00 | 20.14 | A | C |
| ATOM | 911 | CB | VAL | A | 121 | −17.736 | −31.456 | −27.839 | 1.00 | 20.88 | A | C |
| ATOM | 912 | CG1 | VAL | A | 121 | −16.946 | −30.680 | −28.902 | 1.00 | 21.41 | A | C |
| ATOM | 913 | CG2 | VAL | A | 121 | −19.228 | −31.416 | −28.157 | 1.00 | 20.58 | A | C |
| ATOM | 914 | C | VAL | A | 121 | −15.679 | −32.991 | −27.596 | 1.00 | 20.30 | A | C |
| ATOM | 915 | O | VAL | A | 121 | −15.035 | −33.586 | −28.457 | 1.00 | 19.79 | A | O |
| ATOM | 916 | N | THR | A | 122 | −15.090 | −32.404 | −26.555 | 1.00 | 20.14 | A | N |
| ATOM | 917 | CA | THR | A | 122 | −13.635 | −32.494 | −26.351 | 1.00 | 19.96 | A | C |
| ATOM | 918 | CB | THR | A | 122 | −13.061 | −31.238 | −25.637 | 1.00 | 21.45 | A | C |
| ATOM | 919 | OG1 | THR | A | 122 | −13.999 | −30.796 | −24.662 | 1.00 | 24.36 | A | O |
| ATOM | 920 | CG2 | THR | A | 122 | −12.845 | −30.074 | −26.636 | 1.00 | 21.78 | A | C |
| ATOM | 921 | C | THR | A | 122 | −13.191 | −33.764 | −25.610 | 1.00 | 17.15 | A | C |
| ATOM | 922 | O | THR | A | 122 | −12.032 | −34.113 | −25.706 | 1.00 | 15.97 | A | O |
| ATOM | 923 | N | PHE | A | 123 | −14.103 | −34.428 | −24.892 | 1.00 | 15.54 | A | N |
| ATOM | 924 | CA | PHE | A | 123 | −13.837 | −35.724 | −24.212 | 1.00 | 14.59 | A | C |
| ATOM | 925 | CB | PHE | A | 123 | −13.458 | −35.540 | −22.736 | 1.00 | 14.75 | A | C |
| ATOM | 926 | CG | PHE | A | 123 | −12.146 | −34.912 | −22.502 | 1.00 | 14.63 | A | C |
| ATOM | 927 | CD1 | PHE | A | 123 | −11.007 | −35.663 | −22.517 | 1.00 | 15.07 | A | C |
| ATOM | 928 | CE1 | PHE | A | 123 | −9.772 | −35.088 | −22.268 | 1.00 | 15.58 | A | C |
| ATOM | 929 | CZ | PHE | A | 123 | −9.678 | −33.744 | −21.996 | 1.00 | 15.40 | A | C |
| ATOM | 930 | CE2 | PHE | A | 123 | −10.830 | −32.989 | −21.968 | 1.00 | 15.55 | A | C |
| ATOM | 931 | CD2 | PHE | A | 123 | −12.056 | −33.583 | −22.207 | 1.00 | 15.01 | A | C |
| ATOM | 932 | C | PHE | A | 123 | −15.054 | −36.631 | −24.117 | 1.00 | 13.43 | A | C |
| ATOM | 933 | O | PHE | A | 123 | −16.159 | −36.177 | −24.004 | 1.00 | 13.18 | A | O |
| ATOM | 934 | N | ILE | A | 124 | −14.808 | −37.916 | −24.031 | 1.00 | 13.11 | A | N |
| ATOM | 935 | CA | ILE | A | 124 | −15.829 | −38.898 | −23.717 | 1.00 | 12.99 | A | C |
| ATOM | 936 | CB | ILE | A | 124 | −15.856 | −40.029 | −24.779 | 1.00 | 13.41 | A | C |
| ATOM | 937 | CG1 | ILE | A | 124 | −14.577 | −40.925 | −24.768 | 1.00 | 13.15 | A | C |
| ATOM | 938 | CD1 | ILE | A | 124 | −14.749 | −42.242 | −25.530 | 1.00 | 12.52 | A | C |
| ATOM | 939 | CG2 | ILE | A | 124 | −16.078 | −39.429 | −26.161 | 1.00 | 13.31 | A | C |
| ATOM | 940 | C | ILE | A | 124 | −15.517 | −39.451 | −22.339 | 1.00 | 12.74 | A | C |
| ATOM | 941 | O | ILE | A | 124 | −14.551 | −39.000 | −21.705 | 1.00 | 13.23 | A | O |
| ATOM | 942 | N | PRO | A | 125 | −16.331 | −40.396 | −21.844 | 1.00 | 12.24 | A | N |
| ATOM | 943 | CA | PRO | A | 125 | −16.094 | −40.854 | −20.471 | 1.00 | 12.10 | A | C |
| ATOM | 944 | CB | PRO | A | 125 | −17.132 | −41.978 | −20.285 | 1.00 | 12.45 | A | C |
| ATOM | 945 | CG | PRO | A | 125 | −18.265 | −41.554 | −21.152 | 1.00 | 12.47 | A | C |
| ATOM | 946 | CD | PRO | A | 125 | −17.607 | −40.918 | −22.360 | 1.00 | 12.48 | A | C |
| ATOM | 947 | C | PRO | A | 125 | −14.674 | −41.348 | −20.232 | 1.00 | 11.45 | A | C |
| ATOM | 948 | O | PRO | A | 125 | −14.164 | −42.208 | −20.969 | 1.00 | 10.89 | A | O |
| ATOM | 949 | N | PHE | A | 126 | −14.071 | −40.769 | −19.199 | 1.00 | 10.85 | A | N |
| ATOM | 950 | CA | PHE | A | 126 | −12.645 | −40.875 | −18.936 | 1.00 | 10.71 | A | C |
| ATOM | 951 | CB | PHE | A | 126 | −12.247 | −39.967 | −17.783 | 1.00 | 10.54 | A | C |
| ATOM | 952 | CG | PHE | A | 126 | −12.017 | −38.547 | −18.201 | 1.00 | 10.47 | A | C |
| ATOM | 953 | CD1 | PHE | A | 126 | −13.050 | −37.780 | −18.712 | 1.00 | 10.57 | A | C |
| ATOM | 954 | CE1 | PHE | A | 126 | −12.828 | −36.463 | −19.123 | 1.00 | 10.33 | A | C |
| ATOM | 955 | CZ | PHE | A | 126 | −11.574 | −35.925 | −19.018 | 1.00 | 10.16 | A | C |
| ATOM | 956 | CE2 | PHE | A | 126 | −10.544 | −36.690 | −18.518 | 1.00 | 10.11 | A | C |
| ATOM | 957 | CD2 | PHE | A | 126 | −10.760 | −37.981 | −18.116 | 1.00 | 10.22 | A | C |
| ATOM | 958 | C | PHE | A | 126 | −12.090 | −42.276 | −18.691 | 1.00 | 10.71 | A | C |
| ATOM | 959 | O | PHE | A | 126 | −10.893 | −42.501 | −18.969 | 1.00 | 10.90 | A | O |
| ATOM | 960 | N | SER | A | 127 | −12.930 | −43.220 | −18.246 | 1.00 | 10.31 | A | N |
| ATOM | 961 | CA | SER | A | 127 | −12.456 | −44.588 | −18.018 | 1.00 | 10.08 | A | C |
| ATOM | 962 | CB | SER | A | 127 | −13.430 | −45.374 | −17.180 | 1.00 | 10.12 | A | C |
| ATOM | 963 | OG | SER | A | 127 | −14.738 | −45.075 | −17.550 | 1.00 | 10.16 | A | O |
| ATOM | 964 | C | SER | A | 127 | −12.147 | −45.358 | −19.281 | 1.00 | 9.98 | A | C |
| ATOM | 965 | O | SER | A | 127 | −11.453 | −46.356 | −19.242 | 1.00 | 10.28 | A | O |
| ATOM | 966 | N | ALA | A | 128 | −12.598 | −44.864 | −20.412 | 1.00 | 10.04 | A | N |
| ATOM | 967 | CA | ALA | A | 128 | −12.371 | −45.532 | −21.693 | 1.00 | 10.10 | A | C |
| ATOM | 968 | CB | ALA | A | 128 | −13.180 | −44.831 | −22.782 | 1.00 | 10.16 | A | C |
| ATOM | 969 | C | ALA | A | 128 | −10.899 | −45.715 | −22.144 | 1.00 | 10.16 | A | C |
| ATOM | 970 | O | ALA | A | 128 | −10.633 | −46.559 | −23.017 | 1.00 | 10.10 | A | O |
| ATOM | 971 | N | ASP | A | 129 | −9.957 | −44.956 | −21.580 | 1.00 | 10.09 | A | N |
| ATOM | 972 | CA | ASP | A | 129 | −8.545 | −45.186 | −21.882 | 1.00 | 10.27 | A | C |
| ATOM | 973 | CB | ASP | A | 129 | −7.842 | −43.893 | −22.314 | 1.00 | 10.08 | A | C |
| ATOM | 974 | CG | ASP | A | 129 | −6.541 | −44.156 | −23.060 | 1.00 | 10.15 | A | C |
| ATOM | 975 | OD1 | ASP | A | 129 | −5.845 | −45.112 | −22.681 | 1.00 | 10.22 | A | O |
| ATOM | 976 | OD2 | ASP | A | 129 | −6.191 | −43.422 | −24.030 | 1.00 | 10.06 | A | O |
| ATOM | 977 | C | ASP | A | 129 | −7.836 | −45.809 | −20.686 | 1.00 | 10.67 | A | C |
| ATOM | 978 | O | ASP | A | 129 | −7.789 | −45.228 | −19.607 | 1.00 | 11.36 | A | O |
| ATOM | 979 | N | PRO | A | 130 | −7.247 | −46.992 | −20.866 | 1.00 | 11.05 | A | N |
| ATOM | 980 | CA | PRO | A | 130 | −6.480 | −47.543 | −19.758 | 1.00 | 11.13 | A | C |
| ATOM | 981 | CB | PRO | A | 130 | −5.810 | −48.755 | −20.375 | 1.00 | 11.09 | A | C |
| ATOM | 982 | CG | PRO | A | 130 | −6.739 | −49.200 | −21.455 | 1.00 | 11.13 | A | C |
| ATOM | 983 | CD | PRO | A | 130 | −7.447 | −47.969 | −21.952 | 1.00 | 11.07 | A | C |
| ATOM | 984 | C | PRO | A | 130 | −5.425 | −46.594 | −19.201 | 1.00 | 11.90 | A | C |
| ATOM | 985 | O | PRO | A | 130 | −5.090 | −46.672 | −18.021 | 1.00 | 11.81 | A | O |
| ATOM | 986 | N | ASP | A | 131 | −4.871 | −45.691 | −20.012 | 1.00 | 12.81 | A | N |
| ATOM | 987 | CA | ASP | A | 131 | −3.767 | −44.936 | −19.473 | 1.00 | 13.21 | A | C |
| ATOM | 988 | CB | ASP | A | 131 | −2.861 | −44.298 | −20.538 | 1.00 | 12.98 | A | C |
| ATOM | 989 | CG | ASP | A | 131 | −3.566 | −43.310 | −21.446 | 1.00 | 13.07 | A | C |
| ATOM | 990 | OD1 | ASP | A | 131 | −4.133 | −42.275 | −21.008 | 1.00 | 13.36 | A | O |
| ATOM | 991 | OD2 | ASP | A | 131 | −3.468 | −43.524 | −22.667 | 1.00 | 13.50 | A | O |
| ATOM | 992 | C | ASP | A | 131 | −4.291 | −43.959 | −18.422 | 1.00 | 14.31 | A | C |
| ATOM | 993 | O | ASP | A | 131 | −3.505 | −43.461 | −17.584 | 1.00 | 14.98 | A | O |
| ATOM | 994 | N | VAL | A | 132 | −5.607 | −43.685 | −18.438 | 1.00 | 14.33 | A | N |
| ATOM | 995 | CA | VAL | A | 132 | −6.153 | −42.702 | −17.498 | 1.00 | 14.09 | A | C |
| ATOM | 996 | CB | VAL | A | 132 | −7.528 | −42.180 | −17.937 | 1.00 | 14.79 | A | C |
| ATOM | 997 | CG1 | VAL | A | 132 | −8.084 | −41.168 | −16.922 | 1.00 | 14.72 | A | C |
| ATOM | 998 | CG2 | VAL | A | 132 | −7.420 | −41.532 | −19.314 | 1.00 | 15.01 | A | C |
| ATOM | 999 | C | VAL | A | 132 | −6.253 | −43.352 | −16.140 | 1.00 | 13.25 | A | C |
| ATOM | 1000 | O | VAL | A | 132 | −5.903 | −42.755 | −15.150 | 1.00 | 12.79 | A | O |
| ATOM | 1001 | N | ILE | A | 133 | −6.691 | −44.603 | −16.121 | 1.00 | 12.74 | A | N |
| ATOM | 1002 | CA | ILE | A | 133 | −6.835 | −45.351 | −14.883 | 1.00 | 12.12 | A | C |
| ATOM | 1003 | CB | ILE | A | 133 | −7.565 | −46.703 | −15.122 | 1.00 | 12.05 | A | C |
| ATOM | 1004 | CG1 | ILE | A | 133 | −9.069 | −46.437 | −15.301 | 1.00 | 12.13 | A | C |
| ATOM | 1005 | CD1 | ILE | A | 133 | −9.507 | −46.212 | −16.734 | 1.00 | 12.16 | A | C |
| ATOM | 1006 | CG2 | ILE | A | 133 | −7.403 | −47.665 | −13.948 | 1.00 | 12.02 | A | C |
| ATOM | 1007 | C | ILE | A | 133 | −5.476 | −45.527 | −14.246 | 1.00 | 11.61 | A | C |
| ATOM | 1008 | O | ILE | A | 133 | −5.292 | −45.217 | −13.067 | 1.00 | 11.61 | A | O |
| ATOM | 1009 | N | GLY | A | 134 | −4.515 | −45.989 | −15.029 | 1.00 | 11.10 | A | N |
| ATOM | 1010 | CA | GLY | A | 134 | −3.132 | −46.092 | −14.555 | 1.00 | 10.85 | A | C |
| ATOM | 1011 | C | GLY | A | 134 | −2.611 | −44.784 | −13.982 | 1.00 | 10.75 | A | C |
| ATOM | 1012 | O | GLY | A | 134 | −1.919 | −44.773 | −12.946 | 1.00 | 11.06 | A | O |
| ATOM | 1013 | N | HIS | A | 135 | −2.962 | −43.675 | −14.631 | 1.00 | 10.39 | A | N |
| ATOM | 1014 | CA | HIS | A | 135 | −2.510 | −42.340 | −14.219 | 1.00 | 10.07 | A | C |
| ATOM | 1015 | CB | HIS | A | 135 | −2.987 | −41.329 | −15.262 | 1.00 | 9.93 | A | C |
| ATOM | 1016 | CG | HIS | A | 135 | −2.513 | −39.933 | −15.049 | 1.00 | 9.63 | A | C |
| ATOM | 1017 | ND1 | HIS | A | 135 | −1.472 | −39.389 | −15.765 | 1.00 | 9.67 | A | N |
| ATOM | 1018 | CE1 | HIS | A | 135 | −1.291 | −38.133 | −15.392 | 1.00 | 9.59 | A | C |
| ATOM | 1019 | NE2 | HIS | A | 135 | −2.181 | −37.845 | −14.459 | 1.00 | 9.71 | A | N |
| ATOM | 1020 | CD2 | HIS | A | 135 | −2.964 | −38.953 | −14.237 | 1.00 | 9.62 | A | C |
| ATOM | 1021 | C | HIS | A | 135 | −3.024 | −41.996 | −12.813 | 1.00 | 10.10 | A | C |
| ATOM | 1022 | O | HIS | A | 135 | −2.251 | −41.630 | −11.902 | 1.00 | 10.09 | A | O |
| ATOM | 1023 | N | GLU | A | 136 | −4.326 | −42.136 | −12.624 | 1.00 | 10.10 | A | N |
| ATOM | 1024 | CA | GLU | A | 136 | −4.951 | −41.752 | −11.347 | 1.00 | 10.17 | A | C |
| ATOM | 1025 | CB | GLU | A | 136 | −6.475 | −41.654 | −11.514 | 1.00 | 10.47 | A | C |
| ATOM | 1026 | CG | GLU | A | 136 | −6.894 | −40.641 | −12.554 | 1.00 | 10.55 | A | C |
| ATOM | 1027 | CD | GLU | A | 136 | −6.148 | −39.345 | −12.359 | 1.00 | 11.18 | A | C |
| ATOM | 1028 | OE1 | GLU | A | 136 | −5.972 | −38.931 | −11.195 | 1.00 | 11.67 | A | O |
| ATOM | 1029 | OE2 | GLU | A | 136 | −5.723 | −38.736 | −13.351 | 1.00 | 11.52 | A | O |
| ATOM | 1030 | C | GLU | A | 136 | −4.592 | −42.714 | −10.224 | 1.00 | 9.84 | A | C |
| ATOM | 1031 | O | GLU | A | 136 | −4.247 | −42.287 | −9.155 | 1.00 | 9.75 | A | O |
| ATOM | 1032 | N | LEU | A | 137 | −4.638 | −44.006 | −10.482 | 1.00 | 9.71 | A | N |
| ATOM | 1033 | CA | LEU | A | 137 | −4.113 | −44.998 | −9.549 | 1.00 | 10.13 | A | C |
| ATOM | 1034 | CB | LEU | A | 137 | −4.199 | −46.328 | −10.278 | 1.00 | 10.76 | A | C |
| ATOM | 1035 | CG | LEU | A | 137 | −4.003 | −47.670 | −9.581 | 1.00 | 11.56 | A | C |
| ATOM | 1036 | CD1 | LEU | A | 137 | −2.714 | −47.741 | −8.743 | 1.00 | 11.82 | A | C |
| ATOM | 1037 | CD2 | LEU | A | 137 | −5.230 | −48.010 | −8.760 | 1.00 | 11.81 | A | C |
| ATOM | 1038 | C | LEU | A | 137 | −2.644 | −44.669 | −9.113 | 1.00 | 9.92 | A | C |
| ATOM | 1039 | O | LEU | A | 137 | −2.286 | −44.625 | −7.953 | 1.00 | 9.14 | A | O |
| ATOM | 1040 | N | THR | A | 138 | −1.796 | −44.397 | −10.086 | 1.00 | 10.20 | A | N |
| ATOM | 1041 | CA | THR | A | 138 | −0.425 | −44.011 | −9.817 | 1.00 | 10.26 | A | C |
| ATOM | 1042 | CB | THR | A | 138 | 0.361 | −43.954 | −11.146 | 1.00 | 10.02 | A | C |
| ATOM | 1043 | OG1 | THR | A | 138 | 0.355 | −45.258 | −11.754 | 1.00 | 9.32 | A | O |
| ATOM | 1044 | CG2 | THR | A | 138 | 1.785 | −43.463 | −10.929 | 1.00 | 9.83 | A | C |
| ATOM | 1045 | C | THR | A | 138 | −0.303 | −42.695 | −8.973 | 1.00 | 10.57 | A | C |
| ATOM | 1046 | O | THR | A | 138 | 0.662 | −42.538 | −8.183 | 1.00 | 10.60 | A | O |
| ATOM | 1047 | N | HIS | A | 139 | −1.263 | −41.771 | −9.095 | 1.00 | 10.54 | A | N |
| ATOM | 1048 | CA | HIS | A | 139 | −1.272 | −40.620 | −8.177 | 1.00 | 10.42 | A | C |
| ATOM | 1049 | CB | HIS | A | 139 | −2.468 | −39.695 | −8.370 | 1.00 | 10.27 | A | C |
| ATOM | 1050 | CG | HIS | A | 139 | −2.317 | −38.717 | −9.486 | 1.00 | 10.44 | A | C |
| ATOM | 1051 | ND1 | HIS | A | 139 | −1.094 | −38.259 | −9.918 | 1.00 | 10.59 | A | N |
| ATOM | 1052 | CE1 | HIS | A | 139 | −1.265 | −37.406 | −10.914 | 1.00 | 10.87 | A | C |
| ATOM | 1053 | NE2 | HIS | A | 139 | −2.563 | −37.285 | −11.142 | 1.00 | 11.18 | A | N |
| ATOM | 1054 | CD2 | HIS | A | 139 | −3.241 | −38.089 | −10.249 | 1.00 | 10.94 | A | C |
| ATOM | 1055 | C | HIS | A | 139 | −1.245 | −41.129 | −6.747 | 1.00 | 10.73 | A | C |
| ATOM | 1056 | O | HIS | A | 139 | −0.491 | −40.621 | −5.920 | 1.00 | 10.94 | A | O |
| ATOM | 1057 | N | GLY | A | 140 | −2.052 | −42.151 | −6.466 | 1.00 | 11.04 | A | N |
| ATOM | 1058 | CA | GLY | A | 140 | −2.056 | −42.807 | −5.160 | 1.00 | 10.90 | A | C |
| ATOM | 1059 | C | GLY | A | 140 | −0.692 | −43.407 | −4.803 | 1.00 | 10.98 | A | C |
| ATOM | 1060 | O | GLY | A | 140 | −0.171 | −43.198 | −3.693 | 1.00 | 11.37 | A | O |
| ATOM | 1061 | N | VAL | A | 141 | −0.086 | −44.150 | −5.726 | 1.00 | 10.36 | A | N |
| ATOM | 1062 | CA | VAL | A | 141 | 1.177 | −44.811 | −5.416 | 1.00 | 9.75 | A | C |
| ATOM | 1063 | CB | VAL | A | 141 | 1.682 | −45.578 | −6.619 | 1.00 | 9.53 | A | C |
| ATOM | 1064 | CG1 | VAL | A | 141 | 3.058 | −46.151 | −6.348 | 1.00 | 9.58 | A | C |
| ATOM | 1065 | CG2 | VAL | A | 141 | 0.705 | −46.676 | −6.968 | 1.00 | 9.43 | A | C |
| ATOM | 1066 | C | VAL | A | 141 | 2.215 | −43.784 | −5.013 | 1.00 | 9.84 | A | C |
| ATOM | 1067 | O | VAL | A | 141 | 2.931 | −43.938 | −4.031 | 1.00 | 9.44 | A | O |
| ATOM | 1068 | N | THR | A | 142 | 2.255 | −42.703 | −5.779 | 1.00 | 10.29 | A | N |
| ATOM | 1069 | CA | THR | A | 142 | 3.151 | −41.590 | −5.513 | 1.00 | 10.26 | A | C |
| ATOM | 1070 | CB | THR | A | 142 | 3.000 | −40.503 | −6.577 | 1.00 | 10.08 | A | C |
| ATOM | 1071 | OG1 | THR | A | 142 | 3.358 | −41.026 | −7.880 | 1.00 | 9.80 | A | O |
| ATOM | 1072 | CG2 | THR | A | 142 | 3.871 | −39.305 | −6.210 | 1.00 | 10.03 | A | C |
| ATOM | 1073 | C | THR | A | 142 | 2.904 | −40.965 | −4.158 | 1.00 | 10.52 | A | C |
| ATOM | 1074 | O | THR | A | 142 | 3.843 | −40.718 | −3.399 | 1.00 | 10.56 | A | O |
| ATOM | 1075 | N | GLU | A | 143 | 1.650 | −40.695 | −3.841 | 1.00 | 10.94 | A | N |
| ATOM | 1076 | CA | GLU | A | 143 | 1.352 | −40.031 | −2.570 | 1.00 | 11.25 | A | C |
| ATOM | 1077 | CB | GLU | A | 143 | −0.118 | −39.677 | −2.462 | 1.00 | 11.65 | A | C |
| ATOM | 1078 | CG | GLU | A | 143 | −0.691 | −39.862 | −1.082 | 1.00 | 12.22 | A | C |
| ATOM | 1079 | CD | GLU | A | 143 | −1.934 | −39.052 | −0.832 | 1.00 | 12.99 | A | C |
| ATOM | 1080 | OE1 | GLU | A | 143 | −2.020 | −38.543 | 0.317 | 1.00 | 12.86 | A | O |
| ATOM | 1081 | OE2 | GLU | A | 143 | −2.804 | −38.922 | −1.764 | 1.00 | 13.79 | A | O |
| ATOM | 1082 | C | GLU | A | 143 | 1.782 | −40.903 | −1.415 | 1.00 | 11.45 | A | C |
| ATOM | 1083 | O | GLU | A | 143 | 2.210 | −40.390 | −0.398 | 1.00 | 11.64 | A | O |
| ATOM | 1084 | N | HIS | A | 144 | 1.680 | −42.218 | −1.589 | 1.00 | 11.88 | A | N |
| ATOM | 1085 | CA | HIS | A | 144 | 2.095 | −43.209 | −0.589 | 1.00 | 12.29 | A | C |
| ATOM | 1086 | CB | HIS | A | 144 | 1.247 | −44.484 | −0.746 | 1.00 | 13.27 | A | C |
| ATOM | 1087 | CG | HIS | A | 144 | −0.045 | −44.440 | −0.009 | 1.00 | 14.23 | A | C |
| ATOM | 1088 | ND1 | HIS | A | 144 | −1.080 | −43.600 | −0.368 | 1.00 | 15.20 | A | N |
| ATOM | 1089 | CE1 | HIS | A | 144 | −2.097 | −43.770 | 0.457 | 1.00 | 15.20 | A | C |
| ATOM | 1090 | NE2 | HIS | A | 144 | −1.749 | −44.684 | 1.343 | 1.00 | 15.38 | A | N |
| ATOM | 1091 | CD2 | HIS | A | 144 | −0.467 | −45.116 | 1.079 | 1.00 | 14.62 | A | C |
| ATOM | 1092 | C | HIS | A | 144 | 3.586 | −43.619 | −0.631 | 1.00 | 11.95 | A | C |
| ATOM | 1093 | O | HIS | A | 144 | 4.007 | −44.456 | 0.135 | 1.00 | 11.54 | A | O |
| ATOM | 1094 | N | THR | A | 145 | 4.383 | −43.055 | −1.532 | 1.00 | 11.84 | A | N |
| ATOM | 1095 | CA | THR | A | 145 | 5.798 | −43.407 | −1.620 | 1.00 | 11.38 | A | C |
| ATOM | 1096 | CB | THR | A | 145 | 6.062 | −44.234 | −2.900 | 1.00 | 11.59 | A | C |
| ATOM | 1097 | OG1 | THR | A | 145 | 5.547 | −43.557 | −4.068 | 1.00 | 11.59 | A | O |
| ATOM | 1098 | CG2 | THR | A | 145 | 5.398 | −45.593 | −2.785 | 1.00 | 11.38 | A | C |
| ATOM | 1099 | C | THR | A | 145 | 6.655 | −42.135 | −1.532 | 1.00 | 11.05 | A | C |
| ATOM | 1100 | O | THR | A | 145 | 6.939 | −41.644 | −0.443 | 1.00 | 11.35 | A | O |
| ATOM | 1101 | N | ALA | A | 146 | 7.038 | −41.572 | −2.667 | 1.00 | 10.67 | A | N |
| ATOM | 1102 | CA | ALA | A | 146 | 7.849 | −40.366 | −2.692 | 1.00 | 10.41 | A | C |
| ATOM | 1103 | CB | ALA | A | 146 | 8.226 | −40.046 | −4.120 | 1.00 | 10.53 | A | C |
| ATOM | 1104 | C | ALA | A | 146 | 7.137 | −39.167 | −2.080 | 1.00 | 10.31 | A | C |
| ATOM | 1105 | O | ALA | A | 146 | 7.769 | −38.361 | −1.394 | 1.00 | 9.96 | A | O |
| ATOM | 1106 | N | GLY | A | 147 | 5.827 | −39.051 | −2.349 | 1.00 | 10.33 | A | N |
| ATOM | 1107 | CA | GLY | A | 147 | 5.001 | −37.925 | −1.877 | 1.00 | 10.08 | A | C |
| ATOM | 1108 | C | GLY | A | 147 | 5.238 | −36.619 | −2.623 | 1.00 | 10.00 | A | C |
| ATOM | 1109 | O | GLY | A | 147 | 4.962 | −35.549 | −2.107 | 1.00 | 9.60 | A | O |
| ATOM | 1110 | N | LEU | A | 148 | 5.745 | −36.706 | −3.848 | 1.00 | 10.31 | A | N |
| ATOM | 1111 | CA | LEU | A | 148 | 5.933 | −35.528 | −4.707 | 1.00 | 10.49 | A | C |
| ATOM | 1112 | CB | LEU | A | 148 | 6.018 | −35.939 | −6.165 | 1.00 | 10.25 | A | C |
| ATOM | 1113 | CG | LEU | A | 148 | 7.210 | −36.812 | −6.508 | 1.00 | 10.29 | A | C |
| ATOM | 1114 | CD1 | LEU | A | 148 | 6.994 | −37.473 | −7.848 | 1.00 | 10.47 | A | C |
| ATOM | 1115 | CD2 | LEU | A | 148 | 8.476 | −36.004 | −6.551 | 1.00 | 10.32 | A | C |
| ATOM | 1116 | C | LEU | A | 148 | 4.772 | −34.565 | −4.557 | 1.00 | 10.94 | A | C |
| ATOM | 1117 | O | LEU | A | 148 | 3.636 | −34.881 | −4.915 | 1.00 | 10.71 | A | O |
| ATOM | 1118 | N | GLU | A | 149 | 5.066 | −33.387 | −4.026 | 1.00 | 11.38 | A | N |
| ATOM | 1119 | CA | GLU | A | 149 | 4.042 | −32.384 | −3.824 | 1.00 | 11.72 | A | C |
| ATOM | 1120 | CB | GLU | A | 149 | 4.614 | −31.224 | −3.046 | 1.00 | 12.01 | A | C |
| ATOM | 1121 | CG | GLU | A | 149 | 4.981 | −31.624 | −1.637 | 1.00 | 12.44 | A | C |
| ATOM | 1122 | CD | GLU | A | 149 | 5.857 | −30.602 | −0.954 | 1.00 | 12.98 | A | C |
| ATOM | 1123 | OE1 | GLU | A | 149 | 6.421 | −29.712 | −1.679 | 1.00 | 13.15 | A | O |
| ATOM | 1124 | OE2 | GLU | A | 149 | 5.975 | −30.714 | 0.305 | 1.00 | 12.81 | A | O |
| ATOM | 1125 | C | GLU | A | 149 | 3.503 | −31.865 | −5.139 | 1.00 | 11.83 | A | C |
| ATOM | 1126 | O | GLU | A | 149 | 4.282 | −31.557 | −6.060 | 1.00 | 11.79 | A | O |
| ATOM | 1127 | N | TYR | A | 150 | 2.178 | −31.724 | −5.199 | 1.00 | 11.76 | A | N |
| ATOM | 1128 | CA | TYR | A | 150 | 1.503 | −31.382 | −6.438 | 1.00 | 11.73 | A | C |
| ATOM | 1129 | CB | TYR | A | 150 | 0.047 | −31.854 | −6.381 | 1.00 | 11.48 | A | C |
| ATOM | 1130 | CG | TYR | A | 150 | −0.495 | −32.275 | −7.722 | 1.00 | 11.34 | A | C |
| ATOM | 1131 | CD1 | TYR | A | 150 | −0.009 | −33.409 | −8.366 | 1.00 | 11.48 | A | C |
| ATOM | 1132 | CE1 | TYR | A | 150 | −0.488 | −33.787 | −9.606 | 1.00 | 11.42 | A | C |
| ATOM | 1133 | CZ | TYR | A | 150 | −1.466 | −33.024 | −10.215 | 1.00 | 11.47 | A | C |
| ATOM | 1134 | OH | TYR | A | 150 | −1.983 | −33.385 | −11.434 | 1.00 | 11.35 | A | O |
| ATOM | 1135 | CE2 | TYR | A | 150 | −1.948 | −31.898 | −9.591 | 1.00 | 11.34 | A | C |
| ATOM | 1136 | CD2 | TYR | A | 150 | −1.465 | −31.540 | −8.353 | 1.00 | 11.15 | A | C |
| ATOM | 1137 | C | TYR | A | 150 | 1.594 | −29.893 | −6.778 | 1.00 | 12.17 | A | C |
| ATOM | 1138 | O | TYR | A | 150 | 0.579 | −29.194 | −6.852 | 1.00 | 11.75 | A | O |
| ATOM | 1139 | N | TYR | A | 151 | 2.820 | −29.409 | −7.010 | 1.00 | 13.06 | A | N |
| ATOM | 1140 | CA | TYR | A | 151 | 3.052 | −27.998 | −7.425 | 1.00 | 13.68 | A | C |
| ATOM | 1141 | CB | TYR | A | 151 | 2.853 | −27.019 | −6.258 | 1.00 | 14.30 | A | C |
| ATOM | 1142 | CG | TYR | A | 151 | 2.377 | −25.641 | −6.701 | 1.00 | 15.46 | A | C |
| ATOM | 1143 | CD1 | TYR | A | 151 | 3.284 | −24.631 | −7.111 | 1.00 | 16.45 | A | C |
| ATOM | 1144 | CE1 | TYR | A | 151 | 2.838 | −23.378 | −7.544 | 1.00 | 16.21 | A | C |
| ATOM | 1145 | CZ | TYR | A | 151 | 1.480 | −23.114 | −7.542 | 1.00 | 16.63 | A | C |
| ATOM | 1146 | OH | TYR | A | 151 | 0.955 | −21.884 | −7.943 | 1.00 | 17.27 | A | O |
| ATOM | 1147 | CE2 | TYR | A | 151 | 0.586 | −24.098 | −7.141 | 1.00 | 16.39 | A | C |
| ATOM | 1148 | CD2 | TYR | A | 151 | 1.036 | −25.341 | −6.732 | 1.00 | 15.76 | A | C |
| ATOM | 1149 | C | TYR | A | 151 | 4.455 | −27.817 | −7.980 | 1.00 | 13.62 | A | C |
| ATOM | 1150 | O | TYR | A | 151 | 5.397 | −28.390 | −7.467 | 1.00 | 14.39 | A | O |
| ATOM | 1151 | N | GLY | A | 152 | 4.614 | −27.011 | −9.019 | 1.00 | 13.51 | A | N |
| ATOM | 1152 | CA | GLY | A | 152 | 5.939 | −26.701 | −9.531 | 1.00 | 13.34 | A | C |
| ATOM | 1153 | C | GLY | A | 152 | 6.653 | −27.917 | −10.097 | 1.00 | 13.83 | A | C |
| ATOM | 1154 | O | GLY | A | 152 | 6.032 | −28.807 | −10.695 | 1.00 | 14.49 | A | O |
| ATOM | 1155 | N | GLU | A | 153 | 7.966 | −27.961 | −9.916 | 1.00 | 13.72 | A | N |
| ATOM | 1156 | CA | GLU | A | 153 | 8.749 | −29.047 | −10.468 | 1.00 | 13.56 | A | C |
| ATOM | 1157 | CB | GLU | A | 153 | 10.238 | −28.725 | −10.362 | 1.00 | 13.75 | A | C |
| ATOM | 1158 | CG | GLU | A | 153 | 10.664 | −27.555 | −11.240 | 1.00 | 13.88 | A | C |
| ATOM | 1159 | CD | GLU | A | 153 | 12.180 | −27.399 | −11.424 | 1.00 | 14.09 | A | C |
| ATOM | 1160 | OE1 | GLU | A | 153 | 12.942 | −28.380 | −11.352 | 1.00 | 14.77 | A | O |
| ATOM | 1161 | OE2 | GLU | A | 153 | 12.626 | −26.283 | −11.700 | 1.00 | 13.79 | A | O |
| ATOM | 1162 | C | GLU | A | 153 | 8.392 | −30.427 | −9.867 | 1.00 | 13.23 | A | C |
| ATOM | 1163 | O | GLU | A | 153 | 8.317 | −31.407 | −10.607 | 1.00 | 13.02 | A | O |
| ATOM | 1164 | N | SER | A | 154 | 8.152 | −30.502 | −8.556 | 1.00 | 13.06 | A | N |
| ATOM | 1165 | CA | SER | A | 154 | 7.720 | −31.766 | −7.919 | 1.00 | 12.61 | A | C |
| ATOM | 1166 | CB | SER | A | 154 | 7.509 | −31.629 | −6.409 | 1.00 | 12.60 | A | C |
| ATOM | 1167 | OG | SER | A | 154 | 6.935 | −30.397 | −6.002 | 1.00 | 12.59 | A | O |
| ATOM | 1168 | C | SER | A | 154 | 6.438 | −32.211 | −8.546 | 1.00 | 12.47 | A | C |
| ATOM | 1169 | O | SER | A | 154 | 6.205 | −33.388 | −8.804 | 1.00 | 12.67 | A | O |
| ATOM | 1170 | N | GLY | A | 155 | 5.586 | −31.245 | −8.817 | 1.00 | 12.56 | A | N |
| ATOM | 1171 | CA | GLY | A | 155 | 4.310 | −31.540 | −9.447 | 1.00 | 12.03 | A | C |
| ATOM | 1172 | C | GLY | A | 155 | 4.443 | −32.051 | −10.869 | 1.00 | 11.39 | A | C |
| ATOM | 1173 | O | GLY | A | 155 | 3.646 | −32.860 | −11.295 | 1.00 | 11.89 | A | O |
| ATOM | 1174 | N | ALA | A | 156 | 5.413 | −31.568 | −11.633 | 1.00 | 10.56 | A | N |
| ATOM | 1175 | CA | ALA | A | 156 | 5.536 | −32.047 | −13.008 | 1.00 | 9.90 | A | C |
| ATOM | 1176 | CB | ALA | A | 156 | 6.481 | −31.175 | −13.804 | 1.00 | 9.92 | A | C |
| ATOM | 1177 | C | ALA | A | 156 | 6.015 | −33.501 | −13.004 | 1.00 | 9.25 | A | C |
| ATOM | 1178 | O | ALA | A | 156 | 5.502 | −34.330 | −13.728 | 1.00 | 9.03 | A | O |
| ATOM | 1179 | N | LEU | A | 157 | 7.020 | −33.773 | −12.184 | 1.00 | 8.76 | A | N |
| ATOM | 1180 | CA | LEU | A | 157 | 7.532 | −35.104 | −11.970 | 1.00 | 8.36 | A | C |
| ATOM | 1181 | CB | LEU | A | 157 | 8.540 | −35.070 | −10.837 | 1.00 | 8.37 | A | C |
| ATOM | 1182 | CG | LEU | A | 157 | 10.041 | −35.045 | −11.062 | 1.00 | 8.61 | A | C |
| ATOM | 1183 | CD1 | LEU | A | 157 | 10.463 | −34.666 | −12.454 | 1.00 | 8.77 | A | C |
| ATOM | 1184 | CD2 | LEU | A | 157 | 10.705 | −34.119 | −10.052 | 1.00 | 8.91 | A | C |
| ATOM | 1185 | C | LEU | A | 157 | 6.376 | −36.025 | −11.596 | 1.00 | 8.07 | A | C |
| ATOM | 1186 | O | LEU | A | 157 | 6.255 | −37.135 | −12.142 | 1.00 | 7.77 | A | O |
| ATOM | 1187 | N | ASN | A | 158 | 5.538 | −35.544 | −10.669 | 1.00 | 7.73 | A | N |
| ATOM | 1188 | CA | ASN | A | 158 | 4.376 | −36.292 | −10.195 | 1.00 | 7.65 | A | C |
| ATOM | 1189 | CB | ASN | A | 158 | 3.618 | −35.443 | −9.175 | 1.00 | 7.60 | A | C |
| ATOM | 1190 | CG | ASN | A | 158 | 2.454 | −36.174 | −8.544 | 1.00 | 7.45 | A | C |
| ATOM | 1191 | OD1 | ASN | A | 158 | 2.219 | −36.081 | −7.338 | 1.00 | 7.24 | A | O |
| ATOM | 1192 | ND2 | ASN | A | 158 | 1.713 | −36.889 | −9.354 | 1.00 | 7.41 | A | N |
| ATOM | 1193 | C | ASN | A | 158 | 3.454 | −36.711 | −11.351 | 1.00 | 7.65 | A | C |
| ATOM | 1194 | O | ASN | A | 158 | 3.092 | −37.888 | −11.501 | 1.00 | 7.48 | A | O |
| ATOM | 1195 | N | GLU | A | 159 | 3.108 | −35.731 | −12.180 | 1.00 | 7.81 | A | N |
| ATOM | 1196 | CA | GLU | A | 159 | 2.317 | −35.962 | −13.381 | 1.00 | 7.91 | A | C |
| ATOM | 1197 | CB | GLU | A | 159 | 1.910 | −34.630 | −14.000 | 1.00 | 7.94 | A | C |
| ATOM | 1198 | CG | GLU | A | 159 | 0.601 | −34.083 | −13.429 | 1.00 | 8.02 | A | C |
| ATOM | 1199 | CD | GLU | A | 159 | −0.576 | −34.968 | −13.795 | 1.00 | 8.26 | A | C |
| ATOM | 1200 | OE1 | GLU | A | 159 | −0.653 | −35.249 | −15.012 | 1.00 | 8.90 | A | O |
| ATOM | 1201 | OE2 | GLU | A | 159 | −1.397 | −35.417 | −12.935 | 1.00 | 8.04 | A | O |
| ATOM | 1202 | C | GLU | A | 159 | 3.072 | −36.857 | −14.356 | 1.00 | 8.02 | A | C |
| ATOM | 1203 | O | GLU | A | 159 | 2.509 | −37.755 | −14.950 | 1.00 | 8.32 | A | O |
| ATOM | 1204 | N | SER | A | 160 | 4.373 | −36.676 | −14.460 | 1.00 | 8.05 | A | N |
| ATOM | 1205 | CA | SER | A | 160 | 5.141 | −37.443 | −15.423 | 1.00 | 7.94 | A | C |
| ATOM | 1206 | CB | SER | A | 160 | 6.597 | −36.950 | −15.477 | 1.00 | 7.76 | A | C |
| ATOM | 1207 | OG | SER | A | 160 | 7.318 | −37.607 | −16.500 | 1.00 | 7.64 | A | O |
| ATOM | 1208 | C | SER | A | 160 | 5.060 | −38.907 | −15.036 | 1.00 | 8.03 | A | C |
| ATOM | 1209 | O | SER | A | 160 | 4.736 | −39.785 | −15.860 | 1.00 | 7.90 | A | O |
| ATOM | 1210 | N | ILE | A | 161 | 5.343 | −39.165 | −13.767 | 1.00 | 8.08 | A | N |
| ATOM | 1211 | CA | ILE | A | 161 | 5.358 | −40.532 | −13.276 | 1.00 | 8.23 | A | C |
| ATOM | 1212 | CB | ILE | A | 161 | 5.615 | −40.592 | −11.763 | 1.00 | 8.23 | A | C |
| ATOM | 1213 | CG1 | ILE | A | 161 | 7.021 | −40.033 | −11.446 | 1.00 | 8.35 | A | C |
| ATOM | 1214 | CD1 | ILE | A | 161 | 8.170 | −41.019 | −11.674 | 1.00 | 8.40 | A | C |
| ATOM | 1215 | CG2 | ILE | A | 161 | 5.459 | −42.016 | −11.243 | 1.00 | 8.11 | A | C |
| ATOM | 1216 | C | ILE | A | 161 | 4.017 | −41.190 | −13.593 | 1.00 | 8.64 | A | C |
| ATOM | 1217 | O | ILE | A | 161 | 3.995 | −42.329 | −14.107 | 1.00 | 8.86 | A | O |
| ATOM | 1218 | N | SER | A | 162 | 2.903 | −40.468 | −13.327 | 1.00 | 8.55 | A | N |
| ATOM | 1219 | CA | SER | A | 162 | 1.563 | −41.009 | −13.583 | 1.00 | 8.24 | A | C |
| ATOM | 1220 | CB | SER | A | 162 | 0.470 | −40.063 | −13.041 | 1.00 | 8.36 | A | C |
| ATOM | 1221 | OG | SER | A | 162 | 0.268 | −40.222 | −11.610 | 1.00 | 8.36 | A | O |
| ATOM | 1222 | C | SER | A | 162 | 1.401 | −41.315 | −15.066 | 1.00 | 7.96 | A | C |
| ATOM | 1223 | O | SER | A | 162 | 0.870 | −42.360 | −15.452 | 1.00 | 7.78 | A | O |
| ATOM | 1224 | N | ASP | A | 163 | 1.900 | −40.410 | −15.891 | 1.00 | 7.89 | A | N |
| ATOM | 1225 | CA | ASP | A | 163 | 1.939 | −40.609 | −17.346 | 1.00 | 8.00 | A | C |
| ATOM | 1226 | CB | ASP | A | 163 | 2.451 | −39.323 | −18.029 | 1.00 | 7.81 | A | C |
| ATOM | 1227 | CG | ASP | A | 163 | 1.371 | −38.310 | −18.242 | 1.00 | 7.61 | A | C |
| ATOM | 1228 | OD1 | ASP | A | 163 | 0.228 | −38.669 | −17.919 | 1.00 | 7.69 | A | O |
| ATOM | 1229 | OD2 | ASP | A | 163 | 1.637 | −37.194 | −18.750 | 1.00 | 7.24 | A | O |
| ATOM | 1230 | C | ASP | A | 163 | 2.842 | −41.791 | −17.764 | 1.00 | 8.39 | A | C |
| ATOM | 1231 | O | ASP | A | 163 | 2.464 | −42.654 | −18.573 | 1.00 | 8.14 | A | O |
| ATOM | 1232 | N | ILE | A | 164 | 4.058 | −41.823 | −17.223 | 1.00 | 8.75 | A | N |
| ATOM | 1233 | CA | ILE | A | 164 | 4.955 | −42.900 | −17.590 | 1.00 | 9.10 | A | C |
| ATOM | 1234 | CB | ILE | A | 164 | 6.308 | −42.744 | −16.902 | 1.00 | 8.62 | A | C |
| ATOM | 1235 | CG1 | ILE | A | 164 | 7.058 | −41.631 | −17.541 | 1.00 | 8.29 | A | C |
| ATOM | 1236 | CD1 | ILE | A | 164 | 7.945 | −40.950 | −16.552 | 1.00 | 8.37 | A | C |
| ATOM | 1237 | CG2 | ILE | A | 164 | 7.109 | −44.018 | −17.007 | 1.00 | 8.59 | A | C |
| ATOM | 1238 | C | ILE | A | 164 | 4.326 | −44.280 | −17.276 | 1.00 | 9.63 | A | C |
| ATOM | 1239 | O | ILE | A | 164 | 4.408 | −45.188 | −18.076 | 1.00 | 9.83 | A | O |
| ATOM | 1240 | N | ILE | A | 165 | 3.688 | −44.427 | −16.125 | 1.00 | 10.31 | A | N |
| ATOM | 1241 | CA | ILE | A | 165 | 3.113 | −45.730 | −15.759 | 1.00 | 11.09 | A | C |
| ATOM | 1242 | CB | ILE | A | 165 | 3.142 | −45.965 | −14.238 | 1.00 | 11.45 | A | C |
| ATOM | 1243 | CG1 | ILE | A | 165 | 4.583 | −46.312 | −13.859 | 1.00 | 11.73 | A | C |
| ATOM | 1244 | CD1 | ILE | A | 165 | 4.846 | −46.351 | −12.376 | 1.00 | 12.06 | A | C |
| ATOM | 1245 | CG2 | ILE | A | 165 | 2.257 | −47.137 | −13.843 | 1.00 | 11.71 | A | C |
| ATOM | 1246 | C | ILE | A | 165 | 1.743 | −45.953 | −16.396 | 1.00 | 11.24 | A | C |
| ATOM | 1247 | O | ILE | A | 165 | 1.519 | −46.993 | −16.983 | 1.00 | 10.71 | A | O |
| ATOM | 1248 | N | GLY | A | 166 | 0.866 | −44.956 | −16.352 | 1.00 | 11.84 | A | N |
| ATOM | 1249 | CA | GLY | A | 166 | −0.347 | −44.989 | −17.161 | 1.00 | 12.20 | A | C |
| ATOM | 1250 | C | GLY | A | 166 | −0.114 | −45.522 | −18.568 | 1.00 | 12.55 | A | C |
| ATOM | 1251 | O | GLY | A | 166 | −0.856 | −46.371 | −19.055 | 1.00 | 13.27 | A | O |
| ATOM | 1252 | N | ASN | A | 167 | 0.936 | −45.042 | −19.219 | 1.00 | 13.01 | A | N |
| ATOM | 1253 | CA | ASN | A | 167 | 1.211 | −45.411 | −20.617 | 1.00 | 13.46 | A | C |
| ATOM | 1254 | CB | ASN | A | 167 | 2.130 | −44.379 | −21.255 | 1.00 | 13.54 | A | C |
| ATOM | 1255 | CG | ASN | A | 167 | 2.598 | −44.799 | −22.620 | 1.00 | 13.80 | A | C |
| ATOM | 1256 | OD1 | ASN | A | 167 | 3.650 | −45.428 | −22.768 | 1.00 | 13.76 | A | O |
| ATOM | 1257 | ND2 | ASN | A | 167 | 1.794 | −44.495 | −23.630 | 1.00 | 13.97 | A | N |
| ATOM | 1258 | C | ASN | A | 167 | 1.831 | −46.800 | −20.769 | 1.00 | 13.70 | A | C |
| ATOM | 1259 | O | ASN | A | 167 | 1.633 | −47.497 | −21.769 | 1.00 | 13.68 | A | O |
| ATOM | 1260 | N | ALA | A | 168 | 2.607 | −47.179 | −19.767 | 1.00 | 14.07 | A | N |
| ATOM | 1261 | CA | ALA | A | 168 | 3.260 | −48.453 | −19.765 | 1.00 | 13.94 | A | C |
| ATOM | 1262 | CB | ALA | A | 168 | 4.247 | −48.531 | −18.618 | 1.00 | 13.53 | A | C |
| ATOM | 1263 | C | ALA | A | 168 | 2.231 | −49.547 | −19.667 | 1.00 | 14.33 | A | C |
| ATOM | 1264 | O | ALA | A | 168 | 2.429 | −50.581 | −20.227 | 1.00 | 13.98 | A | O |
| ATOM | 1265 | N | ILE | A | 169 | 1.131 | −49.324 | −18.960 | 1.00 | 15.94 | A | N |
| ATOM | 1266 | CA | ILE | A | 169 | 0.218 | −50.428 | −18.655 | 1.00 | 18.10 | A | C |
| ATOM | 1267 | CB | ILE | A | 169 | −0.969 | −50.037 | −17.743 | 1.00 | 18.79 | A | C |
| ATOM | 1268 | CG1 | ILE | A | 169 | −0.561 | −50.080 | −16.280 | 1.00 | 19.73 | A | C |
| ATOM | 1269 | CD1 | ILE | A | 169 | −0.048 | −48.784 | −15.730 | 1.00 | 20.46 | A | C |
| ATOM | 1270 | CG2 | ILE | A | 169 | −2.130 | −51.016 | −17.942 | 1.00 | 18.64 | A | C |
| ATOM | 1271 | C | ILE | A | 169 | −0.394 | −50.822 | −19.949 | 1.00 | 19.50 | A | C |
| ATOM | 1272 | O | ILE | A | 169 | −0.420 | −51.982 | −20.301 | 1.00 | 17.56 | A | O |
| ATOM | 1273 | N | ASP | A | 170 | −0.941 | −49.782 | −20.581 | 1.00 | 22.96 | A | N |
| ATOM | 1274 | CA | ASP | A | 170 | −1.495 | −49.783 | −21.922 | 1.00 | 25.80 | A | C |
| ATOM | 1275 | CB | ASP | A | 170 | −1.761 | −48.290 | −22.319 | 1.00 | 28.80 | A | C |
| ATOM | 1276 | CG | ASP | A | 170 | −3.056 | −48.077 | −23.198 | 1.00 | 31.65 | A | C |
| ATOM | 1277 | OD1 | ASP | A | 170 | −3.727 | −49.094 | −23.585 | 1.00 | 32.68 | A | O |
| ATOM | 1278 | OD2 | ASP | A | 170 | −3.385 | −46.874 | −23.485 | 1.00 | 27.68 | A | O |
| ATOM | 1279 | C | ASP | A | 170 | −0.556 | −50.556 | −22.895 | 1.00 | 25.17 | A | C |
| ATOM | 1280 | O | ASP | A | 170 | −0.916 | −51.613 | −23.330 | 1.00 | 26.97 | A | O |
| ATOM | 1281 | N | GLY | A | 171 | 0.654 | −50.077 | −23.186 | 1.00 | 25.32 | A | N |
| ATOM | 1282 | CA | GLY | A | 171 | 1.589 | −50.813 | −24.048 | 1.00 | 25.14 | A | C |
| ATOM | 1283 | C | GLY | A | 171 | 1.799 | −50.195 | −25.428 | 1.00 | 26.60 | A | C |
| ATOM | 1284 | O | GLY | A | 171 | 2.914 | −50.233 | −25.976 | 1.00 | 26.53 | A | O |
| ATOM | 1285 | N | LYS | A | 172 | 0.759 | −49.524 | −25.930 | 1.00 | 26.94 | A | N |
| ATOM | 1286 | CA | LYS | A | 172 | 0.444 | −49.463 | −27.366 | 1.00 | 26.88 | A | C |
| ATOM | 1287 | CB | LYS | A | 172 | −1.051 | −49.132 | −27.537 | 1.00 | 30.72 | A | C |
| ATOM | 1288 | CG | LYS | A | 172 | −2.071 | −50.219 | −27.134 | 1.00 | 33.65 | A | C |
| ATOM | 1289 | CD | LYS | A | 172 | −3.392 | −50.025 | −27.922 | 1.00 | 36.81 | A | C |
| ATOM | 1290 | CE | LYS | A | 172 | −4.669 | −50.070 | −27.073 | 1.00 | 38.06 | A | C |
| ATOM | 1291 | NZ | LYS | A | 172 | −5.038 | −48.814 | −26.339 | 1.00 | 36.90 | A | N |
| ATOM | 1292 | C | LYS | A | 172 | 1.213 | −48.471 | −28.238 | 1.00 | 23.70 | A | C |
| ATOM | 1293 | O | LYS | A | 172 | 1.584 | −48.768 | −29.365 | 1.00 | 23.20 | A | O |
| ATOM | 1294 | N | ASN | A | 173 | 1.390 | −47.273 | −27.716 | 1.00 | 20.08 | A | N |
| ATOM | 1295 | CA | ASN | A | 173 | 1.898 | −46.146 | −28.467 | 1.00 | 17.00 | A | C |
| ATOM | 1296 | CB | ASN | A | 173 | 0.736 | −45.383 | −29.125 | 1.00 | 16.90 | A | C |
| ATOM | 1297 | CG | ASN | A | 173 | −0.422 | −45.079 | −28.148 | 1.00 | 16.63 | A | C |
| ATOM | 1298 | OD1 | ASN | A | 173 | −0.260 | −44.495 | −27.064 | 1.00 | 15.50 | A | O |
| ATOM | 1299 | ND2 | ASN | A | 173 | −1.602 | −45.470 | −28.552 | 1.00 | 16.78 | A | N |
| ATOM | 1300 | C | ASN | A | 173 | 2.538 | −45.275 | −27.429 | 1.00 | 14.76 | A | C |
| ATOM | 1301 | O | ASN | A | 173 | 2.573 | −45.681 | −26.284 | 1.00 | 15.23 | A | O |
| ATOM | 1302 | N | TRP | A | 174 | 2.986 | −44.078 | −27.784 | 1.00 | 12.57 | A | N |
| ATOM | 1303 | CA | TRP | A | 174 | 3.504 | −43.159 | −26.790 | 1.00 | 11.39 | A | C |
| ATOM | 1304 | CB | TRP | A | 174 | 4.895 | −42.700 | −27.180 | 1.00 | 11.51 | A | C |
| ATOM | 1305 | CG | TRP | A | 174 | 5.894 | −43.735 | −27.116 | 1.00 | 11.10 | A | C |
| ATOM | 1306 | CD1 | TRP | A | 174 | 6.445 | −44.349 | −28.137 | 1.00 | 10.94 | A | C |
| ATOM | 1307 | NE1 | TRP | A | 174 | 7.375 | −45.235 | −27.722 | 1.00 | 11.20 | A | N |
| ATOM | 1308 | CE2 | TRP | A | 174 | 7.431 | −45.189 | −26.362 | 1.00 | 11.59 | A | C |
| ATOM | 1309 | CD2 | TRP | A | 174 | 6.499 | −44.247 | −25.953 | 1.00 | 11.43 | A | C |
| ATOM | 1310 | CE3 | TRP | A | 174 | 6.344 | −44.000 | −24.584 | 1.00 | 11.96 | A | C |
| ATOM | 1311 | CZ3 | TRP | A | 174 | 7.125 | −44.722 | −23.675 | 1.00 | 11.80 | A | C |
| ATOM | 1312 | CH2 | TRP | A | 174 | 8.045 | −45.657 | −24.122 | 1.00 | 11.84 | A | C |
| ATOM | 1313 | CZ2 | TRP | A | 174 | 8.216 | −45.910 | −25.461 | 1.00 | 11.89 | A | C |
| ATOM | 1314 | C | TRP | A | 174 | 2.619 | −41.936 | −26.558 | 1.00 | 10.44 | A | C |
| ATOM | 1315 | O | TRP | A | 174 | 3.119 | −40.869 | −26.219 | 1.00 | 9.94 | A | O |
| ATOM | 1316 | N | LEU | A | 175 | 1.309 | −42.121 | −26.673 | 1.00 | 9.73 | A | N |
| ATOM | 1317 | CA | LEU | A | 175 | 0.346 | −41.082 | −26.370 | 1.00 | 9.50 | A | C |
| ATOM | 1318 | CB | LEU | A | 175 | −0.739 | −41.054 | −27.450 | 1.00 | 9.31 | A | C |
| ATOM | 1319 | CG | LEU | A | 175 | −0.164 | −41.226 | −28.865 | 1.00 | 9.05 | A | C |
| ATOM | 1320 | CD1 | LEU | A | 175 | −1.260 | −41.361 | −29.880 | 1.00 | 8.82 | A | C |
| ATOM | 1321 | CD2 | LEU | A | 175 | 0.776 | −40.096 | −29.223 | 1.00 | 8.97 | A | C |
| ATOM | 1322 | C | LEU | A | 175 | −0.308 | −41.248 | −24.998 | 1.00 | 9.46 | A | C |
| ATOM | 1323 | O | LEU | A | 175 | −0.256 | −42.298 | −24.395 | 1.00 | 9.36 | A | O |
| ATOM | 1324 | N | ILE | A | 176 | −0.927 | −40.172 | −24.528 | 1.00 | 9.67 | A | N |
| ATOM | 1325 | CA | ILE | A | 176 | −1.810 | −40.190 | −23.373 | 1.00 | 9.57 | A | C |
| ATOM | 1326 | CB | ILE | A | 176 | −1.257 | −39.265 | −22.274 | 1.00 | 9.83 | A | C |
| ATOM | 1327 | CG1 | ILE | A | 176 | 0.182 | −39.690 | −21.898 | 1.00 | 10.01 | A | C |
| ATOM | 1328 | CD1 | ILE | A | 176 | 0.280 | −41.016 | −21.150 | 1.00 | 9.93 | A | C |
| ATOM | 1329 | CG2 | ILE | A | 176 | −2.184 | −39.218 | −21.049 | 1.00 | 9.82 | A | C |
| ATOM | 1330 | C | ILE | A | 176 | −3.206 | −39.704 | −23.778 | 1.00 | 9.56 | A | C |
| ATOM | 1331 | O | ILE | A | 176 | −3.349 | −38.639 | −24.428 | 1.00 | 8.84 | A | O |
| ATOM | 1332 | N | GLY | A | 177 | −4.220 | −40.486 | −23.367 | 1.00 | 9.68 | A | N |
| ATOM | 1333 | CA | GLY | A | 177 | −5.622 | −40.131 | −23.517 | 1.00 | 9.79 | A | C |
| ATOM | 1334 | C | GLY | A | 177 | −6.120 | −40.170 | −24.946 | 1.00 | 10.29 | A | C |
| ATOM | 1335 | O | GLY | A | 177 | −7.167 | −39.604 | −25.249 | 1.00 | 10.78 | A | O |
| ATOM | 1336 | N | ASP | A | 178 | −5.381 | −40.826 | −25.840 | 1.00 | 10.68 | A | N |
| ATOM | 1337 | CA | ASP | A | 178 | −5.808 | −40.998 | −27.226 | 1.00 | 10.63 | A | C |
| ATOM | 1338 | CB | ASP | A | 178 | −4.848 | −41.873 | −28.063 | 1.00 | 10.79 | A | C |
| ATOM | 1339 | CG | ASP | A | 178 | −4.414 | −43.165 | −27.352 | 1.00 | 11.25 | A | C |
| ATOM | 1340 | OD1 | ASP | A | 178 | −4.028 | −43.125 | −26.137 | 1.00 | 11.72 | A | O |
| ATOM | 1341 | OD2 | ASP | A | 178 | −4.445 | −44.230 | −28.017 | 1.00 | 11.08 | A | O |
| ATOM | 1342 | C | ASP | A | 178 | −7.221 | −41.536 | −27.336 | 1.00 | 10.59 | A | C |
| ATOM | 1343 | O | ASP | A | 178 | −7.904 | −41.156 | −28.272 | 1.00 | 11.28 | A | O |
| ATOM | 1344 | N | LEU | A | 179 | −7.671 | −42.364 | −26.395 | 1.00 | 10.20 | A | N |
| ATOM | 1345 | CA | LEU | A | 179 | −8.985 | −43.023 | −26.512 | 1.00 | 10.39 | A | C |
| ATOM | 1346 | CB | LEU | A | 179 | −8.959 | −44.429 | −25.884 | 1.00 | 10.21 | A | C |
| ATOM | 1347 | CG | LEU | A | 179 | −8.140 | −45.483 | −26.620 | 1.00 | 10.17 | A | C |
| ATOM | 1348 | CD1 | LEU | A | 179 | −8.127 | −46.713 | −25.763 | 1.00 | 10.31 | A | C |
| ATOM | 1349 | CD2 | LEU | A | 179 | −8.653 | −45.828 | −28.003 | 1.00 | 9.96 | A | C |
| ATOM | 1350 | C | LEU | A | 179 | −10.171 | −42.272 | −25.891 | 1.00 | 10.69 | A | C |
| ATOM | 1351 | O | LEU | A | 179 | −11.324 | −42.757 | −25.993 | 1.00 | 10.57 | A | O |
| ATOM | 1352 | N | ILE | A | 180 | −9.884 | −41.142 | −25.240 | 1.00 | 10.58 | A | N |
| ATOM | 1353 | CA | ILE | A | 180 | −10.901 | −40.329 | −24.611 | 1.00 | 10.92 | A | C |
| ATOM | 1354 | CB | ILE | A | 180 | −10.662 | −40.171 | −23.101 | 1.00 | 11.08 | A | C |
| ATOM | 1355 | CG1 | ILE | A | 180 | −9.227 | −39.759 | −22.788 | 1.00 | 10.99 | A | C |
| ATOM | 1356 | CD1 | ILE | A | 180 | −9.107 | −39.174 | −21.404 | 1.00 | 11.00 | A | C |
| ATOM | 1357 | CG2 | ILE | A | 180 | −10.988 | −41.455 | −22.355 | 1.00 | 11.54 | A | C |
| ATOM | 1358 | C | ILE | A | 180 | −11.031 | −38.915 | −25.192 | 1.00 | 11.35 | A | C |
| ATOM | 1359 | O | ILE | A | 180 | −12.085 | −38.286 | −25.012 | 1.00 | 11.56 | A | O |
| ATOM | 1360 | N | TYR | A | 181 | −9.969 | −38.421 | −25.844 | 1.00 | 11.08 | A | N |
| ATOM | 1361 | CA | TYR | A | 181 | −9.888 | −37.060 | −26.366 | 1.00 | 11.01 | A | C |
| ATOM | 1362 | CB | TYR | A | 181 | −8.420 | −36.640 | −26.406 | 1.00 | 10.90 | A | C |
| ATOM | 1363 | CG | TYR | A | 181 | −8.183 | −35.157 | −26.669 | 1.00 | 10.79 | A | C |
| ATOM | 1364 | CD1 | TYR | A | 181 | −8.717 | −34.181 | −25.830 | 1.00 | 10.58 | A | C |
| ATOM | 1365 | CE1 | TYR | A | 181 | −8.480 | −32.830 | −26.058 | 1.00 | 10.73 | A | C |
| ATOM | 1366 | CZ | TYR | A | 181 | −7.665 | −32.423 | −27.122 | 1.00 | 10.52 | A | C |
| ATOM | 1367 | OH | TYR | A | 181 | −7.417 | −31.084 | −27.349 | 1.00 | 9.91 | A | O |
| ATOM | 1368 | CE2 | TYR | A | 181 | −7.122 | −33.375 | −27.956 | 1.00 | 10.67 | A | C |
| ATOM | 1369 | CD2 | TYR | A | 181 | −7.377 | −34.734 | −27.723 | 1.00 | 10.92 | A | C |
| ATOM | 1370 | C | TYR | A | 181 | −10.429 | −36.894 | −27.772 | 1.00 | 11.14 | A | C |
| ATOM | 1371 | O | TYR | A | 181 | −10.050 | −37.616 | −28.648 | 1.00 | 11.55 | A | O |
| ATOM | 1372 | N | THR | A | 182 | −11.277 | −35.901 | −28.001 | 1.00 | 12.04 | A | N |
| ATOM | 1373 | CA | THR | A | 182 | −11.754 | −35.522 | −29.366 | 1.00 | 12.29 | A | C |
| ATOM | 1374 | CB | THR | A | 182 | −10.647 | −34.772 | −30.147 | 1.00 | 12.04 | A | C |
| ATOM | 1375 | OG1 | THR | A | 182 | −9.549 | −35.675 | −30.435 | 1.00 | 11.78 | A | O |
| ATOM | 1376 | CG2 | THR | A | 182 | −10.182 | −33.532 | −29.383 | 1.00 | 11.44 | A | C |
| ATOM | 1377 | C | THR | A | 182 | −12.249 | −36.731 | −30.203 | 1.00 | 12.70 | A | C |
| ATOM | 1378 | O | THR | A | 182 | −11.491 | −37.287 | −31.039 | 1.00 | 12.48 | A | O |
| ATOM | 1379 | N | PRO | A | 183 | −13.497 | −37.159 | −29.955 | 1.00 | 13.03 | A | N |
| ATOM | 1380 | CA | PRO | A | 183 | −14.066 | −38.320 | −30.627 | 1.00 | 13.57 | A | C |
| ATOM | 1381 | CB | PRO | A | 183 | −15.451 | −38.487 | −29.993 | 1.00 | 13.44 | A | C |
| ATOM | 1382 | CG | PRO | A | 183 | −15.611 | −37.419 | −28.985 | 1.00 | 13.35 | A | C |
| ATOM | 1383 | CD | PRO | A | 183 | −14.320 | −36.699 | −28.831 | 1.00 | 13.41 | A | C |
| ATOM | 1384 | C | PRO | A | 183 | −14.199 | −38.181 | −32.135 | 1.00 | 13.75 | A | C |
| ATOM | 1385 | O | PRO | A | 183 | −14.230 | −39.180 | −32.839 | 1.00 | 13.12 | A | O |
| ATOM | 1386 | N | ASN | A | 184 | −14.285 | −36.955 | −32.616 | 1.00 | 14.63 | A | N |
| ATOM | 1387 | CA | ASN | A | 184 | −14.499 | −36.726 | −34.034 | 1.00 | 15.68 | A | C |
| ATOM | 1388 | CB | ASN | A | 184 | −15.679 | −35.797 | −34.216 | 1.00 | 16.64 | A | C |
| ATOM | 1389 | CG | ASN | A | 184 | −16.978 | −36.426 | −33.740 | 1.00 | 17.82 | A | C |
| ATOM | 1390 | OD1 | ASN | A | 184 | −17.165 | −37.653 | −33.767 | 1.00 | 18.50 | A | O |
| ATOM | 1391 | ND2 | ASN | A | 184 | −17.878 | −35.586 | −33.293 | 1.00 | 18.75 | A | N |
| ATOM | 1392 | C | ASN | A | 184 | −13.291 | −36.194 | −34.751 | 1.00 | 15.70 | A | C |
| ATOM | 1393 | O | ASN | A | 184 | −13.363 | −35.865 | −35.931 | 1.00 | 15.92 | A | O |
| ATOM | 1394 | N | THR | A | 185 | −12.182 | −36.102 | −34.027 | 1.00 | 15.51 | A | N |
| ATOM | 1395 | CA | THR | A | 185 | −10.898 | −35.823 | −34.620 | 1.00 | 14.77 | A | C |
| ATOM | 1396 | CB | THR | A | 185 | −10.251 | −34.603 | −33.976 | 1.00 | 14.28 | A | C |
| ATOM | 1397 | OG1 | THR | A | 185 | −11.137 | −33.476 | −34.101 | 1.00 | 13.52 | A | O |
| ATOM | 1398 | CG2 | THR | A | 185 | −8.912 | −34.341 | −34.633 | 1.00 | 13.98 | A | C |
| ATOM | 1399 | C | THR | A | 185 | −10.039 | −37.035 | −34.382 | 1.00 | 14.98 | A | C |
| ATOM | 1400 | O | THR | A | 185 | −9.666 | −37.310 | −33.243 | 1.00 | 16.15 | A | O |
| ATOM | 1401 | N | PRO | A | 186 | −9.769 | −37.814 | −35.435 | 1.00 | 14.99 | A | N |
| ATOM | 1402 | CA | PRO | A | 186 | −8.988 | −39.026 | −35.190 | 1.00 | 14.73 | A | C |
| ATOM | 1403 | CB | PRO | A | 186 | −9.308 | −39.914 | −36.399 | 1.00 | 14.75 | A | C |
| ATOM | 1404 | CG | PRO | A | 186 | −9.666 | −38.966 | −37.480 | 1.00 | 14.83 | A | C |
| ATOM | 1405 | CD | PRO | A | 186 | −10.245 | −37.732 | −36.828 | 1.00 | 14.83 | A | C |
| ATOM | 1406 | C | PRO | A | 186 | −7.510 | −38.741 | −35.136 | 1.00 | 14.30 | A | C |
| ATOM | 1407 | O | PRO | A | 186 | −7.056 | −37.670 | −35.509 | 1.00 | 14.74 | A | O |
| ATOM | 1408 | N | GLY | A | 187 | −6.778 | −39.711 | −34.630 | 1.00 | 14.35 | A | N |
| ATOM | 1409 | CA | GLY | A | 187 | −5.330 | −39.677 | −34.597 | 1.00 | 14.24 | A | C |
| ATOM | 1410 | C | GLY | A | 187 | −4.707 | −38.787 | −33.543 | 1.00 | 14.00 | A | C |
| ATOM | 1411 | O | GLY | A | 187 | −3.502 | −38.880 | −33.301 | 1.00 | 13.60 | A | O |
| ATOM | 1412 | N | ASP | A | 188 | −5.504 | −37.925 | −32.907 | 1.00 | 13.79 | A | N |
| ATOM | 1413 | CA | ASP | A | 188 | −4.957 | −37.050 | −31.879 | 1.00 | 13.64 | A | C |
| ATOM | 1414 | CB | ASP | A | 188 | −5.532 | −35.633 | −32.008 | 1.00 | 13.33 | A | C |
| ATOM | 1415 | CG | ASP | A | 188 | −6.943 | −35.496 | −31.502 | 1.00 | 13.39 | A | C |
| ATOM | 1416 | OD1 | ASP | A | 188 | −7.707 | −36.462 | −31.383 | 1.00 | 13.27 | A | O |
| ATOM | 1417 | OD2 | ASP | A | 188 | −7.318 | −34.350 | −31.215 | 1.00 | 14.35 | A | O |
| ATOM | 1418 | C | ASP | A | 188 | −5.070 | −37.612 | −30.452 | 1.00 | 13.88 | A | C |
| ATOM | 1419 | O | ASP | A | 188 | −5.619 | −38.712 | −30.208 | 1.00 | 14.39 | A | O |
| ATOM | 1420 | N | ALA | A | 189 | −4.540 | −36.842 | −29.515 | 1.00 | 13.78 | A | N |
| ATOM | 1421 | CA | ALA | A | 189 | −4.466 | −37.257 | −28.144 | 1.00 | 13.80 | A | C |
| ATOM | 1422 | CB | ALA | A | 189 | −3.329 | −38.259 | −27.955 | 1.00 | 13.78 | A | C |
| ATOM | 1423 | C | ALA | A | 189 | −4.288 | −36.053 | −27.242 | 1.00 | 13.69 | A | C |
| ATOM | 1424 | O | ALA | A | 189 | −4.183 | −34.937 | −27.707 | 1.00 | 13.66 | A | O |
| ATOM | 1425 | N | LEU | A | 190 | −4.260 | −36.310 | −25.941 | 1.00 | 14.07 | A | N |
| ATOM | 1426 | CA | LEU | A | 190 | −4.173 | −35.276 | −24.926 | 1.00 | 14.36 | A | C |
| ATOM | 1427 | CB | LEU | A | 190 | −4.778 | −35.797 | −23.621 | 1.00 | 14.90 | A | C |
| ATOM | 1428 | CG | LEU | A | 190 | −4.925 | −34.762 | −22.530 | 1.00 | 15.37 | A | C |
| ATOM | 1429 | CD1 | LEU | A | 190 | −5.581 | −33.503 | −23.056 | 1.00 | 15.75 | A | C |
| ATOM | 1430 | CD2 | LEU | A | 190 | −5.757 | −35.349 | −21.418 | 1.00 | 15.87 | A | C |
| ATOM | 1431 | C | LEU | A | 190 | −2.730 | −34.822 | −24.710 | 1.00 | 13.65 | A | C |
| ATOM | 1432 | O | LEU | A | 190 | −2.470 | −33.616 | −24.670 | 1.00 | 14.14 | A | O |
| ATOM | 1433 | N | ARG | A | 191 | −1.808 | −35.785 | −24.610 | 1.00 | 12.55 | A | N |
| ATOM | 1434 | CA | ARG | A | 191 | −0.373 | −35.501 | −24.585 | 1.00 | 11.67 | A | C |
| ATOM | 1435 | CB | ARG | A | 191 | 0.172 | −35.511 | −23.158 | 1.00 | 11.66 | A | C |
| ATOM | 1436 | CG | ARG | A | 191 | −0.511 | −34.557 | −22.197 | 1.00 | 11.40 | A | C |
| ATOM | 1437 | CD | ARG | A | 191 | 0.195 | −34.508 | −20.853 | 1.00 | 11.14 | A | C |
| ATOM | 1438 | NE | ARG | A | 191 | −0.205 | −35.582 | −19.945 | 1.00 | 10.68 | A | N |
| ATOM | 1439 | CZ | ARG | A | 191 | −1.333 | −35.589 | −19.264 | 1.00 | 10.70 | A | C |
| ATOM | 1440 | NH1 | ARG | A | 191 | −2.205 | −34.590 | −19.381 | 1.00 | 11.00 | A | N |
| ATOM | 1441 | NH2 | ARG | A | 191 | −1.598 | −36.590 | −18.450 | 1.00 | 10.72 | A | N |
| ATOM | 1442 | C | ARG | A | 191 | 0.398 | −36.543 | −25.332 | 1.00 | 11.21 | A | C |
| ATOM | 1443 | O | ARG | A | 191 | −0.009 | −37.712 | −25.424 | 1.00 | 10.54 | A | O |
| ATOM | 1444 | N | SER | A | 192 | 1.555 | −36.128 | −25.807 | 1.00 | 11.04 | A | N |
| ATOM | 1445 | CA | SER | A | 192 | 2.431 | −37.032 | −26.528 | 1.00 | 11.41 | A | C |
| ATOM | 1446 | CB | SER | A | 192 | 2.631 | −36.558 | −27.971 | 1.00 | 11.37 | A | C |
| ATOM | 1447 | OG | SER | A | 192 | 3.803 | −37.111 | −28.558 | 1.00 | 11.38 | A | O |
| ATOM | 1448 | C | SER | A | 192 | 3.765 | −37.098 | −25.819 | 1.00 | 11.56 | A | C |
| ATOM | 1449 | O | SER | A | 192 | 4.463 | −36.094 | −25.698 | 1.00 | 11.58 | A | O |
| ATOM | 1450 | N | MET | A | 193 | 4.127 | −38.289 | −25.384 | 1.00 | 11.67 | A | N |
| ATOM | 1451 | CA | MET | A | 193 | 5.411 | −38.483 | −24.767 | 1.00 | 12.16 | A | C |
| ATOM | 1452 | CB | MET | A | 193 | 5.413 | −39.767 | −23.944 | 1.00 | 12.44 | A | C |
| ATOM | 1453 | CG | MET | A | 193 | 4.355 | −39.828 | −22.870 | 1.00 | 12.51 | A | C |
| ATOM | 1454 | SD | MET | A | 193 | 4.455 | −41.399 | −22.016 | 1.00 | 12.70 | A | S |
| ATOM | 1455 | CE | MET | A | 193 | 6.110 | −41.322 | −21.326 | 1.00 | 13.02 | A | C |
| ATOM | 1456 | C | MET | A | 193 | 6.562 | −38.560 | −25.776 | 1.00 | 12.02 | A | C |
| ATOM | 1457 | O | MET | A | 193 | 7.719 | −38.425 | −25.398 | 1.00 | 11.80 | A | O |
| ATOM | 1458 | N | GLU | A | 194 | 6.292 | −38.847 | −27.033 | 1.00 | 12.27 | A | N |
| ATOM | 1459 | CA | GLU | A | 194 | 7.421 | −38.982 | −27.980 | 1.00 | 12.83 | A | C |
| ATOM | 1460 | CB | GLU | A | 194 | 7.081 | −39.968 | −29.077 | 1.00 | 13.09 | A | C |
| ATOM | 1461 | CG | GLU | A | 194 | 5.903 | −39.511 | −29.951 | 1.00 | 13.52 | A | C |
| ATOM | 1462 | CD | GLU | A | 194 | 5.281 | −40.631 | −30.743 | 1.00 | 13.63 | A | C |
| ATOM | 1463 | OE1 | GLU | A | 194 | 5.799 | −41.773 | −30.651 | 1.00 | 14.89 | A | O |
| ATOM | 1464 | OE2 | GLU | A | 194 | 4.296 | −40.372 | −31.447 | 1.00 | 12.72 | A | O |
| ATOM | 1465 | C | GLU | A | 194 | 7.764 | −37.621 | −28.572 | 1.00 | 13.17 | A | C |
| ATOM | 1466 | O | GLU | A | 194 | 8.872 | −37.403 | −28.987 | 1.00 | 13.22 | A | O |
| ATOM | 1467 | N | ASN | A | 195 | 6.788 | −36.712 | −28.558 | 1.00 | 13.78 | A | N |
| ATOM | 1468 | CA | ASN | A | 195 | 6.905 | −35.373 | −29.097 | 1.00 | 14.09 | A | C |
| ATOM | 1469 | CB | ASN | A | 195 | 6.568 | −35.428 | −30.580 | 1.00 | 13.89 | A | C |
| ATOM | 1470 | CG | ASN | A | 195 | 6.721 | −34.074 | −31.267 | 1.00 | 13.72 | A | C |
| ATOM | 1471 | OD1 | ASN | A | 195 | 7.627 | −33.311 | −30.972 | 1.00 | 13.41 | A | O |
| ATOM | 1472 | ND2 | ASN | A | 195 | 5.834 | −33.786 | −32.198 | 1.00 | 13.75 | A | N |
| ATOM | 1473 | C | ASN | A | 195 | 5.967 | −34.375 | −28.387 | 1.00 | 14.61 | A | C |
| ATOM | 1474 | O | ASN | A | 195 | 4.898 | −34.055 | −28.885 | 1.00 | 15.66 | A | O |
| ATOM | 1475 | N | PRO | A | 196 | 6.347 | −33.900 | −27.209 | 1.00 | 14.75 | A | N |
| ATOM | 1476 | CA | PRO | A | 196 | 5.418 | −33.102 | −26.381 | 1.00 | 14.82 | A | C |
| ATOM | 1477 | CB | PRO | A | 196 | 6.265 | −32.773 | −25.157 | 1.00 | 14.80 | A | C |
| ATOM | 1478 | CG | PRO | A | 196 | 7.180 | −33.963 | −25.022 | 1.00 | 15.30 | A | C |
| ATOM | 1479 | CD | PRO | A | 196 | 7.505 | −34.382 | −26.437 | 1.00 | 15.18 | A | C |
| ATOM | 1480 | C | PRO | A | 196 | 4.894 | −31.812 | −27.012 | 1.00 | 14.89 | A | C |
| ATOM | 1481 | O | PRO | A | 196 | 3.743 | −31.393 | −26.764 | 1.00 | 14.12 | A | O |
| ATOM | 1482 | N | LYS | A | 197 | 5.751 | −31.193 | −27.816 | 1.00 | 15.78 | A | N |
| ATOM | 1483 | CA | LYS | A | 197 | 5.454 | −29.918 | −28.464 | 1.00 | 16.20 | A | C |
| ATOM | 1484 | CB | LYS | A | 197 | 6.607 | −29.476 | −29.345 | 1.00 | 17.65 | A | C |
| ATOM | 1485 | CG | LYS | A | 197 | 7.392 | −28.274 | −28.883 | 1.00 | 19.35 | A | C |
| ATOM | 1486 | CD | LYS | A | 197 | 8.470 | −28.028 | −29.955 | 1.00 | 21.72 | A | C |
| ATOM | 1487 | CE | LYS | A | 197 | 9.427 | −26.872 | −29.652 | 1.00 | 22.98 | A | C |
| ATOM | 1488 | NZ | LYS | A | 197 | 8.975 | −25.622 | −30.336 | 1.00 | 23.87 | A | N |
| ATOM | 1489 | C | LYS | A | 197 | 4.219 | −30.061 | −29.310 | 1.00 | 14.95 | A | C |
| ATOM | 1490 | O | LYS | A | 197 | 3.513 | −29.114 | −29.515 | 1.00 | 13.94 | A | O |
| ATOM | 1491 | N | LEU | A | 198 | 3.956 | −31.257 | −29.791 | 1.00 | 14.64 | A | N |
| ATOM | 1492 | CA | LEU | A | 198 | 2.770 | −31.476 | −30.581 | 1.00 | 15.36 | A | C |
| ATOM | 1493 | CB | LEU | A | 198 | 2.576 | −32.969 | −30.863 | 1.00 | 15.67 | A | C |
| ATOM | 1494 | CG | LEU | A | 198 | 1.296 | −33.521 | −31.533 | 1.00 | 15.57 | A | C |
| ATOM | 1495 | CD1 | LEU | A | 198 | 0.973 | −32.894 | −32.886 | 1.00 | 15.47 | A | C |
| ATOM | 1496 | CD2 | LEU | A | 198 | 1.415 | −35.038 | −31.654 | 1.00 | 15.06 | A | C |
| ATOM | 1497 | C | LEU | A | 198 | 1.554 | −30.888 | −29.906 | 1.00 | 15.80 | A | C |
| ATOM | 1498 | O | LEU | A | 198 | 0.812 | −30.175 | −30.537 | 1.00 | 17.11 | A | O |
| ATOM | 1499 | N | TYR | A | 199 | 1.347 | −31.155 | −28.628 | 1.00 | 16.25 | A | N |
| ATOM | 1500 | CA | TYR | A | 199 | 0.175 | −30.598 | −27.921 | 1.00 | 16.26 | A | C |
| ATOM | 1501 | CB | TYR | A | 199 | −0.650 | −31.738 | −27.300 | 1.00 | 15.53 | A | C |
| ATOM | 1502 | CG | TYR | A | 199 | −1.040 | −32.822 | −28.279 | 1.00 | 14.71 | A | C |
| ATOM | 1503 | CD1 | TYR | A | 199 | −1.966 | −32.579 | −29.279 | 1.00 | 14.13 | A | C |
| ATOM | 1504 | CE1 | TYR | A | 199 | −2.340 | −33.559 | −30.171 | 1.00 | 13.70 | A | C |
| ATOM | 1505 | CZ | TYR | A | 199 | −1.790 | −34.803 | −30.078 | 1.00 | 14.03 | A | C |
| ATOM | 1506 | OH | TYR | A | 199 | −2.164 | −35.782 | −30.956 | 1.00 | 14.59 | A | O |
| ATOM | 1507 | CE2 | TYR | A | 199 | −0.875 | −35.098 | −29.085 | 1.00 | 14.38 | A | C |
| ATOM | 1508 | CD2 | TYR | A | 199 | −0.492 | −34.098 | −28.198 | 1.00 | 14.66 | A | C |
| ATOM | 1509 | C | TYR | A | 199 | 0.572 | −29.551 | −26.857 | 1.00 | 17.22 | A | C |
| ATOM | 1510 | O | TYR | A | 199 | 0.113 | −29.585 | −25.712 | 1.00 | 17.25 | A | O |
| ATOM | 1511 | N | ASN | A | 200 | 1.441 | −28.624 | −27.242 | 1.00 | 18.43 | A | N |
| ATOM | 1512 | CA | ASN | A | 200 | 1.827 | −27.479 | −26.386 | 1.00 | 18.86 | A | C |
| ATOM | 1513 | CB | ASN | A | 200 | 0.631 | −26.532 | −26.215 | 1.00 | 21.02 | A | C |
| ATOM | 1514 | CG | ASN | A | 200 | 0.375 | −25.721 | −27.468 | 1.00 | 23.20 | A | C |
| ATOM | 1515 | OD1 | ASN | A | 200 | 1.312 | −25.396 | −28.219 | 1.00 | 26.27 | A | O |
| ATOM | 1516 | ND2 | ASN | A | 200 | −0.876 | −25.382 | −27.704 | 1.00 | 24.08 | A | N |
| ATOM | 1517 | C | ASN | A | 200 | 2.439 | −27.802 | −25.035 | 1.00 | 17.25 | A | C |
| ATOM | 1518 | O | ASN | A | 200 | 2.142 | −27.171 | −24.036 | 1.00 | 16.12 | A | O |
| ATOM | 1519 | N | GLN | A | 201 | 3.287 | −28.816 | −25.018 | 1.00 | 16.59 | A | N |
| ATOM | 1520 | CA | GLN | A | 201 | 4.176 | −29.045 | −23.898 | 1.00 | 15.78 | A | C |
| ATOM | 1521 | CB | GLN | A | 201 | 4.079 | −30.469 | −23.387 | 1.00 | 15.50 | A | C |
| ATOM | 1522 | CG | GLN | A | 201 | 2.816 | −30.765 | −22.619 | 1.00 | 15.69 | A | C |
| ATOM | 1523 | CD | GLN | A | 201 | 2.783 | −32.192 | −22.089 | 1.00 | 16.07 | A | C |
| ATOM | 1524 | OE1 | GLN | A | 201 | 2.975 | −33.164 | −22.829 | 1.00 | 15.13 | A | O |
| ATOM | 1525 | NE2 | GLN | A | 201 | 2.540 | −32.322 | −20.790 | 1.00 | 16.64 | A | N |
| ATOM | 1526 | C | GLN | A | 201 | 5.602 | −28.747 | −24.336 | 1.00 | 15.44 | A | C |
| ATOM | 1527 | O | GLN | A | 201 | 6.000 | −29.038 | −25.453 | 1.00 | 14.82 | A | O |
| ATOM | 1528 | N | PRO | A | 202 | 6.365 | −28.113 | −23.463 | 1.00 | 16.00 | A | N |
| ATOM | 1529 | CA | PRO | A | 202 | 7.760 | −27.900 | −23.754 | 1.00 | 16.63 | A | C |
| ATOM | 1530 | CB | PRO | A | 202 | 8.227 | −27.048 | −22.586 | 1.00 | 16.94 | A | C |
| ATOM | 1531 | CG | PRO | A | 202 | 7.226 | −27.316 | −21.507 | 1.00 | 17.12 | A | C |
| ATOM | 1532 | CD | PRO | A | 202 | 5.949 | −27.401 | −22.249 | 1.00 | 16.49 | A | C |
| ATOM | 1533 | C | PRO | A | 202 | 8.482 | −29.203 | −23.734 | 1.00 | 16.91 | A | C |
| ATOM | 1534 | O | PRO | A | 202 | 8.043 | −30.115 | −23.030 | 1.00 | 17.59 | A | O |
| ATOM | 1535 | N | ASP | A | 203 | 9.563 | −29.286 | −24.497 | 1.00 | 16.60 | A | N |
| ATOM | 1536 | CA | ASP | A | 203 | 10.369 | −30.469 | −24.532 | 1.00 | 17.19 | A | C |
| ATOM | 1537 | CB | ASP | A | 203 | 10.337 | −31.157 | −25.911 | 1.00 | 17.40 | A | C |
| ATOM | 1538 | CG | ASP | A | 203 | 10.885 | −30.299 | −27.056 | 1.00 | 17.15 | A | C |
| ATOM | 1539 | OD1 | ASP | A | 203 | 11.307 | −29.124 | −26.880 | 1.00 | 16.43 | A | O |
| ATOM | 1540 | OD2 | ASP | A | 203 | 10.849 | −30.843 | −28.180 | 1.00 | 16.95 | A | O |
| ATOM | 1541 | C | ASP | A | 203 | 11.791 | −30.209 | −24.112 | 1.00 | 18.70 | A | C |
| ATOM | 1542 | O | ASP | A | 203 | 12.676 | −31.022 | −24.429 | 1.00 | 19.51 | A | O |
| ATOM | 1543 | N | ARG | A | 204 | 12.020 | −29.116 | −23.381 | 1.00 | 19.82 | A | N |
| ATOM | 1544 | CA | ARG | A | 204 | 13.245 | −28.985 | −22.593 | 1.00 | 20.95 | A | C |
| ATOM | 1545 | CB | ARG | A | 204 | 14.421 | −28.585 | −23.458 | 1.00 | 23.55 | A | C |
| ATOM | 1546 | CG | ARG | A | 204 | 14.337 | −27.186 | −24.020 | 1.00 | 27.12 | A | C |
| ATOM | 1547 | CD | ARG | A | 204 | 15.383 | −27.032 | −25.090 | 1.00 | 30.35 | A | C |
| ATOM | 1548 | NE | ARG | A | 204 | 14.974 | −26.129 | −26.152 | 1.00 | 34.09 | A | N |
| ATOM | 1549 | CZ | ARG | A | 204 | 15.738 | −25.158 | −26.639 | 1.00 | 37.57 | A | C |
| ATOM | 1550 | NH1 | ARG | A | 204 | 16.955 | −24.947 | −26.119 | 1.00 | 38.53 | A | N |
| ATOM | 1551 | NH2 | ARG | A | 204 | 15.277 | −24.384 | −27.633 | 1.00 | 37.08 | A | N |
| ATOM | 1552 | C | ARG | A | 204 | 13.103 | −27.993 | −21.473 | 1.00 | 19.87 | A | C |
| ATOM | 1553 | O | ARG | A | 204 | 12.316 | −27.060 | −21.576 | 1.00 | 18.88 | A | O |
| ATOM | 1554 | N | TYR | A | 205 | 13.886 | −28.202 | −20.417 | 1.00 | 19.61 | A | N |
| ATOM | 1555 | CA | TYR | A | 205 | 13.838 | −27.375 | −19.190 | 1.00 | 20.53 | A | C |
| ATOM | 1556 | CB | TYR | A | 205 | 15.060 | −27.682 | −18.280 | 1.00 | 20.73 | A | C |
| ATOM | 1557 | CG | TYR | A | 205 | 14.926 | −27.129 | −16.882 | 1.00 | 20.80 | A | C |
| ATOM | 1558 | CD1 | TYR | A | 205 | 13.810 | −27.430 | −16.109 | 1.00 | 21.37 | A | C |
| ATOM | 1559 | CE1 | TYR | A | 205 | 13.660 | −26.932 | −14.832 | 1.00 | 21.21 | A | C |
| ATOM | 1560 | CZ | TYR | A | 205 | 14.641 | −26.148 | −14.306 | 1.00 | 20.82 | A | C |
| ATOM | 1561 | OH | TYR | A | 205 | 14.447 | −25.681 | −13.053 | 1.00 | 20.22 | A | O |
| ATOM | 1562 | CE2 | TYR | A | 205 | 15.776 | −25.857 | −15.025 | 1.00 | 20.57 | A | C |
| ATOM | 1563 | CD2 | TYR | A | 205 | 15.908 | −26.336 | −16.317 | 1.00 | 20.84 | A | C |
| ATOM | 1564 | C | TYR | A | 205 | 13.749 | −25.859 | −19.440 | 1.00 | 20.17 | A | C |
| ATOM | 1565 | O | TYR | A | 205 | 12.904 | −25.167 | −18.875 | 1.00 | 18.72 | A | O |
| ATOM | 1566 | N | GLN | A | 206 | 14.603 | −25.377 | −20.335 | 1.00 | 21.34 | A | N |
| ATOM | 1567 | CA | GLN | A | 206 | 14.715 | −23.948 | −20.671 | 1.00 | 22.58 | A | C |
| ATOM | 1568 | CB | GLN | A | 206 | 15.767 | −23.764 | −21.757 | 1.00 | 24.80 | A | C |
| ATOM | 1569 | CG | GLN | A | 206 | 17.129 | −24.352 | −21.385 | 1.00 | 28.21 | A | C |
| ATOM | 1570 | CD | GLN | A | 206 | 17.413 | −25.682 | −22.073 | 1.00 | 31.20 | A | C |
| ATOM | 1571 | OE1 | GLN | A | 206 | 16.776 | −26.702 | −21.784 | 1.00 | 33.57 | A | O |
| ATOM | 1572 | NE2 | GLN | A | 206 | 18.366 | −25.673 | −23.013 | 1.00 | 34.48 | A | N |
| ATOM | 1573 | C | GLN | A | 206 | 13.413 | −23.257 | −21.115 | 1.00 | 21.39 | A | C |
| ATOM | 1574 | O | GLN | A | 206 | 13.335 | −22.012 | −21.080 | 1.00 | 21.09 | A | O |
| ATOM | 1575 | N | ASP | A | 207 | 12.419 | −24.061 | −21.530 | 1.00 | 19.47 | A | N |
| ATOM | 1576 | CA | ASP | A | 207 | 11.158 | −23.578 | −22.108 | 1.00 | 17.35 | A | C |
| ATOM | 1577 | CB | ASP | A | 207 | 10.898 | −24.207 | −23.492 | 1.00 | 17.00 | A | C |
| ATOM | 1578 | CG | ASP | A | 207 | 11.998 | −23.917 | −24.494 | 1.00 | 16.98 | A | C |
| ATOM | 1579 | OD1 | ASP | A | 207 | 12.681 | −22.892 | −24.342 | 1.00 | 17.05 | A | O |
| ATOM | 1580 | OD2 | ASP | A | 207 | 12.192 | −24.713 | −25.433 | 1.00 | 16.83 | A | O |
| ATOM | 1581 | C | ASP | A | 207 | 10.007 | −23.911 | −21.198 | 1.00 | 15.93 | A | C |
| ATOM | 1582 | O | ASP | A | 207 | 8.858 | −23.867 | −21.605 | 1.00 | 15.49 | A | O |
| ATOM | 1583 | N | ARG | A | 208 | 10.299 | −24.246 | −19.960 | 1.00 | 14.96 | A | N |
| ATOM | 1584 | CA | ARG | A | 208 | 9.222 | −24.567 | −19.046 | 1.00 | 14.60 | A | C |
| ATOM | 1585 | CB | ARG | A | 208 | 9.746 | −25.100 | −17.708 | 1.00 | 14.67 | A | C |
| ATOM | 1586 | CG | ARG | A | 208 | 10.371 | −24.035 | −16.833 | 1.00 | 14.67 | A | C |
| ATOM | 1587 | CD | ARG | A | 208 | 11.284 | −24.671 | −15.819 | 1.00 | 14.86 | A | C |
| ATOM | 1588 | NE | ARG | A | 208 | 12.055 | −23.638 | −15.162 | 1.00 | 15.29 | A | N |
| ATOM | 1589 | CZ | ARG | A | 208 | 13.206 | −23.171 | −15.614 | 1.00 | 16.10 | A | C |
| ATOM | 1590 | NH1 | ARG | A | 208 | 13.731 | −23.649 | −16.721 | 1.00 | 17.73 | A | N |
| ATOM | 1591 | NH2 | ARG | A | 208 | 13.845 | −22.226 | −14.964 | 1.00 | 16.00 | A | N |
| ATOM | 1592 | C | ARG | A | 208 | 8.373 | −23.358 | −18.777 | 1.00 | 13.74 | A | C |
| ATOM | 1593 | O | ARG | A | 208 | 8.860 | −22.278 | −18.641 | 1.00 | 12.69 | A | O |
| ATOM | 1594 | N | TYR | A | 209 | 7.081 | −23.595 | −18.688 | 1.00 | 14.32 | A | N |
| ATOM | 1595 | CA | TYR | A | 209 | 6.136 | −22.639 | −18.140 | 1.00 | 14.70 | A | C |
| ATOM | 1596 | CB | TYR | A | 209 | 4.711 | −23.201 | −18.179 | 1.00 | 14.05 | A | C |
| ATOM | 1597 | CG | TYR | A | 209 | 3.623 | −22.377 | −17.503 | 1.00 | 13.32 | A | C |
| ATOM | 1598 | CD1 | TYR | A | 209 | 3.136 | −21.228 | −18.076 | 1.00 | 13.15 | A | C |
| ATOM | 1599 | CE1 | TYR | A | 209 | 2.097 | −20.516 | −17.498 | 1.00 | 12.94 | A | C |
| ATOM | 1600 | CZ | TYR | A | 209 | 1.528 | −20.944 | −16.330 | 1.00 | 13.00 | A | C |
| ATOM | 1601 | OH | TYR | A | 209 | 0.493 | −20.212 | −15.726 | 1.00 | 12.90 | A | O |
| ATOM | 1602 | CE2 | TYR | A | 209 | 2.012 | −22.088 | −15.741 | 1.00 | 12.96 | A | C |
| ATOM | 1603 | CD2 | TYR | A | 209 | 3.043 | −22.796 | −16.334 | 1.00 | 12.99 | A | C |
| ATOM | 1604 | C | TYR | A | 209 | 6.513 | −22.419 | −16.714 | 1.00 | 15.33 | A | C |
| ATOM | 1605 | O | TYR | A | 209 | 6.889 | −23.363 | −16.023 | 1.00 | 15.11 | A | O |
| ATOM | 1606 | N | THR | A | 210 | 6.359 | −21.175 | −16.286 | 1.00 | 16.15 | A | N |
| ATOM | 1607 | CA | THR | A | 210 | 6.796 | −20.737 | −14.986 | 1.00 | 17.20 | A | C |
| ATOM | 1608 | CB | THR | A | 210 | 8.162 | −19.996 | −15.135 | 1.00 | 18.44 | A | C |
| ATOM | 1609 | OG1 | THR | A | 210 | 8.620 | −19.652 | −13.838 | 1.00 | 22.63 | A | O |
| ATOM | 1610 | CG2 | THR | A | 210 | 8.092 | −18.692 | −15.962 | 1.00 | 18.69 | A | C |
| ATOM | 1611 | C | THR | A | 210 | 5.720 | −19.937 | −14.186 | 1.00 | 16.04 | A | C |
| ATOM | 1612 | O | THR | A | 210 | 5.983 | −19.437 | −13.110 | 1.00 | 15.79 | A | O |
| ATOM | 1613 | N | GLY | A | 211 | 4.497 | −19.885 | −14.697 | 1.00 | 16.00 | A | N |
| ATOM | 1614 | CA | GLY | A | 211 | 3.389 | −19.131 | −14.079 | 1.00 | 16.15 | A | C |
| ATOM | 1615 | C | GLY | A | 211 | 2.576 | −19.933 | −13.066 | 1.00 | 16.36 | A | C |
| ATOM | 1616 | O | GLY | A | 211 | 2.959 | −21.048 | −12.712 | 1.00 | 17.38 | A | O |
| ATOM | 1617 | N | PRO | A | 212 | 1.458 | −19.375 | −12.575 | 1.00 | 15.90 | A | N |
| ATOM | 1618 | CA | PRO | A | 212 | 0.825 | −19.956 | −11.405 | 1.00 | 15.80 | A | C |
| ATOM | 1619 | CB | PRO | A | 212 | 0.200 | −18.756 | −10.716 | 1.00 | 15.94 | A | C |
| ATOM | 1620 | CG | PRO | A | 212 | −0.123 | −17.809 | −11.817 | 1.00 | 16.11 | A | C |
| ATOM | 1621 | CD | PRO | A | 212 | 0.825 | −18.103 | −12.955 | 1.00 | 16.30 | A | C |
| ATOM | 1622 | C | PRO | A | 212 | −0.258 | −20.921 | −11.730 | 1.00 | 16.41 | A | C |
| ATOM | 1623 | O | PRO | A | 212 | −0.656 | −21.692 | −10.857 | 1.00 | 16.72 | A | O |
| ATOM | 1624 | N | SER | A | 213 | −0.724 | −20.898 | −12.973 | 1.00 | 16.53 | A | N |
| ATOM | 1625 | CA | SER | A | 213 | −1.837 | −21.716 | −13.358 | 1.00 | 16.75 | A | C |
| ATOM | 1626 | CB | SER | A | 213 | −2.284 | −21.316 | −14.748 | 1.00 | 17.51 | A | C |
| ATOM | 1627 | OG | SER | A | 213 | −3.661 | −21.579 | −14.853 | 1.00 | 18.94 | A | O |
| ATOM | 1628 | C | SER | A | 213 | −1.481 | −23.209 | −13.345 | 1.00 | 17.13 | A | C |
| ATOM | 1629 | O | SER | A | 213 | −0.285 | −23.588 | −13.469 | 1.00 | 17.29 | A | O |
| ATOM | 1630 | N | ASP | A | 214 | −2.513 | −24.059 | −13.209 | 1.00 | 16.27 | A | N |
| ATOM | 1631 | CA | ASP | A | 214 | −2.329 | −25.508 | −13.237 | 1.00 | 15.08 | A | C |
| ATOM | 1632 | CB | ASP | A | 214 | −1.851 | −25.926 | −14.630 | 1.00 | 15.42 | A | C |
| ATOM | 1633 | CG | ASP | A | 214 | −1.721 | −27.430 | −14.796 | 1.00 | 15.51 | A | C |
| ATOM | 1634 | OD1 | ASP | A | 214 | −2.543 | −28.180 | −14.216 | 1.00 | 16.01 | A | O |
| ATOM | 1635 | OD2 | ASP | A | 214 | −0.798 | −27.862 | −15.528 | 1.00 | 14.86 | A | O |
| ATOM | 1636 | C | ASP | A | 214 | −1.305 | −25.901 | −12.213 | 1.00 | 14.27 | A | C |
| ATOM | 1637 | O | ASP | A | 214 | −0.375 | −26.606 | −12.522 | 1.00 | 13.66 | A | O |
| ATOM | 1638 | N | ASN | A | 215 | −1.466 | −25.409 | −10.995 | 1.00 | 14.22 | A | N |
| ATOM | 1639 | CA | ASN | A | 215 | −0.507 | −25.653 | −9.924 | 1.00 | 14.30 | A | C |
| ATOM | 1640 | CB | ASN | A | 215 | −0.702 | −27.060 | −9.334 | 1.00 | 13.77 | A | C |
| ATOM | 1641 | CG | ASN | A | 215 | −1.959 | −27.172 | −8.472 | 1.00 | 13.33 | A | C |
| ATOM | 1642 | OD1 | ASN | A | 215 | −2.241 | −26.309 | −7.648 | 1.00 | 13.58 | A | O |
| ATOM | 1643 | ND2 | ASN | A | 215 | −2.705 | −28.225 | −8.658 | 1.00 | 12.58 | A | N |
| ATOM | 1644 | C | ASN | A | 215 | 0.944 | −25.479 | −10.367 | 1.00 | 15.43 | A | C |
| ATOM | 1645 | O | ASN | A | 215 | 1.768 | −26.360 | −10.129 | 1.00 | 17.15 | A | O |
| ATOM | 1646 | N | GLY | A | 216 | 1.267 | −24.372 | −11.033 | 1.00 | 15.63 | A | N |
| ATOM | 1647 | CA | GLY | A | 216 | 2.641 | −24.132 | −11.479 | 1.00 | 15.35 | A | C |
| ATOM | 1648 | C | GLY | A | 216 | 3.070 | −24.980 | −12.666 | 1.00 | 15.58 | A | C |
| ATOM | 1649 | O | GLY | A | 216 | 4.219 | −25.467 | −12.737 | 1.00 | 16.25 | A | O |
| ATOM | 1650 | N | GLY | A | 217 | 2.158 | −25.181 | −13.611 | 1.00 | 15.34 | A | N |
| ATOM | 1651 | CA | GLY | A | 217 | 2.524 | −25.840 | −14.884 | 1.00 | 14.91 | A | C |
| ATOM | 1652 | C | GLY | A | 217 | 2.633 | −27.352 | −14.829 | 1.00 | 14.14 | A | C |
| ATOM | 1653 | O | GLY | A | 217 | 3.387 | −27.969 | −15.596 | 1.00 | 14.67 | A | O |
| ATOM | 1654 | N | VAL | A | 218 | 1.829 | −27.949 | −13.972 | 1.00 | 12.95 | A | N |
| ATOM | 1655 | CA | VAL | A | 218 | 2.024 | −29.338 | −13.605 | 1.00 | 12.93 | A | C |
| ATOM | 1656 | CB | VAL | A | 218 | 1.198 | −29.641 | −12.327 | 1.00 | 12.94 | A | C |
| ATOM | 1657 | CG1 | VAL | A | 218 | 0.249 | −30.815 | −12.469 | 1.00 | 12.92 | A | C |
| ATOM | 1658 | CG2 | VAL | A | 218 | 2.142 | −29.767 | −11.133 | 1.00 | 12.90 | A | C |
| ATOM | 1659 | C | VAL | A | 218 | 1.825 | −30.284 | −14.785 | 1.00 | 12.86 | A | C |
| ATOM | 1660 | O | VAL | A | 218 | 2.681 | −31.110 | −15.074 | 1.00 | 13.09 | A | O |
| ATOM | 1661 | N | HIS | A | 219 | 0.733 | −30.092 | −15.512 | 1.00 | 12.63 | A | N |
| ATOM | 1662 | CA | HIS | A | 219 | 0.478 | −30.819 | −16.744 | 1.00 | 12.21 | A | C |
| ATOM | 1663 | CB | HIS | A | 219 | −1.019 | −30.845 | −17.028 | 1.00 | 12.29 | A | C |
| ATOM | 1664 | CG | HIS | A | 219 | −1.812 | −31.473 | −15.932 | 1.00 | 12.04 | A | C |
| ATOM | 1665 | ND1 | HIS | A | 219 | −2.339 | −30.747 | −14.895 | 1.00 | 11.93 | A | N |
| ATOM | 1666 | CE1 | HIS | A | 219 | −2.961 | −31.562 | −14.064 | 1.00 | 12.18 | A | C |
| ATOM | 1667 | NE2 | HIS | A | 219 | −2.828 | −32.794 | −14.514 | 1.00 | 12.05 | A | N |
| ATOM | 1668 | CD2 | HIS | A | 219 | −2.113 | −32.765 | −15.682 | 1.00 | 11.89 | A | C |
| ATOM | 1669 | C | HIS | A | 219 | 1.167 | −30.213 | −17.943 | 1.00 | 11.95 | A | C |
| ATOM | 1670 | O | HIS | A | 219 | 1.170 | −30.826 | −19.008 | 1.00 | 11.86 | A | O |
| ATOM | 1671 | N | ILE | A | 220 | 1.725 | −29.015 | −17.805 | 1.00 | 11.44 | A | N |
| ATOM | 1672 | CA | ILE | A | 220 | 2.414 | −28.443 | −18.914 | 1.00 | 11.45 | A | C |
| ATOM | 1673 | CB | ILE | A | 220 | 2.384 | −26.927 | −18.838 | 1.00 | 11.68 | A | C |
| ATOM | 1674 | CG1 | ILE | A | 220 | 0.951 | −26.446 | −19.053 | 1.00 | 12.04 | A | C |
| ATOM | 1675 | CD1 | ILE | A | 220 | 0.737 | −24.988 | −18.653 | 1.00 | 12.23 | A | C |
| ATOM | 1676 | CG2 | ILE | A | 220 | 3.293 | −26.302 | −19.894 | 1.00 | 11.57 | A | C |
| ATOM | 1677 | C | ILE | A | 220 | 3.841 | −28.999 | −18.991 | 1.00 | 11.72 | A | C |
| ATOM | 1678 | O | ILE | A | 220 | 4.228 | −29.616 | −20.020 | 1.00 | 11.22 | A | O |
| ATOM | 1679 | N | ASN | A | 221 | 4.601 | −28.809 | −17.897 | 1.00 | 11.49 | A | N |
| ATOM | 1680 | CA | ASN | A | 221 | 6.051 | −29.113 | −17.882 | 1.00 | 11.39 | A | C |
| ATOM | 1681 | CB | ASN | A | 221 | 6.761 | −28.288 | −16.820 | 1.00 | 11.36 | A | C |
| ATOM | 1682 | CG | ASN | A | 221 | 6.457 | −26.809 | −16.934 | 1.00 | 11.22 | A | C |
| ATOM | 1683 | OD1 | ASN | A | 221 | 6.580 | −26.185 | −17.993 | 1.00 | 11.34 | A | O |
| ATOM | 1684 | ND2 | ASN | A | 221 | 6.032 | −26.250 | −15.851 | 1.00 | 11.20 | A | N |
| ATOM | 1685 | C | ASN | A | 221 | 6.413 | −30.581 | −17.667 | 1.00 | 11.36 | A | C |
| ATOM | 1686 | O | ASN | A | 221 | 7.587 | −30.933 | −17.639 | 1.00 | 11.44 | A | O |
| ATOM | 1687 | N | SER | A | 222 | 5.403 | −31.442 | −17.547 | 1.00 | 11.25 | A | N |
| ATOM | 1688 | CA | SER | A | 222 | 5.638 | −32.877 | −17.417 | 1.00 | 10.81 | A | C |
| ATOM | 1689 | CB | SER | A | 222 | 4.345 | −33.617 | −17.026 | 1.00 | 10.68 | A | C |
| ATOM | 1690 | OG | SER | A | 222 | 3.340 | −33.460 | −17.996 | 1.00 | 10.76 | A | O |
| ATOM | 1691 | C | SER | A | 222 | 6.217 | −33.444 | −18.689 | 1.00 | 10.49 | A | C |
| ATOM | 1692 | O | SER | A | 222 | 6.888 | −34.485 | −18.652 | 1.00 | 10.48 | A | O |
| ATOM | 1693 | N | GLY | A | 223 | 5.946 | −32.771 | −19.810 | 1.00 | 10.19 | A | N |
| ATOM | 1694 | CA | GLY | A | 223 | 6.413 | −33.226 | −21.107 | 1.00 | 10.08 | A | C |
| ATOM | 1695 | C | GLY | A | 223 | 7.927 | −33.301 | −21.164 | 1.00 | 10.06 | A | C |
| ATOM | 1696 | O | GLY | A | 223 | 8.491 | −34.143 | −21.859 | 1.00 | 9.81 | A | O |
| ATOM | 1697 | N | ILE | A | 224 | 8.578 | −32.431 | −20.398 | 1.00 | 10.10 | A | N |
| ATOM | 1698 | CA | ILE | A | 224 | 10.020 | −32.426 | −20.317 | 1.00 | 10.23 | A | C |
| ATOM | 1699 | CB | ILE | A | 224 | 10.519 | −31.272 | −19.422 | 1.00 | 10.49 | A | C |
| ATOM | 1700 | CG1 | ILE | A | 224 | 10.159 | −29.913 | −20.031 | 1.00 | 10.32 | A | C |
| ATOM | 1701 | CD1 | ILE | A | 224 | 9.939 | −28.841 | −18.985 | 1.00 | 10.40 | A | C |
| ATOM | 1702 | CG2 | ILE | A | 224 | 12.037 | −31.376 | −19.189 | 1.00 | 10.68 | A | C |
| ATOM | 1703 | C | ILE | A | 224 | 10.505 | −33.749 | −19.756 | 1.00 | 10.23 | A | C |
| ATOM | 1704 | O | ILE | A | 224 | 11.364 | −34.386 | −20.336 | 1.00 | 9.86 | A | O |
| ATOM | 1705 | N | ASN | A | 225 | 9.949 | −34.164 | −18.625 | 1.00 | 10.63 | A | N |
| ATOM | 1706 | CA | ASN | A | 225 | 10.323 | −35.454 | −18.082 | 1.00 | 11.23 | A | C |
| ATOM | 1707 | CB | ASN | A | 225 | 9.905 | −35.631 | −16.640 | 1.00 | 11.81 | A | C |
| ATOM | 1708 | CG | ASN | A | 225 | 10.579 | −36.830 | −15.998 | 1.00 | 12.52 | A | C |
| ATOM | 1709 | OD1 | ASN | A | 225 | 9.945 | −37.827 | −15.680 | 1.00 | 13.05 | A | O |
| ATOM | 1710 | ND2 | ASN | A | 225 | 11.883 | −36.753 | −15.847 | 1.00 | 12.97 | A | N |
| ATOM | 1711 | C | ASN | A | 225 | 9.780 | −36.617 | −18.879 | 1.00 | 11.45 | A | C |
| ATOM | 1712 | O | ASN | A | 225 | 10.456 | −37.629 | −18.972 | 1.00 | 11.51 | A | O |
| ATOM | 1713 | N | ASN | A | 226 | 8.575 | −36.500 | −19.457 | 1.00 | 11.45 | A | N |
| ATOM | 1714 | CA | ASN | A | 226 | 8.081 | −37.559 | −20.356 | 1.00 | 11.23 | A | C |
| ATOM | 1715 | CB | ASN | A | 226 | 6.650 | −37.267 | −20.854 | 1.00 | 11.25 | A | C |
| ATOM | 1716 | CG | ASN | A | 226 | 5.584 | −37.417 | −19.761 | 1.00 | 11.15 | A | C |
| ATOM | 1717 | OD1 | ASN | A | 226 | 5.901 | −37.722 | −18.614 | 1.00 | 10.97 | A | O |
| ATOM | 1718 | ND2 | ASN | A | 226 | 4.313 | −37.165 | −20.112 | 1.00 | 11.00 | A | N |
| ATOM | 1719 | C | ASN | A | 226 | 9.039 | −37.761 | −21.539 | 1.00 | 11.16 | A | C |
| ATOM | 1720 | O | ASN | A | 226 | 9.376 | −38.871 | −21.901 | 1.00 | 10.70 | A | O |
| ATOM | 1721 | N | LYS | A | 227 | 9.501 | −36.669 | −22.125 | 1.00 | 11.75 | A | N |
| ATOM | 1722 | CA | LYS | A | 227 | 10.371 | −36.759 | −23.271 | 1.00 | 12.42 | A | C |
| ATOM | 1723 | CB | LYS | A | 227 | 10.731 | −35.370 | −23.764 | 1.00 | 12.84 | A | C |
| ATOM | 1724 | CG | LYS | A | 227 | 11.611 | −35.359 | −24.990 | 1.00 | 13.61 | A | C |
| ATOM | 1725 | CD | LYS | A | 227 | 10.945 | −35.898 | −26.266 | 1.00 | 14.22 | A | C |
| ATOM | 1726 | CE | LYS | A | 227 | 12.014 | −36.340 | −27.269 | 1.00 | 15.09 | A | C |
| ATOM | 1727 | NZ | LYS | A | 227 | 11.580 | −36.298 | −28.698 | 1.00 | 15.81 | A | N |
| ATOM | 1728 | C | LYS | A | 227 | 11.612 | −37.562 | −22.904 | 1.00 | 12.89 | A | C |
| ATOM | 1729 | O | LYS | A | 227 | 12.015 | −38.460 | −23.641 | 1.00 | 13.47 | A | O |
| ATOM | 1730 | N | ALA | A | 228 | 12.193 | −37.269 | −21.740 | 1.00 | 12.98 | A | N |
| ATOM | 1731 | CA | ALA | A | 228 | 13.335 | −38.036 | −21.243 | 1.00 | 12.64 | A | C |
| ATOM | 1732 | CB | ALA | A | 228 | 13.813 | −37.442 | −19.926 | 1.00 | 12.73 | A | C |
| ATOM | 1733 | C | ALA | A | 228 | 13.068 | −39.558 | −21.096 | 1.00 | 12.18 | A | C |
| ATOM | 1734 | O | ALA | A | 228 | 13.905 | −40.380 | −21.471 | 1.00 | 12.38 | A | O |
| ATOM | 1735 | N | PHE | A | 229 | 11.914 | −39.929 | −20.545 | 1.00 | 11.74 | A | N |
| ATOM | 1736 | CA | PHE | A | 229 | 11.555 | −41.334 | −20.434 | 1.00 | 11.22 | A | C |
| ATOM | 1737 | CB | PHE | A | 229 | 10.240 | −41.554 | −19.676 | 1.00 | 10.83 | A | C |
| ATOM | 1738 | CG | PHE | A | 229 | 9.932 | −42.999 | −19.513 | 1.00 | 10.75 | A | C |
| ATOM | 1739 | CD1 | PHE | A | 229 | 10.570 | −43.735 | −18.552 | 1.00 | 10.84 | A | C |
| ATOM | 1740 | CE1 | PHE | A | 229 | 10.358 | −45.093 | −18.457 | 1.00 | 11.22 | A | C |
| ATOM | 1741 | CZ | PHE | A | 229 | 9.505 | −45.741 | −19.351 | 1.00 | 10.84 | A | C |
| ATOM | 1742 | CE2 | PHE | A | 229 | 8.890 | −45.023 | −20.333 | 1.00 | 10.64 | A | C |
| ATOM | 1743 | CD2 | PHE | A | 229 | 9.104 | −43.659 | −20.409 | 1.00 | 10.89 | A | C |
| ATOM | 1744 | C | PHE | A | 229 | 11.470 | −41.957 | −21.827 | 1.00 | 11.34 | A | C |
| ATOM | 1745 | O | PHE | A | 229 | 11.985 | −43.054 | −22.072 | 1.00 | 11.16 | A | O |
| ATOM | 1746 | N | TYR | A | 230 | 10.831 | −41.248 | −22.750 | 1.00 | 11.53 | A | N |
| ATOM | 1747 | CA | TYR | A | 230 | 10.803 | −41.695 | −24.136 | 1.00 | 11.74 | A | C |
| ATOM | 1748 | CB | TYR | A | 230 | 10.119 | −40.669 | −25.062 | 1.00 | 11.82 | A | C |
| ATOM | 1749 | CG | TYR | A | 230 | 10.185 | −41.060 | −26.522 | 1.00 | 11.70 | A | C |
| ATOM | 1750 | CD1 | TYR | A | 230 | 9.407 | −42.076 | −27.041 | 1.00 | 11.41 | A | C |
| ATOM | 1751 | CE1 | TYR | A | 230 | 9.497 | −42.427 | −28.377 | 1.00 | 11.72 | A | C |
| ATOM | 1752 | CZ | TYR | A | 230 | 10.387 | −41.744 | −29.222 | 1.00 | 12.00 | A | C |
| ATOM | 1753 | OH | TYR | A | 230 | 10.553 | −41.983 | −30.564 | 1.00 | 11.37 | A | O |
| ATOM | 1754 | CE2 | TYR | A | 230 | 11.161 | −40.741 | −28.709 | 1.00 | 12.22 | A | C |
| ATOM | 1755 | CD2 | TYR | A | 230 | 11.058 | −40.409 | −27.367 | 1.00 | 12.28 | A | C |
| ATOM | 1756 | C | TYR | A | 230 | 12.200 | −42.005 | −24.645 | 1.00 | 11.45 | A | C |
| ATOM | 1757 | O | TYR | A | 230 | 12.438 | −43.097 | −25.119 | 1.00 | 11.78 | A | O |
| ATOM | 1758 | N | LEU | A | 231 | 13.114 | −41.058 | −24.519 | 1.00 | 11.40 | A | N |
| ATOM | 1759 | CA | LEU | A | 231 | 14.473 | −41.254 | −24.997 | 1.00 | 11.79 | A | C |
| ATOM | 1760 | CB | LEU | A | 231 | 15.288 | −39.996 | −24.808 | 1.00 | 11.69 | A | C |
| ATOM | 1761 | CG | LEU | A | 231 | 14.814 | −38.855 | −25.698 | 1.00 | 11.76 | A | C |
| ATOM | 1762 | CD1 | LEU | A | 231 | 15.341 | −37.549 | −25.148 | 1.00 | 11.92 | A | C |
| ATOM | 1763 | CD2 | LEU | A | 231 | 15.246 | −39.049 | −27.144 | 1.00 | 11.76 | A | C |
| ATOM | 1764 | C | LEU | A | 231 | 15.190 | −42.435 | −24.338 | 1.00 | 12.58 | A | C |
| ATOM | 1765 | O | LEU | A | 231 | 15.881 | −43.196 | −25.035 | 1.00 | 12.49 | A | O |
| ATOM | 1766 | N | ILE | A | 232 | 15.003 | −42.611 | −23.021 | 1.00 | 13.10 | A | N |
| ATOM | 1767 | CA | ILE | A | 232 | 15.600 | −43.750 | −22.306 | 1.00 | 13.31 | A | C |
| ATOM | 1768 | CB | ILE | A | 232 | 15.493 | −43.616 | −20.781 | 1.00 | 13.13 | A | C |
| ATOM | 1769 | CG1 | ILE | A | 232 | 16.478 | −42.569 | −20.316 | 1.00 | 13.85 | A | C |
| ATOM | 1770 | CD1 | ILE | A | 232 | 16.013 | −41.724 | −19.150 | 1.00 | 14.38 | A | C |
| ATOM | 1771 | CG2 | ILE | A | 232 | 15.890 | −44.889 | −20.075 | 1.00 | 12.88 | A | C |
| ATOM | 1772 | C | ILE | A | 232 | 15.048 | −45.077 | −22.764 | 1.00 | 14.10 | A | C |
| ATOM | 1773 | O | ILE | A | 232 | 15.825 | −46.032 | −22.986 | 1.00 | 14.59 | A | O |
| ATOM | 1774 | N | ALA | A | 233 | 13.727 | −45.158 | −22.924 | 1.00 | 15.20 | A | N |
| ATOM | 1775 | CA | ALA | A | 233 | 13.080 | −46.436 | −23.335 | 1.00 | 15.74 | A | C |
| ATOM | 1776 | CB | ALA | A | 233 | 11.598 | −46.388 | −23.053 | 1.00 | 15.72 | A | C |
| ATOM | 1777 | C | ALA | A | 233 | 13.306 | −46.738 | −24.814 | 1.00 | 16.24 | A | C |
| ATOM | 1778 | O | ALA | A | 233 | 13.765 | −47.824 | −25.156 | 1.00 | 16.87 | A | O |
| ATOM | 1779 | N | GLN | A | 234 | 13.030 | −45.743 | −25.661 | 1.00 | 15.67 | A | N |
| ATOM | 1780 | CA | GLN | A | 234 | 12.883 | −45.933 | −27.091 | 1.00 | 15.40 | A | C |
| ATOM | 1781 | CB | GLN | A | 234 | 11.714 | −45.074 | −27.555 | 1.00 | 14.97 | A | C |
| ATOM | 1782 | CG | GLN | A | 234 | 11.303 | −45.270 | −28.993 | 1.00 | 14.65 | A | C |
| ATOM | 1783 | CD | GLN | A | 234 | 10.815 | −46.672 | −29.269 | 1.00 | 14.44 | A | C |
| ATOM | 1784 | OE1 | GLN | A | 234 | 9.965 | −47.219 | −28.560 | 1.00 | 14.43 | A | O |
| ATOM | 1785 | NE2 | GLN | A | 234 | 11.323 | −47.247 | −30.327 | 1.00 | 14.09 | A | N |
| ATOM | 1786 | C | GLN | A | 234 | 14.139 | −45.538 | −27.894 | 1.00 | 15.79 | A | C |
| ATOM | 1787 | O | GLN | A | 234 | 14.349 | −46.002 | −29.017 | 1.00 | 14.62 | A | O |
| ATOM | 1788 | N | GLY | A | 235 | 14.970 | −44.664 | −27.344 | 1.00 | 16.03 | A | N |
| ATOM | 1789 | CA | GLY | A | 235 | 16.065 | −44.118 | −28.151 | 1.00 | 16.48 | A | C |
| ATOM | 1790 | C | GLY | A | 235 | 15.602 | −43.077 | −29.169 | 1.00 | 16.81 | A | C |
| ATOM | 1791 | O | GLY | A | 235 | 14.511 | −43.182 | −29.751 | 1.00 | 17.76 | A | O |
| ATOM | 1792 | N | GLY | A | 236 | 16.422 | −42.048 | −29.351 | 1.00 | 16.10 | A | N |
| ATOM | 1793 | CA | GLY | A | 236 | 16.220 | −41.069 | −30.403 | 1.00 | 15.98 | A | C |
| ATOM | 1794 | C | GLY | A | 236 | 17.318 | −40.016 | −30.319 | 1.00 | 16.20 | A | C |
| ATOM | 1795 | O | GLY | A | 236 | 18.156 | −40.076 | −29.432 | 1.00 | 17.05 | A | O |
| ATOM | 1796 | N | THR | A | 237 | 17.335 | −39.061 | −31.239 | 1.00 | 15.70 | A | N |
| ATOM | 1797 | CA | THR | A | 237 | 18.164 | −37.893 | −31.078 | 1.00 | 15.66 | A | C |
| ATOM | 1798 | CB | THR | A | 237 | 19.028 | −37.680 | −32.316 | 1.00 | 15.65 | A | C |
| ATOM | 1799 | OG1 | THR | A | 237 | 20.044 | −38.688 | −32.371 | 1.00 | 15.12 | A | O |
| ATOM | 1800 | CG2 | THR | A | 237 | 19.671 | −36.339 | −32.258 | 1.00 | 15.82 | A | C |
| ATOM | 1801 | C | THR | A | 237 | 17.280 | −36.671 | −30.870 | 1.00 | 16.10 | A | C |
| ATOM | 1802 | O | THR | A | 237 | 16.260 | −36.549 | −31.531 | 1.00 | 17.31 | A | O |
| ATOM | 1803 | N | HIS | A | 238 | 17.707 | −35.747 | −30.000 | 1.00 | 16.14 | A | N |
| ATOM | 1804 | CA | HIS | A | 238 | 16.896 | −34.631 | −29.507 | 1.00 | 15.59 | A | C |
| ATOM | 1805 | CB | HIS | A | 238 | 16.100 | −35.136 | −28.320 | 1.00 | 15.65 | A | C |
| ATOM | 1806 | CG | HIS | A | 238 | 15.015 | −34.220 | −27.870 | 1.00 | 15.62 | A | C |
| ATOM | 1807 | ND1 | HIS | A | 238 | 15.038 | −33.597 | −26.642 | 1.00 | 15.28 | A | N |
| ATOM | 1808 | CE1 | HIS | A | 238 | 13.957 | −32.857 | −26.511 | 1.00 | 15.61 | A | C |
| ATOM | 1809 | NE2 | HIS | A | 238 | 13.239 | −32.969 | −27.616 | 1.00 | 15.96 | A | N |
| ATOM | 1810 | CD2 | HIS | A | 238 | 13.874 | −33.828 | −28.478 | 1.00 | 15.56 | A | C |
| ATOM | 1811 | C | HIS | A | 238 | 17.811 | −33.486 | −29.054 | 1.00 | 16.36 | A | C |
| ATOM | 1812 | O | HIS | A | 238 | 18.767 | −33.708 | −28.308 | 1.00 | 16.31 | A | O |
| ATOM | 1813 | N | TYR | A | 239 | 17.509 | −32.261 | −29.481 | 1.00 | 17.28 | A | N |
| ATOM | 1814 | CA | TYR | A | 239 | 18.437 | −31.129 | −29.358 | 1.00 | 18.38 | A | C |
| ATOM | 1815 | CB | TYR | A | 239 | 18.194 | −30.334 | −28.055 | 1.00 | 18.88 | A | C |
| ATOM | 1816 | CG | TYR | A | 239 | 16.844 | −29.703 | −28.089 | 1.00 | 19.54 | A | C |
| ATOM | 1817 | CD1 | TYR | A | 239 | 16.565 | −28.661 | −28.966 | 1.00 | 19.99 | A | C |
| ATOM | 1818 | CE1 | TYR | A | 239 | 15.286 | −28.100 | −29.034 | 1.00 | 21.13 | A | C |
| ATOM | 1819 | CZ | TYR | A | 239 | 14.263 | −28.596 | −28.214 | 1.00 | 21.56 | A | C |
| ATOM | 1820 | OH | TYR | A | 239 | 12.986 | −28.064 | −28.260 | 1.00 | 23.19 | A | O |
| ATOM | 1821 | CE2 | TYR | A | 239 | 14.530 | −29.623 | −27.329 | 1.00 | 20.93 | A | C |
| ATOM | 1822 | CD2 | TYR | A | 239 | 15.809 | −30.183 | −27.281 | 1.00 | 20.58 | A | C |
| ATOM | 1823 | C | TYR | A | 239 | 19.906 | −31.552 | −29.542 | 1.00 | 18.22 | A | C |
| ATOM | 1824 | O | TYR | A | 239 | 20.776 | −31.289 | −28.713 | 1.00 | 18.56 | A | O |
| ATOM | 1825 | N | GLY | A | 240 | 20.155 | −32.254 | −30.633 | 1.00 | 18.16 | A | N |
| ATOM | 1826 | CA | GLY | A | 240 | 21.496 | −32.645 | −30.989 | 1.00 | 17.75 | A | C |
| ATOM | 1827 | C | GLY | A | 240 | 22.090 | −33.814 | −30.251 | 1.00 | 17.52 | A | C |
| ATOM | 1828 | O | GLY | A | 240 | 23.170 | −34.223 | −30.605 | 1.00 | 19.02 | A | O |
| ATOM | 1829 | N | VAL | A | 241 | 21.407 | −34.384 | −29.263 | 1.00 | 17.08 | A | N |
| ATOM | 1830 | CA | VAL | A | 241 | 22.001 | −35.435 | −28.424 | 1.00 | 16.66 | A | C |
| ATOM | 1831 | CB | VAL | A | 241 | 21.800 | −35.134 | −26.929 | 1.00 | 16.31 | A | C |
| ATOM | 1832 | CG1 | VAL | A | 241 | 22.300 | −36.290 | −26.063 | 1.00 | 15.93 | A | C |
| ATOM | 1833 | CG2 | VAL | A | 241 | 22.477 | −33.828 | −26.550 | 1.00 | 16.07 | A | C |
| ATOM | 1834 | C | VAL | A | 241 | 21.381 | −36.786 | −28.715 | 1.00 | 17.12 | A | C |
| ATOM | 1835 | O | VAL | A | 241 | 20.177 | −36.895 | −28.798 | 1.00 | 17.62 | A | O |
| ATOM | 1836 | N | THR | A | 242 | 22.204 | −37.820 | −28.817 | 1.00 | 17.43 | A | N |
| ATOM | 1837 | CA | THR | A | 242 | 21.744 | −39.121 | −29.280 | 1.00 | 18.51 | A | C |
| ATOM | 1838 | CB | THR | A | 242 | 22.672 | −39.649 | −30.391 | 1.00 | 18.77 | A | C |
| ATOM | 1839 | OG1 | THR | A | 242 | 22.716 | −38.675 | −31.456 | 1.00 | 20.65 | A | O |
| ATOM | 1840 | CG2 | THR | A | 242 | 22.198 | −40.996 | −30.941 | 1.00 | 17.75 | A | C |
| ATOM | 1841 | C | THR | A | 242 | 21.696 | −40.102 | −28.124 | 1.00 | 19.15 | A | C |
| ATOM | 1842 | O | THR | A | 242 | 22.645 | −40.197 | −27.349 | 1.00 | 20.63 | A | O |
| ATOM | 1843 | N | VAL | A | 243 | 20.597 | −40.841 | −28.008 | 1.00 | 18.62 | A | N |
| ATOM | 1844 | CA | VAL | A | 243 | 20.381 | −41.700 | −26.852 | 1.00 | 17.91 | A | C |
| ATOM | 1845 | CB | VAL | A | 243 | 19.219 | −41.203 | −25.937 | 1.00 | 17.53 | A | C |
| ATOM | 1846 | CG1 | VAL | A | 243 | 18.882 | −42.211 | −24.866 | 1.00 | 17.59 | A | C |
| ATOM | 1847 | CG2 | VAL | A | 243 | 19.540 | −39.887 | −25.267 | 1.00 | 17.36 | A | C |
| ATOM | 1848 | C | VAL | A | 243 | 20.045 | −43.047 | −27.402 | 1.00 | 17.83 | A | C |
| ATOM | 1849 | O | VAL | A | 243 | 19.182 | −43.165 | −28.255 | 1.00 | 17.31 | A | O |
| ATOM | 1850 | N | ASN | A | 244 | 20.728 | −44.064 | −26.896 | 1.00 | 18.98 | A | N |
| ATOM | 1851 | CA | ASN | A | 244 | 20.444 | −45.429 | −27.283 | 1.00 | 19.14 | A | C |
| ATOM | 1852 | CB | ASN | A | 244 | 21.743 | −46.233 | −27.435 | 1.00 | 20.07 | A | C |
| ATOM | 1853 | CG | ASN | A | 244 | 22.687 | −45.637 | −28.492 | 1.00 | 22.13 | A | C |
| ATOM | 1854 | OD1 | ASN | A | 244 | 23.898 | −45.673 | −28.328 | 1.00 | 25.76 | A | O |
| ATOM | 1855 | ND2 | ASN | A | 244 | 22.139 | −45.076 | −29.571 | 1.00 | 22.37 | A | N |
| ATOM | 1856 | C | ASN | A | 244 | 19.487 | −46.073 | −26.293 | 1.00 | 17.74 | A | C |
| ATOM | 1857 | O | ASN | A | 244 | 19.867 | −46.368 | −25.167 | 1.00 | 17.39 | A | O |
| ATOM | 1858 | N | GLY | A | 245 | 18.250 | −46.288 | −26.756 | 1.00 | 16.76 | A | N |
| ATOM | 1859 | CA | GLY | A | 245 | 17.168 | −46.961 | −26.003 | 1.00 | 15.81 | A | C |
| ATOM | 1860 | C | GLY | A | 245 | 17.533 | −48.262 | −25.293 | 1.00 | 14.82 | A | C |
| ATOM | 1861 | O | GLY | A | 245 | 18.329 | −49.031 | −25.768 | 1.00 | 13.85 | A | O |
| ATOM | 1862 | N | ILE | A | 246 | 16.931 | −48.493 | −24.137 | 1.00 | 14.54 | A | N |
| ATOM | 1863 | CA | ILE | A | 246 | 17.202 | −49.690 | −23.331 | 1.00 | 14.23 | A | C |
| ATOM | 1864 | CB | ILE | A | 246 | 17.692 | −49.293 | −21.907 | 1.00 | 14.22 | A | C |
| ATOM | 1865 | CG1 | ILE | A | 246 | 16.529 | −48.766 | −21.060 | 1.00 | 13.97 | A | C |
| ATOM | 1866 | CD1 | ILE | A | 246 | 16.857 | −48.508 | −19.623 | 1.00 | 14.08 | A | C |
| ATOM | 1867 | CG2 | ILE | A | 246 | 18.784 | −48.218 | −22.012 | 1.00 | 14.32 | A | C |
| ATOM | 1868 | C | ILE | A | 246 | 15.965 | −50.552 | −23.209 | 1.00 | 13.73 | A | C |
| ATOM | 1869 | O | ILE | A | 246 | 15.987 | −51.557 | −22.540 | 1.00 | 12.83 | A | O |
| ATOM | 1870 | N | GLY | A | 247 | 14.880 | −50.097 | −23.820 | 1.00 | 14.78 | A | N |
| ATOM | 1871 | CA | GLY | A | 247 | 13.606 | −50.812 | −23.849 | 1.00 | 15.96 | A | C |
| ATOM | 1872 | C | GLY | A | 247 | 12.624 | −50.351 | −22.790 | 1.00 | 17.03 | A | C |
| ATOM | 1873 | O | GLY | A | 247 | 12.999 | −49.794 | −21.732 | 1.00 | 17.49 | A | O |
| ATOM | 1874 | N | ARG | A | 248 | 11.349 | −50.581 | −23.066 | 1.00 | 17.34 | A | N |
| ATOM | 1875 | CA | ARG | A | 248 | 10.309 | −50.188 | −22.118 | 1.00 | 17.10 | A | C |
| ATOM | 1876 | CB | ARG | A | 248 | 8.940 | −50.518 | −22.669 | 1.00 | 16.58 | A | C |
| ATOM | 1877 | CG | ARG | A | 248 | 8.433 | −49.504 | −23.662 | 1.00 | 16.47 | A | C |
| ATOM | 1878 | CD | ARG | A | 248 | 7.050 | −49.883 | −24.118 | 1.00 | 16.25 | A | C |
| ATOM | 1879 | NE | ARG | A | 248 | 6.539 | −48.977 | −25.139 | 1.00 | 17.32 | A | N |
| ATOM | 1880 | CZ | ARG | A | 248 | 5.613 | −48.024 | −24.967 | 1.00 | 18.41 | A | C |
| ATOM | 1881 | NH1 | ARG | A | 248 | 5.058 | −47.761 | −23.787 | 1.00 | 18.84 | A | N |
| ATOM | 1882 | NH2 | ARG | A | 248 | 5.232 | −47.308 | −26.009 | 1.00 | 18.99 | A | N |
| ATOM | 1883 | C | ARG | A | 248 | 10.493 | −50.875 | −20.763 | 1.00 | 17.84 | A | C |
| ATOM | 1884 | O | ARG | A | 248 | 10.415 | −50.214 | −19.716 | 1.00 | 18.39 | A | O |
| ATOM | 1885 | N | ASP | A | 249 | 10.747 | −52.182 | −20.760 | 1.00 | 17.69 | A | N |
| ATOM | 1886 | CA | ASP | A | 249 | 10.706 | −52.918 | −19.487 | 1.00 | 17.91 | A | C |
| ATOM | 1887 | CB | ASP | A | 249 | 10.937 | −54.421 | −19.668 | 1.00 | 19.68 | A | C |
| ATOM | 1888 | CG | ASP | A | 249 | 9.848 | −55.096 | −20.488 | 1.00 | 21.62 | A | C |
| ATOM | 1889 | OD1 | ASP | A | 249 | 8.623 | −54.884 | −20.223 | 1.00 | 23.50 | A | O |
| ATOM | 1890 | OD2 | ASP | A | 249 | 10.246 | −55.848 | −21.401 | 1.00 | 22.32 | A | O |
| ATOM | 1891 | C | ASP | A | 249 | 11.718 | −52.389 | −18.500 | 1.00 | 15.89 | A | C |
| ATOM | 1892 | O | ASP | A | 249 | 11.395 | −52.143 | −17.365 | 1.00 | 15.97 | A | O |
| ATOM | 1893 | N | ALA | A | 250 | 12.943 | −52.202 | −18.940 | 1.00 | 14.48 | A | N |
| ATOM | 1894 | CA | ALA | A | 250 | 13.986 | −51.761 | −18.044 | 1.00 | 13.79 | A | C |
| ATOM | 1895 | CB | ALA | A | 250 | 15.331 | −51.950 | −18.717 | 1.00 | 13.75 | A | C |
| ATOM | 1896 | C | ALA | A | 250 | 13.806 | −50.299 | −17.587 | 1.00 | 13.38 | A | C |
| ATOM | 1897 | O | ALA | A | 250 | 14.111 | −49.946 | −16.455 | 1.00 | 12.91 | A | O |
| ATOM | 1898 | N | ALA | A | 251 | 13.325 | −49.444 | −18.474 | 1.00 | 13.31 | A | N |
| ATOM | 1899 | CA | ALA | A | 251 | 13.146 | −48.054 | −18.117 | 1.00 | 13.55 | A | C |
| ATOM | 1900 | CB | ALA | A | 251 | 12.906 | −47.192 | −19.342 | 1.00 | 13.75 | A | C |
| ATOM | 1901 | C | ALA | A | 251 | 12.032 | −47.853 | −17.090 | 1.00 | 13.33 | A | C |
| ATOM | 1902 | O | ALA | A | 251 | 12.242 | −47.100 | −16.146 | 1.00 | 13.54 | A | O |
| ATOM | 1903 | N | VAL | A | 252 | 10.872 | −48.499 | −17.238 | 1.00 | 13.18 | A | N |
| ATOM | 1904 | CA | VAL | A | 252 | 9.833 | −48.337 | −16.191 | 1.00 | 13.49 | A | C |
| ATOM | 1905 | CB | VAL | A | 252 | 8.454 | −49.008 | −16.461 | 1.00 | 13.62 | A | C |
| ATOM | 1906 | CG2 | VAL | A | 252 | 8.450 | −50.435 | −15.961 | 1.00 | 14.26 | A | C |
| ATOM | 1907 | CG1 | VAL | A | 252 | 8.094 | −49.017 | −17.928 | 1.00 | 13.79 | A | C |
| ATOM | 1908 | C | VAL | A | 252 | 10.377 | −48.832 | −14.850 | 1.00 | 13.05 | A | C |
| ATOM | 1909 | O | VAL | A | 252 | 10.070 | −48.244 | −13.836 | 1.00 | 12.86 | A | O |
| ATOM | 1910 | N | GLN | A | 253 | 11.193 | −49.886 | −14.855 | 1.00 | 13.17 | A | N |
| ATOM | 1911 | CA | GLN | A | 253 | 11.744 | −50.453 | −13.619 | 1.00 | 13.59 | A | C |
| ATOM | 1912 | CB | GLN | A | 253 | 12.576 | −51.689 | −13.924 | 1.00 | 14.23 | A | C |
| ATOM | 1913 | CG | GLN | A | 253 | 12.479 | −52.726 | −12.823 | 1.00 | 15.43 | A | C |
| ATOM | 1914 | CD | GLN | A | 253 | 11.152 | −53.484 | −12.862 | 1.00 | 16.19 | A | C |
| ATOM | 1915 | OE1 | GLN | A | 253 | 10.488 | −53.660 | −11.849 | 1.00 | 16.30 | A | O |
| ATOM | 1916 | NE2 | GLN | A | 253 | 10.760 | −53.921 | −14.052 | 1.00 | 16.56 | A | N |
| ATOM | 1917 | C | GLN | A | 253 | 12.614 | −49.427 | −12.890 | 1.00 | 13.62 | A | C |
| ATOM | 1918 | O | GLN | A | 253 | 12.418 | −49.127 | −11.705 | 1.00 | 13.49 | A | O |
| ATOM | 1919 | N | ILE | A | 254 | 13.559 | −48.862 | −13.633 | 1.00 | 13.32 | A | N |
| ATOM | 1920 | CA | ILE | A | 254 | 14.372 | −47.776 | −13.145 | 1.00 | 13.03 | A | C |
| ATOM | 1921 | CB | ILE | A | 254 | 15.269 | −47.254 | −14.264 | 1.00 | 13.19 | A | C |
| ATOM | 1922 | CG1 | ILE | A | 254 | 16.277 | −48.338 | −14.647 | 1.00 | 13.74 | A | C |
| ATOM | 1923 | CD1 | ILE | A | 254 | 17.007 | −48.092 | −15.967 | 1.00 | 13.85 | A | C |
| ATOM | 1924 | CG2 | ILE | A | 254 | 15.985 | −45.984 | −13.832 | 1.00 | 13.13 | A | C |
| ATOM | 1925 | C | ILE | A | 254 | 13.521 | −46.622 | −12.589 | 1.00 | 12.90 | A | C |
| ATOM | 1926 | O | ILE | A | 254 | 13.843 | −46.043 | −11.541 | 1.00 | 12.83 | A | O |
| ATOM | 1927 | N | PHE | A | 255 | 12.453 | −46.249 | −13.273 | 1.00 | 12.49 | A | N |
| ATOM | 1928 | CA | PHE | A | 255 | 11.664 | −45.128 | −12.753 | 1.00 | 12.76 | A | C |
| ATOM | 1929 | CB | PHE | A | 255 | 10.822 | −44.475 | −13.840 | 1.00 | 12.83 | A | C |
| ATOM | 1930 | CG | PHE | A | 255 | 11.582 | −43.498 | −14.655 | 1.00 | 12.70 | A | C |
| ATOM | 1931 | CD1 | PHE | A | 255 | 12.530 | −43.932 | −15.557 | 1.00 | 12.83 | A | C |
| ATOM | 1932 | CE1 | PHE | A | 255 | 13.259 | −43.028 | −16.307 | 1.00 | 12.62 | A | C |
| ATOM | 1933 | CZ | PHE | A | 255 | 13.044 | −41.683 | −16.153 | 1.00 | 12.29 | A | C |
| ATOM | 1934 | CE2 | PHE | A | 255 | 12.105 | −41.249 | −15.251 | 1.00 | 12.48 | A | C |
| ATOM | 1935 | CD2 | PHE | A | 255 | 11.384 | −42.153 | −14.498 | 1.00 | 12.59 | A | C |
| ATOM | 1936 | C | PHE | A | 255 | 10.813 | −45.495 | −11.534 | 1.00 | 12.72 | A | C |
| ATOM | 1937 | O | PHE | A | 255 | 10.656 | −44.670 | −10.623 | 1.00 | 12.55 | A | O |
| ATOM | 1938 | N | TYR | A | 256 | 10.308 | −46.730 | −11.494 | 1.00 | 12.65 | A | N |
| ATOM | 1939 | CA | TYR | A | 256 | 9.621 | −47.217 | −10.288 | 1.00 | 12.85 | A | C |
| ATOM | 1940 | CB | TYR | A | 256 | 8.944 | −48.575 | −10.520 | 1.00 | 12.32 | A | C |
| ATOM | 1941 | CG | TYR | A | 256 | 8.231 | −49.150 | −9.321 | 1.00 | 12.10 | A | C |
| ATOM | 1942 | CD1 | TYR | A | 256 | 6.940 | −48.766 | −8.987 | 1.00 | 11.97 | A | C |
| ATOM | 1943 | CE1 | TYR | A | 256 | 6.291 | −49.324 | −7.886 | 1.00 | 12.35 | A | C |
| ATOM | 1944 | CZ | TYR | A | 256 | 6.958 | −50.305 | −7.087 | 1.00 | 12.71 | A | C |
| ATOM | 1945 | OH | TYR | A | 256 | 6.407 | −50.893 | −5.963 | 1.00 | 11.74 | A | O |
| ATOM | 1946 | CE2 | TYR | A | 256 | 8.240 | −50.693 | −7.431 | 1.00 | 12.63 | A | C |
| ATOM | 1947 | CD2 | TYR | A | 256 | 8.857 | −50.119 | −8.537 | 1.00 | 12.42 | A | C |
| ATOM | 1948 | C | TYR | A | 256 | 10.606 | −47.259 | −9.105 | 1.00 | 13.23 | A | C |
| ATOM | 1949 | O | TYR | A | 256 | 10.259 | −46.767 | −8.003 | 1.00 | 13.67 | A | O |
| ATOM | 1950 | N | ASP | A | 257 | 11.821 | −47.782 | −9.328 | 1.00 | 12.80 | A | N |
| ATOM | 1951 | CA | ASP | A | 257 | 12.798 | −47.841 | −8.248 | 1.00 | 12.91 | A | C |
| ATOM | 1952 | CB | ASP | A | 257 | 14.042 | −48.604 | −8.654 | 1.00 | 13.70 | A | C |
| ATOM | 1953 | CG | ASP | A | 257 | 13.783 | −50.108 | −8.772 | 1.00 | 14.87 | A | C |
| ATOM | 1954 | OD1 | ASP | A | 257 | 12.705 | −50.623 | −8.313 | 1.00 | 14.92 | A | O |
| ATOM | 1955 | OD2 | ASP | A | 257 | 14.671 | −50.780 | −9.354 | 1.00 | 16.53 | A | O |
| ATOM | 1956 | C | ASP | A | 257 | 13.162 | −46.462 | −7.742 | 1.00 | 12.44 | A | C |
| ATOM | 1957 | O | ASP | A | 257 | 13.202 | −46.234 | −6.512 | 1.00 | 11.99 | A | O |
| ATOM | 1958 | N | ALA | A | 258 | 13.393 | −45.540 | −8.675 | 1.00 | 11.72 | A | N |
| ATOM | 1959 | CA | ALA | A | 258 | 13.632 | −44.133 | −8.324 | 1.00 | 11.33 | A | C |
| ATOM | 1960 | CB | ALA | A | 258 | 13.821 | −43.289 | −9.581 | 1.00 | 11.03 | A | C |
| ATOM | 1961 | C | ALA | A | 258 | 12.476 | −43.580 | −7.512 | 1.00 | 11.08 | A | C |
| ATOM | 1962 | O | ALA | A | 258 | 12.650 | −42.782 | −6.597 | 1.00 | 10.58 | A | O |
| ATOM | 1963 | N | LEU | A | 259 | 11.274 | −43.985 | −7.885 | 1.00 | 11.33 | A | N |
| ATOM | 1964 | CA | LEU | A | 259 | 10.070 | −43.427 | −7.265 | 1.00 | 11.50 | A | C |
| ATOM | 1965 | CB | LEU | A | 259 | 8.813 | −43.839 | −8.048 | 1.00 | 11.12 | A | C |
| ATOM | 1966 | CG | LEU | A | 259 | 7.498 | −43.498 | −7.383 | 1.00 | 11.01 | A | C |
| ATOM | 1967 | CD1 | LEU | A | 259 | 7.281 | −41.998 | −7.298 | 1.00 | 11.10 | A | C |
| ATOM | 1968 | CD2 | LEU | A | 259 | 6.381 | −44.152 | −8.163 | 1.00 | 11.25 | A | C |
| ATOM | 1969 | C | LEU | A | 259 | 9.974 | −43.858 | −5.809 | 1.00 | 11.65 | A | C |
| ATOM | 1970 | O | LEU | A | 259 | 9.729 | −43.042 | −4.933 | 1.00 | 11.15 | A | O |
| ATOM | 1971 | N | ILE | A | 260 | 10.190 | −45.143 | −5.559 | 1.00 | 12.27 | A | N |
| ATOM | 1972 | CA | ILE | A | 260 | 9.993 | −45.655 | −4.222 | 1.00 | 13.14 | A | C |
| ATOM | 1973 | CB | ILE | A | 260 | 9.454 | −47.117 | −4.241 | 1.00 | 13.43 | A | C |
| ATOM | 1974 | CG1 | ILE | A | 260 | 10.522 | −48.112 | −4.719 | 1.00 | 13.50 | A | C |
| ATOM | 1975 | CD1 | ILE | A | 260 | 10.234 | −49.531 | −4.306 | 1.00 | 13.29 | A | C |
| ATOM | 1976 | CG2 | ILE | A | 260 | 8.188 | −47.221 | −5.092 | 1.00 | 13.46 | A | C |
| ATOM | 1977 | C | ILE | A | 260 | 11.215 | −45.571 | −3.302 | 1.00 | 13.39 | A | C |
| ATOM | 1978 | O | ILE | A | 260 | 11.093 | −45.924 | −2.160 | 1.00 | 14.38 | A | O |
| ATOM | 1979 | N | ASN | A | 261 | 12.376 | −45.141 | −3.776 | 1.00 | 13.69 | A | N |
| ATOM | 1980 | CA | ASN | A | 261 | 13.594 | −45.072 | −2.958 | 1.00 | 13.71 | A | C |
| ATOM | 1981 | CB | ASN | A | 261 | 14.671 | −46.031 | −3.498 | 1.00 | 13.86 | A | C |
| ATOM | 1982 | CG | ASN | A | 261 | 14.287 | −47.512 | −3.332 | 1.00 | 14.28 | A | C |
| ATOM | 1983 | OD1 | ASN | A | 261 | 13.711 | −47.912 | −2.344 | 1.00 | 14.95 | A | O |
| ATOM | 1984 | ND2 | ASN | A | 261 | 14.606 | −48.317 | −4.304 | 1.00 | 14.25 | A | N |
| ATOM | 1985 | C | ASN | A | 261 | 14.149 | −43.646 | −2.898 | 1.00 | 14.22 | A | C |
| ATOM | 1986 | O | ASN | A | 261 | 14.355 | −43.085 | −1.825 | 1.00 | 14.32 | A | O |
| ATOM | 1987 | N | TYR | A | 262 | 14.379 | −43.034 | −4.044 | 1.00 | 14.90 | A | N |
| ATOM | 1988 | CA | TYR | A | 262 | 15.196 | −41.827 | −4.071 | 1.00 | 15.53 | A | C |
| ATOM | 1989 | CB | TYR | A | 262 | 16.323 | −41.998 | −5.092 | 1.00 | 15.60 | A | C |
| ATOM | 1990 | CG | TYR | A | 262 | 17.157 | −43.252 | −4.863 | 1.00 | 15.76 | A | C |
| ATOM | 1991 | CD1 | TYR | A | 262 | 17.756 | −43.490 | −3.638 | 1.00 | 15.71 | A | C |
| ATOM | 1992 | CE1 | TYR | A | 262 | 18.511 | −44.630 | −3.413 | 1.00 | 15.96 | A | C |
| ATOM | 1993 | CZ | TYR | A | 262 | 18.692 | −45.572 | −4.420 | 1.00 | 15.60 | A | C |
| ATOM | 1994 | OH | TYR | A | 262 | 19.453 | −46.702 | −4.174 | 1.00 | 13.73 | A | O |
| ATOM | 1995 | CE2 | TYR | A | 262 | 18.097 | −45.353 | −5.655 | 1.00 | 16.02 | A | C |
| ATOM | 1996 | CD2 | TYR | A | 262 | 17.341 | −44.199 | −5.872 | 1.00 | 15.99 | A | C |
| ATOM | 1997 | C | TYR | A | 262 | 14.422 | −40.520 | −4.293 | 1.00 | 15.92 | A | C |
| ATOM | 1998 | O | TYR | A | 262 | 14.850 | −39.469 | −3.830 | 1.00 | 17.27 | A | O |
| ATOM | 1999 | N | LEU | A | 263 | 13.284 | −40.543 | −4.957 | 1.00 | 15.38 | A | N |
| ATOM | 2000 | CA | LEU | A | 263 | 12.508 | −39.307 | −5.019 | 1.00 | 15.76 | A | C |
| ATOM | 2001 | CB | LEU | A | 263 | 11.362 | −39.431 | −6.036 | 1.00 | 16.59 | A | C |
| ATOM | 2002 | CG | LEU | A | 263 | 11.562 | −38.897 | −7.463 | 1.00 | 16.71 | A | C |
| ATOM | 2003 | CD1 | LEU | A | 263 | 13.023 | −38.786 | −7.862 | 1.00 | 16.41 | A | C |
| ATOM | 2004 | CD2 | LEU | A | 263 | 10.766 | −39.742 | −8.454 | 1.00 | 16.56 | A | C |
| ATOM | 2005 | C | LEU | A | 263 | 11.949 | −38.939 | −3.637 | 1.00 | 15.00 | A | C |
| ATOM | 2006 | O | LEU | A | 263 | 11.773 | −39.803 | −2.785 | 1.00 | 15.82 | A | O |
| ATOM | 2007 | N | THR | A | 264 | 11.680 | −37.654 | −3.439 | 1.00 | 14.04 | A | N |
| ATOM | 2008 | CA | THR | A | 264 | 11.150 | −37.105 | −2.182 | 1.00 | 13.18 | A | C |
| ATOM | 2009 | CB | THR | A | 264 | 12.254 | −36.395 | −1.347 | 1.00 | 12.53 | A | C |
| ATOM | 2010 | OG1 | THR | A | 264 | 12.519 | −35.080 | −1.876 | 1.00 | 12.08 | A | O |
| ATOM | 2011 | CG2 | THR | A | 264 | 13.522 | −37.213 | −1.294 | 1.00 | 12.33 | A | C |
| ATOM | 2012 | C | THR | A | 264 | 10.061 | −36.059 | −2.490 | 1.00 | 13.48 | A | C |
| ATOM | 2013 | O | THR | A | 264 | 9.862 | −35.671 | −3.655 | 1.00 | 13.25 | A | O |
| ATOM | 2014 | N | PRO | A | 265 | 9.396 | −35.535 | −1.446 | 1.00 | 13.46 | A | N |
| ATOM | 2015 | CA | PRO | A | 265 | 8.244 | −34.703 | −1.688 | 1.00 | 13.44 | A | C |
| ATOM | 2016 | CB | PRO | A | 265 | 7.713 | −34.453 | −0.278 | 1.00 | 13.36 | A | C |
| ATOM | 2017 | CG | PRO | A | 265 | 8.105 | −35.667 | 0.469 | 1.00 | 13.47 | A | C |
| ATOM | 2018 | CD | PRO | A | 265 | 9.514 | −35.838 | −0.014 | 1.00 | 13.66 | A | C |
| ATOM | 2019 | C | PRO | A | 265 | 8.584 | −33.392 | −2.320 | 1.00 | 13.55 | A | C |
| ATOM | 2020 | O | PRO | A | 265 | 7.713 | −32.600 | −2.615 | 1.00 | 14.25 | A | O |
| ATOM | 2021 | N | THR | A | 266 | 9.862 | −33.187 | −2.529 | 1.00 | 13.50 | A | N |
| ATOM | 2022 | CA | THR | A | 266 | 10.439 | −31.881 | −2.743 | 1.00 | 12.66 | A | C |
| ATOM | 2023 | CB | THR | A | 266 | 11.222 | −31.577 | −1.437 | 1.00 | 12.59 | A | C |
| ATOM | 2024 | OG1 | THR | A | 266 | 10.606 | −30.478 | −0.813 | 1.00 | 12.07 | A | O |
| ATOM | 2025 | CG2 | THR | A | 266 | 12.736 | −31.331 | −1.610 | 1.00 | 12.62 | A | C |
| ATOM | 2026 | C | THR | A | 266 | 11.320 | −31.926 | −3.951 | 1.00 | 12.14 | A | C |
| ATOM | 2027 | O | THR | A | 266 | 11.955 | −30.972 | −4.264 | 1.00 | 12.11 | A | O |
| ATOM | 2028 | N | SER | A | 267 | 11.378 | −33.074 | −4.611 | 1.00 | 12.40 | A | N |
| ATOM | 2029 | CA | SER | A | 267 | 12.317 | −33.305 | −5.672 | 1.00 | 12.42 | A | C |
| ATOM | 2030 | CB | SER | A | 267 | 12.291 | −34.755 | −6.099 | 1.00 | 12.13 | A | C |
| ATOM | 2031 | OG | SER | A | 267 | 12.643 | −35.580 | −5.035 | 1.00 | 11.99 | A | O |
| ATOM | 2032 | C | SER | A | 267 | 11.968 | −32.465 | −6.859 | 1.00 | 13.24 | A | C |
| ATOM | 2033 | O | SER | A | 267 | 10.812 | −32.409 | −7.303 | 1.00 | 13.97 | A | O |
| ATOM | 2034 | N | ASN | A | 268 | 12.978 | −31.795 | −7.366 | 1.00 | 13.85 | A | N |
| ATOM | 2035 | CA | ASN | A | 268 | 12.878 | −31.150 | −8.667 | 1.00 | 14.49 | A | C |
| ATOM | 2036 | CB | ASN | A | 268 | 13.528 | −29.763 | −8.601 | 1.00 | 14.22 | A | C |
| ATOM | 2037 | CG | ASN | A | 268 | 15.016 | −29.822 | −8.253 | 1.00 | 13.98 | A | C |
| ATOM | 2038 | OD1 | ASN | A | 268 | 15.707 | −30.838 | −8.451 | 1.00 | 13.99 | A | O |
| ATOM | 2039 | ND2 | ASN | A | 268 | 15.511 | −28.726 | −7.730 | 1.00 | 13.52 | A | N |
| ATOM | 2040 | C | ASN | A | 268 | 13.532 | −31.987 | −9.787 | 1.00 | 15.25 | A | C |
| ATOM | 2041 | O | ASN | A | 268 | 14.066 | −33.113 | −9.552 | 1.00 | 14.36 | A | O |
| ATOM | 2042 | N | PHE | A | 269 | 13.499 | −31.408 | −10.994 | 1.00 | 15.91 | A | N |
| ATOM | 2043 | CA | PHE | A | 269 | 13.978 | −32.076 | −12.198 | 1.00 | 16.68 | A | C |
| ATOM | 2044 | CB | PHE | A | 269 | 13.906 | −31.122 | −13.401 | 1.00 | 16.90 | A | C |
| ATOM | 2045 | CG | PHE | A | 269 | 12.562 | −31.075 | −14.062 | 1.00 | 16.51 | A | C |
| ATOM | 2046 | CD2 | PHE | A | 269 | 11.620 | −30.146 | −13.683 | 1.00 | 16.34 | A | C |
| ATOM | 2047 | CE2 | PHE | A | 269 | 10.379 | −30.116 | −14.305 | 1.00 | 16.36 | A | C |
| ATOM | 2048 | CZ | PHE | A | 269 | 10.067 | −31.015 | −15.318 | 1.00 | 15.85 | A | C |
| ATOM | 2049 | CE1 | PHE | A | 269 | 10.985 | −31.944 | −15.704 | 1.00 | 15.68 | A | C |
| ATOM | 2050 | CD1 | PHE | A | 269 | 12.236 | −31.971 | −15.078 | 1.00 | 16.47 | A | C |
| ATOM | 2051 | C | PHE | A | 269 | 15.412 | −32.571 | −12.004 | 1.00 | 16.65 | A | C |
| ATOM | 2052 | O | PHE | A | 269 | 15.734 | −33.714 | −12.297 | 1.00 | 16.47 | A | O |
| ATOM | 2053 | N | SER | A | 270 | 16.261 | −31.703 | −11.487 | 1.00 | 17.12 | A | N |
| ATOM | 2054 | CA | SER | A | 270 | 17.660 | −32.075 | −11.211 | 1.00 | 17.53 | A | C |
| ATOM | 2055 | CB | SER | A | 270 | 18.420 | −30.901 | −10.589 | 1.00 | 17.92 | A | C |
| ATOM | 2056 | OG | SER | A | 270 | 19.771 | −31.074 | −10.914 | 1.00 | 19.56 | A | O |
| ATOM | 2057 | C | SER | A | 270 | 17.776 | −33.298 | −10.309 | 1.00 | 16.25 | A | C |
| ATOM | 2058 | O | SER | A | 270 | 18.521 | −34.249 | −10.592 | 1.00 | 16.12 | A | O |
| ATOM | 2059 | N | ALA | A | 271 | 17.014 | −33.274 | −9.226 | 1.00 | 15.54 | A | N |
| ATOM | 2060 | CA | ALA | A | 271 | 16.898 | −34.441 | −8.354 | 1.00 | 14.92 | A | C |
| ATOM | 2061 | CB | ALA | A | 271 | 16.098 | −34.060 | −7.101 | 1.00 | 15.08 | A | C |
| ATOM | 2062 | C | ALA | A | 271 | 16.276 | −35.679 | −9.083 | 1.00 | 13.85 | A | C |
| ATOM | 2063 | O | ALA | A | 271 | 16.669 | −36.848 | −8.852 | 1.00 | 12.80 | A | O |
| ATOM | 2064 | N | MET | A | 272 | 15.314 | −35.433 | −9.965 | 1.00 | 13.07 | A | N |
| ATOM | 2065 | CA | MET | A | 272 | 14.781 | −36.544 | −10.730 | 1.00 | 13.25 | A | C |
| ATOM | 2066 | CB | MET | A | 272 | 13.622 | −36.100 | −11.628 | 1.00 | 13.09 | A | C |
| ATOM | 2067 | CG | MET | A | 272 | 13.165 | −37.128 | −12.682 | 1.00 | 12.47 | A | C |
| ATOM | 2068 | SD | MET | A | 272 | 12.018 | −38.351 | −12.062 | 1.00 | 11.85 | A | S |
| ATOM | 2069 | CE | MET | A | 272 | 12.986 | −39.846 | −11.892 | 1.00 | 11.70 | A | C |
| ATOM | 2070 | C | MET | A | 272 | 15.882 | −37.220 | −11.555 | 1.00 | 13.54 | A | C |
| ATOM | 2071 | O | MET | A | 272 | 15.903 | −38.452 | −11.661 | 1.00 | 13.35 | A | O |
| ATOM | 2072 | N | ARG | A | 273 | 16.773 | −36.405 | −12.127 | 1.00 | 13.96 | A | N |
| ATOM | 2073 | CA | ARG | A | 273 | 17.927 | −36.880 | −12.896 | 1.00 | 14.72 | A | C |
| ATOM | 2074 | CB | ARG | A | 273 | 18.732 | −35.678 | −13.439 | 1.00 | 16.09 | A | C |
| ATOM | 2075 | CG | ARG | A | 273 | 19.897 | −36.062 | −14.351 | 1.00 | 17.57 | A | C |
| ATOM | 2076 | CD | ARG | A | 273 | 20.932 | −34.937 | −14.559 | 1.00 | 19.14 | A | C |
| ATOM | 2077 | NE | ARG | A | 273 | 22.199 | −35.555 | −15.019 | 1.00 | 20.49 | A | N |
| ATOM | 2078 | CZ | ARG | A | 273 | 22.627 | −35.627 | −16.282 | 1.00 | 19.97 | A | C |
| ATOM | 2079 | NH1 | ARG | A | 273 | 21.946 | −35.082 | −17.277 | 1.00 | 21.14 | A | N |
| ATOM | 2080 | NH2 | ARG | A | 273 | 23.746 | −36.263 | −16.557 | 1.00 | 19.78 | A | N |
| ATOM | 2081 | C | ARG | A | 273 | 18.832 | −37.792 | −12.058 | 1.00 | 14.15 | A | C |
| ATOM | 2082 | O | ARG | A | 273 | 19.154 | −38.934 | −12.442 | 1.00 | 13.74 | A | O |
| ATOM | 2083 | N | ALA | A | 274 | 19.233 | −37.270 | −10.904 | 1.00 | 13.53 | A | N |
| ATOM | 2084 | CA | ALA | A | 274 | 20.050 | −38.025 | −9.967 | 1.00 | 12.90 | A | C |
| ATOM | 2085 | CB | ALA | A | 274 | 20.334 | −37.174 | −8.741 | 1.00 | 12.64 | A | C |
| ATOM | 2086 | C | ALA | A | 274 | 19.346 | −39.314 | −9.574 | 1.00 | 12.59 | A | C |
| ATOM | 2087 | O | ALA | A | 274 | 19.946 | −40.390 | −9.501 | 1.00 | 12.04 | A | O |
| ATOM | 2088 | N | ALA | A | 275 | 18.060 | −39.202 | −9.309 | 1.00 | 12.71 | A | N |
| ATOM | 2089 | CA | ALA | A | 275 | 17.357 | −40.339 | −8.776 | 1.00 | 13.34 | A | C |
| ATOM | 2090 | CB | ALA | A | 275 | 15.961 | −39.931 | −8.356 | 1.00 | 13.53 | A | C |
| ATOM | 2091 | C | ALA | A | 275 | 17.318 | −41.459 | −9.804 | 1.00 | 13.49 | A | C |
| ATOM | 2092 | O | ALA | A | 275 | 17.594 | −42.638 | −9.496 | 1.00 | 13.79 | A | O |
| ATOM | 2093 | N | ALA | A | 276 | 16.987 | −41.086 | −11.036 | 1.00 | 13.40 | A | N |
| ATOM | 2094 | CA | ALA | A | 276 | 16.968 | −42.041 | −12.114 | 1.00 | 13.48 | A | C |
| ATOM | 2095 | CB | ALA | A | 276 | 16.478 | −41.373 | −13.387 | 1.00 | 13.69 | A | C |
| ATOM | 2096 | C | ALA | A | 276 | 18.370 | −42.622 | −12.286 | 1.00 | 13.41 | A | C |
| ATOM | 2097 | O | ALA | A | 276 | 18.531 | −43.833 | −12.411 | 1.00 | 12.81 | A | O |
| ATOM | 2098 | N | ILE | A | 277 | 19.390 | −41.767 | −12.244 | 1.00 | 13.47 | A | N |
| ATOM | 2099 | CA | ILE | A | 277 | 20.744 | −42.282 | −12.313 | 1.00 | 13.96 | A | C |
| ATOM | 2100 | CB | ILE | A | 277 | 21.797 | −41.190 | −12.261 | 1.00 | 14.04 | A | C |
| ATOM | 2101 | CG1 | ILE | A | 277 | 21.814 | −40.438 | −13.581 | 1.00 | 14.13 | A | C |
| ATOM | 2102 | CD1 | ILE | A | 277 | 22.709 | −39.232 | −13.583 | 1.00 | 14.32 | A | C |
| ATOM | 2103 | CG2 | ILE | A | 277 | 23.163 | −41.829 | −12.023 | 1.00 | 14.29 | A | C |
| ATOM | 2104 | C | ILE | A | 277 | 21.066 | −43.276 | −11.210 | 1.00 | 14.15 | A | C |
| ATOM | 2105 | O | ILE | A | 277 | 21.596 | −44.354 | −11.478 | 1.00 | 14.38 | A | O |
| ATOM | 2106 | N | GLN | A | 278 | 20.762 | −42.921 | −9.973 | 1.00 | 14.64 | A | N |
| ATOM | 2107 | CA | GLN | A | 278 | 21.044 | −43.832 | −8.881 | 1.00 | 15.29 | A | C |
| ATOM | 2108 | CB | GLN | A | 278 | 20.665 | −43.220 | −7.546 | 1.00 | 15.95 | A | C |
| ATOM | 2109 | CG | GLN | A | 278 | 21.138 | −44.034 | −6.336 | 1.00 | 16.69 | A | C |
| ATOM | 2110 | CD | GLN | A | 278 | 22.656 | −44.231 | −6.267 | 1.00 | 16.71 | A | C |
| ATOM | 2111 | OE1 | GLN | A | 278 | 23.418 | −43.326 | −5.935 | 1.00 | 16.42 | A | O |
| ATOM | 2112 | NE2 | GLN | A | 278 | 23.084 | −45.437 | −6.552 | 1.00 | 17.15 | A | N |
| ATOM | 2113 | C | GLN | A | 278 | 20.343 | −45.163 | −9.067 | 1.00 | 15.22 | A | C |
| ATOM | 2114 | O | GLN | A | 278 | 20.930 | −46.204 | −8.832 | 1.00 | 15.46 | A | O |
| ATOM | 2115 | N | ALA | A | 279 | 19.100 | −45.141 | −9.522 | 1.00 | 15.50 | A | N |
| ATOM | 2116 | CA | ALA | A | 279 | 18.344 | −46.381 | −9.668 | 1.00 | 15.59 | A | C |
| ATOM | 2117 | CB | ALA | A | 279 | 16.872 | −46.074 | −9.868 | 1.00 | 15.79 | A | C |
| ATOM | 2118 | C | ALA | A | 279 | 18.870 | −47.272 | −10.789 | 1.00 | 15.64 | A | C |
| ATOM | 2119 | O | ALA | A | 279 | 18.837 | −48.496 | −10.692 | 1.00 | 16.17 | A | O |
| ATOM | 2120 | N | ALA | A | 280 | 19.357 | −46.670 | −11.858 | 1.00 | 15.94 | A | N |
| ATOM | 2121 | CA | ALA | A | 280 | 19.950 | −47.456 | −12.910 | 1.00 | 16.38 | A | C |
| ATOM | 2122 | CB | ALA | A | 280 | 20.116 | −46.649 | −14.174 | 1.00 | 16.73 | A | C |
| ATOM | 2123 | C | ALA | A | 280 | 21.286 | −47.972 | −12.446 | 1.00 | 16.78 | A | C |
| ATOM | 2124 | O | ALA | A | 280 | 21.616 | −49.113 | −12.734 | 1.00 | 17.18 | A | O |
| ATOM | 2125 | N | THR | A | 281 | 22.054 | −47.145 | −11.732 | 1.00 | 17.16 | A | N |
| ATOM | 2126 | CA | THR | A | 281 | 23.322 | −47.592 | −11.185 | 1.00 | 17.22 | A | C |
| ATOM | 2127 | CB | THR | A | 281 | 23.996 | −46.539 | −10.315 | 1.00 | 17.66 | A | C |
| ATOM | 2128 | OG1 | THR | A | 281 | 24.434 | −45.442 | −11.137 | 1.00 | 17.89 | A | O |
| ATOM | 2129 | CG2 | THR | A | 281 | 25.202 | −47.154 | −9.572 | 1.00 | 17.44 | A | C |
| ATOM | 2130 | C | THR | A | 281 | 23.074 | −48.819 | −10.355 | 1.00 | 17.79 | A | C |
| ATOM | 2131 | O | THR | A | 281 | 23.709 | −49.845 | −10.578 | 1.00 | 18.78 | A | O |
| ATOM | 2132 | N | ASP | A | 282 | 22.119 | −48.745 | −9.432 | 1.00 | 18.24 | A | N |
| ATOM | 2133 | CA | ASP | A | 282 | 21.722 | −49.930 | −8.616 | 1.00 | 18.73 | A | C |
| ATOM | 2134 | CB | ASP | A | 282 | 20.399 | −49.683 | −7.892 | 1.00 | 17.78 | A | C |
| ATOM | 2135 | CG | ASP | A | 282 | 20.513 | −48.689 | −6.767 | 1.00 | 17.00 | A | C |
| ATOM | 2136 | OD1 | ASP | A | 282 | 21.661 | −48.244 | −6.447 | 1.00 | 15.10 | A | O |
| ATOM | 2137 | OD2 | ASP | A | 282 | 19.413 | −48.384 | −6.218 | 1.00 | 16.44 | A | O |
| ATOM | 2138 | C | ASP | A | 282 | 21.538 | −51.236 | −9.375 | 1.00 | 19.72 | A | C |
| ATOM | 2139 | O | ASP | A | 282 | 21.845 | −52.281 | −8.873 | 1.00 | 19.64 | A | O |
| ATOM | 2140 | N | LEU | A | 283 | 20.992 | −51.163 | −10.575 | 1.00 | 22.83 | A | N |
| ATOM | 2141 | CA | LEU | A | 283 | 20.581 | −52.346 | −11.323 | 1.00 | 23.96 | A | C |
| ATOM | 2142 | CB | LEU | A | 283 | 19.265 | −52.043 | −12.036 | 1.00 | 24.88 | A | C |
| ATOM | 2143 | CG | LEU | A | 283 | 18.029 | −51.915 | −11.148 | 1.00 | 25.50 | A | C |
| ATOM | 2144 | CD1 | LEU | A | 283 | 16.958 | −51.126 | −11.898 | 1.00 | 26.24 | A | C |
| ATOM | 2145 | CD2 | LEU | A | 283 | 17.524 | −53.299 | −10.763 | 1.00 | 25.11 | A | C |
| ATOM | 2146 | C | LEU | A | 283 | 21.554 | −52.812 | −12.388 | 1.00 | 23.80 | A | C |
| ATOM | 2147 | O | LEU | A | 283 | 21.489 | −53.966 | −12.790 | 1.00 | 23.32 | A | O |
| ATOM | 2148 | N | TYR | A | 284 | 22.385 | −51.915 | −12.904 | 1.00 | 24.03 | A | N |
| ATOM | 2149 | CA | TYR | A | 284 | 23.184 | −52.237 | −14.085 | 1.00 | 25.72 | A | C |
| ATOM | 2150 | CB | TYR | A | 284 | 22.597 | −51.570 | −15.350 | 1.00 | 25.41 | A | C |
| ATOM | 2151 | CG | TYR | A | 284 | 21.211 | −52.073 | −15.711 | 1.00 | 25.02 | A | C |
| ATOM | 2152 | CD1 | TYR | A | 284 | 21.037 | −53.340 | −16.265 | 1.00 | 24.40 | A | C |
| ATOM | 2153 | CE1 | TYR | A | 284 | 19.766 | −53.811 | −16.579 | 1.00 | 25.54 | A | C |
| ATOM | 2154 | CZ | TYR | A | 284 | 18.630 | −53.007 | −16.344 | 1.00 | 24.69 | A | C |
| ATOM | 2155 | OH | TYR | A | 284 | 17.349 | −53.493 | −16.656 | 1.00 | 23.49 | A | O |
| ATOM | 2156 | CE2 | TYR | A | 284 | 18.801 | −51.748 | −15.782 | 1.00 | 23.41 | A | C |
| ATOM | 2157 | CD2 | TYR | A | 284 | 20.071 | −51.292 | −15.469 | 1.00 | 23.48 | A | C |
| ATOM | 2158 | C | TYR | A | 284 | 24.632 | −51.848 | −13.937 | 1.00 | 27.83 | A | C |
| ATOM | 2159 | O | TYR | A | 284 | 25.428 | −52.120 | −14.841 | 1.00 | 29.91 | A | O |
| ATOM | 2160 | N | GLY | A | 285 | 24.982 | −51.213 | −12.822 | 1.00 | 27.88 | A | N |
| ATOM | 2161 | CA | GLY | A | 285 | 26.361 | −50.831 | −12.567 | 1.00 | 29.33 | A | C |
| ATOM | 2162 | C | GLY | A | 285 | 26.614 | −49.435 | −13.076 | 1.00 | 32.33 | A | C |
| ATOM | 2163 | O | GLY | A | 285 | 26.058 | −49.040 | −14.098 | 1.00 | 36.64 | A | O |
| ATOM | 2164 | N | ALA | A | 286 | 27.481 | −48.698 | −12.392 | 1.00 | 32.78 | A | N |
| ATOM | 2165 | CA | ALA | A | 286 | 27.576 | −47.250 | −12.579 | 1.00 | 33.56 | A | C |
| ATOM | 2166 | CB | ALA | A | 286 | 28.379 | −46.627 | −11.440 | 1.00 | 34.16 | A | C |
| ATOM | 2167 | C | ALA | A | 286 | 28.143 | −46.799 | −13.923 | 1.00 | 33.26 | A | C |
| ATOM | 2168 | O | ALA | A | 286 | 28.010 | −45.618 | −14.292 | 1.00 | 34.56 | A | O |
| ATOM | 2169 | N | ASN | A | 287 | 28.772 | −47.707 | −14.656 | 1.00 | 31.09 | A | N |
| ATOM | 2170 | CA | ASN | A | 287 | 29.308 | −47.330 | −15.949 | 1.00 | 29.96 | A | C |
| ATOM | 2171 | CB | ASN | A | 287 | 30.821 | −47.577 | −15.965 | 1.00 | 30.24 | A | C |
| ATOM | 2172 | CG | ASN | A | 287 | 31.522 | −46.889 | −17.130 | 1.00 | 30.47 | A | C |
| ATOM | 2173 | OD1 | ASN | A | 287 | 31.112 | −45.823 | −17.601 | 1.00 | 26.91 | A | O |
| ATOM | 2174 | ND2 | ASN | A | 287 | 32.576 | −47.529 | −17.626 | 1.00 | 31.27 | A | N |
| ATOM | 2175 | C | ASN | A | 287 | 28.568 | −48.065 | −17.076 | 1.00 | 27.79 | A | C |
| ATOM | 2176 | O | ASN | A | 287 | 29.058 | −48.213 | −18.185 | 1.00 | 26.46 | A | O |
| ATOM | 2177 | N | SER | A | 288 | 27.350 | −48.482 | −16.802 | 1.00 | 27.08 | A | N |
| ATOM | 2178 | CA | SER | A | 288 | 26.584 | −49.237 | −17.781 | 1.00 | 26.73 | A | C |
| ATOM | 2179 | CB | SER | A | 288 | 25.406 | −49.971 | −17.108 | 1.00 | 27.43 | A | C |
| ATOM | 2180 | OG | SER | A | 288 | 24.598 | −49.085 | −16.318 | 1.00 | 28.18 | A | O |
| ATOM | 2181 | C | SER | A | 288 | 26.070 | −48.333 | −18.879 | 1.00 | 24.80 | A | C |
| ATOM | 2182 | O | SER | A | 288 | 26.043 | −47.106 | −18.746 | 1.00 | 23.67 | A | O |
| ATOM | 2183 | N | SER | A | 289 | 25.657 | −48.976 | −19.963 | 1.00 | 24.24 | A | N |
| ATOM | 2184 | CA | SER | A | 289 | 24.931 | −48.317 | −21.052 | 1.00 | 24.51 | A | C |
| ATOM | 2185 | CB | SER | A | 289 | 24.697 | −49.308 | −22.203 | 1.00 | 23.11 | A | C |
| ATOM | 2186 | OG | SER | A | 289 | 24.340 | −50.588 | −21.729 | 1.00 | 21.73 | A | O |
| ATOM | 2187 | C | SER | A | 289 | 23.576 | −47.698 | −20.591 | 1.00 | 25.23 | A | C |
| ATOM | 2188 | O | SER | A | 289 | 23.156 | −46.616 | −21.073 | 1.00 | 27.12 | A | O |
| ATOM | 2189 | N | GLN | A | 290 | 22.919 | −48.376 | −19.656 | 1.00 | 23.65 | A | N |
| ATOM | 2190 | CA | GLN | A | 290 | 21.640 | −47.929 | −19.149 | 1.00 | 23.31 | A | C |
| ATOM | 2191 | CB | GLN | A | 290 | 21.030 | −48.994 | −18.253 | 1.00 | 22.58 | A | C |
| ATOM | 2192 | CG | GLN | A | 290 | 20.519 | −50.209 | −19.023 | 1.00 | 22.68 | A | C |
| ATOM | 2193 | CD | GLN | A | 290 | 21.505 | −51.364 | −19.101 | 1.00 | 22.39 | A | C |
| ATOM | 2194 | OE1 | GLN | A | 290 | 22.711 | −51.186 | −19.035 | 1.00 | 23.40 | A | O |
| ATOM | 2195 | NE2 | GLN | A | 290 | 20.980 | −52.561 | −19.222 | 1.00 | 22.20 | A | N |
| ATOM | 2196 | C | GLN | A | 290 | 21.855 | −46.649 | −18.386 | 1.00 | 24.05 | A | C |
| ATOM | 2197 | O | GLN | A | 290 | 21.177 | −45.636 | −18.626 | 1.00 | 23.70 | A | O |
| ATOM | 2198 | N | VAL | A | 291 | 22.836 | −46.672 | −17.493 | 1.00 | 24.02 | A | N |
| ATOM | 2199 | CA | VAL | A | 291 | 23.154 | −45.475 | −16.754 | 1.00 | 23.78 | A | C |
| ATOM | 2200 | CB | VAL | A | 291 | 24.348 | −45.668 | −15.809 | 1.00 | 24.04 | A | C |
| ATOM | 2201 | CG1 | VAL | A | 291 | 24.995 | −44.332 | −15.456 | 1.00 | 23.39 | A | C |
| ATOM | 2202 | CG2 | VAL | A | 291 | 23.882 | −46.376 | −14.555 | 1.00 | 24.62 | A | C |
| ATOM | 2203 | C | VAL | A | 291 | 23.475 | −44.407 | −17.748 | 1.00 | 24.05 | A | C |
| ATOM | 2204 | O | VAL | A | 291 | 23.070 | −43.242 | −17.593 | 1.00 | 22.27 | A | O |
| ATOM | 2205 | N | ASN | A | 292 | 24.215 | −44.790 | −18.781 | 1.00 | 25.34 | A | N |
| ATOM | 2206 | CA | ASN | A | 292 | 24.673 | −43.762 | −19.676 | 1.00 | 26.69 | A | C |
| ATOM | 2207 | CB | ASN | A | 292 | 25.884 | −44.205 | −20.478 | 1.00 | 30.49 | A | C |
| ATOM | 2208 | CG | ASN | A | 292 | 26.820 | −43.042 | −20.731 | 1.00 | 35.02 | A | C |
| ATOM | 2209 | OD1 | ASN | A | 292 | 27.508 | −42.596 | −19.808 | 1.00 | 39.60 | A | O |
| ATOM | 2210 | ND2 | ASN | A | 292 | 26.793 | −42.487 | −21.952 | 1.00 | 35.12 | A | N |
| ATOM | 2211 | C | ASN | A | 292 | 23.553 | −43.174 | −20.551 | 1.00 | 23.23 | A | C |
| ATOM | 2212 | O | ASN | A | 292 | 23.547 | −41.978 | −20.829 | 1.00 | 21.51 | A | O |
| ATOM | 2213 | N | ALA | A | 293 | 22.594 | −44.013 | −20.932 | 1.00 | 20.89 | A | N |
| ATOM | 2214 | CA | ALA | A | 293 | 21.432 | −43.577 | −21.719 | 1.00 | 19.66 | A | C |
| ATOM | 2215 | CB | ALA | A | 293 | 20.559 | −44.780 | −22.043 | 1.00 | 20.04 | A | C |
| ATOM | 2216 | C | ALA | A | 293 | 20.614 | −42.573 | −20.957 | 1.00 | 18.76 | A | C |
| ATOM | 2217 | O | ALA | A | 293 | 20.105 | −41.606 | −21.499 | 1.00 | 17.05 | A | O |
| ATOM | 2218 | N | VAL | A | 294 | 20.461 | −42.866 | −19.669 | 1.00 | 18.81 | A | N |
| ATOM | 2219 | CA | VAL | A | 294 | 19.729 | −42.016 | −18.771 | 1.00 | 17.38 | A | C |
| ATOM | 2220 | CB | VAL | A | 294 | 19.646 | −42.671 | −17.382 | 1.00 | 16.37 | A | C |
| ATOM | 2221 | CG1 | VAL | A | 294 | 19.185 | −41.672 | −16.324 | 1.00 | 16.38 | A | C |
| ATOM | 2222 | CG2 | VAL | A | 294 | 18.736 | −43.875 | −17.422 | 1.00 | 15.72 | A | C |
| ATOM | 2223 | C | VAL | A | 294 | 20.396 | −40.645 | −18.748 | 1.00 | 17.76 | A | C |
| ATOM | 2224 | O | VAL | A | 294 | 19.713 | −39.640 | −18.840 | 1.00 | 17.49 | A | O |
| ATOM | 2225 | N | LYS | A | 295 | 21.727 | −40.613 | −18.654 | 1.00 | 18.95 | A | N |
| ATOM | 2226 | CA | LYS | A | 295 | 22.461 | −39.349 | −18.560 | 1.00 | 19.76 | A | C |
| ATOM | 2227 | CB | LYS | A | 295 | 23.964 | −39.577 | −18.381 | 1.00 | 21.38 | A | C |
| ATOM | 2228 | CG | LYS | A | 295 | 24.399 | −39.923 | −16.961 | 1.00 | 22.34 | A | C |
| ATOM | 2229 | CD | LYS | A | 295 | 25.900 | −40.268 | −16.887 | 1.00 | 23.25 | A | C |
| ATOM | 2230 | CE | LYS | A | 295 | 26.277 | −40.738 | −15.476 | 1.00 | 23.34 | A | C |
| ATOM | 2231 | NZ | LYS | A | 295 | 27.731 | −40.846 | −15.162 | 1.00 | 22.84 | A | N |
| ATOM | 2232 | C | LYS | A | 295 | 22.239 | −38.480 | −19.774 | 1.00 | 19.36 | A | C |
| ATOM | 2233 | O | LYS | A | 295 | 21.916 | −37.284 | −19.655 | 1.00 | 18.79 | A | O |
| ATOM | 2234 | N | LYS | A | 296 | 22.394 | −39.073 | −20.944 | 1.00 | 19.79 | A | N |
| ATOM | 2235 | CA | LYS | A | 296 | 22.200 | −38.316 | −22.176 | 1.00 | 21.18 | A | C |
| ATOM | 2236 | CB | LYS | A | 296 | 22.618 | −39.143 | −23.379 | 1.00 | 23.67 | A | C |
| ATOM | 2237 | CG | LYS | A | 296 | 24.126 | −39.307 | −23.392 | 1.00 | 27.94 | A | C |
| ATOM | 2238 | CD | LYS | A | 296 | 24.657 | −39.899 | −24.691 | 1.00 | 32.89 | A | C |
| ATOM | 2239 | CE | LYS | A | 296 | 26.166 | −39.650 | −24.836 | 1.00 | 37.16 | A | C |
| ATOM | 2240 | NZ | LYS | A | 296 | 26.520 | −38.247 | −25.268 | 1.00 | 38.34 | A | N |
| ATOM | 2241 | C | LYS | A | 296 | 20.785 | −37.754 | −22.325 | 1.00 | 19.72 | A | C |
| ATOM | 2242 | O | LYS | A | 296 | 20.603 | −36.553 | −22.615 | 1.00 | 18.85 | A | O |
| ATOM | 2243 | N | ALA | A | 297 | 19.804 | −38.618 | −22.087 | 1.00 | 18.65 | A | N |
| ATOM | 2244 | CA | ALA | A | 297 | 18.395 | −38.236 | −22.059 | 1.00 | 18.30 | A | C |
| ATOM | 2245 | CB | ALA | A | 297 | 17.544 | −39.389 | −21.570 | 1.00 | 19.00 | A | C |
| ATOM | 2246 | C | ALA | A | 297 | 18.141 | −37.014 | −21.207 | 1.00 | 18.02 | A | C |
| ATOM | 2247 | O | ALA | A | 297 | 17.575 | −36.041 | −21.683 | 1.00 | 18.61 | A | O |
| ATOM | 2248 | N | TYR | A | 298 | 18.578 | −37.016 | −19.960 | 1.00 | 17.55 | A | N |
| ATOM | 2249 | CA | TYR | A | 298 | 18.361 | −35.808 | −19.165 | 1.00 | 17.98 | A | C |
| ATOM | 2250 | CB | TYR | A | 298 | 18.595 | −36.043 | −17.662 | 1.00 | 17.98 | A | C |
| ATOM | 2251 | CG | TYR | A | 298 | 17.361 | −36.698 | −17.049 | 1.00 | 17.27 | A | C |
| ATOM | 2252 | CD1 | TYR | A | 298 | 16.272 | −35.926 | −16.677 | 1.00 | 16.76 | A | C |
| ATOM | 2253 | CE1 | TYR | A | 298 | 15.140 | −36.498 | −16.150 | 1.00 | 16.52 | A | C |
| ATOM | 2254 | CZ | TYR | A | 298 | 15.057 | −37.866 | −16.011 | 1.00 | 16.59 | A | C |
| ATOM | 2255 | OH | TYR | A | 298 | 13.898 | −38.414 | −15.489 | 1.00 | 15.41 | A | O |
| ATOM | 2256 | CE2 | TYR | A | 298 | 16.135 | −38.665 | −16.381 | 1.00 | 16.79 | A | C |
| ATOM | 2257 | CD2 | TYR | A | 298 | 17.268 | −38.079 | −16.910 | 1.00 | 16.48 | A | C |
| ATOM | 2258 | C | TYR | A | 298 | 19.123 | −34.601 | −19.686 | 1.00 | 17.99 | A | C |
| ATOM | 2259 | O | TYR | A | 298 | 18.623 | −33.470 | −19.572 | 1.00 | 19.07 | A | O |
| ATOM | 2260 | N | THR | A | 299 | 20.307 | −34.828 | −20.255 | 1.00 | 17.65 | A | N |
| ATOM | 2261 | CA | THR | A | 299 | 21.056 | −33.746 | −20.902 | 1.00 | 17.98 | A | C |
| ATOM | 2262 | CB | THR | A | 299 | 22.472 | −34.217 | −21.368 | 1.00 | 17.57 | A | C |
| ATOM | 2263 | OG1 | THR | A | 299 | 23.235 | −34.706 | −20.250 | 1.00 | 16.90 | A | O |
| ATOM | 2264 | CG2 | THR | A | 299 | 23.217 | −33.076 | −22.047 | 1.00 | 16.53 | A | C |
| ATOM | 2265 | C | THR | A | 299 | 20.231 | −33.206 | −22.118 | 1.00 | 18.22 | A | C |
| ATOM | 2266 | O | THR | A | 299 | 20.019 | −31.985 | −22.289 | 1.00 | 16.64 | A | O |
| ATOM | 2267 | N | ALA | A | 300 | 19.744 | −34.132 | −22.938 | 1.00 | 18.02 | A | N |
| ATOM | 2268 | CA | ALA | A | 300 | 18.960 | −33.751 | −24.087 | 1.00 | 18.32 | A | C |
| ATOM | 2269 | CB | ALA | A | 300 | 18.408 | −34.982 | −24.786 | 1.00 | 18.60 | A | C |
| ATOM | 2270 | C | ALA | A | 300 | 17.840 | −32.798 | −23.701 | 1.00 | 18.60 | A | C |
| ATOM | 2271 | O | ALA | A | 300 | 17.659 | −31.798 | −24.366 | 1.00 | 20.19 | A | O |
| ATOM | 2272 | N | VAL | A | 301 | 17.101 | −33.074 | −22.630 | 1.00 | 18.61 | A | N |
| ATOM | 2273 | CA | VAL | A | 301 | 15.996 | −32.189 | −22.240 | 1.00 | 18.71 | A | C |
| ATOM | 2274 | CB | VAL | A | 301 | 14.856 | −32.940 | −21.538 | 1.00 | 19.41 | A | C |
| ATOM | 2275 | CG1 | VAL | A | 301 | 14.404 | −34.146 | −22.338 | 1.00 | 19.54 | A | C |
| ATOM | 2276 | CG2 | VAL | A | 301 | 15.298 | −33.395 | −20.170 | 1.00 | 20.37 | A | C |
| ATOM | 2277 | C | VAL | A | 301 | 16.457 | −31.043 | −21.350 | 1.00 | 18.65 | A | C |
| ATOM | 2278 | O | VAL | A | 301 | 15.646 | −30.314 | −20.777 | 1.00 | 18.51 | A | O |
| ATOM | 2279 | N | GLY | A | 302 | 17.758 | −30.871 | −21.231 | 1.00 | 19.01 | A | N |
| ATOM | 2280 | CA | GLY | A | 302 | 18.282 | −29.621 | −20.718 | 1.00 | 19.86 | A | C |
| ATOM | 2281 | C | GLY | A | 302 | 18.370 | −29.640 | −19.219 | 1.00 | 21.35 | A | C |
| ATOM | 2282 | O | GLY | A | 302 | 18.411 | −28.580 | −18.581 | 1.00 | 20.19 | A | O |
| ATOM | 2283 | N | VAL | A | 303 | 18.392 | −30.858 | −18.659 | 1.00 | 23.47 | A | N |
| ATOM | 2284 | CA | VAL | A | 303 | 18.503 | −31.054 | −17.208 | 1.00 | 23.96 | A | C |
| ATOM | 2285 | CB | VAL | A | 303 | 17.367 | −31.958 | −16.677 | 1.00 | 23.91 | A | C |
| ATOM | 2286 | CG1 | VAL | A | 303 | 17.488 | −32.161 | −15.169 | 1.00 | 24.18 | A | C |
| ATOM | 2287 | CG2 | VAL | A | 303 | 16.027 | −31.330 | −16.985 | 1.00 | 24.04 | A | C |
| ATOM | 2288 | C | VAL | A | 303 | 19.868 | −31.647 | −16.845 | 1.00 | 23.68 | A | C |
| ATOM | 2289 | O | VAL | A | 303 | 20.199 | −32.752 | −17.266 | 1.00 | 22.79 | A | O |
| ATOM | 2290 | N | ASN | A | 304 | 20.630 | −30.924 | −16.033 | 1.00 | 24.44 | A | N |
| ATOM | 2291 | CA | ASN | A | 304 | 21.971 | −31.369 | −15.668 | 1.00 | 27.13 | A | C |
| ATOM | 2292 | CB | ASN | A | 304 | 22.976 | −30.333 | −16.137 | 1.00 | 27.63 | A | C |
| ATOM | 2293 | CG | ASN | A | 304 | 22.782 | −29.969 | −17.602 | 1.00 | 28.08 | A | C |
| ATOM | 2294 | OD1 | ASN | A | 304 | 22.758 | −30.844 | −18.497 | 1.00 | 26.81 | A | O |
| ATOM | 2295 | ND2 | ASN | A | 304 | 22.632 | −28.666 | −17.855 | 1.00 | 27.74 | A | N |
| ATOM | 2296 | C | ASN | A | 304 | 22.203 | −31.666 | −14.188 | 1.00 | 27.90 | A | C |
| ATOM | 2297 | O | ASN | A | 304 | 21.646 | −31.075 | −13.248 | 1.00 | 28.54 | A | O |
| ATOM | 2298 | OXT | ASN | A | 304 | 23.015 | −32.544 | −13.941 | 1.00 | 27.35 | A | O |
| ATOM | 2299 | N | ALA | B | 1 | −27.118 | −11.588 | 16.141 | 1.00 | 36.52 | B | N |
| ATOM | 2300 | CA | ALA | B | 1 | −26.133 | −11.218 | 17.198 | 1.00 | 37.78 | B | C |
| ATOM | 2301 | CB | ALA | B | 1 | −25.839 | −12.399 | 18.109 | 1.00 | 36.98 | B | C |
| ATOM | 2302 | C | ALA | B | 1 | −24.878 | −10.774 | 16.492 | 1.00 | 39.27 | B | C |
| ATOM | 2303 | O | ALA | B | 1 | −24.736 | −11.016 | 15.302 | 1.00 | 43.37 | B | O |
| ATOM | 2304 | N | THR | B | 2 | −23.958 | −10.156 | 17.220 | 1.00 | 39.24 | B | N |
| ATOM | 2305 | CA | THR | B | 2 | −22.776 | −9.556 | 16.609 | 1.00 | 38.21 | B | C |
| ATOM | 2306 | CB | THR | B | 2 | −22.792 | −8.015 | 16.742 | 1.00 | 38.56 | B | C |
| ATOM | 2307 | OG1 | THR | B | 2 | −23.817 | −7.475 | 15.901 | 1.00 | 37.57 | B | O |
| ATOM | 2308 | CG2 | THR | B | 2 | −21.445 | −7.408 | 16.322 | 1.00 | 39.78 | B | C |
| ATOM | 2309 | C | THR | B | 2 | −21.477 | −10.140 | 17.183 | 1.00 | 37.53 | B | C |
| ATOM | 2310 | O | THR | B | 2 | −21.027 | −9.766 | 18.266 | 1.00 | 32.77 | B | O |
| ATOM | 2311 | N | GLY | B | 3 | −20.864 | −11.037 | 16.413 | 1.00 | 38.90 | B | N |
| ATOM | 2312 | CA | GLY | B | 3 | −19.625 | −11.693 | 16.822 | 1.00 | 39.37 | B | C |
| ATOM | 2313 | C | GLY | B | 3 | −18.385 | −10.922 | 16.439 | 1.00 | 37.66 | B | C |
| ATOM | 2314 | O | GLY | B | 3 | −18.379 | −10.180 | 15.475 | 1.00 | 38.38 | B | O |
| ATOM | 2315 | N | THR | B | 4 | −17.325 | −11.116 | 17.193 | 1.00 | 37.26 | B | N |
| ATOM | 2316 | CA | THR | B | 4 | −16.066 | −10.493 | 16.873 | 1.00 | 39.03 | B | C |
| ATOM | 2317 | CB | THR | B | 4 | −15.631 | −9.497 | 17.978 | 1.00 | 38.57 | B | C |
| ATOM | 2318 | OG1 | THR | B | 4 | −14.242 | −9.154 | 17.811 | 1.00 | 38.10 | B | O |
| ATOM | 2319 | CG2 | THR | B | 4 | −15.829 | −10.089 | 19.342 | 1.00 | 36.87 | B | C |
| ATOM | 2320 | C | THR | B | 4 | −14.993 | −11.568 | 16.642 | 1.00 | 39.28 | B | C |
| ATOM | 2321 | O | THR | B | 4 | −15.018 | −12.622 | 17.277 | 1.00 | 37.20 | B | O |
| ATOM | 2322 | N | GLY | B | 5 | −14.064 | −11.284 | 15.725 | 1.00 | 38.89 | B | N |
| ATOM | 2323 | CA | GLY | B | 5 | −12.864 | −12.101 | 15.541 | 1.00 | 37.29 | B | C |
| ATOM | 2324 | C | GLY | B | 5 | −11.851 | −11.559 | 14.531 | 1.00 | 35.59 | B | C |
| ATOM | 2325 | O | GLY | B | 5 | −12.190 | −10.789 | 13.611 | 1.00 | 34.39 | B | O |
| ATOM | 2326 | N | LYS | B | 6 | −10.601 | −11.982 | 14.704 | 1.00 | 32.33 | B | N |
| ATOM | 2327 | CA | LYS | B | 6 | −9.501 | −11.572 | 13.834 | 1.00 | 30.16 | B | C |
| ATOM | 2328 | CB | LYS | B | 6 | −8.164 | −11.793 | 14.523 | 1.00 | 28.67 | B | C |
| ATOM | 2329 | CG | LYS | B | 6 | −8.056 | −11.021 | 15.798 | 1.00 | 29.42 | B | C |
| ATOM | 2330 | CD | LYS | B | 6 | −6.667 | −11.050 | 16.395 | 1.00 | 31.17 | B | C |
| ATOM | 2331 | CE | LYS | B | 6 | −6.396 | −9.726 | 17.100 | 1.00 | 31.96 | B | C |
| ATOM | 2332 | NZ | LYS | B | 6 | −5.427 | −9.958 | 18.189 | 1.00 | 33.82 | B | N |
| ATOM | 2333 | C | LYS | B | 6 | −9.528 | −12.360 | 12.547 | 1.00 | 28.68 | B | C |
| ATOM | 2334 | O | LYS | B | 6 | −9.889 | −13.529 | 12.555 | 1.00 | 29.96 | B | O |
| ATOM | 2335 | N | GLY | B | 7 | −9.141 | −11.718 | 11.447 | 1.00 | 26.88 | B | N |
| ATOM | 2336 | CA | GLY | B | 7 | −9.082 | −12.374 | 10.150 | 1.00 | 24.95 | B | C |
| ATOM | 2337 | C | GLY | B | 7 | −7.727 | −13.011 | 10.017 | 1.00 | 23.60 | B | C |
| ATOM | 2338 | O | GLY | B | 7 | −6.884 | −12.864 | 10.883 | 1.00 | 23.09 | B | O |
| ATOM | 2339 | N | VAL | B | 8 | −7.515 | −13.700 | 8.912 | 1.00 | 22.56 | B | N |
| ATOM | 2340 | CA | VAL | B | 8 | −6.255 | −14.375 | 8.641 | 1.00 | 22.38 | B | C |
| ATOM | 2341 | CB | VAL | B | 8 | −6.215 | −14.891 | 7.197 | 1.00 | 22.59 | B | C |
| ATOM | 2342 | CG1 | VAL | B | 8 | −4.869 | −15.545 | 6.892 | 1.00 | 22.83 | B | C |
| ATOM | 2343 | CG2 | VAL | B | 8 | −7.357 | −15.870 | 6.940 | 1.00 | 22.50 | B | C |
| ATOM | 2344 | C | VAL | B | 8 | −5.041 | −13.479 | 8.847 | 1.00 | 22.68 | B | C |
| ATOM | 2345 | O | VAL | B | 8 | −4.003 | −13.935 | 9.296 | 1.00 | 21.46 | B | O |
| ATOM | 2346 | N | LEU | B | 9 | −5.173 | −12.201 | 8.498 | 1.00 | 24.54 | B | N |
| ATOM | 2347 | CA | LEU | B | 9 | −4.063 | −11.242 | 8.600 | 1.00 | 24.12 | B | C |
| ATOM | 2348 | CB | LEU | B | 9 | −4.135 | −10.261 | 7.432 | 1.00 | 23.12 | B | C |
| ATOM | 2349 | CG | LEU | B | 9 | −3.359 | −10.564 | 6.154 | 1.00 | 21.95 | B | C |
| ATOM | 2350 | CD1 | LEU | B | 9 | −3.041 | −12.036 | 6.027 | 1.00 | 20.77 | B | C |
| ATOM | 2351 | CD2 | LEU | B | 9 | −4.141 | −9.999 | 4.961 | 1.00 | 21.42 | B | C |
| ATOM | 2352 | C | LEU | B | 9 | −4.046 | −10.456 | 9.890 | 1.00 | 25.26 | B | C |
| ATOM | 2353 | O | LEU | B | 9 | −3.442 | −9.408 | 9.933 | 1.00 | 28.39 | B | O |
| ATOM | 2354 | N | GLY | B | 10 | −4.723 | −10.932 | 10.930 | 1.00 | 26.73 | B | N |
| ATOM | 2355 | CA | GLY | B | 10 | −4.586 | −10.358 | 12.279 | 1.00 | 26.45 | B | C |
| ATOM | 2356 | C | GLY | B | 10 | −5.481 | −9.174 | 12.586 | 1.00 | 26.20 | B | C |
| ATOM | 2357 | O | GLY | B | 10 | −5.297 | −8.504 | 13.614 | 1.00 | 24.13 | B | O |
| ATOM | 2358 | N | ASP | B | 11 | −6.481 | −8.966 | 11.727 | 1.00 | 26.26 | B | N |
| ATOM | 2359 | CA | ASP | B | 11 | −7.342 | −7.781 | 11.761 | 1.00 | 26.23 | B | C |
| ATOM | 2360 | CB | ASP | B | 11 | −7.410 | −7.155 | 10.356 | 1.00 | 24.59 | B | C |
| ATOM | 2361 | CG | ASP | B | 11 | −7.968 | −8.109 | 9.295 | 1.00 | 24.79 | B | C |
| ATOM | 2362 | OD1 | ASP | B | 11 | −7.465 | −9.238 | 9.139 | 1.00 | 25.46 | B | O |
| ATOM | 2363 | OD2 | ASP | B | 11 | −8.922 | −7.739 | 8.596 | 1.00 | 23.52 | B | O |
| ATOM | 2364 | C | ASP | B | 11 | −8.767 | −8.052 | 12.313 | 1.00 | 28.01 | B | C |
| ATOM | 2365 | O | ASP | B | 11 | −9.541 | −8.838 | 11.737 | 1.00 | 29.86 | B | O |
| ATOM | 2366 | N | THR | B | 12 | −9.127 | −7.357 | 13.397 | 1.00 | 28.40 | B | N |
| ATOM | 2367 | CA | THR | B | 12 | −10.417 | −7.555 | 14.073 | 1.00 | 27.68 | B | C |
| ATOM | 2368 | CB | THR | B | 12 | −10.402 | −6.914 | 15.465 | 1.00 | 25.99 | B | C |
| ATOM | 2369 | OG1 | THR | B | 12 | −9.232 | −7.350 | 16.173 | 1.00 | 25.66 | B | O |
| ATOM | 2370 | CG2 | THR | B | 12 | −11.645 | −7.298 | 16.258 | 1.00 | 24.54 | B | C |
| ATOM | 2371 | C | THR | B | 12 | −11.633 | −7.041 | 13.285 | 1.00 | 29.85 | B | C |
| ATOM | 2372 | O | THR | B | 12 | −11.677 | −5.875 | 12.875 | 1.00 | 32.91 | B | O |
| ATOM | 2373 | N | LYS | B | 13 | −12.618 | −7.922 | 13.090 | 1.00 | 30.70 | B | N |
| ATOM | 2374 | CA | LYS | B | 13 | −13.867 | −7.580 | 12.402 | 1.00 | 31.16 | B | C |
| ATOM | 2375 | CB | LYS | B | 13 | −14.036 | −8.339 | 11.087 | 1.00 | 31.49 | B | C |
| ATOM | 2376 | CG | LYS | B | 13 | −12.756 | −8.854 | 10.458 | 1.00 | 32.70 | B | C |
| ATOM | 2377 | CD | LYS | B | 13 | −13.037 | −9.300 | 9.041 | 1.00 | 32.47 | B | C |
| ATOM | 2378 | CE | LYS | B | 13 | −11.763 | −9.658 | 8.315 | 1.00 | 32.16 | B | C |
| ATOM | 2379 | NZ | LYS | B | 13 | −10.786 | −8.555 | 8.175 | 1.00 | 31.87 | B | N |
| ATOM | 2380 | C | LYS | B | 13 | −15.052 | −7.967 | 13.238 | 1.00 | 33.23 | B | C |
| ATOM | 2381 | O | LYS | B | 13 | −14.940 | −8.784 | 14.157 | 1.00 | 39.07 | B | O |
| ATOM | 2382 | N | SER | B | 14 | −16.196 | −7.403 | 12.866 | 1.00 | 31.47 | B | N |
| ATOM | 2383 | CA | SER | B | 14 | −17.474 | −7.714 | 13.446 | 1.00 | 29.93 | B | C |
| ATOM | 2384 | CB | SER | B | 14 | −18.225 | −6.440 | 13.792 | 1.00 | 31.68 | B | C |
| ATOM | 2385 | OG | SER | B | 14 | −17.421 | −5.631 | 14.618 | 1.00 | 34.18 | B | O |
| ATOM | 2386 | C | SER | B | 14 | −18.244 | −8.385 | 12.377 | 1.00 | 28.96 | B | C |
| ATOM | 2387 | O | SER | B | 14 | −17.989 | −8.156 | 11.196 | 1.00 | 27.52 | B | O |
| ATOM | 2388 | N | PHE | B | 15 | −19.225 | −9.178 | 12.773 | 1.00 | 28.58 | B | N |
| ATOM | 2389 | CA | PHE | B | 15 | −20.097 | −9.822 | 11.797 | 1.00 | 28.92 | B | C |
| ATOM | 2390 | CB | PHE | B | 15 | −19.330 | −10.903 | 11.010 | 1.00 | 27.95 | B | C |
| ATOM | 2391 | CG | PHE | B | 15 | −18.345 | −11.675 | 11.853 | 1.00 | 26.41 | B | C |
| ATOM | 2392 | CD1 | PHE | B | 15 | −18.751 | −12.766 | 12.586 | 1.00 | 24.71 | B | C |
| ATOM | 2393 | CE1 | PHE | B | 15 | −17.861 | −13.459 | 13.368 | 1.00 | 24.48 | B | C |
| ATOM | 2394 | CZ | PHE | B | 15 | −16.548 | −13.067 | 13.437 | 1.00 | 25.51 | B | C |
| ATOM | 2395 | CE2 | PHE | B | 15 | −16.130 | −11.967 | 12.722 | 1.00 | 26.27 | B | C |
| ATOM | 2396 | CD2 | PHE | B | 15 | −17.028 | −11.281 | 11.934 | 1.00 | 26.09 | B | C |
| ATOM | 2397 | C | PHE | B | 15 | −21.283 | −10.421 | 12.515 | 1.00 | 30.15 | B | C |
| ATOM | 2398 | O | PHE | B | 15 | −21.231 | −10.661 | 13.735 | 1.00 | 28.64 | B | O |
| ATOM | 2399 | N | THR | B | 16 | −22.350 | −10.643 | 11.756 | 1.00 | 32.13 | B | N |
| ATOM | 2400 | CA | THR | B | 16 | −23.564 | −11.231 | 12.297 | 1.00 | 35.95 | B | C |
| ATOM | 2401 | CB | THR | B | 16 | −24.775 | −10.980 | 11.371 | 1.00 | 35.50 | B | C |
| ATOM | 2402 | OG1 | THR | B | 16 | −25.103 | −9.584 | 11.373 | 1.00 | 34.31 | B | O |
| ATOM | 2403 | CG2 | THR | B | 16 | −26.013 | −11.807 | 11.799 | 1.00 | 35.19 | B | C |
| ATOM | 2404 | C | THR | B | 16 | −23.380 | −12.745 | 12.499 | 1.00 | 40.78 | B | C |
| ATOM | 2405 | O | THR | B | 16 | −23.020 | −13.467 | 11.556 | 1.00 | 41.40 | B | O |
| ATOM | 2406 | N | THR | B | 17 | −23.627 | −13.198 | 13.732 | 1.00 | 42.02 | B | N |
| ATOM | 2407 | CA | THR | B | 17 | −23.766 | −14.616 | 14.057 | 1.00 | 43.39 | B | C |
| ATOM | 2408 | CB | THR | B | 17 | −22.819 | −14.986 | 15.206 | 1.00 | 42.77 | B | C |
| ATOM | 2409 | OG1 | THR | B | 17 | −23.194 | −14.299 | 16.410 | 1.00 | 40.33 | B | O |
| ATOM | 2410 | CG2 | THR | B | 17 | −21.392 | −14.597 | 14.826 | 1.00 | 42.32 | B | C |
| ATOM | 2411 | C | THR | B | 17 | −25.237 | −14.905 | 14.412 | 1.00 | 47.94 | B | C |
| ATOM | 2412 | O | THR | B | 17 | −26.098 | −14.036 | 14.216 | 1.00 | 53.17 | B | O |
| ATOM | 2413 | N | THR | B | 18 | −25.536 | −16.119 | 14.880 | 1.00 | 48.55 | B | N |
| ATOM | 2414 | CA | THR | B | 18 | −26.883 | −16.470 | 15.372 | 1.00 | 47.03 | B | C |
| ATOM | 2415 | CB | THR | B | 18 | −27.715 | −17.260 | 14.322 | 1.00 | 46.64 | B | C |
| ATOM | 2416 | OG1 | THR | B | 18 | −27.903 | −16.465 | 13.143 | 1.00 | 47.35 | B | O |
| ATOM | 2417 | CG2 | THR | B | 18 | −29.098 | −17.635 | 14.867 | 1.00 | 47.20 | B | C |
| ATOM | 2418 | C | THR | B | 18 | −26.781 | −17.247 | 16.695 | 1.00 | 49.03 | B | C |
| ATOM | 2419 | O | THR | B | 18 | −25.845 | −18.027 | 16.910 | 1.00 | 45.35 | B | O |
| ATOM | 2420 | N | GLN | B | 19 | −27.735 | −16.996 | 17.592 | 1.00 | 54.36 | B | N |
| ATOM | 2421 | CA | GLN | B | 19 | −27.790 | −17.676 | 18.879 | 1.00 | 54.96 | B | C |
| ATOM | 2422 | CB | GLN | B | 19 | −28.436 | −16.770 | 19.934 | 1.00 | 58.37 | B | C |
| ATOM | 2423 | CG | GLN | B | 19 | −27.996 | −17.086 | 21.361 | 1.00 | 62.57 | B | C |
| ATOM | 2424 | CD | GLN | B | 19 | −28.459 | −16.067 | 22.402 | 1.00 | 63.05 | B | C |
| ATOM | 2425 | OE1 | GLN | B | 19 | −27.896 | −14.971 | 22.517 | 1.00 | 60.83 | B | O |
| ATOM | 2426 | NE2 | GLN | B | 19 | −29.472 | −16.437 | 23.185 | 1.00 | 62.16 | B | N |
| ATOM | 2427 | C | GLN | B | 19 | −28.597 | −18.953 | 18.685 | 1.00 | 53.79 | B | C |
| ATOM | 2428 | O | GLN | B | 19 | −29.711 | −18.899 | 18.167 | 1.00 | 53.84 | B | O |
| ATOM | 2429 | N | SER | B | 20 | −28.016 | −20.097 | 19.046 | 1.00 | 54.80 | B | N |
| ATOM | 2430 | CA | SER | B | 20 | −28.744 | −21.381 | 19.062 | 1.00 | 54.29 | B | C |
| ATOM | 2431 | CB | SER | B | 20 | −28.141 | −22.387 | 18.081 | 1.00 | 53.79 | B | C |
| ATOM | 2432 | OG | SER | B | 20 | −29.021 | −23.476 | 17.888 | 1.00 | 49.78 | B | O |
| ATOM | 2433 | C | SER | B | 20 | −28.687 | −21.918 | 20.481 | 1.00 | 53.51 | B | C |
| ATOM | 2434 | O | SER | B | 20 | −27.696 | −22.552 | 20.883 | 1.00 | 51.94 | B | O |
| ATOM | 2435 | N | GLY | B | 21 | −29.746 | −21.617 | 21.235 | 1.00 | 52.98 | B | N |
| ATOM | 2436 | CA | GLY | B | 21 | −29.787 | −21.842 | 22.675 | 1.00 | 53.72 | B | C |
| ATOM | 2437 | C | GLY | B | 21 | −28.455 | −21.571 | 23.358 | 1.00 | 53.77 | B | C |
| ATOM | 2438 | O | GLY | B | 21 | −28.115 | −20.417 | 23.666 | 1.00 | 50.85 | B | O |
| ATOM | 2439 | N | SER | B | 22 | −27.690 | −22.641 | 23.549 | 1.00 | 53.12 | B | N |
| ATOM | 2440 | CA | SER | B | 22 | −26.521 | −22.621 | 24.419 | 1.00 | 59.41 | B | C |
| ATOM | 2441 | CB | SER | B | 22 | −26.174 | −24.057 | 24.820 | 1.00 | 59.03 | B | C |
| ATOM | 2442 | OG | SER | B | 22 | −25.106 | −24.053 | 25.747 | 1.00 | 61.97 | B | O |
| ATOM | 2443 | C | SER | B | 22 | −25.272 | −21.930 | 23.824 | 1.00 | 61.98 | B | C |
| ATOM | 2444 | O | SER | B | 22 | −24.452 | −21.348 | 24.563 | 1.00 | 61.14 | B | O |
| ATOM | 2445 | N | THR | B | 23 | −25.134 | −21.998 | 22.501 | 1.00 | 57.12 | B | N |
| ATOM | 2446 | CA | THR | B | 23 | −23.930 | −21.532 | 21.828 | 1.00 | 55.19 | B | C |
| ATOM | 2447 | CB | THR | B | 23 | −23.101 | −22.755 | 21.366 | 1.00 | 52.30 | B | C |
| ATOM | 2448 | OG1 | THR | B | 23 | −21.696 | −22.470 | 21.419 | 1.00 | 49.29 | B | O |
| ATOM | 2449 | CG2 | THR | B | 23 | −23.522 | −23.221 | 19.960 | 1.00 | 51.82 | B | C |
| ATOM | 2450 | C | THR | B | 23 | −24.344 | −20.627 | 20.655 | 1.00 | 55.21 | B | C |
| ATOM | 2451 | O | THR | B | 23 | −25.542 | −20.492 | 20.358 | 1.00 | 54.01 | B | O |
| ATOM | 2452 | N | TYR | B | 24 | −23.365 | −19.996 | 20.010 | 1.00 | 52.08 | B | N |
| ATOM | 2453 | CA | TYR | B | 24 | −23.622 | −19.217 | 18.798 | 1.00 | 50.27 | B | C |
| ATOM | 2454 | CB | TYR | B | 24 | −23.019 | −17.812 | 18.884 | 1.00 | 52.10 | B | C |
| ATOM | 2455 | CG | TYR | B | 24 | −23.470 | −16.931 | 20.029 | 1.00 | 51.06 | B | C |
| ATOM | 2456 | CD1 | TYR | B | 24 | −24.563 | −16.095 | 19.888 | 1.00 | 49.59 | B | C |
| ATOM | 2457 | CE1 | TYR | B | 24 | −24.967 | −15.278 | 20.923 | 1.00 | 52.70 | B | C |
| ATOM | 2458 | CZ | TYR | B | 24 | −24.268 | −15.276 | 22.121 | 1.00 | 52.07 | B | C |
| ATOM | 2459 | OH | TYR | B | 24 | −24.689 | −14.454 | 23.153 | 1.00 | 52.35 | B | O |
| ATOM | 2460 | CE2 | TYR | B | 24 | −23.160 | −16.085 | 22.277 | 1.00 | 50.76 | B | C |
| ATOM | 2461 | CD2 | TYR | B | 24 | −22.764 | −16.903 | 21.235 | 1.00 | 50.70 | B | C |
| ATOM | 2462 | C | TYR | B | 24 | −22.990 | −19.902 | 17.600 | 1.00 | 50.42 | B | C |
| ATOM | 2463 | O | TYR | B | 24 | −21.978 | −20.595 | 17.725 | 1.00 | 50.73 | B | O |
| ATOM | 2464 | N | GLN | B | 25 | −23.547 | −19.640 | 16.425 | 1.00 | 49.79 | B | N |
| ATOM | 2465 | CA | GLN | B | 25 | −23.089 | −20.271 | 15.193 | 1.00 | 51.16 | B | C |
| ATOM | 2466 | CB | GLN | B | 25 | −24.083 | −21.380 | 14.804 | 1.00 | 52.14 | B | C |
| ATOM | 2467 | CG | GLN | B | 25 | −25.550 | −20.946 | 14.773 | 1.00 | 51.47 | B | C |
| ATOM | 2468 | CD | GLN | B | 25 | −26.509 | −22.069 | 14.400 | 1.00 | 49.02 | B | C |
| ATOM | 2469 | OE1 | GLN | B | 25 | −26.386 | −23.186 | 14.879 | 1.00 | 43.29 | B | O |
| ATOM | 2470 | NE2 | GLN | B | 25 | −27.483 | −21.759 | 13.549 | 1.00 | 49.61 | B | N |
| ATOM | 2471 | C | GLN | B | 25 | −22.919 | −19.219 | 14.064 | 1.00 | 52.10 | B | C |
| ATOM | 2472 | O | GLN | B | 25 | −23.666 | −18.240 | 14.038 | 1.00 | 54.65 | B | O |
| ATOM | 2473 | N | LEU | B | 26 | −21.940 | −19.406 | 13.158 | 1.00 | 49.06 | B | N |
| ATOM | 2474 | CA | LEU | B | 26 | −21.703 | −18.464 | 12.035 | 1.00 | 45.19 | B | C |
| ATOM | 2475 | CB | LEU | B | 26 | −20.376 | −18.751 | 11.315 | 1.00 | 46.15 | B | C |
| ATOM | 2476 | CG | LEU | B | 26 | −19.037 | −18.374 | 11.951 | 1.00 | 45.52 | B | C |
| ATOM | 2477 | CD1 | LEU | B | 26 | −17.937 | −18.472 | 10.912 | 1.00 | 46.14 | B | C |
| ATOM | 2478 | CD2 | LEU | B | 26 | −19.057 | −16.972 | 12.511 | 1.00 | 44.30 | B | C |
| ATOM | 2479 | C | LEU | B | 26 | −22.827 | −18.489 | 11.006 | 1.00 | 44.22 | B | C |
| ATOM | 2480 | O | LEU | B | 26 | −22.671 | −19.012 | 9.903 | 1.00 | 45.46 | B | O |
| ATOM | 2481 | N | LYS | B | 27 | −23.955 | −17.902 | 11.377 | 1.00 | 44.24 | B | N |
| ATOM | 2482 | CA | LYS | B | 27 | −25.140 | −17.854 | 10.539 | 1.00 | 44.91 | B | C |
| ATOM | 2483 | CB | LYS | B | 27 | −26.279 | −18.702 | 11.154 | 1.00 | 47.98 | B | C |
| ATOM | 2484 | CG | LYS | B | 27 | −26.908 | −19.739 | 10.222 | 1.00 | 50.64 | B | C |
| ATOM | 2485 | CD | LYS | B | 27 | −28.321 | −20.163 | 10.639 | 1.00 | 51.72 | B | C |
| ATOM | 2486 | CE | LYS | B | 27 | −28.667 | −21.578 | 10.160 | 1.00 | 50.07 | B | C |
| ATOM | 2487 | NZ | LYS | B | 27 | −30.130 | −21.767 | 9.974 | 1.00 | 48.98 | B | N |
| ATOM | 2488 | C | LYS | B | 27 | −25.533 | −16.389 | 10.501 | 1.00 | 43.09 | B | C |
| ATOM | 2489 | O | LYS | B | 27 | −25.380 | −15.676 | 11.500 | 1.00 | 42.47 | B | O |
| ATOM | 2490 | N | ASP | B | 28 | −26.028 | −15.939 | 9.357 | 1.00 | 40.93 | B | N |
| ATOM | 2491 | CA | ASP | B | 28 | −26.461 | −14.555 | 9.190 | 1.00 | 40.17 | B | C |
| ATOM | 2492 | CB | ASP | B | 28 | −25.400 | −13.747 | 8.407 | 1.00 | 40.04 | B | C |
| ATOM | 2493 | CG | ASP | B | 28 | −25.757 | −12.248 | 8.223 | 1.00 | 39.03 | B | C |
| ATOM | 2494 | OD1 | ASP | B | 28 | −26.943 | −11.879 | 7.999 | 1.00 | 39.21 | B | O |
| ATOM | 2495 | OD2 | ASP | B | 28 | −24.807 | −11.438 | 8.258 | 1.00 | 34.92 | B | O |
| ATOM | 2496 | C | ASP | B | 28 | −27.793 | −14.620 | 8.472 | 1.00 | 39.30 | B | C |
| ATOM | 2497 | O | ASP | B | 28 | −27.863 | −14.812 | 7.260 | 1.00 | 37.02 | B | O |
| ATOM | 2498 | N | THR | B | 29 | −28.863 | −14.460 | 9.234 | 1.00 | 41.34 | B | N |
| ATOM | 2499 | CA | THR | B | 29 | −30.206 | −14.595 | 8.673 | 1.00 | 42.38 | B | C |
| ATOM | 2500 | CB | THR | B | 29 | −31.217 | −15.019 | 9.771 | 1.00 | 42.14 | B | C |
| ATOM | 2501 | OG1 | THR | B | 29 | −31.801 | −13.865 | 10.404 | 1.00 | 41.11 | B | O |
| ATOM | 2502 | CG2 | THR | B | 29 | −30.511 | −15.907 | 10.837 | 1.00 | 41.47 | B | C |
| ATOM | 2503 | C | THR | B | 29 | −30.610 | −13.293 | 7.942 | 1.00 | 42.90 | B | C |
| ATOM | 2504 | O | THR | B | 29 | −31.536 | −13.303 | 7.113 | 1.00 | 41.57 | B | O |
| ATOM | 2505 | N | THR | B | 30 | −29.862 | −12.208 | 8.209 | 1.00 | 42.18 | B | N |
| ATOM | 2506 | CA | THR | B | 30 | −30.243 | −10.849 | 7.803 | 1.00 | 42.26 | B | C |
| ATOM | 2507 | CB | THR | B | 30 | −29.460 | −9.772 | 8.569 | 1.00 | 41.59 | B | C |
| ATOM | 2508 | OG1 | THR | B | 30 | −28.125 | −9.732 | 8.078 | 1.00 | 44.54 | B | O |
| ATOM | 2509 | CG2 | THR | B | 30 | −29.427 | −10.041 | 10.071 | 1.00 | 41.22 | B | C |
| ATOM | 2510 | C | THR | B | 30 | −30.012 | −10.576 | 6.324 | 1.00 | 43.07 | B | C |
| ATOM | 2511 | O | THR | B | 30 | −30.569 | −9.628 | 5.790 | 1.00 | 43.49 | B | O |
| ATOM | 2512 | N | ARG | B | 31 | −29.170 | −11.381 | 5.676 | 1.00 | 43.41 | B | N |
| ATOM | 2513 | CA | ARG | B | 31 | −28.950 | −11.270 | 4.234 | 1.00 | 42.73 | B | C |
| ATOM | 2514 | CB | ARG | B | 31 | −27.457 | −11.104 | 3.947 | 1.00 | 41.54 | B | C |
| ATOM | 2515 | CG | ARG | B | 31 | −26.880 | −9.779 | 4.412 | 1.00 | 40.12 | B | C |
| ATOM | 2516 | CD | ARG | B | 31 | −25.358 | −9.694 | 4.271 | 1.00 | 40.81 | B | C |
| ATOM | 2517 | NE | ARG | B | 31 | −24.850 | −8.404 | 4.746 | 1.00 | 39.64 | B | N |
| ATOM | 2518 | CZ | ARG | B | 31 | −24.620 | −8.104 | 6.025 | 1.00 | 37.51 | B | C |
| ATOM | 2519 | NH1 | ARG | B | 31 | −24.818 | −9.001 | 6.977 | 1.00 | 40.18 | B | N |
| ATOM | 2520 | NH2 | ARG | B | 31 | −24.184 | −6.906 | 6.361 | 1.00 | 34.41 | B | N |
| ATOM | 2521 | C | ARG | B | 31 | −29.492 | −12.510 | 3.521 | 1.00 | 42.99 | B | C |
| ATOM | 2522 | O | ARG | B | 31 | −29.076 | −13.639 | 3.813 | 1.00 | 41.18 | B | O |
| ATOM | 2523 | N | GLY | B | 32 | −30.421 | −12.288 | 2.595 | 1.00 | 44.05 | B | N |
| ATOM | 2524 | CA | GLY | B | 32 | −31.026 | −13.364 | 1.795 | 1.00 | 47.95 | B | C |
| ATOM | 2525 | C | GLY | B | 32 | −31.677 | −14.463 | 2.618 | 1.00 | 49.75 | B | C |
| ATOM | 2526 | O | GLY | B | 32 | −32.181 | −14.216 | 3.717 | 1.00 | 52.02 | B | O |
| ATOM | 2527 | N | GLN | B | 33 | −31.633 | −15.689 | 2.101 | 1.00 | 49.78 | B | N |
| ATOM | 2528 | CA | GLN | B | 33 | −32.066 | −16.860 | 2.865 | 1.00 | 48.09 | B | C |
| ATOM | 2529 | CB | GLN | B | 33 | −32.511 | −17.987 | 1.926 | 1.00 | 49.40 | B | C |
| ATOM | 2530 | CG | GLN | B | 33 | −33.652 | −17.599 | 0.979 | 1.00 | 51.77 | B | C |
| ATOM | 2531 | CD | GLN | B | 33 | −33.573 | −18.279 | −0.391 | 1.00 | 52.73 | B | C |
| ATOM | 2532 | OE1 | GLN | B | 33 | −33.197 | −19.442 | −0.508 | 1.00 | 50.83 | B | O |
| ATOM | 2533 | NE2 | GLN | B | 33 | −33.944 | −17.546 | −1.436 | 1.00 | 56.68 | B | N |
| ATOM | 2534 | C | GLN | B | 33 | −30.966 | −17.360 | 3.803 | 1.00 | 45.74 | B | C |
| ATOM | 2535 | O | GLN | B | 33 | −31.055 | −18.473 | 4.292 | 1.00 | 48.18 | B | O |
| ATOM | 2536 | N | GLY | B | 34 | −29.933 | −16.551 | 4.048 | 1.00 | 44.06 | B | N |
| ATOM | 2537 | CA | GLY | B | 34 | −28.862 | −16.901 | 4.998 | 1.00 | 40.43 | B | C |
| ATOM | 2538 | C | GLY | B | 34 | −27.446 | −16.828 | 4.428 | 1.00 | 37.82 | B | C |
| ATOM | 2539 | O | GLY | B | 34 | −27.265 | −16.890 | 3.210 | 1.00 | 35.60 | B | O |
| ATOM | 2540 | N | ILE | B | 35 | −26.453 | −16.679 | 5.320 | 1.00 | 34.43 | B | N |
| ATOM | 2541 | CA | ILE | B | 35 | −25.030 | −16.796 | 4.984 | 1.00 | 33.25 | B | C |
| ATOM | 2542 | CB | ILE | B | 35 | −24.312 | −15.420 | 4.930 | 1.00 | 37.23 | B | C |
| ATOM | 2543 | CG1 | ILE | B | 35 | −24.686 | −14.664 | 3.653 | 1.00 | 38.63 | B | C |
| ATOM | 2544 | CD1 | ILE | B | 35 | −24.189 | −13.226 | 3.623 | 1.00 | 39.64 | B | C |
| ATOM | 2545 | CG2 | ILE | B | 35 | −22.779 | −15.561 | 4.974 | 1.00 | 37.29 | B | C |
| ATOM | 2546 | C | ILE | B | 35 | −24.372 | −17.650 | 6.052 | 1.00 | 31.28 | B | C |
| ATOM | 2547 | O | ILE | B | 35 | −24.379 | −17.291 | 7.239 | 1.00 | 29.76 | B | O |
| ATOM | 2548 | N | VAL | B | 36 | −23.779 | −18.768 | 5.629 | 1.00 | 30.21 | B | N |
| ATOM | 2549 | CA | VAL | B | 36 | −23.278 | −19.763 | 6.577 | 1.00 | 29.18 | B | C |
| ATOM | 2550 | CB | VAL | B | 36 | −24.183 | −21.024 | 6.652 | 1.00 | 29.87 | B | C |
| ATOM | 2551 | CG1 | VAL | B | 36 | −24.401 | −21.390 | 8.107 | 1.00 | 31.50 | B | C |
| ATOM | 2552 | CG2 | VAL | B | 36 | −25.537 | −20.825 | 5.979 | 1.00 | 29.38 | B | C |
| ATOM | 2553 | C | VAL | B | 36 | −21.869 | −20.237 | 6.267 | 1.00 | 27.11 | B | C |
| ATOM | 2554 | O | VAL | B | 36 | −21.508 | −20.504 | 5.123 | 1.00 | 26.29 | B | O |
| ATOM | 2555 | N | THR | B | 37 | −21.101 | −20.409 | 7.319 | 1.00 | 26.36 | B | N |
| ATOM | 2556 | CA | THR | B | 37 | −19.716 | −20.767 | 7.182 | 1.00 | 27.97 | B | C |
| ATOM | 2557 | CB | THR | B | 37 | −18.851 | −19.545 | 7.550 | 1.00 | 29.05 | B | C |
| ATOM | 2558 | OG1 | THR | B | 37 | −19.158 | −18.474 | 6.637 | 1.00 | 30.25 | B | O |
| ATOM | 2559 | CG2 | THR | B | 37 | −17.335 | −19.865 | 7.538 | 1.00 | 28.48 | B | C |
| ATOM | 2560 | C | THR | B | 37 | −19.367 | −22.015 | 8.026 | 1.00 | 28.89 | B | C |
| ATOM | 2561 | O | THR | B | 37 | −19.527 | −22.015 | 9.258 | 1.00 | 26.39 | B | O |
| ATOM | 2562 | N | TYR | B | 38 | −18.894 | −23.055 | 7.321 | 1.00 | 30.58 | B | N |
| ATOM | 2563 | CA | TYR | B | 38 | −18.515 | −24.352 | 7.890 | 1.00 | 33.15 | B | C |
| ATOM | 2564 | CB | TYR | B | 38 | −19.122 | −25.498 | 7.067 | 1.00 | 33.68 | B | C |
| ATOM | 2565 | CG | TYR | B | 38 | −20.602 | −25.408 | 6.810 | 1.00 | 34.72 | B | C |
| ATOM | 2566 | CD1 | TYR | B | 38 | −21.101 | −24.645 | 5.751 | 1.00 | 33.58 | B | C |
| ATOM | 2567 | CE1 | TYR | B | 38 | −22.459 | −24.560 | 5.503 | 1.00 | 33.35 | B | C |
| ATOM | 2568 | CZ | TYR | B | 38 | −23.338 | −25.250 | 6.307 | 1.00 | 33.94 | B | C |
| ATOM | 2569 | OH | TYR | B | 38 | −24.675 | −25.190 | 6.053 | 1.00 | 32.94 | B | O |
| ATOM | 2570 | CE2 | TYR | B | 38 | −22.875 | −26.007 | 7.369 | 1.00 | 36.31 | B | C |
| ATOM | 2571 | CD2 | TYR | B | 38 | −21.509 | −26.091 | 7.610 | 1.00 | 36.06 | B | C |
| ATOM | 2572 | C | TYR | B | 38 | −17.006 | −24.557 | 7.858 | 1.00 | 33.86 | B | C |
| ATOM | 2573 | O | TYR | B | 38 | −16.284 | −23.768 | 7.244 | 1.00 | 35.29 | B | O |
| ATOM | 2574 | N | SER | B | 39 | −16.555 | −25.648 | 8.483 | 1.00 | 32.45 | B | N |
| ATOM | 2575 | CA | SER | B | 39 | −15.151 | −26.094 | 8.447 | 1.00 | 31.81 | B | C |
| ATOM | 2576 | CB | SER | B | 39 | −14.480 | −25.943 | 9.826 | 1.00 | 30.22 | B | C |
| ATOM | 2577 | OG | SER | B | 39 | −13.271 | −26.688 | 9.939 | 1.00 | 28.69 | B | O |
| ATOM | 2578 | C | SER | B | 39 | −15.130 | −27.560 | 8.023 | 1.00 | 32.86 | B | C |
| ATOM | 2579 | O | SER | B | 39 | −15.972 | −28.338 | 8.481 | 1.00 | 32.92 | B | O |
| ATOM | 2580 | N | ALA | B | 40 | −14.171 | −27.940 | 7.169 | 1.00 | 31.62 | B | N |
| ATOM | 2581 | CA | ALA | B | 40 | −14.112 | −29.305 | 6.650 | 1.00 | 30.64 | B | C |
| ATOM | 2582 | CB | ALA | B | 40 | −13.763 | −29.298 | 5.174 | 1.00 | 29.75 | B | C |
| ATOM | 2583 | C | ALA | B | 40 | −13.130 | −30.173 | 7.436 | 1.00 | 31.48 | B | C |
| ATOM | 2584 | O | ALA | B | 40 | −12.928 | −31.349 | 7.097 | 1.00 | 29.96 | B | O |
| ATOM | 2585 | N | GLY | B | 41 | −12.535 | −29.603 | 8.487 | 1.00 | 31.27 | B | N |
| ATOM | 2586 | CA | GLY | B | 41 | −11.567 | −30.329 | 9.308 | 1.00 | 32.58 | B | C |
| ATOM | 2587 | C | GLY | B | 41 | −10.464 | −30.911 | 8.448 | 1.00 | 34.88 | B | C |
| ATOM | 2588 | O | GLY | B | 41 | −10.005 | −32.016 | 8.681 | 1.00 | 41.21 | B | O |
| ATOM | 2589 | N | ASN | B | 42 | −10.055 | −30.164 | 7.432 | 1.00 | 35.41 | B | N |
| ATOM | 2590 | CA | ASN | B | 42 | −8.963 | −30.556 | 6.542 | 1.00 | 35.57 | B | C |
| ATOM | 2591 | CB | ASN | B | 42 | −7.626 | −30.565 | 7.290 | 1.00 | 35.12 | B | C |
| ATOM | 2592 | CG | ASN | B | 42 | −7.341 | −29.234 | 7.988 | 1.00 | 35.46 | B | C |
| ATOM | 2593 | OD1 | ASN | B | 42 | −6.975 | −29.225 | 9.143 | 1.00 | 37.01 | B | O |
| ATOM | 2594 | ND2 | ASN | B | 42 | −7.508 | −28.109 | 7.281 | 1.00 | 35.13 | B | N |
| ATOM | 2595 | C | ASN | B | 42 | −9.235 | −31.851 | 5.820 | 1.00 | 35.04 | B | C |
| ATOM | 2596 | O | ASN | B | 42 | −8.323 | −32.620 | 5.567 | 1.00 | 31.81 | B | O |
| ATOM | 2597 | N | ARG | B | 43 | −10.501 | −32.017 | 5.426 | 1.00 | 39.05 | B | N |
| ATOM | 2598 | CA | ARG | B | 43 | −11.015 | −33.222 | 4.776 | 1.00 | 43.00 | B | C |
| ATOM | 2599 | CB | ARG | B | 43 | −11.892 | −33.984 | 5.775 | 1.00 | 48.73 | B | C |
| ATOM | 2600 | CG | ARG | B | 43 | −11.097 | −34.876 | 6.718 | 1.00 | 53.88 | B | C |
| ATOM | 2601 | CD | ARG | B | 43 | −10.656 | −36.140 | 6.001 | 1.00 | 59.88 | B | C |
| ATOM | 2602 | NE | ARG | B | 43 | −10.497 | −37.266 | 6.925 | 1.00 | 71.15 | B | N |
| ATOM | 2603 | CZ | ARG | B | 43 | −11.497 | −37.925 | 7.523 | 1.00 | 72.97 | B | C |
| ATOM | 2604 | NH1 | ARG | B | 43 | −12.772 | −37.577 | 7.324 | 1.00 | 70.94 | B | N |
| ATOM | 2605 | NH2 | ARG | B | 43 | −11.216 | −38.937 | 8.346 | 1.00 | 74.66 | B | N |
| ATOM | 2606 | C | ARG | B | 43 | −11.806 | −32.894 | 3.521 | 1.00 | 40.05 | B | C |
| ATOM | 2607 | O | ARG | B | 43 | −11.901 | −31.731 | 3.132 | 1.00 | 42.57 | B | O |
| ATOM | 2608 | N | SER | B | 44 | −12.381 | −33.915 | 2.894 | 1.00 | 38.32 | B | N |
| ATOM | 2609 | CA | SER | B | 44 | −12.999 | −33.759 | 1.574 | 1.00 | 39.47 | B | C |
| ATOM | 2610 | CB | SER | B | 44 | −12.712 | −34.982 | 0.695 | 1.00 | 40.23 | B | C |
| ATOM | 2611 | OG | SER | B | 44 | −11.445 | −34.869 | 0.078 | 1.00 | 42.89 | B | O |
| ATOM | 2612 | C | SER | B | 44 | −14.501 | −33.503 | 1.554 | 1.00 | 37.25 | B | C |
| ATOM | 2613 | O | SER | B | 44 | −15.006 | −32.835 | 0.655 | 1.00 | 36.44 | B | O |
| ATOM | 2614 | N | SER | B | 45 | −15.244 | −34.065 | 2.486 | 1.00 | 36.28 | B | N |
| ATOM | 2615 | CA | SER | B | 45 | −16.688 | −34.005 | 2.324 | 1.00 | 36.90 | B | C |
| ATOM | 2616 | CB | SER | B | 45 | −17.413 | −35.089 | 3.144 | 1.00 | 36.36 | B | C |
| ATOM | 2617 | OG | SER | B | 45 | −17.285 | −34.843 | 4.525 | 1.00 | 36.97 | B | O |
| ATOM | 2618 | C | SER | B | 45 | −17.190 | −32.595 | 2.645 | 1.00 | 36.54 | B | C |
| ATOM | 2619 | O | SER | B | 45 | −16.577 | −31.857 | 3.420 | 1.00 | 33.48 | B | O |
| ATOM | 2620 | N | LEU | B | 46 | −18.304 | −32.246 | 2.012 | 1.00 | 37.13 | B | N |
| ATOM | 2621 | CA | LEU | B | 46 | −18.929 | −30.944 | 2.147 | 1.00 | 39.27 | B | C |
| ATOM | 2622 | CB | LEU | B | 46 | −18.853 | −30.188 | 0.811 | 1.00 | 38.08 | B | C |
| ATOM | 2623 | CG | LEU | B | 46 | −17.468 | −30.074 | 0.156 | 1.00 | 36.84 | B | C |
| ATOM | 2624 | CD1 | LEU | B | 46 | −17.606 | −29.703 | −1.326 | 1.00 | 35.76 | B | C |
| ATOM | 2625 | CD2 | LEU | B | 46 | −16.578 | −29.092 | 0.925 | 1.00 | 35.95 | B | C |
| ATOM | 2626 | C | LEU | B | 46 | −20.399 | −31.131 | 2.546 | 1.00 | 41.41 | B | C |
| ATOM | 2627 | O | LEU | B | 46 | −20.968 | −32.200 | 2.316 | 1.00 | 44.36 | B | O |
| ATOM | 2628 | N | PRO | B | 47 | −21.020 | −30.093 | 3.130 | 1.00 | 39.28 | B | N |
| ATOM | 2629 | CA | PRO | B | 47 | −20.411 | −28.820 | 3.520 | 1.00 | 37.84 | B | C |
| ATOM | 2630 | CB | PRO | B | 47 | −21.611 | −28.006 | 4.011 | 1.00 | 40.67 | B | C |
| ATOM | 2631 | CG | PRO | B | 47 | −22.644 | −29.037 | 4.374 | 1.00 | 40.15 | B | C |
| ATOM | 2632 | CD | PRO | B | 47 | −22.479 | −30.061 | 3.306 | 1.00 | 38.74 | B | C |
| ATOM | 2633 | C | PRO | B | 47 | −19.403 | −28.941 | 4.649 | 1.00 | 35.08 | B | C |
| ATOM | 2634 | O | PRO | B | 47 | −18.610 | −28.044 | 4.831 | 1.00 | 32.64 | B | O |
| ATOM | 2635 | N | GLY | B | 48 | −19.438 | −30.045 | 5.390 | 1.00 | 33.92 | B | N |
| ATOM | 2636 | CA | GLY | B | 48 | −18.651 | −30.180 | 6.603 | 1.00 | 32.39 | B | C |
| ATOM | 2637 | C | GLY | B | 48 | −19.466 | −29.671 | 7.770 | 1.00 | 33.27 | B | C |
| ATOM | 2638 | O | GLY | B | 48 | −20.677 | −29.476 | 7.644 | 1.00 | 33.45 | B | O |
| ATOM | 2639 | N | THR | B | 49 | −18.790 | −29.432 | 8.892 | 1.00 | 34.94 | B | N |
| ATOM | 2640 | CA | THR | B | 49 | −19.414 | −28.958 | 10.130 | 1.00 | 38.98 | B | C |
| ATOM | 2641 | CB | THR | B | 49 | −18.513 | −29.273 | 11.339 | 1.00 | 39.73 | B | C |
| ATOM | 2642 | OG1 | THR | B | 49 | −18.099 | −30.641 | 11.273 | 1.00 | 40.95 | B | O |
| ATOM | 2643 | CG2 | THR | B | 49 | −19.242 | −29.015 | 12.663 | 1.00 | 39.97 | B | C |
| ATOM | 2644 | C | THR | B | 49 | −19.720 | −27.444 | 10.184 | 1.00 | 42.89 | B | C |
| ATOM | 2645 | O | THR | B | 49 | −18.880 | −26.602 | 9.868 | 1.00 | 40.45 | B | O |
| ATOM | 2646 | N | LEU | B | 50 | −20.933 | −27.114 | 10.623 | 1.00 | 48.49 | B | N |
| ATOM | 2647 | CA | LEU | B | 50 | −21.292 | −25.732 | 10.920 | 1.00 | 48.73 | B | C |
| ATOM | 2648 | CB | LEU | B | 50 | −22.795 | −25.610 | 11.146 | 1.00 | 49.85 | B | C |
| ATOM | 2649 | CG | LEU | B | 50 | −23.368 | −24.241 | 11.494 | 1.00 | 51.48 | B | C |
| ATOM | 2650 | CD1 | LEU | B | 50 | −22.680 | −23.122 | 10.722 | 1.00 | 52.59 | B | C |
| ATOM | 2651 | CD2 | LEU | B | 50 | −24.875 | −24.227 | 11.229 | 1.00 | 52.22 | B | C |
| ATOM | 2652 | C | LEU | B | 50 | −20.518 | −25.298 | 12.157 | 1.00 | 47.59 | B | C |
| ATOM | 2653 | O | LEU | B | 50 | −20.480 | −26.016 | 13.158 | 1.00 | 49.60 | B | O |
| ATOM | 2654 | N | LEU | B | 51 | −19.877 | −24.138 | 12.057 | 1.00 | 46.71 | B | N |
| ATOM | 2655 | CA | LEU | B | 51 | −18.968 | −23.635 | 13.080 | 1.00 | 43.84 | B | C |
| ATOM | 2656 | CB | LEU | B | 51 | −17.950 | −22.670 | 12.451 | 1.00 | 45.70 | B | C |
| ATOM | 2657 | CG | LEU | B | 51 | −16.529 | −23.222 | 12.293 | 1.00 | 46.99 | B | C |
| ATOM | 2658 | CD1 | LEU | B | 51 | −15.996 | −22.783 | 10.946 | 1.00 | 47.24 | B | C |
| ATOM | 2659 | CD2 | LEU | B | 51 | −15.613 | −22.792 | 13.454 | 1.00 | 46.81 | B | C |
| ATOM | 2660 | C | LEU | B | 51 | −19.690 | −22.965 | 14.244 | 1.00 | 41.22 | B | C |
| ATOM | 2661 | O | LEU | B | 51 | −20.753 | −22.352 | 14.083 | 1.00 | 37.73 | B | O |
| ATOM | 2662 | N | THR | B | 52 | −19.043 | −23.038 | 15.403 | 1.00 | 40.19 | B | N |
| ATOM | 2663 | CA | THR | B | 52 | −19.694 | −22.874 | 16.699 | 1.00 | 38.68 | B | C |
| ATOM | 2664 | CB | THR | B | 52 | −20.030 | −24.303 | 17.224 | 1.00 | 37.51 | B | C |
| ATOM | 2665 | OG1 | THR | B | 52 | −21.182 | −24.776 | 16.532 | 1.00 | 34.76 | B | O |
| ATOM | 2666 | CG2 | THR | B | 52 | −20.290 | −24.379 | 18.732 | 1.00 | 37.73 | B | C |
| ATOM | 2667 | C | THR | B | 52 | −18.788 | −22.103 | 17.672 | 1.00 | 37.74 | B | C |
| ATOM | 2668 | O | THR | B | 52 | −17.577 | −22.245 | 17.639 | 1.00 | 39.97 | B | O |
| ATOM | 2669 | N | SER | B | 53 | −19.371 | −21.279 | 18.529 | 1.00 | 39.22 | B | N |
| ATOM | 2670 | CA | SER | B | 53 | −18.636 | −20.722 | 19.673 | 1.00 | 41.33 | B | C |
| ATOM | 2671 | CB | SER | B | 53 | −17.766 | −19.518 | 19.263 | 1.00 | 40.16 | B | C |
| ATOM | 2672 | OG | SER | B | 53 | −17.139 | −18.905 | 20.382 | 1.00 | 37.49 | B | O |
| ATOM | 2673 | C | SER | B | 53 | −19.643 | −20.307 | 20.739 | 1.00 | 45.00 | B | C |
| ATOM | 2674 | O | SER | B | 53 | −20.589 | −19.556 | 20.450 | 1.00 | 46.25 | B | O |
| ATOM | 2675 | N | SER | B | 54 | −19.461 | −20.823 | 21.955 | 1.00 | 46.91 | B | N |
| ATOM | 2676 | CA | SER | B | 54 | −20.263 | −20.398 | 23.104 | 1.00 | 47.89 | B | C |
| ATOM | 2677 | CB | SER | B | 54 | −20.046 | −21.350 | 24.292 | 1.00 | 47.12 | B | C |
| ATOM | 2678 | OG | SER | B | 54 | −18.666 | −21.531 | 24.586 | 1.00 | 46.27 | B | O |
| ATOM | 2679 | C | SER | B | 54 | −19.953 | −18.928 | 23.478 | 1.00 | 48.96 | B | C |
| ATOM | 2680 | O | SER | B | 54 | −20.833 | −18.202 | 23.926 | 1.00 | 49.21 | B | O |
| ATOM | 2681 | N | SER | B | 55 | −18.718 | −18.493 | 23.219 | 1.00 | 47.87 | B | N |
| ATOM | 2682 | CA | SER | B | 55 | −18.222 | −17.161 | 23.587 | 1.00 | 44.68 | B | C |
| ATOM | 2683 | CB | SER | B | 55 | −16.685 | −17.209 | 23.560 | 1.00 | 44.59 | B | C |
| ATOM | 2684 | OG | SER | B | 55 | −16.106 | −16.112 | 24.240 | 1.00 | 44.52 | B | O |
| ATOM | 2685 | C | SER | B | 55 | −18.707 | −15.986 | 22.695 | 1.00 | 44.27 | B | C |
| ATOM | 2686 | O | SER | B | 55 | −18.553 | −14.809 | 23.068 | 1.00 | 44.09 | B | O |
| ATOM | 2687 | N | ASN | B | 56 | −19.279 | −16.300 | 21.526 | 1.00 | 42.16 | B | N |
| ATOM | 2688 | CA | ASN | B | 56 | −19.411 | −15.344 | 20.385 | 1.00 | 38.46 | B | C |
| ATOM | 2689 | CB | ASN | B | 56 | −20.558 | −14.340 | 20.601 | 1.00 | 35.65 | B | C |
| ATOM | 2690 | CG | ASN | B | 56 | −21.226 | −13.902 | 19.290 | 1.00 | 34.01 | B | C |
| ATOM | 2691 | OD1 | ASN | B | 56 | −21.996 | −12.952 | 19.283 | 1.00 | 36.17 | B | O |
| ATOM | 2692 | ND2 | ASN | B | 56 | −20.947 | −14.590 | 18.194 | 1.00 | 30.20 | B | N |
| ATOM | 2693 | C | ASN | B | 56 | −18.101 | −14.601 | 19.985 | 1.00 | 38.13 | B | C |
| ATOM | 2694 | O | ASN | B | 56 | −18.137 | −13.659 | 19.202 | 1.00 | 35.54 | B | O |
| ATOM | 2695 | N | ILE | B | 57 | −16.967 | −15.032 | 20.536 | 1.00 | 38.44 | B | N |
| ATOM | 2696 | CA | ILE | B | 57 | −15.654 | −14.668 | 20.039 | 1.00 | 42.11 | B | C |
| ATOM | 2697 | CB | ILE | B | 57 | −14.683 | −14.350 | 21.189 | 1.00 | 42.20 | B | C |
| ATOM | 2698 | CG1 | ILE | B | 57 | −15.350 | −13.491 | 22.273 | 1.00 | 44.11 | B | C |
| ATOM | 2699 | CD1 | ILE | B | 57 | −15.809 | −12.115 | 21.839 | 1.00 | 44.60 | B | C |
| ATOM | 2700 | CG2 | ILE | B | 57 | −13.437 | −13.665 | 20.654 | 1.00 | 41.76 | B | C |
| ATOM | 2701 | C | ILE | B | 57 | −15.113 | −15.873 | 19.234 | 1.00 | 46.06 | B | C |
| ATOM | 2702 | O | ILE | B | 57 | −15.270 | −17.026 | 19.669 | 1.00 | 50.20 | B | O |
| ATOM | 2703 | N | TRP | B | 58 | −14.475 | −15.607 | 18.085 | 1.00 | 43.11 | B | N |
| ATOM | 2704 | CA | TRP | B | 58 | −14.066 | −16.648 | 17.136 | 1.00 | 40.89 | B | C |
| ATOM | 2705 | CB | TRP | B | 58 | −14.801 | −16.457 | 15.834 | 1.00 | 40.59 | B | C |
| ATOM | 2706 | CG | TRP | B | 58 | −16.245 | −16.402 | 16.008 | 1.00 | 40.53 | B | C |
| ATOM | 2707 | CD1 | TRP | B | 58 | −16.962 | −15.345 | 16.414 | 1.00 | 40.30 | B | C |
| ATOM | 2708 | NE1 | TRP | B | 58 | −18.288 | −15.660 | 16.467 | 1.00 | 40.67 | B | N |
| ATOM | 2709 | CE2 | TRP | B | 58 | −18.449 | −16.960 | 16.090 | 1.00 | 41.82 | B | C |
| ATOM | 2710 | CD2 | TRP | B | 58 | −17.168 | −17.463 | 15.793 | 1.00 | 43.03 | B | C |
| ATOM | 2711 | CE3 | TRP | B | 58 | −17.038 | −18.798 | 15.378 | 1.00 | 44.52 | B | C |
| ATOM | 2712 | CZ3 | TRP | B | 58 | −18.194 | −19.573 | 15.257 | 1.00 | 46.89 | B | C |
| ATOM | 2713 | CH2 | TRP | B | 58 | −19.466 | −19.033 | 15.563 | 1.00 | 46.00 | B | C |
| ATOM | 2714 | CZ2 | TRP | B | 58 | −19.608 | −17.734 | 15.987 | 1.00 | 43.73 | B | C |
| ATOM | 2715 | C | TRP | B | 58 | −12.583 | −16.598 | 16.838 | 1.00 | 43.33 | B | C |
| ATOM | 2716 | O | TRP | B | 58 | −12.073 | −15.572 | 16.390 | 1.00 | 41.28 | B | O |
| ATOM | 2717 | N | ASN | B | 59 | −11.893 | −17.720 | 17.041 | 1.00 | 46.24 | B | N |
| ATOM | 2718 | CA | ASN | B | 59 | −10.441 | −17.752 | 16.912 | 1.00 | 45.70 | B | C |
| ATOM | 2719 | CB | ASN | B | 59 | −9.830 | −18.319 | 18.178 | 1.00 | 49.90 | B | C |
| ATOM | 2720 | CG | ASN | B | 59 | −9.910 | −17.334 | 19.326 | 1.00 | 57.71 | B | C |
| ATOM | 2721 | OD1 | ASN | B | 59 | −9.071 | −16.430 | 19.449 | 1.00 | 61.14 | B | O |
| ATOM | 2722 | ND2 | ASN | B | 59 | −10.934 | −17.481 | 20.165 | 1.00 | 62.09 | B | N |
| ATOM | 2723 | C | ASN | B | 59 | −9.972 | −18.509 | 15.696 | 1.00 | 42.04 | B | C |
| ATOM | 2724 | O | ASN | B | 59 | −8.950 | −19.169 | 15.726 | 1.00 | 45.30 | B | O |
| ATOM | 2725 | N | ASP | B | 60 | −10.707 | −18.369 | 14.609 | 1.00 | 37.51 | B | N |
| ATOM | 2726 | CA | ASP | B | 60 | −10.363 | −19.023 | 13.371 | 1.00 | 36.63 | B | C |
| ATOM | 2727 | CB | ASP | B | 60 | −11.337 | −20.170 | 13.119 | 1.00 | 37.97 | B | C |
| ATOM | 2728 | CG | ASP | B | 60 | −11.017 | −20.941 | 11.861 | 1.00 | 36.97 | B | C |
| ATOM | 2729 | OD1 | ASP | B | 60 | −9.899 | −20.784 | 11.310 | 1.00 | 35.18 | B | O |
| ATOM | 2730 | OD2 | ASP | B | 60 | −11.902 | −21.710 | 11.429 | 1.00 | 37.14 | B | O |
| ATOM | 2731 | C | ASP | B | 60 | −10.381 | −18.016 | 12.211 | 1.00 | 35.22 | B | C |
| ATOM | 2732 | O | ASP | B | 60 | −11.461 | −17.568 | 11.745 | 1.00 | 33.57 | B | O |
| ATOM | 2733 | N | GLY | B | 61 | −9.172 | −17.694 | 11.746 | 1.00 | 31.50 | B | N |
| ATOM | 2734 | CA | GLY | B | 61 | −8.947 | −16.600 | 10.815 | 1.00 | 29.00 | B | C |
| ATOM | 2735 | C | GLY | B | 61 | −9.628 | −16.829 | 9.495 | 1.00 | 28.50 | B | C |
| ATOM | 2736 | O | GLY | B | 61 | −10.389 | −15.978 | 9.025 | 1.00 | 29.45 | B | O |
| ATOM | 2737 | N | ALA | B | 62 | −9.380 | −17.980 | 8.886 | 1.00 | 27.01 | B | N |
| ATOM | 2738 | CA | ALA | B | 62 | −9.980 | −18.248 | 7.581 | 1.00 | 26.15 | B | C |
| ATOM | 2739 | CB | ALA | B | 62 | −9.484 | −19.558 | 7.008 | 1.00 | 27.22 | B | C |
| ATOM | 2740 | C | ALA | B | 62 | −11.494 | −18.232 | 7.642 | 1.00 | 25.10 | B | C |
| ATOM | 2741 | O | ALA | B | 62 | −12.121 | −17.707 | 6.755 | 1.00 | 25.58 | B | O |
| ATOM | 2742 | N | ALA | B | 63 | −12.090 | −18.777 | 8.695 | 1.00 | 25.77 | B | N |
| ATOM | 2743 | CA | ALA | B | 63 | −13.560 | −18.757 | 8.810 | 1.00 | 27.21 | B | C |
| ATOM | 2744 | CB | ALA | B | 63 | −14.029 | −19.602 | 9.989 | 1.00 | 27.02 | B | C |
| ATOM | 2745 | C | ALA | B | 63 | −14.118 | −17.325 | 8.934 | 1.00 | 27.71 | B | C |
| ATOM | 2746 | O | ALA | B | 63 | −15.165 | −17.007 | 8.343 | 1.00 | 27.87 | B | O |
| ATOM | 2747 | N | VAL | B | 64 | −13.417 | −16.488 | 9.712 | 1.00 | 26.10 | B | N |
| ATOM | 2748 | CA | VAL | B | 64 | −13.806 | −15.112 | 9.932 | 1.00 | 24.19 | B | C |
| ATOM | 2749 | CB | VAL | B | 64 | −12.831 | −14.437 | 10.882 | 1.00 | 24.11 | B | C |
| ATOM | 2750 | CG1 | VAL | B | 64 | −13.003 | −12.926 | 10.833 | 1.00 | 24.36 | B | C |
| ATOM | 2751 | CG2 | VAL | B | 64 | −13.047 | −14.965 | 12.292 | 1.00 | 24.69 | B | C |
| ATOM | 2752 | C | VAL | B | 64 | −13.819 | −14.352 | 8.619 | 1.00 | 24.48 | B | C |
| ATOM | 2753 | O | VAL | B | 64 | −14.837 | −13.747 | 8.233 | 1.00 | 23.08 | B | O |
| ATOM | 2754 | N | ASP | B | 65 | −12.687 | −14.386 | 7.924 | 1.00 | 24.36 | B | N |
| ATOM | 2755 | CA | ASP | B | 65 | −12.597 | −13.691 | 6.657 | 1.00 | 24.27 | B | C |
| ATOM | 2756 | CB | ASP | B | 65 | −11.194 | −13.785 | 6.053 | 1.00 | 24.29 | B | C |
| ATOM | 2757 | CG | ASP | B | 65 | −10.179 | −12.899 | 6.778 | 1.00 | 24.48 | B | C |
| ATOM | 2758 | OD1 | ASP | B | 65 | −10.541 | −11.803 | 7.209 | 1.00 | 23.48 | B | O |
| ATOM | 2759 | OD2 | ASP | B | 65 | −9.004 | −13.284 | 6.922 | 1.00 | 25.28 | B | O |
| ATOM | 2760 | C | ASP | B | 65 | −13.650 | −14.266 | 5.731 | 1.00 | 24.81 | B | C |
| ATOM | 2761 | O | ASP | B | 65 | −14.431 | −13.507 | 5.130 | 1.00 | 24.32 | B | O |
| ATOM | 2762 | N | ALA | B | 66 | −13.720 | −15.597 | 5.660 | 1.00 | 24.42 | B | N |
| ATOM | 2763 | CA | ALA | B | 66 | −14.745 | −16.245 | 4.827 | 1.00 | 26.00 | B | C |
| ATOM | 2764 | CB | ALA | B | 66 | −14.748 | −17.751 | 5.038 | 1.00 | 26.76 | B | C |
| ATOM | 2765 | C | ALA | B | 66 | −16.143 | −15.693 | 5.096 | 1.00 | 25.63 | B | C |
| ATOM | 2766 | O | ALA | B | 66 | −16.880 | −15.319 | 4.181 | 1.00 | 22.75 | B | O |
| ATOM | 2767 | N | HIS | B | 67 | −16.480 | −15.650 | 6.375 | 1.00 | 27.71 | B | N |
| ATOM | 2768 | CA | HIS | B | 67 | −17.789 | −15.198 | 6.817 | 1.00 | 30.45 | B | C |
| ATOM | 2769 | CB | HIS | B | 67 | −17.945 | −15.490 | 8.305 | 1.00 | 32.99 | B | C |
| ATOM | 2770 | CG | HIS | B | 67 | −19.353 | −15.419 | 8.779 | 1.00 | 34.84 | B | C |
| ATOM | 2771 | ND1 | HIS | B | 67 | −20.373 | −16.117 | 8.170 | 1.00 | 36.52 | B | N |
| ATOM | 2772 | CE1 | HIS | B | 67 | −21.509 | −15.850 | 8.785 | 1.00 | 37.20 | B | C |
| ATOM | 2773 | NE2 | HIS | B | 67 | −21.257 | −15.016 | 9.778 | 1.00 | 36.99 | B | N |
| ATOM | 2774 | CD2 | HIS | B | 67 | −19.916 | −14.729 | 9.794 | 1.00 | 34.94 | B | C |
| ATOM | 2775 | C | HIS | B | 67 | −18.026 | −13.710 | 6.536 | 1.00 | 29.77 | B | C |
| ATOM | 2776 | O | HIS | B | 67 | −18.971 | −13.350 | 5.823 | 1.00 | 28.30 | B | O |
| ATOM | 2777 | N | ALA | B | 68 | −17.159 | −12.853 | 7.076 | 1.00 | 29.76 | B | N |
| ATOM | 2778 | CA | ALA | B | 68 | −17.281 | −11.398 | 6.850 | 1.00 | 30.76 | B | C |
| ATOM | 2779 | CB | ALA | B | 68 | −16.138 | −10.632 | 7.523 | 1.00 | 30.67 | B | C |
| ATOM | 2780 | C | ALA | B | 68 | −17.320 | −11.067 | 5.362 | 1.00 | 29.91 | B | C |
| ATOM | 2781 | O | ALA | B | 68 | −18.336 | −10.605 | 4.842 | 1.00 | 29.77 | B | O |
| ATOM | 2782 | N | TYR | B | 69 | −16.217 | −11.347 | 4.676 | 1.00 | 27.97 | B | N |
| ATOM | 2783 | CA | TYR | B | 69 | −16.103 | −10.975 | 3.281 | 1.00 | 26.15 | B | C |
| ATOM | 2784 | CB | TYR | B | 69 | −14.755 | −11.400 | 2.656 | 1.00 | 25.06 | B | C |
| ATOM | 2785 | CG | TYR | B | 69 | −13.523 | −10.767 | 3.264 | 1.00 | 23.20 | B | C |
| ATOM | 2786 | CD1 | TYR | B | 69 | −13.542 | −9.468 | 3.784 | 1.00 | 22.72 | B | C |
| ATOM | 2787 | CE1 | TYR | B | 69 | −12.406 | −8.906 | 4.356 | 1.00 | 21.86 | B | C |
| ATOM | 2788 | CZ | TYR | B | 69 | −11.242 | −9.635 | 4.396 | 1.00 | 21.61 | B | C |
| ATOM | 2789 | OH | TYR | B | 69 | −10.096 | −9.115 | 4.939 | 1.00 | 20.56 | B | O |
| ATOM | 2790 | CE2 | TYR | B | 69 | −11.214 | −10.921 | 3.894 | 1.00 | 21.81 | B | C |
| ATOM | 2791 | CD2 | TYR | B | 69 | −12.343 | −11.472 | 3.329 | 1.00 | 22.03 | B | C |
| ATOM | 2792 | C | TYR | B | 69 | −17.240 | −11.529 | 2.460 | 1.00 | 25.31 | B | C |
| ATOM | 2793 | O | TYR | B | 69 | −17.527 | −11.003 | 1.400 | 1.00 | 25.46 | B | O |
| ATOM | 2794 | N | THR | B | 70 | −17.899 | −12.582 | 2.916 | 1.00 | 27.08 | B | N |
| ATOM | 2795 | CA | THR | B | 70 | −19.056 | −13.065 | 2.145 | 1.00 | 28.79 | B | C |
| ATOM | 2796 | CB | THR | B | 70 | −19.421 | −14.549 | 2.426 | 1.00 | 29.13 | B | C |
| ATOM | 2797 | OG1 | THR | B | 70 | −18.350 | −15.412 | 2.011 | 1.00 | 28.17 | B | O |
| ATOM | 2798 | CG2 | THR | B | 70 | −20.666 | −14.947 | 1.640 | 1.00 | 30.19 | B | C |
| ATOM | 2799 | C | THR | B | 70 | −20.236 | −12.077 | 2.332 | 1.00 | 28.56 | B | C |
| ATOM | 2800 | O | THR | B | 70 | −20.896 | −11.713 | 1.345 | 1.00 | 26.58 | B | O |
| ATOM | 2801 | N | ALA | B | 71 | −20.457 | −11.607 | 3.569 | 1.00 | 28.13 | B | N |
| ATOM | 2802 | CA | ALA | B | 71 | −21.405 | −10.506 | 3.805 | 1.00 | 27.78 | B | C |
| ATOM | 2803 | CB | ALA | B | 71 | −21.412 | −10.101 | 5.261 | 1.00 | 26.69 | B | C |
| ATOM | 2804 | C | ALA | B | 71 | −21.075 | −9.293 | 2.922 | 1.00 | 28.42 | B | C |
| ATOM | 2805 | O | ALA | B | 71 | −21.969 | −8.680 | 2.317 | 1.00 | 27.67 | B | O |
| ATOM | 2806 | N | LYS | B | 72 | −19.789 | −8.964 | 2.840 | 1.00 | 29.66 | B | N |
| ATOM | 2807 | CA | LYS | B | 72 | −19.346 | −7.772 | 2.133 | 1.00 | 31.07 | B | C |
| ATOM | 2808 | CB | LYS | B | 72 | −17.842 | −7.590 | 2.277 | 1.00 | 32.79 | B | C |
| ATOM | 2809 | CG | LYS | B | 72 | −17.258 | −6.408 | 1.512 | 1.00 | 35.11 | B | C |
| ATOM | 2810 | CD | LYS | B | 72 | −15.747 | −6.289 | 1.716 | 1.00 | 36.65 | B | C |
| ATOM | 2811 | CE | LYS | B | 72 | −15.242 | −4.933 | 1.257 | 1.00 | 38.51 | B | C |
| ATOM | 2812 | NZ | LYS | B | 72 | −13.761 | −4.866 | 1.344 | 1.00 | 40.21 | B | N |
| ATOM | 2813 | C | LYS | B | 72 | −19.717 | −7.872 | 0.677 | 1.00 | 32.30 | B | C |
| ATOM | 2814 | O | LYS | B | 72 | −20.162 | −6.889 | 0.079 | 1.00 | 35.50 | B | O |
| ATOM | 2815 | N | VAL | B | 73 | −19.546 | −9.066 | 0.112 | 1.00 | 32.14 | B | N |
| ATOM | 2816 | CA | VAL | B | 73 | −19.814 | −9.304 | −1.320 | 1.00 | 30.81 | B | C |
| ATOM | 2817 | CB | VAL | B | 73 | −19.131 | −10.611 | −1.824 | 1.00 | 29.58 | B | C |
| ATOM | 2818 | CG1 | VAL | B | 73 | −19.415 | −10.861 | −3.297 | 1.00 | 27.79 | B | C |
| ATOM | 2819 | CG2 | VAL | B | 73 | −17.620 | −10.558 | −1.574 | 1.00 | 30.18 | B | C |
| ATOM | 2820 | C | VAL | B | 73 | −21.312 | −9.362 | −1.576 | 1.00 | 30.15 | B | C |
| ATOM | 2821 | O | VAL | B | 73 | −21.756 | −9.011 | −2.646 | 1.00 | 28.69 | B | O |
| ATOM | 2822 | N | TYR | B | 74 | −22.075 | −9.825 | −0.587 | 1.00 | 32.57 | B | N |
| ATOM | 2823 | CA | TYR | B | 74 | −23.525 | −9.800 | −0.654 | 1.00 | 34.78 | B | C |
| ATOM | 2824 | CB | TYR | B | 74 | −24.141 | −10.527 | 0.543 | 1.00 | 38.44 | B | C |
| ATOM | 2825 | CG | TYR | B | 74 | −25.653 | −10.515 | 0.518 | 1.00 | 42.42 | B | C |
| ATOM | 2826 | CD1 | TYR | B | 74 | −26.367 | −9.478 | 1.108 | 1.00 | 45.08 | B | C |
| ATOM | 2827 | CE1 | TYR | B | 74 | −27.747 | −9.452 | 1.072 | 1.00 | 46.85 | B | C |
| ATOM | 2828 | CZ | TYR | B | 74 | −28.423 | −10.461 | 0.432 | 1.00 | 46.58 | B | C |
| ATOM | 2829 | OH | TYR | B | 74 | −29.792 | −10.430 | 0.400 | 1.00 | 48.77 | B | O |
| ATOM | 2830 | CE2 | TYR | B | 74 | −27.740 | −11.498 | −0.165 | 1.00 | 44.52 | B | C |
| ATOM | 2831 | CD2 | TYR | B | 74 | −26.363 | −11.521 | −0.118 | 1.00 | 42.52 | B | C |
| ATOM | 2832 | C | TYR | B | 74 | −23.990 | −8.348 | −0.685 | 1.00 | 34.57 | B | C |
| ATOM | 2833 | O | TYR | B | 74 | −24.756 | −7.962 | −1.585 | 1.00 | 34.24 | B | O |
| ATOM | 2834 | N | ASP | B | 75 | −23.517 | −7.558 | 0.289 | 1.00 | 32.83 | B | N |
| ATOM | 2835 | CA | ASP | B | 75 | −23.765 | −6.110 | 0.313 | 1.00 | 32.00 | B | C |
| ATOM | 2836 | CB | ASP | B | 75 | −22.966 | −5.400 | 1.423 | 1.00 | 30.80 | B | C |
| ATOM | 2837 | CG | ASP | B | 75 | −23.468 | −5.736 | 2.823 | 1.00 | 29.68 | B | C |
| ATOM | 2838 | OD1 | ASP | B | 75 | −24.558 | −6.343 | 2.956 | 1.00 | 26.51 | B | O |
| ATOM | 2839 | OD2 | ASP | B | 75 | −22.742 | −5.406 | 3.791 | 1.00 | 27.80 | B | O |
| ATOM | 2840 | C | ASP | B | 75 | −23.456 | −5.451 | −1.030 | 1.00 | 32.02 | B | C |
| ATOM | 2841 | O | ASP | B | 75 | −24.321 | −4.795 | −1.602 | 1.00 | 34.17 | B | O |
| ATOM | 2842 | N | TYR | B | 76 | −22.241 | −5.632 | −1.540 | 1.00 | 31.51 | B | N |
| ATOM | 2843 | CA | TYR | B | 76 | −21.849 | −5.008 | −2.805 | 1.00 | 30.68 | B | C |
| ATOM | 2844 | CB | TYR | B | 76 | −20.422 | −5.408 | −3.183 | 1.00 | 31.50 | B | C |
| ATOM | 2845 | CG | TYR | B | 76 | −19.954 | −4.945 | −4.549 | 1.00 | 32.80 | B | C |
| ATOM | 2846 | CD1 | TYR | B | 76 | −20.312 | −5.644 | −5.677 | 1.00 | 33.28 | B | C |
| ATOM | 2847 | CE1 | TYR | B | 76 | −19.893 | −5.247 | −6.928 | 1.00 | 33.91 | B | C |
| ATOM | 2848 | CZ | TYR | B | 76 | −19.094 | −4.147 | −7.077 | 1.00 | 34.08 | B | C |
| ATOM | 2849 | OH | TYR | B | 76 | −18.735 | −3.832 | −8.366 | 1.00 | 36.34 | B | O |
| ATOM | 2850 | CE2 | TYR | B | 76 | −18.696 | −3.416 | −5.974 | 1.00 | 33.35 | B | C |
| ATOM | 2851 | CD2 | TYR | B | 76 | −19.119 | −3.825 | −4.710 | 1.00 | 34.22 | B | C |
| ATOM | 2852 | C | TYR | B | 76 | −22.847 | −5.352 | −3.904 | 1.00 | 31.30 | B | C |
| ATOM | 2853 | O | TYR | B | 76 | −23.356 | −4.456 | −4.550 | 1.00 | 33.09 | B | O |
| ATOM | 2854 | N | TYR | B | 77 | −23.154 | −6.633 | −4.100 | 1.00 | 31.67 | B | N |
| ATOM | 2855 | CA | TYR | B | 77 | −24.106 | −7.030 | −5.143 | 1.00 | 33.09 | B | C |
| ATOM | 2856 | CB | TYR | B | 77 | −24.288 | −8.571 | −5.236 | 1.00 | 34.51 | B | C |
| ATOM | 2857 | CG | TYR | B | 77 | −23.373 | −9.296 | −6.237 | 1.00 | 32.63 | B | C |
| ATOM | 2858 | CD1 | TYR | B | 77 | −22.119 | −9.767 | −5.856 | 1.00 | 32.93 | B | C |
| ATOM | 2859 | CE1 | TYR | B | 77 | −21.279 | −10.412 | −6.756 | 1.00 | 32.14 | B | C |
| ATOM | 2860 | CZ | TYR | B | 77 | −21.695 | −10.624 | −8.049 | 1.00 | 32.06 | B | C |
| ATOM | 2861 | OH | TYR | B | 77 | −20.861 | −11.295 | −8.923 | 1.00 | 32.13 | B | O |
| ATOM | 2862 | CE2 | TYR | B | 77 | −22.936 | −10.168 | −8.450 | 1.00 | 31.38 | B | C |
| ATOM | 2863 | CD2 | TYR | B | 77 | −23.766 | −9.512 | −7.544 | 1.00 | 31.01 | B | C |
| ATOM | 2864 | C | TYR | B | 77 | −25.460 | −6.358 | −4.924 | 1.00 | 33.60 | B | C |
| ATOM | 2865 | O | TYR | B | 77 | −26.025 | −5.791 | −5.864 | 1.00 | 31.95 | B | O |
| ATOM | 2866 | N | LYS | B | 78 | −25.968 | −6.428 | −3.693 | 1.00 | 35.21 | B | N |
| ATOM | 2867 | CA | LYS | B | 78 | −27.226 | −5.743 | −3.305 | 1.00 | 38.16 | B | C |
| ATOM | 2868 | CB | LYS | B | 78 | −27.499 | −5.943 | −1.798 | 1.00 | 41.05 | B | C |
| ATOM | 2869 | CG | LYS | B | 78 | −28.442 | −4.930 | −1.143 | 1.00 | 44.48 | B | C |
| ATOM | 2870 | CD | LYS | B | 78 | −29.548 | −5.574 | −0.305 | 1.00 | 46.27 | B | C |
| ATOM | 2871 | CE | LYS | B | 78 | −30.629 | −6.155 | −1.224 | 1.00 | 49.31 | B | C |
| ATOM | 2872 | NZ | LYS | B | 78 | −31.826 | −6.666 | −0.496 | 1.00 | 50.68 | B | N |
| ATOM | 2873 | C | LYS | B | 78 | −27.214 | −4.242 | −3.666 | 1.00 | 38.50 | B | C |
| ATOM | 2874 | O | LYS | B | 78 | −28.116 | −3.739 | −4.348 | 1.00 | 37.83 | B | O |
| ATOM | 2875 | N | ASN | B | 79 | −26.163 | −3.552 | −3.237 | 1.00 | 36.81 | B | N |
| ATOM | 2876 | CA | ASN | B | 79 | −26.072 | −2.119 | −3.390 | 1.00 | 35.01 | B | C |
| ATOM | 2877 | CB | ASN | B | 79 | −25.037 | −1.568 | −2.428 | 1.00 | 34.32 | B | C |
| ATOM | 2878 | CG | ASN | B | 79 | −25.446 | −1.750 | −0.968 | 1.00 | 34.68 | B | C |
| ATOM | 2879 | OD1 | ASN | B | 79 | −26.640 | −1.847 | −0.631 | 1.00 | 31.67 | B | O |
| ATOM | 2880 | ND2 | ASN | B | 79 | −24.451 | −1.805 | −0.090 | 1.00 | 35.09 | B | N |
| ATOM | 2881 | C | ASN | B | 79 | −25.783 | −1.659 | −4.800 | 1.00 | 36.29 | B | C |
| ATOM | 2882 | O | ASN | B | 79 | −26.446 | −0.757 | −5.290 | 1.00 | 36.69 | B | O |
| ATOM | 2883 | N | LYS | B | 80 | −24.821 | −2.271 | −5.472 | 1.00 | 39.00 | B | N |
| ATOM | 2884 | CA | LYS | B | 80 | −24.394 | −1.768 | −6.782 | 1.00 | 41.10 | B | C |
| ATOM | 2885 | CB | LYS | B | 80 | −22.927 | −2.094 | −7.036 | 1.00 | 45.28 | B | C |
| ATOM | 2886 | CG | LYS | B | 80 | −21.982 | −1.547 | −5.982 | 1.00 | 51.45 | B | C |
| ATOM | 2887 | CD | LYS | B | 80 | −21.444 | −0.164 | −6.329 | 1.00 | 56.78 | B | C |
| ATOM | 2888 | CE | LYS | B | 80 | −20.650 | 0.395 | −5.153 | 1.00 | 60.79 | B | C |
| ATOM | 2889 | NZ | LYS | B | 80 | −21.524 | 0.661 | −3.966 | 1.00 | 62.58 | B | N |
| ATOM | 2890 | C | LYS | B | 80 | −25.202 | −2.277 | −7.965 | 1.00 | 41.13 | B | C |
| ATOM | 2891 | O | LYS | B | 80 | −25.033 | −1.754 | −9.072 | 1.00 | 40.54 | B | O |
| ATOM | 2892 | N | PHE | B | 81 | −26.046 | −3.296 | −7.764 | 1.00 | 40.60 | B | N |
| ATOM | 2893 | CA | PHE | B | 81 | −26.746 | −3.941 | −8.892 | 1.00 | 40.39 | B | C |
| ATOM | 2894 | CB | PHE | B | 81 | −26.047 | −5.257 | −9.274 | 1.00 | 42.38 | B | C |
| ATOM | 2895 | CG | PHE | B | 81 | −24.628 | −5.111 | −9.754 | 1.00 | 43.89 | B | C |
| ATOM | 2896 | CD1 | PHE | B | 81 | −24.300 | −4.227 | −10.770 | 1.00 | 45.90 | B | C |
| ATOM | 2897 | CE1 | PHE | B | 81 | −22.990 | −4.112 | −11.214 | 1.00 | 47.25 | B | C |
| ATOM | 2898 | CZ | PHE | B | 81 | −22.000 | −4.911 | −10.664 | 1.00 | 47.90 | B | C |
| ATOM | 2899 | CE2 | PHE | B | 81 | −22.319 | −5.821 | −9.672 | 1.00 | 46.56 | B | C |
| ATOM | 2900 | CD2 | PHE | B | 81 | −23.626 | −5.921 | −9.226 | 1.00 | 45.90 | B | C |
| ATOM | 2901 | C | PHE | B | 81 | −28.217 | −4.290 | −8.659 | 1.00 | 40.16 | B | C |
| ATOM | 2902 | O | PHE | B | 81 | −28.880 | −4.759 | −9.574 | 1.00 | 39.11 | B | O |
| ATOM | 2903 | N | GLY | B | 82 | −28.720 | −4.127 | −7.443 | 1.00 | 40.78 | B | N |
| ATOM | 2904 | CA | GLY | B | 82 | −30.054 | −4.617 | −7.106 | 1.00 | 42.16 | B | C |
| ATOM | 2905 | C | GLY | B | 82 | −30.193 | −6.135 | −7.065 | 1.00 | 44.48 | B | C |
| ATOM | 2906 | O | GLY | B | 82 | −31.308 | −6.662 | −7.002 | 1.00 | 46.91 | B | O |
| ATOM | 2907 | N | ARG | B | 83 | −29.071 | −6.853 | −7.065 | 1.00 | 44.64 | B | N |
| ATOM | 2908 | CA | ARG | B | 83 | −29.108 | −8.311 | −7.112 | 1.00 | 42.54 | B | C |
| ATOM | 2909 | CB | ARG | B | 83 | −27.970 | −8.851 | −7.980 | 1.00 | 43.50 | B | C |
| ATOM | 2910 | CG | ARG | B | 83 | −27.861 | −10.374 | −7.915 | 1.00 | 44.21 | B | C |
| ATOM | 2911 | CD | ARG | B | 83 | −26.887 | −10.972 | −8.920 | 1.00 | 42.64 | B | C |
| ATOM | 2912 | NE | ARG | B | 83 | −27.330 | −12.309 | −9.284 | 1.00 | 41.71 | B | N |
| ATOM | 2913 | CZ | ARG | B | 83 | −28.056 | −12.605 | −10.359 | 1.00 | 41.59 | B | C |
| ATOM | 2914 | NH1 | ARG | B | 83 | −28.397 | −11.672 | −11.239 | 1.00 | 38.71 | B | N |
| ATOM | 2915 | NH2 | ARG | B | 83 | −28.427 | −13.862 | −10.570 | 1.00 | 44.02 | B | N |
| ATOM | 2916 | C | ARG | B | 83 | −29.078 | −8.955 | −5.722 | 1.00 | 39.03 | B | C |
| ATOM | 2917 | O | ARG | B | 83 | −28.308 | −8.560 | −4.851 | 1.00 | 36.63 | B | O |
| ATOM | 2918 | N | ASN | B | 84 | −29.910 | −9.979 | −5.559 | 1.00 | 37.19 | B | N |
| ATOM | 2919 | CA | ASN | B | 84 | −30.100 | −10.669 | −4.277 | 1.00 | 35.57 | B | C |
| ATOM | 2920 | CB | ASN | B | 84 | −31.602 | −10.752 | −3.969 | 1.00 | 32.60 | B | C |
| ATOM | 2921 | CG | ASN | B | 84 | −31.912 | −11.200 | −2.559 | 1.00 | 30.63 | B | C |
| ATOM | 2922 | OD1 | ASN | B | 84 | −33.073 | −11.419 | −2.241 | 1.00 | 30.00 | B | O |
| ATOM | 2923 | ND2 | ASN | B | 84 | −30.903 | −11.321 | −1.709 | 1.00 | 29.72 | B | N |
| ATOM | 2924 | C | ASN | B | 84 | −29.493 | −12.069 | −4.355 | 1.00 | 35.30 | B | C |
| ATOM | 2925 | O | ASN | B | 84 | −30.018 | −12.925 | −5.050 | 1.00 | 35.02 | B | O |
| ATOM | 2926 | N | SER | B | 85 | −28.379 | −12.275 | −3.648 | 1.00 | 37.34 | B | N |
| ATOM | 2927 | CA | SER | B | 85 | −27.523 | −13.490 | −3.757 | 1.00 | 37.67 | B | C |
| ATOM | 2928 | CB | SER | B | 85 | −28.072 | −14.642 | −2.910 | 1.00 | 37.87 | B | C |
| ATOM | 2929 | OG | SER | B | 85 | −27.019 | −15.470 | −2.452 | 1.00 | 36.06 | B | O |
| ATOM | 2930 | C | SER | B | 85 | −27.271 | −13.918 | −5.209 | 1.00 | 36.47 | B | C |
| ATOM | 2931 | O | SER | B | 85 | −27.404 | −13.093 | −6.117 | 1.00 | 38.17 | B | O |
| ATOM | 2932 | N | ILE | B | 86 | −26.911 | −15.177 | −5.445 | 1.00 | 35.78 | B | N |
| ATOM | 2933 | CA | ILE | B | 86 | −26.422 | −15.556 | −6.788 | 1.00 | 38.90 | B | C |
| ATOM | 2934 | CB | ILE | B | 86 | −25.482 | −16.789 | −6.767 | 1.00 | 40.34 | B | C |
| ATOM | 2935 | CG1 | ILE | B | 86 | −26.280 | −18.073 | −6.741 | 1.00 | 43.61 | B | C |
| ATOM | 2936 | CD1 | ILE | B | 86 | −25.397 | −19.286 | −6.631 | 1.00 | 47.20 | B | C |
| ATOM | 2937 | CG2 | ILE | B | 86 | −24.540 | −16.770 | −5.564 | 1.00 | 40.32 | B | C |
| ATOM | 2938 | C | ILE | B | 86 | −27.543 | −15.744 | −7.829 | 1.00 | 38.42 | B | C |
| ATOM | 2939 | O | ILE | B | 86 | −27.374 | −15.397 | −8.994 | 1.00 | 36.18 | B | O |
| ATOM | 2940 | N | ASP | B | 87 | −28.672 | −16.290 | −7.387 | 1.00 | 41.14 | B | N |
| ATOM | 2941 | CA | ASP | B | 87 | −29.843 | −16.563 | −8.233 | 1.00 | 41.69 | B | C |
| ATOM | 2942 | CB | ASP | B | 87 | −30.586 | −17.806 | −7.714 | 1.00 | 41.60 | B | C |
| ATOM | 2943 | CG | ASP | B | 87 | −31.249 | −17.572 | −6.344 | 1.00 | 41.11 | B | C |
| ATOM | 2944 | OD1 | ASP | B | 87 | −30.790 | −16.683 | −5.603 | 1.00 | 38.26 | B | O |
| ATOM | 2945 | OD2 | ASP | B | 87 | −32.205 | −18.286 | −5.983 | 1.00 | 40.33 | B | O |
| ATOM | 2946 | C | ASP | B | 87 | −30.862 | −15.420 | −8.293 | 1.00 | 43.82 | B | C |
| ATOM | 2947 | O | ASP | B | 87 | −31.952 | −15.624 | −8.833 | 1.00 | 45.59 | B | O |
| ATOM | 2948 | N | GLY | B | 88 | −30.554 | −14.254 | −7.714 | 1.00 | 42.79 | B | N |
| ATOM | 2949 | CA | GLY | B | 88 | −31.467 | −13.107 | −7.774 | 1.00 | 42.83 | B | C |
| ATOM | 2950 | C | GLY | B | 88 | −32.580 | −13.102 | −6.734 | 1.00 | 43.95 | B | C |
| ATOM | 2951 | O | GLY | B | 88 | −33.034 | −12.041 | −6.334 | 1.00 | 41.75 | B | O |
| ATOM | 2952 | N | ASN | B | 89 | −33.061 | −14.284 | −6.346 | 1.00 | 49.48 | B | N |
| ATOM | 2953 | CA | ASN | B | 89 | −33.806 | −14.473 | −5.085 | 1.00 | 52.34 | B | C |
| ATOM | 2954 | CB | ASN | B | 89 | −34.804 | −15.628 | −5.189 | 1.00 | 56.66 | B | C |
| ATOM | 2955 | CG | ASN | B | 89 | −35.843 | −15.406 | −6.287 | 1.00 | 62.28 | B | C |
| ATOM | 2956 | OD1 | ASN | B | 89 | −35.693 | −14.522 | −7.145 | 1.00 | 63.04 | B | O |
| ATOM | 2957 | ND2 | ASN | B | 89 | −36.909 | −16.202 | −6.259 | 1.00 | 63.74 | B | N |
| ATOM | 2958 | C | ASN | B | 89 | −32.735 | −14.793 | −4.086 | 1.00 | 49.08 | B | C |
| ATOM | 2959 | O | ASN | B | 89 | −31.630 | −15.116 | −4.486 | 1.00 | 57.39 | B | O |
| ATOM | 2960 | N | GLY | B | 90 | −33.003 | −14.724 | −2.798 | 1.00 | 44.73 | B | N |
| ATOM | 2961 | CA | GLY | B | 90 | −31.880 | −14.648 | −1.838 | 1.00 | 42.09 | B | C |
| ATOM | 2962 | C | GLY | B | 90 | −31.136 | −15.934 | −1.533 | 1.00 | 40.51 | B | C |
| ATOM | 2963 | O | GLY | B | 90 | −30.983 | −16.274 | −0.367 | 1.00 | 38.47 | B | O |
| ATOM | 2964 | N | PHE | B | 91 | −30.654 | −16.623 | −2.577 | 1.00 | 40.34 | B | N |
| ATOM | 2965 | CA | PHE | B | 91 | −30.044 | −17.961 | −2.471 | 1.00 | 41.32 | B | C |
| ATOM | 2966 | CB | PHE | B | 91 | −29.339 | −18.344 | −3.801 | 1.00 | 45.43 | B | C |
| ATOM | 2967 | CG | PHE | B | 91 | −28.680 | −19.715 | −3.800 | 1.00 | 49.29 | B | C |
| ATOM | 2968 | CD1 | PHE | B | 91 | −29.447 | −20.880 | −3.899 | 1.00 | 48.89 | B | C |
| ATOM | 2969 | CE1 | PHE | B | 91 | −28.837 | −22.121 | −3.886 | 1.00 | 50.44 | B | C |
| ATOM | 2970 | CZ | PHE | B | 91 | −27.450 | −22.219 | −3.792 | 1.00 | 49.76 | B | C |
| ATOM | 2971 | CE2 | PHE | B | 91 | −26.679 | −21.081 | −3.708 | 1.00 | 47.44 | B | C |
| ATOM | 2972 | CD2 | PHE | B | 91 | −27.288 | −19.837 | −3.719 | 1.00 | 49.23 | B | C |
| ATOM | 2973 | C | PHE | B | 91 | −29.071 | −18.048 | −1.308 | 1.00 | 38.84 | B | C |
| ATOM | 2974 | O | PHE | B | 91 | −28.177 | −17.219 | −1.185 | 1.00 | 41.42 | B | O |
| ATOM | 2975 | N | GLN | B | 92 | −29.259 | −19.038 | −0.440 | 1.00 | 38.23 | B | N |
| ATOM | 2976 | CA | GLN | B | 92 | −28.398 | −19.195 | 0.738 | 1.00 | 37.91 | B | C |
| ATOM | 2977 | CB | GLN | B | 92 | −28.943 | −20.290 | 1.660 | 1.00 | 38.21 | B | C |
| ATOM | 2978 | CG | GLN | B | 92 | −28.030 | −20.632 | 2.834 | 1.00 | 41.26 | B | C |
| ATOM | 2979 | CD | GLN | B | 92 | −28.635 | −21.661 | 3.787 | 1.00 | 41.52 | B | C |
| ATOM | 2980 | OE1 | GLN | B | 92 | −29.230 | −22.645 | 3.356 | 1.00 | 44.14 | B | O |
| ATOM | 2981 | NE2 | GLN | B | 92 | −28.453 | −21.451 | 5.079 | 1.00 | 40.35 | B | N |
| ATOM | 2982 | C | GLN | B | 92 | −26.930 | −19.456 | 0.322 | 1.00 | 36.58 | B | C |
| ATOM | 2983 | O | GLN | B | 92 | −26.652 | −20.169 | −0.649 | 1.00 | 38.61 | B | O |
| ATOM | 2984 | N | LEU | B | 93 | −26.003 | −18.857 | 1.055 | 1.00 | 35.02 | B | N |
| ATOM | 2985 | CA | LEU | B | 93 | −24.599 | −18.840 | 0.670 | 1.00 | 35.33 | B | C |
| ATOM | 2986 | CB | LEU | B | 93 | −24.106 | −17.398 | 0.619 | 1.00 | 35.64 | B | C |
| ATOM | 2987 | CG | LEU | B | 93 | −24.765 | −16.482 | −0.435 | 1.00 | 36.84 | B | C |
| ATOM | 2988 | CD1 | LEU | B | 93 | −24.304 | −15.036 | −0.267 | 1.00 | 36.03 | B | C |
| ATOM | 2989 | CD2 | LEU | B | 93 | −24.506 | −16.983 | −1.859 | 1.00 | 37.70 | B | C |
| ATOM | 2990 | C | LEU | B | 93 | −23.745 | −19.658 | 1.632 | 1.00 | 36.40 | B | C |
| ATOM | 2991 | O | LEU | B | 93 | −23.507 | −19.249 | 2.770 | 1.00 | 36.65 | B | O |
| ATOM | 2992 | N | LYS | B | 94 | −23.292 | −20.820 | 1.173 | 1.00 | 37.02 | B | N |
| ATOM | 2993 | CA | LYS | B | 94 | −22.555 | −21.738 | 2.041 | 1.00 | 40.31 | B | C |
| ATOM | 2994 | CB | LYS | B | 94 | −23.009 | −23.211 | 1.813 | 1.00 | 43.72 | B | C |
| ATOM | 2995 | CG | LYS | B | 94 | −24.379 | −23.603 | 2.386 | 1.00 | 44.25 | B | C |
| ATOM | 2996 | CD | LYS | B | 94 | −25.058 | −24.748 | 1.617 | 1.00 | 46.62 | B | C |
| ATOM | 2997 | CE | LYS | B | 94 | −26.527 | −24.981 | 2.021 | 1.00 | 46.67 | B | C |
| ATOM | 2998 | NZ | LYS | B | 94 | −26.707 | −25.189 | 3.493 | 1.00 | 44.46 | B | N |
| ATOM | 2999 | C | LYS | B | 94 | −21.059 | −21.562 | 1.748 | 1.00 | 38.21 | B | C |
| ATOM | 3000 | O | LYS | B | 94 | −20.677 | −21.435 | 0.599 | 1.00 | 36.56 | B | O |
| ATOM | 3001 | N | SER | B | 95 | −20.226 | −21.552 | 2.784 | 1.00 | 35.66 | B | N |
| ATOM | 3002 | CA | SER | B | 95 | −18.801 | −21.364 | 2.615 | 1.00 | 35.30 | B | C |
| ATOM | 3003 | CB | SER | B | 95 | −18.393 | −19.953 | 3.037 | 1.00 | 37.34 | B | C |
| ATOM | 3004 | OG | SER | B | 95 | −19.004 | −18.966 | 2.224 | 1.00 | 40.01 | B | O |
| ATOM | 3005 | C | SER | B | 95 | −18.084 | −22.335 | 3.508 | 1.00 | 35.67 | B | C |
| ATOM | 3006 | O | SER | B | 95 | −18.331 | −22.355 | 4.712 | 1.00 | 35.97 | B | O |
| ATOM | 3007 | N | THR | B | 96 | −17.186 | −23.139 | 2.952 | 1.00 | 34.75 | B | N |
| ATOM | 3008 | CA | THR | B | 96 | −16.447 | −24.087 | 3.788 | 1.00 | 34.33 | B | C |
| ATOM | 3009 | CB | THR | B | 96 | −16.594 | −25.545 | 3.289 | 1.00 | 33.88 | B | C |
| ATOM | 3010 | OG1 | THR | B | 96 | −17.978 | −25.859 | 3.076 | 1.00 | 34.75 | B | O |
| ATOM | 3011 | CG2 | THR | B | 96 | −16.021 | −26.523 | 4.291 | 1.00 | 33.52 | B | C |
| ATOM | 3012 | C | THR | B | 96 | −14.983 | −23.703 | 3.791 | 1.00 | 33.72 | B | C |
| ATOM | 3013 | O | THR | B | 96 | −14.398 | −23.508 | 2.729 | 1.00 | 33.99 | B | O |
| ATOM | 3014 | N | VAL | B | 97 | −14.397 | −23.586 | 4.978 | 1.00 | 33.07 | B | N |
| ATOM | 3015 | CA | VAL | B | 97 | −12.952 | −23.374 | 5.093 | 1.00 | 34.16 | B | C |
| ATOM | 3016 | CB | VAL | B | 97 | −12.592 | −22.282 | 6.128 | 1.00 | 34.28 | B | C |
| ATOM | 3017 | CG1 | VAL | B | 97 | −13.159 | −20.957 | 5.683 | 1.00 | 35.98 | B | C |
| ATOM | 3018 | CG2 | VAL | B | 97 | −13.100 | −22.619 | 7.515 | 1.00 | 35.36 | B | C |
| ATOM | 3019 | C | VAL | B | 97 | −12.224 | −24.672 | 5.432 | 1.00 | 34.05 | B | C |
| ATOM | 3020 | O | VAL | B | 97 | −12.849 | −25.727 | 5.565 | 1.00 | 34.39 | B | O |
| ATOM | 3021 | N | HIS | B | 98 | −10.898 | −24.575 | 5.543 | 1.00 | 33.56 | B | N |
| ATOM | 3022 | CA | HIS | B | 98 | −10.042 | −25.695 | 5.882 | 1.00 | 32.14 | B | C |
| ATOM | 3023 | CB | HIS | B | 98 | −10.257 | −26.060 | 7.328 | 1.00 | 33.55 | B | C |
| ATOM | 3024 | CG | HIS | B | 98 | −10.063 | −24.924 | 8.258 | 1.00 | 34.25 | B | C |
| ATOM | 3025 | ND1 | HIS | B | 98 | −8.841 | −24.315 | 8.436 | 1.00 | 35.12 | B | N |
| ATOM | 3026 | CE1 | HIS | B | 98 | −8.961 | −23.356 | 9.332 | 1.00 | 36.99 | B | C |
| ATOM | 3027 | NE2 | HIS | B | 98 | −10.220 | −23.320 | 9.733 | 1.00 | 37.68 | B | N |
| ATOM | 3028 | CD2 | HIS | B | 98 | −10.927 | −24.299 | 9.084 | 1.00 | 34.92 | B | C |
| ATOM | 3029 | C | HIS | B | 98 | −10.337 | −26.911 | 5.043 | 1.00 | 31.42 | B | C |
| ATOM | 3030 | O | HIS | B | 98 | −10.422 | −28.024 | 5.578 | 1.00 | 30.39 | B | O |
| ATOM | 3031 | N | TYR | B | 99 | −10.524 | −26.701 | 3.738 | 1.00 | 29.84 | B | N |
| ATOM | 3032 | CA | TYR | B | 99 | −10.814 | −27.799 | 2.820 | 1.00 | 28.03 | B | C |
| ATOM | 3033 | CB | TYR | B | 99 | −11.373 | −27.284 | 1.524 | 1.00 | 27.78 | B | C |
| ATOM | 3034 | CG | TYR | B | 99 | −11.713 | −28.379 | 0.558 | 1.00 | 28.82 | B | C |
| ATOM | 3035 | CD1 | TYR | B | 99 | −12.884 | −29.113 | 0.710 | 1.00 | 30.01 | B | C |
| ATOM | 3036 | CE1 | TYR | B | 99 | −13.234 | −30.098 | −0.193 | 1.00 | 29.32 | B | C |
| ATOM | 3037 | CZ | TYR | B | 99 | −12.406 | −30.367 | −1.259 | 1.00 | 28.91 | B | C |
| ATOM | 3038 | OH | TYR | B | 99 | −12.778 | −31.345 | −2.132 | 1.00 | 27.23 | B | O |
| ATOM | 3039 | CE2 | TYR | B | 99 | −11.227 | −29.664 | −1.436 | 1.00 | 28.87 | B | C |
| ATOM | 3040 | CD2 | TYR | B | 99 | −10.888 | −28.671 | −0.526 | 1.00 | 29.25 | B | C |
| ATOM | 3041 | C | TYR | B | 99 | −9.568 | −28.584 | 2.503 | 1.00 | 26.15 | B | C |
| ATOM | 3042 | O | TYR | B | 99 | −8.635 | −28.055 | 1.878 | 1.00 | 24.18 | B | O |
| ATOM | 3043 | N | SER | B | 100 | −9.576 | −29.839 | 2.942 | 1.00 | 24.97 | B | N |
| ATOM | 3044 | CA | SER | B | 100 | −8.515 | −30.805 | 2.656 | 1.00 | 25.57 | B | C |
| ATOM | 3045 | CB | SER | B | 100 | −8.519 | −31.186 | 1.174 | 1.00 | 23.87 | B | C |
| ATOM | 3046 | OG | SER | B | 100 | −7.308 | −31.791 | 0.828 | 1.00 | 22.41 | B | O |
| ATOM | 3047 | C | SER | B | 100 | −7.140 | −30.320 | 3.143 | 1.00 | 26.93 | B | C |
| ATOM | 3048 | O | SER | B | 100 | −7.067 | −29.280 | 3.782 | 1.00 | 28.15 | B | O |
| ATOM | 3049 | N | SER | B | 101 | −6.074 | −31.083 | 2.875 | 1.00 | 27.99 | B | N |
| ATOM | 3050 | CA | SER | B | 101 | −4.750 | −30.814 | 3.463 | 1.00 | 28.45 | B | C |
| ATOM | 3051 | CB | SER | B | 101 | −4.040 | −32.123 | 3.845 | 1.00 | 28.72 | B | C |
| ATOM | 3052 | OG | SER | B | 101 | −2.682 | −32.138 | 3.419 | 1.00 | 29.51 | B | O |
| ATOM | 3053 | C | SER | B | 101 | −3.884 | −29.984 | 2.519 | 1.00 | 29.04 | B | C |
| ATOM | 3054 | O | SER | B | 101 | −3.783 | −30.299 | 1.331 | 1.00 | 27.72 | B | O |
| ATOM | 3055 | N | ARG | B | 102 | −3.289 | −28.919 | 3.069 | 1.00 | 30.50 | B | N |
| ATOM | 3056 | CA | ARG | B | 102 | −2.401 | −27.999 | 2.354 | 1.00 | 30.50 | B | C |
| ATOM | 3057 | CB | ARG | B | 102 | −0.949 | −28.479 | 2.461 | 1.00 | 33.61 | B | C |
| ATOM | 3058 | CG | ARG | B | 102 | −0.281 | −28.133 | 3.800 | 1.00 | 37.70 | B | C |
| ATOM | 3059 | CD | ARG | B | 102 | 1.209 | −28.482 | 3.815 | 1.00 | 40.99 | B | C |
| ATOM | 3060 | NE | ARG | B | 102 | 1.410 | −29.932 | 3.662 | 1.00 | 45.77 | B | N |
| ATOM | 3061 | CZ | ARG | B | 102 | 2.389 | −30.528 | 2.961 | 1.00 | 47.99 | B | C |
| ATOM | 3062 | NH1 | ARG | B | 102 | 3.304 | −29.804 | 2.311 | 1.00 | 50.01 | B | N |
| ATOM | 3063 | NH2 | ARG | B | 102 | 2.460 | −31.870 | 2.914 | 1.00 | 44.90 | B | N |
| ATOM | 3064 | C | ARG | B | 102 | −2.840 | −27.800 | 0.900 | 1.00 | 28.31 | B | C |
| ATOM | 3065 | O | ARG | B | 102 | −2.069 | −27.986 | −0.020 | 1.00 | 27.45 | B | O |
| ATOM | 3066 | N | TYR | B | 103 | −4.098 | −27.396 | 0.740 | 1.00 | 26.02 | B | N |
| ATOM | 3067 | CA | TYR | B | 103 | −4.787 | −27.284 | −0.548 | 1.00 | 23.64 | B | C |
| ATOM | 3068 | CB | TYR | B | 103 | −6.247 | −27.678 | −0.330 | 1.00 | 23.86 | B | C |
| ATOM | 3069 | CG | TYR | B | 103 | −7.039 | −27.730 | −1.575 | 1.00 | 24.13 | B | C |
| ATOM | 3070 | CD1 | TYR | B | 103 | −6.841 | −28.754 | −2.479 | 1.00 | 25.33 | B | C |
| ATOM | 3071 | CE1 | TYR | B | 103 | −7.554 | −28.825 | −3.652 | 1.00 | 25.32 | B | C |
| ATOM | 3072 | CZ | TYR | B | 103 | −8.476 | −27.862 | −3.928 | 1.00 | 26.06 | B | C |
| ATOM | 3073 | OH | TYR | B | 103 | −9.167 | −27.961 | −5.104 | 1.00 | 27.97 | B | O |
| ATOM | 3074 | CE2 | TYR | B | 103 | −8.707 | −26.819 | −3.041 | 1.00 | 24.94 | B | C |
| ATOM | 3075 | CD2 | TYR | B | 103 | −7.979 | −26.763 | −1.869 | 1.00 | 24.27 | B | C |
| ATOM | 3076 | C | TYR | B | 103 | −4.718 | −25.851 | −1.153 | 1.00 | 21.60 | B | C |
| ATOM | 3077 | O | TYR | B | 103 | −5.217 | −24.885 | −0.546 | 1.00 | 19.52 | B | O |
| ATOM | 3078 | N | ASN | B | 104 | −4.110 | −25.743 | −2.349 | 1.00 | 19.76 | B | N |
| ATOM | 3079 | CA | ASN | B | 104 | −3.842 | −24.452 | −3.015 | 1.00 | 18.78 | B | C |
| ATOM | 3080 | CB | ASN | B | 104 | −2.521 | −24.503 | −3.810 | 1.00 | 18.63 | B | C |
| ATOM | 3081 | CG | ASN | B | 104 | −1.267 | −24.521 | −2.930 | 1.00 | 18.22 | B | C |
| ATOM | 3082 | OD1 | ASN | B | 104 | −1.220 | −23.993 | −1.815 | 1.00 | 18.11 | B | O |
| ATOM | 3083 | ND2 | ASN | B | 104 | −0.230 | −25.118 | −3.461 | 1.00 | 17.67 | B | N |
| ATOM | 3084 | C | ASN | B | 104 | −4.969 | −23.980 | −3.962 | 1.00 | 17.89 | B | C |
| ATOM | 3085 | O | ASN | B | 104 | −4.729 | −23.661 | −5.119 | 1.00 | 16.38 | B | O |
| ATOM | 3086 | N | ASN | B | 105 | −6.195 | −23.906 | −3.463 | 1.00 | 17.58 | B | N |
| ATOM | 3087 | CA | ASN | B | 105 | −7.247 | −23.368 | −4.261 | 1.00 | 18.17 | B | C |
| ATOM | 3088 | CB | ASN | B | 105 | −7.549 | −24.310 | −5.395 | 1.00 | 17.95 | B | C |
| ATOM | 3089 | CG | ASN | B | 105 | −7.575 | −23.611 | −6.739 | 1.00 | 18.43 | B | C |
| ATOM | 3090 | OD1 | ASN | B | 105 | −7.983 | −22.445 | −6.890 | 1.00 | 18.76 | B | O |
| ATOM | 3091 | ND2 | ASN | B | 105 | −7.147 | −24.340 | −7.740 | 1.00 | 19.03 | B | N |
| ATOM | 3092 | C | ASN | B | 105 | −8.558 | −23.035 | −3.544 | 1.00 | 20.12 | B | C |
| ATOM | 3093 | O | ASN | B | 105 | −8.807 | −23.426 | −2.391 | 1.00 | 22.03 | B | O |
| ATOM | 3094 | N | ALA | B | 106 | −9.386 | −22.274 | −4.247 | 1.00 | 19.79 | B | N |
| ATOM | 3095 | CA | ALA | B | 106 | −10.718 | −22.010 | −3.819 | 1.00 | 19.99 | B | C |
| ATOM | 3096 | CB | ALA | B | 106 | −10.897 | −20.520 | −3.551 | 1.00 | 20.22 | B | C |
| ATOM | 3097 | C | ALA | B | 106 | −11.540 | −22.440 | −4.990 | 1.00 | 20.21 | B | C |
| ATOM | 3098 | O | ALA | B | 106 | −11.021 | −22.459 | −6.098 | 1.00 | 19.79 | B | O |
| ATOM | 3099 | N | PHE | B | 107 | −12.813 | −22.758 | −4.768 | 1.00 | 21.28 | B | N |
| ATOM | 3100 | CA | PHE | B | 107 | −13.684 | −23.129 | −5.883 | 1.00 | 22.81 | B | C |
| ATOM | 3101 | CB | PHE | B | 107 | −13.343 | −24.553 | −6.408 | 1.00 | 24.23 | B | C |
| ATOM | 3102 | CG | PHE | B | 107 | −13.461 | −25.660 | −5.365 | 1.00 | 25.42 | B | C |
| ATOM | 3103 | CD1 | PHE | B | 107 | −12.387 | −25.980 | −4.532 | 1.00 | 26.00 | B | C |
| ATOM | 3104 | CE1 | PHE | B | 107 | −12.496 | −26.993 | −3.590 | 1.00 | 26.52 | B | C |
| ATOM | 3105 | CZ | PHE | B | 107 | −13.686 | −27.714 | −3.479 | 1.00 | 26.55 | B | C |
| ATOM | 3106 | CE2 | PHE | B | 107 | −14.754 | −27.427 | −4.310 | 1.00 | 25.62 | B | C |
| ATOM | 3107 | CD2 | PHE | B | 107 | −14.640 | −26.408 | −5.247 | 1.00 | 25.85 | B | C |
| ATOM | 3108 | C | PHE | B | 107 | −15.174 | −23.011 | −5.576 | 1.00 | 22.11 | B | C |
| ATOM | 3109 | O | PHE | B | 107 | −15.581 | −22.879 | −4.422 | 1.00 | 21.96 | B | O |
| ATOM | 3110 | N | TRP | B | 108 | −15.962 | −23.035 | −6.644 | 1.00 | 21.93 | B | N |
| ATOM | 3111 | CA | TRP | B | 108 | −17.418 | −23.093 | −6.576 | 1.00 | 22.59 | B | C |
| ATOM | 3112 | CB | TRP | B | 108 | −18.045 | −21.893 | −7.330 | 1.00 | 21.95 | B | C |
| ATOM | 3113 | CG | TRP | B | 108 | −19.427 | −22.087 | −7.888 | 1.00 | 20.54 | B | C |
| ATOM | 3114 | CD1 | TRP | B | 108 | −19.743 | −22.448 | −9.159 | 1.00 | 20.50 | B | C |
| ATOM | 3115 | NE1 | TRP | B | 108 | −21.102 | −22.522 | −9.318 | 1.00 | 20.10 | B | N |
| ATOM | 3116 | CE2 | TRP | B | 108 | −21.701 | −22.176 | −8.143 | 1.00 | 20.03 | B | C |
| ATOM | 3117 | CD2 | TRP | B | 108 | −20.666 | −21.888 | −7.213 | 1.00 | 20.47 | B | C |
| ATOM | 3118 | CE3 | TRP | B | 108 | −21.012 | −21.496 | −5.918 | 1.00 | 20.91 | B | C |
| ATOM | 3119 | CZ3 | TRP | B | 108 | −22.392 | −21.423 | −5.591 | 1.00 | 21.48 | B | C |
| ATOM | 3120 | CH2 | TRP | B | 108 | −23.393 | −21.729 | −6.552 | 1.00 | 20.23 | B | C |
| ATOM | 3121 | CZ2 | TRP | B | 108 | −23.064 | −22.094 | −7.824 | 1.00 | 19.90 | B | C |
| ATOM | 3122 | C | TRP | B | 108 | −17.813 | −24.400 | −7.210 | 1.00 | 24.22 | B | C |
| ATOM | 3123 | O | TRP | B | 108 | −17.449 | −24.649 | −8.357 | 1.00 | 24.63 | B | O |
| ATOM | 3124 | N | ASN | B | 109 | −18.555 | −25.233 | −6.479 | 1.00 | 26.95 | B | N |
| ATOM | 3125 | CA | ASN | B | 109 | −18.965 | −26.547 | −6.990 | 1.00 | 27.50 | B | C |
| ATOM | 3126 | CB | ASN | B | 109 | −18.725 | −27.619 | −5.921 | 1.00 | 27.76 | B | C |
| ATOM | 3127 | CG | ASN | B | 109 | −19.755 | −27.595 | −4.793 | 1.00 | 28.70 | B | C |
| ATOM | 3128 | OD1 | ASN | B | 109 | −20.754 | −26.849 | −4.814 | 1.00 | 27.27 | B | O |
| ATOM | 3129 | ND2 | ASN | B | 109 | −19.510 | −28.443 | −3.785 | 1.00 | 29.61 | B | N |
| ATOM | 3130 | C | ASN | B | 109 | −20.405 | −26.611 | −7.505 | 1.00 | 28.31 | B | C |
| ATOM | 3131 | O | ASN | B | 109 | −21.001 | −27.658 | −7.523 | 1.00 | 28.32 | B | O |
| ATOM | 3132 | N | GLY | B | 110 | −20.971 | −25.490 | −7.919 | 1.00 | 31.03 | B | N |
| ATOM | 3133 | CA | GLY | B | 110 | −22.345 | −25.465 | −8.439 | 1.00 | 32.29 | B | C |
| ATOM | 3134 | C | GLY | B | 110 | −23.355 | −25.216 | −7.333 | 1.00 | 33.16 | B | C |
| ATOM | 3135 | O | GLY | B | 110 | −24.504 | −24.822 | −7.595 | 1.00 | 33.81 | B | O |
| ATOM | 3136 | N | VAL | B | 111 | −22.913 | −25.405 | −6.095 | 1.00 | 33.45 | B | N |
| ATOM | 3137 | CA | VAL | B | 111 | −23.799 | −25.336 | −4.942 | 1.00 | 35.17 | B | C |
| ATOM | 3138 | CB | VAL | B | 111 | −24.201 | −26.785 | −4.535 | 1.00 | 34.30 | B | C |
| ATOM | 3139 | CG1 | VAL | B | 111 | −23.952 | −27.096 | −3.048 | 1.00 | 33.42 | B | C |
| ATOM | 3140 | CG2 | VAL | B | 111 | −25.649 | −27.041 | −4.959 | 1.00 | 34.43 | B | C |
| ATOM | 3141 | C | VAL | B | 111 | −23.244 | −24.503 | −3.758 | 1.00 | 36.29 | B | C |
| ATOM | 3142 | O | VAL | B | 111 | −24.018 | −23.960 | −2.969 | 1.00 | 37.98 | B | O |
| ATOM | 3143 | N | GLN | B | 112 | −21.923 | −24.398 | −3.625 | 1.00 | 35.91 | B | N |
| ATOM | 3144 | CA | GLN | B | 112 | −21.327 | −23.587 | −2.558 | 1.00 | 35.06 | B | C |
| ATOM | 3145 | CB | GLN | B | 112 | −21.404 | −24.355 | −1.234 | 1.00 | 35.50 | B | C |
| ATOM | 3146 | CG | GLN | B | 112 | −20.563 | −25.625 | −1.199 | 1.00 | 34.97 | B | C |
| ATOM | 3147 | CD | GLN | B | 112 | −20.178 | −26.032 | 0.207 | 1.00 | 33.92 | B | C |
| ATOM | 3148 | OE1 | GLN | B | 112 | −20.655 | −27.036 | 0.715 | 1.00 | 34.45 | B | O |
| ATOM | 3149 | NE2 | GLN | B | 112 | −19.318 | −25.250 | 0.843 | 1.00 | 33.47 | B | N |
| ATOM | 3150 | C | GLN | B | 112 | −19.861 | −23.172 | −2.833 | 1.00 | 33.71 | B | C |
| ATOM | 3151 | O | GLN | B | 112 | −19.261 | −23.555 | −3.847 | 1.00 | 34.36 | B | O |
| ATOM | 3152 | N | MET | B | 113 | −19.295 | −22.404 | −1.907 | 1.00 | 30.68 | B | N |
| ATOM | 3153 | CA | MET | B | 113 | −17.933 | −21.911 | −2.017 | 1.00 | 28.51 | B | C |
| ATOM | 3154 | CB | MET | B | 113 | −17.849 | −20.424 | −1.641 | 1.00 | 29.34 | B | C |
| ATOM | 3155 | CG | MET | B | 113 | −18.299 | −19.432 | −2.709 | 1.00 | 29.87 | B | C |
| ATOM | 3156 | SD | MET | B | 113 | −20.078 | −19.409 | −2.981 | 1.00 | 29.78 | B | S |
| ATOM | 3157 | CE | MET | B | 113 | −20.613 | −18.711 | −1.419 | 1.00 | 30.48 | B | C |
| ATOM | 3158 | C | MET | B | 113 | −17.070 | −22.692 | −1.060 | 1.00 | 27.82 | B | C |
| ATOM | 3159 | O | MET | B | 113 | −17.493 | −23.039 | 0.062 | 1.00 | 26.31 | B | O |
| ATOM | 3160 | N | VAL | B | 114 | −15.833 | −22.914 | −1.497 | 1.00 | 27.54 | B | N |
| ATOM | 3161 | CA | VAL | B | 114 | −14.882 | −23.775 | −0.794 | 1.00 | 26.67 | B | C |
| ATOM | 3162 | CB | VAL | B | 114 | −14.799 | −25.172 | −1.472 | 1.00 | 27.48 | B | C |
| ATOM | 3163 | CG1 | VAL | B | 114 | −13.869 | −26.096 | −0.697 | 1.00 | 27.84 | B | C |
| ATOM | 3164 | CG2 | VAL | B | 114 | −16.183 | −25.792 | −1.594 | 1.00 | 27.90 | B | C |
| ATOM | 3165 | C | VAL | B | 114 | −13.510 | −23.112 | −0.845 | 1.00 | 23.88 | B | C |
| ATOM | 3166 | O | VAL | B | 114 | −13.093 | −22.652 | −1.913 | 1.00 | 22.42 | B | O |
| ATOM | 3167 | N | TYR | B | 115 | −12.822 | −23.087 | 0.291 | 1.00 | 21.58 | B | N |
| ATOM | 3168 | CA | TYR | B | 115 | −11.557 | −22.383 | 0.411 | 1.00 | 21.93 | B | C |
| ATOM | 3169 | CB | TYR | B | 115 | −11.718 | −21.092 | 1.229 | 1.00 | 21.40 | B | C |
| ATOM | 3170 | CG | TYR | B | 115 | −12.731 | −20.164 | 0.665 | 1.00 | 21.37 | B | C |
| ATOM | 3171 | CD1 | TYR | B | 115 | −12.421 | −19.308 | −0.358 | 1.00 | 21.96 | B | C |
| ATOM | 3172 | CE1 | TYR | B | 115 | −13.389 | −18.468 | −0.910 | 1.00 | 22.81 | B | C |
| ATOM | 3173 | CZ | TYR | B | 115 | −14.677 | −18.480 | −0.422 | 1.00 | 22.74 | B | C |
| ATOM | 3174 | OH | TYR | B | 115 | −15.665 | −17.644 | −0.940 | 1.00 | 22.96 | B | O |
| ATOM | 3175 | CE2 | TYR | B | 115 | −14.992 | −19.339 | 0.605 | 1.00 | 22.43 | B | C |
| ATOM | 3176 | CD2 | TYR | B | 115 | −14.028 | −20.175 | 1.130 | 1.00 | 22.02 | B | C |
| ATOM | 3177 | C | TYR | B | 115 | −10.500 | −23.244 | 1.092 | 1.00 | 22.23 | B | C |
| ATOM | 3178 | O | TYR | B | 115 | −10.611 | −23.520 | 2.287 | 1.00 | 21.55 | B | O |
| ATOM | 3179 | N | GLY | B | 116 | −9.458 | −23.609 | 0.341 | 1.00 | 21.40 | B | N |
| ATOM | 3180 | CA | GLY | B | 116 | −8.302 | −24.273 | 0.901 | 1.00 | 21.51 | B | C |
| ATOM | 3181 | C | GLY | B | 116 | −7.513 | −23.366 | 1.821 | 1.00 | 21.86 | B | C |
| ATOM | 3182 | O | GLY | B | 116 | −7.773 | −22.181 | 1.900 | 1.00 | 22.60 | B | O |
| ATOM | 3183 | N | ASP | B | 117 | −6.549 | −23.936 | 2.523 | 1.00 | 22.59 | B | N |
| ATOM | 3184 | CA | ASP | B | 117 | −5.689 | −23.176 | 3.412 | 1.00 | 23.66 | B | C |
| ATOM | 3185 | CB | ASP | B | 117 | −5.446 | −23.933 | 4.723 | 1.00 | 24.06 | B | C |
| ATOM | 3186 | CG | ASP | B | 117 | −6.583 | −23.800 | 5.710 | 1.00 | 24.67 | B | C |
| ATOM | 3187 | OD1 | ASP | B | 117 | −7.346 | −22.803 | 5.728 | 1.00 | 25.59 | B | O |
| ATOM | 3188 | OD2 | ASP | B | 117 | −6.695 | −24.723 | 6.502 | 1.00 | 25.80 | B | O |
| ATOM | 3189 | C | ASP | B | 117 | −4.346 | −22.926 | 2.766 | 1.00 | 23.69 | B | C |
| ATOM | 3190 | O | ASP | B | 117 | −3.480 | −22.273 | 3.347 | 1.00 | 23.57 | B | O |
| ATOM | 3191 | N | GLY | B | 118 | −4.152 | −23.465 | 1.575 | 1.00 | 24.06 | B | N |
| ATOM | 3192 | CA | GLY | B | 118 | −2.868 | −23.321 | 0.912 | 1.00 | 24.13 | B | C |
| ATOM | 3193 | C | GLY | B | 118 | −1.737 | −24.045 | 1.622 | 1.00 | 22.97 | B | C |
| ATOM | 3194 | O | GLY | B | 118 | −1.779 | −24.292 | 2.834 | 1.00 | 22.31 | B | O |
| ATOM | 3195 | N | ASP | B | 119 | −0.709 | −24.364 | 0.847 | 1.00 | 22.43 | B | N |
| ATOM | 3196 | CA | ASP | B | 119 | 0.478 | −25.039 | 1.368 | 1.00 | 21.83 | B | C |
| ATOM | 3197 | CB | ASP | B | 119 | 1.323 | −25.608 | 0.197 | 1.00 | 20.08 | B | C |
| ATOM | 3198 | CG | ASP | B | 119 | 2.036 | −24.543 | −0.596 | 1.00 | 19.22 | B | C |
| ATOM | 3199 | OD1 | ASP | B | 119 | 2.324 | −23.446 | −0.078 | 1.00 | 19.73 | B | O |
| ATOM | 3200 | OD2 | ASP | B | 119 | 2.347 | −24.810 | −1.752 | 1.00 | 17.52 | B | O |
| ATOM | 3201 | C | ASP | B | 119 | 1.321 | −24.187 | 2.353 | 1.00 | 21.78 | B | C |
| ATOM | 3202 | O | ASP | B | 119 | 2.321 | −24.651 | 2.875 | 1.00 | 21.12 | B | O |
| ATOM | 3203 | N | GLY | B | 120 | 0.916 | −22.943 | 2.587 | 1.00 | 23.30 | B | N |
| ATOM | 3204 | CA | GLY | B | 120 | 1.636 | −22.047 | 3.486 | 1.00 | 23.87 | B | C |
| ATOM | 3205 | C | GLY | B | 120 | 2.932 | −21.462 | 2.957 | 1.00 | 24.49 | B | C |
| ATOM | 3206 | O | GLY | B | 120 | 3.629 | −20.779 | 3.676 | 1.00 | 24.47 | B | O |
| ATOM | 3207 | N | VAL | B | 121 | 3.284 | −21.718 | 1.711 | 1.00 | 26.95 | B | N |
| ATOM | 3208 | CA | VAL | B | 121 | 4.523 | −21.143 | 1.163 | 1.00 | 29.85 | B | C |
| ATOM | 3209 | CB | VAL | B | 121 | 5.613 | −22.221 | 0.856 | 1.00 | 29.49 | B | C |
| ATOM | 3210 | CG1 | VAL | B | 121 | 6.777 | −21.618 | 0.072 | 1.00 | 28.64 | B | C |
| ATOM | 3211 | CG2 | VAL | B | 121 | 6.142 | −22.843 | 2.140 | 1.00 | 28.93 | B | C |
| ATOM | 3212 | C | VAL | B | 121 | 4.211 | −20.346 | −0.094 | 1.00 | 31.76 | B | C |
| ATOM | 3213 | O | VAL | B | 121 | 4.770 | −19.262 | −0.314 | 1.00 | 36.09 | B | O |
| ATOM | 3214 | N | THR | B | 122 | 3.333 | −20.892 | −0.924 | 1.00 | 30.83 | B | N |
| ATOM | 3215 | CA | THR | B | 122 | 2.924 | −20.223 | −2.139 | 1.00 | 29.94 | B | C |
| ATOM | 3216 | CB | THR | B | 122 | 2.957 | −21.220 | −3.312 | 1.00 | 32.47 | B | C |
| ATOM | 3217 | OG1 | THR | B | 122 | 2.621 | −22.519 | −2.826 | 1.00 | 33.51 | B | O |
| ATOM | 3218 | CG2 | THR | B | 122 | 4.399 | −21.336 | −3.879 | 1.00 | 35.40 | B | C |
| ATOM | 3219 | C | THR | B | 122 | 1.562 | −19.555 | −1.934 | 1.00 | 26.62 | B | C |
| ATOM | 3220 | O | THR | B | 122 | 1.221 | −18.626 | −2.642 | 1.00 | 26.32 | B | O |
| ATOM | 3221 | N | PHE | B | 123 | 0.790 | −20.010 | −0.955 | 1.00 | 23.60 | B | N |
| ATOM | 3222 | CA | PHE | B | 123 | −0.483 | −19.367 | −0.627 | 1.00 | 21.85 | B | C |
| ATOM | 3223 | CB | PHE | B | 123 | −1.700 | −20.064 | −1.261 | 1.00 | 21.42 | B | C |
| ATOM | 3224 | CG | PHE | B | 123 | −1.758 | −19.968 | −2.752 | 1.00 | 20.41 | B | C |
| ATOM | 3225 | CD2 | PHE | B | 123 | −2.454 | −18.948 | −3.362 | 1.00 | 20.18 | B | C |
| ATOM | 3226 | CE2 | PHE | B | 123 | −2.516 | −18.853 | −4.741 | 1.00 | 20.20 | B | C |
| ATOM | 3227 | CZ | PHE | B | 123 | −1.865 | −19.781 | −5.527 | 1.00 | 20.04 | B | C |
| ATOM | 3228 | CE1 | PHE | B | 123 | −1.156 | −20.809 | −4.928 | 1.00 | 20.65 | B | C |
| ATOM | 3229 | CD1 | PHE | B | 123 | −1.120 | −20.906 | −3.544 | 1.00 | 20.63 | B | C |
| ATOM | 3230 | C | PHE | B | 123 | −0.669 | −19.445 | 0.854 | 1.00 | 21.39 | B | C |
| ATOM | 3231 | O | PHE | B | 123 | −0.128 | −20.345 | 1.499 | 1.00 | 23.40 | B | O |
| ATOM | 3232 | N | ILE | B | 124 | −1.434 | −18.499 | 1.383 | 1.00 | 20.17 | B | N |
| ATOM | 3233 | CA | ILE | B | 124 | −1.961 | −18.572 | 2.736 | 1.00 | 18.39 | B | C |
| ATOM | 3234 | CB | ILE | B | 124 | −1.640 | −17.310 | 3.541 | 1.00 | 17.54 | B | C |
| ATOM | 3235 | CG1 | ILE | B | 124 | −2.396 | −16.082 | 3.033 | 1.00 | 16.90 | B | C |
| ATOM | 3236 | CD1 | ILE | B | 124 | −2.386 | −14.964 | 4.038 | 1.00 | 16.17 | B | C |
| ATOM | 3237 | CG2 | ILE | B | 124 | −0.147 | −17.048 | 3.498 | 1.00 | 17.65 | B | C |
| ATOM | 3238 | C | ILE | B | 124 | −3.444 | −18.826 | 2.549 | 1.00 | 18.58 | B | C |
| ATOM | 3239 | O | ILE | B | 124 | −3.891 | −18.998 | 1.407 | 1.00 | 18.36 | B | O |
| ATOM | 3240 | N | PRO | B | 125 | −4.203 | −18.918 | 3.649 | 1.00 | 18.87 | B | N |
| ATOM | 3241 | CA | PRO | B | 125 | −5.577 | −19.377 | 3.443 | 1.00 | 19.02 | B | C |
| ATOM | 3242 | CB | PRO | B | 125 | −6.169 | −19.403 | 4.854 | 1.00 | 19.23 | B | C |
| ATOM | 3243 | CG | PRO | B | 125 | −4.980 | −19.587 | 5.754 | 1.00 | 19.46 | B | C |
| ATOM | 3244 | CD | PRO | B | 125 | −3.825 | −18.905 | 5.077 | 1.00 | 19.29 | B | C |
| ATOM | 3245 | C | PRO | B | 125 | −6.356 | −18.467 | 2.510 | 1.00 | 19.07 | B | C |
| ATOM | 3246 | O | PRO | B | 125 | −6.278 | −17.247 | 2.613 | 1.00 | 18.93 | B | O |
| ATOM | 3247 | N | PHE | B | 126 | −7.105 | −19.080 | 1.604 | 1.00 | 18.81 | B | N |
| ATOM | 3248 | CA | PHE | B | 126 | −7.614 | −18.381 | 0.437 | 1.00 | 18.56 | B | C |
| ATOM | 3249 | CB | PHE | B | 126 | −8.131 | −19.385 | −0.604 | 1.00 | 18.49 | B | C |
| ATOM | 3250 | CG | PHE | B | 126 | −7.064 | −19.911 | −1.508 | 1.00 | 18.66 | B | C |
| ATOM | 3251 | CD1 | PHE | B | 126 | −6.020 | −20.664 | −1.006 | 1.00 | 19.04 | B | C |
| ATOM | 3252 | CE1 | PHE | B | 126 | −5.020 | −21.148 | −1.831 | 1.00 | 18.97 | B | C |
| ATOM | 3253 | CZ | PHE | B | 126 | −5.055 | −20.874 | −3.176 | 1.00 | 19.04 | B | C |
| ATOM | 3254 | CE2 | PHE | B | 126 | −6.100 | −20.132 | −3.692 | 1.00 | 18.90 | B | C |
| ATOM | 3255 | CD2 | PHE | B | 126 | −7.098 | −19.653 | −2.860 | 1.00 | 18.72 | B | C |
| ATOM | 3256 | C | PHE | B | 126 | −8.703 | −17.362 | 0.755 | 1.00 | 18.62 | B | C |
| ATOM | 3257 | O | PHE | B | 126 | −9.003 | −16.516 | −0.081 | 1.00 | 19.38 | B | O |
| ATOM | 3258 | N | SER | B | 127 | −9.292 | −17.422 | 1.945 | 1.00 | 17.97 | B | N |
| ATOM | 3259 | CA | SER | B | 127 | −10.348 | −16.474 | 2.291 | 1.00 | 17.25 | B | C |
| ATOM | 3260 | CB | SER | B | 127 | −11.203 | −17.006 | 3.427 | 1.00 | 17.21 | B | C |
| ATOM | 3261 | OG | SER | B | 127 | −10.384 | −17.220 | 4.554 | 1.00 | 17.32 | B | O |
| ATOM | 3262 | C | SER | B | 127 | −9.808 | −15.133 | 2.712 | 1.00 | 16.62 | B | C |
| ATOM | 3263 | O | SER | B | 127 | −10.606 | −14.248 | 2.963 | 1.00 | 16.39 | B | O |
| ATOM | 3264 | N | ALA | B | 128 | −8.481 | −14.984 | 2.782 | 1.00 | 16.07 | B | N |
| ATOM | 3265 | CA | ALA | B | 128 | −7.847 | −13.752 | 3.257 | 1.00 | 15.85 | B | C |
| ATOM | 3266 | CB | ALA | B | 128 | −6.407 | −14.027 | 3.600 | 1.00 | 15.64 | B | C |
| ATOM | 3267 | C | ALA | B | 128 | −7.920 | −12.530 | 2.315 | 1.00 | 16.85 | B | C |
| ATOM | 3268 | O | ALA | B | 128 | −7.759 | −11.373 | 2.775 | 1.00 | 17.34 | B | O |
| ATOM | 3269 | N | ASP | B | 129 | −8.144 | −12.759 | 1.019 | 1.00 | 17.61 | B | N |
| ATOM | 3270 | CA | ASP | B | 129 | −8.243 | −11.666 | 0.041 | 1.00 | 17.88 | B | C |
| ATOM | 3271 | CB | ASP | B | 129 | −7.283 | −11.861 | −1.165 | 1.00 | 18.05 | B | C |
| ATOM | 3272 | CG | ASP | B | 129 | −7.173 | −10.596 | −2.055 | 1.00 | 19.36 | B | C |
| ATOM | 3273 | OD1 | ASP | B | 129 | −8.206 | −9.927 | −2.291 | 1.00 | 19.57 | B | O |
| ATOM | 3274 | OD2 | ASP | B | 129 | −6.063 | −10.247 | −2.541 | 1.00 | 20.33 | B | O |
| ATOM | 3275 | C | ASP | B | 129 | −9.681 | −11.618 | −0.426 | 1.00 | 17.31 | B | C |
| ATOM | 3276 | O | ASP | B | 129 | −10.152 | −12.569 | −1.029 | 1.00 | 17.85 | B | O |
| ATOM | 3277 | N | PRO | B | 130 | −10.371 | −10.493 | −0.196 | 1.00 | 17.22 | B | N |
| ATOM | 3278 | CA | PRO | B | 130 | −11.786 | −10.369 | −0.601 | 1.00 | 17.02 | B | C |
| ATOM | 3279 | CB | PRO | B | 130 | −12.140 | −8.911 | −0.243 | 1.00 | 16.73 | B | C |
| ATOM | 3280 | CG | PRO | B | 130 | −11.099 | −8.456 | 0.723 | 1.00 | 16.71 | B | C |
| ATOM | 3281 | CD | PRO | B | 130 | −9.855 | −9.235 | 0.392 | 1.00 | 17.19 | B | C |
| ATOM | 3282 | C | PRO | B | 130 | −12.074 | −10.631 | −2.085 | 1.00 | 16.62 | B | C |
| ATOM | 3283 | O | PRO | B | 130 | −13.211 | −10.958 | −2.436 | 1.00 | 15.51 | B | O |
| ATOM | 3284 | N | ASP | B | 131 | −11.072 | −10.485 | −2.948 | 1.00 | 16.98 | B | N |
| ATOM | 3285 | CA | ASP | B | 131 | −11.310 | −10.688 | −4.384 | 1.00 | 18.29 | B | C |
| ATOM | 3286 | CB | ASP | B | 131 | −10.235 | −9.999 | −5.283 | 1.00 | 19.03 | B | C |
| ATOM | 3287 | CG | ASP | B | 131 | −8.835 | −10.602 | −5.142 | 1.00 | 19.39 | B | C |
| ATOM | 3288 | OD1 | ASP | B | 131 | −8.691 | −11.825 | −5.259 | 1.00 | 19.96 | B | O |
| ATOM | 3289 | OD2 | ASP | B | 131 | −7.859 | −9.851 | −4.923 | 1.00 | 20.07 | B | O |
| ATOM | 3290 | C | ASP | B | 131 | −11.495 | −12.177 | −4.702 | 1.00 | 19.01 | B | C |
| ATOM | 3291 | O | ASP | B | 131 | −12.097 | −12.542 | −5.721 | 1.00 | 17.48 | B | O |
| ATOM | 3292 | N | VAL | B | 132 | −10.995 | −13.028 | −3.804 | 1.00 | 19.89 | B | N |
| ATOM | 3293 | CA | VAL | B | 132 | −11.221 | −14.456 | −3.922 | 1.00 | 21.16 | B | C |
| ATOM | 3294 | CB | VAL | B | 132 | −10.333 | −15.232 | −2.948 | 1.00 | 22.10 | B | C |
| ATOM | 3295 | CG1 | VAL | B | 132 | −10.779 | −16.695 | −2.865 | 1.00 | 22.23 | B | C |
| ATOM | 3296 | CG2 | VAL | B | 132 | −8.867 | −15.110 | −3.357 | 1.00 | 22.87 | B | C |
| ATOM | 3297 | C | VAL | B | 132 | −12.683 | −14.825 | −3.651 | 1.00 | 21.46 | B | C |
| ATOM | 3298 | O | VAL | B | 132 | −13.300 | −15.590 | −4.402 | 1.00 | 20.31 | B | O |
| ATOM | 3299 | N | ILE | B | 133 | −13.220 | −14.282 | −2.562 | 1.00 | 21.93 | B | N |
| ATOM | 3300 | CA | ILE | B | 133 | −14.609 | −14.519 | −2.215 | 1.00 | 21.56 | B | C |
| ATOM | 3301 | CB | ILE | B | 133 | −15.025 | −13.773 | −0.956 | 1.00 | 21.14 | B | C |
| ATOM | 3302 | CG1 | ILE | B | 133 | −14.046 | −14.023 | 0.199 | 1.00 | 22.31 | B | C |
| ATOM | 3303 | CD1 | ILE | B | 133 | −13.855 | −15.454 | 0.620 | 1.00 | 22.87 | B | C |
| ATOM | 3304 | CG2 | ILE | B | 133 | −16.455 | −14.105 | −0.599 | 1.00 | 20.64 | B | C |
| ATOM | 3305 | C | ILE | B | 133 | −15.465 | −13.987 | −3.346 | 1.00 | 22.51 | B | C |
| ATOM | 3306 | O | ILE | B | 133 | −16.421 | −14.642 | −3.763 | 1.00 | 24.23 | B | O |
| ATOM | 3307 | N | GLY | B | 134 | −15.108 | −12.798 | −3.844 | 1.00 | 22.08 | B | N |
| ATOM | 3308 | CA | GLY | B | 134 | −15.828 | −12.177 | −4.929 | 1.00 | 21.41 | B | C |
| ATOM | 3309 | C | GLY | B | 134 | −15.719 | −13.033 | −6.162 | 1.00 | 22.33 | B | C |
| ATOM | 3310 | O | GLY | B | 134 | −16.690 | −13.223 | −6.891 | 1.00 | 23.18 | B | O |
| ATOM | 3311 | N | HIS | B | 135 | −14.528 | −13.569 | −6.390 | 1.00 | 23.11 | B | N |
| ATOM | 3312 | CA | HIS | B | 135 | −14.280 | −14.385 | −7.573 | 1.00 | 23.08 | B | C |
| ATOM | 3313 | CB | HIS | B | 135 | −12.829 | −14.804 | −7.601 | 1.00 | 21.45 | B | C |
| ATOM | 3314 | CG | HIS | B | 135 | −12.442 | −15.596 | −8.804 | 1.00 | 20.35 | B | C |
| ATOM | 3315 | ND1 | HIS | B | 135 | −12.056 | −15.007 | −9.982 | 1.00 | 19.67 | B | N |
| ATOM | 3316 | CE1 | HIS | B | 135 | −11.717 | −15.942 | −10.847 | 1.00 | 19.76 | B | C |
| ATOM | 3317 | NE2 | HIS | B | 135 | −11.876 | −17.120 | −10.274 | 1.00 | 19.91 | B | N |
| ATOM | 3318 | CD2 | HIS | B | 135 | −12.317 | −16.930 | −8.991 | 1.00 | 20.18 | B | C |
| ATOM | 3319 | C | HIS | B | 135 | −15.164 | −15.628 | −7.593 | 1.00 | 25.10 | B | C |
| ATOM | 3320 | O | HIS | B | 135 | −15.726 | −15.994 | −8.651 | 1.00 | 27.12 | B | O |
| ATOM | 3321 | N | GLU | B | 136 | −15.295 | −16.272 | −6.436 | 1.00 | 25.03 | B | N |
| ATOM | 3322 | CA | GLU | B | 136 | −16.004 | −17.549 | −6.379 | 1.00 | 25.06 | B | C |
| ATOM | 3323 | CB | GLU | B | 136 | −15.518 | −18.377 | −5.191 | 1.00 | 24.35 | B | C |
| ATOM | 3324 | CG | GLU | B | 136 | −14.098 | −18.882 | −5.369 | 1.00 | 24.50 | B | C |
| ATOM | 3325 | CD | GLU | B | 136 | −13.846 | −19.503 | −6.744 | 1.00 | 25.78 | B | C |
| ATOM | 3326 | OE1 | GLU | B | 136 | −12.713 | −19.404 | −7.229 | 1.00 | 29.10 | B | O |
| ATOM | 3327 | OE2 | GLU | B | 136 | −14.753 | −20.096 | −7.371 | 1.00 | 25.51 | B | O |
| ATOM | 3328 | C | GLU | B | 136 | −17.519 | −17.378 | −6.375 | 1.00 | 25.55 | B | C |
| ATOM | 3329 | O | GLU | B | 136 | −18.227 | −18.100 | −7.086 | 1.00 | 23.74 | B | O |
| ATOM | 3330 | N | LEU | B | 137 | −17.993 | −16.404 | −5.594 | 1.00 | 26.39 | B | N |
| ATOM | 3331 | CA | LEU | B | 137 | −19.406 | −16.077 | −5.519 | 1.00 | 26.59 | B | C |
| ATOM | 3332 | CB | LEU | B | 137 | −19.572 | −14.815 | −4.694 | 1.00 | 27.82 | B | C |
| ATOM | 3333 | CG | LEU | B | 137 | −20.822 | −14.488 | −3.869 | 1.00 | 29.11 | B | C |
| ATOM | 3334 | CD1 | LEU | B | 137 | −22.031 | −14.318 | −4.757 | 1.00 | 30.30 | B | C |
| ATOM | 3335 | CD2 | LEU | B | 137 | −21.094 | −15.501 | −2.771 | 1.00 | 30.20 | B | C |
| ATOM | 3336 | C | LEU | B | 137 | −19.893 | −15.841 | −6.930 | 1.00 | 28.03 | B | C |
| ATOM | 3337 | O | LEU | B | 137 | −20.952 | −16.353 | −7.334 | 1.00 | 29.88 | B | O |
| ATOM | 3338 | N | THR | B | 138 | −19.098 | −15.068 | −7.677 | 1.00 | 28.04 | B | N |
| ATOM | 3339 | CA | THR | B | 138 | −19.400 | −14.703 | −9.054 | 1.00 | 27.52 | B | C |
| ATOM | 3340 | CB | THR | B | 138 | −18.403 | −13.654 | −9.578 | 1.00 | 27.58 | B | C |
| ATOM | 3341 | OG1 | THR | B | 138 | −18.419 | −12.512 | −8.720 | 1.00 | 25.48 | B | O |
| ATOM | 3342 | CG2 | THR | B | 138 | −18.744 | −13.228 | −11.013 | 1.00 | 27.35 | B | C |
| ATOM | 3343 | C | THR | B | 138 | −19.371 | −15.928 | −9.967 | 1.00 | 28.01 | B | C |
| ATOM | 3344 | O | THR | B | 138 | −20.163 | −16.016 | −10.896 | 1.00 | 29.23 | B | O |
| ATOM | 3345 | N | HIS | B | 139 | −18.472 | −16.881 | −9.715 | 1.00 | 28.67 | B | N |
| ATOM | 3346 | CA | HIS | B | 139 | −18.529 | −18.184 | −10.421 | 1.00 | 27.39 | B | C |
| ATOM | 3347 | CB | HIS | B | 139 | −17.532 | −19.196 | −9.824 | 1.00 | 26.16 | B | C |
| ATOM | 3348 | CG | HIS | B | 139 | −16.138 | −19.145 | −10.411 | 1.00 | 25.53 | B | C |
| ATOM | 3349 | ND1 | HIS | B | 139 | −15.900 | −19.058 | −11.764 | 1.00 | 25.15 | B | N |
| ATOM | 3350 | CE1 | HIS | B | 139 | −14.595 | −19.065 | −11.988 | 1.00 | 24.31 | B | C |
| ATOM | 3351 | NE2 | HIS | B | 139 | −13.974 | −19.181 | −10.830 | 1.00 | 24.44 | B | N |
| ATOM | 3352 | CD2 | HIS | B | 139 | −14.915 | −19.249 | −9.829 | 1.00 | 24.67 | B | C |
| ATOM | 3353 | C | HIS | B | 139 | −19.987 | −18.721 | −10.357 | 1.00 | 28.40 | B | C |
| ATOM | 3354 | O | HIS | B | 139 | −20.511 | −19.275 | −11.337 | 1.00 | 27.07 | B | O |
| ATOM | 3355 | N | GLY | B | 140 | −20.640 | −18.506 | −9.209 | 1.00 | 29.45 | B | N |
| ATOM | 3356 | CA | GLY | B | 140 | −22.074 | −18.781 | −9.033 | 1.00 | 30.38 | B | C |
| ATOM | 3357 | C | GLY | B | 140 | −23.047 | −17.934 | −9.872 | 1.00 | 31.74 | B | C |
| ATOM | 3358 | O | GLY | B | 140 | −23.870 | −18.491 | −10.622 | 1.00 | 32.56 | B | O |
| ATOM | 3359 | N | VAL | B | 141 | −22.982 | −16.605 | −9.742 | 1.00 | 30.73 | B | N |
| ATOM | 3360 | CA | VAL | B | 141 | −23.765 | −15.712 | −10.609 | 1.00 | 30.84 | B | C |
| ATOM | 3361 | CB | VAL | B | 141 | −23.302 | −14.229 | −10.488 | 1.00 | 30.98 | B | C |
| ATOM | 3362 | CG1 | VAL | B | 141 | −24.079 | −13.307 | −11.417 | 1.00 | 30.38 | B | C |
| ATOM | 3363 | CG2 | VAL | B | 141 | −23.418 | −13.731 | −9.059 | 1.00 | 33.19 | B | C |
| ATOM | 3364 | C | VAL | B | 141 | −23.653 | −16.184 | −12.087 | 1.00 | 32.64 | B | C |
| ATOM | 3365 | O | VAL | B | 141 | −24.680 | −16.395 | −12.764 | 1.00 | 34.17 | B | O |
| ATOM | 3366 | N | THR | B | 142 | −22.425 | −16.371 | −12.584 | 1.00 | 30.57 | B | N |
| ATOM | 3367 | CA | THR | B | 142 | −22.238 | −16.711 | −13.978 | 1.00 | 30.33 | B | C |
| ATOM | 3368 | CB | THR | B | 142 | −20.747 | −16.814 | −14.342 | 1.00 | 28.78 | B | C |
| ATOM | 3369 | OG1 | THR | B | 142 | −20.107 | −15.548 | −14.169 | 1.00 | 28.27 | B | O |
| ATOM | 3370 | CG2 | THR | B | 142 | −20.562 | −17.241 | −15.778 | 1.00 | 28.27 | B | C |
| ATOM | 3371 | C | THR | B | 142 | −23.018 | −18.012 | −14.321 | 1.00 | 34.44 | B | C |
| ATOM | 3372 | O | THR | B | 142 | −23.756 | −18.043 | −15.311 | 1.00 | 31.77 | B | O |
| ATOM | 3373 | N | GLU | B | 143 | −22.891 | −19.058 | −13.496 | 1.00 | 39.28 | B | N |
| ATOM | 3374 | CA | GLU | B | 143 | −23.609 | −20.331 | −13.746 | 1.00 | 44.10 | B | C |
| ATOM | 3375 | CB | GLU | B | 143 | −23.199 | −21.447 | −12.749 | 1.00 | 46.26 | B | C |
| ATOM | 3376 | CG | GLU | B | 143 | −23.758 | −22.829 | −13.098 | 1.00 | 46.77 | B | C |
| ATOM | 3377 | CD | GLU | B | 143 | −23.520 | −23.911 | −12.036 | 1.00 | 50.51 | B | C |
| ATOM | 3378 | OE1 | GLU | B | 143 | −24.476 | −24.652 | −11.712 | 1.00 | 52.85 | B | O |
| ATOM | 3379 | OE2 | GLU | B | 143 | −22.388 | −24.056 | −11.531 | 1.00 | 51.61 | B | O |
| ATOM | 3380 | C | GLU | B | 143 | −25.141 | −20.147 | −13.737 | 1.00 | 43.41 | B | C |
| ATOM | 3381 | O | GLU | B | 143 | −25.837 | −20.722 | −14.579 | 1.00 | 42.45 | B | O |
| ATOM | 3382 | N | HIS | B | 144 | −25.657 | −19.350 | −12.803 | 1.00 | 41.46 | B | N |
| ATOM | 3383 | CA | HIS | B | 144 | −27.095 | −19.050 | −12.768 | 1.00 | 42.89 | B | C |
| ATOM | 3384 | CB | HIS | B | 144 | −27.500 | −18.615 | −11.363 | 1.00 | 43.93 | B | C |
| ATOM | 3385 | CG | HIS | B | 144 | −27.711 | −19.748 | −10.415 | 1.00 | 48.17 | B | C |
| ATOM | 3386 | ND1 | HIS | B | 144 | −26.683 | −20.562 | −9.985 | 1.00 | 53.25 | B | N |
| ATOM | 3387 | CE1 | HIS | B | 144 | −27.156 | −21.457 | −9.134 | 1.00 | 53.12 | B | C |
| ATOM | 3388 | NE2 | HIS | B | 144 | −28.453 | −21.245 | −8.991 | 1.00 | 52.99 | B | N |
| ATOM | 3389 | CD2 | HIS | B | 144 | −28.827 | −20.187 | −9.787 | 1.00 | 49.42 | B | C |
| ATOM | 3390 | C | HIS | B | 144 | −27.596 | −17.985 | −13.790 | 1.00 | 41.27 | B | C |
| ATOM | 3391 | O | HIS | B | 144 | −28.773 | −17.664 | −13.803 | 1.00 | 41.71 | B | O |
| ATOM | 3392 | N | THR | B | 145 | −26.729 | −17.451 | −14.646 | 1.00 | 39.46 | B | N |
| ATOM | 3393 | CA | THR | B | 145 | −27.124 | −16.412 | −15.614 | 1.00 | 37.58 | B | C |
| ATOM | 3394 | CB | THR | B | 145 | −26.559 | −15.013 | −15.211 | 1.00 | 39.34 | B | C |
| ATOM | 3395 | OG1 | THR | B | 145 | −25.127 | −15.071 | −15.027 | 1.00 | 36.83 | B | O |
| ATOM | 3396 | CG2 | THR | B | 145 | −27.213 | −14.517 | −13.918 | 1.00 | 39.08 | B | C |
| ATOM | 3397 | C | THR | B | 145 | −26.685 | −16.769 | −17.042 | 1.00 | 34.70 | B | C |
| ATOM | 3398 | O | THR | B | 145 | −27.380 | −17.459 | −17.750 | 1.00 | 36.43 | B | O |
| ATOM | 3399 | N | ALA | B | 146 | −25.523 | −16.305 | −17.455 | 1.00 | 33.64 | B | N |
| ATOM | 3400 | CA | ALA | B | 146 | −25.006 | −16.605 | −18.769 | 1.00 | 33.70 | B | C |
| ATOM | 3401 | CB | ALA | B | 146 | −23.739 | −15.803 | −19.000 | 1.00 | 33.49 | B | C |
| ATOM | 3402 | C | ALA | B | 146 | −24.709 | −18.100 | −18.948 | 1.00 | 34.85 | B | C |
| ATOM | 3403 | O | ALA | B | 146 | −24.835 | −18.639 | −20.052 | 1.00 | 36.76 | B | O |
| ATOM | 3404 | N | GLY | B | 147 | −24.277 | −18.752 | −17.873 | 1.00 | 33.97 | B | N |
| ATOM | 3405 | CA | GLY | B | 147 | −23.841 | −20.144 | −17.930 | 1.00 | 32.43 | B | C |
| ATOM | 3406 | C | GLY | B | 147 | −22.627 | −20.388 | −18.804 | 1.00 | 31.88 | B | C |
| ATOM | 3407 | O | GLY | B | 147 | −22.585 | −21.373 | −19.541 | 1.00 | 32.47 | B | O |
| ATOM | 3408 | N | LEU | B | 148 | −21.632 | −19.506 | −18.744 | 1.00 | 32.61 | B | N |
| ATOM | 3409 | CA | LEU | B | 148 | −20.451 | −19.658 | −19.605 | 1.00 | 33.79 | B | C |
| ATOM | 3410 | CB | LEU | B | 148 | −19.475 | −18.492 | −19.444 | 1.00 | 35.60 | B | C |
| ATOM | 3411 | CG | LEU | B | 148 | −19.982 | −17.190 | −20.083 | 1.00 | 38.31 | B | C |
| ATOM | 3412 | CD1 | LEU | B | 148 | −19.402 | −15.964 | −19.399 | 1.00 | 37.11 | B | C |
| ATOM | 3413 | CD2 | LEU | B | 148 | −19.694 | −17.157 | −21.585 | 1.00 | 40.39 | B | C |
| ATOM | 3414 | C | LEU | B | 148 | −19.783 | −20.967 | −19.246 | 1.00 | 32.00 | B | C |
| ATOM | 3415 | O | LEU | B | 148 | −19.587 | −21.241 | −18.078 | 1.00 | 29.10 | B | O |
| ATOM | 3416 | N | GLU | B | 149 | −19.474 | −21.789 | −20.246 | 1.00 | 31.33 | B | N |
| ATOM | 3417 | CA | GLU | B | 149 | −18.940 | −23.146 | −19.978 | 1.00 | 29.16 | B | C |
| ATOM | 3418 | CB | GLU | B | 149 | −19.192 | −24.108 | −21.161 | 1.00 | 27.96 | B | C |
| ATOM | 3419 | CG | GLU | B | 149 | −20.666 | −24.425 | −21.298 | 1.00 | 26.85 | B | C |
| ATOM | 3420 | CD | GLU | B | 149 | −21.026 | −25.095 | −22.589 | 1.00 | 24.90 | B | C |
| ATOM | 3421 | OE1 | GLU | B | 149 | −22.188 | −25.536 | −22.681 | 1.00 | 23.70 | B | O |
| ATOM | 3422 | OE2 | GLU | B | 149 | −20.181 | −25.161 | −23.493 | 1.00 | 23.35 | B | O |
| ATOM | 3423 | C | GLU | B | 149 | −17.471 | −23.082 | −19.639 | 1.00 | 25.48 | B | C |
| ATOM | 3424 | O | GLU | B | 149 | −16.708 | −22.371 | −20.273 | 1.00 | 24.06 | B | O |
| ATOM | 3425 | N | TYR | B | 150 | −17.091 | −23.848 | −18.640 | 1.00 | 23.26 | B | N |
| ATOM | 3426 | CA | TYR | B | 150 | −15.795 | −23.690 | −18.059 | 1.00 | 23.09 | B | C |
| ATOM | 3427 | CB | TYR | B | 150 | −15.847 | −24.115 | −16.606 | 1.00 | 22.13 | B | C |
| ATOM | 3428 | CG | TYR | B | 150 | −14.796 | −23.476 | −15.770 | 1.00 | 22.41 | B | C |
| ATOM | 3429 | CD1 | TYR | B | 150 | −14.899 | −22.133 | −15.406 | 1.00 | 22.39 | B | C |
| ATOM | 3430 | CE1 | TYR | B | 150 | −13.920 | −21.529 | −14.620 | 1.00 | 22.34 | B | C |
| ATOM | 3431 | CZ | TYR | B | 150 | −12.828 | −22.277 | −14.176 | 1.00 | 22.55 | B | C |
| ATOM | 3432 | OH | TYR | B | 150 | −11.884 | −21.671 | −13.406 | 1.00 | 21.97 | B | O |
| ATOM | 3433 | CE2 | TYR | B | 150 | −12.697 | −23.623 | −14.518 | 1.00 | 22.68 | B | C |
| ATOM | 3434 | CD2 | TYR | B | 150 | −13.676 | −24.213 | −15.319 | 1.00 | 22.78 | B | C |
| ATOM | 3435 | C | TYR | B | 150 | −14.760 | −24.462 | −18.871 | 1.00 | 23.03 | B | C |
| ATOM | 3436 | O | TYR | B | 150 | −14.194 | −25.466 | −18.425 | 1.00 | 25.23 | B | O |
| ATOM | 3437 | N | TYR | B | 151 | −14.522 | −23.970 | −20.075 | 1.00 | 21.89 | B | N |
| ATOM | 3438 | CA | TYR | B | 151 | −13.540 | −24.533 | −20.963 | 1.00 | 22.19 | B | C |
| ATOM | 3439 | CB | TYR | B | 151 | −14.012 | −25.873 | −21.514 | 1.00 | 22.84 | B | C |
| ATOM | 3440 | CG | TYR | B | 151 | −12.869 | −26.737 | −21.992 | 1.00 | 24.39 | B | C |
| ATOM | 3441 | CD1 | TYR | B | 151 | −12.365 | −26.643 | −23.293 | 1.00 | 24.16 | B | C |
| ATOM | 3442 | CE1 | TYR | B | 151 | −11.298 | −27.439 | −23.704 | 1.00 | 23.84 | B | C |
| ATOM | 3443 | CZ | TYR | B | 151 | −10.738 | −28.326 | −22.820 | 1.00 | 24.46 | B | C |
| ATOM | 3444 | OH | TYR | B | 151 | −9.695 | −29.143 | −23.159 | 1.00 | 26.46 | B | O |
| ATOM | 3445 | CE2 | TYR | B | 151 | −11.214 | −28.432 | −21.538 | 1.00 | 25.25 | B | C |
| ATOM | 3446 | CD2 | TYR | B | 151 | −12.263 | −27.640 | −21.125 | 1.00 | 25.13 | B | C |
| ATOM | 3447 | C | TYR | B | 151 | −13.325 | −23.580 | −22.124 | 1.00 | 22.44 | B | C |
| ATOM | 3448 | O | TYR | B | 151 | −14.227 | −22.838 | −22.485 | 1.00 | 22.50 | B | O |
| ATOM | 3449 | N | GLY | B | 152 | −12.139 | −23.613 | −22.717 | 1.00 | 23.25 | B | N |
| ATOM | 3450 | CA | GLY | B | 152 | −11.812 | −22.762 | −23.866 | 1.00 | 24.07 | B | C |
| ATOM | 3451 | C | GLY | B | 152 | −12.135 | −21.280 | −23.685 | 1.00 | 24.84 | B | C |
| ATOM | 3452 | O | GLY | B | 152 | −12.043 | −20.733 | −22.564 | 1.00 | 25.07 | B | O |
| ATOM | 3453 | N | GLU | B | 153 | −12.539 | −20.635 | −24.787 | 1.00 | 25.04 | B | N |
| ATOM | 3454 | CA | GLU | B | 153 | −12.871 | −19.202 | −24.766 | 1.00 | 24.00 | B | C |
| ATOM | 3455 | CB | GLU | B | 153 | −13.149 | −18.678 | −26.183 | 1.00 | 23.45 | B | C |
| ATOM | 3456 | CG | GLU | B | 153 | −11.936 | −18.864 | −27.107 | 1.00 | 23.71 | B | C |
| ATOM | 3457 | CD | GLU | B | 153 | −11.913 | −17.978 | −28.361 | 1.00 | 24.91 | B | C |
| ATOM | 3458 | OE1 | GLU | B | 153 | −12.587 | −16.903 | −28.423 | 1.00 | 24.29 | B | O |
| ATOM | 3459 | OE2 | GLU | B | 153 | −11.174 | −18.362 | −29.304 | 1.00 | 24.99 | B | O |
| ATOM | 3460 | C | GLU | B | 153 | −14.005 | −18.909 | −23.771 | 1.00 | 23.93 | B | C |
| ATOM | 3461 | O | GLU | B | 153 | −13.861 | −18.016 | −22.931 | 1.00 | 23.19 | B | O |
| ATOM | 3462 | N | SER | B | 154 | −15.085 | −19.689 | −23.812 | 1.00 | 23.68 | B | N |
| ATOM | 3463 | CA | SER | B | 154 | −16.128 | −19.584 | −22.790 | 1.00 | 24.55 | B | C |
| ATOM | 3464 | CB | SER | B | 154 | −16.967 | −20.848 | −22.696 | 1.00 | 25.82 | B | C |
| ATOM | 3465 | OG | SER | B | 154 | −18.170 | −20.690 | −23.396 | 1.00 | 29.35 | B | O |
| ATOM | 3466 | C | SER | B | 154 | −15.567 | −19.381 | −21.419 | 1.00 | 24.67 | B | C |
| ATOM | 3467 | O | SER | B | 154 | −15.952 | −18.454 | −20.715 | 1.00 | 26.62 | B | O |
| ATOM | 3468 | N | GLY | B | 155 | −14.688 | −20.288 | −21.023 | 1.00 | 24.28 | B | N |
| ATOM | 3469 | CA | GLY | B | 155 | −14.237 | −20.370 | −19.635 | 1.00 | 24.21 | B | C |
| ATOM | 3470 | C | GLY | B | 155 | −13.255 | −19.268 | −19.266 | 1.00 | 24.50 | B | C |
| ATOM | 3471 | O | GLY | B | 155 | −13.162 | −18.878 | −18.081 | 1.00 | 24.34 | B | O |
| ATOM | 3472 | N | ALA | B | 156 | −12.518 | −18.776 | −20.272 | 1.00 | 22.74 | B | N |
| ATOM | 3473 | CA | ALA | B | 156 | −11.638 | −17.640 | −20.087 | 1.00 | 21.34 | B | C |
| ATOM | 3474 | CB | ALA | B | 156 | −10.756 | −17.444 | −21.302 | 1.00 | 21.61 | B | C |
| ATOM | 3475 | C | ALA | B | 156 | −12.447 | −16.379 | −19.758 | 1.00 | 20.58 | B | C |
| ATOM | 3476 | O | ALA | B | 156 | −12.066 | −15.588 | −18.881 | 1.00 | 19.77 | B | O |
| ATOM | 3477 | N | LEU | B | 157 | −13.573 | −16.213 | −20.435 | 1.00 | 20.76 | B | N |
| ATOM | 3478 | CA | LEU | B | 157 | −14.554 | −15.186 | −20.067 | 1.00 | 21.15 | B | C |
| ATOM | 3479 | CB | LEU | B | 157 | −15.696 | −15.131 | −21.091 | 1.00 | 22.03 | B | C |
| ATOM | 3480 | CG | LEU | B | 157 | −15.537 | −14.290 | −22.373 | 1.00 | 23.05 | B | C |
| ATOM | 3481 | CD1 | LEU | B | 157 | −14.127 | −14.275 | −22.957 | 1.00 | 23.70 | B | C |
| ATOM | 3482 | CD2 | LEU | B | 157 | −16.520 | −14.776 | −23.429 | 1.00 | 23.26 | B | C |
| ATOM | 3483 | C | LEU | B | 157 | −15.124 | −15.451 | −18.670 | 1.00 | 20.38 | B | C |
| ATOM | 3484 | O | LEU | B | 157 | −15.166 | −14.576 | −17.837 | 1.00 | 20.58 | B | O |
| ATOM | 3485 | N | ASN | B | 158 | −15.558 | −16.669 | −18.414 | 1.00 | 20.57 | B | N |
| ATOM | 3486 | CA | ASN | B | 158 | −16.143 | −17.009 | −17.122 | 1.00 | 20.45 | B | C |
| ATOM | 3487 | CB | ASN | B | 158 | −16.434 | −18.523 | −17.105 | 1.00 | 19.60 | B | C |
| ATOM | 3488 | CG | ASN | B | 158 | −16.948 | −19.017 | −15.773 | 1.00 | 18.40 | B | C |
| ATOM | 3489 | OD1 | ASN | B | 158 | −17.952 | −19.699 | −15.705 | 1.00 | 17.09 | B | O |
| ATOM | 3490 | ND2 | ASN | B | 158 | −16.260 | −18.667 | −14.711 | 1.00 | 18.42 | B | N |
| ATOM | 3491 | C | ASN | B | 158 | −15.205 | −16.580 | −15.964 | 1.00 | 21.47 | B | C |
| ATOM | 3492 | O | ASN | B | 158 | −15.643 | −15.988 | −14.948 | 1.00 | 21.75 | B | O |
| ATOM | 3493 | N | GLU | B | 159 | −13.923 | −16.919 | −16.124 | 1.00 | 21.70 | B | N |
| ATOM | 3494 | CA | GLU | B | 159 | −12.856 | −16.482 | −15.213 | 1.00 | 21.57 | B | C |
| ATOM | 3495 | CB | GLU | B | 159 | −11.542 | −17.158 | −15.612 | 1.00 | 20.39 | B | C |
| ATOM | 3496 | CG | GLU | B | 159 | −11.355 | −18.493 | −14.966 | 1.00 | 20.39 | B | C |
| ATOM | 3497 | CD | GLU | B | 159 | −11.302 | −18.353 | −13.467 | 1.00 | 21.79 | B | C |
| ATOM | 3498 | OE1 | GLU | B | 159 | −10.430 | −17.542 | −13.054 | 1.00 | 25.14 | B | O |
| ATOM | 3499 | OE2 | GLU | B | 159 | −12.105 | −18.981 | −12.712 | 1.00 | 20.07 | B | O |
| ATOM | 3500 | C | GLU | B | 159 | −12.692 | −14.940 | −15.195 | 1.00 | 23.60 | B | C |
| ATOM | 3501 | O | GLU | B | 159 | −12.857 | −14.289 | −14.147 | 1.00 | 22.28 | B | O |
| ATOM | 3502 | N | SER | B | 160 | −12.393 | −14.354 | −16.360 | 1.00 | 25.08 | B | N |
| ATOM | 3503 | CA | SER | B | 160 | −12.228 | −12.906 | −16.432 | 1.00 | 26.35 | B | C |
| ATOM | 3504 | CB | SER | B | 160 | −12.121 | −12.385 | −17.864 | 1.00 | 26.83 | B | C |
| ATOM | 3505 | OG | SER | B | 160 | −11.919 | −10.970 | −17.841 | 1.00 | 27.65 | B | O |
| ATOM | 3506 | C | SER | B | 160 | −13.362 | −12.181 | −15.740 | 1.00 | 26.39 | B | C |
| ATOM | 3507 | O | SER | B | 160 | −13.108 | −11.202 | −15.049 | 1.00 | 27.56 | B | O |
| ATOM | 3508 | N | ILE | B | 161 | −14.602 | −12.640 | −15.906 | 1.00 | 25.59 | B | N |
| ATOM | 3509 | CA | ILE | B | 161 | −15.716 | −11.934 | −15.265 | 1.00 | 25.62 | B | C |
| ATOM | 3510 | CB | ILE | B | 161 | −17.102 | −12.304 | −15.858 | 1.00 | 25.50 | B | C |
| ATOM | 3511 | CG1 | ILE | B | 161 | −17.410 | −11.343 | −17.010 | 1.00 | 26.10 | B | C |
| ATOM | 3512 | CD1 | ILE | B | 161 | −18.251 | −11.934 | −18.118 | 1.00 | 26.54 | B | C |
| ATOM | 3513 | CG2 | ILE | B | 161 | −18.232 | −12.168 | −14.845 | 1.00 | 25.32 | B | C |
| ATOM | 3514 | C | ILE | B | 161 | −15.633 | −12.060 | −13.752 | 1.00 | 24.63 | B | C |
| ATOM | 3515 | O | ILE | B | 161 | −15.751 | −11.057 | −13.032 | 1.00 | 25.26 | B | O |
| ATOM | 3516 | N | SER | B | 162 | −15.393 | −13.268 | −13.277 | 1.00 | 22.63 | B | N |
| ATOM | 3517 | CA | SER | B | 162 | −15.191 | −13.452 | −11.859 | 1.00 | 22.29 | B | C |
| ATOM | 3518 | CB | SER | B | 162 | −14.971 | −14.944 | −11.505 | 1.00 | 22.14 | B | C |
| ATOM | 3519 | OG | SER | B | 162 | −16.160 | −15.704 | −11.695 | 1.00 | 22.68 | B | O |
| ATOM | 3520 | C | SER | B | 162 | −14.026 | −12.561 | −11.365 | 1.00 | 21.42 | B | C |
| ATOM | 3521 | O | SER | B | 162 | −14.094 | −12.053 | −10.261 | 1.00 | 22.43 | B | O |
| ATOM | 3522 | N | ASP | B | 163 | −12.974 | −12.365 | −12.159 | 1.00 | 20.02 | B | N |
| ATOM | 3523 | CA | ASP | B | 163 | −11.870 | −11.480 | −11.757 | 1.00 | 20.14 | B | C |
| ATOM | 3524 | CB | ASP | B | 163 | −10.676 | −11.584 | −12.721 | 1.00 | 19.49 | B | C |
| ATOM | 3525 | CG | ASP | B | 163 | −9.815 | −12.793 | −12.467 | 1.00 | 18.52 | B | C |
| ATOM | 3526 | OD1 | ASP | B | 163 | −10.121 | −13.559 | −11.555 | 1.00 | 18.96 | B | O |
| ATOM | 3527 | OD2 | ASP | B | 163 | −8.819 | −12.979 | −13.170 | 1.00 | 17.18 | B | O |
| ATOM | 3528 | C | ASP | B | 163 | −12.306 | −10.011 | −11.726 | 1.00 | 21.75 | B | C |
| ATOM | 3529 | O | ASP | B | 163 | −11.987 | −9.257 | −10.797 | 1.00 | 22.65 | B | O |
| ATOM | 3530 | N | ILE | B | 164 | −13.027 | −9.594 | −12.758 | 1.00 | 22.58 | B | N |
| ATOM | 3531 | CA | ILE | B | 164 | −13.462 | −8.207 | −12.857 | 1.00 | 22.48 | B | C |
| ATOM | 3532 | CB | ILE | B | 164 | −14.299 | −7.988 | −14.114 | 1.00 | 22.54 | B | C |
| ATOM | 3533 | CG1 | ILE | B | 164 | −13.386 | −8.005 | −15.345 | 1.00 | 23.45 | B | C |
| ATOM | 3534 | CD1 | ILE | B | 164 | −14.066 | −8.416 | −16.632 | 1.00 | 23.65 | B | C |
| ATOM | 3535 | CG2 | ILE | B | 164 | −15.030 | −6.663 | −14.047 | 1.00 | 21.95 | B | C |
| ATOM | 3536 | C | ILE | B | 164 | −14.258 | −7.827 | −11.634 | 1.00 | 22.72 | B | C |
| ATOM | 3537 | O | ILE | B | 164 | −14.064 | −6.750 | −11.049 | 1.00 | 23.59 | B | O |
| ATOM | 3538 | N | ILE | B | 165 | −15.145 | −8.718 | −11.229 | 1.00 | 23.05 | B | N |
| ATOM | 3539 | CA | ILE | B | 165 | −16.010 | −8.405 | −10.127 | 1.00 | 23.88 | B | C |
| ATOM | 3540 | CB | ILE | B | 165 | −17.296 | −9.240 | −10.153 | 1.00 | 25.46 | B | C |
| ATOM | 3541 | CG1 | ILE | B | 165 | −18.236 | −8.725 | −11.258 | 1.00 | 25.84 | B | C |
| ATOM | 3542 | CD1 | ILE | B | 165 | −19.692 | −9.093 | −11.047 | 1.00 | 25.86 | B | C |
| ATOM | 3543 | CG2 | ILE | B | 165 | −17.987 | −9.179 | −8.793 | 1.00 | 26.66 | B | C |
| ATOM | 3544 | C | ILE | B | 165 | −15.281 | −8.569 | −8.814 | 1.00 | 23.03 | B | C |
| ATOM | 3545 | O | ILE | B | 165 | −15.446 | −7.770 | −7.914 | 1.00 | 23.61 | B | O |
| ATOM | 3546 | N | GLY | B | 166 | −14.475 | −9.601 | −8.693 | 1.00 | 22.77 | B | N |
| ATOM | 3547 | CA | GLY | B | 166 | −13.811 | −9.838 | −7.437 | 1.00 | 23.61 | B | C |
| ATOM | 3548 | C | GLY | B | 166 | −12.910 | −8.666 | −7.100 | 1.00 | 24.48 | B | C |
| ATOM | 3549 | O | GLY | B | 166 | −12.834 | −8.213 | −5.949 | 1.00 | 24.77 | B | O |
| ATOM | 3550 | N | ASN | B | 167 | −12.218 | −8.185 | −8.122 | 1.00 | 24.92 | B | N |
| ATOM | 3551 | CA | ASN | B | 167 | −11.332 | −7.061 | −7.974 | 1.00 | 25.37 | B | C |
| ATOM | 3552 | CB | ASN | B | 167 | −10.538 | −6.834 | −9.250 | 1.00 | 25.09 | B | C |
| ATOM | 3553 | CG | ASN | B | 167 | −9.722 | −5.578 | −9.179 | 1.00 | 24.84 | B | C |
| ATOM | 3554 | OD1 | ASN | B | 167 | −8.663 | −5.557 | −8.554 | 1.00 | 24.32 | B | O |
| ATOM | 3555 | ND2 | ASN | B | 167 | −10.223 | −4.509 | −9.792 | 1.00 | 25.27 | B | N |
| ATOM | 3556 | C | ASN | B | 167 | −12.098 | −5.789 | −7.648 | 1.00 | 25.52 | B | C |
| ATOM | 3557 | O | ASN | B | 167 | −11.629 | −4.960 | −6.876 | 1.00 | 27.09 | B | O |
| ATOM | 3558 | N | ALA | B | 168 | −13.262 | −5.636 | −8.258 | 1.00 | 25.33 | B | N |
| ATOM | 3559 | CA | ALA | B | 168 | −14.077 | −4.456 | −8.054 | 1.00 | 26.46 | B | C |
| ATOM | 3560 | CB | ALA | B | 168 | −15.209 | −4.423 | −9.078 | 1.00 | 26.58 | B | C |
| ATOM | 3561 | C | ALA | B | 168 | −14.627 | −4.372 | −6.633 | 1.00 | 27.05 | B | C |
| ATOM | 3562 | O | ALA | B | 168 | −14.769 | −3.305 | −6.099 | 1.00 | 25.94 | B | O |
| ATOM | 3563 | N | ILE | B | 169 | −14.948 | −5.496 | −6.021 | 1.00 | 30.59 | B | N |
| ATOM | 3564 | CA | ILE | B | 169 | −15.426 | −5.488 | −4.640 | 1.00 | 34.83 | B | C |
| ATOM | 3565 | CB | ILE | B | 169 | −15.783 | −6.925 | −4.196 | 1.00 | 37.29 | B | C |
| ATOM | 3566 | CG1 | ILE | B | 169 | −17.197 | −7.244 | −4.697 | 1.00 | 40.64 | B | C |
| ATOM | 3567 | CD1 | ILE | B | 169 | −17.388 | −8.660 | −5.177 | 1.00 | 41.49 | B | C |
| ATOM | 3568 | CG2 | ILE | B | 169 | −15.710 | −7.094 | −2.681 | 1.00 | 36.37 | B | C |
| ATOM | 3569 | C | ILE | B | 169 | −14.388 | −4.882 | −3.715 | 1.00 | 36.99 | B | C |
| ATOM | 3570 | O | ILE | B | 169 | −14.662 | −3.984 | −2.918 | 1.00 | 33.86 | B | O |
| ATOM | 3571 | N | ASP | B | 170 | −13.186 | −5.413 | −3.841 | 1.00 | 43.53 | B | N |
| ATOM | 3572 | CA | ASP | B | 170 | −12.072 | −5.011 | −3.016 | 1.00 | 48.08 | B | C |
| ATOM | 3573 | CB | ASP | B | 170 | −10.814 | −5.803 | −3.463 | 1.00 | 51.67 | B | C |
| ATOM | 3574 | CG | ASP | B | 170 | −9.755 | −5.980 | −2.344 | 1.00 | 52.92 | B | C |
| ATOM | 3575 | OD1 | ASP | B | 170 | −9.729 | −5.192 | −1.351 | 1.00 | 50.54 | B | O |
| ATOM | 3576 | OD2 | ASP | B | 170 | −8.931 | −6.919 | −2.499 | 1.00 | 50.26 | B | O |
| ATOM | 3577 | C | ASP | B | 170 | −11.919 | −3.483 | −3.142 | 1.00 | 44.26 | B | C |
| ATOM | 3578 | O | ASP | B | 170 | −12.078 | −2.785 | −2.164 | 1.00 | 43.51 | B | O |
| ATOM | 3579 | N | GLY | B | 171 | −11.672 | −2.990 | −4.358 | 1.00 | 43.91 | B | N |
| ATOM | 3580 | CA | GLY | B | 171 | −11.467 | −1.557 | −4.634 | 1.00 | 44.24 | B | C |
| ATOM | 3581 | C | GLY | B | 171 | −10.003 | −1.160 | −4.820 | 1.00 | 44.95 | B | C |
| ATOM | 3582 | O | GLY | B | 171 | −9.634 | −0.569 | −5.831 | 1.00 | 43.83 | B | O |
| ATOM | 3583 | N | LYS | B | 172 | −9.194 | −1.498 | −3.815 | 1.00 | 46.20 | B | N |
| ATOM | 3584 | CA | LYS | B | 172 | −7.749 | −1.197 | −3.709 | 1.00 | 46.62 | B | C |
| ATOM | 3585 | CB | LYS | B | 172 | −7.096 | −2.285 | −2.836 | 1.00 | 52.15 | B | C |
| ATOM | 3586 | CG | LYS | B | 172 | −7.272 | −2.161 | −1.319 | 1.00 | 57.60 | B | C |
| ATOM | 3587 | CD | LYS | B | 172 | −6.481 | −3.256 | −0.557 | 1.00 | 62.15 | B | C |
| ATOM | 3588 | CE | LYS | B | 172 | −4.946 | −3.091 | −0.575 | 1.00 | 60.28 | B | C |
| ATOM | 3589 | NZ | LYS | B | 172 | −4.444 | −1.991 | 0.304 | 1.00 | 61.46 | B | N |
| ATOM | 3590 | C | LYS | B | 172 | −6.850 | −1.027 | −4.980 | 1.00 | 42.61 | B | C |
| ATOM | 3591 | O | LYS | B | 172 | −6.188 | −0.001 | −5.125 | 1.00 | 42.17 | B | O |
| ATOM | 3592 | N | ASN | B | 173 | −6.769 | −2.039 | −5.854 | 1.00 | 36.61 | B | N |
| ATOM | 3593 | CA | ASN | B | 173 | −5.709 | −2.089 | −6.899 | 1.00 | 30.24 | B | C |
| ATOM | 3594 | CB | ASN | B | 173 | −4.433 | −2.744 | −6.350 | 1.00 | 28.81 | B | C |
| ATOM | 3595 | CG | ASN | B | 173 | −4.692 | −4.072 | −5.650 | 1.00 | 29.12 | B | C |
| ATOM | 3596 | OD1 | ASN | B | 173 | −5.413 | −4.949 | −6.145 | 1.00 | 30.32 | B | O |
| ATOM | 3597 | ND2 | ASN | B | 173 | −4.092 | −4.228 | −4.481 | 1.00 | 28.75 | B | N |
| ATOM | 3598 | C | ASN | B | 173 | −6.168 | −2.796 | −8.164 | 1.00 | 26.96 | B | C |
| ATOM | 3599 | O | ASN | B | 173 | −7.342 | −3.036 | −8.324 | 1.00 | 24.81 | B | O |
| ATOM | 3600 | N | TRP | B | 174 | −5.241 | −3.078 | −9.070 | 1.00 | 25.49 | B | N |
| ATOM | 3601 | CA | TRP | B | 174 | −5.513 | −3.837 | −10.283 | 1.00 | 25.26 | B | C |
| ATOM | 3602 | CB | TRP | B | 174 | −4.795 | −3.193 | −11.470 | 1.00 | 25.93 | B | C |
| ATOM | 3603 | CG | TRP | B | 174 | −5.293 | −1.836 | −11.778 | 1.00 | 26.89 | B | C |
| ATOM | 3604 | CD1 | TRP | B | 174 | −4.593 | −0.665 | −11.679 | 1.00 | 26.32 | B | C |
| ATOM | 3605 | NE1 | TRP | B | 174 | −5.397 | 0.382 | −12.017 | 1.00 | 26.11 | B | N |
| ATOM | 3606 | CE2 | TRP | B | 174 | −6.637 | −0.087 | −12.361 | 1.00 | 26.19 | B | C |
| ATOM | 3607 | CD2 | TRP | B | 174 | −6.607 | −1.487 | −12.217 | 1.00 | 26.18 | B | C |
| ATOM | 3608 | CE3 | TRP | B | 174 | −7.761 | −2.215 | −12.487 | 1.00 | 26.75 | B | C |
| ATOM | 3609 | CZ3 | TRP | B | 174 | −8.904 | −1.520 | −12.907 | 1.00 | 28.43 | B | C |
| ATOM | 3610 | CH2 | TRP | B | 174 | −8.895 | −0.122 | −13.051 | 1.00 | 26.84 | B | C |
| ATOM | 3611 | CZ2 | TRP | B | 174 | −7.777 | 0.606 | −12.787 | 1.00 | 26.52 | B | C |
| ATOM | 3612 | C | TRP | B | 174 | −5.052 | −5.296 | −10.209 | 1.00 | 24.68 | B | C |
| ATOM | 3613 | O | TRP | B | 174 | −4.843 | −5.937 | −11.257 | 1.00 | 24.26 | B | O |
| ATOM | 3614 | N | LEU | B | 175 | −4.878 | −5.827 | −9.001 | 1.00 | 22.75 | B | N |
| ATOM | 3615 | CA | LEU | B | 175 | −4.318 | −7.160 | −8.859 | 1.00 | 21.88 | B | C |
| ATOM | 3616 | CB | LEU | B | 175 | −3.150 | −7.131 | −7.894 | 1.00 | 20.46 | B | C |
| ATOM | 3617 | CG | LEU | B | 175 | −2.144 | −5.994 | −8.090 | 1.00 | 19.93 | B | C |
| ATOM | 3618 | CD1 | LEU | B | 175 | −1.194 | −5.955 | −6.916 | 1.00 | 20.20 | B | C |
| ATOM | 3619 | CD2 | LEU | B | 175 | −1.336 | −6.095 | −9.364 | 1.00 | 19.56 | B | C |
| ATOM | 3620 | C | LEU | B | 175 | −5.389 | −8.154 | −8.417 | 1.00 | 22.98 | B | C |
| ATOM | 3621 | O | LEU | B | 175 | −6.489 | −7.765 | −8.000 | 1.00 | 23.17 | B | O |
| ATOM | 3622 | N | ILE | B | 176 | −5.065 | −9.439 | −8.541 | 1.00 | 24.28 | B | N |
| ATOM | 3623 | CA | ILE | B | 176 | −5.893 | −10.524 | −7.968 | 1.00 | 24.99 | B | C |
| ATOM | 3624 | CB | ILE | B | 176 | −6.531 | −11.412 | −9.059 | 1.00 | 26.10 | B | C |
| ATOM | 3625 | CG1 | ILE | B | 176 | −7.357 | −10.570 | −10.055 | 1.00 | 26.43 | B | C |
| ATOM | 3626 | CD1 | ILE | B | 176 | −8.802 | −10.335 | −9.660 | 1.00 | 26.54 | B | C |
| ATOM | 3627 | CG2 | ILE | B | 176 | −7.375 | −12.507 | −8.413 | 1.00 | 26.92 | B | C |
| ATOM | 3628 | C | ILE | B | 176 | −5.044 | −11.412 | −7.040 | 1.00 | 23.20 | B | C |
| ATOM | 3629 | O | ILE | B | 176 | −3.889 | −11.741 | −7.341 | 1.00 | 21.47 | B | O |
| ATOM | 3630 | N | GLY | B | 177 | −5.620 | −11.773 | −5.902 | 1.00 | 22.18 | B | N |
| ATOM | 3631 | CA | GLY | B | 177 | −4.925 | −12.603 | −4.925 | 1.00 | 21.76 | B | C |
| ATOM | 3632 | C | GLY | B | 177 | −3.641 | −11.978 | −4.396 | 1.00 | 20.91 | B | C |
| ATOM | 3633 | O | GLY | B | 177 | −2.768 | −12.680 | −3.908 | 1.00 | 19.00 | B | O |
| ATOM | 3634 | N | ASP | B | 178 | −3.528 | −10.652 | −4.488 | 1.00 | 21.50 | B | N |
| ATOM | 3635 | CA | ASP | B | 178 | −2.352 | −9.928 | −3.952 | 1.00 | 20.88 | B | C |
| ATOM | 3636 | CB | ASP | B | 178 | −2.447 | −8.386 | −4.153 | 1.00 | 20.48 | B | C |
| ATOM | 3637 | CG | ASP | B | 178 | −3.764 | −7.757 | −3.622 | 1.00 | 20.78 | B | C |
| ATOM | 3638 | OD1 | ASP | B | 178 | −4.882 | −8.233 | −3.951 | 1.00 | 21.09 | B | O |
| ATOM | 3639 | OD2 | ASP | B | 178 | −3.680 | −6.747 | −2.891 | 1.00 | 20.74 | B | O |
| ATOM | 3640 | C | ASP | B | 178 | −2.109 | −10.303 | −2.482 | 1.00 | 20.38 | B | C |
| ATOM | 3641 | O | ASP | B | 178 | −0.951 | −10.556 | −2.090 | 1.00 | 21.24 | B | O |
| ATOM | 3642 | N | LEU | B | 179 | −3.198 | −10.400 | −1.711 | 1.00 | 18.74 | B | N |
| ATOM | 3643 | CA | LEU | B | 179 | −3.130 | −10.728 | −0.287 | 1.00 | 18.78 | B | C |
| ATOM | 3644 | CB | LEU | B | 179 | −4.449 | −10.345 | 0.395 | 1.00 | 18.48 | B | C |
| ATOM | 3645 | CG | LEU | B | 179 | −4.697 | −8.834 | 0.495 | 1.00 | 18.11 | B | C |
| ATOM | 3646 | CD1 | LEU | B | 179 | −5.946 | −8.555 | 1.305 | 1.00 | 17.54 | B | C |
| ATOM | 3647 | CD2 | LEU | B | 179 | −3.488 | −8.118 | 1.091 | 1.00 | 17.78 | B | C |
| ATOM | 3648 | C | LEU | B | 179 | −2.776 | −12.178 | 0.103 | 1.00 | 18.56 | B | C |
| ATOM | 3649 | O | LEU | B | 179 | −2.211 | −12.399 | 1.176 | 1.00 | 20.52 | B | O |
| ATOM | 3650 | N | ILE | B | 180 | −3.101 | −13.155 | −0.735 | 1.00 | 17.63 | B | N |
| ATOM | 3651 | CA | ILE | B | 180 | −2.873 | −14.586 | −0.399 | 1.00 | 17.04 | B | C |
| ATOM | 3652 | CB | ILE | B | 180 | −4.068 | −15.475 | −0.767 | 1.00 | 15.77 | B | C |
| ATOM | 3653 | CG1 | ILE | B | 180 | −4.340 | −15.399 | −2.264 | 1.00 | 15.25 | B | C |
| ATOM | 3654 | CD1 | ILE | B | 180 | −5.519 | −16.216 | −2.719 | 1.00 | 15.28 | B | C |
| ATOM | 3655 | CG2 | ILE | B | 180 | −5.267 | −15.102 | 0.092 | 1.00 | 15.84 | B | C |
| ATOM | 3656 | C | ILE | B | 180 | −1.628 | −15.229 | −1.033 | 1.00 | 17.27 | B | C |
| ATOM | 3657 | O | ILE | B | 180 | −1.215 | −16.305 | −0.630 | 1.00 | 16.09 | B | O |
| ATOM | 3658 | N | TYR | B | 181 | −1.032 | −14.546 | −1.996 | 1.00 | 17.64 | B | N |
| ATOM | 3659 | CA | TYR | B | 181 | −0.004 | −15.128 | −2.796 | 1.00 | 18.26 | B | C |
| ATOM | 3660 | CB | TYR | B | 181 | −0.095 | −14.544 | −4.179 | 1.00 | 17.80 | B | C |
| ATOM | 3661 | CG | TYR | B | 181 | 0.789 | −15.211 | −5.177 | 1.00 | 17.24 | B | C |
| ATOM | 3662 | CD1 | TYR | B | 181 | 0.678 | −16.565 | −5.429 | 1.00 | 16.90 | B | C |
| ATOM | 3663 | CE1 | TYR | B | 181 | 1.461 | −17.182 | −6.364 | 1.00 | 16.27 | B | C |
| ATOM | 3664 | CZ | TYR | B | 181 | 2.349 | −16.457 | −7.064 | 1.00 | 16.18 | B | C |
| ATOM | 3665 | OH | TYR | B | 181 | 3.129 | −17.077 | −7.986 | 1.00 | 16.18 | B | O |
| ATOM | 3666 | CE2 | TYR | B | 181 | 2.477 | −15.117 | −6.853 | 1.00 | 16.98 | B | C |
| ATOM | 3667 | CD2 | TYR | B | 181 | 1.693 | −14.495 | −5.905 | 1.00 | 16.99 | B | C |
| ATOM | 3668 | C | TYR | B | 181 | 1.357 | −14.831 | −2.207 | 1.00 | 20.02 | B | C |
| ATOM | 3669 | O | TYR | B | 181 | 1.564 | −13.757 | −1.650 | 1.00 | 20.40 | B | O |
| ATOM | 3670 | N | THR | B | 182 | 2.255 | −15.812 | −2.327 | 1.00 | 22.26 | B | N |
| ATOM | 3671 | CA | THR | B | 182 | 3.651 | −15.730 | −1.896 | 1.00 | 25.08 | B | C |
| ATOM | 3672 | CB | THR | B | 182 | 4.512 | −15.160 | −3.027 | 1.00 | 24.99 | B | C |
| ATOM | 3673 | OG1 | THR | B | 182 | 3.846 | −14.005 | −3.582 | 1.00 | 24.72 | B | O |
| ATOM | 3674 | CG2 | THR | B | 182 | 4.758 | −16.227 | −4.105 | 1.00 | 24.58 | B | C |
| ATOM | 3675 | C | THR | B | 182 | 3.832 | −14.850 | −0.671 | 1.00 | 28.60 | B | C |
| ATOM | 3676 | O | THR | B | 182 | 4.283 | −13.693 | −0.784 | 1.00 | 30.04 | B | O |
| ATOM | 3677 | N | PRO | B | 183 | 3.456 | −15.368 | 0.504 | 1.00 | 31.17 | B | N |
| ATOM | 3678 | CA | PRO | B | 183 | 3.595 | −14.570 | 1.747 | 1.00 | 31.48 | B | C |
| ATOM | 3679 | CB | PRO | B | 183 | 3.080 | −15.521 | 2.843 | 1.00 | 30.90 | B | C |
| ATOM | 3680 | CG | PRO | B | 183 | 3.126 | −16.903 | 2.223 | 1.00 | 31.55 | B | C |
| ATOM | 3681 | CD | PRO | B | 183 | 2.875 | −16.701 | 0.756 | 1.00 | 31.17 | B | C |
| ATOM | 3682 | C | PRO | B | 183 | 5.033 | −14.084 | 2.022 | 1.00 | 29.96 | B | C |
| ATOM | 3683 | O | PRO | B | 183 | 5.213 | −13.051 | 2.626 | 1.00 | 28.64 | B | O |
| ATOM | 3684 | N | ASN | B | 184 | 6.034 | −14.795 | 1.531 | 1.00 | 31.82 | B | N |
| ATOM | 3685 | CA | ASN | B | 184 | 7.418 | −14.330 | 1.641 | 1.00 | 34.54 | B | C |
| ATOM | 3686 | CB | ASN | B | 184 | 8.349 | −15.531 | 1.782 | 1.00 | 37.59 | B | C |
| ATOM | 3687 | CG | ASN | B | 184 | 8.493 | −15.958 | 3.219 | 1.00 | 40.66 | B | C |
| ATOM | 3688 | OD1 | ASN | B | 184 | 7.511 | −16.023 | 3.964 | 1.00 | 43.19 | B | O |
| ATOM | 3689 | ND2 | ASN | B | 184 | 9.726 | −16.202 | 3.637 | 1.00 | 43.92 | B | N |
| ATOM | 3690 | C | ASN | B | 184 | 7.932 | −13.407 | 0.529 | 1.00 | 33.21 | B | C |
| ATOM | 3691 | O | ASN | B | 184 | 9.088 | −13.008 | 0.557 | 1.00 | 31.64 | B | O |
| ATOM | 3692 | N | THR | B | 185 | 7.072 | −13.065 | −0.431 | 1.00 | 32.76 | B | N |
| ATOM | 3693 | CA | THR | B | 185 | 7.391 | −12.095 | −1.489 | 1.00 | 29.86 | B | C |
| ATOM | 3694 | CB | THR | B | 185 | 7.364 | −12.773 | −2.865 | 1.00 | 28.78 | B | C |
| ATOM | 3695 | OG1 | THR | B | 185 | 8.096 | −14.008 | −2.838 | 1.00 | 26.37 | B | O |
| ATOM | 3696 | CG2 | THR | B | 185 | 7.922 | −11.871 | −3.926 | 1.00 | 28.45 | B | C |
| ATOM | 3697 | C | THR | B | 185 | 6.331 | −10.995 | −1.522 | 1.00 | 30.62 | B | C |
| ATOM | 3698 | O | THR | B | 185 | 5.205 | −11.248 | −1.957 | 1.00 | 29.21 | B | O |
| ATOM | 3699 | N | PRO | B | 186 | 6.666 | −9.783 | −1.033 | 1.00 | 33.29 | B | N |
| ATOM | 3700 | CA | PRO | B | 186 | 5.722 | −8.657 | −1.130 | 1.00 | 32.23 | B | C |
| ATOM | 3701 | CB | PRO | B | 186 | 6.304 | −7.611 | −0.161 | 1.00 | 33.18 | B | C |
| ATOM | 3702 | CG | PRO | B | 186 | 7.766 | −7.921 | −0.084 | 1.00 | 32.85 | B | C |
| ATOM | 3703 | CD | PRO | B | 186 | 7.839 | −9.429 | −0.197 | 1.00 | 34.69 | B | C |
| ATOM | 3704 | C | PRO | B | 186 | 5.660 | −8.066 | −2.522 | 1.00 | 30.17 | B | C |
| ATOM | 3705 | O | PRO | B | 186 | 6.550 | −8.322 | −3.337 | 1.00 | 28.22 | B | O |
| ATOM | 3706 | N | GLY | B | 187 | 4.596 | −7.298 | −2.778 | 1.00 | 28.51 | B | N |
| ATOM | 3707 | CA | GLY | B | 187 | 4.409 | −6.578 | −4.045 | 1.00 | 27.51 | B | C |
| ATOM | 3708 | C | GLY | B | 187 | 3.834 | −7.367 | −5.220 | 1.00 | 26.61 | B | C |
| ATOM | 3709 | O | GLY | B | 187 | 3.282 | −6.772 | −6.151 | 1.00 | 24.33 | B | O |
| ATOM | 3710 | N | ASP | B | 188 | 3.956 | −8.702 | −5.198 | 1.00 | 25.58 | B | N |
| ATOM | 3711 | CA | ASP | B | 188 | 3.484 | −9.510 | −6.321 | 1.00 | 23.74 | B | C |
| ATOM | 3712 | CB | ASP | B | 188 | 4.340 | −10.765 | −6.539 | 1.00 | 23.72 | B | C |
| ATOM | 3713 | CG | ASP | B | 188 | 4.212 | −11.774 | −5.429 | 1.00 | 24.47 | B | C |
| ATOM | 3714 | OD1 | ASP | B | 188 | 3.517 | −11.480 | −4.436 | 1.00 | 26.68 | B | O |
| ATOM | 3715 | OD2 | ASP | B | 188 | 4.805 | −12.876 | −5.549 | 1.00 | 23.28 | B | O |
| ATOM | 3716 | C | ASP | B | 188 | 2.006 | −9.823 | −6.175 | 1.00 | 22.61 | B | C |
| ATOM | 3717 | O | ASP | B | 188 | 1.335 | −9.343 | −5.259 | 1.00 | 22.22 | B | O |
| ATOM | 3718 | N | ALA | B | 189 | 1.502 | −10.584 | −7.135 | 1.00 | 22.07 | B | N |
| ATOM | 3719 | CA | ALA | B | 189 | 0.119 | −10.970 | −7.179 | 1.00 | 21.72 | B | C |
| ATOM | 3720 | CB | ALA | B | 189 | −0.686 | −9.866 | −7.814 | 1.00 | 21.95 | B | C |
| ATOM | 3721 | C | ALA | B | 189 | −0.063 | −12.285 | −7.949 | 1.00 | 21.99 | B | C |
| ATOM | 3722 | O | ALA | B | 189 | 0.894 | −12.860 | −8.477 | 1.00 | 21.72 | B | O |
| ATOM | 3723 | N | LEU | B | 190 | −1.299 | −12.772 | −7.982 | 1.00 | 22.12 | B | N |
| ATOM | 3724 | CA | LEU | B | 190 | −1.603 | −14.002 | −8.679 | 1.00 | 22.30 | B | C |
| ATOM | 3725 | CB | LEU | B | 190 | −2.900 | −14.600 | −8.147 | 1.00 | 21.89 | B | C |
| ATOM | 3726 | CG | LEU | B | 190 | −3.193 | −16.011 | −8.652 | 1.00 | 21.53 | B | C |
| ATOM | 3727 | CD1 | LEU | B | 190 | −2.019 | −16.923 | −8.379 | 1.00 | 21.51 | B | C |
| ATOM | 3728 | CD2 | LEU | B | 190 | −4.431 | −16.549 | −7.978 | 1.00 | 22.08 | B | C |
| ATOM | 3729 | C | LEU | B | 190 | −1.723 | −13.721 | −10.171 | 1.00 | 23.63 | B | C |
| ATOM | 3730 | O | LEU | B | 190 | −1.127 | −14.407 | −11.028 | 1.00 | 23.87 | B | O |
| ATOM | 3731 | N | ARG | B | 191 | −2.521 | −12.709 | −10.474 | 1.00 | 24.40 | B | N |
| ATOM | 3732 | CA | ARG | B | 191 | −2.519 | −12.133 | −11.801 | 1.00 | 24.81 | B | C |
| ATOM | 3733 | CB | ARG | B | 191 | −3.435 | −12.925 | −12.755 | 1.00 | 24.95 | B | C |
| ATOM | 3734 | CG | ARG | B | 191 | −4.751 | −13.372 | −12.166 | 1.00 | 24.69 | B | C |
| ATOM | 3735 | CD | ARG | B | 191 | −5.454 | −14.478 | −12.961 | 1.00 | 22.98 | B | C |
| ATOM | 3736 | NE | ARG | B | 191 | −6.713 | −14.712 | −12.268 | 1.00 | 21.71 | B | N |
| ATOM | 3737 | CZ | ARG | B | 191 | −6.947 | −15.702 | −11.423 | 1.00 | 22.56 | B | C |
| ATOM | 3738 | NH1 | ARG | B | 191 | −6.055 | −16.663 | −11.206 | 1.00 | 22.99 | B | N |
| ATOM | 3739 | NH2 | ARG | B | 191 | −8.121 | −15.770 | −10.827 | 1.00 | 24.08 | B | N |
| ATOM | 3740 | C | ARG | B | 191 | −2.815 | −10.632 | −11.748 | 1.00 | 23.88 | B | C |
| ATOM | 3741 | O | ARG | B | 191 | −3.248 | −10.103 | −10.707 | 1.00 | 23.94 | B | O |
| ATOM | 3742 | N | SER | B | 192 | −2.488 | −9.956 | −12.850 | 1.00 | 22.79 | B | N |
| ATOM | 3743 | CA | SER | B | 192 | −2.641 | −8.514 | −12.966 | 1.00 | 22.71 | B | C |
| ATOM | 3744 | CB | SER | B | 192 | −1.283 | −7.836 | −13.169 | 1.00 | 22.27 | B | C |
| ATOM | 3745 | OG | SER | B | 192 | −1.397 | −6.423 | −12.982 | 1.00 | 21.78 | B | O |
| ATOM | 3746 | C | SER | B | 192 | −3.534 | −8.165 | −14.134 | 1.00 | 22.71 | B | C |
| ATOM | 3747 | O | SER | B | 192 | −3.287 | −8.605 | −15.252 | 1.00 | 22.91 | B | O |
| ATOM | 3748 | N | MET | B | 193 | −4.548 | −7.347 | −13.877 | 1.00 | 23.42 | B | N |
| ATOM | 3749 | CA | MET | B | 193 | −5.441 | −6.863 | −14.932 | 1.00 | 24.44 | B | C |
| ATOM | 3750 | CB | MET | B | 193 | −6.750 | −6.308 | −14.362 | 1.00 | 24.83 | B | C |
| ATOM | 3751 | CG | MET | B | 193 | −7.378 | −7.179 | −13.277 | 1.00 | 26.13 | B | C |
| ATOM | 3752 | SD | MET | B | 193 | −9.085 | −6.776 | −12.861 | 1.00 | 28.08 | B | S |
| ATOM | 3753 | CE | MET | B | 193 | −9.889 | −7.597 | −14.223 | 1.00 | 28.27 | B | C |
| ATOM | 3754 | C | MET | B | 193 | −4.739 | −5.779 | −15.723 | 1.00 | 25.15 | B | C |
| ATOM | 3755 | O | MET | B | 193 | −5.018 | −5.604 | −16.894 | 1.00 | 27.54 | B | O |
| ATOM | 3756 | N | GLU | B | 194 | −3.823 | −5.059 | −15.087 | 1.00 | 25.44 | B | N |
| ATOM | 3757 | CA | GLU | B | 194 | −3.224 | −3.908 | −15.697 | 1.00 | 25.92 | B | C |
| ATOM | 3758 | CB | GLU | B | 194 | −2.674 | −2.962 | −14.636 | 1.00 | 27.83 | B | C |
| ATOM | 3759 | CG | GLU | B | 194 | −1.974 | −1.743 | −15.226 | 1.00 | 30.63 | B | C |
| ATOM | 3760 | CD | GLU | B | 194 | −0.940 | −1.142 | −14.304 | 1.00 | 32.59 | B | C |
| ATOM | 3761 | OE1 | GLU | B | 194 | −1.326 | −0.320 | −13.455 | 1.00 | 33.88 | B | O |
| ATOM | 3762 | OE2 | GLU | B | 194 | 0.256 | −1.487 | −14.441 | 1.00 | 34.79 | B | O |
| ATOM | 3763 | C | GLU | B | 194 | −2.129 | −4.387 | −16.641 | 1.00 | 25.90 | B | C |
| ATOM | 3764 | O | GLU | B | 194 | −2.014 | −3.896 | −17.758 | 1.00 | 26.66 | B | O |
| ATOM | 3765 | N | ASN | B | 195 | −1.333 | −5.351 | −16.206 | 1.00 | 24.42 | B | N |
| ATOM | 3766 | CA | ASN | B | 195 | −0.279 | −5.876 | −17.042 | 1.00 | 24.87 | B | C |
| ATOM | 3767 | CB | ASN | B | 195 | 1.032 | −5.137 | −16.742 | 1.00 | 26.34 | B | C |
| ATOM | 3768 | CG | ASN | B | 195 | 2.250 | −5.738 | −17.467 | 1.00 | 29.35 | B | C |
| ATOM | 3769 | OD1 | ASN | B | 195 | 2.153 | −6.336 | −18.562 | 1.00 | 29.51 | B | O |
| ATOM | 3770 | ND2 | ASN | B | 195 | 3.419 | −5.591 | −16.834 | 1.00 | 31.30 | B | N |
| ATOM | 3771 | C | ASN | B | 195 | −0.196 | −7.413 | −16.888 | 1.00 | 24.24 | B | C |
| ATOM | 3772 | O | ASN | B | 195 | 0.600 | −7.933 | −16.101 | 1.00 | 23.20 | B | O |
| ATOM | 3773 | N | PRO | B | 196 | −1.071 | −8.139 | −17.621 | 1.00 | 24.01 | B | N |
| ATOM | 3774 | CA | PRO | B | 196 | −1.135 | −9.606 | −17.577 | 1.00 | 23.24 | B | C |
| ATOM | 3775 | CB | PRO | B | 196 | −2.134 | −9.929 | −18.685 | 1.00 | 23.91 | B | C |
| ATOM | 3776 | CG | PRO | B | 196 | −3.074 | −8.754 | −18.693 | 1.00 | 23.50 | B | C |
| ATOM | 3777 | CD | PRO | B | 196 | −2.200 | −7.569 | −18.399 | 1.00 | 23.71 | B | C |
| ATOM | 3778 | C | PRO | B | 196 | 0.185 | −10.321 | −17.826 | 1.00 | 22.53 | B | C |
| ATOM | 3779 | O | PRO | B | 196 | 0.494 | −11.282 | −17.144 | 1.00 | 21.13 | B | O |
| ATOM | 3780 | N | LYS | B | 197 | 0.974 | −9.836 | −18.774 | 1.00 | 23.69 | B | N |
| ATOM | 3781 | CA | LYS | B | 197 | 2.289 | −10.419 | −19.040 | 1.00 | 24.36 | B | C |
| ATOM | 3782 | CB | LYS | B | 197 | 3.051 | −9.624 | −20.106 | 1.00 | 26.48 | B | C |
| ATOM | 3783 | CG | LYS | B | 197 | 2.522 | −9.758 | −21.531 | 1.00 | 28.64 | B | C |
| ATOM | 3784 | CD | LYS | B | 197 | 3.512 | −9.183 | −22.540 | 1.00 | 30.82 | B | C |
| ATOM | 3785 | CE | LYS | B | 197 | 4.561 | −10.244 | −22.886 | 1.00 | 33.46 | B | C |
| ATOM | 3786 | NZ | LYS | B | 197 | 5.890 | −9.721 | −23.354 | 1.00 | 34.43 | B | N |
| ATOM | 3787 | C | LYS | B | 197 | 3.174 | −10.537 | −17.792 | 1.00 | 23.41 | B | C |
| ATOM | 3788 | O | LYS | B | 197 | 4.038 | −11.381 | −17.745 | 1.00 | 23.66 | B | O |
| ATOM | 3789 | N | LEU | B | 198 | 2.975 | −9.712 | −16.779 | 1.00 | 22.98 | B | N |
| ATOM | 3790 | CA | LEU | B | 198 | 3.839 | −9.773 | −15.594 | 1.00 | 23.36 | B | C |
| ATOM | 3791 | CB | LEU | B | 198 | 3.497 | −8.658 | −14.588 | 1.00 | 24.55 | B | C |
| ATOM | 3792 | CG | LEU | B | 198 | 4.462 | −8.316 | −13.434 | 1.00 | 23.95 | B | C |
| ATOM | 3793 | CD1 | LEU | B | 198 | 5.905 | −8.032 | −13.877 | 1.00 | 22.89 | B | C |
| ATOM | 3794 | CD2 | LEU | B | 198 | 3.851 | −7.126 | −12.692 | 1.00 | 23.13 | B | C |
| ATOM | 3795 | C | LEU | B | 198 | 3.763 | −11.120 | −14.903 | 1.00 | 22.24 | B | C |
| ATOM | 3796 | O | LEU | B | 198 | 4.739 | −11.550 | −14.277 | 1.00 | 20.67 | B | O |
| ATOM | 3797 | N | TYR | B | 199 | 2.609 | −11.773 | −15.013 | 1.00 | 22.06 | B | N |
| ATOM | 3798 | CA | TYR | B | 199 | 2.432 | −13.111 | −14.444 | 1.00 | 22.94 | B | C |
| ATOM | 3799 | CB | TYR | B | 199 | 1.500 | −13.048 | −13.207 | 1.00 | 23.41 | B | C |
| ATOM | 3800 | CG | TYR | B | 199 | 1.901 | −11.985 | −12.194 | 1.00 | 23.95 | B | C |
| ATOM | 3801 | CD1 | TYR | B | 199 | 3.023 | −12.148 | −11.381 | 1.00 | 24.19 | B | C |
| ATOM | 3802 | CE1 | TYR | B | 199 | 3.400 | −11.169 | −10.474 | 1.00 | 24.03 | B | C |
| ATOM | 3803 | CZ | TYR | B | 199 | 2.647 | −10.011 | −10.355 | 1.00 | 24.37 | B | C |
| ATOM | 3804 | OH | TYR | B | 199 | 3.002 | −9.038 | −9.442 | 1.00 | 24.91 | B | O |
| ATOM | 3805 | CE2 | TYR | B | 199 | 1.543 | −9.819 | −11.152 | 1.00 | 24.38 | B | C |
| ATOM | 3806 | CD2 | TYR | B | 199 | 1.181 | −10.798 | −12.074 | 1.00 | 24.50 | B | C |
| ATOM | 3807 | C | TYR | B | 199 | 1.975 | −14.168 | −15.491 | 1.00 | 22.26 | B | C |
| ATOM | 3808 | O | TYR | B | 199 | 1.020 | −14.929 | −15.276 | 1.00 | 21.71 | B | O |
| ATOM | 3809 | N | ASN | B | 200 | 2.689 | −14.198 | −16.614 | 1.00 | 21.60 | B | N |
| ATOM | 3810 | CA | ASN | B | 200 | 2.589 | −15.264 | −17.620 | 1.00 | 21.13 | B | C |
| ATOM | 3811 | CB | ASN | B | 200 | 3.209 | −16.549 | −17.072 | 1.00 | 22.32 | B | C |
| ATOM | 3812 | CG | ASN | B | 200 | 4.606 | −16.323 | −16.485 | 1.00 | 23.87 | B | C |
| ATOM | 3813 | OD1 | ASN | B | 200 | 5.610 | −16.490 | −17.183 | 1.00 | 24.05 | B | O |
| ATOM | 3814 | ND2 | ASN | B | 200 | 4.675 | −15.933 | −15.190 | 1.00 | 24.03 | B | N |
| ATOM | 3815 | C | ASN | B | 200 | 1.163 | −15.477 | −18.168 | 1.00 | 19.76 | B | C |
| ATOM | 3816 | O | ASN | B | 200 | 0.696 | −16.607 | −18.388 | 1.00 | 17.76 | B | O |
| ATOM | 3817 | N | GLN | B | 201 | 0.495 | −14.348 | −18.381 | 1.00 | 18.92 | B | N |
| ATOM | 3818 | CA | GLN | B | 201 | −0.770 | −14.294 | −19.088 | 1.00 | 19.24 | B | C |
| ATOM | 3819 | CB | GLN | B | 201 | −1.840 | −13.585 | −18.250 | 1.00 | 17.82 | B | C |
| ATOM | 3820 | CG | GLN | B | 201 | −2.373 | −14.366 | −17.083 | 1.00 | 16.72 | B | C |
| ATOM | 3821 | CD | GLN | B | 201 | −3.552 | −13.685 | −16.459 | 1.00 | 16.42 | B | C |
| ATOM | 3822 | OE1 | GLN | B | 201 | −3.450 | −12.553 | −16.004 | 1.00 | 15.89 | B | O |
| ATOM | 3823 | NE2 | GLN | B | 201 | −4.687 | −14.371 | −16.429 | 1.00 | 16.72 | B | N |
| ATOM | 3824 | C | GLN | B | 201 | −0.600 | −13.490 | −20.369 | 1.00 | 20.79 | B | C |
| ATOM | 3825 | O | GLN | B | 201 | −0.009 | −12.417 | −20.355 | 1.00 | 22.47 | B | O |
| ATOM | 3826 | N | PRO | B | 202 | −1.160 | −13.970 | −21.471 | 1.00 | 21.98 | B | N |
| ATOM | 3827 | CA | PRO | B | 202 | −1.091 | −13.132 | −22.622 | 1.00 | 22.64 | B | C |
| ATOM | 3828 | CB | PRO | B | 202 | −1.481 | −14.079 | −23.742 | 1.00 | 22.77 | B | C |
| ATOM | 3829 | CG | PRO | B | 202 | −2.452 | −15.019 | −23.119 | 1.00 | 22.83 | B | C |
| ATOM | 3830 | CD | PRO | B | 202 | −2.109 | −15.080 | −21.656 | 1.00 | 22.57 | B | C |
| ATOM | 3831 | C | PRO | B | 202 | −2.085 | −11.965 | −22.505 | 1.00 | 24.85 | B | C |
| ATOM | 3832 | O | PRO | B | 202 | −3.127 | −12.061 | −21.810 | 1.00 | 23.59 | B | O |
| ATOM | 3833 | N | ASP | B | 203 | −1.737 | −10.880 | −23.202 | 1.00 | 27.61 | B | N |
| ATOM | 3834 | CA | ASP | B | 203 | −2.492 | −9.640 | −23.199 | 1.00 | 30.45 | B | C |
| ATOM | 3835 | CB | ASP | B | 203 | −1.610 | −8.492 | −22.682 | 1.00 | 32.67 | B | C |
| ATOM | 3836 | CG | ASP | B | 203 | −0.356 | −8.294 | −23.502 | 1.00 | 34.23 | B | C |
| ATOM | 3837 | OD1 | ASP | B | 203 | −0.252 | −8.865 | −24.613 | 1.00 | 36.10 | B | O |
| ATOM | 3838 | OD2 | ASP | B | 203 | 0.534 | −7.576 | −23.017 | 1.00 | 35.07 | B | O |
| ATOM | 3839 | C | ASP | B | 203 | −3.058 | −9.284 | −24.575 | 1.00 | 32.33 | B | C |
| ATOM | 3840 | O | ASP | B | 203 | −3.488 | −8.154 | −24.772 | 1.00 | 32.63 | B | O |
| ATOM | 3841 | N | ARG | B | 204 | −3.032 | −10.235 | −25.518 | 1.00 | 34.07 | B | N |
| ATOM | 3842 | CA | ARG | B | 204 | −3.744 | −10.117 | −26.791 | 1.00 | 34.55 | B | C |
| ATOM | 3843 | CB | ARG | B | 204 | −2.943 | −9.280 | −27.777 | 1.00 | 36.55 | B | C |
| ATOM | 3844 | CG | ARG | B | 204 | −1.516 | −9.767 | −27.975 | 1.00 | 39.06 | B | C |
| ATOM | 3845 | CD | ARG | B | 204 | −0.877 | −9.151 | −29.214 | 1.00 | 42.75 | B | C |
| ATOM | 3846 | NE | ARG | B | 204 | 0.412 | −9.771 | −29.545 | 1.00 | 49.37 | B | N |
| ATOM | 3847 | CZ | ARG | B | 204 | 1.388 | −9.199 | −30.267 | 1.00 | 53.04 | B | C |
| ATOM | 3848 | NH1 | ARG | B | 204 | 1.253 | −7.971 | −30.769 | 1.00 | 51.45 | B | N |
| ATOM | 3849 | NH2 | ARG | B | 204 | 2.525 | −9.858 | −30.482 | 1.00 | 53.50 | B | N |
| ATOM | 3850 | C | ARG | B | 204 | −4.071 | −11.495 | −27.408 | 1.00 | 36.34 | B | C |
| ATOM | 3851 | O | ARG | B | 204 | −3.251 | −12.429 | −27.360 | 1.00 | 36.07 | B | O |
| ATOM | 3852 | N | TYR | B | 205 | −5.271 | −11.592 | −28.001 | 1.00 | 36.40 | B | N |
| ATOM | 3853 | CA | TYR | B | 205 | −5.844 | −12.829 | −28.581 | 1.00 | 34.16 | B | C |
| ATOM | 3854 | CB | TYR | B | 205 | −7.022 | −12.465 | −29.497 | 1.00 | 34.62 | B | C |
| ATOM | 3855 | CG | TYR | B | 205 | −7.925 | −13.612 | −29.902 | 1.00 | 32.64 | B | C |
| ATOM | 3856 | CD1 | TYR | B | 205 | −8.842 | −14.136 | −29.001 | 1.00 | 32.71 | B | C |
| ATOM | 3857 | CE1 | TYR | B | 205 | −9.685 | −15.166 | −29.348 | 1.00 | 32.40 | B | C |
| ATOM | 3858 | CZ | TYR | B | 205 | −9.637 | −15.671 | −30.619 | 1.00 | 32.09 | B | C |
| ATOM | 3859 | OH | TYR | B | 205 | −10.483 | −16.696 | −30.926 | 1.00 | 29.46 | B | O |
| ATOM | 3860 | CE2 | TYR | B | 205 | −8.743 | −15.161 | −31.546 | 1.00 | 32.02 | B | C |
| ATOM | 3861 | CD2 | TYR | B | 205 | −7.898 | −14.132 | −31.185 | 1.00 | 31.56 | B | C |
| ATOM | 3862 | C | TYR | B | 205 | −4.872 | −13.655 | −29.394 | 1.00 | 32.77 | B | C |
| ATOM | 3863 | O | TYR | B | 205 | −4.928 | −14.872 | −29.361 | 1.00 | 31.27 | B | O |
| ATOM | 3864 | N | GLN | B | 206 | −3.996 | −12.993 | −30.136 | 1.00 | 32.81 | B | N |
| ATOM | 3865 | CA | GLN | B | 206 | −3.030 | −13.715 | −30.972 | 1.00 | 35.21 | B | C |
| ATOM | 3866 | CB | GLN | B | 206 | −2.272 | −12.841 | −32.044 | 1.00 | 38.64 | B | C |
| ATOM | 3867 | CG | GLN | B | 206 | −2.106 | −11.308 | −31.866 | 1.00 | 42.08 | B | C |
| ATOM | 3868 | CD | GLN | B | 206 | −3.425 | −10.488 | −31.920 | 1.00 | 43.10 | B | C |
| ATOM | 3869 | OE1 | GLN | B | 206 | −3.551 | −9.459 | −31.249 | 1.00 | 44.46 | B | O |
| ATOM | 3870 | NE2 | GLN | B | 206 | −4.410 | −10.960 | −32.688 | 1.00 | 42.28 | B | N |
| ATOM | 3871 | C | GLN | B | 206 | −2.071 | −14.536 | −30.129 | 1.00 | 33.13 | B | C |
| ATOM | 3872 | O | GLN | B | 206 | −1.563 | −15.534 | −30.607 | 1.00 | 35.03 | B | O |
| ATOM | 3873 | N | ASP | B | 207 | −1.855 | −14.166 | −28.870 | 1.00 | 31.41 | B | N |
| ATOM | 3874 | CA | ASP | B | 207 | −0.906 | −14.913 | −28.018 | 1.00 | 29.64 | B | C |
| ATOM | 3875 | CB | ASP | B | 207 | −0.053 | −13.937 | −27.197 | 1.00 | 29.62 | B | C |
| ATOM | 3876 | CG | ASP | B | 207 | 0.970 | −13.202 | −28.048 | 1.00 | 30.75 | B | C |
| ATOM | 3877 | OD1 | ASP | B | 207 | 1.183 | −13.628 | −29.212 | 1.00 | 30.64 | B | O |
| ATOM | 3878 | OD2 | ASP | B | 207 | 1.553 | −12.200 | −27.561 | 1.00 | 30.25 | B | O |
| ATOM | 3879 | C | ASP | B | 207 | −1.556 | −15.951 | −27.101 | 1.00 | 27.81 | B | C |
| ATOM | 3880 | O | ASP | B | 207 | −0.896 | −16.513 | −26.227 | 1.00 | 26.27 | B | O |
| ATOM | 3881 | N | ARG | B | 208 | −2.836 | −16.246 | −27.312 | 1.00 | 26.49 | B | N |
| ATOM | 3882 | CA | ARG | B | 208 | −3.536 | −17.097 | −26.368 | 1.00 | 24.72 | B | C |
| ATOM | 3883 | CB | ARG | B | 208 | −5.010 | −17.243 | −26.708 | 1.00 | 24.36 | B | C |
| ATOM | 3884 | CG | ARG | B | 208 | −5.326 | −17.924 | −28.024 | 1.00 | 24.48 | B | C |
| ATOM | 3885 | CD | ARG | B | 208 | −6.755 | −17.538 | −28.400 | 1.00 | 25.04 | B | C |
| ATOM | 3886 | NE | ARG | B | 208 | −7.264 | −18.154 | −29.617 | 1.00 | 24.76 | B | N |
| ATOM | 3887 | CZ | ARG | B | 208 | −6.783 | −17.933 | −30.837 | 1.00 | 25.11 | B | C |
| ATOM | 3888 | NH1 | ARG | B | 208 | −5.750 | −17.136 | −31.060 | 1.00 | 25.82 | B | N |
| ATOM | 3889 | NH2 | ARG | B | 208 | −7.337 | −18.534 | −31.858 | 1.00 | 26.12 | B | N |
| ATOM | 3890 | C | ARG | B | 208 | −2.920 | −18.451 | −26.283 | 1.00 | 24.48 | B | C |
| ATOM | 3891 | O | ARG | B | 208 | −2.232 | −18.908 | −27.185 | 1.00 | 25.71 | B | O |
| ATOM | 3892 | N | TYR | B | 209 | −3.188 | −19.103 | −25.167 | 1.00 | 24.84 | B | N |
| ATOM | 3893 | CA | TYR | B | 209 | −2.840 | −20.510 | −25.000 | 1.00 | 22.96 | B | C |
| ATOM | 3894 | CB | TYR | B | 209 | −2.724 | −20.842 | −23.521 | 1.00 | 21.57 | B | C |
| ATOM | 3895 | CG | TYR | B | 209 | −2.520 | −22.291 | −23.180 | 1.00 | 19.77 | B | C |
| ATOM | 3896 | CD1 | TYR | B | 209 | −1.260 | −22.868 | −23.218 | 1.00 | 18.75 | B | C |
| ATOM | 3897 | CE1 | TYR | B | 209 | −1.091 | −24.213 | −22.858 | 1.00 | 18.78 | B | C |
| ATOM | 3898 | CZ | TYR | B | 209 | −2.192 | −24.978 | −22.441 | 1.00 | 18.01 | B | C |
| ATOM | 3899 | OH | TYR | B | 209 | −2.057 | −26.288 | −22.082 | 1.00 | 17.53 | B | O |
| ATOM | 3900 | CE2 | TYR | B | 209 | −3.442 | −24.416 | −22.391 | 1.00 | 18.24 | B | C |
| ATOM | 3901 | CD2 | TYR | B | 209 | −3.603 | −23.077 | −22.740 | 1.00 | 19.13 | B | C |
| ATOM | 3902 | C | TYR | B | 209 | −3.912 | −21.351 | −25.673 | 1.00 | 22.56 | B | C |
| ATOM | 3903 | O | TYR | B | 209 | −5.106 | −21.048 | −25.608 | 1.00 | 21.55 | B | O |
| ATOM | 3904 | N | THR | B | 210 | −3.438 | −22.421 | −26.304 | 1.00 | 23.19 | B | N |
| ATOM | 3905 | CA | THR | B | 210 | −4.190 | −23.254 | −27.246 | 1.00 | 21.30 | B | C |
| ATOM | 3906 | CB | THR | B | 210 | −3.509 | −23.070 | −28.627 | 1.00 | 20.51 | B | C |
| ATOM | 3907 | OG1 | THR | B | 210 | −4.283 | −22.128 | −29.351 | 1.00 | 19.96 | B | O |
| ATOM | 3908 | CG2 | THR | B | 210 | −3.380 | −24.329 | −29.449 | 1.00 | 21.23 | B | C |
| ATOM | 3909 | C | THR | B | 210 | −4.291 | −24.717 | −26.770 | 1.00 | 20.49 | B | C |
| ATOM | 3910 | O | THR | B | 210 | −4.989 | −25.528 | −27.385 | 1.00 | 22.84 | B | O |
| ATOM | 3911 | N | GLY | B | 211 | −3.622 | −25.037 | −25.662 | 1.00 | 18.65 | B | N |
| ATOM | 3912 | CA | GLY | B | 211 | −3.502 | −26.404 | −25.164 | 1.00 | 17.86 | B | C |
| ATOM | 3913 | C | GLY | B | 211 | −4.688 | −26.812 | −24.313 | 1.00 | 17.46 | B | C |
| ATOM | 3914 | O | GLY | B | 211 | −5.628 | −26.022 | −24.134 | 1.00 | 17.51 | B | O |
| ATOM | 3915 | N | PRO | B | 212 | −4.664 | −28.052 | −23.801 | 1.00 | 16.80 | B | N |
| ATOM | 3916 | CA | PRO | B | 212 | −5.780 | −28.673 | −23.088 | 1.00 | 17.36 | B | C |
| ATOM | 3917 | CB | PRO | B | 212 | −5.594 | −30.156 | −23.396 | 1.00 | 16.89 | B | C |
| ATOM | 3918 | CG | PRO | B | 212 | −4.113 | −30.308 | −23.446 | 1.00 | 17.16 | B | C |
| ATOM | 3919 | CD | PRO | B | 212 | −3.604 | −29.030 | −24.083 | 1.00 | 17.00 | B | C |
| ATOM | 3920 | C | PRO | B | 212 | −5.769 | −28.494 | −21.573 | 1.00 | 18.49 | B | C |
| ATOM | 3921 | O | PRO | B | 212 | −6.773 | −28.798 | −20.908 | 1.00 | 19.01 | B | O |
| ATOM | 3922 | N | SER | B | 213 | −4.645 | −28.019 | −21.056 | 1.00 | 18.86 | B | N |
| ATOM | 3923 | CA | SER | B | 213 | −4.391 | −27.910 | −19.645 | 1.00 | 19.95 | B | C |
| ATOM | 3924 | CB | SER | B | 213 | −2.881 | −27.698 | −19.495 | 1.00 | 22.65 | B | C |
| ATOM | 3925 | OG | SER | B | 213 | −2.471 | −27.825 | −18.161 | 1.00 | 26.25 | B | O |
| ATOM | 3926 | C | SER | B | 213 | −5.168 | −26.724 | −19.078 | 1.00 | 18.23 | B | C |
| ATOM | 3927 | O | SER | B | 213 | −5.345 | −25.770 | −19.779 | 1.00 | 16.91 | B | O |
| ATOM | 3928 | N | ASP | B | 214 | −5.635 | −26.785 | −17.824 | 1.00 | 18.11 | B | N |
| ATOM | 3929 | CA | ASP | B | 214 | −6.440 | −25.689 | −17.227 | 1.00 | 17.78 | B | C |
| ATOM | 3930 | CB | ASP | B | 214 | −5.596 | −24.409 | −17.164 | 1.00 | 17.90 | B | C |
| ATOM | 3931 | CG | ASP | B | 214 | −6.244 | −23.295 | −16.378 | 1.00 | 17.76 | B | C |
| ATOM | 3932 | OD1 | ASP | B | 214 | −7.112 | −23.546 | −15.494 | 1.00 | 18.10 | B | O |
| ATOM | 3933 | OD2 | ASP | B | 214 | −5.857 | −22.141 | −16.651 | 1.00 | 17.17 | B | O |
| ATOM | 3934 | C | ASP | B | 214 | −7.708 | −25.443 | −18.049 | 1.00 | 18.13 | B | C |
| ATOM | 3935 | O | ASP | B | 214 | −7.986 | −24.311 | −18.477 | 1.00 | 16.78 | B | O |
| ATOM | 3936 | N | ASN | B | 215 | −8.446 | −26.527 | −18.296 | 1.00 | 18.96 | B | N |
| ATOM | 3937 | CA | ASN | B | 215 | −9.601 | −26.515 | −19.196 | 1.00 | 19.63 | B | C |
| ATOM | 3938 | CB | ASN | B | 215 | −10.848 | −26.092 | −18.422 | 1.00 | 20.32 | B | C |
| ATOM | 3939 | CG | ASN | B | 215 | −11.257 | −27.138 | −17.405 | 1.00 | 20.81 | B | C |
| ATOM | 3940 | OD1 | ASN | B | 215 | −11.299 | −28.333 | −17.713 | 1.00 | 21.24 | B | O |
| ATOM | 3941 | ND2 | ASN | B | 215 | −11.492 | −26.715 | −16.188 | 1.00 | 21.02 | B | N |
| ATOM | 3942 | C | ASN | B | 215 | −9.354 | −25.645 | −20.420 | 1.00 | 20.01 | B | C |
| ATOM | 3943 | O | ASN | B | 215 | −10.137 | −24.759 | −20.766 | 1.00 | 20.66 | B | O |
| ATOM | 3944 | N | GLY | B | 216 | −8.219 | −25.896 | −21.058 | 1.00 | 20.01 | B | N |
| ATOM | 3945 | CA | GLY | B | 216 | −7.796 | −25.146 | −22.226 | 1.00 | 19.24 | B | C |
| ATOM | 3946 | C | GLY | B | 216 | −7.633 | −23.662 | −21.985 | 1.00 | 18.69 | B | C |
| ATOM | 3947 | O | GLY | B | 216 | −8.297 | −22.859 | −22.638 | 1.00 | 18.64 | B | O |
| ATOM | 3948 | N | GLY | B | 217 | −6.775 | −23.303 | −21.030 | 1.00 | 18.39 | B | N |
| ATOM | 3949 | CA | GLY | B | 217 | −6.297 | −21.910 | −20.845 | 1.00 | 18.17 | B | C |
| ATOM | 3950 | C | GLY | B | 217 | −7.079 | −20.998 | −19.908 | 1.00 | 17.90 | B | C |
| ATOM | 3951 | O | GLY | B | 217 | −6.747 | −19.836 | −19.739 | 1.00 | 18.36 | B | O |
| ATOM | 3952 | N | VAL | B | 218 | −8.082 | −21.542 | −19.248 | 1.00 | 18.00 | B | N |
| ATOM | 3953 | CA | VAL | B | 218 | −9.075 | −20.757 | −18.535 | 1.00 | 18.49 | B | C |
| ATOM | 3954 | CB | VAL | B | 218 | −9.978 | −21.757 | −17.786 | 1.00 | 18.67 | B | C |
| ATOM | 3955 | CG1 | VAL | B | 218 | −10.669 | −21.159 | −16.609 | 1.00 | 19.04 | B | C |
| ATOM | 3956 | CG2 | VAL | B | 218 | −11.017 | −22.288 | −18.752 | 1.00 | 19.24 | B | C |
| ATOM | 3957 | C | VAL | B | 218 | −8.520 | −19.593 | −17.660 | 1.00 | 19.13 | B | C |
| ATOM | 3958 | O | VAL | B | 218 | −9.025 | −18.488 | −17.723 | 1.00 | 18.61 | B | O |
| ATOM | 3959 | N | HIS | B | 219 | −7.480 | −19.833 | −16.865 | 1.00 | 19.66 | B | N |
| ATOM | 3960 | CA | HIS | B | 219 | −6.885 | −18.786 | −16.037 | 1.00 | 19.59 | B | C |
| ATOM | 3961 | CB | HIS | B | 219 | −6.449 | −19.301 | −14.641 | 1.00 | 20.15 | B | C |
| ATOM | 3962 | CG | HIS | B | 219 | −7.529 | −19.983 | −13.848 | 1.00 | 19.47 | B | C |
| ATOM | 3963 | ND1 | HIS | B | 219 | −7.816 | −21.327 | −13.979 | 1.00 | 19.54 | B | N |
| ATOM | 3964 | CE1 | HIS | B | 219 | −8.807 | −21.643 | −13.163 | 1.00 | 19.59 | B | C |
| ATOM | 3965 | NE2 | HIS | B | 219 | −9.158 | −20.559 | −12.494 | 1.00 | 18.98 | B | N |
| ATOM | 3966 | CD2 | HIS | B | 219 | −8.361 | −19.513 | −12.891 | 1.00 | 19.03 | B | C |
| ATOM | 3967 | C | HIS | B | 219 | −5.658 | −18.243 | −16.725 | 1.00 | 20.25 | B | C |
| ATOM | 3968 | O | HIS | B | 219 | −5.105 | −17.241 | −16.283 | 1.00 | 20.29 | B | O |
| ATOM | 3969 | N | ILE | B | 220 | −5.171 | −18.915 | −17.766 | 1.00 | 21.20 | B | N |
| ATOM | 3970 | CA | ILE | B | 220 | −4.097 | −18.309 | −18.566 | 1.00 | 21.97 | B | C |
| ATOM | 3971 | CB | ILE | B | 220 | −3.375 | −19.323 | −19.457 | 1.00 | 22.68 | B | C |
| ATOM | 3972 | CG1 | ILE | B | 220 | −2.724 | −20.393 | −18.595 | 1.00 | 23.99 | B | C |
| ATOM | 3973 | CD1 | ILE | B | 220 | −2.178 | −21.553 | −19.404 | 1.00 | 24.68 | B | C |
| ATOM | 3974 | CG2 | ILE | B | 220 | −2.293 | −18.655 | −20.293 | 1.00 | 22.37 | B | C |
| ATOM | 3975 | C | ILE | B | 220 | −4.698 | −17.207 | −19.426 | 1.00 | 22.02 | B | C |
| ATOM | 3976 | O | ILE | B | 220 | −4.261 | −16.068 | −19.344 | 1.00 | 23.21 | B | O |
| ATOM | 3977 | N | ASN | B | 221 | −5.724 | −17.545 | −20.209 | 1.00 | 20.75 | B | N |
| ATOM | 3978 | CA | ASN | B | 221 | −6.311 | −16.624 | −21.182 | 1.00 | 20.24 | B | C |
| ATOM | 3979 | CB | ASN | B | 221 | −6.996 | −17.406 | −22.300 | 1.00 | 18.70 | B | C |
| ATOM | 3980 | CG | ASN | B | 221 | −6.009 | −18.178 | −23.149 | 1.00 | 17.72 | B | C |
| ATOM | 3981 | OD1 | ASN | B | 221 | −4.883 | −17.752 | −23.379 | 1.00 | 17.36 | B | O |
| ATOM | 3982 | ND2 | ASN | B | 221 | −6.425 | −19.321 | −23.609 | 1.00 | 16.99 | B | N |
| ATOM | 3983 | C | ASN | B | 221 | −7.291 | −15.582 | −20.631 | 1.00 | 21.78 | B | C |
| ATOM | 3984 | O | ASN | B | 221 | −7.719 | −14.705 | −21.386 | 1.00 | 21.93 | B | O |
| ATOM | 3985 | N | SER | B | 222 | −7.651 | −15.633 | −19.346 | 1.00 | 22.00 | B | N |
| ATOM | 3986 | CA | SER | B | 222 | −8.578 | −14.633 | −18.860 | 1.00 | 22.02 | B | C |
| ATOM | 3987 | CB | SER | B | 222 | −9.109 | −14.959 | −17.466 | 1.00 | 22.44 | B | C |
| ATOM | 3988 | OG | SER | B | 222 | −8.090 | −15.068 | −16.478 | 1.00 | 23.22 | B | O |
| ATOM | 3989 | C | SER | B | 222 | −7.852 | −13.297 | −18.931 | 1.00 | 23.31 | B | C |
| ATOM | 3990 | O | SER | B | 222 | −8.481 | −12.230 | −19.024 | 1.00 | 22.58 | B | O |
| ATOM | 3991 | N | GLY | B | 223 | −6.516 | −13.371 | −18.945 | 1.00 | 24.52 | B | N |
| ATOM | 3992 | CA | GLY | B | 223 | −5.651 | −12.186 | −19.052 | 1.00 | 25.84 | B | C |
| ATOM | 3993 | C | GLY | B | 223 | −6.008 | −11.231 | −20.177 | 1.00 | 26.96 | B | C |
| ATOM | 3994 | O | GLY | B | 223 | −5.884 | −10.001 | −20.034 | 1.00 | 28.19 | B | O |
| ATOM | 3995 | N | ILE | B | 224 | −6.437 | −11.809 | −21.296 | 1.00 | 26.49 | B | N |
| ATOM | 3996 | CA | ILE | B | 224 | −6.923 | −11.057 | −22.459 | 1.00 | 25.44 | B | C |
| ATOM | 3997 | CB | ILE | B | 224 | −7.187 | −12.011 | −23.637 | 1.00 | 24.70 | B | C |
| ATOM | 3998 | CG1 | ILE | B | 224 | −5.849 | −12.487 | −24.198 | 1.00 | 24.83 | B | C |
| ATOM | 3999 | CD1 | ILE | B | 224 | −5.956 | −13.782 | −24.968 | 1.00 | 25.47 | B | C |
| ATOM | 4000 | CG2 | ILE | B | 224 | −8.002 | −11.327 | −24.720 | 1.00 | 24.67 | B | C |
| ATOM | 4001 | C | ILE | B | 224 | −8.172 | −10.209 | −22.179 | 1.00 | 24.64 | B | C |
| ATOM | 4002 | O | ILE | B | 224 | −8.209 | −9.029 | −22.468 | 1.00 | 25.66 | B | O |
| ATOM | 4003 | N | ASN | B | 225 | −9.212 | −10.798 | −21.637 | 1.00 | 24.78 | B | N |
| ATOM | 4004 | CA | ASN | B | 225 | −10.377 | −10.011 | −21.335 | 1.00 | 24.71 | B | C |
| ATOM | 4005 | CB | ASN | B | 225 | −11.549 | −10.903 | −20.978 | 1.00 | 24.55 | B | C |
| ATOM | 4006 | CG | ASN | B | 225 | −12.869 | −10.188 | −21.126 | 1.00 | 24.56 | B | C |
| ATOM | 4007 | OD1 | ASN | B | 225 | −13.625 | −9.979 | −20.164 | 1.00 | 23.26 | B | O |
| ATOM | 4008 | ND2 | ASN | B | 225 | −13.143 | −9.775 | −22.347 | 1.00 | 25.30 | B | N |
| ATOM | 4009 | C | ASN | B | 225 | −10.097 | −9.051 | −20.188 | 1.00 | 25.63 | B | C |
| ATOM | 4010 | O | ASN | B | 225 | −10.577 | −7.922 | −20.188 | 1.00 | 25.70 | B | O |
| ATOM | 4011 | N | ASN | B | 226 | −9.321 | −9.494 | −19.204 | 1.00 | 26.62 | B | N |
| ATOM | 4012 | CA | ASN | B | 226 | −8.969 | −8.608 | −18.104 | 1.00 | 26.81 | B | C |
| ATOM | 4013 | CB | ASN | B | 226 | −8.135 | −9.330 | −17.035 | 1.00 | 27.03 | B | C |
| ATOM | 4014 | CG | ASN | B | 226 | −8.963 | −10.256 | −16.156 | 1.00 | 27.23 | B | C |
| ATOM | 4015 | OD1 | ASN | B | 226 | −10.195 | −10.313 | −16.254 | 1.00 | 26.96 | B | O |
| ATOM | 4016 | ND2 | ASN | B | 226 | −8.277 | −11.001 | −15.289 | 1.00 | 27.06 | B | N |
| ATOM | 4017 | C | ASN | B | 226 | −8.243 | −7.340 | −18.594 | 1.00 | 26.48 | B | C |
| ATOM | 4018 | O | ASN | B | 226 | −8.512 | −6.254 | −18.096 | 1.00 | 28.43 | B | O |
| ATOM | 4019 | N | LYS | B | 227 | −7.343 | −7.458 | −19.558 | 1.00 | 24.83 | B | N |
| ATOM | 4020 | CA | LYS | B | 227 | −6.685 | −6.271 | −20.081 | 1.00 | 25.45 | B | C |
| ATOM | 4021 | CB | LYS | B | 227 | −5.631 | −6.652 | −21.096 | 1.00 | 25.83 | B | C |
| ATOM | 4022 | CG | LYS | B | 227 | −4.834 | −5.495 | −21.646 | 1.00 | 25.86 | B | C |
| ATOM | 4023 | CD | LYS | B | 227 | −3.856 | −4.952 | −20.632 | 1.00 | 27.34 | B | C |
| ATOM | 4024 | CE | LYS | B | 227 | −3.290 | −3.619 | −21.118 | 1.00 | 28.38 | B | C |
| ATOM | 4025 | NZ | LYS | B | 227 | −2.147 | −3.154 | −20.291 | 1.00 | 28.32 | B | N |
| ATOM | 4026 | C | LYS | B | 227 | −7.675 | −5.333 | −20.742 | 1.00 | 26.25 | B | C |
| ATOM | 4027 | O | LYS | B | 227 | −7.581 | −4.130 | −20.580 | 1.00 | 27.67 | B | O |
| ATOM | 4028 | N | ALA | B | 228 | −8.605 | −5.884 | −21.509 | 1.00 | 26.38 | B | N |
| ATOM | 4029 | CA | ALA | B | 228 | −9.669 | −5.091 | −22.101 | 1.00 | 26.22 | B | C |
| ATOM | 4030 | CB | ALA | B | 228 | −10.670 | −5.974 | −22.840 | 1.00 | 26.76 | B | C |
| ATOM | 4031 | C | ALA | B | 228 | −10.387 | −4.320 | −21.027 | 1.00 | 26.05 | B | C |
| ATOM | 4032 | O | ALA | B | 228 | −10.645 | −3.149 | −21.190 | 1.00 | 28.08 | B | O |
| ATOM | 4033 | N | PHE | B | 229 | −10.725 | −4.969 | −19.926 | 1.00 | 25.85 | B | N |
| ATOM | 4034 | CA | PHE | B | 229 | −11.412 | −4.266 | −18.853 | 1.00 | 25.93 | B | C |
| ATOM | 4035 | CB | PHE | B | 229 | −11.755 | −5.203 | −17.712 | 1.00 | 25.94 | B | C |
| ATOM | 4036 | CG | PHE | B | 229 | −12.518 | −4.533 | −16.642 | 1.00 | 26.06 | B | C |
| ATOM | 4037 | CD1 | PHE | B | 229 | −13.890 | −4.407 | −16.748 | 1.00 | 26.92 | B | C |
| ATOM | 4038 | CE1 | PHE | B | 229 | −14.620 | −3.758 | −15.766 | 1.00 | 27.95 | B | C |
| ATOM | 4039 | CZ | PHE | B | 229 | −13.963 | −3.228 | −14.661 | 1.00 | 27.83 | B | C |
| ATOM | 4040 | CE2 | PHE | B | 229 | −12.580 | −3.341 | −14.562 | 1.00 | 27.45 | B | C |
| ATOM | 4041 | CD2 | PHE | B | 229 | −11.868 | −3.985 | −15.551 | 1.00 | 26.96 | B | C |
| ATOM | 4042 | C | PHE | B | 229 | −10.626 | −3.063 | −18.313 | 1.00 | 25.50 | B | C |
| ATOM | 4043 | O | PHE | B | 229 | −11.162 | −1.968 | −18.214 | 1.00 | 26.44 | B | O |
| ATOM | 4044 | N | TYR | B | 230 | −9.362 | −3.262 | −17.970 | 1.00 | 25.44 | B | N |
| ATOM | 4045 | CA | TYR | B | 230 | −8.488 | −2.153 | −17.546 | 1.00 | 26.60 | B | C |
| ATOM | 4046 | CB | TYR | B | 230 | −7.055 | −2.645 | −17.394 | 1.00 | 26.50 | B | C |
| ATOM | 4047 | CG | TYR | B | 230 | −6.057 | −1.545 | −17.152 | 1.00 | 26.33 | B | C |
| ATOM | 4048 | CD1 | TYR | B | 230 | −5.980 | −0.908 | −15.912 | 1.00 | 25.76 | B | C |
| ATOM | 4049 | CE1 | TYR | B | 230 | −5.055 | 0.087 | −15.667 | 1.00 | 25.25 | B | C |
| ATOM | 4050 | CZ | TYR | B | 230 | −4.191 | 0.458 | −16.664 | 1.00 | 26.43 | B | C |
| ATOM | 4051 | OH | TYR | B | 230 | −3.275 | 1.445 | −16.431 | 1.00 | 27.30 | B | O |
| ATOM | 4052 | CE2 | TYR | B | 230 | −4.237 | −0.160 | −17.903 | 1.00 | 27.11 | B | C |
| ATOM | 4053 | CD2 | TYR | B | 230 | −5.181 | −1.149 | −18.146 | 1.00 | 26.74 | B | C |
| ATOM | 4054 | C | TYR | B | 230 | −8.455 | −0.948 | −18.499 | 1.00 | 26.53 | B | C |
| ATOM | 4055 | O | TYR | B | 230 | −8.456 | 0.196 | −18.050 | 1.00 | 26.33 | B | O |
| ATOM | 4056 | N | LEU | B | 231 | −8.386 | −1.221 | −19.798 | 1.00 | 26.27 | B | N |
| ATOM | 4057 | CA | LEU | B | 231 | −8.373 | −0.180 | −20.809 | 1.00 | 26.57 | B | C |
| ATOM | 4058 | CB | LEU | B | 231 | −8.041 | −0.775 | −22.171 | 1.00 | 25.37 | B | C |
| ATOM | 4059 | CG | LEU | B | 231 | −6.568 | −1.208 | −22.262 | 1.00 | 25.50 | B | C |
| ATOM | 4060 | CD1 | LEU | B | 231 | −6.257 | −2.057 | −23.476 | 1.00 | 25.18 | B | C |
| ATOM | 4061 | CD2 | LEU | B | 231 | −5.657 | 0.007 | −22.268 | 1.00 | 26.56 | B | C |
| ATOM | 4062 | C | LEU | B | 231 | −9.705 | 0.574 | −20.817 | 1.00 | 28.83 | B | C |
| ATOM | 4063 | O | LEU | B | 231 | −9.743 | 1.805 | −20.856 | 1.00 | 30.14 | B | O |
| ATOM | 4064 | N | ILE | B | 232 | −10.799 | −0.156 | −20.705 | 1.00 | 29.54 | B | N |
| ATOM | 4065 | CA | ILE | B | 232 | −12.115 | 0.458 | −20.640 | 1.00 | 29.21 | B | C |
| ATOM | 4066 | CB | ILE | B | 232 | −13.197 | −0.634 | −20.677 | 1.00 | 28.64 | B | C |
| ATOM | 4067 | CG1 | ILE | B | 232 | −13.234 | −1.247 | −22.084 | 1.00 | 29.59 | B | C |
| ATOM | 4068 | CD1 | ILE | B | 232 | −14.053 | −2.517 | −22.206 | 1.00 | 29.92 | B | C |
| ATOM | 4069 | CG2 | ILE | B | 232 | −14.561 | −0.084 | −20.291 | 1.00 | 28.76 | B | C |
| ATOM | 4070 | C | ILE | B | 232 | −12.269 | 1.291 | −19.374 | 1.00 | 30.72 | B | C |
| ATOM | 4071 | O | ILE | B | 232 | −13.009 | 2.273 | −19.340 | 1.00 | 33.34 | B | O |
| ATOM | 4072 | N | ALA | B | 233 | −11.595 | 0.888 | −18.309 | 1.00 | 31.94 | B | N |
| ATOM | 4073 | CA | ALA | B | 233 | −11.820 | 1.522 | −17.007 | 1.00 | 30.90 | B | C |
| ATOM | 4074 | CB | ALA | B | 233 | −11.604 | 0.509 | −15.900 | 1.00 | 31.09 | B | C |
| ATOM | 4075 | C | ALA | B | 233 | −10.896 | 2.715 | −16.829 | 1.00 | 29.25 | B | C |
| ATOM | 4076 | O | ALA | B | 233 | −11.338 | 3.839 | −16.703 | 1.00 | 26.84 | B | O |
| ATOM | 4077 | N | GLN | B | 234 | −9.601 | 2.438 | −16.850 | 1.00 | 28.21 | B | N |
| ATOM | 4078 | CA | GLN | B | 234 | −8.605 | 3.442 | −16.643 | 1.00 | 27.52 | B | C |
| ATOM | 4079 | CB | GLN | B | 234 | −7.332 | 2.775 | −16.136 | 1.00 | 27.99 | B | C |
| ATOM | 4080 | CG | GLN | B | 234 | −6.297 | 3.757 | −15.629 | 1.00 | 27.70 | B | C |
| ATOM | 4081 | CD | GLN | B | 234 | −6.901 | 4.805 | −14.703 | 1.00 | 27.94 | B | C |
| ATOM | 4082 | OE1 | GLN | B | 234 | −7.897 | 4.556 | −13.997 | 1.00 | 27.32 | B | O |
| ATOM | 4083 | NE2 | GLN | B | 234 | −6.301 | 5.988 | −14.702 | 1.00 | 27.80 | B | N |
| ATOM | 4084 | C | GLN | B | 234 | −8.257 | 4.222 | −17.892 | 1.00 | 27.94 | B | C |
| ATOM | 4085 | O | GLN | B | 234 | −7.823 | 5.377 | −17.791 | 1.00 | 28.34 | B | O |
| ATOM | 4086 | N | GLY | B | 235 | −8.417 | 3.590 | −19.059 | 1.00 | 27.37 | B | N |
| ATOM | 4087 | CA | GLY | B | 235 | −7.817 | 4.077 | −20.296 | 1.00 | 25.77 | B | C |
| ATOM | 4088 | C | GLY | B | 235 | −6.345 | 3.708 | −20.328 | 1.00 | 25.72 | B | C |
| ATOM | 4089 | O | GLY | B | 235 | −5.740 | 3.433 | −19.292 | 1.00 | 25.36 | B | O |
| ATOM | 4090 | N | GLY | B | 236 | −5.769 | 3.700 | −21.523 | 1.00 | 25.94 | B | N |
| ATOM | 4091 | CA | GLY | B | 236 | −4.350 | 3.423 | −21.698 | 1.00 | 26.60 | B | C |
| ATOM | 4092 | C | GLY | B | 236 | −4.044 | 3.322 | −23.177 | 1.00 | 28.63 | B | C |
| ATOM | 4093 | O | GLY | B | 236 | −4.919 | 3.572 | −24.017 | 1.00 | 29.26 | B | O |
| ATOM | 4094 | N | THR | B | 237 | −2.801 | 2.970 | −23.485 | 1.00 | 29.75 | B | N |
| ATOM | 4095 | CA | THR | B | 237 | −2.327 | 2.831 | −24.842 | 1.00 | 31.58 | B | C |
| ATOM | 4096 | CB | THR | B | 237 | −1.327 | 3.962 | −25.206 | 1.00 | 33.98 | B | C |
| ATOM | 4097 | OG1 | THR | B | 237 | −2.063 | 5.104 | −25.633 | 1.00 | 37.98 | B | O |
| ATOM | 4098 | CG2 | THR | B | 237 | −0.360 | 3.594 | −26.356 | 1.00 | 34.58 | B | C |
| ATOM | 4099 | C | THR | B | 237 | −1.650 | 1.496 | −24.899 | 1.00 | 34.03 | B | C |
| ATOM | 4100 | O | THR | B | 237 | −0.632 | 1.298 | −24.230 | 1.00 | 34.87 | B | O |
| ATOM | 4101 | N | HIS | B | 238 | −2.184 | 0.602 | −25.731 | 1.00 | 36.70 | B | N |
| ATOM | 4102 | CA | HIS | B | 238 | −1.747 | −0.790 | −25.793 | 1.00 | 37.24 | B | C |
| ATOM | 4103 | CB | HIS | B | 238 | −2.711 | −1.577 | −24.898 | 1.00 | 42.00 | B | C |
| ATOM | 4104 | CG | HIS | B | 238 | −2.361 | −3.020 | −24.728 | 1.00 | 43.70 | B | C |
| ATOM | 4105 | ND1 | HIS | B | 238 | −3.268 | −4.030 | −24.968 | 1.00 | 43.48 | B | N |
| ATOM | 4106 | CE1 | HIS | B | 238 | −2.691 | −5.194 | −24.734 | 1.00 | 46.45 | B | C |
| ATOM | 4107 | NE2 | HIS | B | 238 | −1.445 | −4.974 | −24.351 | 1.00 | 47.60 | B | N |
| ATOM | 4108 | CD2 | HIS | B | 238 | −1.213 | −3.620 | −24.335 | 1.00 | 45.16 | B | C |
| ATOM | 4109 | C | HIS | B | 238 | −1.769 | −1.323 | −27.239 | 1.00 | 35.71 | B | C |
| ATOM | 4110 | O | HIS | B | 238 | −2.832 | −1.322 | −27.876 | 1.00 | 31.39 | B | O |
| ATOM | 4111 | N | TYR | B | 239 | −0.616 | −1.798 | −27.743 | 1.00 | 37.49 | B | N |
| ATOM | 4112 | CA | TYR | B | 239 | −0.431 | −2.163 | −29.201 | 1.00 | 39.68 | B | C |
| ATOM | 4113 | CB | TYR | B | 239 | −0.973 | −3.577 | −29.559 | 1.00 | 38.30 | B | C |
| ATOM | 4114 | CG | TYR | B | 239 | −0.280 | −4.661 | −28.775 | 1.00 | 36.05 | B | C |
| ATOM | 4115 | CD1 | TYR | B | 239 | 1.087 | −4.867 | −28.915 | 1.00 | 36.03 | B | C |
| ATOM | 4116 | CE1 | TYR | B | 239 | 1.755 | −5.830 | −28.174 | 1.00 | 36.29 | B | C |
| ATOM | 4117 | CZ | TYR | B | 239 | 1.042 | −6.605 | −27.259 | 1.00 | 37.19 | B | C |
| ATOM | 4118 | OH | TYR | B | 239 | 1.707 | −7.570 | −26.501 | 1.00 | 34.74 | B | O |
| ATOM | 4119 | CE2 | TYR | B | 239 | −0.333 | −6.403 | −27.104 | 1.00 | 36.29 | B | C |
| ATOM | 4120 | CD2 | TYR | B | 239 | −0.975 | −5.433 | −27.855 | 1.00 | 35.25 | B | C |
| ATOM | 4121 | C | TYR | B | 239 | −1.026 | −1.116 | −30.152 | 1.00 | 41.11 | B | C |
| ATOM | 4122 | O | TYR | B | 239 | −2.046 | −1.350 | −30.818 | 1.00 | 40.61 | B | O |
| ATOM | 4123 | N | GLY | B | 240 | −0.401 | 0.057 | −30.164 | 1.00 | 42.40 | B | N |
| ATOM | 4124 | CA | GLY | B | 240 | −0.862 | 1.165 | −30.985 | 1.00 | 43.82 | B | C |
| ATOM | 4125 | C | GLY | B | 240 | −2.136 | 1.887 | −30.549 | 1.00 | 42.06 | B | C |
| ATOM | 4126 | O | GLY | B | 240 | −2.231 | 3.116 | −30.685 | 1.00 | 42.31 | B | O |
| ATOM | 4127 | N | VAL | B | 241 | −3.121 | 1.152 | −30.047 | 1.00 | 38.05 | B | N |
| ATOM | 4128 | CA | VAL | B | 241 | −4.450 | 1.724 | −29.817 | 1.00 | 37.68 | B | C |
| ATOM | 4129 | CB | VAL | B | 241 | −5.488 | 0.599 | −29.856 | 1.00 | 35.77 | B | C |
| ATOM | 4130 | CG1 | VAL | B | 241 | −6.907 | 1.130 | −29.740 | 1.00 | 34.09 | B | C |
| ATOM | 4131 | CG2 | VAL | B | 241 | −5.277 | −0.194 | −31.139 | 1.00 | 35.98 | B | C |
| ATOM | 4132 | C | VAL | B | 241 | −4.524 | 2.586 | −28.519 | 1.00 | 38.81 | B | C |
| ATOM | 4133 | O | VAL | B | 241 | −3.917 | 2.253 | −27.499 | 1.00 | 39.08 | B | O |
| ATOM | 4134 | N | THR | B | 242 | −5.204 | 3.732 | −28.605 | 1.00 | 38.22 | B | N |
| ATOM | 4135 | CA | THR | B | 242 | −5.407 | 4.614 | −27.465 | 1.00 | 38.17 | B | C |
| ATOM | 4136 | CB | THR | B | 242 | −5.044 | 6.083 | −27.805 | 1.00 | 37.28 | B | C |
| ATOM | 4137 | OG1 | THR | B | 242 | −3.630 | 6.233 | −27.717 | 1.00 | 38.40 | B | O |
| ATOM | 4138 | CG2 | THR | B | 242 | −5.670 | 7.096 | −26.838 | 1.00 | 36.94 | B | C |
| ATOM | 4139 | C | THR | B | 242 | −6.865 | 4.458 | −27.034 | 1.00 | 39.27 | B | C |
| ATOM | 4140 | O | THR | B | 242 | −7.788 | 4.557 | −27.851 | 1.00 | 39.33 | B | O |
| ATOM | 4141 | N | VAL | B | 243 | −7.064 | 4.176 | −25.752 | 1.00 | 38.72 | B | N |
| ATOM | 4142 | CA | VAL | B | 243 | −8.396 | 4.046 | −25.213 | 1.00 | 38.15 | B | C |
| ATOM | 4143 | CB | VAL | B | 243 | −8.615 | 2.696 | −24.551 | 1.00 | 36.94 | B | C |
| ATOM | 4144 | CG1 | VAL | B | 243 | −10.085 | 2.522 | −24.211 | 1.00 | 37.69 | B | C |
| ATOM | 4145 | CG2 | VAL | B | 243 | −8.171 | 1.598 | −25.492 | 1.00 | 39.28 | B | C |
| ATOM | 4146 | C | VAL | B | 243 | −8.566 | 5.090 | −24.162 | 1.00 | 39.74 | B | C |
| ATOM | 4147 | O | VAL | B | 243 | −7.658 | 5.304 | −23.362 | 1.00 | 40.60 | B | O |
| ATOM | 4148 | N | ASN | B | 244 | −9.727 | 5.736 | −24.174 | 1.00 | 40.74 | B | N |
| ATOM | 4149 | CA | ASN | B | 244 | −10.041 | 6.753 | −23.201 | 1.00 | 40.96 | B | C |
| ATOM | 4150 | CB | ASN | B | 244 | −10.781 | 7.919 | −23.855 | 1.00 | 43.87 | B | C |
| ATOM | 4151 | CG | ASN | B | 244 | −9.999 | 8.560 | −25.011 | 1.00 | 47.13 | B | C |
| ATOM | 4152 | OD1 | ASN | B | 244 | −8.756 | 8.572 | −25.061 | 1.00 | 44.34 | B | O |
| ATOM | 4153 | ND2 | ASN | B | 244 | −10.748 | 9.112 | −25.957 | 1.00 | 51.75 | B | N |
| ATOM | 4154 | C | ASN | B | 244 | −10.921 | 6.076 | −22.180 | 1.00 | 38.39 | B | C |
| ATOM | 4155 | O | ASN | B | 244 | −11.998 | 5.629 | −22.522 | 1.00 | 40.30 | B | O |
| ATOM | 4156 | N | GLY | B | 245 | −10.462 | 5.995 | −20.936 | 1.00 | 34.92 | B | N |
| ATOM | 4157 | CA | GLY | B | 245 | −11.194 | 5.303 | −19.883 | 1.00 | 33.21 | B | C |
| ATOM | 4158 | C | GLY | B | 245 | −12.547 | 5.921 | −19.615 | 1.00 | 31.52 | B | C |
| ATOM | 4159 | O | GLY | B | 245 | −12.741 | 7.104 | −19.850 | 1.00 | 32.30 | B | O |
| ATOM | 4160 | N | ILE | B | 246 | −13.476 | 5.110 | −19.122 | 1.00 | 30.79 | B | N |
| ATOM | 4161 | CA | ILE | B | 246 | −14.800 | 5.578 | −18.734 | 1.00 | 31.70 | B | C |
| ATOM | 4162 | CB | ILE | B | 246 | −15.907 | 4.792 | −19.467 | 1.00 | 34.63 | B | C |
| ATOM | 4163 | CG1 | ILE | B | 246 | −15.952 | 3.312 | −19.041 | 1.00 | 35.25 | B | C |
| ATOM | 4164 | CD1 | ILE | B | 246 | −17.373 | 2.797 | −18.876 | 1.00 | 34.91 | B | C |
| ATOM | 4165 | CG2 | ILE | B | 246 | −15.708 | 4.884 | −20.976 | 1.00 | 35.49 | B | C |
| ATOM | 4166 | C | ILE | B | 246 | −15.056 | 5.495 | −17.231 | 1.00 | 31.36 | B | C |
| ATOM | 4167 | O | ILE | B | 246 | −16.153 | 5.815 | −16.784 | 1.00 | 29.30 | B | O |
| ATOM | 4168 | N | GLY | B | 247 | −14.038 | 5.076 | −16.469 | 1.00 | 33.15 | B | N |
| ATOM | 4169 | CA | GLY | B | 247 | −14.103 | 4.926 | −15.007 | 1.00 | 33.39 | B | C |
| ATOM | 4170 | C | GLY | B | 247 | −14.360 | 3.484 | −14.662 | 1.00 | 34.14 | B | C |
| ATOM | 4171 | O | GLY | B | 247 | −14.673 | 2.715 | −15.550 | 1.00 | 38.81 | B | O |
| ATOM | 4172 | N | ARG | B | 248 | −14.221 | 3.094 | −13.398 | 1.00 | 35.49 | B | N |
| ATOM | 4173 | CA | ARG | B | 248 | −14.521 | 1.701 | −13.019 | 1.00 | 34.52 | B | C |
| ATOM | 4174 | CB | ARG | B | 248 | −13.898 | 1.291 | −11.678 | 1.00 | 34.54 | B | C |
| ATOM | 4175 | CG | ARG | B | 248 | −12.394 | 1.119 | −11.670 | 1.00 | 33.03 | B | C |
| ATOM | 4176 | CD | ARG | B | 248 | −11.942 | 0.463 | −10.370 | 1.00 | 31.42 | B | C |
| ATOM | 4177 | NE | ARG | B | 248 | −10.484 | 0.407 | −10.297 | 1.00 | 30.41 | B | N |
| ATOM | 4178 | CZ | ARG | B | 248 | −9.772 | −0.522 | −9.665 | 1.00 | 30.13 | B | C |
| ATOM | 4179 | NH1 | ARG | B | 248 | −10.351 | −1.520 | −9.018 | 1.00 | 32.14 | B | N |
| ATOM | 4180 | NH2 | ARG | B | 248 | −8.455 | −0.457 | −9.676 | 1.00 | 29.38 | B | N |
| ATOM | 4181 | C | ARG | B | 248 | −16.014 | 1.528 | −12.922 | 1.00 | 34.18 | B | C |
| ATOM | 4182 | O | ARG | B | 248 | −16.606 | 0.839 | −13.724 | 1.00 | 35.53 | B | O |
| ATOM | 4183 | N | ASP | B | 249 | −16.620 | 2.168 | −11.935 | 1.00 | 35.34 | B | N |
| ATOM | 4184 | CA | ASP | B | 249 | −18.020 | 1.933 | −11.609 | 1.00 | 37.30 | B | C |
| ATOM | 4185 | CB | ASP | B | 249 | −18.584 | 3.108 | −10.808 | 1.00 | 39.37 | B | C |
| ATOM | 4186 | CG | ASP | B | 249 | −18.039 | 3.154 | −9.404 | 1.00 | 40.69 | B | C |
| ATOM | 4187 | OD1 | ASP | B | 249 | −18.225 | 2.155 | −8.689 | 1.00 | 41.67 | B | O |
| ATOM | 4188 | OD2 | ASP | B | 249 | −17.424 | 4.177 | −9.012 | 1.00 | 43.16 | B | O |
| ATOM | 4189 | C | ASP | B | 249 | −18.900 | 1.649 | −12.835 | 1.00 | 36.02 | B | C |
| ATOM | 4190 | O | ASP | B | 249 | −19.690 | 0.703 | −12.830 | 1.00 | 35.49 | B | O |
| ATOM | 4191 | N | ALA | B | 250 | −18.764 | 2.454 | −13.876 | 1.00 | 33.52 | B | N |
| ATOM | 4192 | CA | ALA | B | 250 | −19.556 | 2.228 | −15.067 | 1.00 | 34.12 | B | C |
| ATOM | 4193 | CB | ALA | B | 250 | −19.554 | 3.465 | −15.932 | 1.00 | 34.74 | B | C |
| ATOM | 4194 | C | ALA | B | 250 | −19.078 | 1.005 | −15.872 | 1.00 | 34.00 | B | C |
| ATOM | 4195 | O | ALA | B | 250 | −19.898 | 0.291 | −16.453 | 1.00 | 33.32 | B | O |
| ATOM | 4196 | N | ALA | B | 251 | −17.764 | 0.775 | −15.914 | 1.00 | 33.23 | B | N |
| ATOM | 4197 | CA | ALA | B | 251 | −17.192 | −0.376 | −16.634 | 1.00 | 32.65 | B | C |
| ATOM | 4198 | CB | ALA | B | 251 | −15.680 | −0.332 | −16.595 | 1.00 | 31.92 | B | C |
| ATOM | 4199 | C | ALA | B | 251 | −17.699 | −1.735 | −16.145 | 1.00 | 33.52 | B | C |
| ATOM | 4200 | O | ALA | B | 251 | −18.037 | −2.579 | −16.969 | 1.00 | 33.05 | B | O |
| ATOM | 4201 | N | VAL | B | 252 | −17.778 | −1.946 | −14.827 | 1.00 | 34.61 | B | N |
| ATOM | 4202 | CA | VAL | B | 252 | −18.321 | −3.223 | −14.299 | 1.00 | 37.23 | B | C |
| ATOM | 4203 | CB | VAL | B | 252 | −18.148 | −3.454 | −12.757 | 1.00 | 38.13 | B | C |
| ATOM | 4204 | CG1 | VAL | B | 252 | −16.734 | −3.132 | −12.298 | 1.00 | 39.21 | B | C |
| ATOM | 4205 | CG2 | VAL | B | 252 | −19.148 | −2.657 | −11.925 | 1.00 | 39.85 | B | C |
| ATOM | 4206 | C | VAL | B | 252 | −19.791 | −3.316 | −14.626 | 1.00 | 38.70 | B | C |
| ATOM | 4207 | O | VAL | B | 252 | −20.309 | −4.393 | −14.881 | 1.00 | 42.70 | B | O |
| ATOM | 4208 | N | GLN | B | 253 | −20.458 | −2.170 | −14.596 | 1.00 | 39.66 | B | N |
| ATOM | 4209 | CA | GLN | B | 253 | −21.882 | −2.101 | −14.842 | 1.00 | 38.93 | B | C |
| ATOM | 4210 | CB | GLN | B | 253 | −22.363 | −0.650 | −14.690 | 1.00 | 39.08 | B | C |
| ATOM | 4211 | CG | GLN | B | 253 | −23.849 | −0.455 | −14.817 | 1.00 | 38.82 | B | C |
| ATOM | 4212 | CD | GLN | B | 253 | −24.597 | −1.398 | −13.925 | 1.00 | 40.50 | B | C |
| ATOM | 4213 | OE1 | GLN | B | 253 | −25.255 | −2.330 | −14.399 | 1.00 | 41.81 | B | O |
| ATOM | 4214 | NE2 | GLN | B | 253 | −24.472 | −1.195 | −12.621 | 1.00 | 39.98 | B | N |
| ATOM | 4215 | C | GLN | B | 253 | −22.146 | −2.631 | −16.242 | 1.00 | 38.11 | B | C |
| ATOM | 4216 | O | GLN | B | 253 | −23.110 | −3.345 | −16.462 | 1.00 | 39.28 | B | O |
| ATOM | 4217 | N | ILE | B | 254 | −21.255 | −2.307 | −17.171 | 1.00 | 37.48 | B | N |
| ATOM | 4218 | CA | ILE | B | 254 | −21.358 | −2.789 | −18.537 | 1.00 | 37.65 | B | C |
| ATOM | 4219 | CB | ILE | B | 254 | −20.279 | −2.163 | −19.426 | 1.00 | 37.54 | B | C |
| ATOM | 4220 | CG1 | ILE | B | 254 | −20.665 | −0.718 | −19.712 | 1.00 | 38.79 | B | C |
| ATOM | 4221 | CD1 | ILE | B | 254 | −19.526 | 0.104 | −20.271 | 1.00 | 40.86 | B | C |
| ATOM | 4222 | CG2 | ILE | B | 254 | −20.068 | −2.972 | −20.714 | 1.00 | 36.04 | B | C |
| ATOM | 4223 | C | ILE | B | 254 | −21.210 | −4.292 | −18.625 | 1.00 | 38.14 | B | C |
| ATOM | 4224 | O | ILE | B | 254 | −21.986 | −4.948 | −19.323 | 1.00 | 38.29 | B | O |
| ATOM | 4225 | N | PHE | B | 255 | −20.194 | −4.825 | −17.950 | 1.00 | 37.29 | B | N |
| ATOM | 4226 | CA | PHE | B | 255 | −19.928 | −6.270 | −17.980 | 1.00 | 35.20 | B | C |
| ATOM | 4227 | CB | PHE | B | 255 | −18.526 | −6.589 | −17.464 | 1.00 | 34.13 | B | C |
| ATOM | 4228 | CG | PHE | B | 255 | −17.464 | −6.328 | −18.483 | 1.00 | 33.10 | B | C |
| ATOM | 4229 | CD1 | PHE | B | 255 | −16.910 | −5.080 | −18.618 | 1.00 | 33.11 | B | C |
| ATOM | 4230 | CE1 | PHE | B | 255 | −15.939 | −4.847 | −19.585 | 1.00 | 34.35 | B | C |
| ATOM | 4231 | CZ | PHE | B | 255 | −15.532 | −5.870 | −20.437 | 1.00 | 32.78 | B | C |
| ATOM | 4232 | CE2 | PHE | B | 255 | −16.107 | −7.110 | −20.329 | 1.00 | 31.36 | B | C |
| ATOM | 4233 | CD2 | PHE | B | 255 | −17.064 | −7.332 | −19.355 | 1.00 | 32.90 | B | C |
| ATOM | 4234 | C | PHE | B | 255 | −20.993 | −7.089 | −17.266 | 1.00 | 33.97 | B | C |
| ATOM | 4235 | O | PHE | B | 255 | −21.437 | −8.090 | −17.805 | 1.00 | 33.61 | B | O |
| ATOM | 4236 | N | TYR | B | 256 | −21.439 | −6.656 | −16.091 | 1.00 | 33.92 | B | N |
| ATOM | 4237 | CA | TYR | B | 256 | −22.584 | −7.298 | −15.444 | 1.00 | 33.80 | B | C |
| ATOM | 4238 | CB | TYR | B | 256 | −22.962 | −6.573 | −14.186 | 1.00 | 32.62 | B | C |
| ATOM | 4239 | CG | TYR | B | 256 | −24.124 | −7.165 | −13.423 | 1.00 | 32.49 | B | C |
| ATOM | 4240 | CD1 | TYR | B | 256 | −25.450 | −6.822 | −13.740 | 1.00 | 31.86 | B | C |
| ATOM | 4241 | CE1 | TYR | B | 256 | −26.517 | −7.342 | −13.024 | 1.00 | 31.32 | B | C |
| ATOM | 4242 | CZ | TYR | B | 256 | −26.269 | −8.192 | −11.946 | 1.00 | 32.70 | B | C |
| ATOM | 4243 | OH | TYR | B | 256 | −27.318 | −8.694 | −11.211 | 1.00 | 32.98 | B | O |
| ATOM | 4244 | CE2 | TYR | B | 256 | −24.963 | −8.522 | −11.588 | 1.00 | 32.52 | B | C |
| ATOM | 4245 | CD2 | TYR | B | 256 | −23.903 | −8.012 | −12.328 | 1.00 | 32.27 | B | C |
| ATOM | 4246 | C | TYR | B | 256 | −23.783 | −7.300 | −16.360 | 1.00 | 36.90 | B | C |
| ATOM | 4247 | O | TYR | B | 256 | −24.413 | −8.325 | −16.534 | 1.00 | 41.30 | B | O |
| ATOM | 4248 | N | ASP | B | 257 | −24.117 | −6.160 | −16.951 | 1.00 | 39.22 | B | N |
| ATOM | 4249 | CA | ASP | B | 257 | −25.285 | −6.114 | −17.840 | 1.00 | 40.23 | B | C |
| ATOM | 4250 | CB | ASP | B | 257 | −25.574 | −4.679 | −18.327 | 1.00 | 42.15 | B | C |
| ATOM | 4251 | CG | ASP | B | 257 | −26.329 | −3.819 | −17.279 | 1.00 | 43.41 | B | C |
| ATOM | 4252 | OD1 | ASP | B | 257 | −26.693 | −4.314 | −16.188 | 1.00 | 41.38 | B | O |
| ATOM | 4253 | OD2 | ASP | B | 257 | −26.567 | −2.623 | −17.563 | 1.00 | 46.53 | B | O |
| ATOM | 4254 | C | ASP | B | 257 | −25.131 | −7.101 | −19.013 | 1.00 | 38.49 | B | C |
| ATOM | 4255 | O | ASP | B | 257 | −26.062 | −7.828 | −19.341 | 1.00 | 37.98 | B | O |
| ATOM | 4256 | N | ALA | B | 258 | −23.949 | −7.150 | −19.616 | 1.00 | 37.63 | B | N |
| ATOM | 4257 | CA | ALA | B | 258 | −23.694 | −8.082 | −20.718 | 1.00 | 37.54 | B | C |
| ATOM | 4258 | CB | ALA | B | 258 | −22.317 | −7.832 | −21.331 | 1.00 | 37.74 | B | C |
| ATOM | 4259 | C | ALA | B | 258 | −23.833 | −9.547 | −20.274 | 1.00 | 36.90 | B | C |
| ATOM | 4260 | O | ALA | B | 258 | −24.351 | −10.371 | −21.013 | 1.00 | 37.34 | B | O |
| ATOM | 4261 | N | LEU | B | 259 | −23.373 | −9.849 | −19.067 | 1.00 | 35.49 | B | N |
| ATOM | 4262 | CA | LEU | B | 259 | −23.468 | −11.194 | −18.485 | 1.00 | 36.20 | B | C |
| ATOM | 4263 | CB | LEU | B | 259 | −22.692 | −11.243 | −17.149 | 1.00 | 35.03 | B | C |
| ATOM | 4264 | CG | LEU | B | 259 | −22.789 | −12.477 | −16.249 | 1.00 | 33.50 | B | C |
| ATOM | 4265 | CD1 | LEU | B | 259 | −21.969 | −13.599 | −16.855 | 1.00 | 34.00 | B | C |
| ATOM | 4266 | CD2 | LEU | B | 259 | −22.320 | −12.197 | −14.830 | 1.00 | 32.11 | B | C |
| ATOM | 4267 | C | LEU | B | 259 | −24.910 | −11.618 | −18.227 | 1.00 | 37.29 | B | C |
| ATOM | 4268 | O | LEU | B | 259 | −25.238 | −12.795 | −18.305 | 1.00 | 37.07 | B | O |
| ATOM | 4269 | N | ILE | B | 260 | −25.760 | −10.662 | −17.889 | 1.00 | 41.20 | B | N |
| ATOM | 4270 | CA | ILE | B | 260 | −27.138 | −10.968 | −17.499 | 1.00 | 45.53 | B | C |
| ATOM | 4271 | CB | ILE | B | 260 | −27.679 | −9.922 | −16.468 | 1.00 | 47.96 | B | C |
| ATOM | 4272 | CG1 | ILE | B | 260 | −27.242 | −10.273 | −15.049 | 1.00 | 49.37 | B | C |
| ATOM | 4273 | CD1 | ILE | B | 260 | −25.754 | −10.302 | −14.809 | 1.00 | 50.87 | B | C |
| ATOM | 4274 | CG2 | ILE | B | 260 | −29.203 | −9.896 | −16.425 | 1.00 | 50.61 | B | C |
| ATOM | 4275 | C | ILE | B | 260 | −28.037 | −11.059 | −18.746 | 1.00 | 41.96 | B | C |
| ATOM | 4276 | O | ILE | B | 260 | −29.047 | −11.766 | −18.741 | 1.00 | 37.45 | B | O |
| ATOM | 4277 | N | ASN | B | 261 | −27.647 | −10.353 | −19.806 | 1.00 | 41.28 | B | N |
| ATOM | 4278 | CA | ASN | B | 261 | −28.562 | −10.065 | −20.900 | 1.00 | 42.92 | B | C |
| ATOM | 4279 | CB | ASN | B | 261 | −28.929 | −8.555 | −20.935 | 1.00 | 41.62 | B | C |
| ATOM | 4280 | CG | ASN | B | 261 | −29.766 | −8.108 | −19.723 | 1.00 | 42.05 | B | C |
| ATOM | 4281 | OD1 | ASN | B | 261 | −29.472 | −7.101 | −19.087 | 1.00 | 39.28 | B | O |
| ATOM | 4282 | ND2 | ASN | B | 261 | −30.817 | −8.854 | −19.410 | 1.00 | 44.78 | B | N |
| ATOM | 4283 | C | ASN | B | 261 | −28.086 | −10.509 | −22.266 | 1.00 | 42.09 | B | C |
| ATOM | 4284 | O | ASN | B | 261 | −28.894 | −10.541 | −23.181 | 1.00 | 46.04 | B | O |
| ATOM | 4285 | N | TYR | B | 262 | −26.811 | −10.840 | −22.437 | 1.00 | 40.74 | B | N |
| ATOM | 4286 | CA | TYR | B | 262 | −26.328 | −11.224 | −23.775 | 1.00 | 43.37 | B | C |
| ATOM | 4287 | CB | TYR | B | 262 | −25.645 | −10.035 | −24.471 | 1.00 | 45.46 | B | C |
| ATOM | 4288 | CG | TYR | B | 262 | −26.615 | −8.895 | −24.720 | 1.00 | 50.69 | B | C |
| ATOM | 4289 | CD1 | TYR | B | 262 | −27.687 | −9.048 | −25.610 | 1.00 | 53.20 | B | C |
| ATOM | 4290 | CE1 | TYR | B | 262 | −28.603 | −8.023 | −25.824 | 1.00 | 51.02 | B | C |
| ATOM | 4291 | CZ | TYR | B | 262 | −28.459 | −6.829 | −25.145 | 1.00 | 50.83 | B | C |
| ATOM | 4292 | OH | TYR | B | 262 | −29.355 | −5.817 | −25.374 | 1.00 | 49.75 | B | O |
| ATOM | 4293 | CE2 | TYR | B | 262 | −27.406 | −6.644 | −24.259 | 1.00 | 51.59 | B | C |
| ATOM | 4294 | CD2 | TYR | B | 262 | −26.497 | −7.676 | −24.040 | 1.00 | 52.01 | B | C |
| ATOM | 4295 | C | TYR | B | 262 | −25.448 | −12.478 | −23.816 | 1.00 | 42.34 | B | C |
| ATOM | 4296 | O | TYR | B | 262 | −25.698 | −13.358 | −24.629 | 1.00 | 40.29 | B | O |
| ATOM | 4297 | N | LEU | B | 263 | −24.442 | −12.568 | −22.943 | 1.00 | 42.79 | B | N |
| ATOM | 4298 | CA | LEU | B | 263 | −23.480 | −13.686 | −22.966 | 1.00 | 40.87 | B | C |
| ATOM | 4299 | CB | LEU | B | 263 | −22.404 | −13.522 | −21.890 | 1.00 | 39.39 | B | C |
| ATOM | 4300 | CG | LEU | B | 263 | −21.392 | −12.379 | −22.072 | 1.00 | 39.16 | B | C |
| ATOM | 4301 | CD1 | LEU | B | 263 | −20.636 | −12.135 | −20.758 | 1.00 | 38.76 | B | C |
| ATOM | 4302 | CD2 | LEU | B | 263 | −20.439 | −12.620 | −23.251 | 1.00 | 36.77 | B | C |
| ATOM | 4303 | C | LEU | B | 263 | −24.182 | −15.025 | −22.784 | 1.00 | 41.44 | B | C |
| ATOM | 4304 | O | LEU | B | 263 | −24.950 | −15.224 | −21.830 | 1.00 | 43.17 | B | O |
| ATOM | 4305 | N | THR | B | 264 | −23.920 | −15.937 | −23.710 | 1.00 | 39.41 | B | N |
| ATOM | 4306 | CA | THR | B | 264 | −24.539 | −17.252 | −23.678 | 1.00 | 39.11 | B | C |
| ATOM | 4307 | CB | THR | B | 264 | −25.110 | −17.637 | −25.071 | 1.00 | 37.81 | B | C |
| ATOM | 4308 | OG1 | THR | B | 264 | −24.209 | −17.240 | −26.114 | 1.00 | 34.47 | B | O |
| ATOM | 4309 | CG2 | THR | B | 264 | −26.464 | −16.980 | −25.291 | 1.00 | 37.47 | B | C |
| ATOM | 4310 | C | THR | B | 264 | −23.516 | −18.289 | −23.180 | 1.00 | 39.71 | B | C |
| ATOM | 4311 | O | THR | B | 264 | −22.332 | −17.968 | −22.988 | 1.00 | 39.63 | B | O |
| ATOM | 4312 | N | PRO | B | 265 | −23.967 | −19.534 | −22.956 | 1.00 | 37.47 | B | N |
| ATOM | 4313 | CA | PRO | B | 265 | −23.020 | −20.582 | −22.606 | 1.00 | 36.51 | B | C |
| ATOM | 4314 | CB | PRO | B | 265 | −23.914 | −21.815 | −22.485 | 1.00 | 36.96 | B | C |
| ATOM | 4315 | CG | PRO | B | 265 | −25.212 | −21.258 | −21.998 | 1.00 | 36.90 | B | C |
| ATOM | 4316 | CD | PRO | B | 265 | −25.362 | −19.978 | −22.764 | 1.00 | 37.02 | B | C |
| ATOM | 4317 | C | PRO | B | 265 | −21.889 | −20.826 | −23.602 | 1.00 | 34.86 | B | C |
| ATOM | 4318 | O | PRO | B | 265 | −20.886 | −21.386 | −23.218 | 1.00 | 34.81 | B | O |
| ATOM | 4319 | N | THR | B | 266 | −22.029 | −20.371 | −24.836 | 1.00 | 36.32 | B | N |
| ATOM | 4320 | CA | THR | B | 266 | −21.064 | −20.660 | −25.909 | 1.00 | 38.58 | B | C |
| ATOM | 4321 | CB | THR | B | 266 | −21.832 | −21.113 | −27.172 | 1.00 | 39.51 | B | C |
| ATOM | 4322 | OG1 | THR | B | 266 | −22.792 | −22.103 | −26.783 | 1.00 | 40.49 | B | O |
| ATOM | 4323 | CG2 | THR | B | 266 | −20.891 | −21.667 | −28.272 | 1.00 | 39.24 | B | C |
| ATOM | 4324 | C | THR | B | 266 | −20.208 | −19.477 | −26.339 | 1.00 | 37.79 | B | C |
| ATOM | 4325 | O | THR | B | 266 | −19.415 | −19.606 | −27.267 | 1.00 | 40.23 | B | O |
| ATOM | 4326 | N | SER | B | 267 | −20.369 | −18.324 | −25.697 | 1.00 | 36.30 | B | N |
| ATOM | 4327 | CA | SER | B | 267 | −19.731 | −17.102 | −26.183 | 1.00 | 34.28 | B | C |
| ATOM | 4328 | CB | SER | B | 267 | −20.113 | −15.912 | −25.319 | 1.00 | 35.49 | B | C |
| ATOM | 4329 | OG | SER | B | 267 | −21.451 | −16.033 | −24.828 | 1.00 | 37.99 | B | O |
| ATOM | 4330 | C | SER | B | 267 | −18.230 | −17.251 | −26.190 | 1.00 | 32.77 | B | C |
| ATOM | 4331 | O | SER | B | 267 | −17.641 | −17.643 | −25.208 | 1.00 | 33.92 | B | O |
| ATOM | 4332 | N | ASN | B | 268 | −17.620 | −16.986 | −27.326 | 1.00 | 32.61 | B | N |
| ATOM | 4333 | CA | ASN | B | 268 | −16.182 | −16.863 | −27.415 | 1.00 | 33.06 | B | C |
| ATOM | 4334 | CB | ASN | B | 268 | −15.729 | −17.371 | −28.773 | 1.00 | 33.54 | B | C |
| ATOM | 4335 | CG | ASN | B | 268 | −16.267 | −16.536 | −29.895 | 1.00 | 33.79 | B | C |
| ATOM | 4336 | OD1 | ASN | B | 268 | −17.326 | −15.949 | −29.777 | 1.00 | 35.97 | B | O |
| ATOM | 4337 | ND2 | ASN | B | 268 | −15.540 | −16.461 | −30.975 | 1.00 | 34.73 | B | N |
| ATOM | 4338 | C | ASN | B | 268 | −15.787 | −15.390 | −27.231 | 1.00 | 34.80 | B | C |
| ATOM | 4339 | O | ASN | B | 268 | −16.626 | −14.538 | −26.913 | 1.00 | 34.48 | B | O |
| ATOM | 4340 | N | PHE | B | 269 | −14.514 | −15.079 | −27.443 | 1.00 | 36.91 | B | N |
| ATOM | 4341 | CA | PHE | B | 269 | −14.026 | −13.738 | −27.157 | 1.00 | 38.91 | B | C |
| ATOM | 4342 | CB | PHE | B | 269 | −12.507 | −13.628 | −27.385 | 1.00 | 40.79 | B | C |
| ATOM | 4343 | CG | PHE | B | 269 | −11.681 | −13.874 | −26.141 | 1.00 | 41.78 | B | C |
| ATOM | 4344 | CD1 | PHE | B | 269 | −11.418 | −12.849 | −25.251 | 1.00 | 41.82 | B | C |
| ATOM | 4345 | CE1 | PHE | B | 269 | −10.654 | −13.078 | −24.112 | 1.00 | 41.04 | B | C |
| ATOM | 4346 | CZ | PHE | B | 269 | −10.135 | −14.332 | −23.857 | 1.00 | 38.55 | B | C |
| ATOM | 4347 | CE2 | PHE | B | 269 | −10.382 | −15.356 | −24.732 | 1.00 | 38.41 | B | C |
| ATOM | 4348 | CD2 | PHE | B | 269 | −11.155 | −15.130 | −25.867 | 1.00 | 41.11 | B | C |
| ATOM | 4349 | C | PHE | B | 269 | −14.805 | −12.684 | −27.957 | 1.00 | 38.14 | B | C |
| ATOM | 4350 | O | PHE | B | 269 | −15.419 | −11.804 | −27.371 | 1.00 | 37.56 | B | O |
| ATOM | 4351 | N | SER | B | 270 | −14.842 | −12.803 | −29.281 | 1.00 | 36.64 | B | N |
| ATOM | 4352 | CA | SER | B | 270 | −15.531 | −11.802 | −30.090 | 1.00 | 34.60 | B | C |
| ATOM | 4353 | CB | SER | B | 270 | −14.897 | −11.695 | −31.499 | 1.00 | 34.71 | B | C |
| ATOM | 4354 | OG | SER | B | 270 | −15.617 | −12.383 | −32.491 | 1.00 | 34.03 | B | O |
| ATOM | 4355 | C | SER | B | 270 | −17.045 | −12.041 | −30.114 | 1.00 | 32.63 | B | C |
| ATOM | 4356 | O | SER | B | 270 | −17.674 | −11.999 | −31.146 | 1.00 | 36.29 | B | O |
| ATOM | 4357 | N | ALA | B | 271 | −17.604 | −12.349 | −28.960 | 1.00 | 30.68 | B | N |
| ATOM | 4358 | CA | ALA | B | 271 | −19.038 | −12.294 | −28.730 | 1.00 | 30.92 | B | C |
| ATOM | 4359 | CB | ALA | B | 271 | −19.613 | −13.701 | −28.600 | 1.00 | 30.65 | B | C |
| ATOM | 4360 | C | ALA | B | 271 | −19.247 | −11.507 | −27.444 | 1.00 | 30.93 | B | C |
| ATOM | 4361 | O | ALA | B | 271 | −20.307 | −10.917 | −27.213 | 1.00 | 29.36 | B | O |
| ATOM | 4362 | N | MET | B | 272 | −18.229 | −11.586 | −26.584 | 1.00 | 32.20 | B | N |
| ATOM | 4363 | CA | MET | B | 272 | −18.031 | −10.679 | −25.460 | 1.00 | 32.76 | B | C |
| ATOM | 4364 | CB | MET | B | 272 | −16.756 | −11.071 | −24.673 | 1.00 | 32.48 | B | C |
| ATOM | 4365 | CG | MET | B | 272 | −16.360 | −10.134 | −23.533 | 1.00 | 31.70 | B | C |
| ATOM | 4366 | SD | MET | B | 272 | −17.286 | −10.362 | −22.001 | 1.00 | 31.29 | B | S |
| ATOM | 4367 | CE | MET | B | 272 | −18.784 | −9.421 | −22.293 | 1.00 | 30.09 | B | C |
| ATOM | 4368 | C | MET | B | 272 | −17.923 | −9.252 | −25.975 | 1.00 | 32.88 | B | C |
| ATOM | 4369 | O | MET | B | 272 | −18.662 | −8.395 | −25.515 | 1.00 | 32.30 | B | O |
| ATOM | 4370 | N | ARG | B | 273 | −17.011 | −9.021 | −26.933 | 1.00 | 34.73 | B | N |
| ATOM | 4371 | CA | ARG | B | 273 | −16.906 | −7.739 | −27.658 | 1.00 | 35.03 | B | C |
| ATOM | 4372 | CB | ARG | B | 273 | −16.032 | −7.842 | −28.925 | 1.00 | 34.92 | B | C |
| ATOM | 4373 | CG | ARG | B | 273 | −15.783 | −6.483 | −29.582 | 1.00 | 36.57 | B | C |
| ATOM | 4374 | CD | ARG | B | 273 | −15.352 | −6.494 | −31.046 | 1.00 | 38.56 | B | C |
| ATOM | 4375 | NE | ARG | B | 273 | −15.489 | −5.154 | −31.660 | 1.00 | 40.10 | B | N |
| ATOM | 4376 | CZ | ARG | B | 273 | −14.486 | −4.289 | −31.894 | 1.00 | 40.63 | B | C |
| ATOM | 4377 | NH1 | ARG | B | 273 | −13.218 | −4.572 | −31.590 | 1.00 | 39.02 | B | N |
| ATOM | 4378 | NH2 | ARG | B | 273 | −14.752 | −3.107 | −32.442 | 1.00 | 41.17 | B | N |
| ATOM | 4379 | C | ARG | B | 273 | −18.295 | −7.267 | −28.039 | 1.00 | 34.93 | B | C |
| ATOM | 4380 | O | ARG | B | 273 | −18.770 | −6.252 | −27.549 | 1.00 | 34.29 | B | O |
| ATOM | 4381 | N | ALA | B | 274 | −18.955 | −8.045 | −28.884 | 1.00 | 37.65 | B | N |
| ATOM | 4382 | CA | ALA | B | 274 | −20.304 | −7.717 | −29.344 | 1.00 | 37.88 | B | C |
| ATOM | 4383 | CB | ALA | B | 274 | −20.886 | −8.866 | −30.184 | 1.00 | 38.03 | B | C |
| ATOM | 4384 | C | ALA | B | 274 | −21.217 | −7.383 | −28.170 | 1.00 | 33.95 | B | C |
| ATOM | 4385 | O | ALA | B | 274 | −21.822 | −6.319 | −28.149 | 1.00 | 32.98 | B | O |
| ATOM | 4386 | N | ALA | B | 275 | −21.296 | −8.285 | −27.200 | 1.00 | 32.07 | B | N |
| ATOM | 4387 | CA | ALA | B | 275 | −22.201 | −8.106 | −26.070 | 1.00 | 33.08 | B | C |
| ATOM | 4388 | CB | ALA | B | 275 | −22.345 | −9.407 | −25.299 | 1.00 | 32.37 | B | C |
| ATOM | 4389 | C | ALA | B | 275 | −21.789 | −6.962 | −25.127 | 1.00 | 34.71 | B | C |
| ATOM | 4390 | O | ALA | B | 275 | −22.640 | −6.437 | −24.384 | 1.00 | 33.51 | B | O |
| ATOM | 4391 | N | ALA | B | 276 | −20.505 | −6.581 | −25.166 | 1.00 | 34.12 | B | N |
| ATOM | 4392 | CA | ALA | B | 276 | −19.991 | −5.442 | −24.381 | 1.00 | 34.35 | B | C |
| ATOM | 4393 | CB | ALA | B | 276 | −18.467 | −5.428 | −24.360 | 1.00 | 34.79 | B | C |
| ATOM | 4394 | C | ALA | B | 276 | −20.486 | −4.140 | −24.970 | 1.00 | 33.42 | B | C |
| ATOM | 4395 | O | ALA | B | 276 | −20.986 | −3.249 | −24.258 | 1.00 | 30.24 | B | O |
| ATOM | 4396 | N | ILE | B | 277 | −20.313 | −4.046 | −26.284 | 1.00 | 33.82 | B | N |
| ATOM | 4397 | CA | ILE | B | 277 | −20.893 | −2.967 | −27.069 | 1.00 | 34.25 | B | C |
| ATOM | 4398 | CB | ILE | B | 277 | −20.669 | −3.167 | −28.584 | 1.00 | 32.40 | B | C |
| ATOM | 4399 | CG1 | ILE | B | 277 | −19.176 | −2.974 | −28.939 | 1.00 | 30.53 | B | C |
| ATOM | 4400 | CD1 | ILE | B | 277 | −18.811 | −3.402 | −30.343 | 1.00 | 30.29 | B | C |
| ATOM | 4401 | CG2 | ILE | B | 277 | −21.570 | −2.220 | −29.370 | 1.00 | 32.72 | B | C |
| ATOM | 4402 | C | ILE | B | 277 | −22.376 | −2.909 | −26.750 | 1.00 | 35.55 | B | C |
| ATOM | 4403 | O | ILE | B | 277 | −22.852 | −1.988 | −26.123 | 1.00 | 37.39 | B | O |
| ATOM | 4404 | N | GLN | B | 278 | −23.092 | −3.943 | −27.119 | 1.00 | 38.65 | B | N |
| ATOM | 4405 | CA | GLN | B | 278 | −24.511 | −3.926 | −26.950 | 1.00 | 39.92 | B | C |
| ATOM | 4406 | CB | GLN | B | 278 | −25.094 | −5.290 | −27.321 | 1.00 | 39.74 | B | C |
| ATOM | 4407 | CG | GLN | B | 278 | −26.606 | −5.300 | −27.308 | 1.00 | 39.67 | B | C |
| ATOM | 4408 | CD | GLN | B | 278 | −27.189 | −4.111 | −28.042 | 1.00 | 38.74 | B | C |
| ATOM | 4409 | OE1 | GLN | B | 278 | −26.710 | −3.731 | −29.109 | 1.00 | 35.78 | B | O |
| ATOM | 4410 | NE2 | GLN | B | 278 | −28.208 | −3.499 | −27.453 | 1.00 | 38.39 | B | N |
| ATOM | 4411 | C | GLN | B | 278 | −24.965 | −3.489 | −25.546 | 1.00 | 42.36 | B | C |
| ATOM | 4412 | O | GLN | B | 278 | −25.940 | −2.757 | −25.439 | 1.00 | 48.71 | B | O |
| ATOM | 4413 | N | ALA | B | 279 | −24.285 | −3.905 | −24.478 | 1.00 | 45.05 | B | N |
| ATOM | 4414 | CA | ALA | B | 279 | −24.730 | −3.520 | −23.108 | 1.00 | 46.98 | B | C |
| ATOM | 4415 | CB | ALA | B | 279 | −24.072 | −4.393 | −22.055 | 1.00 | 46.66 | B | C |
| ATOM | 4416 | C | ALA | B | 279 | −24.469 | −2.039 | −22.790 | 1.00 | 48.10 | B | C |
| ATOM | 4417 | O | ALA | B | 279 | −25.275 | −1.393 | −22.123 | 1.00 | 45.73 | B | O |
| ATOM | 4418 | N | ALA | B | 280 | −23.325 | −1.529 | −23.261 | 1.00 | 50.53 | B | N |
| ATOM | 4419 | CA | ALA | B | 280 | −22.959 | −0.109 | −23.139 | 1.00 | 49.16 | B | C |
| ATOM | 4420 | CB | ALA | B | 280 | −21.503 | 0.098 | −23.531 | 1.00 | 48.26 | B | C |
| ATOM | 4421 | C | ALA | B | 280 | −23.850 | 0.770 | −24.006 | 1.00 | 50.26 | B | C |
| ATOM | 4422 | O | ALA | B | 280 | −24.128 | 1.910 | −23.647 | 1.00 | 55.46 | B | O |
| ATOM | 4423 | N | THR | B | 281 | −24.272 | 0.236 | −25.150 | 1.00 | 46.81 | B | N |
| ATOM | 4424 | CA | THR | B | 281 | −25.211 | 0.899 | −26.024 | 1.00 | 45.54 | B | C |
| ATOM | 4425 | CB | THR | B | 281 | −25.317 | 0.164 | −27.381 | 1.00 | 47.69 | B | C |
| ATOM | 4426 | OG1 | THR | B | 281 | −24.123 | 0.397 | −28.143 | 1.00 | 48.16 | B | O |
| ATOM | 4427 | CG2 | THR | B | 281 | −26.488 | 0.648 | −28.210 | 1.00 | 48.37 | B | C |
| ATOM | 4428 | C | THR | B | 281 | −26.557 | 1.023 | −25.310 | 1.00 | 47.32 | B | C |
| ATOM | 4429 | O | THR | B | 281 | −27.094 | 2.131 | −25.213 | 1.00 | 49.73 | B | O |
| ATOM | 4430 | N | ASP | B | 282 | −27.081 | −0.081 | −24.771 | 1.00 | 47.65 | B | N |
| ATOM | 4431 | CA | ASP | B | 282 | −28.330 | −0.052 | −23.961 | 1.00 | 49.02 | B | C |
| ATOM | 4432 | CB | ASP | B | 282 | −28.609 | −1.428 | −23.343 | 1.00 | 49.93 | B | C |
| ATOM | 4433 | CG | ASP | B | 282 | −29.000 | −2.464 | −24.366 | 1.00 | 51.23 | B | C |
| ATOM | 4434 | OD1 | ASP | B | 282 | −29.308 | −2.090 | −25.518 | 1.00 | 47.87 | B | O |
| ATOM | 4435 | OD2 | ASP | B | 282 | −28.995 | −3.662 | −24.005 | 1.00 | 53.69 | B | O |
| ATOM | 4436 | C | ASP | B | 282 | −28.359 | 0.976 | −22.811 | 1.00 | 49.83 | B | C |
| ATOM | 4437 | O | ASP | B | 282 | −29.439 | 1.378 | −22.350 | 1.00 | 47.84 | B | O |
| ATOM | 4438 | N | LEU | B | 283 | −27.177 | 1.363 | −22.329 | 1.00 | 51.88 | B | N |
| ATOM | 4439 | CA | LEU | B | 283 | −27.052 | 2.309 | −21.218 | 1.00 | 52.09 | B | C |
| ATOM | 4440 | CB | LEU | B | 283 | −26.006 | 1.820 | −20.213 | 1.00 | 52.89 | B | C |
| ATOM | 4441 | CG | LEU | B | 283 | −26.431 | 0.781 | −19.184 | 1.00 | 52.14 | B | C |
| ATOM | 4442 | CD1 | LEU | B | 283 | −25.348 | 0.710 | −18.117 | 1.00 | 51.63 | B | C |
| ATOM | 4443 | CD2 | LEU | B | 283 | −27.796 | 1.094 | −18.578 | 1.00 | 50.82 | B | C |
| ATOM | 4444 | C | LEU | B | 283 | −26.675 | 3.721 | −21.656 | 1.00 | 49.74 | B | C |
| ATOM | 4445 | O | LEU | B | 283 | −27.352 | 4.665 | −21.303 | 1.00 | 50.65 | B | O |
| ATOM | 4446 | N | TYR | B | 284 | −25.586 | 3.863 | −22.401 | 1.00 | 48.81 | B | N |
| ATOM | 4447 | CA | TYR | B | 284 | −25.061 | 5.189 | −22.727 | 1.00 | 49.44 | B | C |
| ATOM | 4448 | CB | TYR | B | 284 | −23.556 | 5.262 | −22.403 | 1.00 | 50.48 | B | C |
| ATOM | 4449 | CG | TYR | B | 284 | −23.236 | 4.737 | −21.026 | 1.00 | 49.15 | B | C |
| ATOM | 4450 | CD1 | TYR | B | 284 | −23.872 | 5.254 | −19.903 | 1.00 | 49.52 | B | C |
| ATOM | 4451 | CE1 | TYR | B | 284 | −23.603 | 4.762 | −18.632 | 1.00 | 50.75 | B | C |
| ATOM | 4452 | CZ | TYR | B | 284 | −22.681 | 3.732 | −18.471 | 1.00 | 50.38 | B | C |
| ATOM | 4453 | OH | TYR | B | 284 | −22.404 | 3.229 | −17.205 | 1.00 | 47.77 | B | O |
| ATOM | 4454 | CE2 | TYR | B | 284 | −22.048 | 3.197 | −19.584 | 1.00 | 49.51 | B | C |
| ATOM | 4455 | CD2 | TYR | B | 284 | −22.333 | 3.695 | −20.848 | 1.00 | 49.51 | B | C |
| ATOM | 4456 | C | TYR | B | 284 | −25.315 | 5.610 | −24.171 | 1.00 | 49.25 | B | C |
| ATOM | 4457 | O | TYR | B | 284 | −24.821 | 6.647 | −24.590 | 1.00 | 48.69 | B | O |
| ATOM | 4458 | N | GLY | B | 285 | −26.074 | 4.821 | −24.930 | 1.00 | 50.48 | B | N |
| ATOM | 4459 | CA | GLY | B | 285 | −26.504 | 5.227 | −26.273 | 1.00 | 49.49 | B | C |
| ATOM | 4460 | C | GLY | B | 285 | −25.475 | 4.969 | −27.360 | 1.00 | 48.24 | B | C |
| ATOM | 4461 | O | GLY | B | 285 | −24.297 | 5.259 | −27.199 | 1.00 | 44.44 | B | O |
| ATOM | 4462 | N | ALA | B | 286 | −25.958 | 4.477 | −28.494 | 1.00 | 49.71 | B | N |
| ATOM | 4463 | CA | ALA | B | 286 | −25.129 | 3.842 | −29.533 | 1.00 | 53.31 | B | C |
| ATOM | 4464 | CB | ALA | B | 286 | −25.958 | 3.664 | −30.805 | 1.00 | 54.01 | B | C |
| ATOM | 4465 | C | ALA | B | 286 | −23.777 | 4.486 | −29.893 | 1.00 | 53.88 | B | C |
| ATOM | 4466 | O | ALA | B | 286 | −22.839 | 3.779 | −30.282 | 1.00 | 51.19 | B | O |
| ATOM | 4467 | N | ASN | B | 287 | −23.673 | 5.807 | −29.803 | 1.00 | 54.40 | B | N |
| ATOM | 4468 | CA | ASN | B | 287 | −22.489 | 6.464 | −30.315 | 1.00 | 57.64 | B | C |
| ATOM | 4469 | CB | ASN | B | 287 | −22.872 | 7.348 | −31.514 | 1.00 | 60.09 | B | C |
| ATOM | 4470 | CG | ASN | B | 287 | −22.568 | 6.674 | −32.851 | 1.00 | 60.57 | B | C |
| ATOM | 4471 | OD1 | ASN | B | 287 | −21.399 | 6.491 | −33.202 | 1.00 | 62.04 | B | O |
| ATOM | 4472 | ND2 | ASN | B | 287 | −23.610 | 6.296 | −33.598 | 1.00 | 57.39 | B | N |
| ATOM | 4473 | C | ASN | B | 287 | −21.741 | 7.215 | −29.227 | 1.00 | 58.94 | B | C |
| ATOM | 4474 | O | ASN | B | 287 | −21.301 | 8.344 | −29.423 | 1.00 | 58.57 | B | O |
| ATOM | 4475 | N | SER | B | 288 | −21.549 | 6.542 | −28.093 | 1.00 | 58.78 | B | N |
| ATOM | 4476 | CA | SER | B | 288 | −21.058 | 7.184 | −26.870 | 1.00 | 54.62 | B | C |
| ATOM | 4477 | CB | SER | B | 288 | −21.578 | 6.456 | −25.636 | 1.00 | 53.53 | B | C |
| ATOM | 4478 | OG | SER | B | 288 | −22.968 | 6.277 | −25.713 | 1.00 | 53.88 | B | O |
| ATOM | 4479 | C | SER | B | 288 | −19.554 | 7.219 | −26.756 | 1.00 | 52.14 | B | C |
| ATOM | 4480 | O | SER | B | 288 | −18.835 | 6.460 | −27.428 | 1.00 | 48.93 | B | O |
| ATOM | 4481 | N | SER | B | 289 | −19.101 | 8.113 | −25.877 | 1.00 | 49.50 | B | N |
| ATOM | 4482 | CA | SER | B | 289 | −17.748 | 8.063 | −25.338 | 1.00 | 48.18 | B | C |
| ATOM | 4483 | CB | SER | B | 289 | −17.658 | 8.897 | −24.062 | 1.00 | 46.70 | B | C |
| ATOM | 4484 | OG | SER | B | 289 | −17.504 | 10.261 | −24.344 | 1.00 | 47.82 | B | O |
| ATOM | 4485 | C | SER | B | 289 | −17.420 | 6.629 | −24.973 | 1.00 | 45.61 | B | C |
| ATOM | 4486 | O | SER | B | 289 | −16.363 | 6.096 | −25.322 | 1.00 | 42.65 | B | O |
| ATOM | 4487 | N | GLN | B | 290 | −18.371 | 6.033 | −24.264 | 1.00 | 44.11 | B | N |
| ATOM | 4488 | CA | GLN | B | 290 | −18.221 | 4.722 | −23.655 | 1.00 | 45.53 | B | C |
| ATOM | 4489 | CB | GLN | B | 290 | −19.422 | 4.420 | −22.743 | 1.00 | 46.86 | B | C |
| ATOM | 4490 | CG | GLN | B | 290 | −19.350 | 5.126 | −21.390 | 1.00 | 48.38 | B | C |
| ATOM | 4491 | CD | GLN | B | 290 | −19.890 | 6.548 | −21.405 | 1.00 | 46.61 | B | C |
| ATOM | 4492 | OE1 | GLN | B | 290 | −20.064 | 7.138 | −22.465 | 1.00 | 47.54 | B | O |
| ATOM | 4493 | NE2 | GLN | B | 290 | −20.156 | 7.098 | −20.221 | 1.00 | 44.66 | B | N |
| ATOM | 4494 | C | GLN | B | 290 | −18.081 | 3.619 | −24.674 | 1.00 | 43.32 | B | C |
| ATOM | 4495 | O | GLN | B | 290 | −17.141 | 2.838 | −24.621 | 1.00 | 41.88 | B | O |
| ATOM | 4496 | N | VAL | B | 291 | −19.029 | 3.562 | −25.594 | 1.00 | 41.50 | B | N |
| ATOM | 4497 | CA | VAL | B | 291 | −19.014 | 2.555 | −26.622 | 1.00 | 40.16 | B | C |
| ATOM | 4498 | CB | VAL | B | 291 | −20.266 | 2.679 | −27.505 | 1.00 | 39.26 | B | C |
| ATOM | 4499 | CG1 | VAL | B | 291 | −20.219 | 1.674 | −28.647 | 1.00 | 39.12 | B | C |
| ATOM | 4500 | CG2 | VAL | B | 291 | −21.524 | 2.494 | −26.651 | 1.00 | 38.82 | B | C |
| ATOM | 4501 | C | VAL | B | 291 | −17.714 | 2.639 | −27.432 | 1.00 | 41.56 | B | C |
| ATOM | 4502 | O | VAL | B | 291 | −17.078 | 1.624 | −27.664 | 1.00 | 41.96 | B | O |
| ATOM | 4503 | N | ASN | B | 292 | −17.303 | 3.847 | −27.821 | 1.00 | 47.66 | B | N |
| ATOM | 4504 | CA | ASN | B | 292 | −16.051 | 4.063 | −28.595 | 1.00 | 49.69 | B | C |
| ATOM | 4505 | CB | ASN | B | 292 | −15.801 | 5.576 | −28.850 | 1.00 | 56.17 | B | C |
| ATOM | 4506 | CG | ASN | B | 292 | −16.608 | 6.138 | −30.026 | 1.00 | 59.22 | B | C |
| ATOM | 4507 | OD1 | ASN | B | 292 | −16.441 | 5.711 | −31.179 | 1.00 | 58.08 | B | O |
| ATOM | 4508 | ND2 | ASN | B | 292 | −17.466 | 7.126 | −29.741 | 1.00 | 57.45 | B | N |
| ATOM | 4509 | C | ASN | B | 292 | −14.813 | 3.473 | −27.908 | 1.00 | 44.46 | B | C |
| ATOM | 4510 | O | ASN | B | 292 | −13.892 | 2.961 | −28.567 | 1.00 | 40.14 | B | O |
| ATOM | 4511 | N | ALA | B | 293 | −14.792 | 3.576 | −26.581 | 1.00 | 42.31 | B | N |
| ATOM | 4512 | CA | ALA | B | 293 | −13.730 | 2.972 | −25.763 | 1.00 | 41.98 | B | C |
| ATOM | 4513 | CB | ALA | B | 293 | −13.829 | 3.451 | −24.321 | 1.00 | 41.43 | B | C |
| ATOM | 4514 | C | ALA | B | 293 | −13.756 | 1.433 | −25.810 | 1.00 | 39.16 | B | C |
| ATOM | 4515 | O | ALA | B | 293 | −12.721 | 0.815 | −26.082 | 1.00 | 36.57 | B | O |
| ATOM | 4516 | N | VAL | B | 294 | −14.936 | 0.852 | −25.539 | 1.00 | 36.84 | B | N |
| ATOM | 4517 | CA | VAL | B | 294 | −15.177 | −0.595 | −25.610 | 1.00 | 35.25 | B | C |
| ATOM | 4518 | CB | VAL | B | 294 | −16.659 | −0.950 | −25.468 | 1.00 | 35.72 | B | C |
| ATOM | 4519 | CG1 | VAL | B | 294 | −16.878 | −2.433 | −25.729 | 1.00 | 37.88 | B | C |
| ATOM | 4520 | CG2 | VAL | B | 294 | −17.176 | −0.610 | −24.091 | 1.00 | 36.30 | B | C |
| ATOM | 4521 | C | VAL | B | 294 | −14.748 | −1.144 | −26.945 | 1.00 | 35.55 | B | C |
| ATOM | 4522 | O | VAL | B | 294 | −13.956 | −2.064 | −26.995 | 1.00 | 33.54 | B | O |
| ATOM | 4523 | N | LYS | B | 295 | −15.286 | −0.587 | −28.025 | 1.00 | 37.14 | B | N |
| ATOM | 4524 | CA | LYS | B | 295 | −14.869 | −0.983 | −29.360 | 1.00 | 41.15 | B | C |
| ATOM | 4525 | CB | LYS | B | 295 | −15.422 | −0.035 | −30.422 | 1.00 | 46.63 | B | C |
| ATOM | 4526 | CG | LYS | B | 295 | −16.913 | −0.162 | −30.660 | 1.00 | 52.98 | B | C |
| ATOM | 4527 | CD | LYS | B | 295 | −17.355 | 0.640 | −31.874 | 1.00 | 58.56 | B | C |
| ATOM | 4528 | CE | LYS | B | 295 | −18.834 | 0.407 | −32.159 | 1.00 | 64.17 | B | C |
| ATOM | 4529 | NZ | LYS | B | 295 | −19.271 | 1.113 | −33.395 | 1.00 | 67.47 | B | N |
| ATOM | 4530 | C | LYS | B | 295 | −13.372 | −0.957 | −29.481 | 1.00 | 39.83 | B | C |
| ATOM | 4531 | O | LYS | B | 295 | −12.768 | −1.874 | −30.035 | 1.00 | 41.50 | B | O |
| ATOM | 4532 | N | LYS | B | 296 | −12.786 | 0.118 | −28.972 | 1.00 | 40.95 | B | N |
| ATOM | 4533 | CA | LYS | B | 296 | −11.379 | 0.436 | −29.209 | 1.00 | 42.53 | B | C |
| ATOM | 4534 | CB | LYS | B | 296 | −11.161 | 1.962 | −29.047 | 1.00 | 43.71 | B | C |
| ATOM | 4535 | CG | LYS | B | 296 | −9.937 | 2.509 | −29.771 | 1.00 | 47.26 | B | C |
| ATOM | 4536 | CD | LYS | B | 296 | −10.251 | 3.670 | −30.724 | 1.00 | 50.59 | B | C |
| ATOM | 4537 | CE | LYS | B | 296 | −9.147 | 3.860 | −31.775 | 1.00 | 51.12 | B | C |
| ATOM | 4538 | NZ | LYS | B | 296 | −7.865 | 4.414 | −31.217 | 1.00 | 50.10 | B | N |
| ATOM | 4539 | C | LYS | B | 296 | −10.477 | −0.407 | −28.291 | 1.00 | 39.86 | B | C |
| ATOM | 4540 | O | LYS | B | 296 | −9.340 | −0.751 | −28.633 | 1.00 | 39.63 | B | O |
| ATOM | 4541 | N | ALA | B | 297 | −11.013 | −0.758 | −27.131 | 1.00 | 38.67 | B | N |
| ATOM | 4542 | CA | ALA | B | 297 | −10.337 | −1.648 | −26.193 | 1.00 | 36.50 | B | C |
| ATOM | 4543 | CB | ALA | B | 297 | −11.132 | −1.753 | −24.896 | 1.00 | 35.52 | B | C |
| ATOM | 4544 | C | ALA | B | 297 | −10.156 | −3.017 | −26.823 | 1.00 | 35.43 | B | C |
| ATOM | 4545 | O | ALA | B | 297 | −9.033 | −3.437 | −27.087 | 1.00 | 35.98 | B | O |
| ATOM | 4546 | N | TYR | B | 298 | −11.266 | −3.697 | −27.098 | 1.00 | 33.26 | B | N |
| ATOM | 4547 | CA | TYR | B | 298 | −11.208 | −5.015 | −27.703 | 1.00 | 30.77 | B | C |
| ATOM | 4548 | CB | TYR | B | 298 | −12.615 | −5.568 | −27.929 | 1.00 | 29.47 | B | C |
| ATOM | 4549 | CG | TYR | B | 298 | −13.204 | −6.108 | −26.644 | 1.00 | 28.38 | B | C |
| ATOM | 4550 | CD1 | TYR | B | 298 | −12.809 | −7.349 | −26.131 | 1.00 | 29.24 | B | C |
| ATOM | 4551 | CE1 | TYR | B | 298 | −13.318 | −7.832 | −24.930 | 1.00 | 28.21 | B | C |
| ATOM | 4552 | CZ | TYR | B | 298 | −14.234 | −7.070 | −24.228 | 1.00 | 27.78 | B | C |
| ATOM | 4553 | OH | TYR | B | 298 | −14.763 | −7.485 | −23.031 | 1.00 | 26.67 | B | O |
| ATOM | 4554 | CE2 | TYR | B | 298 | −14.638 | −5.854 | −24.728 | 1.00 | 27.85 | B | C |
| ATOM | 4555 | CD2 | TYR | B | 298 | −14.119 | −5.381 | −25.925 | 1.00 | 27.58 | B | C |
| ATOM | 4556 | C | TYR | B | 298 | −10.332 | −5.057 | −28.972 | 1.00 | 31.41 | B | C |
| ATOM | 4557 | O | TYR | B | 298 | −9.596 | −6.029 | −29.171 | 1.00 | 33.75 | B | O |
| ATOM | 4558 | N | THR | B | 299 | −10.341 | −4.007 | −29.793 | 1.00 | 30.17 | B | N |
| ATOM | 4559 | CA | THR | B | 299 | −9.452 | −3.990 | −30.981 | 1.00 | 31.63 | B | C |
| ATOM | 4560 | CB | THR | B | 299 | −9.651 | −2.727 | −31.904 | 1.00 | 30.00 | B | C |
| ATOM | 4561 | OG1 | THR | B | 299 | −11.031 | −2.587 | −32.274 | 1.00 | 29.34 | B | O |
| ATOM | 4562 | CG2 | THR | B | 299 | −8.825 | −2.848 | −33.164 | 1.00 | 28.55 | B | C |
| ATOM | 4563 | C | THR | B | 299 | −7.963 | −4.124 | −30.561 | 1.00 | 31.90 | B | C |
| ATOM | 4564 | O | THR | B | 299 | −7.142 | −4.745 | −31.262 | 1.00 | 31.07 | B | O |
| ATOM | 4565 | N | ALA | B | 300 | −7.641 | −3.552 | −29.406 | 1.00 | 32.45 | B | N |
| ATOM | 4566 | CA | ALA | B | 300 | −6.262 | −3.429 | −28.966 | 1.00 | 33.91 | B | C |
| ATOM | 4567 | CB | ALA | B | 300 | −6.157 | −2.425 | −27.831 | 1.00 | 35.00 | B | C |
| ATOM | 4568 | C | ALA | B | 300 | −5.718 | −4.754 | −28.521 | 1.00 | 34.05 | B | C |
| ATOM | 4569 | O | ALA | B | 300 | −4.519 | −5.032 | −28.722 | 1.00 | 33.41 | B | O |
| ATOM | 4570 | N | VAL | B | 301 | −6.606 | −5.551 | −27.916 | 1.00 | 34.40 | B | N |
| ATOM | 4571 | CA | VAL | B | 301 | −6.294 | −6.923 | −27.499 | 1.00 | 35.91 | B | C |
| ATOM | 4572 | CB | VAL | B | 301 | −7.080 | −7.346 | −26.243 | 1.00 | 35.35 | B | C |
| ATOM | 4573 | CG1 | VAL | B | 301 | −6.679 | −6.467 | −25.065 | 1.00 | 34.72 | B | C |
| ATOM | 4574 | CG2 | VAL | B | 301 | −8.587 | −7.313 | −26.485 | 1.00 | 35.39 | B | C |
| ATOM | 4575 | C | VAL | B | 301 | −6.491 | −7.968 | −28.607 | 1.00 | 37.99 | B | C |
| ATOM | 4576 | O | VAL | B | 301 | −6.283 | −9.152 | −28.384 | 1.00 | 40.13 | B | O |
| ATOM | 4577 | N | GLY | B | 302 | −6.875 | −7.535 | −29.801 | 1.00 | 39.20 | B | N |
| ATOM | 4578 | CA | GLY | B | 302 | −6.806 | −8.402 | −30.975 | 1.00 | 38.61 | B | C |
| ATOM | 4579 | C | GLY | B | 302 | −8.120 | −9.085 | −31.268 | 1.00 | 37.68 | B | C |
| ATOM | 4580 | O | GLY | B | 302 | −8.219 | −9.890 | −32.206 | 1.00 | 36.97 | B | O |
| ATOM | 4581 | N | VAL | B | 303 | −9.123 | −8.764 | −30.457 | 1.00 | 36.46 | B | N |
| ATOM | 4582 | CA | VAL | B | 303 | −10.463 | −9.269 | −30.646 | 1.00 | 36.09 | B | C |
| ATOM | 4583 | CB | VAL | B | 303 | −11.186 | −9.418 | −29.310 | 1.00 | 34.30 | B | C |
| ATOM | 4584 | CG1 | VAL | B | 303 | −12.615 | −9.884 | −29.525 | 1.00 | 34.12 | B | C |
| ATOM | 4585 | CG2 | VAL | B | 303 | −10.413 | −10.378 | −28.422 | 1.00 | 34.22 | B | C |
| ATOM | 4586 | C | VAL | B | 303 | −11.210 | −8.285 | −31.529 | 1.00 | 39.41 | B | C |
| ATOM | 4587 | O | VAL | B | 303 | −11.410 | −7.128 | −31.162 | 1.00 | 39.85 | B | O |
| ATOM | 4588 | N | ASN | B | 304 | −11.623 | −8.767 | −32.695 | 1.00 | 42.29 | B | N |
| ATOM | 4589 | CA | ASN | B | 304 | −12.178 | −7.934 | −33.741 | 1.00 | 43.11 | B | C |
| ATOM | 4590 | CB | ASN | B | 304 | −11.400 | −8.162 | −35.039 | 1.00 | 43.44 | B | C |
| ATOM | 4591 | CG | ASN | B | 304 | −9.894 | −7.915 | −34.868 | 1.00 | 44.48 | B | C |
| ATOM | 4592 | OD1 | ASN | B | 304 | −9.046 | −8.788 | −35.169 | 1.00 | 39.07 | B | O |
| ATOM | 4593 | ND2 | ASN | B | 304 | −9.554 | −6.727 | −34.345 | 1.00 | 44.40 | B | N |
| ATOM | 4594 | C | ASN | B | 304 | −13.644 | −8.265 | −33.895 | 1.00 | 45.91 | B | C |
| ATOM | 4595 | O | ASN | B | 304 | −14.115 | −9.329 | −33.478 | 1.00 | 47.21 | B | O |
| ATOM | 4596 | OXT | ASN | B | 304 | −14.398 | −7.442 | −34.401 | 1.00 | 50.28 | B | O |
| HETATM | 4597 | O | HOH | D | 1 | −14.453 | −29.833 | −15.752 | 1.00 | 11.48 | | O |
| HETATM | 4598 | O | HOH | D | 2 | −3.559 | −28.043 | −4.163 | 1.00 | 10.41 | | O |
| HETATM | 4599 | O | HOH | D | 3 | 4.756 | −48.667 | −28.368 | 1.00 | 14.01 | | O |
| HETATM | 4600 | O | HOH | D | 4 | 18.017 | −46.898 | −29.709 | 1.00 | 17.11 | | O |
| HETATM | 4601 | O | HOH | D | 5 | −18.034 | −41.311 | −16.195 | 1.00 | 16.89 | | O |
| HETATM | 4602 | O | HOH | D | 6 | 2.102 | −33.576 | −25.415 | 1.00 | 7.28 | | O |
| HETATM | 4603 | O | HOH | D | 7 | 4.340 | −35.545 | −22.828 | 1.00 | 6.89 | | O |
| HETATM | 4604 | O | HOH | D | 8 | −1.226 | −54.679 | −7.110 | 1.00 | 10.35 | | O |
| HETATM | 4605 | O | HOH | D | 9 | −12.874 | −50.118 | −9.369 | 1.00 | 13.13 | | O |
| HETATM | 4606 | O | HOH | D | 10 | −1.229 | −11.651 | −14.935 | 1.00 | 8.69 | | O |
| HETATM | 4607 | O | HOH | D | 11 | −26.802 | −9.821 | −2.646 | 1.00 | 34.23 | | O |
| HETATM | 4608 | O | HOH | D | 12 | 15.279 | −23.352 | −24.790 | 1.00 | 28.05 | | O |
| HETATM | 4609 | O | HOH | D | 14 | −5.337 | −10.672 | −15.773 | 1.00 | 17.85 | | O |
| HETATM | 4610 | O | HOH | D | 15 | 1.182 | −34.774 | −17.307 | 1.00 | 14.04 | | O |
| HETATM | 4611 | O | HOH | D | 16 | −10.282 | −45.012 | 0.457 | 1.00 | 7.05 | | O |
| HETATM | 4612 | O | HOH | D | 17 | −5.067 | −45.950 | −25.877 | 1.00 | 7.74 | | O |
| HETATM | 4613 | O | HOH | D | 18 | −9.466 | −22.365 | 4.654 | 1.00 | 9.26 | | O |
| HETATM | 4614 | O | HOH | D | 19 | −14.867 | −34.797 | −31.110 | 1.00 | 12.29 | | O |
| HETATM | 4615 | O | HOH | D | 20 | 17.658 | −31.345 | −6.147 | 1.00 | 13.22 | | O |
| HETATM | 4616 | O | HOH | D | 21 | −14.706 | −66.680 | −15.999 | 1.00 | 18.23 | | O |
| HETATM | 4617 | O | HOH | D | 24 | 22.701 | −44.854 | −24.508 | 1.00 | 13.43 | | O |
| HETATM | 4618 | O | HOH | D | 25 | −1.606 | −44.470 | −24.808 | 1.00 | 4.05 | | O |
| HETATM | 4619 | O | HOH | D | 27 | −14.081 | −49.049 | −23.938 | 1.00 | 5.85 | | O |
| HETATM | 4620 | O | HOH | D | 28 | −1.406 | −15.180 | −13.873 | 1.00 | 20.93 | | O |
| HETATM | 4621 | O | HOH | D | 29 | −18.234 | −61.855 | −23.352 | 1.00 | 15.81 | | O |
| HETATM | 4622 | O | HOH | D | 30 | 14.980 | −31.161 | −4.673 | 1.00 | 8.84 | | O |
| HETATM | 4623 | O | HOH | D | 31 | 25.232 | −37.903 | −28.412 | 1.00 | 7.88 | | O |
| HETATM | 4624 | O | HOH | D | 32 | 2.306 | −3.607 | −14.150 | 1.00 | 12.43 | | O |
| HETATM | 4625 | O | HOH | D | 33 | 8.133 | −32.218 | −28.553 | 1.00 | 21.62 | | O |
| HETATM | 4626 | O | HOH | D | 34 | −9.785 | −46.761 | −10.910 | 1.00 | 11.38 | | O |
| HETATM | 4627 | O | HOH | D | 35 | 25.957 | −51.974 | −19.833 | 1.00 | 17.72 | | O |
| HETATM | 4628 | O | HOH | D | 36 | −0.537 | −38.406 | 2.660 | 1.00 | 12.52 | | O |
| HETATM | 4629 | O | HOH | D | 37 | −6.253 | −26.792 | 2.618 | 1.00 | 14.95 | | O |
| HETATM | 4630 | O | HOH | D | 38 | −4.277 | −32.186 | −26.909 | 1.00 | 10.72 | | O |
| HETATM | 4631 | O | HOH | D | 40 | −0.230 | −46.981 | −24.263 | 1.00 | 10.61 | | O |
| HETATM | 4632 | O | HOH | D | 41 | 9.113 | −53.708 | −16.348 | 1.00 | 8.82 | | O |
| HETATM | 4633 | O | HOH | D | 42 | 0.527 | −57.811 | −16.009 | 1.00 | 5.69 | | O |
| HETATM | 4634 | O | HOH | D | 43 | 7.369 | −49.544 | −27.875 | 1.00 | 11.17 | | O |
| HETATM | 4635 | O | HOH | D | 44 | 13.059 | −41.050 | −31.554 | 1.00 | 14.34 | | O |
| HETATM | 4636 | O | HOH | D | 45 | −30.779 | −55.305 | −11.958 | 1.00 | 12.66 | | O |
| HETATM | 4637 | O | HOH | D | 46 | −16.179 | −51.696 | −0.530 | 1.00 | 17.86 | | O |
| HETATM | 4638 | O | HOH | D | 47 | 6.739 | −26.907 | −6.038 | 1.00 | 8.32 | | O |
| HETATM | 4639 | O | HOH | D | 48 | −1.147 | −27.953 | −5.319 | 1.00 | 11.94 | | O |
| HETATM | 4640 | O | HOH | D | 49 | −27.771 | −63.155 | −6.758 | 1.00 | 25.59 | | O |
| HETATM | 4641 | O | HOH | D | 50 | −22.146 | −51.541 | −20.878 | 1.00 | 23.93 | | O |
| HETATM | 4642 | O | HOH | D | 51 | 11.498 | −53.494 | −22.966 | 1.00 | 19.18 | | O |
| HETATM | 4643 | O | HOH | D | 52 | −35.618 | −17.756 | 3.293 | 1.00 | 30.46 | | O |
| HETATM | 4644 | O | HOH | D | 53 | 0.936 | −10.997 | −2.654 | 1.00 | 21.53 | | O |
| HETATM | 4645 | O | HOH | D | 55 | −3.931 | −51.435 | −22.740 | 1.00 | 21.73 | | O |
| HETATM | 4646 | O | HOH | D | 56 | −11.411 | −49.131 | −23.961 | 1.00 | 4.13 | | O |
| HETATM | 4647 | O | HOH | D | 57 | 10.929 | −42.120 | −2.629 | 1.00 | 8.04 | | O |
| HETATM | 4648 | O | HOH | D | 58 | −5.258 | −24.055 | −13.514 | 1.00 | 7.92 | | O |
| HETATM | 4649 | O | HOH | D | 59 | −2.368 | −2.482 | −8.879 | 1.00 | 8.97 | | O |
| HETATM | 4650 | O | HOH | D | 60 | 22.320 | −35.750 | −12.017 | 1.00 | 10.98 | | O |
| HETATM | 4651 | O | HOH | D | 61 | −19.413 | −20.255 | −13.553 | 1.00 | 7.71 | | O |
| HETATM | 4652 | O | HOH | D | 62 | −0.658 | −31.524 | −23.753 | 1.00 | 8.59 | | O |
| HETATM | 4653 | O | HOH | D | 63 | 14.971 | −39.634 | −33.356 | 1.00 | 12.49 | | O |
| HETATM | 4654 | O | HOH | D | 64 | −5.191 | −35.793 | −0.990 | 1.00 | 18.79 | | O |
| HETATM | 4655 | O | HOH | D | 65 | −7.971 | −52.023 | −23.877 | 1.00 | 7.05 | | O |
| HETATM | 4656 | O | HOH | D | 66 | −10.996 | −39.555 | −30.895 | 1.00 | 29.45 | | O |
| HETATM | 4657 | O | HOH | D | 67 | −5.761 | −28.410 | −26.821 | 1.00 | 10.37 | | O |
| HETATM | 4658 | O | HOH | D | 69 | −0.623 | −30.084 | −21.186 | 1.00 | 16.74 | | O |
| HETATM | 4659 | O | HOH | D | 70 | 11.237 | −21.250 | −19.622 | 1.00 | 12.84 | | O |
| HETATM | 4660 | O | HOH | D | 71 | 6.423 | −13.673 | −7.327 | 1.00 | 7.62 | | O |
| HETATM | 4661 | O | HOH | D | 72 | −12.098 | 4.570 | −9.813 | 1.00 | 17.86 | | O |
| HETATM | 4662 | O | HOH | D | 74 | −19.719 | −36.149 | −15.796 | 1.00 | 6.65 | | O |
| HETATM | 4663 | O | HOH | D | 75 | −7.298 | −10.821 | 6.866 | 1.00 | 16.58 | | O |
| HETATM | 4664 | O | HOH | D | 76 | 24.422 | −49.300 | −6.907 | 1.00 | 13.05 | | O |
| HETATM | 4665 | O | HOH | D | 79 | 2.981 | −11.284 | −0.026 | 1.00 | 18.58 | | O |
| HETATM | 4666 | O | HOH | D | 80 | −21.114 | −35.355 | −22.146 | 1.00 | 14.58 | | O |
| HETATM | 4667 | O | HOH | D | 84 | 1.731 | −15.045 | −10.266 | 1.00 | 16.73 | | O |
| HETATM | 4668 | O | HOH | D | 85 | −14.299 | −20.123 | −30.892 | 1.00 | 18.20 | | O |
| HETATM | 4669 | O | HOH | D | 86 | 14.254 | −53.364 | −21.911 | 1.00 | 8.78 | | O |
| HETATM | 4670 | O | HOH | D | 87 | 1.855 | −39.977 | −9.636 | 1.00 | 9.12 | | O |
| HETATM | 4671 | O | HOH | D | 88 | 0.036 | −27.584 | −22.318 | 1.00 | 19.91 | | O |
| HETATM | 4672 | O | HOH | D | 89 | −16.641 | 8.034 | −21.414 | 1.00 | 28.79 | | O |
| HETATM | 4673 | O | HOH | D | 91 | 6.673 | −19.109 | −18.430 | 1.00 | 14.71 | | O |
| HETATM | 4674 | O | HOH | D | 92 | 15.712 | −28.784 | −11.584 | 1.00 | 17.47 | | O |
| HETATM | 4675 | O | HOH | D | 93 | −25.882 | −43.603 | −12.917 | 1.00 | 9.36 | | O |
| HETATM | 4676 | O | HOH | D | 94 | −13.971 | −58.069 | −2.171 | 1.00 | 16.23 | | O |
| HETATM | 4677 | O | HOH | D | 96 | 10.457 | −26.740 | −26.060 | 1.00 | 11.31 | | O |
| HETATM | 4678 | O | HOH | D | 97 | −22.816 | −38.364 | −5.993 | 1.00 | 17.13 | | O |
| HETATM | 4679 | O | HOH | D | 100 | −9.340 | −47.635 | 0.235 | 1.00 | 6.76 | | O |
| HETATM | 4680 | O | HOH | D | 102 | 3.056 | −38.032 | −31.275 | 1.00 | 6.12 | | O |
| HETATM | 4681 | O | HOH | D | 103 | −25.590 | −42.298 | −15.541 | 1.00 | 17.90 | | O |
| HETATM | 4682 | O | HOH | D | 104 | 10.556 | −51.368 | −25.973 | 1.00 | 9.26 | | O |
| HETATM | 4683 | O | HOH | D | 106 | −19.882 | −33.352 | 4.847 | 1.00 | 30.16 | | O |
| HETATM | 4684 | O | HOH | D | 107 | −6.248 | −27.219 | −8.518 | 1.00 | 11.81 | | O |
| HETATM | 4685 | O | HOH | D | 108 | −31.468 | −21.088 | −1.489 | 1.00 | 12.00 | | O |
| HETATM | 4686 | O | HOH | D | 111 | 21.684 | −30.977 | −26.026 | 1.00 | 23.70 | | O |
| HETATM | 4687 | O | HOH | D | 112 | −31.951 | −10.039 | 3.258 | 1.00 | 21.85 | | O |
| HETATM | 4688 | O | HOH | D | 113 | −12.420 | −61.104 | −20.866 | 1.00 | 20.71 | | O |
| HETATM | 4689 | O | HOH | D | 115 | −9.390 | −46.453 | −3.343 | 1.00 | 11.14 | | O |
| HETATM | 4690 | O | HOH | D | 116 | −24.310 | −8.827 | −27.982 | 1.00 | 28.62 | | O |
| HETATM | 4691 | O | HOH | D | 118 | 0.928 | 0.943 | −27.721 | 1.00 | 20.32 | | O |
| HETATM | 4692 | O | HOH | D | 119 | −0.811 | −22.368 | −26.821 | 1.00 | 28.05 | | O |
| HETATM | 4693 | O | HOH | D | 120 | 2.928 | −27.375 | −2.252 | 1.00 | 6.28 | | O |
| HETATM | 4694 | O | HOH | D | 122 | 24.797 | −33.048 | −16.191 | 1.00 | 20.61 | | O |
| HETATM | 4695 | O | HOH | D | 123 | 28.116 | −42.682 | −17.519 | 1.00 | 20.23 | | O |
| HETATM | 4696 | O | HOH | D | 126 | 0.728 | −11.568 | −25.035 | 1.00 | 27.72 | | O |
| HETATM | 4697 | O | HOH | D | 127 | 5.110 | −32.881 | 1.483 | 1.00 | 8.60 | | O |
| HETATM | 4698 | O | HOH | D | 128 | −6.910 | −6.512 | −5.883 | 1.00 | 23.17 | | O |
| HETATM | 4699 | O | HOH | D | 129 | −14.859 | −32.631 | 4.922 | 1.00 | 20.12 | | O |
| HETATM | 4700 | O | HOH | D | 130 | −9.393 | −20.445 | −22.229 | 1.00 | 11.44 | | O |
| HETATM | 4701 | O | HOH | D | 131 | 5.380 | −45.506 | −20.439 | 1.00 | 18.61 | | O |
| HETATM | 4702 | O | HOH | D | 132 | −20.298 | −17.857 | 4.369 | 1.00 | 24.71 | | O |
| HETATM | 4703 | O | HOH | D | 133 | −13.074 | −15.017 | −30.254 | 1.00 | 22.69 | | O |
| HETATM | 4704 | O | HOH | D | 134 | −22.544 | −64.671 | −13.497 | 1.00 | 18.42 | | O |
| HETATM | 4705 | O | HOH | D | 135 | −4.355 | −31.730 | −19.579 | 1.00 | 30.49 | | O |
| HETATM | 4706 | O | HOH | D | 136 | −4.978 | −28.238 | −15.122 | 1.00 | 22.36 | | O |
| HETATM | 4707 | O | HOH | D | 137 | −7.834 | −20.152 | 9.638 | 1.00 | 18.69 | | O |
| HETATM | 4708 | O | HOH | D | 138 | −14.293 | −28.356 | −18.330 | 1.00 | 27.34 | | O |
| HETATM | 4709 | O | HOH | D | 139 | −22.099 | −32.349 | −8.846 | 1.00 | 29.42 | | O |
| HETATM | 4710 | O | HOH | D | 140 | 8.752 | −28.130 | −6.857 | 1.00 | 8.09 | | O |
| HETATM | 4711 | O | HOH | D | 141 | 15.481 | −31.534 | −31.496 | 1.00 | 7.89 | | O |
| HETATM | 4712 | O | HOH | D | 142 | −14.386 | −25.883 | −11.379 | 1.00 | 24.39 | | O |
| HETATM | 4713 | O | HOH | D | 143 | −2.224 | −28.207 | −27.995 | 1.00 | 21.76 | | O |
| HETATM | 4714 | O | HOH | D | 144 | −3.038 | 2.948 | −18.802 | 1.00 | 17.17 | | O |
| HETATM | 4715 | O | HOH | D | 145 | −17.234 | −8.824 | −32.860 | 1.00 | 22.34 | | O |
| HETATM | 4716 | O | HOH | D | 146 | −21.972 | −36.386 | −17.229 | 1.00 | 15.20 | | O |
| HETATM | 4717 | O | HOH | D | 147 | 21.738 | −50.115 | −22.044 | 1.00 | 41.96 | | O |
| HETATM | 4718 | O | HOH | D | 148 | −15.129 | −42.497 | −17.235 | 1.00 | 64.58 | | O |
| HETATM | 4719 | O | HOH | D | 149 | 0.499 | −13.659 | 1.071 | 1.00 | 20.44 | | O |
| HETATM | 4720 | O | HOH | D | 150 | 26.656 | −39.916 | −12.524 | 1.00 | 28.86 | | O |
| HETATM | 4721 | O | HOH | D | 151 | −26.068 | −18.778 | 22.468 | 1.00 | 63.91 | | O |
| HETATM | 4722 | O | HOH | D | 152 | 17.839 | −37.146 | −6.365 | 1.00 | 17.05 | | O |
| HETATM | 4723 | O | HOH | D | 153 | −9.872 | −37.859 | 3.756 | 1.00 | 29.86 | | O |
| HETATM | 4724 | O | HOH | D | 154 | −26.704 | −13.656 | −20.767 | 1.00 | 24.94 | | O |
| HETATM | 4725 | O | HOH | D | 155 | −16.594 | −46.356 | 1.735 | 1.00 | 9.02 | | O |
| HETATM | 4726 | O | HOH | D | 156 | −3.278 | −28.231 | −11.565 | 1.00 | 15.79 | | O |
| HETATM | 4727 | O | HOH | D | 157 | −13.773 | −34.807 | 7.967 | 1.00 | 26.45 | | O |
| HETATM | 4728 | O | HOH | D | 158 | 5.274 | −27.734 | −3.607 | 1.00 | 14.35 | | O |
| HETATM | 4729 | O | HOH | D | 159 | −10.955 | −62.644 | −9.650 | 1.00 | 22.81 | | O |
| HETATM | 4730 | O | HOH | D | 160 | 19.801 | −28.085 | −15.802 | 1.00 | 15.92 | | O |
| HETATM | 4731 | O | HOH | D | 161 | 14.910 | −35.916 | −3.899 | 1.00 | 14.53 | | O |
| HETATM | 4732 | O | HOH | D | 162 | −7.630 | −39.335 | −29.693 | 1.00 | 19.10 | | O |
| HETATM | 4733 | O | HOH | D | 163 | 8.909 | −8.381 | −2.714 | 1.00 | 31.11 | | O |
| HETATM | 4734 | O | HOH | D | 164 | −35.136 | −13.839 | −9.366 | 1.00 | 19.26 | | O |
| HETATM | 4735 | O | HOH | D | 165 | −28.655 | −13.870 | 18.003 | 1.00 | 25.79 | | O |
| HETATM | 4736 | O | HOH | D | 166 | −4.969 | −28.077 | −6.380 | 1.00 | 15.01 | | O |
| HETATM | 4737 | O | HOH | D | 167 | −12.416 | −58.656 | −21.906 | 1.00 | 38.53 | | O |
| HETATM | 4738 | O | HOH | D | 168 | −1.381 | −22.000 | 5.296 | 1.00 | 15.16 | | O |
| HETATM | 4739 | O | HOH | D | 169 | −28.073 | −14.871 | −23.335 | 1.00 | 29.90 | | O |
| HETATM | 4740 | O | HOH | D | 170 | −5.698 | −52.447 | −27.051 | 1.00 | 46.81 | | O |
| HETATM | 4741 | O | HOH | D | 171 | 25.098 | −40.710 | −27.602 | 1.00 | 31.23 | | O |
| HETATM | 4742 | O | HOH | D | 172 | 13.253 | −30.057 | −31.043 | 1.00 | 25.08 | | O |
| HETATM | 4743 | CA | CA | C | 1 | −3.996 | −44.763 | −24.393 | 1.00 | 14.92 | | CA |
| HETATM | 4744 | CA | CA | C | 2 | −9.298 | −38.014 | −30.827 | 1.00 | 19.13 | | CA |
| HETATM | 4745 | CA | CA | C | 3 | −7.033 | −7.984 | −3.851 | 1.00 | 33.23 | | CA |
| HETATM | 4746 | CA | CA | C | 4 | 3.009 | −11.945 | −2.235 | 1.00 | 23.81 | | CA |
| ATOM | 4747 | N | VAL | E | 1 | −6.805 | −32.986 | −18.277 | 1.00 | 18.96 | | N |
| ATOM | 4748 | CA | VAL | E | 1 | −5.543 | −32.961 | −17.454 | 1.00 | 19.68 | | C |
| ATOM | 4749 | CB | VAL | E | 1 | −5.490 | −31.747 | −16.442 | 1.00 | 19.24 | | C |
| ATOM | 4750 | CG1 | VAL | E | 1 | −5.729 | −30.417 | −17.143 | 1.00 | 19.12 | | C |
| ATOM | 4751 | CG2 | VAL | E | 1 | −6.448 | −31.893 | −15.256 | 1.00 | 18.78 | | C |
| ATOM | 4752 | C | VAL | E | 1 | −5.209 | −34.318 | −16.766 | 1.00 | 20.53 | | C |
| ATOM | 4753 | O | VAL | E | 1 | −4.162 | −34.931 | −17.045 | 1.00 | 19.81 | | O |
| ATOM | 4754 | N | ARG | E | 2 | −6.036 | −34.659 | −15.814 | 1.00 | 22.30 | | N |
| ATOM | 4755 | CA | ARG | E | 2 | −5.893 | −35.826 | −14.996 | 1.00 | 22.98 | | C |
| ATOM | 4756 | C | ARG | E | 2 | −5.143 | −35.563 | −13.709 | 1.00 | 23.10 | | C |
| ATOM | 4757 | O | ARG | E | 2 | −3.987 | −35.675 | −13.626 | 1.00 | 21.38 | | O |
| ATOM | 4758 | CB | ARG | E | 2 | −5.370 | −36.923 | −15.862 | 1.00 | 20.00 | | C |
| ATOM | 4759 | CG | ARG | E | 2 | −6.510 | −37.666 | −16.472 | 1.00 | 20.00 | | C |
| ATOM | 4760 | CD | ARG | E | 2 | −6.236 | −37.842 | −17.914 | 1.00 | 20.00 | | C |
| ATOM | 4761 | NE | ARG | E | 2 | −5.023 | −38.606 | −18.152 | 1.00 | 20.00 | | N |
| ATOM | 4762 | CZ | ARG | E | 2 | −4.700 | −39.033 | −19.365 | 1.00 | 20.00 | | C |
| ATOM | 4763 | NH1 | ARG | E | 2 | −5.498 | −38.765 | −20.370 | 1.00 | 20.00 | | N |
| ATOM | 4764 | NH2 | ARG | E | 2 | −3.590 | −39.683 | −19.606 | 1.00 | 20.00 | | N |
| ATOM | 4765 | N | ALA | E | 3 | −5.874 | −35.228 | −12.682 | 1.00 | 25.76 | | N |
| ATOM | 4766 | CA | ALA | E | 3 | −5.370 | −34.410 | −11.547 | 1.00 | 29.63 | | C |
| ATOM | 4767 | CB | ALA | E | 3 | −6.254 | −33.159 | −11.469 | 1.00 | 30.22 | | C |
| ATOM | 4768 | C | ALA | E | 3 | −5.257 | −35.094 | −10.105 | 1.00 | 31.58 | | C |
| ATOM | 4769 | O | ALA | E | 3 | −5.874 | −36.130 | −9.844 | 1.00 | 31.51 | | O |
| ATOM | 4770 | N | ALA | E | 4 | −4.491 | −34.497 | −9.169 | 1.00 | 32.55 | | N |
| ATOM | 4771 | CA | ALA | E | 4 | −4.349 | −35.033 | −7.771 | 1.00 | 30.23 | | C |
| ATOM | 4772 | CB | ALA | E | 4 | −3.660 | −36.382 | −7.798 | 1.00 | 28.96 | | C |
| ATOM | 4773 | C | ALA | E | 4 | −3.600 | −34.141 | −6.772 | 1.00 | 29.50 | | C |
| ATOM | 4774 | O | ALA | E | 4 | −2.820 | −34.655 | −5.948 | 1.00 | 27.58 | | O |
| ATOM | 4775 | N | ARG | F | 1 | −8.429 | −19.552 | −9.416 | 1.00 | 25.91 | | N |
| ATOM | 4776 | CA | ARG | F | 1 | −9.556 | −20.144 | −8.678 | 1.00 | 28.61 | | C |
| ATOM | 4777 | C | ARG | F | 1 | −10.743 | −20.675 | −9.479 | 1.00 | 29.09 | | C |
| ATOM | 4778 | O | ARG | F | 1 | −11.224 | −20.098 | −10.395 | 1.00 | 27.32 | | O |
| ATOM | 4779 | CB | ARG | F | 1 | −10.034 | −19.244 | −7.562 | 1.00 | 20.00 | | C |
| ATOM | 4780 | CG | ARG | F | 1 | −9.026 | −18.241 | −7.099 | 1.00 | 20.00 | | C |
| ATOM | 4781 | CD | ARG | F | 1 | −9.684 | −16.875 | −6.961 | 1.00 | 20.00 | | C |
| ATOM | 4782 | NE | ARG | F | 1 | −8.851 | −15.706 | −6.762 | 1.00 | 20.00 | | N |
| ATOM | 4783 | CZ | ARG | F | 1 | −7.599 | −15.716 | −6.317 | 1.00 | 20.00 | | C |
| ATOM | 4784 | NH1 | ARG | F | 1 | −6.959 | −16.845 | −6.043 | 1.00 | 20.00 | | N |
| ATOM | 4785 | NH2 | ARG | F | 1 | −6.952 | −14.572 | −6.163 | 1.00 | 20.00 | | N |
| ATOM | 4786 | N | ALA | F | 2 | −11.202 | −21.816 | −9.078 | 1.00 | 31.68 | | N |
| ATOM | 4787 | CA | ALA | F | 2 | −11.921 | −22.706 | −9.931 | 1.00 | 35.91 | | C |
| ATOM | 4788 | CB | ALA | F | 2 | −11.159 | −24.023 | −9.929 | 1.00 | 36.85 | | C |
| ATOM | 4789 | C | ALA | F | 2 | −13.430 | −22.961 | −9.726 | 1.00 | 38.32 | | C |
| ATOM | 4790 | O | ALA | F | 2 | −13.892 | −23.333 | −8.669 | 1.00 | 34.79 | | O |
| ATOM | 4791 | N | ALA | F | 3 | −14.167 | −22.664 | −10.769 | 1.00 | 40.70 | | N |
| ATOM | 4792 | CA | ALA | F | 3 | −15.085 | −23.497 | −11.515 | 1.00 | 40.16 | | C |
| ATOM | 4793 | CB | ALA | F | 3 | −14.939 | −24.977 | −11.187 | 1.00 | 40.29 | | C |
| ATOM | 4794 | C | ALA | F | 3 | −16.524 | −23.097 | −11.612 | 1.00 | 37.96 | | C |
| ATOM | 4795 | O | ALA | F | 3 | −17.129 | −23.397 | −12.612 | 1.00 | 35.80 | | O |
| END | | | | | | | | | | | | |

### Example 3

### Comparison of PehPro1 with Thermolysin Structure

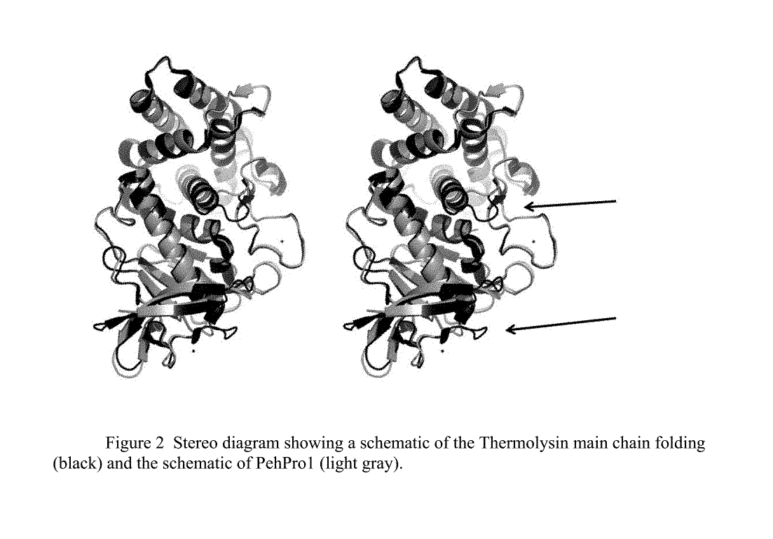

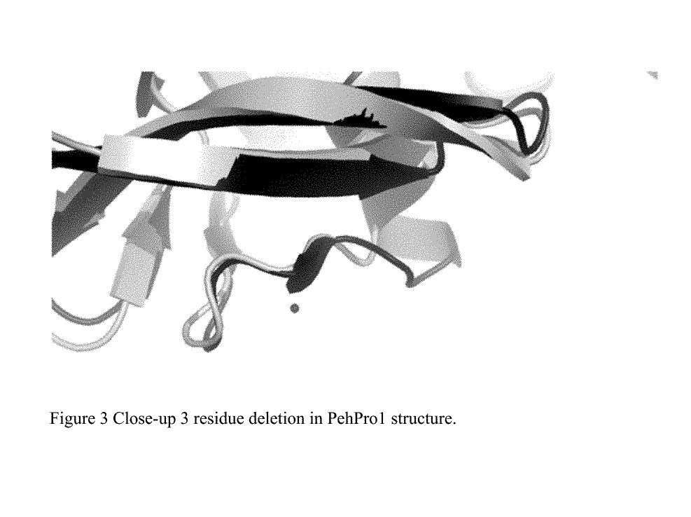

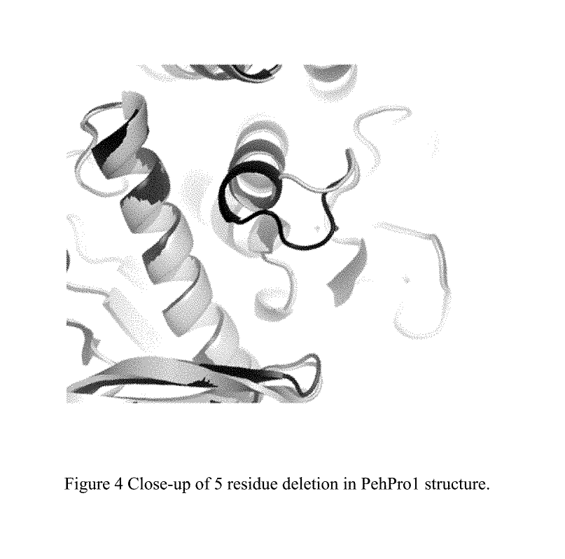

The structure of PehPro1 was compared to that of Thermolysin (B. thermoproteolyticus metalloprotease, pdb 1KEI.A) [Senda, M., Senda, T. and Kidokoro, S., Crystal Structure Analyses Of Thermolysin In Complex With Its Inhibitors, Direct Submission]. The overall folding of PehPro1 is highly similar to Thermolysin and other known metalloproteases from Bacillus (B. cereus (pdb 1NPC.A) [Sidler, W., Niederer, E., Suter, F. and Zuber, H., The primary structure of Bacillus cereus neutral proteinase and comparison with thermolysin and Bacillus subtilis neutral Proteinase, Biol. Chem. Hoppe-Seyler 367 (7), 643-657 (1986)] and B. stearothermophilus, and B. subtilis metalloproteases, consisting of two domains and a central connecting helix. A schematic of the overall topology of PehPro1 is presented and compared with Thermolysin in FIG. 2. The main differences were found in regions where there were deletions in the PehPro1 sequence relative to that of other known metalloprotease structures. Two regions of deletion occur in PehPro1, and as a consequence the main chain folding deviates from thermolysin after residue Trp58 of PehPro1 (see FIG. 3), and after residue Asp170 of PehPro1 (see FIG. 4). Four residues in Thermolysin are replaced by Asn59 in PehPro1 and seven residues in Thermolysin are replaced by Gly171-Lys172 in PehPro1, these deletions are indicated by arrows in FIG. 2. The numbering of residues corresponds to the linear contiguous sequence of each mature enzyme, respectively.

Four calcium ions are bound in the Thermolysin structure: two at a double cation site (Ca1,2), and one in each of the single cation sites (Ca3, Ca4). In contrast to Thermolysin, the PehPro1 structure has only two calcium binding sites, a single one near the double cation site (Ca1-2) in Thermolysin and a second (Ca4) that seems to be conserved in the two molecules. One Thermolysin calcium site (Ca3) is completely absent in the PehPro1 structure. Since calcium dependence is considered to be a factor in some potential uses of these proteases, particularly as a detergent additive where builders are present specifically to reduce the hardness of water by chelating ions such as calcium and magnesium, these enzymes may prove to have reduced cation sensitivity and hence improved stability under conditions of low calcium availability.

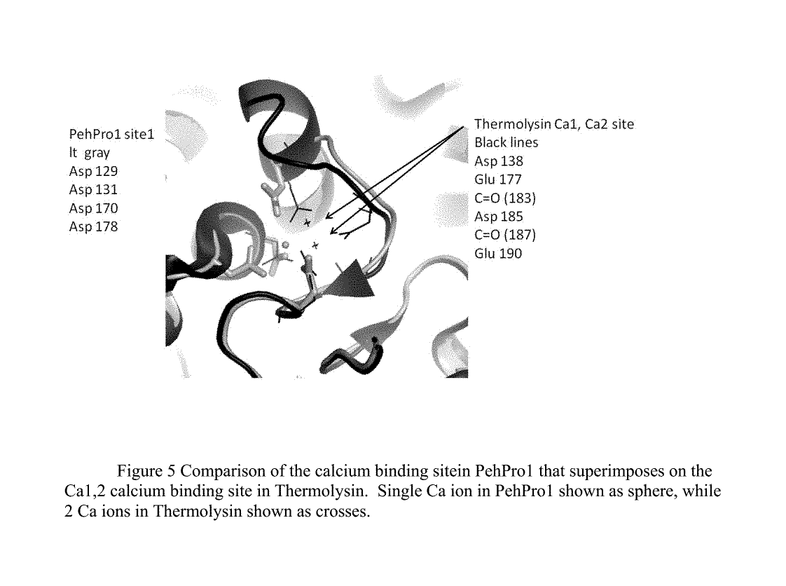

The region around the Thermolysin double cation site (Ca1,2) is shown in FIG. 5. In this figure, the structure of Thermolysin is present as black lines, with the two calcium ions shown as crosses. The superimposed structure of PehPro1 is shown as a stick figure with its single calcium ion as a non-bonding sphere. It may be seen that the two sites are substantially different. In Thermolysin, six residues along with solvent are present to stabilize the ion pair, including side chains of Asp138, Glu177, Asp 185, Glu190, and the main chain carbonyls of residues Asn183 and Glu187 (Thermolysin numbering). In PehPro1 (numbering relative to mature PehPro1), the calcium in the same vicinity is stabilized by side chains of Asp129, Asp131, Asp170 and Asp178 along with the solvent. Only Asp131 and Asp178 in PehPro1 are homologous with side chains in Thermolysin Asp138 and Glu190, respectively.

The second calcium binding site in PehPro1 is compared with the homologous site (Ca4) in Thermolysin in FIG. 6. In this instance, there is a one to one correspondence of residues forming the calcium binding site in both Thermolysin and PehPro1. In Thermolysin, the side chains of Thr194 and Asp200 (Thermolysin numbering) along with the main chain carbonyls of residues Tyr193, Thr194 and Ile197 form ligands to this calcium ion, while in PehPro1 it is the homologous residues Thr182 and Asp188 along with the carbonyl oxygen of residues Tyr181, Thr182 and Thr185 (numbering relative to mature PehPro1).

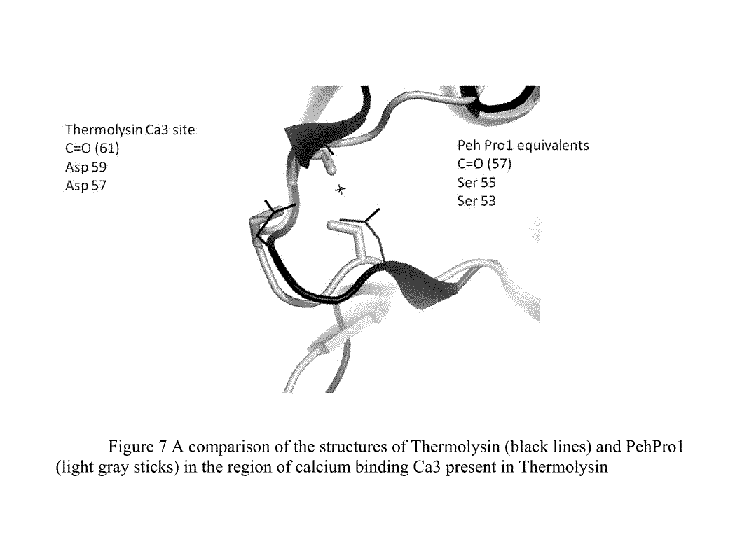

The structures of PehPro1 and Thermolysin are compared in the vicinity of the Ca3 calcium site in Thermolysin in FIG. 7. In Thermolysin the Ca3 calcium site is formed in a loop containing two aspartic acids residues Asp57 and Asp59, which along with the main chain carbonyl oxygen and solvent form ligands to the calcium ion, In contrast, in PehPro1, the two (Asp) ligands are replaced with serine residues which, while in homologous conformations, will not stabilize binding of a calcium ion. In the electron density map there is no evidence of calcium binding under the conditions of crystallization.

### Example 4

### Comparison of Structures of PehPro1 and PpoPro2 Metalloproteases

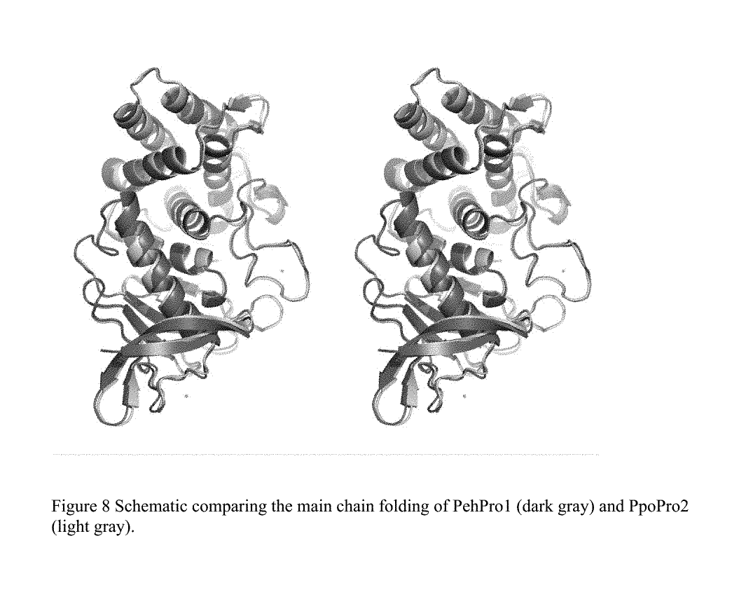

Recently, the structure of a metalloprotease from a member of the Paenibacillus genus, Paenibacillus polymyxa (PpoPro2), was reported (Ruf et al, Acta Cryst. D 69, 24-31 (2013)). The overall folding of PpoPro2 is highly homologous to Thermolysin and other known M4 metalloproteases. The PehPro1 and PpoPro2 structures consist of 304 residues that are aligned without insertions or deletions relative to each other. The PpoPro2 and PehPro1 structures therefore share a common pattern of deletions relative to that of other known metalloprotease structures. The overall folding of the PehPro1 and PpoPro2 is presented in FIG. 8.

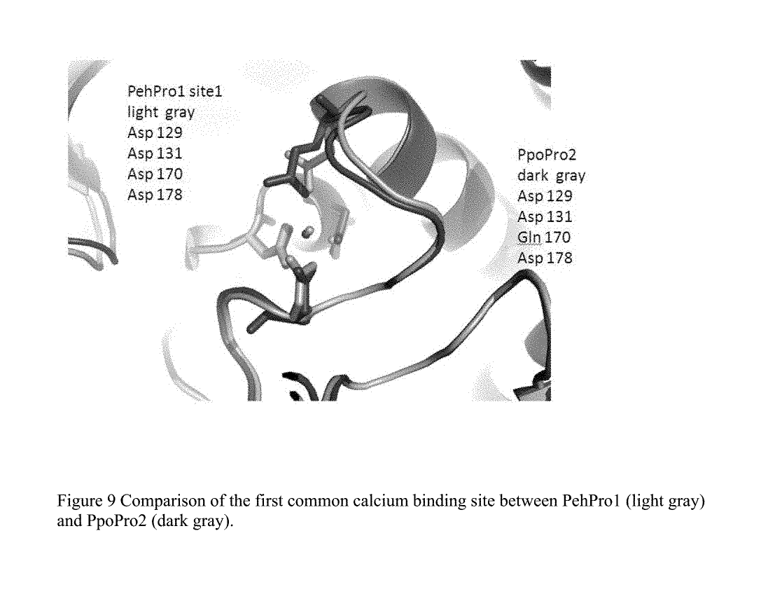

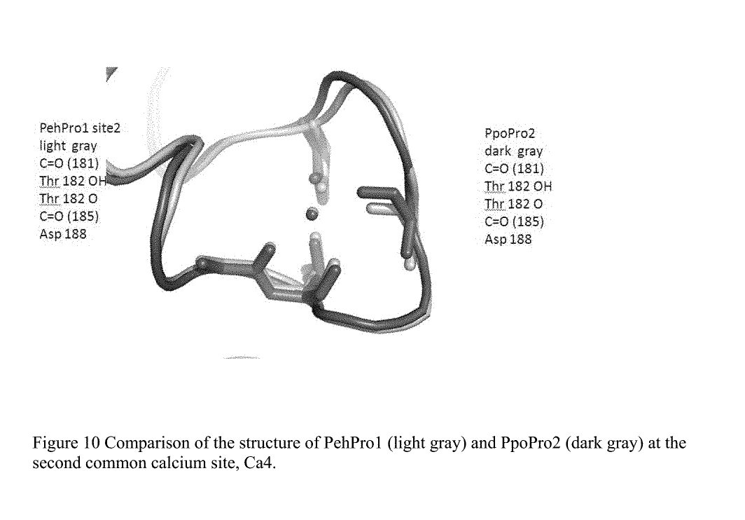

In contrast to PehPro1, the structure of PpoPro2 was found to bind three calcium ions. The first two (Ca4 and a variant of Ca1-2) described below are highly homologous between PehPro1 and PpoPro2 molecules, and the third is homologous to calcium site Ca3 site. The common sites are compared in FIGS. 9 and 10. In both sites, all interactions seen in either PehPro1 or PpoPro2 are conserved in the other.

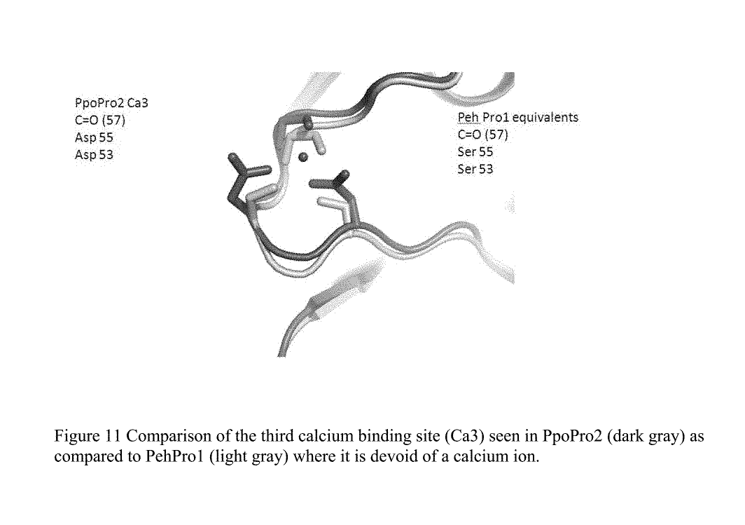

As mentioned above, the PpoPro2 has an additional calcium ion bound at Ca3 that is not seen in PehPro1. Just as in the structure of Thermolysin (FIG. 7), aspartic acid residues are found in PpoPro2 whereas in PehPro1, we find Ser 53 and Ser55 instead of the aspartic acid residues (FIG. 11).

### Example 5

### Crystallization and Structure Determination of NprE

The metalloprotease NprE, obtained from Bacillus subtilis, is known to perform in detergent formulations (as described in U.S. Pat. No. 8,114,656 B2 Shaw et al and others). An NprE variant (S129I/F130L/M138L/V190I/D220P) was crystallized using the hanging drop method from a solution of protein stock at a concentration of 26.2 mg/mL in 40% Propylene Glycol+50 mM IVIES pH 5.4+1 mM Calcium chloride. Aliquots of 3 μL of the protein stock and 3 μL of the crystallization solution were mixed on a plastic coverslip and inverted and sealed on a chamber containing 23% Polyethylene Glycol 4000+0.20M Lithium chloride+0.09M Bis Tris Propane pH 6.5+12% Isopropanol+4 mM Zinc chloride+12.5 mM Yttrium chloride in a Linbro 6×4 culture plate.

Crystals grew in the hexagonal space group P6(3)22 with unit cell dimensions; a=122.9 Å, b=122.9 Å, and c=119.1 Å. Data were collected on native crystal to 2.5 Å and the structure of NprE was determined by molecular replacement using a related protein (pdb ID 1ESP) as the phasing model. The statistics of data collection are presented in Table 5.1.

TABLE 5.1
Statistics of NprE Data collection

| Wavelength | 1.54 | Å |
|---|---|---|
| Space group | P6322 | |
| Molecules in asymmetric unit | 1 | |
| Unit cell dimensions | a = 122.9 Å, b = 122.9 Å and | |
| | c = 119.1 Å | |
| Resolution | 30.0-2.49 | Å |
| Unique reflections | 17969 | |
| Multiplicity | 7.46 | (7.36*) |
| Completeness | 99.94% | (99.9*) |
| R_{merge} | 0.067 | (0.25*) |
| I/σ_{I} | 17.2 | (5.9*) |

*Value in parenthesis is that of the outermost shell of data

The model was fitted in the resulting electron density using the program COOT [Emsley, P et al (2010) Acta Cryst. D66 486-501]. After fitting and refitting adjustments, the coordinates were refined using the REFMAC program with standard defaults in the CCP4 software suite. The statistics of the current model are presented in Table 5.2.

TABLE 5.2
Statistics of the refined model

| | |
|---|---|
| R work | 0.20 |
| R free | 0.23 |
| No. protein residues | 300 |
| No. atoms | 2444 |
| rmsd Bond lengths | 0.024 Å |
| rmsd bond angles | 2.1° |

The coordinates for structure of the NprE variant are provided below.

| | | | | | | | | | | | | |
|---|---|---|---|---|---|---|---|---|---|---|---|---|
| ATOM | 1 | N | ALA | A | 1 | −50.837 | −39.587 | −13.317 | 1.00 | 46.97 | A | N |
| ATOM | 2 | CA | ALA | A | 1 | −51.606 | −39.753 | −14.634 | 1.00 | 45.97 | A | C |
| ATOM | 3 | CB | ALA | A | 1 | −52.426 | −38.479 | −14.961 | 1.00 | 46.00 | A | C |
| ATOM | 4 | C | ALA | A | 1 | −50.598 | −40.107 | −15.764 | 1.00 | 45.46 | A | C |
| ATOM | 5 | O | ALA | A | 1 | −49.808 | −39.271 | −16.244 | 1.00 | 43.56 | A | O |
| ATOM | 6 | N | ALA | A | 2 | −50.582 | −41.387 | −16.118 | 1.00 | 45.03 | A | N |
| ATOM | 7 | CA | ALA | A | 2 | −49.511 | −41.916 | −16.936 | 1.00 | 44.55 | A | C |
| ATOM | 8 | CB | ALA | A | 2 | −49.635 | −43.447 | −17.040 | 1.00 | 44.73 | A | C |
| ATOM | 9 | C | ALA | A | 2 | −49.578 | −41.279 | −18.307 | 1.00 | 45.19 | A | C |
| ATOM | 10 | O | ALA | A | 2 | −50.549 | −41.460 | −19.038 | 1.00 | 46.01 | A | O |
| ATOM | 11 | N | THR | A | 3 | −48.571 | −40.506 | −18.675 | 1.00 | 45.24 | A | N |
| ATOM | 12 | CA | THR | A | 3 | −48.541 | −40.003 | −20.048 | 1.00 | 44.84 | A | C |
| ATOM | 13 | CB | THR | A | 3 | −49.272 | −38.639 | −20.200 | 1.00 | 44.49 | A | C |
| ATOM | 14 | OG1 | THR | A | 3 | −49.432 | −38.341 | −21.588 | 1.00 | 42.12 | A | O |
| ATOM | 15 | CG2 | THR | A | 3 | −48.529 | −37.520 | −19.521 | 1.00 | 41.95 | A | C |
| ATOM | 16 | C | THR | A | 3 | −47.144 | −40.023 | −20.726 | 1.00 | 45.42 | A | C |
| ATOM | 17 | O | THR | A | 3 | −46.153 | −40.548 | −20.182 | 1.00 | 46.03 | A | O |
| ATOM | 18 | N | THR | A | 4 | −47.105 | −39.436 | −21.913 | 1.00 | 44.79 | A | N |
| ATOM | 19 | CA | THR | A | 4 | −45.981 | −39.502 | −22.803 | 1.00 | 44.31 | A | C |
| ATOM | 20 | CB | THR | A | 4 | −46.352 | −40.377 | −24.023 | 1.00 | 45.17 | A | C |
| ATOM | 21 | OG1 | THR | A | 4 | −45.615 | −41.611 | −23.914 | 1.00 | 45.01 | A | O |
| ATOM | 22 | CG2 | THR | A | 4 | −46.117 | −39.684 | −25.418 | 1.00 | 46.18 | A | C |
| ATOM | 23 | C | THR | A | 4 | −45.505 | −38.090 | −23.095 | 1.00 | 43.59 | A | C |
| ATOM | 24 | O | THR | A | 4 | −46.263 | −37.144 | −22.990 | 1.00 | 43.13 | A | O |
| ATOM | 25 | N | GLY | A | 5 | −44.220 | −37.949 | −23.385 | 1.00 | 42.62 | A | N |
| ATOM | 26 | CA | GLY | A | 5 | −43.593 | −36.642 | −23.465 | 1.00 | 40.86 | A | C |
| ATOM | 27 | C | GLY | A | 5 | −42.380 | −36.751 | −24.358 | 1.00 | 39.27 | A | C |
| ATOM | 28 | O | GLY | A | 5 | −42.184 | −37.762 | −25.008 | 1.00 | 39.11 | A | O |
| ATOM | 29 | N | THR | A | 6 | −41.553 | −35.717 | −24.347 | 1.00 | 38.05 | A | N |
| ATOM | 30 | CA | THR | A | 6 | −40.412 | −35.657 | −25.213 | 1.00 | 37.20 | A | C |
| ATOM | 31 | CB | THR | A | 6 | −40.907 | −35.169 | −26.624 | 1.00 | 37.93 | A | C |
| ATOM | 32 | OG1 | THR | A | 6 | −40.731 | −36.218 | −27.585 | 1.00 | 37.23 | A | O |
| ATOM | 33 | CG2 | THR | A | 6 | −40.272 | −33.892 | −27.082 | 1.00 | 37.05 | A | C |
| ATOM | 34 | C | THR | A | 6 | −39.401 | −34.748 | −24.523 | 1.00 | 36.18 | A | C |
| ATOM | 35 | O | THR | A | 6 | −39.794 | −33.933 | −23.690 | 1.00 | 36.69 | A | O |
| ATOM | 36 | N | GLY | A | 7 | −38.109 | −34.926 | −24.791 | 1.00 | 34.95 | A | N |
| ATOM | 37 | CA | GLY | A | 7 | −37.092 | −34.018 | −24.258 | 1.00 | 33.82 | A | C |
| ATOM | 38 | C | GLY | A | 7 | −35.792 | −34.134 | −25.024 | 1.00 | 33.59 | A | C |
| ATOM | 39 | O | GLY | A | 7 | −35.645 | −35.006 | −25.862 | 1.00 | 34.18 | A | O |
| ATOM | 40 | N | THR | A | 8 | −34.849 | −33.253 | −24.732 | 1.00 | 33.90 | A | N |
| ATOM | 41 | CA | THR | A | 8 | −33.598 | −33.129 | −25.464 | 1.00 | 34.22 | A | C |
| ATOM | 42 | CB | THR | A | 8 | −33.226 | −31.608 | −25.718 | 1.00 | 34.37 | A | C |
| ATOM | 43 | OG1 | THR | A | 8 | −34.299 | −30.920 | −26.401 | 1.00 | 36.58 | A | O |
| ATOM | 44 | CG2 | THR | A | 8 | −31.947 | −31.481 | −26.536 | 1.00 | 33.08 | A | C |
| ATOM | 45 | C | THR | A | 8 | −32.453 | −33.790 | −24.669 | 1.00 | 34.72 | A | C |
| ATOM | 46 | O | THR | A | 8 | −32.184 | −33.426 | −23.489 | 1.00 | 34.25 | A | O |
| ATOM | 47 | N | THR | A | 9 | −31.753 | −34.728 | −25.323 | 1.00 | 34.59 | A | N |
| ATOM | 48 | CA | THR | A | 9 | −30.639 | −35.395 | −24.677 | 1.00 | 34.66 | A | C |
| ATOM | 49 | CB | THR | A | 9 | −30.238 | −36.722 | −25.329 | 1.00 | 33.98 | A | C |
| ATOM | 50 | OG1 | THR | A | 9 | −29.590 | −36.445 | −26.559 | 1.00 | 37.28 | A | O |
| ATOM | 51 | CG2 | THR | A | 9 | −31.414 | −37.551 | −25.590 | 1.00 | 33.59 | A | C |
| ATOM | 52 | C | THR | A | 9 | −29.431 | −34.488 | −24.559 | 1.00 | 34.50 | A | C |
| ATOM | 53 | O | THR | A | 9 | −29.385 | −33.400 | −25.144 | 1.00 | 33.46 | A | O |
| ATOM | 54 | N | LEU | A | 10 | −28.479 | −34.947 | −23.749 | 1.00 | 34.53 | A | N |
| ATOM | 55 | CA | LEU | A | 10 | −27.257 | −34.224 | −23.481 | 1.00 | 35.27 | A | C |
| ATOM | 56 | CB | LEU | A | 10 | −26.367 | −35.131 | −22.654 | 1.00 | 34.24 | A | C |
| ATOM | 57 | CG | LEU | A | 10 | −25.892 | −34.808 | −21.256 | 1.00 | 33.07 | A | C |
| ATOM | 58 | CD1 | LEU | A | 10 | −26.322 | −33.431 | −20.727 | 1.00 | 35.04 | A | C |
| ATOM | 59 | CD2 | LEU | A | 10 | −26.213 | −35.900 | −20.296 | 1.00 | 29.81 | A | C |
| ATOM | 60 | C | LEU | A | 10 | −26.539 | −33.859 | −24.786 | 1.00 | 37.02 | A | C |
| ATOM | 61 | O | LEU | A | 10 | −25.854 | −32.837 | −24.839 | 1.00 | 36.69 | A | O |
| ATOM | 62 | N | LYS | A | 11 | −26.672 | −34.715 | −25.801 | 1.00 | 38.07 | A | N |
| ATOM | 63 | CA | LYS | A | 11 | −26.036 | −34.498 | −27.084 | 1.00 | 41.05 | A | C |
| ATOM | 64 | CB | LYS | A | 11 | −25.488 | −35.850 | −27.666 | 1.00 | 41.74 | A | C |
| ATOM | 65 | CG | LYS | A | 11 | −24.330 | −36.532 | −26.896 | 1.00 | 41.97 | A | C |
| ATOM | 66 | CD | LYS | A | 11 | −23.014 | −35.884 | −27.186 | 1.00 | 40.92 | A | C |
| ATOM | 67 | CE | LYS | A | 11 | −21.854 | −36.797 | −26.839 | 1.00 | 44.19 | A | C |
| ATOM | 68 | NZ | LYS | A | 11 | −21.535 | −36.808 | −25.375 | 1.00 | 43.03 | A | N |
| ATOM | 69 | C | LYS | A | 11 | −26.996 | −33.861 | −28.134 | 1.00 | 42.61 | A | C |
| ATOM | 70 | O | LYS | A | 11 | −26.661 | −33.822 | −29.329 | 1.00 | 43.21 | A | O |
| ATOM | 71 | N | GLY | A | 12 | −28.198 | −33.427 | −27.730 | 1.00 | 43.01 | A | N |
| ATOM | 72 | CA | GLY | A | 12 | −29.046 | −32.651 | −28.623 | 1.00 | 42.11 | A | C |
| ATOM | 73 | C | GLY | A | 12 | −30.002 | −33.454 | −29.460 | 1.00 | 42.80 | A | C |
| ATOM | 74 | O | GLY | A | 12 | −30.566 | −32.919 | −30.409 | 1.00 | 43.22 | A | O |
| ATOM | 75 | N | LYS | A | 13 | −30.208 | −34.725 | −29.127 | 1.00 | 43.45 | A | N |
| ATOM | 76 | CA | LYS | A | 13 | −31.271 | −35.553 | −29.763 | 1.00 | 44.11 | A | C |
| ATOM | 77 | CB | LYS | A | 13 | −30.888 | −37.042 | −29.814 | 1.00 | 44.72 | A | C |
| ATOM | 78 | CG | LYS | A | 13 | −29.608 | −37.376 | −30.625 | 1.00 | 50.68 | A | C |
| ATOM | 79 | CD | LYS | A | 13 | −29.503 | −38.928 | −30.912 | 1.00 | 59.09 | A | C |
| ATOM | 80 | CE | LYS | A | 13 | −28.149 | −39.327 | −31.606 | 1.00 | 62.01 | A | C |
| ATOM | 81 | NZ | LYS | A | 13 | −28.222 | −40.646 | −32.356 | 1.00 | 60.47 | A | N |
| ATOM | 82 | C | LYS | A | 13 | −32.599 | −35.440 | −29.035 | 1.00 | 43.37 | A | C |
| ATOM | 83 | O | LYS | A | 13 | −32.647 | −35.028 | −27.894 | 1.00 | 43.72 | A | O |
| ATOM | 84 | N | THR | A | 14 | −33.676 | −35.837 | −29.697 | 1.00 | 43.02 | A | N |
| ATOM | 85 | CA | THR | A | 14 | −35.013 | −35.820 | −29.126 | 1.00 | 42.26 | A | C |
| ATOM | 86 | CB | THR | A | 14 | −36.049 | −35.120 | −30.099 | 1.00 | 42.99 | A | C |
| ATOM | 87 | OG1 | THR | A | 14 | −35.904 | −33.701 | −29.986 | 1.00 | 40.45 | A | O |
| ATOM | 88 | CG2 | THR | A | 14 | −37.519 | −35.492 | −29.794 | 1.00 | 41.44 | A | C |
| ATOM | 89 | C | THR | A | 14 | −35.441 | −37.241 | −28.799 | 1.00 | 42.01 | A | C |
| ATOM | 90 | O | THR | A | 14 | −35.330 | −38.123 | −29.644 | 1.00 | 42.35 | A | O |
| ATOM | 91 | N | VAL | A | 15 | −35.908 | −37.478 | −27.569 | 1.00 | 40.90 | A | N |
| ATOM | 92 | CA | VAL | A | 15 | −36.346 | −38.846 | −27.192 | 1.00 | 39.44 | A | C |
| ATOM | 93 | CB | VAL | A | 15 | −35.297 | −39.522 | −26.261 | 1.00 | 39.86 | A | C |
| ATOM | 94 | CG1 | VAL | A | 15 | −34.013 | −39.848 | −27.046 | 1.00 | 34.51 | A | C |
| ATOM | 95 | CG2 | VAL | A | 15 | −34.997 | −38.621 | −24.996 | 1.00 | 37.32 | A | C |
| ATOM | 96 | C | VAL | A | 15 | −37.779 | −38.883 | −26.600 | 1.00 | 39.22 | A | C |
| ATOM | 97 | O | VAL | A | 15 | −38.288 | −37.859 | −26.124 | 1.00 | 38.62 | A | O |
| ATOM | 98 | N | SER | A | 16 | −38.443 | −40.034 | −26.653 | 1.00 | 38.75 | A | N |
| ATOM | 99 | CA | SER | A | 16 | −39.693 | −40.193 | −25.874 | 1.00 | 39.39 | A | C |
| ATOM | 100 | CB | SER | A | 16 | −40.511 | −41.380 | −26.338 | 1.00 | 39.15 | A | C |
| ATOM | 101 | OG | SER | A | 16 | −40.729 | −41.230 | −27.691 | 1.00 | 43.31 | A | O |
| ATOM | 102 | C | SER | A | 16 | −39.402 | −40.439 | −24.405 | 1.00 | 39.11 | A | C |
| ATOM | 103 | O | SER | A | 16 | −38.488 | −41.214 | −24.085 | 1.00 | 39.19 | A | O |
| ATOM | 104 | N | LEU | A | 17 | −40.218 | −39.838 | −23.532 | 1.00 | 38.27 | A | N |
| ATOM | 105 | CA | LEU | A | 17 | −40.058 | −39.952 | −22.081 | 1.00 | 37.35 | A | C |
| ATOM | 106 | CB | LEU | A | 17 | −39.700 | −38.582 | −21.507 | 1.00 | 36.35 | A | C |
| ATOM | 107 | CG | LEU | A | 17 | −38.308 | −38.048 | −21.857 | 1.00 | 33.05 | A | C |
| ATOM | 108 | CD1 | LEU | A | 17 | −38.184 | −36.605 | −21.514 | 1.00 | 25.83 | A | C |
| ATOM | 109 | CD2 | LEU | A | 17 | −37.189 | −38.879 | −21.147 | 1.00 | 31.27 | A | C |
| ATOM | 110 | C | LEU | A | 17 | −41.371 | −40.406 | −21.500 | 1.00 | 37.48 | A | C |
| ATOM | 111 | O | LEU | A | 17 | −42.378 | −39.859 | −21.864 | 1.00 | 38.44 | A | O |
| ATOM | 112 | N | ASN | A | 18 | −41.383 | −41.413 | −20.628 | 1.00 | 37.00 | A | N |
| ATOM | 113 | CA | ASN | A | 18 | −42.589 | −41.755 | −19.891 | 1.00 | 36.02 | A | C |
| ATOM | 114 | CB | ASN | A | 18 | −42.560 | −43.192 | −19.444 | 1.00 | 35.98 | A | C |
| ATOM | 115 | CG | ASN | A | 18 | −42.465 | −44.148 | −20.596 | 1.00 | 39.27 | A | C |
| ATOM | 116 | OD1 | ASN | A | 18 | −41.623 | −45.048 | −20.623 | 1.00 | 43.56 | A | O |
| ATOM | 117 | ND2 | ASN | A | 18 | −43.324 | −43.971 | −21.558 | 1.00 | 40.11 | A | N |
| ATOM | 118 | C | ASN | A | 18 | −42.687 | −40.812 | −18.693 | 1.00 | 35.67 | A | C |
| ATOM | 119 | O | ASN | A | 18 | −41.822 | −40.804 | −17.838 | 1.00 | 34.62 | A | O |
| ATOM | 120 | N | ILE | A | 19 | −43.743 | −40.004 | −18.667 | 1.00 | 35.62 | A | N |
| ATOM | 121 | CA | ILE | A | 19 | −43.935 | −38.949 | −17.666 | 1.00 | 35.75 | A | C |
| ATOM | 122 | CB | ILE | A | 19 | −43.722 | −37.554 | −18.292 | 1.00 | 35.34 | A | C |
| ATOM | 123 | CG1 | ILE | A | 19 | −44.825 | −37.201 | −19.302 | 1.00 | 33.51 | A | C |
| ATOM | 124 | CD1 | ILE | A | 19 | −44.612 | −35.848 | −19.949 | 1.00 | 34.72 | A | C |
| ATOM | 125 | CG2 | ILE | A | 19 | −42.332 | −37.528 | −18.917 | 1.00 | 33.23 | A | C |
| ATOM | 126 | C | ILE | A | 19 | −45.291 | −39.046 | −16.909 | 1.00 | 36.80 | A | C |
| ATOM | 127 | O | ILE | A | 19 | −46.081 | −39.965 | −17.145 | 1.00 | 35.92 | A | O |
| ATOM | 128 | N | SER | A | 20 | −45.498 | −38.140 | −15.963 | 1.00 | 38.36 | A | N |
| ATOM | 129 | CA | SER | A | 20 | −46.765 | −38.045 | −15.189 | 1.00 | 40.90 | A | C |
| ATOM | 130 | CB | SER | A | 20 | −46.534 | −38.472 | −13.726 | 1.00 | 40.17 | A | C |
| ATOM | 131 | OG | SER | A | 20 | −47.498 | −37.935 | −12.855 | 1.00 | 39.41 | A | O |
| ATOM | 132 | C | SER | A | 20 | −47.313 | −36.615 | −15.209 | 1.00 | 42.05 | A | C |
| ATOM | 133 | O | SER | A | 20 | −46.530 | −35.668 | −14.952 | 1.00 | 42.63 | A | O |
| ATOM | 134 | N | SER | A | 21 | −48.586 | −36.438 | −15.529 | 1.00 | 43.64 | A | N |
| ATOM | 135 | CA | SER | A | 21 | −49.219 | −35.132 | −15.353 | 1.00 | 45.31 | A | C |
| ATOM | 136 | CB | SER | A | 21 | −50.357 | −34.872 | −16.336 | 1.00 | 45.18 | A | C |
| ATOM | 137 | OG | SER | A | 21 | −51.138 | −36.000 | −16.559 | 1.00 | 45.91 | A | O |
| ATOM | 138 | C | SER | A | 21 | −49.696 | −35.055 | −13.949 | 1.00 | 45.77 | A | C |
| ATOM | 139 | O | SER | A | 21 | −50.535 | −35.811 | −13.556 | 1.00 | 45.47 | A | O |
| ATOM | 140 | N | GLU | A | 22 | −49.113 | −34.148 | −13.193 | 1.00 | 47.74 | A | N |
| ATOM | 141 | CA | GLU | A | 22 | −49.171 | −34.189 | −11.767 | 1.00 | 49.15 | A | C |
| ATOM | 142 | CB | GLU | A | 22 | −47.892 | −34.769 | −11.260 | 1.00 | 49.13 | A | C |
| ATOM | 143 | CG | GLU | A | 22 | −48.016 | −36.086 | −10.722 | 1.00 | 46.55 | A | C |
| ATOM | 144 | CD | GLU | A | 22 | −46.717 | −36.646 | −10.354 | 1.00 | 39.46 | A | C |
| ATOM | 145 | OE1 | GLU | A | 22 | −45.983 | −35.997 | −9.639 | 1.00 | 34.91 | A | O |
| ATOM | 146 | OE2 | GLU | A | 22 | −46.432 | −37.720 | −10.806 | 1.00 | 32.47 | A | O |
| ATOM | 147 | C | GLU | A | 22 | −49.162 | −32.806 | −11.279 | 1.00 | 51.23 | A | C |
| ATOM | 148 | O | GLU | A | 22 | −48.381 | −32.018 | −11.727 | 1.00 | 52.04 | A | O |
| ATOM | 149 | N | SER | A | 23 | −49.996 | −32.494 | −10.320 | 1.00 | 52.77 | A | N |
| ATOM | 150 | CA | SER | A | 23 | −50.001 | −31.133 | −9.903 | 1.00 | 54.10 | A | C |
| ATOM | 151 | CB | SER | A | 23 | −48.649 | −30.810 | −9.340 | 1.00 | 54.93 | A | C |
| ATOM | 152 | OG | SER | A | 23 | −48.179 | −31.939 | −8.645 | 1.00 | 54.76 | A | O |
| ATOM | 153 | C | SER | A | 23 | −50.282 | −30.272 | −11.111 | 1.00 | 54.42 | A | C |
| ATOM | 154 | O | SER | A | 23 | −51.075 | −30.606 | −11.970 | 1.00 | 55.29 | A | O |
| ATOM | 155 | N | GLY | A | 24 | −49.653 | −29.143 | −11.212 | 1.00 | 54.60 | A | N |
| ATOM | 156 | CA | GLY | A | 24 | −50.035 | −28.344 | −12.342 | 1.00 | 54.68 | A | C |
| ATOM | 157 | C | GLY | A | 24 | −49.544 | −28.801 | −13.683 | 1.00 | 54.45 | A | C |
| ATOM | 158 | O | GLY | A | 24 | −50.009 | −28.324 | −14.682 | 1.00 | 54.42 | A | O |
| ATOM | 159 | N | LYS | A | 25 | −48.589 | −29.717 | −13.685 | 1.00 | 53.42 | A | N |
| ATOM | 160 | CA | LYS | A | 25 | −47.626 | −29.848 | −14.742 | 1.00 | 51.96 | A | C |
| ATOM | 161 | CB | LYS | A | 25 | −46.345 | −29.213 | −14.276 | 1.00 | 51.64 | A | C |
| ATOM | 162 | CG | LYS | A | 25 | −46.385 | −28.938 | −12.870 | 1.00 | 54.15 | A | C |
| ATOM | 163 | CD | LYS | A | 25 | −45.570 | −29.847 | −12.097 | 1.00 | 57.56 | A | C |
| ATOM | 164 | CE | LYS | A | 25 | −44.768 | −29.072 | −11.100 | 1.00 | 60.94 | A | C |
| ATOM | 165 | NZ | LYS | A | 25 | −44.500 | −29.845 | −9.858 | 1.00 | 61.61 | A | N |
| ATOM | 166 | C | LYS | A | 25 | −47.270 | −31.238 | −15.143 | 1.00 | 51.03 | A | C |
| ATOM | 167 | O | LYS | A | 25 | −48.023 | −32.164 | −15.011 | 1.00 | 51.65 | A | O |
| ATOM | 168 | N | TYR | A | 26 | −46.058 | −31.349 | −15.644 | 1.00 | 48.55 | A | N |
| ATOM | 169 | CA | TYR | A | 26 | −45.481 | −32.609 | −15.998 | 1.00 | 45.54 | A | C |
| ATOM | 170 | CB | TYR | A | 26 | −45.266 | −32.653 | −17.468 | 1.00 | 45.44 | A | C |
| ATOM | 171 | CG | TYR | A | 26 | −46.525 | −32.475 | −18.221 | 1.00 | 47.36 | A | C |
| ATOM | 172 | CD1 | TYR | A | 26 | −47.208 | −33.550 | −18.707 | 1.00 | 47.06 | A | C |
| ATOM | 173 | CE1 | TYR | A | 26 | −48.330 | −33.377 | −19.398 | 1.00 | 50.44 | A | C |
| ATOM | 174 | CZ | TYR | A | 26 | −48.788 | −32.124 | −19.615 | 1.00 | 49.90 | A | C |
| ATOM | 175 | OH | TYR | A | 26 | −49.922 | −31.948 | −20.312 | 1.00 | 52.47 | A | O |
| ATOM | 176 | CE2 | TYR | A | 26 | −48.138 | −31.061 | −19.143 | 1.00 | 46.84 | A | C |
| ATOM | 177 | CD2 | TYR | A | 26 | −47.028 | −31.228 | −18.457 | 1.00 | 46.94 | A | C |
| ATOM | 178 | C | TYR | A | 26 | −44.191 | −32.834 | −15.297 | 1.00 | 43.35 | A | C |
| ATOM | 179 | O | TYR | A | 26 | −43.380 | −31.955 | −15.155 | 1.00 | 42.38 | A | O |
| ATOM | 180 | N | VAL | A | 27 | −44.026 | −34.045 | −14.834 | 1.00 | 40.47 | A | N |
| ATOM | 181 | CA | VAL | A | 27 | −42.808 | −34.391 | −14.083 | 1.00 | 38.59 | A | C |
| ATOM | 182 | CB | VAL | A | 27 | −43.103 | −34.570 | −12.556 | 1.00 | 38.99 | A | C |
| ATOM | 183 | CG1 | VAL | A | 27 | −43.789 | −33.357 | −12.001 | 1.00 | 38.60 | A | C |
| ATOM | 184 | CG2 | VAL | A | 27 | −43.963 | −35.855 | −12.313 | 1.00 | 37.66 | A | C |
| ATOM | 185 | C | VAL | A | 27 | −42.118 | −35.672 | −14.643 | 1.00 | 36.60 | A | C |
| ATOM | 186 | O | VAL | A | 27 | −42.781 | −36.552 | −15.270 | 1.00 | 34.97 | A | O |
| ATOM | 187 | N | LEU | A | 28 | −40.815 | −35.780 | −14.379 | 1.00 | 33.39 | A | N |
| ATOM | 188 | CA | LEU | A | 28 | −40.087 | −37.030 | −14.686 | 1.00 | 31.31 | A | C |
| ATOM | 189 | CB | LEU | A | 28 | −38.592 | −36.741 | −14.896 | 1.00 | 30.82 | A | C |
| ATOM | 190 | CG | LEU | A | 28 | −38.370 | −35.763 | −16.069 | 1.00 | 29.25 | A | C |
| ATOM | 191 | CD1 | LEU | A | 28 | −36.887 | −35.614 | −16.436 | 1.00 | 25.75 | A | C |
| ATOM | 192 | CD2 | LEU | A | 28 | −39.188 | −36.282 | −17.297 | 1.00 | 27.62 | A | C |
| ATOM | 193 | C | LEU | A | 28 | −40.356 | −38.195 | −13.692 | 1.00 | 29.91 | A | C |
| ATOM | 194 | O | LEU | A | 28 | −39.512 | −38.572 | −12.864 | 1.00 | 28.72 | A | O |
| ATOM | 195 | N | ARG | A | 29 | −41.548 | −38.759 | −13.821 | 1.00 | 29.17 | A | N |
| ATOM | 196 | CA | ARG | A | 29 | −41.983 | −39.934 | −13.093 | 1.00 | 29.07 | A | C |
| ATOM | 197 | CB | ARG | A | 29 | −43.031 | −39.566 | −12.002 | 1.00 | 28.30 | A | C |
| ATOM | 198 | CG | ARG | A | 29 | −43.815 | −40.779 | −11.438 | 1.00 | 29.82 | A | C |
| ATOM | 199 | CD | ARG | A | 29 | −44.958 | −40.532 | −10.414 | 1.00 | 29.04 | A | C |
| ATOM | 200 | NE | ARG | A | 29 | −44.832 | −39.314 | −9.598 | 1.00 | 28.43 | A | N |
| ATOM | 201 | CZ | ARG | A | 29 | −44.170 | −39.129 | −8.449 | 1.00 | 27.95 | A | C |
| ATOM | 202 | NH1 | ARG | A | 29 | −43.416 | −40.064 | −7.875 | 1.00 | 28.57 | A | N |
| ATOM | 203 | NH2 | ARG | A | 29 | −44.232 | −37.925 | −7.885 | 1.00 | 29.61 | A | N |
| ATOM | 204 | C | ARG | A | 29 | −42.556 | −40.961 | −14.088 | 1.00 | 29.23 | A | C |
| ATOM | 205 | O | ARG | A | 29 | −43.510 | −40.685 | −14.745 | 1.00 | 28.41 | A | O |
| ATOM | 206 | N | ASP | A | 30 | −41.990 | −42.170 | −14.153 | 1.00 | 30.20 | A | N |
| ATOM | 207 | CA | ASP | A | 30 | −42.364 | −43.158 | −15.151 | 1.00 | 30.03 | A | C |
| ATOM | 208 | CB | ASP | A | 30 | −41.100 | −43.849 | −15.701 | 1.00 | 29.34 | A | C |
| ATOM | 209 | CG | ASP | A | 30 | −41.383 | −44.840 | −16.878 | 1.00 | 30.84 | A | C |
| ATOM | 210 | OD1 | ASP | A | 30 | −42.525 | −45.362 | −17.072 | 1.00 | 29.94 | A | O |
| ATOM | 211 | OD2 | ASP | A | 30 | −40.425 | −45.107 | −17.621 | 1.00 | 27.79 | A | O |
| ATOM | 212 | C | ASP | A | 30 | −43.360 | −44.123 | −14.524 | 1.00 | 30.55 | A | C |
| ATOM | 213 | O | ASP | A | 30 | −43.089 | −44.784 | −13.525 | 1.00 | 29.55 | A | O |
| ATOM | 214 | N | LEU | A | 31 | −44.531 | −44.182 | −15.129 | 1.00 | 31.84 | A | N |
| ATOM | 215 | CA | LEU | A | 31 | −45.673 | −44.804 | −14.522 | 1.00 | 33.66 | A | C |
| ATOM | 216 | CB | LEU | A | 31 | −46.741 | −43.758 | −14.269 | 1.00 | 33.92 | A | C |
| ATOM | 217 | CG | LEU | A | 31 | −47.021 | −43.233 | −12.839 | 1.00 | 37.06 | A | C |
| ATOM | 218 | CD1 | LEU | A | 31 | −45.876 | −43.230 | −11.897 | 1.00 | 35.24 | A | C |
| ATOM | 219 | CD2 | LEU | A | 31 | −47.674 | −41.810 | −12.854 | 1.00 | 38.45 | A | C |
| ATOM | 220 | C | LEU | A | 31 | −46.189 | −45.830 | −15.479 | 1.00 | 35.24 | A | C |
| ATOM | 221 | O | LEU | A | 31 | −47.292 | −46.339 | −15.306 | 1.00 | 36.18 | A | O |
| ATOM | 222 | N | SER | A | 32 | −45.347 | −46.150 | −16.474 | 1.00 | 36.86 | A | N |
| ATOM | 223 | CA | SER | A | 32 | −45.652 | −47.055 | −17.588 | 1.00 | 36.85 | A | C |
| ATOM | 224 | CB | SER | A | 32 | −45.024 | −46.556 | −18.925 | 1.00 | 36.60 | A | C |
| ATOM | 225 | OG | SER | A | 32 | −43.606 | −46.774 | −18.972 | 1.00 | 38.59 | A | O |
| ATOM | 226 | C | SER | A | 32 | −45.197 | −48.468 | −17.297 | 1.00 | 37.03 | A | C |
| ATOM | 227 | O | SER | A | 32 | −45.544 | −49.365 | −18.061 | 1.00 | 36.94 | A | O |
| ATOM | 228 | N | LYS | A | 33 | −44.434 | −48.691 | −16.225 | 1.00 | 37.38 | A | N |
| ATOM | 229 | CA | LYS | A | 33 | −43.866 | −50.038 | −16.026 | 1.00 | 38.79 | A | C |
| ATOM | 230 | CB | LYS | A | 33 | −42.573 | −50.035 | −15.186 | 1.00 | 38.26 | A | C |
| ATOM | 231 | CG | LYS | A | 33 | −41.473 | −49.071 | −15.689 | 1.00 | 34.67 | A | C |
| ATOM | 232 | CD | LYS | A | 33 | −41.118 | −49.392 | −17.134 | 1.00 | 30.21 | A | C |
| ATOM | 233 | CE | LYS | A | 33 | −39.984 | −48.532 | −17.680 | 1.00 | 29.07 | A | C |
| ATOM | 234 | NZ | LYS | A | 33 | −39.732 | −48.697 | −19.185 | 1.00 | 23.25 | A | N |
| ATOM | 235 | C | LYS | A | 33 | −44.895 | −51.052 | −15.503 | 1.00 | 41.30 | A | C |
| ATOM | 236 | O | LYS | A | 33 | −45.622 | −50.798 | −14.530 | 1.00 | 40.23 | A | O |
| ATOM | 237 | N | PRO | A | 34 | −44.958 | −52.230 | −16.147 | 1.00 | 44.22 | A | N |
| ATOM | 238 | CA | PRO | A | 34 | −46.201 | −52.950 | −15.790 | 1.00 | 45.38 | A | C |
| ATOM | 239 | CB | PRO | A | 34 | −46.352 | −54.000 | −16.926 | 1.00 | 47.30 | A | C |
| ATOM | 240 | CG | PRO | A | 34 | −44.809 | −54.186 | −17.497 | 1.00 | 48.14 | A | C |
| ATOM | 241 | CD | PRO | A | 34 | −43.951 | −53.092 | −16.834 | 1.00 | 44.53 | A | C |
| ATOM | 242 | C | PRO | A | 34 | −46.125 | −53.585 | −14.377 | 1.00 | 44.92 | A | C |
| ATOM | 243 | O | PRO | A | 34 | −47.134 | −54.070 | −13.845 | 1.00 | 45.69 | A | O |
| ATOM | 244 | N | THR | A | 35 | −44.963 | −53.546 | −13.741 | 1.00 | 42.51 | A | N |
| ATOM | 245 | CA | THR | A | 35 | −44.926 | −54.019 | −12.364 | 1.00 | 40.40 | A | C |
| ATOM | 246 | CB | THR | A | 35 | −43.499 | −54.464 | −12.004 | 1.00 | 41.17 | A | C |
| ATOM | 247 | OG1 | THR | A | 35 | −42.610 | −53.388 | −12.332 | 1.00 | 40.39 | A | O |
| ATOM | 248 | CG2 | THR | A | 35 | −43.107 | −55.773 | −12.741 | 1.00 | 39.31 | A | C |
| ATOM | 249 | C | THR | A | 35 | −45.346 | −52.956 | −11.315 | 1.00 | 38.28 | A | C |
| ATOM | 250 | O | THR | A | 35 | −45.433 | −53.269 | −10.120 | 1.00 | 36.30 | A | O |
| ATOM | 251 | N | GLY | A | 36 | −45.536 | −51.702 | −11.755 | 1.00 | 36.35 | A | N |
| ATOM | 252 | CA | GLY | A | 36 | −45.713 | −50.583 | −10.830 | 1.00 | 32.77 | A | C |
| ATOM | 253 | C | GLY | A | 36 | −44.426 | −49.842 | −10.507 | 1.00 | 31.89 | A | C |
| ATOM | 254 | O | GLY | A | 36 | −44.456 | −48.780 | −9.912 | 1.00 | 31.63 | A | O |
| ATOM | 255 | N | THR | A | 37 | −43.266 | −50.351 | −10.924 | 1.00 | 30.83 | A | N |
| ATOM | 256 | CA | THR | A | 37 | −42.003 | −49.747 | −10.482 | 1.00 | 27.70 | A | C |
| ATOM | 257 | CB | THR | A | 37 | −40.819 | −50.642 | −10.820 | 1.00 | 27.55 | A | C |
| ATOM | 258 | OG1 | THR | A | 37 | −40.960 | −51.876 | −10.095 | 1.00 | 28.01 | A | O |
| ATOM | 259 | CG2 | THR | A | 37 | −39.494 | −49.979 | −10.501 | 1.00 | 23.29 | A | C |
| ATOM | 260 | C | THR | A | 37 | −41.851 | −48.380 | −11.106 | 1.00 | 26.95 | A | C |
| ATOM | 261 | O | THR | A | 37 | −41.941 | −48.260 | −12.281 | 1.00 | 26.14 | A | O |
| ATOM | 262 | N | GLN | A | 38 | −41.525 | −47.385 | −10.329 | 1.00 | 26.09 | A | N |
| ATOM | 263 | CA | GLN | A | 38 | −41.319 | −46.059 | −10.826 | 1.00 | 26.76 | A | C |
| ATOM | 264 | CB | GLN | A | 38 | −41.863 | −45.066 | −9.816 | 1.00 | 27.60 | A | C |
| ATOM | 265 | CG | GLN | A | 38 | −43.300 | −44.836 | −9.957 | 1.00 | 30.56 | A | C |
| ATOM | 266 | CD | GLN | A | 38 | −43.913 | −44.030 | −8.864 | 1.00 | 34.02 | A | C |
| ATOM | 267 | OE1 | GLN | A | 38 | −43.367 | −43.074 | −8.393 | 1.00 | 35.21 | A | O |
| ATOM | 268 | NE2 | GLN | A | 38 | −45.077 | −44.404 | −8.485 | 1.00 | 31.60 | A | N |
| ATOM | 269 | C | GLN | A | 38 | −39.881 | −45.752 | −11.122 | 1.00 | 25.09 | A | C |
| ATOM | 270 | O | GLN | A | 38 | −39.025 | −46.182 | −10.432 | 1.00 | 26.13 | A | O |
| ATOM | 271 | N | ILE | A | 39 | −39.629 | −45.004 | −12.176 | 1.00 | 23.80 | A | N |
| ATOM | 272 | CA | ILE | A | 39 | −38.346 | −44.336 | −12.348 | 1.00 | 24.26 | A | C |
| ATOM | 273 | CB | ILE | A | 39 | −37.699 | −44.596 | −13.772 | 1.00 | 24.68 | A | C |
| ATOM | 274 | CG1 | ILE | A | 39 | −37.395 | −46.080 | −14.014 | 1.00 | 21.84 | A | C |
| ATOM | 275 | CD1 | ILE | A | 39 | −38.507 | −46.858 | −14.474 | 1.00 | 21.10 | A | C |
| ATOM | 276 | CG2 | ILE | A | 39 | −36.388 | −43.745 | −13.945 | 1.00 | 24.04 | A | C |
| ATOM | 277 | C | ILE | A | 39 | −38.587 | −42.826 | −12.122 | 1.00 | 24.60 | A | C |
| ATOM | 278 | O | ILE | A | 39 | −39.521 | −42.255 | −12.681 | 1.00 | 25.49 | A | O |
| ATOM | 279 | N | ILE | A | 40 | −37.804 | −42.171 | −11.281 | 1.00 | 24.30 | A | N |
| ATOM | 280 | CA | ILE | A | 40 | −38.138 | −40.820 | −10.904 | 1.00 | 24.67 | A | C |
| ATOM | 281 | CB | ILE | A | 40 | −38.760 | −40.776 | −9.481 | 1.00 | 25.40 | A | C |
| ATOM | 282 | CG1 | ILE | A | 40 | −39.778 | −41.902 | −9.248 | 1.00 | 26.78 | A | C |
| ATOM | 283 | CD1 | ILE | A | 40 | −40.256 | −41.969 | −7.776 | 1.00 | 28.72 | A | C |
| ATOM | 284 | CG2 | ILE | A | 40 | −39.456 | −39.440 | −9.198 | 1.00 | 25.40 | A | C |
| ATOM | 285 | C | ILE | A | 40 | −36.831 | −40.033 | −10.917 | 1.00 | 25.31 | A | C |
| ATOM | 286 | O | ILE | A | 40 | −35.881 | −40.469 | −10.303 | 1.00 | 25.86 | A | O |
| ATOM | 287 | N | THR | A | 41 | −36.785 | −38.864 | −11.577 | 1.00 | 25.27 | A | N |
| ATOM | 288 | CA | THR | A | 41 | −35.535 | −38.168 | −11.811 | 1.00 | 24.40 | A | C |
| ATOM | 289 | CB | THR | A | 41 | −35.271 | −38.016 | −13.322 | 1.00 | 23.99 | A | C |
| ATOM | 290 | OG1 | THR | A | 41 | −35.523 | −39.264 | −13.986 | 1.00 | 24.89 | A | O |
| ATOM | 291 | CG2 | THR | A | 41 | −33.870 | −37.563 | −13.592 | 1.00 | 20.66 | A | C |
| ATOM | 292 | C | THR | A | 41 | −35.545 | −36.793 | −11.150 | 1.00 | 26.35 | A | C |
| ATOM | 293 | O | THR | A | 41 | −36.499 | −36.004 | −11.319 | 1.00 | 26.67 | A | O |
| ATOM | 294 | N | TYR | A | 42 | −34.488 | −36.490 | −10.401 | 1.00 | 26.81 | A | N |
| ATOM | 295 | CA | TYR | A | 42 | −34.470 | −35.288 | −9.624 | 1.00 | 28.66 | A | C |
| ATOM | 296 | CB | TYR | A | 42 | −34.363 | −35.615 | −8.121 | 1.00 | 29.19 | A | C |
| ATOM | 297 | CG | TYR | A | 42 | −35.602 | −36.234 | −7.554 | 1.00 | 28.72 | A | C |
| ATOM | 298 | CD1 | TYR | A | 42 | −35.834 | −37.620 | −7.674 | 1.00 | 29.76 | A | C |
| ATOM | 299 | CE1 | TYR | A | 42 | −36.985 | −38.198 | −7.145 | 1.00 | 30.70 | A | C |
| ATOM | 300 | CZ | TYR | A | 42 | −37.913 | −37.389 | −6.473 | 1.00 | 32.18 | A | C |
| ATOM | 301 | OH | TYR | A | 42 | −39.044 | −37.951 | −5.962 | 1.00 | 34.06 | A | O |
| ATOM | 302 | CE2 | TYR | A | 42 | −37.732 | −36.002 | −6.366 | 1.00 | 29.34 | A | C |
| ATOM | 303 | CD2 | TYR | A | 42 | −36.556 | −35.441 | −6.883 | 1.00 | 28.41 | A | C |
| ATOM | 304 | C | TYR | A | 42 | −33.275 | −34.456 | −10.028 | 1.00 | 30.08 | A | C |
| ATOM | 305 | O | TYR | A | 42 | −32.293 | −34.980 | −10.550 | 1.00 | 29.14 | A | O |
| ATOM | 306 | N | ASP | A | 43 | −33.356 | −33.160 | −9.738 | 1.00 | 31.69 | A | N |
| ATOM | 307 | CA | ASP | A | 43 | −32.296 | −32.216 | −10.075 | 1.00 | 33.46 | A | C |
| ATOM | 308 | CB | ASP | A | 43 | −32.867 | −31.090 | −10.989 | 1.00 | 32.64 | A | C |
| ATOM | 309 | CG | ASP | A | 43 | −31.851 | −29.958 | −11.296 | 1.00 | 32.89 | A | C |
| ATOM | 310 | OD1 | ASP | A | 43 | −30.696 | −29.966 | −10.757 | 1.00 | 30.15 | A | O |
| ATOM | 311 | OD2 | ASP | A | 43 | −32.236 | −29.054 | −12.087 | 1.00 | 32.73 | A | O |
| ATOM | 312 | C | ASP | A | 43 | −31.740 | −31.685 | −8.754 | 1.00 | 34.58 | A | C |
| ATOM | 313 | O | ASP | A | 43 | −32.470 | −31.124 | −7.943 | 1.00 | 35.27 | A | O |
| ATOM | 314 | N | LEU | A | 44 | −30.453 | −31.866 | −8.521 | 1.00 | 36.27 | A | N |
| ATOM | 315 | CA | LEU | A | 44 | −29.884 | −31.438 | −7.245 | 1.00 | 37.65 | A | C |
| ATOM | 316 | CB | LEU | A | 44 | −28.720 | −32.371 | −6.881 | 1.00 | 37.26 | A | C |
| ATOM | 317 | CG | LEU | A | 44 | −28.521 | −33.054 | −5.509 | 1.00 | 39.07 | A | C |
| ATOM | 318 | CD1 | LEU | A | 44 | −29.742 | −33.120 | −4.619 | 1.00 | 36.83 | A | C |
| ATOM | 319 | CD2 | LEU | A | 44 | −27.831 | −34.449 | −5.635 | 1.00 | 37.50 | A | C |
| ATOM | 320 | C | LEU | A | 44 | −29.472 | −29.939 | −7.294 | 1.00 | 38.87 | A | C |
| ATOM | 321 | O | LEU | A | 44 | −29.162 | −29.343 | −6.279 | 1.00 | 39.39 | A | O |
| ATOM | 322 | N | GLN | A | 45 | −29.471 | −29.328 | −8.482 | 1.00 | 40.24 | A | N |
| ATOM | 323 | CA | GLN | A | 45 | −28.976 | −27.955 | −8.653 | 1.00 | 40.83 | A | C |
| ATOM | 324 | CB | GLN | A | 45 | −30.003 | −26.930 | −8.127 | 1.00 | 40.93 | A | C |
| ATOM | 325 | CG | GLN | A | 45 | −31.400 | −27.042 | −8.843 | 1.00 | 43.55 | A | C |
| ATOM | 326 | CD | GLN | A | 45 | −32.557 | −26.463 | −8.026 | 1.00 | 46.24 | A | C |
| ATOM | 327 | OE1 | GLN | A | 45 | −32.440 | −26.239 | −6.825 | 1.00 | 48.19 | A | O |
| ATOM | 328 | NE2 | GLN | A | 45 | −33.669 | −26.215 | −8.680 | 1.00 | 44.67 | A | N |
| ATOM | 329 | C | GLN | A | 45 | −27.607 | −27.785 | −8.002 | 1.00 | 41.18 | A | C |
| ATOM | 330 | O | GLN | A | 45 | −27.368 | −26.809 | −7.304 | 1.00 | 42.73 | A | O |
| ATOM | 331 | N | ASN | A | 46 | −26.710 | −28.738 | −8.202 | 1.00 | 40.77 | A | N |
| ATOM | 332 | CA | ASN | A | 46 | −25.305 | −28.565 | −7.818 | 1.00 | 41.87 | A | C |
| ATOM | 333 | CB | ASN | A | 46 | −24.676 | −27.325 | −8.485 | 1.00 | 41.62 | A | C |
| ATOM | 334 | CG | ASN | A | 46 | −24.762 | −27.374 | −10.029 | 1.00 | 42.52 | A | C |
| ATOM | 335 | OD1 | ASN | A | 46 | −25.384 | −26.511 | −10.647 | 1.00 | 42.00 | A | O |
| ATOM | 336 | ND2 | ASN | A | 46 | −24.173 | −28.426 | −10.642 | 1.00 | 39.75 | A | N |
| ATOM | 337 | C | ASN | A | 46 | −24.977 | −28.607 | −6.340 | 1.00 | 43.16 | A | C |
| ATOM | 338 | O | ASN | A | 46 | −23.805 | −28.419 | −6.002 | 1.00 | 42.88 | A | O |
| ATOM | 339 | N | ARG | A | 47 | −25.992 | −28.859 | −5.485 | 1.00 | 44.54 | A | N |
| ATOM | 340 | CA | ARG | A | 47 | −25.793 | −29.192 | −4.062 | 1.00 | 46.84 | A | C |
| ATOM | 341 | CB | ARG | A | 47 | −27.013 | −28.809 | −3.177 | 1.00 | 47.30 | A | C |
| ATOM | 342 | CG | ARG | A | 47 | −28.061 | −27.854 | −3.818 | 1.00 | 52.65 | A | C |
| ATOM | 343 | CD | ARG | A | 47 | −28.528 | −26.620 | −2.951 | 1.00 | 60.12 | A | C |
| ATOM | 344 | NE | ARG | A | 47 | −27.637 | −25.455 | −3.151 | 1.00 | 65.09 | A | N |
| ATOM | 345 | CZ | ARG | A | 47 | −27.824 | −24.450 | −4.026 | 1.00 | 67.61 | A | C |
| ATOM | 346 | NH1 | ARG | A | 47 | −28.902 | −24.400 | −4.820 | 1.00 | 67.70 | A | N |
| ATOM | 347 | NH2 | ARG | A | 47 | −26.908 | −23.483 | −4.120 | 1.00 | 67.50 | A | N |
| ATOM | 348 | C | ARG | A | 47 | −25.431 | −30.704 | −3.904 | 1.00 | 47.66 | A | C |
| ATOM | 349 | O | ARG | A | 47 | −25.563 | −31.500 | −4.854 | 1.00 | 47.75 | A | O |
| ATOM | 350 | N | GLU | A | 48 | −24.958 | −31.100 | −2.723 | 1.00 | 48.47 | A | N |
| ATOM | 351 | CA | GLU | A | 48 | −24.549 | −32.484 | −2.482 | 1.00 | 49.38 | A | C |
| ATOM | 352 | CB | GLU | A | 48 | −23.016 | −32.618 | −2.288 | 1.00 | 49.89 | A | C |
| ATOM | 353 | CG | GLU | A | 48 | −22.140 | −31.848 | −3.281 | 1.00 | 53.97 | A | C |
| ATOM | 354 | CD | GLU | A | 48 | −20.699 | −32.390 | −3.369 | 1.00 | 58.12 | A | C |
| ATOM | 355 | OE1 | GLU | A | 48 | −20.138 | −32.504 | −4.487 | 1.00 | 58.48 | A | O |
| ATOM | 356 | OE2 | GLU | A | 48 | −20.118 | −32.708 | −2.314 | 1.00 | 60.70 | A | O |
| ATOM | 357 | C | GLU | A | 48 | −25.241 | −33.080 | −1.258 | 1.00 | 49.02 | A | C |
| ATOM | 358 | O | GLU | A | 48 | −24.821 | −34.136 | −0.773 | 1.00 | 48.86 | A | O |
| ATOM | 359 | N | TYR | A | 49 | −26.282 | −32.411 | −0.752 | 1.00 | 48.71 | A | N |
| ATOM | 360 | CA | TYR | A | 49 | −26.949 | −32.832 | 0.497 | 1.00 | 48.43 | A | C |
| ATOM | 361 | CB | TYR | A | 49 | −26.434 | −32.015 | 1.710 | 1.00 | 49.21 | A | C |
| ATOM | 362 | CG | TYR | A | 49 | −26.684 | −30.530 | 1.510 | 1.00 | 53.66 | A | C |
| ATOM | 363 | CD1 | TYR | A | 49 | −25.753 | −29.740 | 0.794 | 1.00 | 57.40 | A | C |
| ATOM | 364 | CE1 | TYR | A | 49 | −25.994 | −28.371 | 0.564 | 1.00 | 61.43 | A | C |
| ATOM | 365 | CZ | TYR | A | 49 | −27.197 | −27.783 | 1.046 | 1.00 | 63.54 | A | C |
| ATOM | 366 | OH | TYR | A | 49 | −27.421 | −26.423 | 0.803 | 1.00 | 65.65 | A | O |
| ATOM | 367 | CE2 | TYR | A | 49 | −28.150 | −28.560 | 1.742 | 1.00 | 59.44 | A | C |
| ATOM | 368 | CD2 | TYR | A | 49 | −27.883 | −29.918 | 1.975 | 1.00 | 56.66 | A | C |
| ATOM | 369 | C | TYR | A | 49 | −28.438 | −32.632 | 0.350 | 1.00 | 47.07 | A | C |
| ATOM | 370 | O | TYR | A | 49 | −28.896 | −31.946 | −0.594 | 1.00 | 46.06 | A | O |
| ATOM | 371 | N | ASN | A | 50 | −29.192 | −33.194 | 1.315 | 1.00 | 46.10 | A | N |
| ATOM | 372 | CA | ASN | A | 50 | −30.657 | −33.095 | 1.331 | 1.00 | 43.95 | A | C |
| ATOM | 373 | CB | ASN | A | 50 | −31.101 | −31.597 | 1.518 | 1.00 | 44.54 | A | C |
| ATOM | 374 | CG | ASN | A | 50 | −32.582 | −31.439 | 1.975 | 1.00 | 46.46 | A | C |
| ATOM | 375 | OD1 | ASN | A | 50 | −33.116 | −32.261 | 2.725 | 1.00 | 47.44 | A | O |
| ATOM | 376 | ND2 | ASN | A | 50 | −33.238 | −30.373 | 1.511 | 1.00 | 48.73 | A | N |
| ATOM | 377 | C | ASN | A | 50 | −31.196 | −33.744 | 0.023 | 1.00 | 41.68 | A | C |
| ATOM | 378 | O | ASN | A | 50 | −32.011 | −33.161 | −0.691 | 1.00 | 40.51 | A | O |
| ATOM | 379 | N | LEU | A | 51 | −30.711 | −34.952 | −0.266 | 1.00 | 39.34 | A | N |
| ATOM | 380 | CA | LEU | A | 51 | −31.135 | −35.742 | −1.434 | 1.00 | 38.05 | A | C |
| ATOM | 381 | CB | LEU | A | 51 | −30.272 | −37.011 | −1.608 | 1.00 | 36.68 | A | C |
| ATOM | 382 | CG | LEU | A | 51 | −28.736 | −36.853 | −1.766 | 1.00 | 38.02 | A | C |
| ATOM | 383 | CD1 | LEU | A | 51 | −28.040 | −37.730 | −2.821 | 1.00 | 30.41 | A | C |
| ATOM | 384 | CD2 | LEU | A | 51 | −28.301 | −35.401 | −1.953 | 1.00 | 38.51 | A | C |
| ATOM | 385 | C | LEU | A | 51 | −32.609 | −36.125 | −1.302 | 1.00 | 37.04 | A | C |
| ATOM | 386 | O | LEU | A | 51 | −33.115 | −36.208 | −0.183 | 1.00 | 38.60 | A | O |
| ATOM | 387 | N | PRO | A | 52 | −33.296 | −36.391 | −2.426 | 1.00 | 35.16 | A | N |
| ATOM | 388 | CA | PRO | A | 52 | −32.736 | −36.442 | −3.785 | 1.00 | 35.00 | A | C |
| ATOM | 389 | CB | PRO | A | 52 | −33.631 | −37.467 | −4.472 | 1.00 | 34.76 | A | C |
| ATOM | 390 | CG | PRO | A | 52 | −34.968 | −37.210 | −3.835 | 1.00 | 35.21 | A | C |
| ATOM | 391 | CD | PRO | A | 52 | −34.680 | −36.861 | −2.384 | 1.00 | 33.35 | A | C |
| ATOM | 392 | C | PRO | A | 52 | −32.741 | −35.098 | −4.564 | 1.00 | 34.93 | A | C |
| ATOM | 393 | O | PRO | A | 52 | −32.065 | −34.989 | −5.602 | 1.00 | 35.60 | A | O |
| ATOM | 394 | N | GLY | A | 53 | −33.486 | −34.105 | −4.068 | 1.00 | 34.24 | A | N |
| ATOM | 395 | CA | GLY | A | 53 | −33.572 | −32.778 | −4.661 | 1.00 | 33.02 | A | C |
| ATOM | 396 | C | GLY | A | 53 | −34.949 | −32.535 | −5.260 | 1.00 | 32.97 | A | C |
| ATOM | 397 | O | GLY | A | 53 | −35.952 | −33.093 | −4.807 | 1.00 | 33.28 | A | O |
| ATOM | 398 | N | THR | A | 54 | −34.967 | −31.739 | −6.320 | 1.00 | 32.27 | A | N |
| ATOM | 399 | CA | THR | A | 54 | −36.160 | −31.260 | −6.945 | 1.00 | 32.37 | A | C |
| ATOM | 400 | CB | THR | A | 54 | −35.947 | −29.811 | −7.437 | 1.00 | 32.98 | A | C |
| ATOM | 401 | OG1 | THR | A | 54 | −35.393 | −29.041 | −6.360 | 1.00 | 34.33 | A | O |
| ATOM | 402 | CG2 | THR | A | 54 | −37.276 | −29.167 | −8.007 | 1.00 | 31.11 | A | C |
| ATOM | 403 | C | THR | A | 54 | −36.605 | −32.137 | −8.106 | 1.00 | 32.94 | A | C |
| ATOM | 404 | O | THR | A | 54 | −35.878 | −32.380 | −9.087 | 1.00 | 31.68 | A | O |
| ATOM | 405 | N | LEU | A | 55 | −37.838 | −32.586 | −7.987 | 1.00 | 33.67 | A | N |
| ATOM | 406 | CA | LEU | A | 55 | −38.430 | −33.378 | −8.995 | 1.00 | 35.36 | A | C |
| ATOM | 407 | CB | LEU | A | 55 | −39.855 | −33.704 | −8.577 | 1.00 | 34.52 | A | C |
| ATOM | 408 | CG | LEU | A | 55 | −40.594 | −34.680 | −9.486 | 1.00 | 37.35 | A | C |
| ATOM | 409 | CD1 | LEU | A | 55 | −39.853 | −36.006 | −9.710 | 1.00 | 32.36 | A | C |
| ATOM | 410 | CD2 | LEU | A | 55 | −41.990 | −34.945 | −8.959 | 1.00 | 38.13 | A | C |
| ATOM | 411 | C | LEU | A | 55 | −38.335 | −32.533 | −10.275 | 1.00 | 36.98 | A | C |
| ATOM | 412 | O | LEU | A | 55 | −38.680 | −31.346 | −10.269 | 1.00 | 37.92 | A | O |
| ATOM | 413 | N | VAL | A | 56 | −37.811 | −33.123 | −11.355 | 1.00 | 37.30 | A | N |
| ATOM | 414 | CA | VAL | A | 56 | −37.657 | −32.406 | −12.591 | 1.00 | 36.64 | A | C |
| ATOM | 415 | CB | VAL | A | 56 | −36.625 | −33.113 | −13.515 | 1.00 | 36.80 | A | C |
| ATOM | 416 | CG1 | VAL | A | 56 | −36.762 | −32.623 | −14.916 | 1.00 | 34.94 | A | C |
| ATOM | 417 | CG2 | VAL | A | 56 | −35.191 | −32.844 | −13.013 | 1.00 | 32.55 | A | C |
| ATOM | 418 | C | VAL | A | 56 | −39.034 | −32.199 | −13.231 | 1.00 | 38.20 | A | C |
| ATOM | 419 | O | VAL | A | 56 | −39.807 | −33.136 | −13.400 | 1.00 | 38.95 | A | O |
| ATOM | 420 | N | SER | A | 57 | −39.370 | −30.966 | −13.578 | 1.00 | 40.18 | A | N |
| ATOM | 421 | CA | SER | A | 57 | −40.712 | −30.721 | −14.141 | 1.00 | 42.41 | A | C |
| ATOM | 422 | CB | SER | A | 57 | −41.729 | −30.414 | −13.036 | 1.00 | 41.73 | A | C |
| ATOM | 423 | OG | SER | A | 57 | −41.165 | −29.456 | −12.199 | 1.00 | 42.33 | A | O |
| ATOM | 424 | C | SER | A | 57 | −40.771 | −29.642 | −15.221 | 1.00 | 42.87 | A | C |
| ATOM | 425 | O | SER | A | 57 | −39.889 | −28.786 | −15.312 | 1.00 | 42.53 | A | O |
| ATOM | 426 | N | SER | A | 58 | −41.805 | −29.725 | −16.046 | 1.00 | 43.46 | A | N |
| ATOM | 427 | CA | SER | A | 58 | −42.064 | −28.689 | −17.021 | 1.00 | 45.24 | A | C |
| ATOM | 428 | CB | SER | A | 58 | −41.435 | −29.040 | −18.379 | 1.00 | 44.87 | A | C |
| ATOM | 429 | OG | SER | A | 58 | −42.368 | −28.988 | −19.446 | 1.00 | 43.17 | A | O |
| ATOM | 430 | C | SER | A | 58 | −43.572 | −28.385 | −17.143 | 1.00 | 46.75 | A | C |
| ATOM | 431 | O | SER | A | 58 | −44.444 | −29.258 | −16.870 | 1.00 | 45.93 | A | O |
| ATOM | 432 | N | THR | A | 59 | −43.872 | −27.155 | −17.572 | 1.00 | 47.81 | A | N |
| ATOM | 433 | CA | THR | A | 59 | −45.272 | −26.786 | −17.788 | 1.00 | 48.79 | A | C |
| ATOM | 434 | CB | THR | A | 59 | −45.480 | −25.278 | −17.829 | 1.00 | 49.03 | A | C |
| ATOM | 435 | OG1 | THR | A | 59 | −44.902 | −24.807 | −19.041 | 1.00 | 49.03 | A | O |
| ATOM | 436 | CG2 | THR | A | 59 | −44.827 | −24.562 | −16.589 | 1.00 | 48.04 | A | C |
| ATOM | 437 | C | THR | A | 59 | −45.820 | −27.435 | −19.055 | 1.00 | 48.93 | A | C |
| ATOM | 438 | O | THR | A | 59 | −47.025 | −27.449 | −19.250 | 1.00 | 49.98 | A | O |
| ATOM | 439 | N | THR | A | 60 | −44.956 | −28.002 | −19.889 | 1.00 | 48.95 | A | N |
| ATOM | 440 | CA | THR | A | 60 | −45.416 | −28.777 | −21.048 | 1.00 | 49.54 | A | C |
| ATOM | 441 | CB | THR | A | 60 | −44.976 | −28.152 | −22.416 | 1.00 | 50.77 | A | C |
| ATOM | 442 | OG1 | THR | A | 60 | −43.537 | −27.921 | −22.449 | 1.00 | 50.15 | A | O |
| ATOM | 443 | CG2 | THR | A | 60 | −45.795 | −26.834 | −22.706 | 1.00 | 49.54 | A | C |
| ATOM | 444 | C | THR | A | 60 | −44.927 | −30.207 | −21.074 | 1.00 | 49.42 | A | C |
| ATOM | 445 | O | THR | A | 60 | −44.079 | −30.621 | −20.300 | 1.00 | 49.57 | A | O |
| ATOM | 446 | N | ASN | A | 61 | −45.521 | −30.947 | −21.987 | 1.00 | 48.77 | A | N |
| ATOM | 447 | CA | ASN | A | 61 | −45.036 | −32.187 | −22.505 | 1.00 | 47.80 | A | C |
| ATOM | 448 | CB | ASN | A | 61 | −45.682 | −32.345 | −23.885 | 1.00 | 48.54 | A | C |
| ATOM | 449 | CG | ASN | A | 61 | −46.190 | −33.713 | −24.078 | 1.00 | 52.04 | A | C |
| ATOM | 450 | OD1 | ASN | A | 61 | −46.532 | −34.150 | −25.180 | 1.00 | 52.14 | A | O |
| ATOM | 451 | ND2 | ASN | A | 61 | −46.237 | −34.448 | −22.960 | 1.00 | 58.59 | A | N |
| ATOM | 452 | C | ASN | A | 61 | −43.527 | −32.339 | −22.754 | 1.00 | 46.83 | A | C |
| ATOM | 453 | O | ASN | A | 61 | −43.056 | −33.455 | −23.094 | 1.00 | 45.82 | A | O |
| ATOM | 454 | N | GLN | A | 62 | −42.780 | −31.234 | −22.648 | 1.00 | 45.02 | A | N |
| ATOM | 455 | CA | GLN | A | 62 | −41.465 | −31.161 | −23.273 | 1.00 | 43.93 | A | C |
| ATOM | 456 | CB | GLN | A | 62 | −41.479 | −30.234 | −24.529 | 1.00 | 44.43 | A | C |
| ATOM | 457 | CG | GLN | A | 62 | −42.303 | −30.766 | −25.762 | 1.00 | 49.48 | A | C |
| ATOM | 458 | CD | GLN | A | 62 | −41.858 | −30.185 | −27.192 | 1.00 | 56.15 | A | C |
| ATOM | 459 | OE1 | GLN | A | 62 | −40.951 | −29.336 | −27.299 | 1.00 | 55.55 | A | O |
| ATOM | 460 | NE2 | GLN | A | 62 | −42.511 | −30.683 | −28.274 | 1.00 | 54.68 | A | N |
| ATOM | 461 | C | GLN | A | 62 | −40.379 | −30.760 | −22.285 | 1.00 | 42.39 | A | C |
| ATOM | 462 | O | GLN | A | 62 | −40.507 | −29.738 | −21.575 | 1.00 | 41.48 | A | O |
| ATOM | 463 | N | PHE | A | 63 | −39.299 | −31.553 | −22.240 | 1.00 | 40.50 | A | N |
| ATOM | 464 | CA | PHE | A | 63 | −38.183 | −31.224 | −21.338 | 1.00 | 39.06 | A | C |
| ATOM | 465 | CB | PHE | A | 63 | −37.901 | −32.388 | −20.376 | 1.00 | 38.22 | A | C |
| ATOM | 466 | CG | PHE | A | 63 | −39.057 | −32.701 | −19.488 | 1.00 | 33.68 | A | C |
| ATOM | 467 | CD1 | PHE | A | 63 | −40.184 | −33.360 | −20.008 | 1.00 | 26.93 | A | C |
| ATOM | 468 | CE1 | PHE | A | 63 | −41.301 | −33.621 | −19.216 | 1.00 | 24.98 | A | C |
| ATOM | 469 | CZ | PHE | A | 63 | −41.285 | −33.253 | −17.871 | 1.00 | 23.12 | A | C |
| ATOM | 470 | CE2 | PHE | A | 63 | −40.143 | −32.585 | −17.311 | 1.00 | 26.49 | A | C |
| ATOM | 471 | CD2 | PHE | A | 63 | −39.044 | −32.300 | −18.132 | 1.00 | 30.53 | A | C |
| ATOM | 472 | C | PHE | A | 63 | −36.977 | −30.840 | −22.146 | 1.00 | 39.10 | A | C |
| ATOM | 473 | O | PHE | A | 63 | −36.152 | −31.698 | −22.538 | 1.00 | 38.84 | A | O |
| ATOM | 474 | N | THR | A | 64 | −36.891 | −29.544 | −22.428 | 1.00 | 38.79 | A | N |
| ATOM | 475 | CA | THR | A | 64 | −35.953 | −29.061 | −23.422 | 1.00 | 38.65 | A | C |
| ATOM | 476 | CB | THR | A | 64 | −36.671 | −28.406 | −24.642 | 1.00 | 39.24 | A | C |
| ATOM | 477 | OG1 | THR | A | 64 | −37.175 | −27.118 | −24.253 | 1.00 | 36.93 | A | O |
| ATOM | 478 | CG2 | THR | A | 64 | −37.812 | −29.295 | −25.262 | 1.00 | 35.51 | A | C |
| ATOM | 479 | C | THR | A | 64 | −34.986 | −28.050 | −22.862 | 1.00 | 39.61 | A | C |
| ATOM | 480 | O | THR | A | 64 | −34.181 | −27.525 | −23.589 | 1.00 | 41.43 | A | O |
| ATOM | 481 | N | THR | A | 65 | −35.033 | −27.741 | −21.586 | 1.00 | 39.94 | A | N |
| ATOM | 482 | CA | THR | A | 65 | −34.115 | −26.719 | −21.101 | 1.00 | 40.84 | A | C |
| ATOM | 483 | CB | THR | A | 65 | −34.622 | −26.076 | −19.777 | 1.00 | 41.39 | A | C |
| ATOM | 484 | OG1 | THR | A | 65 | −34.335 | −26.930 | −18.639 | 1.00 | 45.44 | A | O |
| ATOM | 485 | CG2 | THR | A | 65 | −36.149 | −25.779 | −19.880 | 1.00 | 39.64 | A | C |
| ATOM | 486 | C | THR | A | 65 | −32.716 | −27.304 | −20.962 | 1.00 | 41.43 | A | C |
| ATOM | 487 | O | THR | A | 65 | −32.535 | −28.526 | −20.920 | 1.00 | 42.73 | A | O |
| ATOM | 488 | N | SER | A | 66 | −31.700 | −26.465 | −20.890 | 1.00 | 41.45 | A | N |
| ATOM | 489 | CA | SER | A | 66 | −30.363 | −27.017 | −20.947 | 1.00 | 41.32 | A | C |
| ATOM | 490 | CB | SER | A | 66 | −29.307 | −25.956 | −21.314 | 1.00 | 41.67 | A | C |
| ATOM | 491 | OG | SER | A | 66 | −28.943 | −25.262 | −20.148 | 1.00 | 42.16 | A | O |
| ATOM | 492 | C | SER | A | 66 | −30.011 | −27.800 | −19.658 | 1.00 | 40.64 | A | C |
| ATOM | 493 | O | SER | A | 66 | −29.354 | −28.846 | −19.737 | 1.00 | 41.27 | A | O |
| ATOM | 494 | N | SER | A | 67 | −30.456 | −27.327 | −18.495 | 1.00 | 38.90 | A | N |
| ATOM | 495 | CA | SER | A | 67 | −30.160 | −28.045 | −17.230 | 1.00 | 37.15 | A | C |
| ATOM | 496 | CB | SER | A | 67 | −30.297 | −27.124 | −15.998 | 1.00 | 37.36 | A | C |
| ATOM | 497 | OG | SER | A | 67 | −31.667 | −26.844 | −15.786 | 1.00 | 38.44 | A | O |
| ATOM | 498 | C | SER | A | 67 | −30.986 | −29.349 | −17.077 | 1.00 | 35.34 | A | C |
| ATOM | 499 | O | SER | A | 67 | −30.624 | −30.228 | −16.287 | 1.00 | 33.43 | A | O |
| ATOM | 500 | N | GLN | A | 68 | −32.039 | −29.457 | −17.882 | 1.00 | 33.79 | A | N |
| ATOM | 501 | CA | GLN | A | 68 | −32.809 | −30.662 | −18.027 | 1.00 | 34.91 | A | C |
| ATOM | 502 | CB | GLN | A | 68 | −34.204 | −30.295 | −18.571 | 1.00 | 34.66 | A | C |
| ATOM | 503 | CG | GLN | A | 68 | −35.212 | −29.824 | −17.504 | 1.00 | 38.08 | A | C |
| ATOM | 504 | CD | GLN | A | 68 | −36.542 | −29.280 | −18.091 | 1.00 | 42.37 | A | C |
| ATOM | 505 | OE1 | GLN | A | 68 | −36.660 | −28.995 | −19.300 | 1.00 | 44.88 | A | O |
| ATOM | 506 | NE2 | GLN | A | 68 | −37.549 | −29.160 | −17.230 | 1.00 | 40.98 | A | N |
| ATOM | 507 | C | GLN | A | 68 | −32.221 | −31.796 | −18.942 | 1.00 | 35.05 | A | C |
| ATOM | 508 | O | GLN | A | 68 | −32.826 | −32.901 | −19.060 | 1.00 | 34.80 | A | O |
| ATOM | 509 | N | ARG | A | 69 | −31.125 | −31.505 | −19.661 | 1.00 | 34.05 | A | N |
| ATOM | 510 | CA | ARG | A | 69 | −30.582 | −32.456 | −20.596 | 1.00 | 32.71 | A | C |
| ATOM | 511 | CB | ARG | A | 69 | −29.503 | −31.788 | −21.418 | 1.00 | 33.66 | A | C |
| ATOM | 512 | CG | ARG | A | 69 | −30.046 | −31.047 | −22.706 | 1.00 | 37.81 | A | C |
| ATOM | 513 | CD | ARG | A | 69 | −28.885 | −30.461 | −23.501 | 1.00 | 43.53 | A | C |
| ATOM | 514 | NE | ARG | A | 69 | −29.328 | −29.621 | −24.606 | 1.00 | 49.85 | A | N |
| ATOM | 515 | CZ | ARG | A | 69 | −28.711 | −29.520 | −25.780 | 1.00 | 50.78 | A | C |
| ATOM | 516 | NH1 | ARG | A | 69 | −27.614 | −30.221 | −26.045 | 1.00 | 50.71 | A | N |
| ATOM | 517 | NH2 | ARG | A | 69 | −29.225 | −28.744 | −26.717 | 1.00 | 52.55 | A | N |
| ATOM | 518 | C | ARG | A | 69 | −30.047 | −33.665 | −19.800 | 1.00 | 31.68 | A | C |
| ATOM | 519 | O | ARG | A | 69 | −30.369 | −34.829 | −20.101 | 1.00 | 31.59 | A | O |
| ATOM | 520 | N | ALA | A | 70 | −29.285 | −33.386 | −18.756 | 1.00 | 28.74 | A | N |
| ATOM | 521 | CA | ALA | A | 70 | −28.724 | −34.423 | −17.974 | 1.00 | 27.59 | A | C |
| ATOM | 522 | CB | ALA | A | 70 | −27.785 | −33.868 | −16.899 | 1.00 | 26.50 | A | C |
| ATOM | 523 | C | ALA | A | 70 | −29.855 | −35.274 | −17.389 | 1.00 | 27.63 | A | C |
| ATOM | 524 | O | ALA | A | 70 | −29.716 | −36.512 | −17.323 | 1.00 | 28.16 | A | O |
| ATOM | 525 | N | ALA | A | 71 | −30.997 | −34.655 | −17.048 | 1.00 | 26.34 | A | N |
| ATOM | 526 | CA | ALA | A | 71 | −32.130 | −35.431 | −16.500 | 1.00 | 25.13 | A | C |
| ATOM | 527 | CB | ALA | A | 71 | −33.162 | −34.532 | −15.828 | 1.00 | 26.06 | A | C |
| ATOM | 528 | C | ALA | A | 71 | −32.807 | −36.303 | −17.538 | 1.00 | 24.79 | A | C |
| ATOM | 529 | O | ALA | A | 71 | −33.281 | −37.427 | −17.215 | 1.00 | 25.17 | A | O |
| ATOM | 530 | N | VAL | A | 72 | −32.869 | −35.818 | −18.771 | 1.00 | 23.47 | A | N |
| ATOM | 531 | CA | VAL | A | 72 | −33.480 | −36.593 | −19.829 | 1.00 | 24.72 | A | C |
| ATOM | 532 | CB | VAL | A | 72 | −33.511 | −35.776 | −21.148 | 1.00 | 26.15 | A | C |
| ATOM | 533 | CG1 | VAL | A | 72 | −34.001 | −36.649 | −22.334 | 1.00 | 25.93 | A | C |
| ATOM | 534 | CG2 | VAL | A | 72 | −34.336 | −34.478 | −20.993 | 1.00 | 24.20 | A | C |
| ATOM | 535 | C | VAL | A | 72 | −32.662 | −37.892 | −20.057 | 1.00 | 25.57 | A | C |
| ATOM | 536 | O | VAL | A | 72 | −33.232 | −38.995 | −20.138 | 1.00 | 25.05 | A | O |
| ATOM | 537 | N | ASP | A | 73 | −31.322 | −37.780 | −20.115 | 1.00 | 25.59 | A | N |
| ATOM | 538 | CA | ASP | A | 73 | −30.561 | −38.978 | −20.334 | 1.00 | 25.80 | A | C |
| ATOM | 539 | CB | ASP | A | 73 | −29.082 | −38.733 | −20.575 | 1.00 | 26.54 | A | C |
| ATOM | 540 | CG | ASP | A | 73 | −28.799 | −38.077 | −21.890 | 1.00 | 25.88 | A | C |
| ATOM | 541 | OD1 | ASP | A | 73 | −29.270 | −36.955 | −22.124 | 1.00 | 29.87 | A | O |
| ATOM | 542 | OD2 | ASP | A | 73 | −28.011 | −38.626 | −22.658 | 1.00 | 25.27 | A | O |
| ATOM | 543 | C | ASP | A | 73 | −30.811 | −39.944 | −19.153 | 1.00 | 25.39 | A | C |
| ATOM | 544 | O | ASP | A | 73 | −31.104 | −41.169 | −19.401 | 1.00 | 23.26 | A | O |
| ATOM | 545 | N | ALA | A | 74 | −30.776 | −39.386 | −17.923 | 1.00 | 24.46 | A | N |
| ATOM | 546 | CA | ALA | A | 74 | −30.919 | −40.211 | −16.666 | 1.00 | 24.88 | A | C |
| ATOM | 547 | CB | ALA | A | 74 | −30.708 | −39.411 | −15.371 | 1.00 | 23.92 | A | C |
| ATOM | 548 | C | ALA | A | 74 | −32.261 | −40.865 | −16.648 | 1.00 | 25.23 | A | C |
| ATOM | 549 | O | ALA | A | 74 | −32.355 | −42.051 | −16.430 | 1.00 | 24.99 | A | O |
| ATOM | 550 | N | HIS | A | 75 | −33.305 | −40.100 | −16.967 | 1.00 | 26.40 | A | N |
| ATOM | 551 | CA | HIS | A | 75 | −34.655 | −40.660 | −16.937 | 1.00 | 26.47 | A | C |
| ATOM | 552 | CB | HIS | A | 75 | −35.729 | −39.551 | −17.047 | 1.00 | 25.93 | A | C |
| ATOM | 553 | CG | HIS | A | 75 | −37.106 | −40.028 | −16.728 | 1.00 | 26.63 | A | C |
| ATOM | 554 | ND1 | HIS | A | 75 | −37.570 | −40.152 | −15.432 | 1.00 | 24.12 | A | N |
| ATOM | 555 | CE1 | HIS | A | 75 | −38.815 | −40.607 | −15.458 | 1.00 | 26.83 | A | C |
| ATOM | 556 | NE2 | HIS | A | 75 | −39.178 | −40.775 | −16.720 | 1.00 | 28.65 | A | N |
| ATOM | 557 | CD2 | HIS | A | 75 | −38.119 | −40.438 | −17.535 | 1.00 | 28.72 | A | C |
| ATOM | 558 | C | HIS | A | 75 | −34.817 | −41.735 | −18.031 | 1.00 | 26.15 | A | C |
| ATOM | 559 | O | HIS | A | 75 | −35.248 | −42.830 | −17.754 | 1.00 | 26.75 | A | O |
| ATOM | 560 | N | TYR | A | 76 | −34.435 | −41.418 | −19.262 | 1.00 | 25.17 | A | N |
| ATOM | 561 | CA | TYR | A | 76 | −34.447 | −42.382 | −20.355 | 1.00 | 24.49 | A | C |
| ATOM | 562 | CB | TYR | A | 76 | −34.010 | −41.649 | −21.618 | 1.00 | 25.96 | A | C |
| ATOM | 563 | CG | TYR | A | 76 | −34.184 | −42.446 | −22.878 | 1.00 | 28.99 | A | C |
| ATOM | 564 | CD1 | TYR | A | 76 | −35.399 | −42.486 | −23.521 | 1.00 | 30.72 | A | C |
| ATOM | 565 | CE1 | TYR | A | 76 | −35.575 | −43.211 | −24.728 | 1.00 | 32.22 | A | C |
| ATOM | 566 | CZ | TYR | A | 76 | −34.509 | −43.900 | −25.258 | 1.00 | 35.19 | A | C |
| ATOM | 567 | OH | TYR | A | 76 | −34.691 | −44.631 | −26.403 | 1.00 | 35.34 | A | O |
| ATOM | 568 | CE2 | TYR | A | 76 | −33.273 | −43.884 | −24.614 | 1.00 | 33.11 | A | C |
| ATOM | 569 | CD2 | TYR | A | 76 | −33.110 | −43.170 | −23.432 | 1.00 | 31.42 | A | C |
| ATOM | 570 | C | TYR | A | 76 | −33.562 | −43.611 | −20.148 | 1.00 | 24.10 | A | C |
| ATOM | 571 | O | TYR | A | 76 | −33.976 | −44.755 | −20.350 | 1.00 | 24.86 | A | O |
| ATOM | 572 | N | ASN | A | 77 | −32.325 | −43.420 | −19.724 | 1.00 | 23.91 | A | N |
| ATOM | 573 | CA | ASN | A | 77 | −31.508 | −44.603 | −19.541 | 1.00 | 22.42 | A | C |
| ATOM | 574 | CB | ASN | A | 77 | −30.023 | −44.258 | −19.441 | 1.00 | 22.43 | A | C |
| ATOM | 575 | CG | ASN | A | 77 | −29.463 | −43.749 | −20.739 | 1.00 | 17.76 | A | C |
| ATOM | 576 | OD1 | ASN | A | 77 | −29.953 | −44.040 | −21.828 | 1.00 | 22.44 | A | O |
| ATOM | 577 | ND2 | ASN | A | 77 | −28.526 | −42.890 | −20.620 | 1.00 | 11.56 | A | N |
| ATOM | 578 | C | ASN | A | 77 | −31.943 | −45.543 | −18.424 | 1.00 | 22.81 | A | C |
| ATOM | 579 | O | ASN | A | 77 | −31.979 | −46.757 | −18.642 | 1.00 | 22.77 | A | O |
| ATOM | 580 | N | LEU | A | 78 | −32.249 | −45.009 | −17.242 | 1.00 | 23.23 | A | N |
| ATOM | 581 | CA | LEU | A | 78 | −32.683 | −45.845 | −16.123 | 1.00 | 24.06 | A | C |
| ATOM | 582 | CB | LEU | A | 78 | −32.929 | −44.983 | −14.929 | 1.00 | 24.24 | A | C |
| ATOM | 583 | CG | LEU | A | 78 | −31.961 | −44.886 | −13.740 | 1.00 | 27.66 | A | C |
| ATOM | 584 | CD1 | LEU | A | 78 | −30.668 | −45.654 | −13.841 | 1.00 | 22.27 | A | C |
| ATOM | 585 | CD2 | LEU | A | 78 | −31.740 | −43.409 | −13.364 | 1.00 | 29.07 | A | C |
| ATOM | 586 | C | LEU | A | 78 | −33.950 | −46.645 | −16.492 | 1.00 | 24.84 | A | C |
| ATOM | 587 | O | LEU | A | 78 | −34.119 | −47.808 | −16.078 | 1.00 | 24.42 | A | O |
| ATOM | 588 | N | GLY | A | 79 | −34.816 | −46.050 | −17.321 | 1.00 | 25.35 | A | N |
| ATOM | 589 | CA | GLY | A | 79 | −35.968 | −46.791 | −17.824 | 1.00 | 25.79 | A | C |
| ATOM | 590 | C | GLY | A | 79 | −35.489 | −47.947 | −18.686 | 1.00 | 26.54 | A | C |
| ATOM | 591 | O | GLY | A | 79 | −36.045 | −49.046 | −18.604 | 1.00 | 26.41 | A | O |
| ATOM | 592 | N | LYS | A | 80 | −34.488 | −47.698 | −19.550 | 1.00 | 26.38 | A | N |
| ATOM | 593 | CA | LYS | A | 80 | −33.936 | −48.802 | −20.369 | 1.00 | 27.32 | A | C |
| ATOM | 594 | CB | LYS | A | 80 | −33.011 | −48.302 | −21.504 | 1.00 | 28.49 | A | C |
| ATOM | 595 | CG | LYS | A | 80 | −33.782 | −47.811 | −22.743 | 1.00 | 33.44 | A | C |
| ATOM | 596 | CD | LYS | A | 80 | −33.098 | −48.294 | −24.045 | 1.00 | 42.67 | A | C |
| ATOM | 597 | CE | LYS | A | 80 | −32.247 | −47.154 | −24.682 | 1.00 | 48.39 | A | C |
| ATOM | 598 | NZ | LYS | A | 80 | −32.366 | −47.039 | −26.217 | 1.00 | 49.07 | A | N |
| ATOM | 599 | C | LYS | A | 80 | −33.271 | −49.932 | −19.528 | 1.00 | 25.49 | A | C |
| ATOM | 600 | O | LYS | A | 80 | −33.395 | −51.132 | −19.879 | 1.00 | 25.22 | A | O |
| ATOM | 601 | N | VAL | A | 81 | −32.626 | −49.562 | −18.412 | 1.00 | 23.06 | A | N |
| ATOM | 602 | CA | VAL | A | 81 | −32.000 | −50.552 | −17.577 | 1.00 | 20.88 | A | C |
| ATOM | 603 | CB | VAL | A | 81 | −30.967 | −49.941 | −16.655 | 1.00 | 21.65 | A | C |
| ATOM | 604 | CG1 | VAL | A | 81 | −30.409 | −51.002 | −15.659 | 1.00 | 19.27 | A | C |
| ATOM | 605 | CG2 | VAL | A | 81 | −29.825 | −49.304 | −17.481 | 1.00 | 18.92 | A | C |
| ATOM | 606 | C | VAL | A | 81 | −33.028 | −51.393 | −16.828 | 1.00 | 21.39 | A | C |
| ATOM | 607 | O | VAL | A | 81 | −32.859 | −52.610 | −16.699 | 1.00 | 21.09 | A | O |
| ATOM | 608 | N | TYR | A | 82 | −34.113 | −50.760 | −16.375 | 1.00 | 21.90 | A | N |
| ATOM | 609 | CA | TYR | A | 82 | −35.236 | −51.463 | −15.786 | 1.00 | 21.33 | A | C |
| ATOM | 610 | CB | TYR | A | 82 | −36.373 | −50.475 | −15.331 | 1.00 | 22.04 | A | C |
| ATOM | 611 | CG | TYR | A | 82 | −37.629 | −51.250 | −14.970 | 1.00 | 18.84 | A | C |
| ATOM | 612 | CD1 | TYR | A | 82 | −38.503 | −51.665 | −15.990 | 1.00 | 19.21 | A | C |
| ATOM | 613 | CE1 | TYR | A | 82 | −39.585 | −52.444 | −15.776 | 1.00 | 18.52 | A | C |
| ATOM | 614 | CZ | TYR | A | 82 | −39.883 | −52.874 | −14.525 | 1.00 | 21.16 | A | C |
| ATOM | 615 | OH | TYR | A | 82 | −41.020 | −53.656 | −14.432 | 1.00 | 24.42 | A | O |
| ATOM | 616 | CE2 | TYR | A | 82 | −39.062 | −52.536 | −13.457 | 1.00 | 20.05 | A | C |
| ATOM | 617 | CD2 | TYR | A | 82 | −37.855 | −51.701 | −13.722 | 1.00 | 16.33 | A | C |
| ATOM | 618 | C | TYR | A | 82 | −35.781 | −52.476 | −16.808 | 1.00 | 22.26 | A | C |
| ATOM | 619 | O | TYR | A | 82 | −36.007 | −53.649 | −16.479 | 1.00 | 22.32 | A | O |
| ATOM | 620 | N | ASP | A | 83 | −36.041 | −52.029 | −18.030 | 1.00 | 22.20 | A | N |
| ATOM | 621 | CA | ASP | A | 83 | −36.498 | −52.971 | −19.075 | 1.00 | 23.53 | A | C |
| ATOM | 622 | CB | ASP | A | 83 | −36.745 | −52.268 | −20.416 | 1.00 | 23.02 | A | C |
| ATOM | 623 | CG | ASP | A | 83 | −37.831 | −51.196 | −20.317 | 1.00 | 25.23 | A | C |
| ATOM | 624 | OD1 | ASP | A | 83 | −38.713 | −51.279 | −19.401 | 1.00 | 24.63 | A | O |
| ATOM | 625 | OD2 | ASP | A | 83 | −37.799 | −50.280 | −21.163 | 1.00 | 28.39 | A | O |
| ATOM | 626 | C | ASP | A | 83 | −35.555 | −54.134 | −19.318 | 1.00 | 23.48 | A | C |
| ATOM | 627 | O | ASP | A | 83 | −35.971 | −55.249 | −19.589 | 1.00 | 23.92 | A | O |
| ATOM | 628 | N | TYR | A | 84 | −34.271 | −53.874 | −19.216 | 1.00 | 24.00 | A | N |
| ATOM | 629 | CA | TYR | A | 84 | −33.326 | −54.902 | −19.519 | 1.00 | 23.71 | A | C |
| ATOM | 630 | CB | TYR | A | 84 | −31.907 | −54.297 | −19.583 | 1.00 | 24.20 | A | C |
| ATOM | 631 | CG | TYR | A | 84 | −30.858 | −55.363 | −19.629 | 1.00 | 23.19 | A | C |
| ATOM | 632 | CD1 | TYR | A | 84 | −30.519 | −55.931 | −20.823 | 1.00 | 22.87 | A | C |
| ATOM | 633 | CE1 | TYR | A | 84 | −29.551 | −56.947 | −20.875 | 1.00 | 24.91 | A | C |
| ATOM | 634 | CZ | TYR | A | 84 | −28.992 | −57.421 | −19.717 | 1.00 | 20.31 | A | C |
| ATOM | 635 | OH | TYR | A | 84 | −28.075 | −58.443 | −19.820 | 1.00 | 22.01 | A | O |
| ATOM | 636 | CE2 | TYR | A | 84 | −29.342 | −56.869 | −18.521 | 1.00 | 19.40 | A | C |
| ATOM | 637 | CD2 | TYR | A | 84 | −30.252 | −55.840 | −18.465 | 1.00 | 18.29 | A | C |
| ATOM | 638 | C | TYR | A | 84 | −33.437 | −56.034 | −18.501 | 1.00 | 23.26 | A | C |
| ATOM | 639 | O | TYR | A | 84 | −33.541 | −57.178 | −18.881 | 1.00 | 24.20 | A | O |
| ATOM | 640 | N | PHE | A | 85 | −33.418 | −55.715 | −17.218 | 1.00 | 22.52 | A | N |
| ATOM | 641 | CA | PHE | A | 85 | −33.429 | −56.712 | −16.173 | 1.00 | 22.03 | A | C |
| ATOM | 642 | CB | PHE | A | 85 | −33.030 | −56.067 | −14.835 | 1.00 | 22.20 | A | C |
| ATOM | 643 | CG | PHE | A | 85 | −31.531 | −55.940 | −14.665 | 1.00 | 20.97 | A | C |
| ATOM | 644 | CD1 | PHE | A | 85 | −30.756 | −57.049 | −14.344 | 1.00 | 18.41 | A | C |
| ATOM | 645 | CE1 | PHE | A | 85 | −29.382 | −56.971 | −14.210 | 1.00 | 17.21 | A | C |
| ATOM | 646 | CZ | PHE | A | 85 | −28.735 | −55.766 | −14.418 | 1.00 | 20.03 | A | C |
| ATOM | 647 | CE2 | PHE | A | 85 | −29.477 | −54.653 | −14.776 | 1.00 | 21.83 | A | C |
| ATOM | 648 | CD2 | PHE | A | 85 | −30.892 | −54.745 | −14.895 | 1.00 | 23.45 | A | C |
| ATOM | 649 | C | PHE | A | 85 | −34.781 | −57.390 | −16.087 | 1.00 | 23.25 | A | C |
| ATOM | 650 | O | PHE | A | 85 | −34.867 | −58.629 | −15.837 | 1.00 | 21.25 | A | O |
| ATOM | 651 | N | TYR | A | 86 | −35.831 | −56.599 | −16.381 | 1.00 | 23.68 | A | N |
| ATOM | 652 | CA | TYR | A | 86 | −37.186 | −57.146 | −16.398 | 1.00 | 24.50 | A | C |
| ATOM | 653 | CB | TYR | A | 86 | −38.259 | −56.060 | −16.341 | 1.00 | 25.13 | A | C |
| ATOM | 654 | CG | TYR | A | 86 | −39.648 | −56.640 | −16.311 | 1.00 | 26.50 | A | C |
| ATOM | 655 | CD1 | TYR | A | 86 | −40.122 | −57.303 | −15.169 | 1.00 | 27.15 | A | C |
| ATOM | 656 | CE1 | TYR | A | 86 | −41.398 | −57.867 | −15.148 | 1.00 | 29.74 | A | C |
| ATOM | 657 | CZ | TYR | A | 86 | −42.220 | −57.763 | −16.274 | 1.00 | 30.75 | A | C |
| ATOM | 658 | OH | TYR | A | 86 | −43.463 | −58.290 | −16.229 | 1.00 | 33.52 | A | O |
| ATOM | 659 | CE2 | TYR | A | 86 | −41.800 | −57.146 | −17.414 | 1.00 | 29.62 | A | C |
| ATOM | 660 | CD2 | TYR | A | 86 | −40.477 | −56.574 | −17.429 | 1.00 | 29.65 | A | C |
| ATOM | 661 | C | TYR | A | 86 | −37.438 | −58.080 | −17.562 | 1.00 | 24.44 | A | C |
| ATOM | 662 | O | TYR | A | 86 | −37.906 | −59.199 | −17.381 | 1.00 | 25.04 | A | O |
| ATOM | 663 | N | GLN | A | 87 | −37.088 | −57.657 | −18.754 | 1.00 | 24.92 | A | N |
| ATOM | 664 | CA | GLN | A | 87 | −37.333 | −58.504 | −19.925 | 1.00 | 26.25 | A | C |
| ATOM | 665 | CB | GLN | A | 87 | −37.232 | −57.687 | −21.197 | 1.00 | 26.93 | A | C |
| ATOM | 666 | CG | GLN | A | 87 | −38.411 | −56.771 | −21.381 | 1.00 | 31.10 | A | C |
| ATOM | 667 | CD | GLN | A | 87 | −38.085 | −55.660 | −22.348 | 1.00 | 39.99 | A | C |
| ATOM | 668 | OE1 | GLN | A | 87 | −37.081 | −55.726 | −23.071 | 1.00 | 41.78 | A | O |
| ATOM | 669 | NE2 | GLN | A | 87 | −38.922 | −54.617 | −22.366 | 1.00 | 42.63 | A | N |
| ATOM | 670 | C | GLN | A | 87 | −36.420 | −59.706 | −20.067 | 1.00 | 26.15 | A | C |
| ATOM | 671 | O | GLN | A | 87 | −36.831 | −60.699 | −20.644 | 1.00 | 24.48 | A | O |
| ATOM | 672 | N | LYS | A | 88 | −35.153 | −59.593 | −19.620 | 1.00 | 25.63 | A | N |
| ATOM | 673 | CA | LYS | A | 88 | −34.252 | −60.725 | −19.772 | 1.00 | 24.20 | A | C |
| ATOM | 674 | CB | LYS | A | 88 | −32.770 | −60.326 | −19.777 | 1.00 | 24.42 | A | C |
| ATOM | 675 | CG | LYS | A | 88 | −32.352 | −59.286 | −20.748 | 1.00 | 24.40 | A | C |
| ATOM | 676 | CD | LYS | A | 88 | −32.523 | −59.738 | −22.116 | 1.00 | 26.90 | A | C |
| ATOM | 677 | CE | LYS | A | 88 | −32.183 | −58.598 | −23.050 | 1.00 | 27.59 | A | C |
| ATOM | 678 | NZ | LYS | A | 88 | −31.931 | −59.174 | −24.395 | 1.00 | 31.00 | A | N |
| ATOM | 679 | C | LYS | A | 88 | −34.457 | −61.711 | −18.670 | 1.00 | 23.70 | A | C |
| ATOM | 680 | O | LYS | A | 88 | −34.342 | −62.871 | −18.919 | 1.00 | 24.35 | A | O |
| ATOM | 681 | N | PHE | A | 89 | −34.692 | −61.267 | −17.440 | 1.00 | 23.60 | A | N |
| ATOM | 682 | CA | PHE | A | 89 | −34.576 | −62.182 | −16.311 | 1.00 | 23.45 | A | C |
| ATOM | 683 | CB | PHE | A | 89 | −33.397 | −61.806 | −15.422 | 1.00 | 22.33 | A | C |
| ATOM | 684 | CG | PHE | A | 89 | −32.147 | −61.509 | −16.191 | 1.00 | 23.28 | A | C |
| ATOM | 685 | CD1 | PHE | A | 89 | −31.498 | −62.518 | −16.917 | 1.00 | 19.53 | A | C |
| ATOM | 686 | CE1 | PHE | A | 89 | −30.332 | −62.222 | −17.674 | 1.00 | 21.34 | A | C |
| ATOM | 687 | CZ | PHE | A | 89 | −29.809 | −60.916 | −17.709 | 1.00 | 20.21 | A | C |
| ATOM | 688 | CE2 | PHE | A | 89 | −30.462 | −59.892 | −16.997 | 1.00 | 21.28 | A | C |
| ATOM | 689 | CD2 | PHE | A | 89 | −31.634 | −60.190 | −16.251 | 1.00 | 21.81 | A | C |
| ATOM | 690 | C | PHE | A | 89 | −35.845 | −62.205 | −15.497 | 1.00 | 24.67 | A | C |
| ATOM | 691 | O | PHE | A | 89 | −35.910 | −62.900 | −14.489 | 1.00 | 23.59 | A | O |
| ATOM | 692 | N | ASN | A | 90 | −36.848 | −61.422 | −15.927 | 1.00 | 25.75 | A | N |
| ATOM | 693 | CA | ASN | A | 90 | −38.049 | −61.227 | −15.121 | 1.00 | 26.46 | A | C |
| ATOM | 694 | CB | ASN | A | 90 | −38.887 | −62.559 | −15.076 | 1.00 | 27.00 | A | C |
| ATOM | 695 | CG | ASN | A | 90 | −40.308 | −62.371 | −14.453 | 1.00 | 30.20 | A | C |
| ATOM | 696 | OD1 | ASN | A | 90 | −41.067 | −61.430 | −14.783 | 1.00 | 32.17 | A | O |
| ATOM | 697 | ND2 | ASN | A | 90 | −40.664 | −63.284 | −13.556 | 1.00 | 29.30 | A | N |
| ATOM | 698 | C | ASN | A | 90 | −37.691 | −60.676 | −13.725 | 1.00 | 25.55 | A | C |
| ATOM | 699 | O | ASN | A | 90 | −38.285 | −61.086 | −12.707 | 1.00 | 27.83 | A | O |
| ATOM | 700 | N | ARG | A | 91 | −36.740 | −59.751 | −13.662 | 1.00 | 24.18 | A | N |
| ATOM | 701 | CA | ARG | A | 91 | −36.398 | −59.079 | −12.383 | 1.00 | 24.35 | A | C |
| ATOM | 702 | CB | ARG | A | 91 | −34.894 | −58.713 | −12.244 | 1.00 | 24.28 | A | C |
| ATOM | 703 | CG | ARG | A | 91 | −34.459 | −58.341 | −10.821 | 1.00 | 20.60 | A | C |
| ATOM | 704 | CD | ARG | A | 91 | −32.985 | −58.061 | −10.691 | 1.00 | 19.78 | A | C |
| ATOM | 705 | NE | ARG | A | 91 | −32.656 | −57.679 | −9.324 | 1.00 | 16.07 | A | N |
| ATOM | 706 | CZ | ARG | A | 91 | −32.448 | −58.597 | −8.355 | 1.00 | 20.75 | A | C |
| ATOM | 707 | NH1 | ARG | A | 91 | −32.544 | −59.918 | −8.634 | 1.00 | 14.39 | A | N |
| ATOM | 708 | NH2 | ARG | A | 91 | −32.194 | −58.219 | −7.091 | 1.00 | 16.44 | A | N |
| ATOM | 709 | C | ARG | A | 91 | −37.210 | −57.826 | −12.208 | 1.00 | 24.66 | A | C |
| ATOM | 710 | O | ARG | A | 91 | −37.210 | −56.943 | −13.073 | 1.00 | 26.57 | A | O |
| ATOM | 711 | N | ASN | A | 92 | −37.924 | −57.722 | −11.102 | 1.00 | 24.72 | A | N |
| ATOM | 712 | CA | ASN | A | 92 | −38.747 | −56.532 | −10.900 | 1.00 | 24.42 | A | C |
| ATOM | 713 | CB | ASN | A | 92 | −39.951 | −56.942 | −10.084 | 1.00 | 24.43 | A | C |
| ATOM | 714 | CG | ASN | A | 92 | −40.921 | −55.822 | −9.831 | 1.00 | 27.29 | A | C |
| ATOM | 715 | OD1 | ASN | A | 92 | −40.874 | −54.757 | −10.450 | 1.00 | 30.10 | A | O |
| ATOM | 716 | ND2 | ASN | A | 92 | −41.866 | −56.084 | −8.913 | 1.00 | 29.96 | A | N |
| ATOM | 717 | C | ASN | A | 92 | −37.915 | −55.455 | −10.201 | 1.00 | 24.37 | A | C |
| ATOM | 718 | O | ASN | A | 92 | −37.937 | −55.333 | −8.965 | 1.00 | 25.59 | A | O |
| ATOM | 719 | N | SER | A | 93 | −37.175 | −54.673 | −10.970 | 1.00 | 22.83 | A | N |
| ATOM | 720 | CA | SER | A | 93 | −36.336 | −53.650 | −10.387 | 1.00 | 22.27 | A | C |
| ATOM | 721 | CB | SER | A | 93 | −37.090 | −52.772 | −9.384 | 1.00 | 21.81 | A | C |
| ATOM | 722 | OG | SER | A | 93 | −36.335 | −51.571 | −9.193 | 1.00 | 20.51 | A | O |
| ATOM | 723 | C | SER | A | 93 | −35.081 | −54.191 | −9.730 | 1.00 | 22.08 | A | C |
| ATOM | 724 | O | SER | A | 93 | −34.886 | −55.378 | −9.638 | 1.00 | 23.02 | A | O |
| ATOM | 725 | N | TYR | A | 94 | −34.213 | −53.303 | −9.254 | 1.00 | 23.13 | A | N |
| ATOM | 726 | CA | TYR | A | 94 | −32.915 | −53.741 | −8.709 | 1.00 | 22.74 | A | C |
| ATOM | 727 | CB | TYR | A | 94 | −32.040 | −52.547 | −8.387 | 1.00 | 21.69 | A | C |
| ATOM | 728 | CG | TYR | A | 94 | −32.522 | −51.788 | −7.184 | 1.00 | 21.77 | A | C |
| ATOM | 729 | CD1 | TYR | A | 94 | −31.986 | −52.046 | −5.904 | 1.00 | 25.43 | A | C |
| ATOM | 730 | CE1 | TYR | A | 94 | −32.444 | −51.362 | −4.749 | 1.00 | 21.99 | A | C |
| ATOM | 731 | CZ | TYR | A | 94 | −33.460 | −50.434 | −4.881 | 1.00 | 24.51 | A | C |
| ATOM | 732 | OH | TYR | A | 94 | −33.935 | −49.748 | −3.758 | 1.00 | 24.58 | A | O |
| ATOM | 733 | CE2 | TYR | A | 94 | −34.028 | −50.186 | −6.162 | 1.00 | 23.54 | A | C |
| ATOM | 734 | CD2 | TYR | A | 94 | −33.558 | −50.857 | −7.288 | 1.00 | 18.74 | A | C |
| ATOM | 735 | C | TYR | A | 94 | −33.034 | −54.650 | −7.501 | 1.00 | 23.34 | A | C |
| ATOM | 736 | O | TYR | A | 94 | −32.184 | −55.516 | −7.295 | 1.00 | 25.03 | A | O |
| ATOM | 737 | N | ASP | A | 95 | −34.077 | −54.502 | −6.699 | 1.00 | 24.79 | A | N |
| ATOM | 738 | CA | ASP | A | 95 | −34.229 | −55.405 | −5.504 | 1.00 | 25.96 | A | C |
| ATOM | 739 | CB | ASP | A | 95 | −34.718 | −54.618 | −4.299 | 1.00 | 25.17 | A | C |
| ATOM | 740 | CG | ASP | A | 95 | −36.080 | −53.926 | −4.563 | 1.00 | 26.88 | A | C |
| ATOM | 741 | OD1 | ASP | A | 95 | −36.708 | −54.173 | −5.621 | 1.00 | 23.59 | A | O |
| ATOM | 742 | OD2 | ASP | A | 95 | −36.526 | −53.122 | −3.710 | 1.00 | 30.47 | A | O |
| ATOM | 743 | C | ASP | A | 95 | −35.186 | −56.550 | −5.756 | 1.00 | 26.31 | A | C |
| ATOM | 744 | O | ASP | A | 95 | −35.570 | −57.253 | −4.830 | 1.00 | 28.26 | A | O |
| ATOM | 745 | N | ASN | A | 96 | −35.622 | −56.705 | −6.996 | 1.00 | 26.19 | A | N |
| ATOM | 746 | CA | ASN | A | 96 | −36.600 | −57.726 | −7.345 | 1.00 | 27.04 | A | C |
| ATOM | 747 | CB | ASN | A | 96 | −35.994 | −59.141 | −7.290 | 1.00 | 25.92 | A | C |
| ATOM | 748 | CG | ASN | A | 96 | −36.783 | −60.126 | −8.133 | 1.00 | 27.06 | A | C |
| ATOM | 749 | OD1 | ASN | A | 96 | −37.509 | −59.722 | −9.059 | 1.00 | 28.41 | A | O |
| ATOM | 750 | ND2 | ASN | A | 96 | −36.649 | −61.414 | −7.833 | 1.00 | 23.23 | A | N |
| ATOM | 751 | C | ASN | A | 96 | −37.868 | −57.680 | −6.491 | 1.00 | 27.67 | A | C |
| ATOM | 752 | O | ASN | A | 96 | −38.536 | −58.706 | −6.354 | 1.00 | 27.62 | A | O |
| ATOM | 753 | N | LYS | A | 97 | −38.169 | −56.506 | −5.911 | 1.00 | 28.43 | A | N |
| ATOM | 754 | CA | LYS | A | 97 | −39.373 | −56.260 | −5.095 | 1.00 | 27.97 | A | C |
| ATOM | 755 | CB | LYS | A | 97 | −39.009 | −56.065 | −3.606 | 1.00 | 28.01 | A | C |
| ATOM | 756 | CG | LYS | A | 97 | −38.492 | −57.348 | −2.937 | 1.00 | 29.56 | A | C |
| ATOM | 757 | CD | LYS | A | 97 | −38.148 | −57.180 | −1.435 | 1.00 | 31.10 | A | C |
| ATOM | 758 | CE | LYS | A | 97 | −36.711 | −56.724 | −1.178 | 1.00 | 32.30 | A | C |
| ATOM | 759 | NZ | LYS | A | 97 | −36.314 | −56.990 | 0.232 | 1.00 | 35.76 | A | N |
| ATOM | 760 | C | LYS | A | 97 | −40.134 | −55.048 | −5.613 | 1.00 | 28.14 | A | C |
| ATOM | 761 | O | LYS | A | 97 | −41.135 | −54.648 | −5.017 | 1.00 | 28.53 | A | O |
| ATOM | 762 | N | GLY | A | 98 | −39.669 | −54.447 | −6.711 | 1.00 | 27.15 | A | N |
| ATOM | 763 | CA | GLY | A | 98 | −40.370 | −53.299 | −7.301 | 1.00 | 24.49 | A | C |
| ATOM | 764 | C | GLY | A | 98 | −39.850 | −51.961 | −6.793 | 1.00 | 23.46 | A | C |
| ATOM | 765 | O | GLY | A | 98 | −40.445 | −50.935 | −7.082 | 1.00 | 24.40 | A | O |
| ATOM | 766 | N | GLY | A | 99 | −38.776 | −51.947 | −6.013 | 1.00 | 21.47 | A | N |
| ATOM | 767 | CA | GLY | A | 99 | −38.290 | −50.710 | −5.411 | 1.00 | 20.97 | A | C |
| ATOM | 768 | C | GLY | A | 99 | −38.114 | −49.600 | −6.437 | 1.00 | 23.26 | A | C |
| ATOM | 769 | O | GLY | A | 99 | −37.507 | −49.813 | −7.495 | 1.00 | 22.85 | A | O |
| ATOM | 770 | N | LYS | A | 100 | −38.678 | −48.408 | −6.186 | 1.00 | 24.99 | A | N |
| ATOM | 771 | CA | LYS | A | 100 | −38.545 | −47.281 | −7.141 | 1.00 | 25.11 | A | C |
| ATOM | 772 | CB | LYS | A | 100 | −39.214 | −46.034 | −6.577 | 1.00 | 25.69 | A | C |
| ATOM | 773 | CG | LYS | A | 100 | −38.639 | −45.629 | −5.203 | 1.00 | 30.14 | A | C |
| ATOM | 774 | CD | LYS | A | 100 | −39.052 | −44.248 | −4.642 | 1.00 | 32.29 | A | C |
| ATOM | 775 | CE | LYS | A | 100 | −38.162 | −43.991 | −3.336 | 1.00 | 38.39 | A | C |
| ATOM | 776 | NZ | LYS | A | 100 | −38.707 | −42.859 | −2.443 | 1.00 | 42.60 | A | N |
| ATOM | 777 | C | LYS | A | 100 | −37.036 | −47.043 | −7.447 | 1.00 | 25.30 | A | C |
| ATOM | 778 | O | LYS | A | 100 | −36.150 | −47.345 | −6.610 | 1.00 | 24.91 | A | O |
| ATOM | 779 | N | ILE | A | 101 | −36.761 | −46.564 | −8.663 | 1.00 | 24.85 | A | N |
| ATOM | 780 | CA | ILE | A | 101 | −35.414 | −46.283 | −9.161 | 1.00 | 23.22 | A | C |
| ATOM | 781 | CB | ILE | A | 101 | −35.255 | −46.923 | −10.522 | 1.00 | 23.11 | A | C |
| ATOM | 782 | CG1 | ILE | A | 101 | −35.219 | −48.449 | −10.364 | 1.00 | 20.36 | A | C |
| ATOM | 783 | CD1 | ILE | A | 101 | −35.453 | −49.172 | −11.638 | 1.00 | 21.07 | A | C |
| ATOM | 784 | CG2 | ILE | A | 101 | −33.999 | −46.362 | −11.287 | 1.00 | 23.99 | A | C |
| ATOM | 785 | C | ILE | A | 101 | −35.288 | −44.776 | −9.290 | 1.00 | 24.21 | A | C |
| ATOM | 786 | O | ILE | A | 101 | −35.944 | −44.171 | −10.146 | 1.00 | 26.06 | A | O |
| ATOM | 787 | N | VAL | A | 102 | −34.503 | −44.153 | −8.421 | 1.00 | 23.16 | A | N |
| ATOM | 788 | CA | VAL | A | 102 | −34.478 | −42.727 | −8.287 | 1.00 | 22.20 | A | C |
| ATOM | 789 | CB | VAL | A | 102 | −34.761 | −42.340 | −6.807 | 1.00 | 23.44 | A | C |
| ATOM | 790 | CG1 | VAL | A | 102 | −34.424 | −40.850 | −6.483 | 1.00 | 19.05 | A | C |
| ATOM | 791 | CG2 | VAL | A | 102 | −36.180 | −42.671 | −6.460 | 1.00 | 21.14 | A | C |
| ATOM | 792 | C | VAL | A | 102 | −33.084 | −42.267 | −8.677 | 1.00 | 23.93 | A | C |
| ATOM | 793 | O | VAL | A | 102 | −32.091 | −42.941 | −8.325 | 1.00 | 22.72 | A | O |
| ATOM | 794 | N | SER | A | 103 | −32.994 | −41.148 | −9.421 | 1.00 | 24.36 | A | N |
| ATOM | 795 | CA | SER | A | 103 | −31.708 | −40.568 | −9.703 | 1.00 | 25.59 | A | C |
| ATOM | 796 | CB | SER | A | 103 | −31.317 | −40.856 | −11.124 | 1.00 | 25.68 | A | C |
| ATOM | 797 | OG | SER | A | 103 | −31.926 | −39.882 | −11.946 | 1.00 | 28.96 | A | O |
| ATOM | 798 | C | SER | A | 103 | −31.654 | −39.047 | −9.411 | 1.00 | 26.51 | A | C |
| ATOM | 799 | O | SER | A | 103 | −32.695 | −38.350 | −9.383 | 1.00 | 26.56 | A | O |
| ATOM | 800 | N | SER | A | 104 | −30.442 | −38.556 | −9.140 | 1.00 | 26.44 | A | N |
| ATOM | 801 | CA | SER | A | 104 | −30.225 | −37.140 | −8.958 | 1.00 | 26.76 | A | C |
| ATOM | 802 | CB | SER | A | 104 | −29.852 | −36.784 | −7.518 | 1.00 | 27.23 | A | C |
| ATOM | 803 | OG | SER | A | 104 | −30.856 | −37.188 | −6.574 | 1.00 | 26.06 | A | O |
| ATOM | 804 | C | SER | A | 104 | −29.123 | −36.728 | −9.918 | 1.00 | 27.68 | A | C |
| ATOM | 805 | O | SER | A | 104 | −27.979 | −37.206 | −9.868 | 1.00 | 27.25 | A | O |
| ATOM | 806 | N | VAL | A | 105 | −29.495 | −35.861 | −10.841 | 1.00 | 28.11 | A | N |
| ATOM | 807 | CA | VAL | A | 105 | −28.530 | −35.248 | −11.711 | 1.00 | 28.88 | A | C |
| ATOM | 808 | CB | VAL | A | 105 | −29.152 | −35.040 | −13.083 | 1.00 | 29.18 | A | C |
| ATOM | 809 | CG1 | VAL | A | 105 | −29.569 | −36.358 | −13.662 | 1.00 | 27.85 | A | C |
| ATOM | 810 | CG2 | VAL | A | 105 | −30.338 | −34.066 | −12.990 | 1.00 | 28.33 | A | C |
| ATOM | 811 | C | VAL | A | 105 | −28.034 | −33.904 | −11.141 | 1.00 | 29.60 | A | C |
| ATOM | 812 | O | VAL | A | 105 | −28.611 | −33.353 | −10.151 | 1.00 | 29.63 | A | O |
| ATOM | 813 | N | HIS | A | 106 | −27.005 | −33.356 | −11.782 | 1.00 | 29.73 | A | N |
| ATOM | 814 | CA | HIS | A | 106 | −26.377 | −32.102 | −11.318 | 1.00 | 30.74 | A | C |
| ATOM | 815 | CB | HIS | A | 106 | −27.287 | −30.869 | −11.536 | 1.00 | 30.34 | A | C |
| ATOM | 816 | CG | HIS | A | 106 | −27.705 | −30.693 | −12.961 | 1.00 | 27.61 | A | C |
| ATOM | 817 | ND1 | HIS | A | 106 | −26.794 | −30.645 | −13.990 | 1.00 | 25.34 | A | N |
| ATOM | 818 | CE1 | HIS | A | 106 | −27.437 | −30.525 | −15.140 | 1.00 | 28.07 | A | C |
| ATOM | 819 | NE2 | HIS | A | 106 | −28.732 | −30.482 | −14.893 | 1.00 | 28.43 | A | N |
| ATOM | 820 | CD2 | HIS | A | 106 | −28.922 | −30.593 | −13.532 | 1.00 | 28.46 | A | C |
| ATOM | 821 | C | HIS | A | 106 | −25.971 | −32.252 | −9.860 | 1.00 | 32.36 | A | C |
| ATOM | 822 | O | HIS | A | 106 | −26.131 | −31.330 | −9.040 | 1.00 | 32.69 | A | O |
| ATOM | 823 | N | TYR | A | 107 | −25.461 | −33.433 | −9.528 | 1.00 | 33.47 | A | N |
| ATOM | 824 | CA | TYR | A | 107 | −24.924 | −33.638 | −8.211 | 1.00 | 35.05 | A | C |
| ATOM | 825 | CB | TYR | A | 107 | −24.727 | −35.116 | −7.926 | 1.00 | 34.59 | A | C |
| ATOM | 826 | CG | TYR | A | 107 | −24.107 | −35.405 | −6.575 | 1.00 | 35.69 | A | C |
| ATOM | 827 | CD1 | TYR | A | 107 | −24.910 | −35.735 | −5.483 | 1.00 | 35.84 | A | C |
| ATOM | 828 | CE1 | TYR | A | 107 | −24.339 | −36.017 | −4.221 | 1.00 | 36.98 | A | C |
| ATOM | 829 | CZ | TYR | A | 107 | −22.964 | −35.989 | −4.073 | 1.00 | 37.15 | A | C |
| ATOM | 830 | OH | TYR | A | 107 | −22.457 | −36.265 | −2.851 | 1.00 | 37.25 | A | O |
| ATOM | 831 | CE2 | TYR | A | 107 | −22.126 | −35.689 | −5.149 | 1.00 | 36.54 | A | C |
| ATOM | 832 | CD2 | TYR | A | 107 | −22.701 | −35.393 | −6.393 | 1.00 | 35.92 | A | C |
| ATOM | 833 | C | TYR | A | 107 | −23.598 | −32.924 | −8.176 | 1.00 | 36.00 | A | C |
| ATOM | 834 | O | TYR | A | 107 | −22.712 | −33.209 | −9.022 | 1.00 | 35.53 | A | O |
| ATOM | 835 | N | GLY | A | 108 | −23.467 | −32.001 | −7.210 | 1.00 | 37.18 | A | N |
| ATOM | 836 | CA | GLY | A | 108 | −22.178 | −31.379 | −6.893 | 1.00 | 38.18 | A | C |
| ATOM | 837 | C | GLY | A | 108 | −21.753 | −30.343 | −7.914 | 1.00 | 40.33 | A | C |
| ATOM | 838 | O | GLY | A | 108 | −22.577 | −29.768 | −8.643 | 1.00 | 40.64 | A | O |
| ATOM | 839 | N | SER | A | 109 | −20.457 | −30.086 | −7.969 | 1.00 | 42.15 | A | N |
| ATOM | 840 | CA | SER | A | 109 | −19.971 | −29.038 | −8.853 | 1.00 | 44.71 | A | C |
| ATOM | 841 | CB | SER | A | 109 | −19.611 | −27.751 | −8.074 | 1.00 | 45.07 | A | C |
| ATOM | 842 | OG | SER | A | 109 | −19.609 | −26.644 | −8.968 | 1.00 | 45.28 | A | O |
| ATOM | 843 | C | SER | A | 109 | −18.768 | −29.559 | −9.638 | 1.00 | 45.55 | A | C |
| ATOM | 844 | O | SER | A | 109 | −17.772 | −30.016 | −9.026 | 1.00 | 45.22 | A | O |
| ATOM | 845 | N | ARG | A | 110 | −18.890 | −29.541 | −10.977 | 1.00 | 45.44 | A | N |
| ATOM | 846 | CA | ARG | A | 110 | −17.854 | −30.119 | −11.821 | 1.00 | 46.27 | A | C |
| ATOM | 847 | CB | ARG | A | 110 | −16.732 | −29.064 | −11.996 | 1.00 | 47.09 | A | C |
| ATOM | 848 | CG | ARG | A | 110 | −17.316 | −27.667 | −12.469 | 1.00 | 53.17 | A | C |
| ATOM | 849 | CD | ARG | A | 110 | −16.356 | −26.720 | −13.257 | 1.00 | 61.65 | A | C |
| ATOM | 850 | NE | ARG | A | 110 | −14.954 | −26.807 | −12.807 | 1.00 | 67.59 | A | N |
| ATOM | 851 | CZ | ARG | A | 110 | −13.906 | −27.184 | −13.564 | 1.00 | 70.42 | A | C |
| ATOM | 852 | NH1 | ARG | A | 110 | −14.028 | −27.510 | −14.865 | 1.00 | 69.00 | A | N |
| ATOM | 853 | NH2 | ARG | A | 110 | −12.704 | −27.241 | −13.006 | 1.00 | 71.16 | A | N |
| ATOM | 854 | C | ARG | A | 110 | −17.354 | −31.497 | −11.255 | 1.00 | 44.76 | A | C |
| ATOM | 855 | O | ARG | A | 110 | −16.167 | −31.772 | −11.201 | 1.00 | 44.86 | A | O |
| ATOM | 856 | N | TYR | A | 111 | −18.284 | −32.349 | −10.816 | 1.00 | 43.15 | A | N |
| ATOM | 857 | CA | TYR | A | 111 | −17.967 | −33.613 | −10.113 | 1.00 | 41.46 | A | C |
| ATOM | 858 | CB | TYR | A | 111 | −19.129 | −33.916 | −9.167 | 1.00 | 41.36 | A | C |
| ATOM | 859 | CG | TYR | A | 111 | −19.025 | −35.147 | −8.321 | 1.00 | 43.63 | A | C |
| ATOM | 860 | CD1 | TYR | A | 111 | −17.981 | −35.319 | −7.403 | 1.00 | 46.38 | A | C |
| ATOM | 861 | CE1 | TYR | A | 111 | −17.907 | −36.482 | −6.593 | 1.00 | 48.54 | A | C |
| ATOM | 862 | CZ | TYR | A | 111 | −18.895 | −37.465 | −6.693 | 1.00 | 49.57 | A | C |
| ATOM | 863 | OH | TYR | A | 111 | −18.842 | −38.598 | −5.909 | 1.00 | 50.97 | A | O |
| ATOM | 864 | CE2 | TYR | A | 111 | −19.946 | −37.312 | −7.594 | 1.00 | 48.80 | A | C |
| ATOM | 865 | CD2 | TYR | A | 111 | −20.004 | −36.152 | −8.405 | 1.00 | 47.79 | A | C |
| ATOM | 866 | C | TYR | A | 111 | −17.735 | −34.788 | −11.088 | 1.00 | 40.02 | A | C |
| ATOM | 867 | O | TYR | A | 111 | −18.580 | −35.098 | −11.906 | 1.00 | 38.85 | A | O |
| ATOM | 868 | N | ASN | A | 112 | −16.571 | −35.429 | −11.013 | 1.00 | 39.72 | A | N |
| ATOM | 869 | CA | ASN | A | 112 | −16.211 | −36.473 | −11.996 | 1.00 | 38.39 | A | C |
| ATOM | 870 | CB | ASN | A | 112 | −14.688 | −36.460 | −12.320 | 1.00 | 38.11 | A | C |
| ATOM | 871 | CG | ASN | A | 112 | −14.266 | −35.305 | −13.262 | 1.00 | 37.98 | A | C |
| ATOM | 872 | OD1 | ASN | A | 112 | −14.999 | −34.901 | −14.170 | 1.00 | 38.90 | A | O |
| ATOM | 873 | ND2 | ASN | A | 112 | −13.071 | −34.782 | −13.039 | 1.00 | 37.99 | A | N |
| ATOM | 874 | C | ASN | A | 112 | −16.660 | −37.890 | −11.576 | 1.00 | 37.82 | A | C |
| ATOM | 875 | O | ASN | A | 112 | −15.835 | −38.808 | −11.493 | 1.00 | 36.91 | A | O |
| ATOM | 876 | N | ASN | A | 113 | −17.957 | −38.074 | −11.336 | 1.00 | 37.02 | A | N |
| ATOM | 877 | CA | ASN | A | 113 | −18.427 | −39.382 | −10.923 | 1.00 | 37.51 | A | C |
| ATOM | 878 | CB | ASN | A | 113 | −17.979 | −39.699 | −9.489 | 1.00 | 38.57 | A | C |
| ATOM | 879 | CG | ASN | A | 113 | −17.709 | −41.176 | −9.295 | 1.00 | 43.30 | A | C |
| ATOM | 880 | OD1 | ASN | A | 113 | −16.666 | −41.694 | −9.683 | 1.00 | 44.68 | A | O |
| ATOM | 881 | ND2 | ASN | A | 113 | −18.682 | −41.875 | −8.730 | 1.00 | 50.44 | A | N |
| ATOM | 882 | C | ASN | A | 113 | −19.941 | −39.639 | −11.059 | 1.00 | 36.09 | A | C |
| ATOM | 883 | O | ASN | A | 113 | −20.717 | −38.706 | −11.308 | 1.00 | 35.79 | A | O |
| ATOM | 884 | N | ALA | A | 114 | −20.316 | −40.921 | −10.939 | 1.00 | 33.16 | A | N |
| ATOM | 885 | CA | ALA | A | 114 | −21.697 | −41.374 | −10.936 | 1.00 | 31.35 | A | C |
| ATOM | 886 | CB | ALA | A | 114 | −22.132 | −41.859 | −12.322 | 1.00 | 30.10 | A | C |
| ATOM | 887 | C | ALA | A | 114 | −21.681 | −42.536 | −9.981 | 1.00 | 30.28 | A | C |
| ATOM | 888 | O | ALA | A | 114 | −20.761 | −43.341 | −9.971 | 1.00 | 29.89 | A | O |
| ATOM | 889 | N | ALA | A | 115 | −22.685 | −42.665 | −9.152 | 1.00 | 28.73 | A | N |
| ATOM | 890 | CA | ALA | A | 115 | −22.587 | −43.775 | −8.219 | 1.00 | 27.85 | A | C |
| ATOM | 891 | CB | ALA | A | 115 | −21.783 | −43.333 | −6.923 | 1.00 | 26.42 | A | C |
| ATOM | 892 | C | ALA | A | 115 | −23.951 | −44.285 | −7.871 | 1.00 | 27.17 | A | C |
| ATOM | 893 | O | ALA | A | 115 | −24.932 | −43.551 | −7.903 | 1.00 | 26.95 | A | O |
| ATOM | 894 | N | TRP | A | 116 | −24.017 | −45.562 | −7.536 | 1.00 | 27.57 | A | N |
| ATOM | 895 | CA | TRP | A | 116 | −25.151 | −46.046 | −6.778 | 1.00 | 26.56 | A | C |
| ATOM | 896 | CB | TRP | A | 116 | −25.358 | −47.483 | −7.083 | 1.00 | 25.55 | A | C |
| ATOM | 897 | CG | TRP | A | 116 | −26.399 | −48.130 | −6.276 | 1.00 | 25.34 | A | C |
| ATOM | 898 | CD1 | TRP | A | 116 | −26.198 | −48.955 | −5.212 | 1.00 | 22.58 | A | C |
| ATOM | 899 | NE1 | TRP | A | 116 | −27.417 | −49.374 | −4.711 | 1.00 | 22.11 | A | N |
| ATOM | 900 | CE2 | TRP | A | 116 | −28.430 | −48.793 | −5.429 | 1.00 | 20.63 | A | C |
| ATOM | 901 | CD2 | TRP | A | 116 | −27.836 | −48.012 | −6.434 | 1.00 | 22.71 | A | C |
| ATOM | 902 | CE3 | TRP | A | 116 | −28.674 | −47.274 | −7.297 | 1.00 | 25.00 | A | C |
| ATOM | 903 | CZ3 | TRP | A | 116 | −30.096 | −47.399 | −7.160 | 1.00 | 20.26 | A | C |
| ATOM | 904 | CH2 | TRP | A | 116 | −30.633 | −48.228 | −6.183 | 1.00 | 20.58 | A | C |
| ATOM | 905 | CZ2 | TRP | A | 116 | −29.823 | −48.914 | −5.293 | 1.00 | 20.87 | A | C |
| ATOM | 906 | C | TRP | A | 116 | −24.826 | −45.858 | −5.300 | 1.00 | 28.31 | A | C |
| ATOM | 907 | O | TRP | A | 116 | −23.804 | −46.297 | −4.806 | 1.00 | 25.54 | A | O |
| ATOM | 908 | N | ILE | A | 117 | −25.719 | −45.166 | −4.600 | 1.00 | 31.41 | A | N |
| ATOM | 909 | CA | ILE | A | 117 | −25.501 | −44.758 | −3.224 | 1.00 | 33.35 | A | C |
| ATOM | 910 | CB | ILE | A | 117 | −25.708 | −43.237 | −3.204 | 1.00 | 34.35 | A | C |
| ATOM | 911 | CG1 | ILE | A | 117 | −24.445 | −42.583 | −2.753 | 1.00 | 35.74 | A | C |
| ATOM | 912 | CD1 | ILE | A | 117 | −23.596 | −42.437 | −3.940 | 1.00 | 36.48 | A | C |
| ATOM | 913 | CG2 | ILE | A | 117 | −27.052 | −42.729 | −2.567 | 1.00 | 32.98 | A | C |
| ATOM | 914 | C | ILE | A | 117 | −26.427 | −45.554 | −2.264 | 1.00 | 34.89 | A | C |
| ATOM | 915 | O | ILE | A | 117 | −26.928 | −45.013 | −1.236 | 1.00 | 36.20 | A | O |
| ATOM | 916 | N | GLY | A | 118 | −26.682 | −46.830 | −2.606 | 1.00 | 34.05 | A | N |
| ATOM | 917 | CA | GLY | A | 118 | −27.447 | −47.736 | −1.741 | 1.00 | 32.89 | A | C |
| ATOM | 918 | C | GLY | A | 118 | −28.958 | −47.716 | −2.031 | 1.00 | 33.33 | A | C |
| ATOM | 919 | O | GLY | A | 118 | −29.641 | −48.800 | −2.057 | 1.00 | 33.11 | A | O |
| ATOM | 920 | N | ASP | A | 119 | −29.494 | −46.514 | −2.243 | 1.00 | 30.79 | A | N |
| ATOM | 921 | CA | ASP | A | 119 | −30.881 | −46.411 | −2.581 | 1.00 | 31.30 | A | C |
| ATOM | 922 | CB | ASP | A | 119 | −31.688 | −45.834 | −1.399 | 1.00 | 33.35 | A | C |
| ATOM | 923 | CG | ASP | A | 119 | −31.091 | −44.540 | −0.838 | 1.00 | 37.71 | A | C |
| ATOM | 924 | OD1 | ASP | A | 119 | −31.838 | −43.790 | −0.087 | 1.00 | 41.58 | A | O |
| ATOM | 925 | OD2 | ASP | A | 119 | −29.858 | −44.313 | −1.105 | 1.00 | 38.82 | A | O |
| ATOM | 926 | C | ASP | A | 119 | −31.132 | −45.557 | −3.801 | 1.00 | 29.73 | A | C |
| ATOM | 927 | O | ASP | A | 119 | −32.270 | −45.400 | −4.168 | 1.00 | 29.65 | A | O |
| ATOM | 928 | N | GLN | A | 120 | −30.107 | −44.968 | −4.414 | 1.00 | 28.27 | A | N |
| ATOM | 929 | CA | GLN | A | 120 | −30.358 | −44.121 | −5.565 | 1.00 | 27.33 | A | C |
| ATOM | 930 | CB | GLN | A | 120 | −30.989 | −42.784 | −5.177 | 1.00 | 26.60 | A | C |
| ATOM | 931 | CG | GLN | A | 120 | −30.031 | −41.829 | −4.485 | 1.00 | 25.55 | A | C |
| ATOM | 932 | CD | GLN | A | 120 | −30.609 | −40.414 | −4.466 | 1.00 | 27.50 | A | C |
| ATOM | 933 | OE1 | GLN | A | 120 | −31.328 | −39.981 | −3.512 | 1.00 | 26.21 | A | O |
| ATOM | 934 | NE2 | GLN | A | 120 | −30.340 | −39.689 | −5.532 | 1.00 | 23.92 | A | N |
| ATOM | 935 | C | GLN | A | 120 | −29.117 | −43.901 | −6.390 | 1.00 | 28.01 | A | C |
| ATOM | 936 | O | GLN | A | 120 | −28.015 | −44.342 | −6.021 | 1.00 | 29.44 | A | O |
| ATOM | 937 | N | MET | A | 121 | −29.299 | −43.213 | −7.521 | 1.00 | 27.81 | A | N |
| ATOM | 938 | CA | MET | A | 121 | −28.212 | −42.851 | −8.400 | 1.00 | 26.90 | A | C |
| ATOM | 939 | CB | MET | A | 121 | −28.639 | −43.033 | −9.836 | 1.00 | 26.74 | A | C |
| ATOM | 940 | CG | MET | A | 121 | −28.830 | −44.451 | −10.264 | 1.00 | 28.73 | A | C |
| ATOM | 941 | SD | MET | A | 121 | −27.203 | −45.210 | −10.223 | 1.00 | 33.79 | A | S |
| ATOM | 942 | CE | MET | A | 121 | −26.289 | −44.011 | −11.268 | 1.00 | 35.84 | A | C |
| ATOM | 943 | C | MET | A | 121 | −27.925 | −41.400 | −8.183 | 1.00 | 26.76 | A | C |
| ATOM | 944 | O | MET | A | 121 | −28.878 | −40.606 | −8.009 | 1.00 | 27.02 | A | O |
| ATOM | 945 | N | ILE | A | 122 | −26.640 | −41.050 | −8.198 | 1.00 | 25.38 | A | N |
| ATOM | 946 | CA | ILE | A | 122 | −26.227 | −39.676 | −8.347 | 1.00 | 25.64 | A | C |
| ATOM | 947 | CB | ILE | A | 122 | −25.493 | −39.112 | −7.107 | 1.00 | 25.89 | A | C |
| ATOM | 948 | CG1 | ILE | A | 122 | −24.153 | −39.859 | −6.849 | 1.00 | 26.78 | A | C |
| ATOM | 949 | CD1 | ILE | A | 122 | −23.210 | −39.229 | −5.778 | 1.00 | 28.66 | A | C |
| ATOM | 950 | CG2 | ILE | A | 122 | −26.401 | −39.291 | −5.890 | 1.00 | 25.18 | A | C |
| ATOM | 951 | C | ILE | A | 122 | −25.352 | −39.591 | −9.585 | 1.00 | 25.99 | A | C |
| ATOM | 952 | O | ILE | A | 122 | −24.582 | −40.512 | −9.892 | 1.00 | 24.65 | A | O |
| ATOM | 953 | N | TYR | A | 123 | −25.477 | −38.468 | −10.292 | 1.00 | 26.83 | A | N |
| ATOM | 954 | CA | TYR | A | 123 | −24.684 | −38.214 | −11.479 | 1.00 | 27.50 | A | C |
| ATOM | 955 | CB | TYR | A | 123 | −25.620 | −38.158 | −12.642 | 1.00 | 26.01 | A | C |
| ATOM | 956 | CG | TYR | A | 123 | −26.202 | −39.467 | −12.987 | 1.00 | 28.56 | A | C |
| ATOM | 957 | CD1 | TYR | A | 123 | −25.458 | −40.424 | −13.696 | 1.00 | 26.12 | A | C |
| ATOM | 958 | CE1 | TYR | A | 123 | −26.016 | −41.621 | −14.047 | 1.00 | 27.49 | A | C |
| ATOM | 959 | CZ | TYR | A | 123 | −27.340 | −41.890 | −13.696 | 1.00 | 29.40 | A | C |
| ATOM | 960 | OH | TYR | A | 123 | −27.940 | −43.075 | −14.048 | 1.00 | 29.36 | A | O |
| ATOM | 961 | CE2 | TYR | A | 123 | −28.089 | −40.965 | −12.974 | 1.00 | 29.36 | A | C |
| ATOM | 962 | CD2 | TYR | A | 123 | −27.525 | −39.761 | −12.637 | 1.00 | 28.86 | A | C |
| ATOM | 963 | C | TYR | A | 123 | −24.000 | −36.866 | −11.410 | 1.00 | 27.64 | A | C |
| ATOM | 964 | O | TYR | A | 123 | −24.682 | −35.864 | −11.416 | 1.00 | 27.65 | A | O |
| ATOM | 965 | N | GLY | A | 124 | −22.675 | −36.814 | −11.397 | 1.00 | 28.67 | A | N |
| ATOM | 966 | CA | GLY | A | 124 | −21.985 | −35.522 | −11.636 | 1.00 | 29.95 | A | C |
| ATOM | 967 | C | GLY | A | 124 | −22.156 | −34.939 | −13.056 | 1.00 | 32.16 | A | C |
| ATOM | 968 | O | GLY | A | 124 | −22.633 | −35.631 | −14.029 | 1.00 | 31.54 | A | O |
| ATOM | 969 | N | ASP | A | 125 | −21.774 | −33.667 | −13.197 | 1.00 | 32.99 | A | N |
| ATOM | 970 | CA | ASP | A | 125 | −21.679 | −33.034 | −14.514 | 1.00 | 34.33 | A | C |
| ATOM | 971 | CB | ASP | A | 125 | −21.971 | −31.562 | −14.386 | 1.00 | 34.40 | A | C |
| ATOM | 972 | CG | ASP | A | 125 | −23.380 | −31.287 | −13.965 | 1.00 | 37.99 | A | C |
| ATOM | 973 | OD1 | ASP | A | 125 | −24.336 | −31.852 | −14.549 | 1.00 | 38.07 | A | O |
| ATOM | 974 | OD2 | ASP | A | 125 | −23.520 | −30.455 | −13.045 | 1.00 | 44.23 | A | O |
| ATOM | 975 | C | ASP | A | 125 | −20.288 | −33.194 | −15.165 | 1.00 | 34.98 | A | C |
| ATOM | 976 | O | ASP | A | 125 | −20.102 | −32.863 | −16.340 | 1.00 | 34.73 | A | O |
| ATOM | 977 | N | GLY | A | 126 | −19.312 | −33.685 | −14.397 | 1.00 | 35.47 | A | N |
| ATOM | 978 | CA | GLY | A | 126 | −17.931 | −33.785 | −14.893 | 1.00 | 36.66 | A | C |
| ATOM | 979 | C | GLY | A | 126 | −17.350 | −32.381 | −15.012 | 1.00 | 37.88 | A | C |
| ATOM | 980 | O | GLY | A | 126 | −18.083 | −31.394 | −14.794 | 1.00 | 38.63 | A | O |
| ATOM | 981 | N | ASP | A | 127 | −16.060 | −32.284 | −15.376 | 1.00 | 37.62 | A | N |
| ATOM | 982 | CA | ASP | A | 127 | −15.330 | −31.004 | −15.363 | 1.00 | 37.72 | A | C |
| ATOM | 983 | CB | ASP | A | 127 | −13.996 | −31.138 | −14.637 | 1.00 | 37.18 | A | C |
| ATOM | 984 | CG | ASP | A | 127 | −13.106 | −32.173 | −15.272 | 1.00 | 37.42 | A | C |
| ATOM | 985 | OD1 | ASP | A | 127 | −13.431 | −32.597 | −16.405 | 1.00 | 36.84 | A | O |
| ATOM | 986 | OD2 | ASP | A | 127 | −12.107 | −32.578 | −14.631 | 1.00 | 35.38 | A | O |
| ATOM | 987 | C | ASP | A | 127 | −15.057 | −30.433 | −16.756 | 1.00 | 37.76 | A | C |
| ATOM | 988 | O | ASP | A | 127 | −14.229 | −29.510 | −16.877 | 1.00 | 37.23 | A | O |
| ATOM | 989 | N | GLY | A | 128 | −15.750 | −30.980 | −17.767 | 1.00 | 37.19 | A | N |
| ATOM | 990 | CA | GLY | A | 128 | −15.485 | −30.716 | −19.179 | 1.00 | 37.16 | A | C |
| ATOM | 991 | C | GLY | A | 128 | −14.082 | −31.007 | −19.715 | 1.00 | 38.07 | A | C |
| ATOM | 992 | O | GLY | A | 128 | −13.859 | −30.746 | −20.890 | 1.00 | 39.17 | A | O |
| ATOM | 993 | N | ILE | A | 129 | −13.139 | −31.495 | −18.880 | 1.00 | 37.52 | A | N |
| ATOM | 994 | CA | ILE | A | 129 | −11.811 | −31.964 | −19.309 | 1.00 | 36.84 | A | C |
| ATOM | 995 | CB | ILE | A | 129 | −10.651 | −31.606 | −18.310 | 1.00 | 37.50 | A | C |
| ATOM | 996 | CG1 | ILE | A | 129 | −10.767 | −30.184 | −17.642 | 1.00 | 38.34 | A | C |
| ATOM | 997 | CD1 | ILE | A | 129 | −10.877 | −28.983 | −18.606 | 1.00 | 42.13 | A | C |
| ATOM | 998 | CG2 | ILE | A | 129 | −9.254 | −31.938 | −18.942 | 1.00 | 33.49 | A | C |
| ATOM | 999 | C | ILE | A | 129 | −11.819 | −33.512 | −19.349 | 1.00 | 37.57 | A | C |
| ATOM | 1000 | O | ILE | A | 129 | −11.514 | −34.142 | −20.378 | 1.00 | 38.01 | A | O |
| ATOM | 1001 | N | LEU | A | 130 | −12.150 | −34.132 | −18.216 | 1.00 | 36.97 | A | N |
| ATOM | 1002 | CA | LEU | A | 130 | −12.229 | −35.595 | −18.099 | 1.00 | 36.57 | A | C |
| ATOM | 1003 | CB | LEU | A | 130 | −11.936 | −35.959 | −16.669 | 1.00 | 37.18 | A | C |
| ATOM | 1004 | CG | LEU | A | 130 | −10.659 | −36.632 | −16.212 | 1.00 | 43.85 | A | C |
| ATOM | 1005 | CD1 | LEU | A | 130 | −10.785 | −38.205 | −16.580 | 1.00 | 46.49 | A | C |
| ATOM | 1006 | CD2 | LEU | A | 130 | −9.269 | −35.957 | −16.695 | 1.00 | 47.25 | A | C |
| ATOM | 1007 | C | LEU | A | 130 | −13.599 | −36.199 | −18.584 | 1.00 | 35.25 | A | C |
| ATOM | 1008 | O | LEU | A | 130 | −13.644 | −37.081 | −19.447 | 1.00 | 33.76 | A | O |
| ATOM | 1009 | N | PHE | A | 131 | −14.713 | −35.687 | −18.074 | 1.00 | 34.61 | A | N |
| ATOM | 1010 | CA | PHE | A | 131 | −16.039 | −36.253 | −18.388 | 1.00 | 34.15 | A | C |
| ATOM | 1011 | CB | PHE | A | 131 | −16.657 | −36.934 | −17.161 | 1.00 | 34.95 | A | C |
| ATOM | 1012 | CG | PHE | A | 131 | −15.891 | −38.104 | −16.672 | 1.00 | 38.14 | A | C |
| ATOM | 1013 | CD1 | PHE | A | 131 | −15.883 | −39.308 | −17.404 | 1.00 | 41.58 | A | C |
| ATOM | 1014 | CE1 | PHE | A | 131 | −15.151 | −40.406 | −16.966 | 1.00 | 44.73 | A | C |
| ATOM | 1015 | CZ | PHE | A | 131 | −14.387 | −40.309 | −15.767 | 1.00 | 48.86 | A | C |
| ATOM | 1016 | CE2 | PHE | A | 131 | −14.398 | −39.106 | −15.023 | 1.00 | 47.18 | A | C |
| ATOM | 1017 | CD2 | PHE | A | 131 | −15.162 | −38.011 | −15.501 | 1.00 | 41.75 | A | C |
| ATOM | 1018 | C | PHE | A | 131 | −17.011 | −35.199 | −18.809 | 1.00 | 32.89 | A | C |
| ATOM | 1019 | O | PHE | A | 131 | −16.964 | −34.096 | −18.280 | 1.00 | 33.01 | A | O |
| ATOM | 1020 | N | SER | A | 132 | −17.907 | −35.526 | −19.737 | 1.00 | 30.84 | A | N |
| ATOM | 1021 | CA | SER | A | 132 | −19.070 | −34.702 | −19.916 | 1.00 | 29.80 | A | C |
| ATOM | 1022 | CB | SER | A | 132 | −19.624 | −34.804 | −21.323 | 1.00 | 29.21 | A | C |
| ATOM | 1023 | OG | SER | A | 132 | −19.674 | −36.134 | −21.697 | 1.00 | 30.42 | A | O |
| ATOM | 1024 | C | SER | A | 132 | −20.048 | −35.190 | −18.859 | 1.00 | 29.53 | A | C |
| ATOM | 1025 | O | SER | A | 132 | −19.685 | −36.006 | −18.036 | 1.00 | 30.08 | A | O |
| ATOM | 1026 | N | PRO | A | 133 | −21.272 | −34.647 | −18.817 | 1.00 | 29.36 | A | N |
| ATOM | 1027 | CA | PRO | A | 133 | −22.190 | −35.114 | −17.725 | 1.00 | 27.74 | A | C |
| ATOM | 1028 | CB | PRO | A | 133 | −23.429 | −34.274 | −17.954 | 1.00 | 27.87 | A | C |
| ATOM | 1029 | CG | PRO | A | 133 | −22.824 | −32.907 | −18.594 | 1.00 | 28.87 | A | C |
| ATOM | 1030 | CD | PRO | A | 133 | −21.681 | −33.354 | −19.443 | 1.00 | 28.76 | A | C |
| ATOM | 1031 | C | PRO | A | 133 | −22.473 | −36.640 | −17.708 | 1.00 | 27.79 | A | C |
| ATOM | 1032 | O | PRO | A | 133 | −22.816 | −37.261 | −18.724 | 1.00 | 28.94 | A | O |
| ATOM | 1033 | N | LEU | A | 134 | −22.333 | −37.266 | −16.551 | 1.00 | 27.55 | A | N |
| ATOM | 1034 | CA | LEU | A | 134 | −22.239 | −38.723 | −16.535 | 1.00 | 26.30 | A | C |
| ATOM | 1035 | CB | LEU | A | 134 | −21.559 | −39.226 | −15.287 | 1.00 | 26.50 | A | C |
| ATOM | 1036 | CG | LEU | A | 134 | −20.106 | −38.765 | −15.416 | 1.00 | 26.04 | A | C |
| ATOM | 1037 | CD1 | LEU | A | 134 | −19.838 | −37.590 | −14.496 | 1.00 | 24.48 | A | C |
| ATOM | 1038 | CD2 | LEU | A | 134 | −19.191 | −39.925 | −15.156 | 1.00 | 25.59 | A | C |
| ATOM | 1039 | C | LEU | A | 134 | −23.503 | −39.514 | −16.841 | 1.00 | 26.32 | A | C |
| ATOM | 1040 | O | LEU | A | 134 | −23.407 | −40.717 | −17.164 | 1.00 | 26.47 | A | O |
| ATOM | 1041 | N | SER | A | 135 | −24.671 | −38.862 | −16.804 | 1.00 | 24.53 | A | N |
| ATOM | 1042 | CA | SER | A | 135 | −25.889 | −39.545 | −17.217 | 1.00 | 23.28 | A | C |
| ATOM | 1043 | CB | SER | A | 135 | −27.116 | −38.743 | −16.855 | 1.00 | 23.25 | A | C |
| ATOM | 1044 | OG | SER | A | 135 | −26.917 | −37.361 | −17.117 | 1.00 | 25.21 | A | O |
| ATOM | 1045 | C | SER | A | 135 | −25.899 | −39.891 | −18.701 | 1.00 | 22.93 | A | C |
| ATOM | 1046 | O | SER | A | 135 | −26.695 | −40.741 | −19.145 | 1.00 | 21.32 | A | O |
| ATOM | 1047 | N | GLY | A | 136 | −24.986 | −39.272 | −19.467 | 1.00 | 23.76 | A | N |
| ATOM | 1048 | CA | GLY | A | 136 | −25.006 | −39.438 | −20.917 | 1.00 | 24.14 | A | C |
| ATOM | 1049 | C | GLY | A | 136 | −24.621 | −40.823 | −21.457 | 1.00 | 24.78 | A | C |
| ATOM | 1050 | O | GLY | A | 136 | −24.722 | −41.048 | −22.656 | 1.00 | 26.30 | A | O |
| ATOM | 1051 | N | SER | A | 137 | −24.240 | −41.758 | −20.584 | 1.00 | 24.70 | A | N |
| ATOM | 1052 | CA | SER | A | 137 | −23.777 | −43.079 | −20.985 | 1.00 | 24.83 | A | C |
| ATOM | 1053 | CB | SER | A | 137 | −22.367 | −43.311 | −20.477 | 1.00 | 24.69 | A | C |
| ATOM | 1054 | OG | SER | A | 137 | −22.103 | −44.685 | −20.366 | 1.00 | 25.08 | A | O |
| ATOM | 1055 | C | SER | A | 137 | −24.685 | −44.169 | −20.437 | 1.00 | 25.13 | A | C |
| ATOM | 1056 | O | SER | A | 137 | −24.759 | −44.368 | −19.222 | 1.00 | 24.93 | A | O |
| ATOM | 1057 | N | LEU | A | 138 | −25.412 | −44.849 | −21.328 | 1.00 | 24.00 | A | N |
| ATOM | 1058 | CA | LEU | A | 138 | −26.252 | −45.962 | −20.897 | 1.00 | 22.97 | A | C |
| ATOM | 1059 | CB | LEU | A | 138 | −27.032 | −46.501 | −22.064 | 1.00 | 21.65 | A | C |
| ATOM | 1060 | CG | LEU | A | 138 | −27.858 | −47.765 | −21.927 | 1.00 | 22.78 | A | C |
| ATOM | 1061 | CD1 | LEU | A | 138 | −28.727 | −47.666 | −20.674 | 1.00 | 21.27 | A | C |
| ATOM | 1062 | CD2 | LEU | A | 138 | −28.726 | −47.916 | −23.246 | 1.00 | 18.55 | A | C |
| ATOM | 1063 | C | LEU | A | 138 | −25.439 | −47.092 | −20.237 | 1.00 | 22.85 | A | C |
| ATOM | 1064 | O | LEU | A | 138 | −25.874 | −47.701 | −19.254 | 1.00 | 23.50 | A | O |
| ATOM | 1065 | N | ASP | A | 139 | −24.260 | −47.382 | −20.755 | 1.00 | 22.10 | A | N |
| ATOM | 1066 | CA | ASP | A | 139 | −23.512 | −48.490 | −20.171 | 1.00 | 21.45 | A | C |
| ATOM | 1067 | CB | ASP | A | 139 | −22.459 | −49.121 | −21.135 | 1.00 | 21.46 | A | C |
| ATOM | 1068 | CG | ASP | A | 139 | −21.378 | −48.184 | −21.548 | 1.00 | 20.87 | A | C |
| ATOM | 1069 | OD1 | ASP | A | 139 | −20.693 | −47.613 | −20.695 | 1.00 | 19.56 | A | O |
| ATOM | 1070 | OD2 | ASP | A | 139 | −21.230 | −48.006 | −22.768 | 1.00 | 26.03 | A | O |
| ATOM | 1071 | C | ASP | A | 139 | −22.996 | −48.153 | −18.787 | 1.00 | 20.85 | A | C |
| ATOM | 1072 | O | ASP | A | 139 | −22.941 | −49.032 | −17.951 | 1.00 | 20.86 | A | O |
| ATOM | 1073 | N | VAL | A | 140 | −22.708 | −46.881 | −18.518 | 1.00 | 20.51 | A | N |
| ATOM | 1074 | CA | VAL | A | 140 | −22.297 | −46.459 | −17.184 | 1.00 | 20.92 | A | C |
| ATOM | 1075 | CB | VAL | A | 140 | −21.593 | −45.074 | −17.222 | 1.00 | 21.74 | A | C |
| ATOM | 1076 | CG1 | VAL | A | 140 | −21.631 | −44.384 | −15.832 | 1.00 | 21.41 | A | C |
| ATOM | 1077 | CG2 | VAL | A | 140 | −20.164 | −45.249 | −17.728 | 1.00 | 21.41 | A | C |
| ATOM | 1078 | C | VAL | A | 140 | −23.476 | −46.498 | −16.156 | 1.00 | 22.07 | A | C |
| ATOM | 1079 | O | VAL | A | 140 | −23.291 | −46.870 | −14.941 | 1.00 | 21.54 | A | O |
| ATOM | 1080 | N | THR | A | 141 | −24.676 | −46.178 | −16.641 | 1.00 | 20.44 | A | N |
| ATOM | 1081 | CA | THR | A | 141 | −25.833 | −46.224 | −15.813 | 1.00 | 20.85 | A | C |
| ATOM | 1082 | CB | THR | A | 141 | −26.984 | −45.523 | −16.496 | 1.00 | 21.74 | A | C |
| ATOM | 1083 | OG1 | THR | A | 141 | −26.849 | −44.113 | −16.306 | 1.00 | 23.68 | A | O |
| ATOM | 1084 | CG2 | THR | A | 141 | −28.304 | −45.978 | −15.950 | 1.00 | 23.15 | A | C |
| ATOM | 1085 | C | THR | A | 141 | −26.163 | −47.695 | −15.559 | 1.00 | 21.53 | A | C |
| ATOM | 1086 | O | THR | A | 141 | −26.583 | −48.086 | −14.444 | 1.00 | 21.87 | A | O |
| ATOM | 1087 | N | ALA | A | 142 | −25.964 | −48.547 | −16.552 | 1.00 | 20.30 | A | N |
| ATOM | 1088 | CA | ALA | A | 142 | −26.249 | −49.949 | −16.264 | 1.00 | 20.29 | A | C |
| ATOM | 1089 | CB | ALA | A | 142 | −26.338 | −50.784 | −17.552 | 1.00 | 18.91 | A | C |
| ATOM | 1090 | C | ALA | A | 142 | −25.177 | −50.503 | −15.260 | 1.00 | 20.38 | A | C |
| ATOM | 1091 | O | ALA | A | 142 | −25.511 | −51.271 | −14.311 | 1.00 | 19.43 | A | O |
| ATOM | 1092 | N | HIS | A | 143 | −23.912 | −50.094 | −15.455 | 1.00 | 19.82 | A | N |
| ATOM | 1093 | CA | HIS | A | 143 | −22.832 | −50.454 | −14.479 | 1.00 | 20.46 | A | C |
| ATOM | 1094 | CB | HIS | A | 143 | −21.554 | −49.725 | −14.830 | 1.00 | 19.52 | A | C |
| ATOM | 1095 | CG | HIS | A | 143 | −20.477 | −49.854 | −13.814 | 1.00 | 20.81 | A | C |
| ATOM | 1096 | ND1 | HIS | A | 143 | −19.292 | −50.509 | −14.068 | 1.00 | 20.20 | A | N |
| ATOM | 1097 | CE1 | HIS | A | 143 | −18.521 | −50.442 | −12.994 | 1.00 | 21.05 | A | C |
| ATOM | 1098 | NE2 | HIS | A | 143 | −19.171 | −49.786 | −12.044 | 1.00 | 20.22 | A | N |
| ATOM | 1099 | CD2 | HIS | A | 143 | −20.378 | −49.370 | −12.548 | 1.00 | 23.25 | A | C |
| ATOM | 1100 | C | HIS | A | 143 | −23.266 | −50.149 | −13.024 | 1.00 | 20.74 | A | C |
| ATOM | 1101 | O | HIS | A | 143 | −23.219 | −50.990 | −12.137 | 1.00 | 20.11 | A | O |
| ATOM | 1102 | N | GLU | A | 144 | −23.722 | −48.929 | −12.824 | 1.00 | 22.35 | A | N |
| ATOM | 1103 | CA | GLU | A | 144 | −24.156 | −48.452 | −11.507 | 1.00 | 24.02 | A | C |
| ATOM | 1104 | CB | GLU | A | 144 | −24.237 | −46.884 | −11.486 | 1.00 | 23.44 | A | C |
| ATOM | 1105 | CG | GLU | A | 144 | −22.935 | −46.184 | −11.827 | 1.00 | 23.80 | A | C |
| ATOM | 1106 | CD | GLU | A | 144 | −21.806 | −46.396 | −10.835 | 1.00 | 31.05 | A | C |
| ATOM | 1107 | OE1 | GLU | A | 144 | −20.600 | −46.173 | −11.189 | 1.00 | 37.21 | A | O |
| ATOM | 1108 | OE2 | GLU | A | 144 | −22.093 | −46.768 | −9.676 | 1.00 | 33.01 | A | O |
| ATOM | 1109 | C | GLU | A | 144 | −25.452 | −49.143 | −11.015 | 1.00 | 23.04 | A | C |
| ATOM | 1110 | O | GLU | A | 144 | −25.530 | −49.577 | −9.873 | 1.00 | 24.42 | A | O |
| ATOM | 1111 | N | MET | A | 145 | −26.441 | −49.319 | −11.863 | 1.00 | 22.79 | A | N |
| ATOM | 1112 | CA | MET | A | 145 | −27.614 | −50.030 | −11.390 | 1.00 | 23.27 | A | C |
| ATOM | 1113 | CB | MET | A | 145 | −28.698 | −50.127 | −12.442 | 1.00 | 23.04 | A | C |
| ATOM | 1114 | CG | MET | A | 145 | −29.845 | −49.154 | −12.293 | 1.00 | 30.13 | A | C |
| ATOM | 1115 | SD | MET | A | 145 | −30.091 | −48.329 | −10.642 | 1.00 | 38.83 | A | S |
| ATOM | 1116 | CE | MET | A | 145 | −28.992 | −46.995 | −10.984 | 1.00 | 30.09 | A | C |
| ATOM | 1117 | C | MET | A | 145 | −27.255 | −51.425 | −10.899 | 1.00 | 23.43 | A | C |
| ATOM | 1118 | O | MET | A | 145 | −27.820 | −51.930 | −9.890 | 1.00 | 23.83 | A | O |
| ATOM | 1119 | N | THR | A | 146 | −26.308 | −52.042 | −11.602 | 1.00 | 22.59 | A | N |
| ATOM | 1120 | CA | THR | A | 146 | −25.933 | −53.408 | −11.333 | 1.00 | 22.67 | A | C |
| ATOM | 1121 | CB | THR | A | 146 | −25.012 | −53.943 | −12.471 | 1.00 | 23.17 | A | C |
| ATOM | 1122 | OG1 | THR | A | 146 | −25.757 | −53.956 | −13.691 | 1.00 | 21.92 | A | O |
| ATOM | 1123 | CG2 | THR | A | 146 | −24.520 | −55.370 | −12.182 | 1.00 | 22.76 | A | C |
| ATOM | 1124 | C | THR | A | 146 | −25.310 | −53.564 | −9.938 | 1.00 | 23.21 | A | C |
| ATOM | 1125 | O | THR | A | 146 | −25.425 | −54.655 | −9.340 | 1.00 | 23.05 | A | O |
| ATOM | 1126 | N | HIS | A | 147 | −24.658 | −52.502 | −9.417 | 1.00 | 22.84 | A | N |
| ATOM | 1127 | CA | HIS | A | 147 | −24.120 | −52.535 | −8.034 | 1.00 | 23.53 | A | C |
| ATOM | 1128 | CB | HIS | A | 147 | −23.409 | −51.247 | −7.640 | 1.00 | 23.43 | A | C |
| ATOM | 1129 | CG | HIS | A | 147 | −22.024 | −51.094 | −8.195 | 1.00 | 25.63 | A | C |
| ATOM | 1130 | ND1 | HIS | A | 147 | −21.056 | −52.081 | −8.097 | 1.00 | 22.45 | A | N |
| ATOM | 1131 | CE1 | HIS | A | 147 | −19.935 | −51.638 | −8.662 | 1.00 | 23.31 | A | C |
| ATOM | 1132 | NE2 | HIS | A | 147 | −20.139 | −50.412 | −9.125 | 1.00 | 22.19 | A | N |
| ATOM | 1133 | CD2 | HIS | A | 147 | −21.432 | −50.042 | −8.838 | 1.00 | 24.54 | A | C |
| ATOM | 1134 | C | HIS | A | 147 | −25.274 | −52.736 | −7.045 | 1.00 | 23.74 | A | C |
| ATOM | 1135 | O | HIS | A | 147 | −25.099 | −53.368 | −6.037 | 1.00 | 22.83 | A | O |
| ATOM | 1136 | N | GLY | A | 148 | −26.454 | −52.192 | −7.379 | 1.00 | 24.04 | A | N |
| ATOM | 1137 | CA | GLY | A | 148 | −27.645 | −52.306 | −6.567 | 1.00 | 23.86 | A | C |
| ATOM | 1138 | C | GLY | A | 148 | −28.157 | −53.715 | −6.634 | 1.00 | 24.32 | A | C |
| ATOM | 1139 | O | GLY | A | 148 | −28.523 | −54.294 | −5.597 | 1.00 | 26.05 | A | O |
| ATOM | 1140 | N | VAL | A | 149 | −28.141 | −54.301 | −7.825 | 1.00 | 23.92 | A | N |
| ATOM | 1141 | CA | VAL | A | 149 | −28.526 | −55.705 | −7.988 | 1.00 | 22.96 | A | C |
| ATOM | 1142 | CB | VAL | A | 149 | −28.493 | −56.127 | −9.460 | 1.00 | 22.83 | A | C |
| ATOM | 1143 | CG1 | VAL | A | 149 | −28.656 | −57.615 | −9.564 | 1.00 | 18.85 | A | C |
| ATOM | 1144 | CG2 | VAL | A | 149 | −29.512 | −55.358 | −10.259 | 1.00 | 20.25 | A | C |
| ATOM | 1145 | C | VAL | A | 149 | −27.569 | −56.616 | −7.200 | 1.00 | 24.15 | A | C |
| ATOM | 1146 | O | VAL | A | 149 | −27.990 | −57.541 | −6.453 | 1.00 | 25.47 | A | O |
| ATOM | 1147 | N | THR | A | 150 | −26.278 | −56.343 | −7.332 | 1.00 | 23.75 | A | N |
| ATOM | 1148 | CA | THR | A | 150 | −25.284 | −57.085 | −6.559 | 1.00 | 24.24 | A | C |
| ATOM | 1149 | CB | THR | A | 150 | −23.863 | −56.666 | −6.946 | 1.00 | 23.99 | A | C |
| ATOM | 1150 | OG1 | THR | A | 150 | −23.602 | −57.125 | −8.283 | 1.00 | 22.23 | A | O |
| ATOM | 1151 | CG2 | THR | A | 150 | −22.845 | −57.248 | −6.005 | 1.00 | 20.09 | A | C |
| ATOM | 1152 | C | THR | A | 150 | −25.514 | −56.948 | −5.041 | 1.00 | 25.81 | A | C |
| ATOM | 1153 | O | THR | A | 150 | −25.448 | −57.939 | −4.305 | 1.00 | 26.18 | A | O |
| ATOM | 1154 | N | GLN | A | 151 | −25.802 | −55.731 | −4.594 | 1.00 | 26.07 | A | N |
| ATOM | 1155 | CA | GLN | A | 151 | −25.946 | −55.438 | −3.194 | 1.00 | 27.46 | A | C |
| ATOM | 1156 | CB | GLN | A | 151 | −26.045 | −53.951 | −3.078 | 1.00 | 28.06 | A | C |
| ATOM | 1157 | CG | GLN | A | 151 | −26.508 | −53.295 | −1.761 | 1.00 | 34.50 | A | C |
| ATOM | 1158 | CD | GLN | A | 151 | −26.880 | −51.784 | −2.065 | 1.00 | 38.36 | A | C |
| ATOM | 1159 | OE1 | GLN | A | 151 | −28.045 | −51.379 | −1.940 | 1.00 | 37.38 | A | O |
| ATOM | 1160 | NE2 | GLN | A | 151 | −25.886 | −51.007 | −2.557 | 1.00 | 37.31 | A | N |
| ATOM | 1161 | C | GLN | A | 151 | −27.146 | −56.201 | −2.585 | 1.00 | 27.11 | A | C |
| ATOM | 1162 | O | GLN | A | 151 | −27.025 | −56.777 | −1.499 | 1.00 | 26.88 | A | O |
| ATOM | 1163 | N | GLU | A | 152 | −28.218 | −56.308 | −3.358 | 1.00 | 25.84 | A | N |
| ATOM | 1164 | CA | GLU | A | 152 | −29.411 | −57.045 | −2.979 | 1.00 | 26.00 | A | C |
| ATOM | 1165 | CB | GLU | A | 152 | −30.601 | −56.525 | −3.811 | 1.00 | 24.38 | A | C |
| ATOM | 1166 | CG | GLU | A | 152 | −30.962 | −55.115 | −3.474 | 1.00 | 28.24 | A | C |
| ATOM | 1167 | CD | GLU | A | 152 | −31.418 | −55.027 | −2.050 | 1.00 | 30.02 | A | C |
| ATOM | 1168 | OE1 | GLU | A | 152 | −32.452 | −55.626 | −1.757 | 1.00 | 33.64 | A | O |
| ATOM | 1169 | OE2 | GLU | A | 152 | −30.694 | −54.464 | −1.202 | 1.00 | 32.95 | A | O |
| ATOM | 1170 | C | GLU | A | 152 | −29.337 | −58.566 | −3.183 | 1.00 | 25.65 | A | C |
| ATOM | 1171 | O | GLU | A | 152 | −30.318 | −59.303 | −2.924 | 1.00 | 25.43 | A | O |
| ATOM | 1172 | N | THR | A | 153 | −28.193 | −59.026 | −3.670 | 1.00 | 24.78 | A | N |
| ATOM | 1173 | CA | THR | A | 153 | −28.036 | −60.395 | −4.148 | 1.00 | 23.39 | A | C |
| ATOM | 1174 | CB | THR | A | 153 | −28.026 | −60.258 | −5.660 | 1.00 | 24.08 | A | C |
| ATOM | 1175 | OG1 | THR | A | 153 | −29.320 | −60.620 | −6.229 | 1.00 | 27.23 | A | O |
| ATOM | 1176 | CG2 | THR | A | 153 | −27.026 | −60.967 | −6.210 | 1.00 | 21.52 | A | C |
| ATOM | 1177 | C | THR | A | 153 | −26.756 | −60.999 | −3.519 | 1.00 | 23.00 | A | C |
| ATOM | 1178 | O | THR | A | 153 | −26.707 | −61.211 | −2.325 | 1.00 | 22.29 | A | O |
| ATOM | 1179 | N | ALA | A | 154 | −25.685 | −61.182 | −4.301 | 1.00 | 22.49 | A | N |
| ATOM | 1180 | CA | ALA | A | 154 | −24.415 | −61.685 | −3.835 | 1.00 | 20.07 | A | C |
| ATOM | 1181 | CB | ALA | A | 154 | −23.506 | −61.833 | −4.985 | 1.00 | 19.57 | A | C |
| ATOM | 1182 | C | ALA | A | 154 | −23.744 | −60.845 | −2.758 | 1.00 | 20.31 | A | C |
| ATOM | 1183 | O | ALA | A | 154 | −22.986 | −61.382 | −1.911 | 1.00 | 19.99 | A | O |
| ATOM | 1184 | N | ASN | A | 155 | −23.935 | −59.540 | −2.840 | 1.00 | 19.80 | A | N |
| ATOM | 1185 | CA | ASN | A | 155 | −23.290 | −58.608 | −1.924 | 1.00 | 20.54 | A | C |
| ATOM | 1186 | CB | ASN | A | 155 | −23.959 | −58.712 | −0.525 | 1.00 | 20.57 | A | C |
| ATOM | 1187 | CG | ASN | A | 155 | −23.827 | −57.429 | 0.321 | 1.00 | 21.69 | A | C |
| ATOM | 1188 | OD1 | ASN | A | 155 | −24.120 | −57.444 | 1.545 | 1.00 | 24.14 | A | O |
| ATOM | 1189 | ND2 | ASN | A | 155 | −23.340 | −56.352 | −0.283 | 1.00 | 18.66 | A | N |
| ATOM | 1190 | C | ASN | A | 155 | −21.753 | −58.751 | −1.853 | 1.00 | 20.95 | A | C |
| ATOM | 1191 | O | ASN | A | 155 | −21.134 | −58.840 | −0.755 | 1.00 | 21.27 | A | O |
| ATOM | 1192 | N | LEU | A | 156 | −21.109 | −58.792 | −3.009 | 1.00 | 21.01 | A | N |
| ATOM | 1193 | CA | LEU | A | 156 | −19.650 | −58.923 | −3.002 | 1.00 | 20.82 | A | C |
| ATOM | 1194 | CB | LEU | A | 156 | −19.137 | −58.933 | −4.433 | 1.00 | 20.60 | A | C |
| ATOM | 1195 | CG | LEU | A | 156 | −19.621 | −60.098 | −5.269 | 1.00 | 19.99 | A | C |
| ATOM | 1196 | CD1 | LEU | A | 156 | −19.739 | −59.645 | −6.739 | 1.00 | 18.05 | A | C |
| ATOM | 1197 | CD2 | LEU | A | 156 | −18.615 | −61.191 | −5.062 | 1.00 | 15.60 | A | C |
| ATOM | 1198 | C | LEU | A | 156 | −18.998 | −57.809 | −2.193 | 1.00 | 20.75 | A | C |
| ATOM | 1199 | O | LEU | A | 156 | −19.303 | −56.638 | −2.377 | 1.00 | 21.10 | A | O |
| ATOM | 1200 | N | ASN | A | 157 | −18.135 | −58.188 | −1.268 | 1.00 | 21.00 | A | N |
| ATOM | 1201 | CA | ASN | A | 157 | −17.314 | −57.263 | −0.529 | 1.00 | 21.48 | A | C |
| ATOM | 1202 | CB | ASN | A | 157 | −16.367 | −58.077 | 0.293 | 1.00 | 20.96 | A | C |
| ATOM | 1203 | CG | ASN | A | 157 | −17.026 | −58.748 | 1.430 | 1.00 | 19.87 | A | C |
| ATOM | 1204 | OD1 | ASN | A | 157 | −18.154 | −58.388 | 1.838 | 1.00 | 24.09 | A | O |
| ATOM | 1205 | ND2 | ASN | A | 157 | −16.337 | −59.738 | 1.977 | 1.00 | 14.00 | A | N |
| ATOM | 1206 | C | ASN | A | 157 | −16.458 | −56.356 | −1.436 | 1.00 | 24.39 | A | C |
| ATOM | 1207 | O | ASN | A | 157 | −15.973 | −56.793 | −2.498 | 1.00 | 24.92 | A | O |
| ATOM | 1208 | N | TYR | A | 158 | −16.273 | −55.110 | −1.031 | 1.00 | 26.83 | A | N |
| ATOM | 1209 | CA | TYR | A | 158 | −15.597 | −54.138 | −1.874 | 1.00 | 29.99 | A | C |
| ATOM | 1210 | CB | TYR | A | 158 | −16.133 | −52.730 | −1.625 | 1.00 | 30.74 | A | C |
| ATOM | 1211 | CG | TYR | A | 158 | −16.036 | −51.870 | −2.854 | 1.00 | 37.88 | A | C |
| ATOM | 1212 | CD1 | TYR | A | 158 | −16.756 | −52.240 | −4.013 | 1.00 | 44.29 | A | C |
| ATOM | 1213 | CE1 | TYR | A | 158 | −16.708 | −51.472 | −5.186 | 1.00 | 46.05 | A | C |
| ATOM | 1214 | CZ | TYR | A | 158 | −15.952 | −50.285 | −5.215 | 1.00 | 46.84 | A | C |
| ATOM | 1215 | OH | TYR | A | 158 | −15.988 | −49.585 | −6.428 | 1.00 | 48.74 | A | O |
| ATOM | 1216 | CE2 | TYR | A | 158 | −15.219 | −49.856 | −4.067 | 1.00 | 41.11 | A | C |
| ATOM | 1217 | CD2 | TYR | A | 158 | −15.267 | −50.674 | −2.884 | 1.00 | 41.50 | A | C |
| ATOM | 1218 | C | TYR | A | 158 | −14.104 | −54.136 | −1.551 | 1.00 | 29.47 | A | C |
| ATOM | 1219 | O | TYR | A | 158 | −13.627 | −53.206 | −0.945 | 1.00 | 30.04 | A | O |
| ATOM | 1220 | N | GLU | A | 159 | −13.385 | −55.186 | −1.925 | 1.00 | 28.99 | A | N |
| ATOM | 1221 | CA | GLU | A | 159 | −11.976 | −55.272 | −1.641 | 1.00 | 28.82 | A | C |
| ATOM | 1222 | CB | GLU | A | 159 | −11.768 | −55.340 | −0.127 | 1.00 | 29.92 | A | C |
| ATOM | 1223 | CG | GLU | A | 159 | −12.306 | −56.655 | 0.403 | 1.00 | 37.72 | A | C |
| ATOM | 1224 | CD | GLU | A | 159 | −12.378 | −56.771 | 1.908 | 1.00 | 46.55 | A | C |
| ATOM | 1225 | OE1 | GLU | A | 159 | −12.816 | −57.862 | 2.360 | 1.00 | 50.45 | A | O |
| ATOM | 1226 | OE2 | GLU | A | 159 | −11.985 | −55.828 | 2.625 | 1.00 | 48.20 | A | O |
| ATOM | 1227 | C | GLU | A | 159 | −11.430 | −56.526 | −2.312 | 1.00 | 26.90 | A | C |
| ATOM | 1228 | O | GLU | A | 159 | −12.135 | −57.520 | −2.455 | 1.00 | 25.63 | A | O |
| ATOM | 1229 | N | ASN | A | 160 | −10.175 | −56.458 | −2.749 | 1.00 | 25.86 | A | N |
| ATOM | 1230 | CA | ASN | A | 160 | −9.476 | −57.603 | −3.289 | 1.00 | 24.72 | A | C |
| ATOM | 1231 | CB | ASN | A | 160 | −9.059 | −58.529 | −2.121 | 1.00 | 25.81 | A | C |
| ATOM | 1232 | CG | ASN | A | 160 | −8.197 | −57.765 | −1.025 | 1.00 | 27.32 | A | C |
| ATOM | 1233 | OD1 | ASN | A | 160 | −7.376 | −56.910 | −1.343 | 1.00 | 31.59 | A | O |
| ATOM | 1234 | ND2 | ASN | A | 160 | −8.415 | −58.075 | 0.224 | 1.00 | 26.78 | A | N |
| ATOM | 1235 | C | ASN | A | 160 | −10.273 | −58.290 | −4.423 | 1.00 | 24.15 | A | C |
| ATOM | 1236 | O | ASN | A | 160 | −10.980 | −57.617 | −5.193 | 1.00 | 24.63 | A | O |
| ATOM | 1237 | N | GLN | A | 161 | −10.171 | −59.597 | −4.565 | 1.00 | 23.15 | A | N |
| ATOM | 1238 | CA | GLN | A | 161 | −10.906 | −60.268 | −5.611 | 1.00 | 22.89 | A | C |
| ATOM | 1239 | CB | GLN | A | 161 | −10.611 | −61.755 | −5.633 | 1.00 | 23.27 | A | C |
| ATOM | 1240 | CG | GLN | A | 161 | −9.261 | −62.087 | −6.309 | 1.00 | 25.24 | A | C |
| ATOM | 1241 | CD | GLN | A | 161 | −8.807 | −63.524 | −6.096 | 1.00 | 24.45 | A | C |
| ATOM | 1242 | OE1 | GLN | A | 161 | −7.827 | −63.747 | −5.430 | 1.00 | 28.68 | A | O |
| ATOM | 1243 | NE2 | GLN | A | 161 | −9.507 | −64.484 | −6.661 | 1.00 | 23.13 | A | N |
| ATOM | 1244 | C | GLN | A | 161 | −12.409 | −60.032 | −5.552 | 1.00 | 22.22 | A | C |
| ATOM | 1245 | O | GLN | A | 161 | −12.998 | −59.792 | −6.604 | 1.00 | 23.02 | A | O |
| ATOM | 1246 | N | PRO | A | 162 | −13.039 | −60.085 | −4.344 | 1.00 | 20.98 | A | N |
| ATOM | 1247 | CA | PRO | A | 162 | −14.502 | −59.890 | −4.417 | 1.00 | 19.36 | A | C |
| ATOM | 1248 | CB | PRO | A | 162 | −14.975 | −60.120 | −2.987 | 1.00 | 18.81 | A | C |
| ATOM | 1249 | CG | PRO | A | 162 | −13.912 | −60.912 | −2.337 | 1.00 | 18.86 | A | C |
| ATOM | 1250 | CD | PRO | A | 162 | −12.606 | −60.623 | −3.033 | 1.00 | 19.79 | A | C |
| ATOM | 1251 | C | PRO | A | 162 | −14.857 | −58.470 | −4.924 | 1.00 | 19.83 | A | C |
| ATOM | 1252 | O | PRO | A | 162 | −15.809 | −58.341 | −5.670 | 1.00 | 19.72 | A | O |
| ATOM | 1253 | N | GLY | A | 163 | −14.068 | −57.436 | −4.605 | 1.00 | 18.23 | A | N |
| ATOM | 1254 | CA | GLY | A | 163 | −14.331 | −56.114 | −5.191 | 1.00 | 17.64 | A | C |
| ATOM | 1255 | C | GLY | A | 163 | −14.089 | −56.028 | −6.705 | 1.00 | 18.03 | A | C |
| ATOM | 1256 | O | GLY | A | 163 | −14.854 | −55.382 | −7.478 | 1.00 | 17.09 | A | O |
| ATOM | 1257 | N | ALA | A | 164 | −13.025 | −56.674 | −7.161 | 1.00 | 16.65 | A | N |
| ATOM | 1258 | CA | ALA | A | 164 | −12.727 | −56.654 | −8.580 | 1.00 | 16.33 | A | C |
| ATOM | 1259 | CB | ALA | A | 164 | −11.406 | −57.419 | −8.845 | 1.00 | 14.77 | A | C |
| ATOM | 1260 | C | ALA | A | 164 | −13.938 | −57.339 | −9.278 | 1.00 | 17.55 | A | C |
| ATOM | 1261 | O | ALA | A | 164 | −14.455 | −56.837 | −10.268 | 1.00 | 18.28 | A | O |
| ATOM | 1262 | N | LEU | A | 165 | −14.420 | −58.448 | −8.707 | 1.00 | 17.14 | A | N |
| ATOM | 1263 | CA | LEU | A | 165 | −15.608 | −59.097 | −9.196 | 1.00 | 17.89 | A | C |
| ATOM | 1264 | CB | LEU | A | 165 | −15.877 | −60.287 | −8.315 | 1.00 | 16.99 | A | C |
| ATOM | 1265 | CG | LEU | A | 165 | −15.729 | −61.677 | −8.900 | 1.00 | 19.04 | A | C |
| ATOM | 1266 | CD1 | LEU | A | 165 | −14.973 | −61.795 | −10.252 | 1.00 | 15.06 | A | C |
| ATOM | 1267 | CD2 | LEU | A | 165 | −15.198 | −62.613 | −7.856 | 1.00 | 13.97 | A | C |
| ATOM | 1268 | C | LEU | A | 165 | −16.847 | −58.169 | −9.210 | 1.00 | 18.52 | A | C |
| ATOM | 1269 | O | LEU | A | 165 | −17.639 | −58.204 | −10.166 | 1.00 | 19.03 | A | O |
| ATOM | 1270 | N | ASN | A | 166 | −16.998 | −57.340 | −8.177 | 1.00 | 17.89 | A | N |
| ATOM | 1271 | CA | ASN | A | 166 | −18.166 | −56.480 | −8.021 | 1.00 | 18.38 | A | C |
| ATOM | 1272 | CB | ASN | A | 166 | −18.079 | −55.783 | −6.670 | 1.00 | 18.80 | A | C |
| ATOM | 1273 | CG | ASN | A | 166 | −19.340 | −54.998 | −6.315 | 1.00 | 22.37 | A | C |
| ATOM | 1274 | OD1 | ASN | A | 166 | −19.843 | −55.100 | −5.194 | 1.00 | 27.99 | A | O |
| ATOM | 1275 | ND2 | ASN | A | 166 | −19.845 | −54.238 | −7.231 | 1.00 | 23.69 | A | N |
| ATOM | 1276 | C | ASN | A | 166 | −18.122 | −55.478 | −9.184 | 1.00 | 18.27 | A | C |
| ATOM | 1277 | O | ASN | A | 166 | −19.090 | −55.312 | −9.911 | 1.00 | 16.89 | A | O |
| ATOM | 1278 | N | GLU | A | 167 | −16.956 | −54.845 | −9.378 | 1.00 | 18.74 | A | N |
| ATOM | 1279 | CA | GLU | A | 167 | −16.739 | −53.911 | −10.509 | 1.00 | 19.05 | A | C |
| ATOM | 1280 | CB | GLU | A | 167 | −15.330 | −53.278 | −10.479 | 1.00 | 19.22 | A | C |
| ATOM | 1281 | CG | GLU | A | 167 | −15.166 | −52.277 | −9.341 | 1.00 | 18.04 | A | C |
| ATOM | 1282 | CD | GLU | A | 167 | −16.188 | −51.122 | −9.491 | 1.00 | 26.42 | A | C |
| ATOM | 1283 | OE1 | GLU | A | 167 | −16.420 | −50.721 | −10.651 | 1.00 | 23.23 | A | O |
| ATOM | 1284 | OE2 | GLU | A | 167 | −16.758 | −50.618 | −8.458 | 1.00 | 31.20 | A | O |
| ATOM | 1285 | C | GLU | A | 167 | −16.989 | −54.607 | −11.845 | 1.00 | 19.13 | A | C |
| ATOM | 1286 | O | GLU | A | 167 | −17.653 | −54.030 | −12.724 | 1.00 | 18.28 | A | O |
| ATOM | 1287 | N | SER | A | 168 | −16.521 | −55.853 | −11.965 | 1.00 | 18.00 | A | N |
| ATOM | 1288 | CA | SER | A | 168 | −16.599 | −56.564 | −13.230 | 1.00 | 18.98 | A | C |
| ATOM | 1289 | CB | SER | A | 168 | −15.698 | −57.800 | −13.208 | 1.00 | 19.87 | A | C |
| ATOM | 1290 | OG | SER | A | 168 | −15.795 | −58.557 | −14.402 | 1.00 | 21.32 | A | O |
| ATOM | 1291 | C | SER | A | 168 | −18.005 | −56.948 | −13.640 | 1.00 | 19.25 | A | C |
| ATOM | 1292 | O | SER | A | 168 | −18.367 | −56.783 | −14.817 | 1.00 | 20.90 | A | O |
| ATOM | 1293 | N | PHE | A | 169 | −18.794 | −57.485 | −12.710 | 1.00 | 18.94 | A | N |
| ATOM | 1294 | CA | PHE | A | 169 | −20.217 | −57.729 | −12.980 | 1.00 | 18.08 | A | C |
| ATOM | 1295 | CB | PHE | A | 169 | −20.900 | −58.365 | −11.790 | 1.00 | 17.52 | A | C |
| ATOM | 1296 | CG | PHE | A | 169 | −20.691 | −59.855 | −11.744 | 1.00 | 18.33 | A | C |
| ATOM | 1297 | CD1 | PHE | A | 169 | −21.599 | −60.729 | −12.369 | 1.00 | 20.04 | A | C |
| ATOM | 1298 | CE1 | PHE | A | 169 | −21.384 | −62.140 | −12.368 | 1.00 | 18.87 | A | C |
| ATOM | 1299 | CZ | PHE | A | 169 | −20.211 | −62.664 | −11.775 | 1.00 | 16.77 | A | C |
| ATOM | 1300 | CE2 | PHE | A | 169 | −19.287 | −61.777 | −11.188 | 1.00 | 15.44 | A | C |
| ATOM | 1301 | CD2 | PHE | A | 169 | −19.556 | −60.391 | −11.156 | 1.00 | 15.38 | A | C |
| ATOM | 1302 | C | PHE | A | 169 | −20.892 | −56.445 | −13.414 | 1.00 | 18.51 | A | C |
| ATOM | 1303 | O | PHE | A | 169 | −21.651 | −56.464 | −14.360 | 1.00 | 18.82 | A | O |
| ATOM | 1304 | N | SER | A | 170 | −20.574 | −55.317 | −12.778 | 1.00 | 18.28 | A | N |
| ATOM | 1305 | CA | SER | A | 170 | −21.139 | −54.034 | −13.214 | 1.00 | 18.86 | A | C |
| ATOM | 1306 | CB | SER | A | 170 | −20.808 | −52.909 | −12.207 | 1.00 | 19.58 | A | C |
| ATOM | 1307 | OG | SER | A | 170 | −21.680 | −52.939 | −11.063 | 1.00 | 18.44 | A | O |
| ATOM | 1308 | C | SER | A | 170 | −20.703 | −53.636 | −14.648 | 1.00 | 19.35 | A | C |
| ATOM | 1309 | O | SER | A | 170 | −21.548 | −53.179 | −15.481 | 1.00 | 19.84 | A | O |
| ATOM | 1310 | N | ASP | A | 171 | −19.419 | −53.836 | −14.973 | 1.00 | 18.01 | A | N |
| ATOM | 1311 | CA | ASP | A | 171 | −18.950 | −53.628 | −16.357 | 1.00 | 17.49 | A | C |
| ATOM | 1312 | CB | ASP | A | 171 | −17.426 | −53.709 | −16.481 | 1.00 | 17.01 | A | C |
| ATOM | 1313 | CG | ASP | A | 171 | −16.753 | −52.437 | −15.954 | 1.00 | 19.97 | A | C |
| ATOM | 1314 | OD1 | ASP | A | 171 | −17.486 | −51.457 | −15.784 | 1.00 | 23.37 | A | O |
| ATOM | 1315 | OD2 | ASP | A | 171 | −15.541 | −52.393 | −15.672 | 1.00 | 21.12 | A | O |
| ATOM | 1316 | C | ASP | A | 171 | −19.625 | −54.597 | −17.318 | 1.00 | 17.10 | A | C |
| ATOM | 1317 | O | ASP | A | 171 | −19.989 | −54.225 | −18.390 | 1.00 | 16.94 | A | O |
| ATOM | 1318 | N | VAL | A | 172 | −19.821 | −55.841 | −16.929 | 1.00 | 17.31 | A | N |
| ATOM | 1319 | CA | VAL | A | 172 | −20.311 | −56.797 | −17.915 | 1.00 | 17.03 | A | C |
| ATOM | 1320 | CB | VAL | A | 172 | −20.175 | −58.274 | −17.436 | 1.00 | 17.29 | A | C |
| ATOM | 1321 | CG1 | VAL | A | 172 | −20.952 | −59.218 | −18.390 | 1.00 | 10.63 | A | C |
| ATOM | 1322 | CG2 | VAL | A | 172 | −18.727 | −58.633 | −17.292 | 1.00 | 12.24 | A | C |
| ATOM | 1323 | C | VAL | A | 172 | −21.769 | −56.540 | −18.178 | 1.00 | 17.87 | A | C |
| ATOM | 1324 | O | VAL | A | 172 | −22.186 | −56.570 | −19.320 | 1.00 | 20.15 | A | O |
| ATOM | 1325 | N | PHE | A | 173 | −22.566 | −56.286 | −17.139 | 1.00 | 17.61 | A | N |
| ATOM | 1326 | CA | PHE | A | 173 | −23.938 | −55.837 | −17.384 | 1.00 | 16.74 | A | C |
| ATOM | 1327 | CB | PHE | A | 173 | −24.788 | −55.923 | −16.129 | 1.00 | 15.90 | A | C |
| ATOM | 1328 | CG | PHE | A | 173 | −25.200 | −57.319 | −15.836 | 1.00 | 16.63 | A | C |
| ATOM | 1329 | CD1 | PHE | A | 173 | −26.192 | −57.945 | −16.615 | 1.00 | 15.84 | A | C |
| ATOM | 1330 | CE1 | PHE | A | 173 | −26.541 | −59.316 | −16.394 | 1.00 | 15.38 | A | C |
| ATOM | 1331 | CZ | PHE | A | 173 | −25.920 | −60.041 | −15.400 | 1.00 | 12.78 | A | C |
| ATOM | 1332 | CE2 | PHE | A | 173 | −24.915 | −59.420 | −14.624 | 1.00 | 15.12 | A | C |
| ATOM | 1333 | CD2 | PHE | A | 173 | −24.547 | −58.063 | −14.864 | 1.00 | 14.07 | A | C |
| ATOM | 1334 | C | PHE | A | 173 | −23.980 | −54.476 | −18.083 | 1.00 | 16.64 | A | C |
| ATOM | 1335 | O | PHE | A | 173 | −24.859 | −54.210 | −18.881 | 1.00 | 16.87 | A | O |
| ATOM | 1336 | N | GLY | A | 174 | −23.012 | −53.618 | −17.829 | 1.00 | 16.88 | A | N |
| ATOM | 1337 | CA | GLY | A | 174 | −22.910 | −52.412 | −18.676 | 1.00 | 17.01 | A | C |
| ATOM | 1338 | C | GLY | A | 174 | −22.824 | −52.773 | −20.129 | 1.00 | 15.87 | A | C |
| ATOM | 1339 | O | GLY | A | 174 | −23.639 | −52.364 | −20.946 | 1.00 | 16.39 | A | O |
| ATOM | 1340 | N | TYR | A | 175 | −21.850 | −53.597 | −20.440 | 1.00 | 16.21 | A | N |
| ATOM | 1341 | CA | TYR | A | 175 | −21.672 | −54.101 | −21.818 | 1.00 | 16.33 | A | C |
| ATOM | 1342 | CB | TYR | A | 175 | −20.505 | −55.135 | −21.929 | 1.00 | 15.56 | A | C |
| ATOM | 1343 | CG | TYR | A | 175 | −20.634 | −55.868 | −23.242 | 1.00 | 16.30 | A | C |
| ATOM | 1344 | CD1 | TYR | A | 175 | −20.244 | −55.242 | −24.446 | 1.00 | 15.97 | A | C |
| ATOM | 1345 | CE1 | TYR | A | 175 | −20.412 | −55.874 | −25.660 | 1.00 | 15.05 | A | C |
| ATOM | 1346 | CZ | TYR | A | 175 | −20.993 | −57.103 | −25.692 | 1.00 | 12.27 | A | C |
| ATOM | 1347 | OH | TYR | A | 175 | −21.122 | −57.668 | −26.889 | 1.00 | 19.05 | A | O |
| ATOM | 1348 | CE2 | TYR | A | 175 | −21.447 | −57.760 | −24.541 | 1.00 | 15.27 | A | C |
| ATOM | 1349 | CD2 | TYR | A | 175 | −21.266 | −57.140 | −23.315 | 1.00 | 16.32 | A | C |
| ATOM | 1350 | C | TYR | A | 175 | −22.940 | −54.727 | −22.397 | 1.00 | 16.62 | A | C |
| ATOM | 1351 | O | TYR | A | 175 | −23.255 | −54.468 | −23.558 | 1.00 | 17.16 | A | O |
| ATOM | 1352 | N | PHE | A | 176 | −23.635 | −55.601 | −21.634 | 1.00 | 16.39 | A | N |
| ATOM | 1353 | CA | PHE | A | 176 | −24.897 | −56.208 | −22.145 | 1.00 | 16.88 | A | C |
| ATOM | 1354 | CB | PHE | A | 176 | −25.520 | −57.206 | −21.179 | 1.00 | 15.95 | A | C |
| ATOM | 1355 | CG | PHE | A | 176 | −24.676 | −58.450 | −20.932 | 1.00 | 17.51 | A | C |
| ATOM | 1356 | CD1 | PHE | A | 176 | −23.993 | −59.092 | −21.990 | 1.00 | 17.21 | A | C |
| ATOM | 1357 | CE1 | PHE | A | 176 | −23.200 | −60.253 | −21.776 | 1.00 | 14.72 | A | C |
| ATOM | 1358 | CZ | PHE | A | 176 | −23.112 | −60.805 | −20.542 | 1.00 | 13.49 | A | C |
| ATOM | 1359 | CE2 | PHE | A | 176 | −23.805 | −60.198 | −19.455 | 1.00 | 16.22 | A | C |
| ATOM | 1360 | CD2 | PHE | A | 176 | −24.583 | −59.005 | −19.657 | 1.00 | 12.99 | A | C |
| ATOM | 1361 | C | PHE | A | 176 | −25.969 | −55.159 | −22.503 | 1.00 | 18.52 | A | C |
| ATOM | 1362 | O | PHE | A | 176 | −26.918 | −55.487 | −23.240 | 1.00 | 19.58 | A | O |
| ATOM | 1363 | N | ASN | A | 177 | −25.867 | −53.935 | −21.969 | 1.00 | 18.66 | A | N |
| ATOM | 1364 | CA | ASN | A | 177 | −26.812 | −52.883 | −22.349 | 1.00 | 20.84 | A | C |
| ATOM | 1365 | CB | ASN | A | 177 | −27.074 | −51.973 | −21.158 | 1.00 | 21.25 | A | C |
| ATOM | 1366 | CG | ASN | A | 177 | −28.132 | −52.547 | −20.240 | 1.00 | 23.82 | A | C |
| ATOM | 1367 | OD1 | ASN | A | 177 | −29.337 | −52.292 | −20.447 | 1.00 | 26.73 | A | O |
| ATOM | 1368 | ND2 | ASN | A | 177 | −27.713 | −53.430 | −19.304 | 1.00 | 19.14 | A | N |
| ATOM | 1369 | C | ASN | A | 177 | −26.349 | −52.026 | −23.528 | 1.00 | 22.09 | A | C |
| ATOM | 1370 | O | ASN | A | 177 | −27.098 | −51.180 | −24.026 | 1.00 | 22.65 | A | O |
| ATOM | 1371 | N | ASP | A | 178 | −25.096 | −52.227 | −23.949 | 1.00 | 22.56 | A | N |
| ATOM | 1372 | CA | ASP | A | 178 | −24.585 | −51.613 | −25.167 | 1.00 | 22.81 | A | C |
| ATOM | 1373 | CB | ASP | A | 178 | −23.717 | −50.419 | −24.795 | 1.00 | 22.90 | A | C |
| ATOM | 1374 | CG | ASP | A | 178 | −23.211 | −49.690 | −26.009 | 1.00 | 24.04 | A | C |
| ATOM | 1375 | OD1 | ASP | A | 178 | −23.707 | −50.024 | −27.136 | 1.00 | 23.24 | A | O |
| ATOM | 1376 | OD2 | ASP | A | 178 | −22.320 | −48.827 | −25.821 | 1.00 | 23.69 | A | O |
| ATOM | 1377 | C | ASP | A | 178 | −23.756 | −52.589 | −25.997 | 1.00 | 22.85 | A | C |
| ATOM | 1378 | O | ASP | A | 178 | −22.551 | −52.517 | −25.957 | 1.00 | 22.00 | A | O |
| ATOM | 1379 | N | THR | A | 179 | −24.373 | −53.519 | −26.733 | 1.00 | 23.82 | A | N |
| ATOM | 1380 | CA | THR | A | 179 | −23.587 | −54.582 | −27.357 | 1.00 | 23.81 | A | C |
| ATOM | 1381 | CB | THR | A | 179 | −24.382 | −55.843 | −27.549 | 1.00 | 23.60 | A | C |
| ATOM | 1382 | OG1 | THR | A | 179 | −25.444 | −55.583 | −28.454 | 1.00 | 22.97 | A | O |
| ATOM | 1383 | CG2 | THR | A | 179 | −24.988 | −56.319 | −26.217 | 1.00 | 23.48 | A | C |
| ATOM | 1384 | C | THR | A | 179 | −22.874 | −54.197 | −28.652 | 1.00 | 25.94 | A | C |
| ATOM | 1385 | O | THR | A | 179 | −22.231 | −55.060 | −29.276 | 1.00 | 28.95 | A | O |
| ATOM | 1386 | N | GLU | A | 180 | −22.905 | −52.916 | −29.011 | 1.00 | 25.67 | A | N |
| ATOM | 1387 | CA | GLU | A | 180 | −22.389 | −52.446 | −30.283 | 1.00 | 26.89 | A | C |
| ATOM | 1388 | CB | GLU | A | 180 | −23.122 | −51.171 | −30.761 | 1.00 | 26.98 | A | C |
| ATOM | 1389 | CG | GLU | A | 180 | −24.670 | −51.330 | −30.837 | 1.00 | 35.87 | A | C |
| ATOM | 1390 | CD | GLU | A | 180 | −25.179 | −52.083 | −32.100 | 1.00 | 42.93 | A | C |
| ATOM | 1391 | OE1 | GLU | A | 180 | −25.040 | −51.571 | −33.253 | 1.00 | 44.91 | A | O |
| ATOM | 1392 | OE2 | GLU | A | 180 | −25.746 | −53.192 | −31.912 | 1.00 | 46.19 | A | O |
| ATOM | 1393 | C | GLU | A | 180 | −20.879 | −52.186 | −30.191 | 1.00 | 25.23 | A | C |
| ATOM | 1394 | O | GLU | A | 180 | −20.239 | −52.040 | −31.212 | 1.00 | 23.83 | A | O |
| ATOM | 1395 | N | ASP | A | 181 | −20.348 | −52.112 | −28.967 | 1.00 | 23.52 | A | N |
| ATOM | 1396 | CA | ASP | A | 181 | −18.938 | −51.832 | −28.760 | 1.00 | 22.58 | A | C |
| ATOM | 1397 | CB | ASP | A | 181 | −18.655 | −50.340 | −28.850 | 1.00 | 21.01 | A | C |
| ATOM | 1398 | CG | ASP | A | 181 | −19.321 | −49.522 | −27.710 | 1.00 | 22.57 | A | C |
| ATOM | 1399 | OD1 | ASP | A | 181 | −18.851 | −49.707 | −26.552 | 1.00 | 18.34 | A | O |
| ATOM | 1400 | OD2 | ASP | A | 181 | −20.243 | −48.665 | −27.982 | 1.00 | 16.82 | A | O |
| ATOM | 1401 | C | ASP | A | 181 | −18.475 | −52.504 | −27.448 | 1.00 | 22.44 | A | C |
| ATOM | 1402 | O | ASP | A | 181 | −19.320 | −52.946 | −26.690 | 1.00 | 23.47 | A | O |
| ATOM | 1403 | N | TRP | A | 182 | −17.171 | −52.683 | −27.223 | 1.00 | 20.84 | A | N |
| ATOM | 1404 | CA | TRP | A | 182 | −16.721 | −53.385 | −26.028 | 1.00 | 20.90 | A | C |
| ATOM | 1405 | CB | TRP | A | 182 | −15.592 | −54.408 | −26.332 | 1.00 | 20.47 | A | C |
| ATOM | 1406 | CG | TRP | A | 182 | −15.970 | −55.461 | −27.334 | 1.00 | 22.13 | A | C |
| ATOM | 1407 | CD1 | TRP | A | 182 | −15.669 | −55.448 | −28.650 | 1.00 | 22.17 | A | C |
| ATOM | 1408 | NE1 | TRP | A | 182 | −16.166 | −56.554 | −29.262 | 1.00 | 23.53 | A | N |
| ATOM | 1409 | CE2 | TRP | A | 182 | −16.785 | −57.337 | −28.332 | 1.00 | 21.98 | A | C |
| ATOM | 1410 | CD2 | TRP | A | 182 | −16.686 | −56.673 | −27.101 | 1.00 | 20.26 | A | C |
| ATOM | 1411 | CE3 | TRP | A | 182 | −17.205 | −57.286 | −25.966 | 1.00 | 23.99 | A | C |
| ATOM | 1412 | CZ3 | TRP | A | 182 | −17.855 | −58.507 | −26.101 | 1.00 | 22.34 | A | C |
| ATOM | 1413 | CH2 | TRP | A | 182 | −17.953 | −59.143 | −27.362 | 1.00 | 21.91 | A | C |
| ATOM | 1414 | CZ2 | TRP | A | 182 | −17.419 | −58.583 | −28.483 | 1.00 | 20.01 | A | C |
| ATOM | 1415 | C | TRP | A | 182 | −16.261 | −52.469 | −24.900 | 1.00 | 20.55 | A | C |
| ATOM | 1416 | O | TRP | A | 182 | −15.597 | −52.916 | −23.924 | 1.00 | 20.79 | A | O |
| ATOM | 1417 | N | ASP | A | 183 | −16.572 | −51.197 | −25.029 | 1.00 | 20.24 | A | N |
| ATOM | 1418 | CA | ASP | A | 183 | −16.041 | −50.196 | −24.082 | 1.00 | 20.11 | A | C |
| ATOM | 1419 | CB | ASP | A | 183 | −15.603 | −48.958 | −24.837 | 1.00 | 22.04 | A | C |
| ATOM | 1420 | CG | ASP | A | 183 | −14.519 | −49.243 | −25.853 | 1.00 | 22.53 | A | C |
| ATOM | 1421 | OD1 | ASP | A | 183 | −13.631 | −50.058 | −25.566 | 1.00 | 22.42 | A | O |
| ATOM | 1422 | OD2 | ASP | A | 183 | −14.559 | −48.640 | −26.931 | 1.00 | 28.89 | A | O |
| ATOM | 1423 | C | ASP | A | 183 | −17.103 | −49.771 | −23.098 | 1.00 | 19.38 | A | C |
| ATOM | 1424 | O | ASP | A | 183 | −18.305 | −49.994 | −23.280 | 1.00 | 18.81 | A | O |
| ATOM | 1425 | N | ILE | A | 184 | −16.634 | −49.198 | −22.016 | 1.00 | 19.19 | A | N |
| ATOM | 1426 | CA | ILE | A | 184 | −17.472 | −48.656 | −21.008 | 1.00 | 17.88 | A | C |
| ATOM | 1427 | CB | ILE | A | 184 | −17.207 | −49.273 | −19.633 | 1.00 | 17.96 | A | C |
| ATOM | 1428 | CG1 | ILE | A | 184 | −17.542 | −50.770 | −19.598 | 1.00 | 15.40 | A | C |
| ATOM | 1429 | CD1 | ILE | A | 184 | −19.003 | −51.161 | −20.100 | 1.00 | 11.93 | A | C |
| ATOM | 1430 | CG2 | ILE | A | 184 | −17.982 | −48.453 | −18.531 | 1.00 | 17.15 | A | C |
| ATOM | 1431 | C | ILE | A | 184 | −17.137 | −47.196 | −20.953 | 1.00 | 19.24 | A | C |
| ATOM | 1432 | O | ILE | A | 184 | −15.951 | −46.792 | −20.858 | 1.00 | 18.87 | A | O |
| ATOM | 1433 | N | GLY | A | 185 | −18.188 | −46.389 | −21.023 | 1.00 | 21.21 | A | N |
| ATOM | 1434 | CA | GLY | A | 185 | −18.053 | −44.959 | −20.843 | 1.00 | 21.03 | A | C |
| ATOM | 1435 | C | GLY | A | 185 | −17.689 | −44.215 | −22.100 | 1.00 | 22.01 | A | C |
| ATOM | 1436 | O | GLY | A | 185 | −17.408 | −43.045 | −21.967 | 1.00 | 24.52 | A | O |
| ATOM | 1437 | N | GLU | A | 186 | −17.654 | −44.826 | −23.304 | 1.00 | 22.73 | A | N |
| ATOM | 1438 | CA | GLU | A | 186 | −17.326 | −44.039 | −24.564 | 1.00 | 23.37 | A | C |
| ATOM | 1439 | CB | GLU | A | 186 | −17.879 | −44.589 | −25.903 | 1.00 | 22.89 | A | C |
| ATOM | 1440 | CG | GLU | A | 186 | −18.056 | −45.939 | −26.167 | 1.00 | 25.12 | A | C |
| ATOM | 1441 | CD | GLU | A | 186 | −19.138 | −46.652 | −25.424 | 1.00 | 22.33 | A | C |
| ATOM | 1442 | OE1 | GLU | A | 186 | −20.284 | −46.845 | −25.960 | 1.00 | 21.68 | A | O |
| ATOM | 1443 | OE2 | GLU | A | 186 | −18.737 | −47.213 | −24.397 | 1.00 | 22.18 | A | O |
| ATOM | 1444 | C | GLU | A | 186 | −18.099 | −42.696 | −24.596 | 1.00 | 22.60 | A | C |
| ATOM | 1445 | O | GLU | A | 186 | −17.618 | −41.754 | −25.165 | 1.00 | 20.62 | A | O |
| ATOM | 1446 | N | ASP | A | 187 | −19.369 | −42.720 | −24.167 | 1.00 | 22.32 | A | N |
| ATOM | 1447 | CA | ASP | A | 187 | −20.271 | −41.596 | −24.391 | 1.00 | 23.51 | A | C |
| ATOM | 1448 | CB | ASP | A | 187 | −21.713 | −42.035 | −24.268 | 1.00 | 23.08 | A | C |
| ATOM | 1449 | CG | ASP | A | 187 | −22.113 | −42.937 | −25.376 | 1.00 | 25.22 | A | C |
| ATOM | 1450 | OD1 | ASP | A | 187 | −22.001 | −42.509 | −26.563 | 1.00 | 30.20 | A | O |
| ATOM | 1451 | OD2 | ASP | A | 187 | −22.524 | −44.071 | −25.076 | 1.00 | 23.02 | A | O |
| ATOM | 1452 | C | ASP | A | 187 | −20.027 | −40.439 | −23.465 | 1.00 | 23.38 | A | C |
| ATOM | 1453 | O | ASP | A | 187 | −20.566 | −39.384 | −23.691 | 1.00 | 23.91 | A | O |
| ATOM | 1454 | N | ILE | A | 188 | −19.201 | −40.619 | −22.438 | 1.00 | 23.41 | A | N |
| ATOM | 1455 | CA | ILE | A | 188 | −18.961 | −39.496 | −21.533 | 1.00 | 23.94 | A | C |
| ATOM | 1456 | CB | ILE | A | 188 | −19.559 | −39.732 | −20.159 | 1.00 | 23.51 | A | C |
| ATOM | 1457 | CG1 | ILE | A | 188 | −18.854 | −40.951 | −19.557 | 1.00 | 23.55 | A | C |
| ATOM | 1458 | CD1 | ILE | A | 188 | −19.426 | −41.402 | −18.274 | 1.00 | 32.06 | A | C |
| ATOM | 1459 | CG2 | ILE | A | 188 | −21.052 | −39.892 | −20.310 | 1.00 | 22.40 | A | C |
| ATOM | 1460 | C | ILE | A | 188 | −17.497 | −39.168 | −21.316 | 1.00 | 23.80 | A | C |
| ATOM | 1461 | O | ILE | A | 188 | −17.230 | −38.262 | −20.549 | 1.00 | 23.43 | A | O |
| ATOM | 1462 | N | THR | A | 189 | −16.566 | −39.889 | −21.943 | 1.00 | 24.69 | A | N |
| ATOM | 1463 | CA | THR | A | 189 | −15.119 | −39.571 | −21.732 | 1.00 | 26.63 | A | C |
| ATOM | 1464 | CB | THR | A | 189 | −14.162 | −40.770 | −21.846 | 1.00 | 26.40 | A | C |
| ATOM | 1465 | OG1 | THR | A | 189 | −14.423 | −41.478 | −23.060 | 1.00 | 27.13 | A | O |
| ATOM | 1466 | CG2 | THR | A | 189 | −14.341 | −41.690 | −20.690 | 1.00 | 23.01 | A | C |
| ATOM | 1467 | C | THR | A | 189 | −14.682 | −38.528 | −22.714 | 1.00 | 28.64 | A | C |
| ATOM | 1468 | O | THR | A | 189 | −15.058 | −38.577 | −23.901 | 1.00 | 29.97 | A | O |
| ATOM | 1469 | N | ILE | A | 190 | −13.958 | −37.531 | −22.231 | 1.00 | 30.38 | A | N |
| ATOM | 1470 | CA | ILE | A | 190 | −13.570 | −36.450 | −23.129 | 1.00 | 32.28 | A | C |
| ATOM | 1471 | CB | ILE | A | 190 | −13.879 | −35.064 | −22.516 | 1.00 | 34.01 | A | C |
| ATOM | 1472 | CG1 | ILE | A | 190 | −15.423 | −34.848 | −22.441 | 1.00 | 32.91 | A | C |
| ATOM | 1473 | CD1 | ILE | A | 190 | −15.854 | −33.566 | −21.723 | 1.00 | 31.28 | A | C |
| ATOM | 1474 | CG2 | ILE | A | 190 | −13.060 | −33.922 | −23.296 | 1.00 | 32.95 | A | C |
| ATOM | 1475 | C | ILE | A | 190 | −12.094 | −36.542 | −23.563 | 1.00 | 32.73 | A | C |
| ATOM | 1476 | O | ILE | A | 190 | −11.803 | −36.448 | −24.738 | 1.00 | 32.45 | A | O |
| ATOM | 1477 | N | SER | A | 191 | −11.169 | −36.751 | −22.627 | 1.00 | 33.19 | A | N |
| ATOM | 1478 | CA | SER | A | 191 | −9.775 | −36.805 | −23.025 | 1.00 | 33.49 | A | C |
| ATOM | 1479 | CB | SER | A | 191 | −8.920 | −36.253 | −21.920 | 1.00 | 33.14 | A | C |
| ATOM | 1480 | OG | SER | A | 191 | −9.041 | −37.100 | −20.809 | 1.00 | 36.97 | A | O |
| ATOM | 1481 | C | SER | A | 191 | −9.306 | −38.202 | −23.382 | 1.00 | 34.18 | A | C |
| ATOM | 1482 | O | SER | A | 191 | −8.091 | −38.453 | −23.467 | 1.00 | 35.06 | A | O |
| ATOM | 1483 | N | GLN | A | 192 | −10.228 | −39.146 | −23.573 | 1.00 | 33.35 | A | N |
| ATOM | 1484 | CA | GLN | A | 192 | −9.783 | −40.462 | −23.989 | 1.00 | 32.23 | A | C |
| ATOM | 1485 | CB | GLN | A | 192 | −9.101 | −41.218 | −22.838 | 1.00 | 33.39 | A | C |
| ATOM | 1486 | CG | GLN | A | 192 | −9.988 | −41.561 | −21.702 | 1.00 | 38.13 | A | C |
| ATOM | 1487 | CD | GLN | A | 192 | −9.247 | −42.255 | −20.544 | 1.00 | 43.66 | A | C |
| ATOM | 1488 | OE1 | GLN | A | 192 | −8.231 | −41.748 | −20.087 | 1.00 | 47.31 | A | O |
| ATOM | 1489 | NE2 | GLN | A | 192 | −9.778 | −43.396 | −20.052 | 1.00 | 41.63 | A | N |
| ATOM | 1490 | C | GLN | A | 192 | −10.937 | −41.186 | −24.580 | 1.00 | 30.01 | A | C |
| ATOM | 1491 | O | GLN | A | 192 | −12.071 | −40.805 | −24.353 | 1.00 | 30.89 | A | O |
| ATOM | 1492 | N | PRO | A | 193 | −10.678 | −42.212 | −25.372 | 1.00 | 27.78 | A | N |
| ATOM | 1493 | CA | PRO | A | 193 | −11.870 | −42.698 | −26.109 | 1.00 | 26.45 | A | C |
| ATOM | 1494 | CB | PRO | A | 193 | −11.270 | −43.631 | −27.207 | 1.00 | 26.27 | A | C |
| ATOM | 1495 | CG | PRO | A | 193 | −9.795 | −43.281 | −27.236 | 1.00 | 27.16 | A | C |
| ATOM | 1496 | CD | PRO | A | 193 | −9.408 | −42.778 | −25.858 | 1.00 | 26.09 | A | C |
| ATOM | 1497 | C | PRO | A | 193 | −12.923 | −43.471 | −25.273 | 1.00 | 25.90 | A | C |
| ATOM | 1498 | O | PRO | A | 193 | −14.023 | −43.682 | −25.773 | 1.00 | 27.07 | A | O |
| ATOM | 1499 | N | ALA | A | 194 | −12.574 | −43.950 | −24.072 | 1.00 | 24.70 | A | N |
| ATOM | 1500 | CA | ALA | A | 194 | −13.440 | −44.762 | −23.204 | 1.00 | 23.36 | A | C |
| ATOM | 1501 | CB | ALA | A | 194 | −13.575 | −46.154 | −23.742 | 1.00 | 22.19 | A | C |
| ATOM | 1502 | C | ALA | A | 194 | −12.818 | −44.815 | −21.812 | 1.00 | 24.00 | A | C |
| ATOM | 1503 | O | ALA | A | 194 | −11.698 | −44.385 | −21.593 | 1.00 | 24.86 | A | O |
| ATOM | 1504 | N | LEU | A | 195 | −13.539 | −45.349 | −20.857 | 1.00 | 23.81 | A | N |
| ATOM | 1505 | CA | LEU | A | 195 | −13.018 | −45.520 | −19.534 | 1.00 | 24.41 | A | C |
| ATOM | 1506 | CB | LEU | A | 195 | −14.229 | −45.511 | −18.651 | 1.00 | 25.95 | A | C |
| ATOM | 1507 | CG | LEU | A | 195 | −14.275 | −44.996 | −17.236 | 1.00 | 30.68 | A | C |
| ATOM | 1508 | CD1 | LEU | A | 195 | −13.317 | −43.838 | −17.055 | 1.00 | 33.65 | A | C |
| ATOM | 1509 | CD2 | LEU | A | 195 | −15.726 | −44.586 | −16.969 | 1.00 | 30.18 | A | C |
| ATOM | 1510 | C | LEU | A | 195 | −12.270 | −46.863 | −19.403 | 1.00 | 24.08 | A | C |
| ATOM | 1511 | O | LEU | A | 195 | −11.192 | −46.935 | −18.804 | 1.00 | 23.41 | A | O |
| ATOM | 1512 | N | ARG | A | 196 | −12.841 | −47.922 | −20.014 | 1.00 | 23.67 | A | N |
| ATOM | 1513 | CA | ARG | A | 196 | −12.370 | −49.296 | −19.891 | 1.00 | 22.76 | A | C |
| ATOM | 1514 | CB | ARG | A | 196 | −12.961 | −49.963 | −18.629 | 1.00 | 23.02 | A | C |
| ATOM | 1515 | CG | ARG | A | 196 | −12.367 | −49.426 | −17.342 | 1.00 | 21.73 | A | C |
| ATOM | 1516 | CD | ARG | A | 196 | −12.813 | −50.233 | −16.158 | 1.00 | 23.25 | A | C |
| ATOM | 1517 | NE | ARG | A | 196 | −14.264 | −50.087 | −15.937 | 1.00 | 24.59 | A | N |
| ATOM | 1518 | CZ | ARG | A | 196 | −14.837 | −49.069 | −15.282 | 1.00 | 20.86 | A | C |
| ATOM | 1519 | NH1 | ARG | A | 196 | −14.092 | −48.100 | −14.791 | 1.00 | 17.04 | A | N |
| ATOM | 1520 | NH2 | ARG | A | 196 | −16.159 | −49.037 | −15.109 | 1.00 | 21.02 | A | N |
| ATOM | 1521 | C | ARG | A | 196 | −12.807 | −50.069 | −21.114 | 1.00 | 22.52 | A | C |
| ATOM | 1522 | O | ARG | A | 196 | −13.819 | −49.727 | −21.723 | 1.00 | 21.99 | A | O |
| ATOM | 1523 | N | SER | A | 197 | −12.018 | −51.090 | −21.485 | 1.00 | 22.05 | A | N |
| ATOM | 1524 | CA | SER | A | 197 | −12.362 | −51.996 | −22.573 | 1.00 | 21.00 | A | C |
| ATOM | 1525 | CB | SER | A | 197 | −11.229 | −52.025 | −23.613 | 1.00 | 20.95 | A | C |
| ATOM | 1526 | OG | SER | A | 197 | −11.566 | −52.922 | −24.687 | 1.00 | 18.92 | A | O |
| ATOM | 1527 | C | SER | A | 197 | −12.544 | −53.422 | −22.020 | 1.00 | 21.27 | A | C |
| ATOM | 1528 | O | SER | A | 197 | −11.708 | −53.882 | −21.198 | 1.00 | 21.38 | A | O |
| ATOM | 1529 | N | LEU | A | 198 | −13.585 | −54.117 | −22.484 | 1.00 | 19.82 | A | N |
| ATOM | 1530 | CA | LEU | A | 198 | −13.686 | −55.557 | −22.248 | 1.00 | 19.79 | A | C |
| ATOM | 1531 | CB | LEU | A | 198 | −15.145 | −56.034 | −22.319 | 1.00 | 19.75 | A | C |
| ATOM | 1532 | CG | LEU | A | 198 | −15.904 | −56.087 | −20.985 | 1.00 | 21.07 | A | C |
| ATOM | 1533 | CD1 | LEU | A | 198 | −16.178 | −54.692 | −20.481 | 1.00 | 21.88 | A | C |
| ATOM | 1534 | CD2 | LEU | A | 198 | −17.157 | −56.815 | −21.298 | 1.00 | 21.91 | A | C |
| ATOM | 1535 | C | LEU | A | 198 | −12.806 | −56.434 | −23.166 | 1.00 | 19.74 | A | C |
| ATOM | 1536 | O | LEU | A | 198 | −12.344 | −57.508 | −22.740 | 1.00 | 19.53 | A | O |
| ATOM | 1537 | N | SER | A | 199 | −12.609 | −56.013 | −24.424 | 1.00 | 19.06 | A | N |
| ATOM | 1538 | CA | SER | A | 199 | −11.884 | −56.819 | −25.395 | 1.00 | 19.27 | A | C |
| ATOM | 1539 | CB | SER | A | 199 | −12.216 | −56.441 | −26.825 | 1.00 | 18.43 | A | C |
| ATOM | 1540 | OG | SER | A | 199 | −11.972 | −55.055 | −27.037 | 1.00 | 20.70 | A | O |
| ATOM | 1541 | C | SER | A | 199 | −10.433 | −56.637 | −25.153 | 1.00 | 19.83 | A | C |
| ATOM | 1542 | O | SER | A | 199 | −9.659 | −57.529 | −25.360 | 1.00 | 20.84 | A | O |
| ATOM | 1543 | N | ASN | A | 200 | −10.032 | −55.478 | −24.689 | 1.00 | 21.41 | A | N |
| ATOM | 1544 | CA | ASN | A | 200 | −8.634 | −55.334 | −24.362 | 1.00 | 21.87 | A | C |
| ATOM | 1545 | CB | ASN | A | 200 | −7.791 | −54.832 | −25.537 | 1.00 | 21.65 | A | C |
| ATOM | 1546 | CG | ASN | A | 200 | −6.249 | −54.889 | −25.231 | 1.00 | 27.06 | A | C |
| ATOM | 1547 | OD1 | ASN | A | 200 | −5.475 | −54.393 | −26.049 | 1.00 | 30.41 | A | O |
| ATOM | 1548 | ND2 | ASN | A | 200 | −5.809 | −55.501 | −24.039 | 1.00 | 24.34 | A | N |
| ATOM | 1549 | C | ASN | A | 200 | −8.422 | −54.438 | −23.181 | 1.00 | 21.54 | A | C |
| ATOM | 1550 | O | ASN | A | 200 | −8.158 | −53.226 | −23.359 | 1.00 | 22.88 | A | O |
| ATOM | 1551 | N | PRO | A | 201 | −8.456 | −55.035 | −21.969 | 1.00 | 20.76 | A | N |
| ATOM | 1552 | CA | PRO | A | 201 | −8.386 | −54.221 | −20.767 | 1.00 | 19.97 | A | C |
| ATOM | 1553 | CB | PRO | A | 201 | −8.583 | −55.233 | −19.649 | 1.00 | 20.04 | A | C |
| ATOM | 1554 | CG | PRO | A | 201 | −9.418 | −56.297 | −20.256 | 1.00 | 17.27 | A | C |
| ATOM | 1555 | CD | PRO | A | 201 | −8.940 | −56.400 | −21.672 | 1.00 | 20.21 | A | C |
| ATOM | 1556 | C | PRO | A | 201 | −7.119 | −53.460 | −20.605 | 1.00 | 20.08 | A | C |
| ATOM | 1557 | O | PRO | A | 201 | −7.154 | −52.398 | −19.957 | 1.00 | 20.34 | A | O |
| ATOM | 1558 | N | THR | A | 202 | −6.013 | −53.952 | −21.165 | 1.00 | 20.30 | A | N |
| ATOM | 1559 | CA | THR | A | 202 | −4.687 | −53.266 | −20.959 | 1.00 | 20.87 | A | C |
| ATOM | 1560 | CB | THR | A | 202 | −3.423 | −54.140 | −21.428 | 1.00 | 21.44 | A | C |
| ATOM | 1561 | OG1 | THR | A | 202 | −3.517 | −54.431 | −22.817 | 1.00 | 20.58 | A | O |
| ATOM | 1562 | CG2 | THR | A | 202 | −3.344 | −55.458 | −20.668 | 1.00 | 20.68 | A | C |
| ATOM | 1563 | C | THR | A | 202 | −4.593 | −51.904 | −21.646 | 1.00 | 21.36 | A | C |
| ATOM | 1564 | O | THR | A | 202 | −3.792 | −51.104 | −21.320 | 1.00 | 22.47 | A | O |
| ATOM | 1565 | N | LYS | A | 203 | −5.450 | −51.641 | −22.592 | 1.00 | 22.15 | A | N |
| ATOM | 1566 | CA | LYS | A | 203 | −5.507 | −50.383 | −23.264 | 1.00 | 23.44 | A | C |
| ATOM | 1567 | CB | LYS | A | 203 | −6.710 | −50.552 | −24.145 | 1.00 | 23.94 | A | C |
| ATOM | 1568 | CG | LYS | A | 203 | −6.858 | −49.774 | −25.356 | 1.00 | 27.38 | A | C |
| ATOM | 1569 | CD | LYS | A | 203 | −8.155 | −50.373 | −25.996 | 1.00 | 33.35 | A | C |
| ATOM | 1570 | CE | LYS | A | 203 | −8.589 | −49.721 | −27.299 | 1.00 | 34.69 | A | C |
| ATOM | 1571 | NZ | LYS | A | 203 | −9.750 | −50.462 | −27.851 | 1.00 | 28.98 | A | N |
| ATOM | 1572 | C | LYS | A | 203 | −5.760 | −49.217 | −22.295 | 1.00 | 24.00 | A | C |
| ATOM | 1573 | O | LYS | A | 203 | −5.411 | −48.078 | −22.598 | 1.00 | 24.08 | A | O |
| ATOM | 1574 | N | TYR | A | 204 | −6.439 | −49.493 | −21.165 | 1.00 | 23.80 | A | N |
| ATOM | 1575 | CA | TYR | A | 204 | −6.757 | −48.477 | −20.131 | 1.00 | 22.53 | A | C |
| ATOM | 1576 | CB | TYR | A | 204 | −8.279 | −48.163 | −19.987 | 1.00 | 21.98 | A | C |
| ATOM | 1577 | CG | TYR | A | 204 | −8.883 | −47.674 | −21.292 | 1.00 | 21.51 | A | C |
| ATOM | 1578 | CD1 | TYR | A | 204 | −8.557 | −46.403 | −21.813 | 1.00 | 22.54 | A | C |
| ATOM | 1579 | CE1 | TYR | A | 204 | −9.011 | −45.973 | −23.036 | 1.00 | 20.99 | A | C |
| ATOM | 1580 | CZ | TYR | A | 204 | −9.841 | −46.786 | −23.781 | 1.00 | 26.90 | A | C |
| ATOM | 1581 | OH | TYR | A | 204 | −10.293 | −46.348 | −25.000 | 1.00 | 29.43 | A | O |
| ATOM | 1582 | CE2 | TYR | A | 204 | −10.178 | −48.064 | −23.333 | 1.00 | 25.65 | A | C |
| ATOM | 1583 | CD2 | TYR | A | 204 | −9.663 | −48.503 | −22.069 | 1.00 | 24.69 | A | C |
| ATOM | 1584 | C | TYR | A | 204 | −6.159 | −48.892 | −18.820 | 1.00 | 22.27 | A | C |
| ATOM | 1585 | O | TYR | A | 204 | −6.671 | −48.521 | −17.794 | 1.00 | 21.04 | A | O |
| ATOM | 1586 | N | GLY | A | 205 | −5.058 | −49.650 | −18.870 | 1.00 | 22.10 | A | N |
| ATOM | 1587 | CA | GLY | A | 205 | −4.262 | −49.918 | −17.675 | 1.00 | 22.18 | A | C |
| ATOM | 1588 | C | GLY | A | 205 | −4.809 | −50.989 | −16.730 | 1.00 | 22.79 | A | C |
| ATOM | 1589 | O | GLY | A | 205 | −4.430 | −50.991 | −15.582 | 1.00 | 23.60 | A | O |
| ATOM | 1590 | N | GLN | A | 206 | −5.648 | −51.923 | −17.219 | 1.00 | 21.26 | A | N |
| ATOM | 1591 | CA | GLN | A | 206 | −6.107 | −53.065 | −16.447 | 1.00 | 19.18 | A | C |
| ATOM | 1592 | CB | GLN | A | 206 | −7.630 | −53.235 | −16.524 | 1.00 | 18.15 | A | C |
| ATOM | 1593 | CG | GLN | A | 206 | −8.377 | −52.048 | −16.028 | 1.00 | 17.17 | A | C |
| ATOM | 1594 | CD | GLN | A | 206 | −9.904 | −52.209 | −16.063 | 1.00 | 21.79 | A | C |
| ATOM | 1595 | OE1 | GLN | A | 206 | −10.523 | −52.330 | −17.135 | 1.00 | 18.29 | A | O |
| ATOM | 1596 | NE2 | GLN | A | 206 | −10.525 | −52.152 | −14.883 | 1.00 | 20.04 | A | N |
| ATOM | 1597 | C | GLN | A | 206 | −5.450 | −54.321 | −16.974 | 1.00 | 19.36 | A | C |
| ATOM | 1598 | O | GLN | A | 206 | −5.357 | −54.518 | −18.186 | 1.00 | 19.20 | A | O |
| ATOM | 1599 | N | PRO | A | 207 | −5.012 | −55.210 | −16.056 | 1.00 | 19.13 | A | N |
| ATOM | 1600 | CA | PRO | A | 207 | −4.493 | −56.519 | −16.465 | 1.00 | 17.37 | A | C |
| ATOM | 1601 | CB | PRO | A | 207 | −4.123 | −57.151 | −15.119 | 1.00 | 17.34 | A | C |
| ATOM | 1602 | CG | PRO | A | 207 | −4.994 | −56.447 | −14.112 | 1.00 | 16.68 | A | C |
| ATOM | 1603 | CD | PRO | A | 207 | −5.103 | −55.070 | −14.581 | 1.00 | 17.24 | A | C |
| ATOM | 1604 | C | PRO | A | 207 | −5.609 | −57.340 | −17.104 | 1.00 | 19.09 | A | C |
| ATOM | 1605 | O | PRO | A | 207 | −6.823 | −57.179 | −16.786 | 1.00 | 18.40 | A | O |
| ATOM | 1606 | N | ASP | A | 208 | −5.242 | −58.267 | −17.982 | 1.00 | 19.79 | A | N |
| ATOM | 1607 | CA | ASP | A | 208 | −6.287 | −59.043 | −18.617 | 1.00 | 19.30 | A | C |
| ATOM | 1608 | CB | ASP | A | 208 | −6.405 | −58.653 | −20.118 | 1.00 | 20.08 | A | C |
| ATOM | 1609 | CG | ASP | A | 208 | −5.187 | −59.120 | −20.946 | 1.00 | 21.13 | A | C |
| ATOM | 1610 | OD1 | ASP | A | 208 | −4.286 | −59.708 | −20.379 | 1.00 | 20.02 | A | O |
| ATOM | 1611 | OD2 | ASP | A | 208 | −5.120 | −58.915 | −22.167 | 1.00 | 29.06 | A | O |
| ATOM | 1612 | C | ASP | A | 208 | −5.899 | −60.513 | −18.392 | 1.00 | 19.37 | A | C |
| ATOM | 1613 | O | ASP | A | 208 | −6.395 | −61.433 | −19.078 | 1.00 | 19.60 | A | O |
| ATOM | 1614 | N | ASN | A | 209 | −4.972 | −60.727 | −17.455 | 1.00 | 18.32 | A | N |
| ATOM | 1615 | CA | ASN | A | 209 | −4.466 | −62.070 | −17.169 | 1.00 | 17.32 | A | C |
| ATOM | 1616 | CB | ASN | A | 209 | −3.246 | −62.433 | −18.071 | 1.00 | 15.81 | A | C |
| ATOM | 1617 | CG | ASN | A | 209 | −2.821 | −63.952 | −17.937 | 1.00 | 18.79 | A | C |
| ATOM | 1618 | OD1 | ASN | A | 209 | −2.149 | −64.383 | −16.941 | 1.00 | 17.96 | A | O |
| ATOM | 1619 | ND2 | ASN | A | 209 | −3.226 | −64.760 | −18.938 | 1.00 | 16.61 | A | N |
| ATOM | 1620 | C | ASN | A | 209 | −4.108 | −62.160 | −15.700 | 1.00 | 16.81 | A | C |
| ATOM | 1621 | O | ASN | A | 209 | −3.563 | −61.188 | −15.078 | 1.00 | 18.22 | A | O |
| ATOM | 1622 | N | PHE | A | 210 | −4.332 | −63.314 | −15.130 | 1.00 | 15.27 | A | N |
| ATOM | 1623 | CA | PHE | A | 210 | −4.271 | −63.352 | −13.695 | 1.00 | 16.31 | A | C |
| ATOM | 1624 | CB | PHE | A | 210 | −5.023 | −64.587 | −13.159 | 1.00 | 15.05 | A | C |
| ATOM | 1625 | CG | PHE | A | 210 | −5.035 | −64.702 | −11.635 | 1.00 | 18.41 | A | C |
| ATOM | 1626 | CD1 | PHE | A | 210 | −5.798 | −63.830 | −10.847 | 1.00 | 17.35 | A | C |
| ATOM | 1627 | CE1 | PHE | A | 210 | −5.823 | −63.930 | −9.476 | 1.00 | 14.09 | A | C |
| ATOM | 1628 | CZ | PHE | A | 210 | −5.139 | −64.956 | −8.821 | 1.00 | 18.23 | A | C |
| ATOM | 1629 | CE2 | PHE | A | 210 | −4.398 | −65.857 | −9.542 | 1.00 | 22.42 | A | C |
| ATOM | 1630 | CD2 | PHE | A | 210 | −4.331 | −65.726 | −10.982 | 1.00 | 22.85 | A | C |
| ATOM | 1631 | C | PHE | A | 210 | −2.805 | −63.235 | −13.180 | 1.00 | 16.82 | A | C |
| ATOM | 1632 | O | PHE | A | 210 | −2.584 | −62.770 | −12.078 | 1.00 | 18.49 | A | O |
| ATOM | 1633 | N | LYS | A | 211 | −1.820 | −63.679 | −13.963 | 1.00 | 16.71 | A | N |
| ATOM | 1634 | CA | LYS | A | 211 | −0.456 | −63.522 | −13.587 | 1.00 | 16.11 | A | C |
| ATOM | 1635 | CB | LYS | A | 211 | 0.520 | −64.063 | −14.628 | 1.00 | 15.62 | A | C |
| ATOM | 1636 | CG | LYS | A | 211 | 0.349 | −65.592 | −14.816 | 1.00 | 18.60 | A | C |
| ATOM | 1637 | CD | LYS | A | 211 | 1.446 | −66.227 | −15.594 | 1.00 | 18.76 | A | C |
| ATOM | 1638 | CE | LYS | A | 211 | 1.391 | −65.839 | −17.100 | 1.00 | 22.01 | A | C |
| ATOM | 1639 | NZ | LYS | A | 211 | 0.075 | −66.030 | −17.786 | 1.00 | 23.18 | A | N |
| ATOM | 1640 | C | LYS | A | 211 | −0.191 | −62.056 | −13.365 | 1.00 | 16.71 | A | C |
| ATOM | 1641 | O | LYS | A | 211 | 0.733 | −61.781 | −12.666 | 1.00 | 18.56 | A | O |
| ATOM | 1642 | N | ASN | A | 212 | −0.989 | −61.139 | −13.917 | 1.00 | 16.09 | A | N |
| ATOM | 1643 | CA | ASN | A | 212 | −0.705 | −59.723 | −13.783 | 1.00 | 17.44 | A | C |
| ATOM | 1644 | CB | ASN | A | 212 | −0.759 | −59.008 | −15.168 | 1.00 | 15.91 | A | C |
| ATOM | 1645 | CG | ASN | A | 212 | 0.176 | −59.639 | −16.156 | 1.00 | 18.08 | A | C |
| ATOM | 1646 | OD1 | ASN | A | 212 | 1.287 | −60.089 | −15.768 | 1.00 | 20.31 | A | O |
| ATOM | 1647 | ND2 | ASN | A | 212 | −0.239 | −59.719 | −17.437 | 1.00 | 13.44 | A | N |
| ATOM | 1648 | C | ASN | A | 212 | −1.638 | −58.995 | −12.839 | 1.00 | 18.72 | A | C |
| ATOM | 1649 | O | ASN | A | 212 | −1.699 | −57.779 | −12.907 | 1.00 | 20.55 | A | O |
| ATOM | 1650 | N | TYR | A | 213 | −2.417 | −59.718 | −12.041 | 1.00 | 20.14 | A | N |
| ATOM | 1651 | CA | TYR | A | 213 | −3.203 | −59.181 | −10.941 | 1.00 | 22.18 | A | C |
| ATOM | 1652 | CB | TYR | A | 213 | −3.678 | −60.382 | −10.076 | 1.00 | 21.74 | A | C |
| ATOM | 1653 | CG | TYR | A | 213 | −4.569 | −60.001 | −8.895 | 1.00 | 22.28 | A | C |
| ATOM | 1654 | CD1 | TYR | A | 213 | −5.960 | −60.034 | −9.009 | 1.00 | 21.52 | A | C |
| ATOM | 1655 | CE1 | TYR | A | 213 | −6.771 | −59.682 | −7.945 | 1.00 | 25.23 | A | C |
| ATOM | 1656 | CZ | TYR | A | 213 | −6.199 | −59.285 | −6.716 | 1.00 | 24.59 | A | C |
| ATOM | 1657 | OH | TYR | A | 213 | −7.033 | −58.937 | −5.676 | 1.00 | 25.57 | A | O |
| ATOM | 1658 | CE2 | TYR | A | 213 | −4.841 | −59.229 | −6.572 | 1.00 | 20.07 | A | C |
| ATOM | 1659 | CD2 | TYR | A | 213 | −4.020 | −59.585 | −7.679 | 1.00 | 23.48 | A | C |
| ATOM | 1660 | C | TYR | A | 213 | −2.325 | −58.231 | −10.096 | 1.00 | 23.86 | A | C |
| ATOM | 1661 | O | TYR | A | 213 | −1.247 | −58.629 | −9.696 | 1.00 | 24.04 | A | O |
| ATOM | 1662 | N | LYS | A | 214 | −2.769 | −56.999 | −9.862 | 1.00 | 26.34 | A | N |
| ATOM | 1663 | CA | LYS | A | 214 | −1.988 | −55.999 | −9.139 | 1.00 | 29.23 | A | C |
| ATOM | 1664 | CB | LYS | A | 214 | −2.379 | −54.571 | −9.560 | 1.00 | 28.88 | A | C |
| ATOM | 1665 | CG | LYS | A | 214 | −1.891 | −54.173 | −10.948 | 1.00 | 34.77 | A | C |
| ATOM | 1666 | CD | LYS | A | 214 | −0.472 | −54.771 | −11.307 | 1.00 | 42.59 | A | C |
| ATOM | 1667 | CE | LYS | A | 214 | 0.018 | −54.429 | −12.759 | 1.00 | 46.77 | A | C |
| ATOM | 1668 | NZ | LYS | A | 214 | −0.563 | −55.320 | −13.814 | 1.00 | 44.75 | A | N |
| ATOM | 1669 | C | LYS | A | 214 | −2.153 | −56.159 | −7.638 | 1.00 | 30.36 | A | C |
| ATOM | 1670 | O | LYS | A | 214 | −3.239 | −55.937 | −7.112 | 1.00 | 29.56 | A | O |
| ATOM | 1671 | N | ASN | A | 215 | −1.087 | −56.456 | −6.949 | 1.00 | 32.59 | A | N |
| ATOM | 1672 | CA | ASN | A | 215 | −1.146 | −56.618 | −5.534 | 1.00 | 35.97 | A | C |
| ATOM | 1673 | CB | ASN | A | 215 | −0.024 | −57.504 | −5.121 | 1.00 | 37.03 | A | C |
| ATOM | 1674 | CG | ASN | A | 215 | −0.447 | −58.524 | −4.182 | 1.00 | 45.30 | A | C |
| ATOM | 1675 | OD1 | ASN | A | 215 | −1.581 | −58.963 | −4.202 | 1.00 | 55.68 | A | O |
| ATOM | 1676 | ND2 | ASN | A | 215 | 0.460 | −58.920 | −3.309 | 1.00 | 48.95 | A | N |
| ATOM | 1677 | C | ASN | A | 215 | −1.097 | −55.333 | −4.736 | 1.00 | 35.60 | A | C |
| ATOM | 1678 | O | ASN | A | 215 | −0.160 | −55.071 | −4.071 | 1.00 | 37.05 | A | O |
| ATOM | 1679 | N | LEU | A | 216 | −2.142 | −54.545 | −4.803 | 1.00 | 35.58 | A | N |
| ATOM | 1680 | CA | LEU | A | 216 | −2.245 | −53.342 | −4.006 | 1.00 | 34.88 | A | C |
| ATOM | 1681 | CB | LEU | A | 216 | −3.192 | −52.367 | −4.683 | 1.00 | 33.33 | A | C |
| ATOM | 1682 | CG | LEU | A | 216 | −2.645 | −51.966 | −6.042 | 1.00 | 33.49 | A | C |
| ATOM | 1683 | CD1 | LEU | A | 216 | −1.328 | −51.060 | −5.941 | 1.00 | 30.51 | A | C |
| ATOM | 1684 | CD2 | LEU | A | 216 | −3.737 | −51.333 | −6.956 | 1.00 | 30.71 | A | C |
| ATOM | 1685 | C | LEU | A | 216 | −2.672 | −53.627 | −2.544 | 1.00 | 35.63 | A | C |
| ATOM | 1686 | O | LEU | A | 216 | −3.253 | −54.695 | −2.238 | 1.00 | 34.93 | A | O |
| ATOM | 1687 | N | PRO | A | 217 | −2.313 | −52.703 | −1.617 | 1.00 | 36.04 | A | N |
| ATOM | 1688 | CA | PRO | A | 217 | −2.773 | −52.946 | −0.251 | 1.00 | 34.97 | A | C |
| ATOM | 1689 | CB | PRO | A | 217 | −1.826 | −52.085 | 0.598 | 1.00 | 34.61 | A | C |
| ATOM | 1690 | CG | PRO | A | 217 | −1.400 | −50.968 | −0.374 | 1.00 | 36.58 | A | C |
| ATOM | 1691 | CD | PRO | A | 217 | −1.221 | −51.699 | −1.661 | 1.00 | 36.55 | A | C |
| ATOM | 1692 | C | PRO | A | 217 | −4.268 | −52.572 | −0.092 | 1.00 | 34.04 | A | C |
| ATOM | 1693 | O | PRO | A | 217 | −4.858 | −51.753 | −0.843 | 1.00 | 33.03 | A | O |
| ATOM | 1694 | N | ASN | A | 218 | −4.876 | −53.226 | 0.879 | 1.00 | 32.61 | A | N |
| ATOM | 1695 | CA | ASN | A | 218 | −6.270 | −53.043 | 1.126 | 1.00 | 31.31 | A | C |
| ATOM | 1696 | CB | ASN | A | 218 | −6.832 | −54.318 | 1.763 | 1.00 | 29.93 | A | C |
| ATOM | 1697 | CG | ASN | A | 218 | −8.318 | −54.301 | 1.829 | 1.00 | 27.40 | A | C |
| ATOM | 1698 | OD1 | ASN | A | 218 | −8.947 | −53.358 | 1.320 | 1.00 | 26.18 | A | O |
| ATOM | 1699 | ND2 | ASN | A | 218 | −8.911 | −55.309 | 2.494 | 1.00 | 19.83 | A | N |
| ATOM | 1700 | C | ASN | A | 218 | −6.520 | −51.815 | 1.987 | 1.00 | 31.62 | A | C |
| ATOM | 1701 | O | ASN | A | 218 | −6.790 | −51.939 | 3.153 | 1.00 | 31.01 | A | O |
| ATOM | 1702 | N | THR | A | 219 | −6.450 | −50.630 | 1.382 | 1.00 | 32.99 | A | N |
| ATOM | 1703 | CA | THR | A | 219 | −6.485 | −49.327 | 2.093 | 1.00 | 33.63 | A | C |
| ATOM | 1704 | CB | THR | A | 219 | −5.053 | −48.809 | 2.427 | 1.00 | 34.29 | A | C |
| ATOM | 1705 | OG1 | THR | A | 219 | −4.364 | −48.497 | 1.191 | 1.00 | 36.98 | A | O |
| ATOM | 1706 | CG2 | THR | A | 219 | −4.243 | −49.832 | 3.250 | 1.00 | 33.05 | A | C |
| ATOM | 1707 | C | THR | A | 219 | −7.013 | −48.315 | 1.104 | 1.00 | 33.34 | A | C |
| ATOM | 1708 | O | THR | A | 219 | −6.959 | −48.567 | −0.089 | 1.00 | 33.10 | A | O |
| ATOM | 1709 | N | PRO | A | 220 | −7.484 | −47.146 | 1.562 | 1.00 | 33.55 | A | N |
| ATOM | 1710 | CA | PRO | A | 220 | −7.957 | −46.213 | 0.490 | 1.00 | 34.11 | A | C |
| ATOM | 1711 | CB | PRO | A | 220 | −8.394 | −44.943 | 1.263 | 1.00 | 33.67 | A | C |
| ATOM | 1712 | CG | PRO | A | 220 | −8.401 | −45.295 | 2.659 | 1.00 | 32.53 | A | C |
| ATOM | 1713 | CD | PRO | A | 220 | −7.794 | −46.666 | 2.911 | 1.00 | 32.53 | A | C |
| ATOM | 1714 | C | PRO | A | 220 | −6.879 | −45.880 | −0.591 | 1.00 | 34.21 | A | C |
| ATOM | 1715 | O | PRO | A | 220 | −7.205 | −45.756 | −1.779 | 1.00 | 34.70 | A | O |
| ATOM | 1716 | N | ALA | A | 221 | −5.621 | −45.743 | −0.178 | 1.00 | 33.57 | A | N |
| ATOM | 1717 | CA | ALA | A | 221 | −4.528 | −45.392 | −1.112 | 1.00 | 33.92 | A | C |
| ATOM | 1718 | CB | ALA | A | 221 | −3.157 | −45.124 | −0.344 | 1.00 | 33.52 | A | C |
| ATOM | 1719 | C | ALA | A | 221 | −4.353 | −46.519 | −2.148 | 1.00 | 33.79 | A | C |
| ATOM | 1720 | O | ALA | A | 221 | −4.193 | −46.233 | −3.326 | 1.00 | 35.18 | A | O |
| ATOM | 1721 | N | GLY | A | 222 | −4.375 | −47.779 | −1.694 | 1.00 | 32.11 | A | N |
| ATOM | 1722 | CA | GLY | A | 222 | −4.400 | −48.959 | −2.550 | 1.00 | 30.09 | A | C |
| ATOM | 1723 | C | GLY | A | 222 | −5.687 | −49.271 | −3.346 | 1.00 | 29.61 | A | C |
| ATOM | 1724 | O | GLY | A | 222 | −5.760 | −50.308 | −4.010 | 1.00 | 28.33 | A | O |
| ATOM | 1725 | N | ASP | A | 223 | −6.692 | −48.385 | −3.349 | 1.00 | 28.45 | A | N |
| ATOM | 1726 | CA | ASP | A | 223 | −7.980 | −48.764 | −4.003 | 1.00 | 28.35 | A | C |
| ATOM | 1727 | CB | ASP | A | 223 | −7.787 | −48.802 | −5.520 | 1.00 | 27.61 | A | C |
| ATOM | 1728 | CG | ASP | A | 223 | −9.098 | −48.814 | −6.281 | 1.00 | 30.68 | A | C |
| ATOM | 1729 | OD1 | ASP | A | 223 | −9.101 | −49.277 | −7.448 | 1.00 | 32.09 | A | O |
| ATOM | 1730 | OD2 | ASP | A | 223 | −10.127 | −48.359 | −5.724 | 1.00 | 36.19 | A | O |
| ATOM | 1731 | C | ASP | A | 223 | −8.530 | −50.124 | −3.459 | 1.00 | 27.02 | A | C |
| ATOM | 1732 | O | ASP | A | 223 | −8.979 | −51.018 | −4.195 | 1.00 | 25.89 | A | O |
| ATOM | 1733 | N | TYR | A | 224 | −8.429 | −50.276 | −2.148 | 1.00 | 26.54 | A | N |
| ATOM | 1734 | CA | TYR | A | 224 | −8.894 | −51.485 | −1.451 | 1.00 | 26.72 | A | C |
| ATOM | 1735 | CB | TYR | A | 224 | −10.425 | −51.446 | −1.256 | 1.00 | 25.59 | A | C |
| ATOM | 1736 | CG | TYR | A | 224 | −10.803 | −50.163 | −0.604 | 1.00 | 25.61 | A | C |
| ATOM | 1737 | CD1 | TYR | A | 224 | −10.450 | −49.932 | 0.719 | 1.00 | 26.00 | A | C |
| ATOM | 1738 | CE1 | TYR | A | 224 | −10.759 | −48.717 | 1.364 | 1.00 | 26.61 | A | C |
| ATOM | 1739 | CZ | TYR | A | 224 | −11.426 | −47.718 | 0.670 | 1.00 | 28.65 | A | C |
| ATOM | 1740 | OH | TYR | A | 224 | −11.695 | −46.564 | 1.364 | 1.00 | 31.55 | A | O |
| ATOM | 1741 | CE2 | TYR | A | 224 | −11.778 | −47.884 | −0.680 | 1.00 | 24.77 | A | C |
| ATOM | 1742 | CD2 | TYR | A | 224 | −11.448 | −49.132 | −1.315 | 1.00 | 26.18 | A | C |
| ATOM | 1743 | C | TYR | A | 224 | −8.455 | −52.742 | −2.151 | 1.00 | 26.55 | A | C |
| ATOM | 1744 | O | TYR | A | 224 | −9.255 | −53.616 | −2.386 | 1.00 | 27.97 | A | O |
| ATOM | 1745 | N | GLY | A | 225 | −7.174 | −52.825 | −2.484 | 1.00 | 26.70 | A | N |
| ATOM | 1746 | CA | GLY | A | 225 | −6.604 | −54.038 | −3.046 | 1.00 | 24.99 | A | C |
| ATOM | 1747 | C | GLY | A | 225 | −6.686 | −54.032 | −4.576 | 1.00 | 24.95 | A | C |
| ATOM | 1748 | O | GLY | A | 225 | −6.602 | −55.087 | −5.173 | 1.00 | 25.97 | A | O |
| ATOM | 1749 | N | GLY | A | 226 | −6.881 | −52.879 | −5.206 | 1.00 | 22.96 | A | N |
| ATOM | 1750 | CA | GLY | A | 226 | −6.876 | −52.794 | −6.658 | 1.00 | 23.27 | A | C |
| ATOM | 1751 | C | GLY | A | 226 | −8.208 | −53.065 | −7.380 | 1.00 | 23.87 | A | C |
| ATOM | 1752 | O | GLY | A | 226 | −8.258 | −53.513 | −8.540 | 1.00 | 24.79 | A | O |
| ATOM | 1753 | N | VAL | A | 227 | −9.290 | −52.745 | −6.710 | 1.00 | 23.04 | A | N |
| ATOM | 1754 | CA | VAL | A | 227 | −10.625 | −53.162 | −7.073 | 1.00 | 22.69 | A | C |
| ATOM | 1755 | CB | VAL | A | 227 | −11.553 | −52.701 | −5.895 | 1.00 | 23.09 | A | C |
| ATOM | 1756 | CG1 | VAL | A | 227 | −12.761 | −52.005 | −6.310 | 1.00 | 20.90 | A | C |
| ATOM | 1757 | CG2 | VAL | A | 227 | −11.769 | −53.815 | −4.873 | 1.00 | 22.17 | A | C |
| ATOM | 1758 | C | VAL | A | 227 | −11.033 | −52.668 | −8.467 | 1.00 | 23.44 | A | C |
| ATOM | 1759 | O | VAL | A | 227 | −11.670 | −53.398 | −9.206 | 1.00 | 23.58 | A | O |
| ATOM | 1760 | N | HIS | A | 228 | −10.608 | −51.461 | −8.843 | 1.00 | 23.13 | A | N |
| ATOM | 1761 | CA | HIS | A | 228 | −10.921 | −50.876 | −10.133 | 1.00 | 23.87 | A | C |
| ATOM | 1762 | CB | HIS | A | 228 | −10.907 | −49.340 | −10.010 | 1.00 | 24.16 | A | C |
| ATOM | 1763 | CG | HIS | A | 228 | −12.028 | −48.829 | −9.173 | 1.00 | 29.12 | A | C |
| ATOM | 1764 | ND1 | HIS | A | 228 | −11.909 | −48.651 | −7.803 | 1.00 | 27.92 | A | N |
| ATOM | 1765 | CE1 | HIS | A | 228 | −13.076 | −48.251 | −7.325 | 1.00 | 29.37 | A | C |
| ATOM | 1766 | NE2 | HIS | A | 228 | −13.939 | −48.169 | −8.327 | 1.00 | 29.73 | A | N |
| ATOM | 1767 | CD2 | HIS | A | 228 | −13.315 | −48.522 | −9.498 | 1.00 | 26.55 | A | C |
| ATOM | 1768 | C | HIS | A | 228 | −9.905 | −51.230 | −11.202 | 1.00 | 23.76 | A | C |
| ATOM | 1769 | O | HIS | A | 228 | −10.149 | −50.923 | −12.372 | 1.00 | 23.21 | A | O |
| ATOM | 1770 | N | THR | A | 229 | −8.734 | −51.751 | −10.819 | 1.00 | 22.54 | A | N |
| ATOM | 1771 | CA | THR | A | 229 | −7.830 | −52.122 | −11.846 | 1.00 | 23.69 | A | C |
| ATOM | 1772 | CB | THR | A | 229 | −6.389 | −51.542 | −11.723 | 1.00 | 25.20 | A | C |
| ATOM | 1773 | OG1 | THR | A | 229 | −5.400 | −52.578 | −11.619 | 1.00 | 28.21 | A | O |
| ATOM | 1774 | CG2 | THR | A | 229 | −6.279 | −50.571 | −10.632 | 1.00 | 24.20 | A | C |
| ATOM | 1775 | C | THR | A | 229 | −7.947 | −53.613 | −12.134 | 1.00 | 23.03 | A | C |
| ATOM | 1776 | O | THR | A | 229 | −8.152 | −53.971 | −13.277 | 1.00 | 22.94 | A | O |
| ATOM | 1777 | N | ASN | A | 230 | −7.940 | −54.455 | −11.100 | 1.00 | 22.15 | A | N |
| ATOM | 1778 | CA | ASN | A | 230 | −8.058 | −55.904 | −11.271 | 1.00 | 21.10 | A | C |
| ATOM | 1779 | CB | ASN | A | 230 | −7.726 | −56.621 | −9.992 | 1.00 | 19.61 | A | C |
| ATOM | 1780 | CG | ASN | A | 230 | −6.306 | −56.361 | −9.574 | 1.00 | 22.93 | A | C |
| ATOM | 1781 | OD1 | ASN | A | 230 | −5.397 | −56.225 | −10.416 | 1.00 | 25.85 | A | O |
| ATOM | 1782 | ND2 | ASN | A | 230 | −6.084 | −56.287 | −8.289 | 1.00 | 24.86 | A | N |
| ATOM | 1783 | C | ASN | A | 230 | −9.353 | −56.449 | −11.870 | 1.00 | 20.93 | A | C |
| ATOM | 1784 | O | ASN | A | 230 | −9.417 | −57.628 | −12.232 | 1.00 | 21.10 | A | O |
| ATOM | 1785 | N | SER | A | 231 | −10.353 | −55.601 | −12.040 | 1.00 | 19.41 | A | N |
| ATOM | 1786 | CA | SER | A | 231 | −11.594 | −56.083 | −12.603 | 1.00 | 19.21 | A | C |
| ATOM | 1787 | CB | SER | A | 231 | −12.638 | −54.982 | −12.525 | 1.00 | 19.11 | A | C |
| ATOM | 1788 | OG | SER | A | 231 | −12.088 | −53.760 | −13.008 | 1.00 | 20.47 | A | O |
| ATOM | 1789 | C | SER | A | 231 | −11.362 | −56.457 | −14.053 | 1.00 | 19.87 | A | C |
| ATOM | 1790 | O | SER | A | 231 | −12.201 | −57.153 | −14.688 | 1.00 | 22.04 | A | O |
| ATOM | 1791 | N | GLY | A | 232 | −10.239 | −56.000 | −14.607 | 1.00 | 18.34 | A | N |
| ATOM | 1792 | CA | GLY | A | 232 | −9.908 | −56.335 | −15.989 | 1.00 | 17.71 | A | C |
| ATOM | 1793 | C | GLY | A | 232 | −9.746 | −57.827 | −16.229 | 1.00 | 17.62 | A | C |
| ATOM | 1794 | O | GLY | A | 232 | −9.950 | −58.302 | −17.370 | 1.00 | 18.95 | A | O |
| ATOM | 1795 | N | ILE | A | 233 | −9.433 | −58.587 | −15.169 | 1.00 | 16.84 | A | N |
| ATOM | 1796 | CA | ILE | A | 233 | −9.226 | −60.005 | −15.346 | 1.00 | 15.89 | A | C |
| ATOM | 1797 | CB | ILE | A | 233 | −8.370 | −60.622 | −14.217 | 1.00 | 16.51 | A | C |
| ATOM | 1798 | CG1 | ILE | A | 233 | −6.945 | −60.055 | −14.326 | 1.00 | 16.48 | A | C |
| ATOM | 1799 | CD1 | ILE | A | 233 | −6.233 | −59.984 | −12.977 | 1.00 | 17.92 | A | C |
| ATOM | 1800 | CG2 | ILE | A | 233 | −8.355 | −62.182 | −14.255 | 1.00 | 12.46 | A | C |
| ATOM | 1801 | C | ILE | A | 233 | −10.523 | −60.704 | −15.579 | 1.00 | 16.17 | A | C |
| ATOM | 1802 | O | ILE | A | 233 | −10.652 | −61.379 | −16.578 | 1.00 | 16.06 | A | O |
| ATOM | 1803 | N | PRO | A | 234 | −11.503 | −60.554 | −14.674 | 1.00 | 16.91 | A | N |
| ATOM | 1804 | CA | PRO | A | 234 | −12.803 | −61.186 | −14.959 | 1.00 | 16.88 | A | C |
| ATOM | 1805 | CB | PRO | A | 234 | −13.588 | −61.028 | −13.663 | 1.00 | 17.47 | A | C |
| ATOM | 1806 | CG | PRO | A | 234 | −12.874 | −59.914 | −12.897 | 1.00 | 17.44 | A | C |
| ATOM | 1807 | CD | PRO | A | 234 | −11.413 | −60.111 | −13.282 | 1.00 | 16.92 | A | C |
| ATOM | 1808 | C | PRO | A | 234 | −13.540 | −60.494 | −16.103 | 1.00 | 17.34 | A | C |
| ATOM | 1809 | O | PRO | A | 234 | −14.370 | −61.164 | −16.763 | 1.00 | 17.89 | A | O |
| ATOM | 1810 | N | ASN | A | 235 | −13.213 | −59.230 | −16.421 | 1.00 | 16.49 | A | N |
| ATOM | 1811 | CA | ASN | A | 235 | −13.880 | −58.615 | −17.615 | 1.00 | 16.69 | A | C |
| ATOM | 1812 | CB | ASN | A | 235 | −13.571 | −57.150 | −17.771 | 1.00 | 16.50 | A | C |
| ATOM | 1813 | CG | ASN | A | 235 | −14.452 | −56.279 | −16.873 | 1.00 | 17.42 | A | C |
| ATOM | 1814 | OD1 | ASN | A | 235 | −15.346 | −56.800 | −16.177 | 1.00 | 16.73 | A | O |
| ATOM | 1815 | ND2 | ASN | A | 235 | −14.217 | −54.948 | −16.890 | 1.00 | 14.95 | A | N |
| ATOM | 1816 | C | ASN | A | 235 | −13.467 | −59.334 | −18.849 | 1.00 | 17.37 | A | C |
| ATOM | 1817 | O | ASN | A | 235 | −14.303 | −59.692 | −19.699 | 1.00 | 17.84 | A | O |
| ATOM | 1818 | N | LYS | A | 236 | −12.159 | −59.618 | −18.927 | 1.00 | 17.83 | A | N |
| ATOM | 1819 | CA | LYS | A | 236 | −11.641 | −60.409 | −20.021 | 1.00 | 16.30 | A | C |
| ATOM | 1820 | CB | LYS | A | 236 | −10.143 | −60.479 | −19.933 | 1.00 | 16.14 | A | C |
| ATOM | 1821 | CG | LYS | A | 236 | −9.520 | −61.075 | −21.131 | 1.00 | 13.45 | A | C |
| ATOM | 1822 | CD | LYS | A | 236 | −9.677 | −60.098 | −22.339 | 1.00 | 19.20 | A | C |
| ATOM | 1823 | CE | LYS | A | 236 | −8.565 | −60.313 | −23.457 | 1.00 | 24.27 | A | C |
| ATOM | 1824 | NZ | LYS | A | 236 | −9.006 | −61.451 | −24.142 | 1.00 | 25.04 | A | N |
| ATOM | 1825 | C | LYS | A | 236 | −12.232 | −61.820 | −20.059 | 1.00 | 17.07 | A | C |
| ATOM | 1826 | O | LYS | A | 236 | −12.539 | −62.351 | −21.166 | 1.00 | 16.49 | A | O |
| ATOM | 1827 | N | ALA | A | 237 | −12.404 | −62.457 | −18.887 | 1.00 | 16.60 | A | N |
| ATOM | 1828 | CA | ALA | A | 237 | −12.982 | −63.842 | −18.917 | 1.00 | 15.77 | A | C |
| ATOM | 1829 | CB | ALA | A | 237 | −12.995 | −64.516 | −17.535 | 1.00 | 13.36 | A | C |
| ATOM | 1830 | C | ALA | A | 237 | −14.408 | −63.738 | −19.500 | 1.00 | 15.82 | A | C |
| ATOM | 1831 | O | ALA | A | 237 | −14.820 | −64.585 | −20.294 | 1.00 | 15.89 | A | O |
| ATOM | 1832 | N | ALA | A | 238 | −15.122 | −62.664 | −19.148 | 1.00 | 14.96 | A | N |
| ATOM | 1833 | CA | ALA | A | 238 | −16.455 | −62.493 | −19.616 | 1.00 | 14.72 | A | C |
| ATOM | 1834 | CB | ALA | A | 238 | −17.103 | −61.346 | −18.843 | 1.00 | 15.43 | A | C |
| ATOM | 1835 | C | ALA | A | 238 | −16.482 | −62.242 | −21.142 | 1.00 | 14.97 | A | C |
| ATOM | 1836 | O | ALA | A | 238 | −17.297 | −62.807 | −21.866 | 1.00 | 15.12 | A | O |
| ATOM | 1837 | N | TYR | A | 239 | −15.574 | −61.427 | −21.633 | 1.00 | 15.89 | A | N |
| ATOM | 1838 | CA | TYR | A | 239 | −15.480 | −61.143 | −23.068 | 1.00 | 17.95 | A | C |
| ATOM | 1839 | CB | TYR | A | 239 | −14.410 | −60.061 | −23.352 | 1.00 | 18.82 | A | C |
| ATOM | 1840 | CG | TYR | A | 239 | −13.741 | −60.115 | −24.716 | 1.00 | 19.94 | A | C |
| ATOM | 1841 | CD1 | TYR | A | 239 | −14.213 | −59.292 | −25.773 | 1.00 | 21.70 | A | C |
| ATOM | 1842 | CE1 | TYR | A | 239 | −13.606 | −59.300 | −27.001 | 1.00 | 19.11 | A | C |
| ATOM | 1843 | CZ | TYR | A | 239 | −12.507 | −60.154 | −27.212 | 1.00 | 19.40 | A | C |
| ATOM | 1844 | OH | TYR | A | 239 | −11.959 | −60.182 | −28.450 | 1.00 | 20.23 | A | O |
| ATOM | 1845 | CE2 | TYR | A | 239 | −12.015 | −60.996 | −26.200 | 1.00 | 16.51 | A | C |
| ATOM | 1846 | CD2 | TYR | A | 239 | −12.640 | −60.970 | −24.960 | 1.00 | 17.39 | A | C |
| ATOM | 1847 | C | TYR | A | 239 | −15.126 | −62.428 | −23.754 | 1.00 | 19.06 | A | C |
| ATOM | 1848 | O | TYR | A | 239 | −15.687 | −62.740 | −24.802 | 1.00 | 21.37 | A | O |
| ATOM | 1849 | N | ASN | A | 240 | −14.229 | −63.216 | −23.175 | 1.00 | 18.84 | A | N |
| ATOM | 1850 | CA | ASN | A | 240 | −13.906 | −64.501 | −23.817 | 1.00 | 17.95 | A | C |
| ATOM | 1851 | CB | ASN | A | 240 | −12.789 | −65.267 | −23.125 | 1.00 | 17.02 | A | C |
| ATOM | 1852 | CG | ASN | A | 240 | −11.459 | −64.609 | −23.259 | 1.00 | 16.24 | A | C |
| ATOM | 1853 | OD1 | ASN | A | 240 | −11.195 | −63.810 | −24.179 | 1.00 | 18.47 | A | O |
| ATOM | 1854 | ND2 | ASN | A | 240 | −10.575 | −64.965 | −22.360 | 1.00 | 13.19 | A | N |
| ATOM | 1855 | C | ASN | A | 240 | −15.126 | −65.358 | −23.841 | 1.00 | 17.29 | A | C |
| ATOM | 1856 | O | ASN | A | 240 | −15.321 | −66.126 | −24.820 | 1.00 | 17.61 | A | O |
| ATOM | 1857 | N | THR | A | 241 | −15.960 | −65.253 | −22.803 | 1.00 | 16.40 | A | N |
| ATOM | 1858 | CA | THR | A | 241 | −17.108 | −66.151 | −22.843 | 1.00 | 16.12 | A | C |
| ATOM | 1859 | CB | THR | A | 241 | −17.653 | −66.698 | −21.463 | 1.00 | 16.69 | A | C |
| ATOM | 1860 | OG1 | THR | A | 241 | −19.095 | −66.600 | −21.334 | 1.00 | 18.84 | A | O |
| ATOM | 1861 | CG2 | THR | A | 241 | −16.875 | −66.395 | −20.334 | 1.00 | 5.55 | A | C |
| ATOM | 1862 | C | THR | A | 241 | −18.214 | −65.732 | −23.801 | 1.00 | 16.89 | A | C |
| ATOM | 1863 | O | THR | A | 241 | −18.763 | −66.561 | −24.531 | 1.00 | 17.17 | A | O |
| ATOM | 1864 | N | ILE | A | 242 | −18.475 | −64.435 | −23.829 | 1.00 | 16.90 | A | N |
| ATOM | 1865 | CA | ILE | A | 242 | −19.404 | −63.861 | −24.758 | 1.00 | 16.79 | A | C |
| ATOM | 1866 | CB | ILE | A | 242 | −19.520 | −62.366 | −24.521 | 1.00 | 17.11 | A | C |
| ATOM | 1867 | CG1 | ILE | A | 242 | −20.083 | −62.103 | −23.129 | 1.00 | 13.60 | A | C |
| ATOM | 1868 | CD1 | ILE | A | 242 | −19.869 | −60.607 | −22.655 | 1.00 | 17.41 | A | C |
| ATOM | 1869 | CG2 | ILE | A | 242 | −20.358 | −61.731 | −25.622 | 1.00 | 13.89 | A | C |
| ATOM | 1870 | C | ILE | A | 242 | −19.070 | −64.125 | −26.232 | 1.00 | 17.41 | A | C |
| ATOM | 1871 | O | ILE | A | 242 | −19.983 | −64.398 | −27.051 | 1.00 | 17.82 | A | O |
| ATOM | 1872 | N | THR | A | 243 | −17.793 | −64.041 | −26.583 | 1.00 | 17.72 | A | N |
| ATOM | 1873 | CA | THR | A | 243 | −17.392 | −64.274 | −27.982 | 1.00 | 18.06 | A | C |
| ATOM | 1874 | CB | THR | A | 243 | −15.931 | −63.774 | −28.295 | 1.00 | 17.72 | A | C |
| ATOM | 1875 | OG1 | THR | A | 243 | −15.019 | −64.485 | −27.481 | 1.00 | 17.90 | A | O |
| ATOM | 1876 | CG2 | THR | A | 243 | −15.758 | −62.235 | −27.989 | 1.00 | 14.22 | A | C |
| ATOM | 1877 | C | THR | A | 243 | −17.501 | −65.747 | −28.366 | 1.00 | 20.21 | A | C |
| ATOM | 1878 | O | THR | A | 243 | −17.439 | −66.072 | −29.545 | 1.00 | 20.40 | A | O |
| ATOM | 1879 | N | LYS | A | 244 | −17.602 | −66.650 | −27.377 | 1.00 | 21.24 | A | N |
| ATOM | 1880 | CA | LYS | A | 244 | −17.624 | −68.092 | −27.650 | 1.00 | 21.87 | A | C |
| ATOM | 1881 | CB | LYS | A | 244 | −16.972 | −68.936 | −26.509 | 1.00 | 21.68 | A | C |
| ATOM | 1882 | CG | LYS | A | 244 | −15.628 | −69.561 | −26.846 | 1.00 | 27.48 | A | C |
| ATOM | 1883 | CD | LYS | A | 244 | −14.538 | −69.622 | −25.605 | 1.00 | 35.73 | A | C |
| ATOM | 1884 | CE | LYS | A | 244 | −13.219 | −70.459 | −25.990 | 1.00 | 38.68 | A | C |
| ATOM | 1885 | NZ | LYS | A | 244 | −13.444 | −72.043 | −26.349 | 1.00 | 33.65 | A | N |
| ATOM | 1886 | C | LYS | A | 244 | −19.073 | −68.462 | −27.796 | 1.00 | 21.76 | A | C |
| ATOM | 1887 | O | LYS | A | 244 | −19.422 | −69.182 | −28.721 | 1.00 | 21.38 | A | O |
| ATOM | 1888 | N | ILE | A | 245 | −19.918 | −67.949 | −26.884 | 1.00 | 21.31 | A | N |
| ATOM | 1889 | CA | ILE | A | 245 | −21.286 | −68.479 | −26.727 | 1.00 | 20.42 | A | C |
| ATOM | 1890 | CB | ILE | A | 245 | −21.520 | −69.132 | −25.334 | 1.00 | 20.57 | A | C |
| ATOM | 1891 | CG1 | ILE | A | 245 | −21.640 | −68.087 | −24.227 | 1.00 | 18.94 | A | C |
| ATOM | 1892 | CD1 | ILE | A | 245 | −22.106 | −68.673 | −22.920 | 1.00 | 17.00 | A | C |
| ATOM | 1893 | CG2 | ILE | A | 245 | −20.419 | −70.121 | −24.984 | 1.00 | 20.88 | A | C |
| ATOM | 1894 | C | ILE | A | 245 | −22.407 | −67.471 | −27.044 | 1.00 | 20.62 | A | C |
| ATOM | 1895 | O | ILE | A | 245 | −23.554 | −67.877 | −27.233 | 1.00 | 21.25 | A | O |
| ATOM | 1896 | N | GLY | A | 246 | −22.076 | −66.182 | −27.160 | 1.00 | 19.44 | A | N |
| ATOM | 1897 | CA | GLY | A | 246 | −23.054 | −65.205 | −27.613 | 1.00 | 20.30 | A | C |
| ATOM | 1898 | C | GLY | A | 246 | −23.672 | −64.532 | −26.400 | 1.00 | 20.27 | A | C |
| ATOM | 1899 | O | GLY | A | 246 | −23.565 | −65.092 | −25.298 | 1.00 | 19.99 | A | O |
| ATOM | 1900 | N | VAL | A | 247 | −24.275 | −63.348 | −26.583 | 1.00 | 19.71 | A | N |
| ATOM | 1901 | CA | VAL | A | 247 | −24.808 | −62.615 | −25.468 | 1.00 | 20.66 | A | C |
| ATOM | 1902 | CB | VAL | A | 247 | −24.838 | −61.037 | −25.578 | 1.00 | 21.06 | A | C |
| ATOM | 1903 | CG1 | VAL | A | 247 | −24.159 | −60.493 | −26.787 | 1.00 | 20.58 | A | C |
| ATOM | 1904 | CG2 | VAL | A | 247 | −26.206 | −60.431 | −25.285 | 1.00 | 21.14 | A | C |
| ATOM | 1905 | C | VAL | A | 247 | −26.030 | −63.180 | −24.828 | 1.00 | 21.73 | A | C |
| ATOM | 1906 | O | VAL | A | 247 | −26.071 | −63.239 | −23.616 | 1.00 | 23.82 | A | O |
| ATOM | 1907 | N | ASN | A | 248 | −26.975 | −63.695 | −25.594 | 1.00 | 21.97 | A | N |
| ATOM | 1908 | CA | ASN | A | 248 | −28.169 | −64.283 | −25.003 | 1.00 | 23.02 | A | C |
| ATOM | 1909 | CB | ASN | A | 248 | −29.082 | −64.962 | −26.087 | 1.00 | 23.95 | A | C |
| ATOM | 1910 | CG | ASN | A | 248 | −29.794 | −63.932 | −27.012 | 1.00 | 29.57 | A | C |
| ATOM | 1911 | OD1 | ASN | A | 248 | −30.030 | −64.209 | −28.217 | 1.00 | 42.08 | A | O |
| ATOM | 1912 | ND2 | ASN | A | 248 | −30.108 | −62.755 | −26.485 | 1.00 | 30.06 | A | N |
| ATOM | 1913 | C | ASN | A | 248 | −27.822 | −65.295 | −23.931 | 1.00 | 21.63 | A | C |
| ATOM | 1914 | O | ASN | A | 248 | −28.454 | −65.298 | −22.887 | 1.00 | 22.73 | A | O |
| ATOM | 1915 | N | LYS | A | 249 | −26.903 | −66.221 | −24.226 | 1.00 | 19.62 | A | N |
| ATOM | 1916 | CA | LYS | A | 249 | −26.557 | −67.246 | −23.272 | 1.00 | 18.79 | A | C |
| ATOM | 1917 | CB | LYS | A | 249 | −25.866 | −68.406 | −23.961 | 1.00 | 18.46 | A | C |
| ATOM | 1918 | CG | LYS | A | 249 | −26.757 | −69.173 | −24.902 | 1.00 | 19.59 | A | C |
| ATOM | 1919 | CD | LYS | A | 249 | −26.019 | −70.451 | −25.276 | 1.00 | 23.34 | A | C |
| ATOM | 1920 | CE | LYS | A | 249 | −26.834 | −71.447 | −26.132 | 1.00 | 23.02 | A | C |
| ATOM | 1921 | NZ | LYS | A | 249 | −25.958 | −72.544 | −26.700 | 1.00 | 24.35 | A | N |
| ATOM | 1922 | C | LYS | A | 249 | −25.692 | −66.650 | −22.150 | 1.00 | 18.27 | A | C |
| ATOM | 1923 | O | LYS | A | 249 | −25.931 | −66.895 | −20.964 | 1.00 | 18.67 | A | O |
| ATOM | 1924 | N | ALA | A | 250 | −24.728 | −65.812 | −22.511 | 1.00 | 18.25 | A | N |
| ATOM | 1925 | CA | ALA | A | 250 | −23.781 | −65.288 | −21.533 | 1.00 | 18.63 | A | C |
| ATOM | 1926 | CB | ALA | A | 250 | −22.612 | −64.563 | −22.178 | 1.00 | 16.62 | A | C |
| ATOM | 1927 | C | ALA | A | 250 | −24.490 | −64.403 | −20.508 | 1.00 | 19.22 | A | C |
| ATOM | 1928 | O | ALA | A | 250 | −24.128 | −64.419 | −19.319 | 1.00 | 19.71 | A | O |
| ATOM | 1929 | N | GLU | A | 251 | −25.513 | −63.667 | −20.930 | 1.00 | 19.16 | A | N |
| ATOM | 1930 | CA | GLU | A | 251 | −26.133 | −62.772 | −19.962 | 1.00 | 18.90 | A | C |
| ATOM | 1931 | CB | GLU | A | 251 | −26.864 | −61.637 | −20.610 | 1.00 | 17.73 | A | C |
| ATOM | 1932 | CG | GLU | A | 251 | −28.130 | −62.056 | −21.261 | 1.00 | 19.93 | A | C |
| ATOM | 1933 | CD | GLU | A | 251 | −28.718 | −60.975 | −22.197 | 1.00 | 20.89 | A | C |
| ATOM | 1934 | OE1 | GLU | A | 251 | −28.226 | −59.813 | −22.204 | 1.00 | 16.53 | A | O |
| ATOM | 1935 | OE2 | GLU | A | 251 | −29.671 | −61.319 | −22.925 | 1.00 | 22.41 | A | O |
| ATOM | 1936 | C | GLU | A | 251 | −27.018 | −63.599 | −18.987 | 1.00 | 19.67 | A | C |
| ATOM | 1937 | O | GLU | A | 251 | −27.126 | −63.256 | −17.811 | 1.00 | 18.68 | A | O |
| ATOM | 1938 | N | GLN | A | 252 | −27.591 | −64.720 | −19.450 | 1.00 | 18.71 | A | N |
| ATOM | 1939 | CA | GLN | A | 252 | −28.298 | −65.580 | −18.502 | 1.00 | 17.84 | A | C |
| ATOM | 1940 | CB | GLN | A | 252 | −29.096 | −66.642 | −19.250 | 1.00 | 17.39 | A | C |
| ATOM | 1941 | CG | GLN | A | 252 | −30.141 | −66.048 | −20.183 | 1.00 | 17.73 | A | C |
| ATOM | 1942 | CD | GLN | A | 252 | −31.265 | −65.373 | −19.427 | 1.00 | 23.25 | A | C |
| ATOM | 1943 | OE1 | GLN | A | 252 | −31.531 | −65.695 | −18.253 | 1.00 | 24.70 | A | O |
| ATOM | 1944 | NE2 | GLN | A | 252 | −31.902 | −64.382 | −20.068 | 1.00 | 21.47 | A | N |
| ATOM | 1945 | C | GLN | A | 252 | −27.316 | −66.222 | −17.496 | 1.00 | 18.12 | A | C |
| ATOM | 1946 | O | GLN | A | 252 | −27.576 | −66.284 | −16.292 | 1.00 | 18.70 | A | O |
| ATOM | 1947 | N | ILE | A | 253 | −26.167 | −66.661 | −17.983 | 1.00 | 17.81 | A | N |
| ATOM | 1948 | CA | ILE | A | 253 | −25.181 | −67.356 | −17.153 | 1.00 | 16.02 | A | C |
| ATOM | 1949 | CB | ILE | A | 253 | −24.079 | −67.899 | −18.093 | 1.00 | 15.55 | A | C |
| ATOM | 1950 | CG1 | ILE | A | 253 | −24.629 | −69.145 | −18.832 | 1.00 | 15.24 | A | C |
| ATOM | 1951 | CD1 | ILE | A | 253 | −23.758 | −69.590 | −20.013 | 1.00 | 15.45 | A | C |
| ATOM | 1952 | CG2 | ILE | A | 253 | −22.759 | −68.151 | −17.353 | 1.00 | 11.68 | A | C |
| ATOM | 1953 | C | ILE | A | 253 | −24.659 | −66.419 | −16.024 | 1.00 | 16.62 | A | C |
| ATOM | 1954 | O | ILE | A | 253 | −24.695 | −66.802 | −14.828 | 1.00 | 15.86 | A | O |
| ATOM | 1955 | N | TYR | A | 254 | −24.283 | −65.187 | −16.423 | 1.00 | 16.13 | A | N |
| ATOM | 1956 | CA | TYR | A | 254 | −23.859 | −64.098 | −15.561 | 1.00 | 16.69 | A | C |
| ATOM | 1957 | CB | TYR | A | 254 | −23.426 | −62.842 | −16.387 | 1.00 | 15.61 | A | C |
| ATOM | 1958 | CG | TYR | A | 254 | −21.977 | −62.980 | −16.659 | 1.00 | 16.81 | A | C |
| ATOM | 1959 | CD1 | TYR | A | 254 | −21.025 | −62.441 | −15.767 | 1.00 | 15.95 | A | C |
| ATOM | 1960 | CE1 | TYR | A | 254 | −19.660 | −62.726 | −15.920 | 1.00 | 16.12 | A | C |
| ATOM | 1961 | CZ | TYR | A | 254 | −19.265 | −63.540 | −16.983 | 1.00 | 16.40 | A | C |
| ATOM | 1962 | OH | TYR | A | 254 | −17.933 | −63.797 | −17.199 | 1.00 | 15.02 | A | O |
| ATOM | 1963 | CE2 | TYR | A | 254 | −20.194 | −64.080 | −17.871 | 1.00 | 15.41 | A | C |
| ATOM | 1964 | CD2 | TYR | A | 254 | −21.529 | −63.828 | −17.700 | 1.00 | 13.73 | A | C |
| ATOM | 1965 | C | TYR | A | 254 | −24.888 | −63.742 | −14.479 | 1.00 | 18.24 | A | C |
| ATOM | 1966 | O | TYR | A | 254 | −24.514 | −63.497 | −13.318 | 1.00 | 18.18 | A | O |
| ATOM | 1967 | N | TYR | A | 255 | −26.160 | −63.727 | −14.863 | 1.00 | 18.34 | A | N |
| ATOM | 1968 | CA | TYR | A | 255 | −27.226 | −63.290 | −13.994 | 1.00 | 18.95 | A | C |
| ATOM | 1969 | CB | TYR | A | 255 | −28.510 | −62.998 | −14.805 | 1.00 | 17.25 | A | C |
| ATOM | 1970 | CG | TYR | A | 255 | −29.636 | −62.557 | −13.921 | 1.00 | 16.18 | A | C |
| ATOM | 1971 | CD1 | TYR | A | 255 | −29.658 | −61.279 | −13.396 | 1.00 | 14.41 | A | C |
| ATOM | 1972 | CE1 | TYR | A | 255 | −30.653 | −60.865 | −12.553 | 1.00 | 17.82 | A | C |
| ATOM | 1973 | CZ | TYR | A | 255 | −31.640 | −61.758 | −12.173 | 1.00 | 19.38 | A | C |
| ATOM | 1974 | OH | TYR | A | 255 | −32.618 | −61.323 | −11.341 | 1.00 | 20.14 | A | O |
| ATOM | 1975 | CE2 | TYR | A | 255 | −31.642 | −63.055 | −12.644 | 1.00 | 18.82 | A | C |
| ATOM | 1976 | CD2 | TYR | A | 255 | −30.620 | −63.447 | −13.526 | 1.00 | 17.58 | A | C |
| ATOM | 1977 | C | TYR | A | 255 | −27.441 | −64.415 | −12.940 | 1.00 | 20.84 | A | C |
| ATOM | 1978 | O | TYR | A | 255 | −27.610 | −64.166 | −11.724 | 1.00 | 20.60 | A | O |
| ATOM | 1979 | N | ARG | A | 256 | −27.438 | −65.652 | −13.423 | 1.00 | 21.16 | A | N |
| ATOM | 1980 | CA | ARG | A | 256 | −27.617 | −66.775 | −12.554 | 1.00 | 21.24 | A | C |
| ATOM | 1981 | CB | ARG | A | 256 | −27.617 | −68.057 | −13.402 | 1.00 | 20.10 | A | C |
| ATOM | 1982 | CG | ARG | A | 256 | −27.889 | −69.310 | −12.614 | 1.00 | 20.46 | A | C |
| ATOM | 1983 | CD | ARG | A | 256 | −28.636 | −70.402 | −13.386 | 1.00 | 22.52 | A | C |
| ATOM | 1984 | NE | ARG | A | 256 | −29.042 | −71.451 | −12.449 | 1.00 | 25.65 | A | N |
| ATOM | 1985 | CZ | ARG | A | 256 | −30.190 | −71.448 | −11.756 | 1.00 | 24.08 | A | C |
| ATOM | 1986 | NH1 | ARG | A | 256 | −31.107 | −70.479 | −11.918 | 1.00 | 18.40 | A | N |
| ATOM | 1987 | NH2 | ARG | A | 256 | −30.395 | −72.414 | −10.890 | 1.00 | 18.20 | A | N |
| ATOM | 1988 | C | ARG | A | 256 | −26.455 | −66.748 | −11.539 | 1.00 | 21.58 | A | C |
| ATOM | 1989 | O | ARG | A | 256 | −26.664 | −66.813 | −10.345 | 1.00 | 23.44 | A | O |
| ATOM | 1990 | N | ALA | A | 257 | −25.226 | −66.616 | −12.003 | 1.00 | 21.29 | A | N |
| ATOM | 1991 | CA | ALA | A | 257 | −24.070 | −66.707 | −11.091 | 1.00 | 20.37 | A | C |
| ATOM | 1992 | CB | ALA | A | 257 | −22.807 | −66.514 | −11.846 | 1.00 | 18.20 | A | C |
| ATOM | 1993 | C | ALA | A | 257 | −24.154 | −65.660 | −9.994 | 1.00 | 20.56 | A | C |
| ATOM | 1994 | O | ALA | A | 257 | −23.861 | −65.947 | −8.809 | 1.00 | 20.92 | A | O |
| ATOM | 1995 | N | LEU | A | 258 | −24.536 | −64.448 | −10.406 | 1.00 | 20.17 | A | N |
| ATOM | 1996 | CA | LEU | A | 258 | −24.565 | −63.313 | −9.543 | 1.00 | 20.36 | A | C |
| ATOM | 1997 | CB | LEU | A | 258 | −24.787 | −62.029 | −10.315 | 1.00 | 19.99 | A | C |
| ATOM | 1998 | CG | LEU | A | 258 | −24.858 | −60.759 | −9.436 | 1.00 | 20.59 | A | C |
| ATOM | 1999 | CD1 | LEU | A | 258 | −23.600 | −60.617 | −8.588 | 1.00 | 16.80 | A | C |
| ATOM | 2000 | CD2 | LEU | A | 258 | −25.098 | −59.441 | −10.256 | 1.00 | 18.81 | A | C |
| ATOM | 2001 | C | LEU | A | 258 | −25.643 | −63.493 | −8.471 | 1.00 | 21.49 | A | C |
| ATOM | 2002 | O | LEU | A | 258 | −25.359 | −63.253 | −7.290 | 1.00 | 20.13 | A | O |
| ATOM | 2003 | N | THR | A | 259 | −26.814 | −64.018 | −8.861 | 1.00 | 21.25 | A | N |
| ATOM | 2004 | CA | THR | A | 259 | −27.954 | −63.957 | −8.010 | 1.00 | 22.14 | A | C |
| ATOM | 2005 | CB | THR | A | 259 | −29.256 | −63.572 | −8.813 | 1.00 | 22.89 | A | C |
| ATOM | 2006 | OG1 | THR | A | 259 | −29.521 | −64.589 | −9.777 | 1.00 | 21.97 | A | O |
| ATOM | 2007 | CG2 | THR | A | 259 | −29.109 | −62.251 | −9.512 | 1.00 | 21.29 | A | C |
| ATOM | 2008 | C | THR | A | 259 | −28.188 | −65.287 | −7.337 | 1.00 | 23.10 | A | C |
| ATOM | 2009 | O | THR | A | 259 | −29.019 | −65.396 | −6.446 | 1.00 | 22.98 | A | O |
| ATOM | 2010 | N | VAL | A | 260 | −27.497 | −66.334 | −7.749 | 1.00 | 23.70 | A | N |
| ATOM | 2011 | CA | VAL | A | 260 | −27.751 | −67.645 | −7.104 | 1.00 | 22.78 | A | C |
| ATOM | 2012 | CB | VAL | A | 260 | −28.299 | −68.684 | −8.114 | 1.00 | 22.81 | A | C |
| ATOM | 2013 | CG1 | VAL | A | 260 | −28.313 | −70.064 | −7.524 | 1.00 | 21.92 | A | C |
| ATOM | 2014 | CG2 | VAL | A | 260 | −29.712 | −68.253 | −8.612 | 1.00 | 22.11 | A | C |
| ATOM | 2015 | C | VAL | A | 260 | −26.561 | −68.217 | −6.345 | 1.00 | 22.91 | A | C |
| ATOM | 2016 | O | VAL | A | 260 | −26.741 | −68.754 | −5.252 | 1.00 | 23.99 | A | O |
| ATOM | 2017 | N | TYR | A | 261 | −25.366 | −68.069 | −6.897 | 1.00 | 21.81 | A | N |
| ATOM | 2018 | CA | TYR | A | 261 | −24.181 | −68.768 | −6.417 | 1.00 | 21.43 | A | C |
| ATOM | 2019 | CB | TYR | A | 261 | −23.470 | −69.511 | −7.565 | 1.00 | 20.42 | A | C |
| ATOM | 2020 | CG | TYR | A | 261 | −24.256 | −70.683 | −8.061 | 1.00 | 19.92 | A | C |
| ATOM | 2021 | CD1 | TYR | A | 261 | −24.485 | −71.807 | −7.226 | 1.00 | 20.08 | A | C |
| ATOM | 2022 | CE1 | TYR | A | 261 | −25.248 | −72.907 | −7.649 | 1.00 | 19.11 | A | C |
| ATOM | 2023 | CZ | TYR | A | 261 | −25.785 | −72.870 | −8.926 | 1.00 | 23.20 | A | C |
| ATOM | 2024 | OH | TYR | A | 261 | −26.510 | −73.952 | −9.382 | 1.00 | 21.60 | A | O |
| ATOM | 2025 | CE2 | TYR | A | 261 | −25.539 | −71.750 | −9.788 | 1.00 | 18.76 | A | C |
| ATOM | 2026 | CD2 | TYR | A | 261 | −24.775 | −70.695 | −9.342 | 1.00 | 17.58 | A | C |
| ATOM | 2027 | C | TYR | A | 261 | −23.165 | −67.922 | −5.704 | 1.00 | 21.41 | A | C |
| ATOM | 2028 | O | TYR | A | 261 | −22.458 | −68.423 | −4.888 | 1.00 | 23.70 | A | O |
| ATOM | 2029 | N | LEU | A | 262 | −23.052 | −66.646 | −6.011 | 1.00 | 21.84 | A | N |
| ATOM | 2030 | CA | LEU | A | 262 | −21.933 | −65.876 | −5.498 | 1.00 | 20.81 | A | C |
| ATOM | 2031 | CB | LEU | A | 262 | −21.499 | −64.783 | −6.525 | 1.00 | 20.64 | A | C |
| ATOM | 2032 | CG | LEU | A | 262 | −20.712 | −65.319 | −7.777 | 1.00 | 17.72 | A | C |
| ATOM | 2033 | CD1 | LEU | A | 262 | −20.347 | −64.218 | −8.742 | 1.00 | 15.44 | A | C |
| ATOM | 2034 | CD2 | LEU | A | 262 | −19.485 | −66.126 | −7.488 | 1.00 | 13.13 | A | C |
| ATOM | 2035 | C | LEU | A | 262 | −22.211 | −65.361 | −4.075 | 1.00 | 21.67 | A | C |
| ATOM | 2036 | O | LEU | A | 262 | −23.357 | −65.179 | −3.681 | 1.00 | 21.61 | A | O |
| ATOM | 2037 | N | THR | A | 263 | −21.153 | −65.179 | −3.288 | 1.00 | 22.08 | A | N |
| ATOM | 2038 | CA | THR | A | 263 | −21.296 | −64.852 | −1.855 | 1.00 | 22.85 | A | C |
| ATOM | 2039 | CB | THR | A | 263 | −20.821 | −66.011 | −0.975 | 1.00 | 22.90 | A | C |
| ATOM | 2040 | OG1 | THR | A | 263 | −19.435 | −66.202 | −1.246 | 1.00 | 24.49 | A | O |
| ATOM | 2041 | CG2 | THR | A | 263 | −21.591 | −67.324 | −1.303 | 1.00 | 20.75 | A | C |
| ATOM | 2042 | C | THR | A | 263 | −20.430 | −63.596 | −1.581 | 1.00 | 23.07 | A | C |
| ATOM | 2043 | O | THR | A | 263 | −19.704 | −63.163 | −2.461 | 1.00 | 23.16 | A | O |
| ATOM | 2044 | N | PRO | A | 264 | −20.524 | −62.984 | −0.381 | 1.00 | 22.93 | A | N |
| ATOM | 2045 | CA | PRO | A | 264 | −19.713 | −61.734 | −0.268 | 1.00 | 21.80 | A | C |
| ATOM | 2046 | CB | PRO | A | 264 | −20.013 | −61.259 | 1.168 | 1.00 | 20.85 | A | C |
| ATOM | 2047 | CG | PRO | A | 264 | −21.494 | −61.677 | 1.307 | 1.00 | 20.96 | A | C |
| ATOM | 2048 | CD | PRO | A | 264 | −21.548 | −63.073 | 0.686 | 1.00 | 21.37 | A | C |
| ATOM | 2049 | C | PRO | A | 264 | −18.219 | −61.927 | −0.449 | 1.00 | 21.95 | A | C |
| ATOM | 2050 | O | PRO | A | 264 | −17.527 | −60.980 | −0.854 | 1.00 | 21.74 | A | O |
| ATOM | 2051 | N | SER | A | 265 | −17.689 | −63.107 | −0.155 | 1.00 | 21.51 | A | N |
| ATOM | 2052 | CA | SER | A | 265 | −16.245 | −63.202 | −0.203 | 1.00 | 22.77 | A | C |
| ATOM | 2053 | CB | SER | A | 265 | −15.708 | −63.723 | 1.131 | 1.00 | 23.22 | A | C |
| ATOM | 2054 | OG | SER | A | 265 | −16.294 | −64.984 | 1.338 | 1.00 | 27.76 | A | O |
| ATOM | 2055 | C | SER | A | 265 | −15.714 | −64.054 | −1.385 | 1.00 | 22.08 | A | C |
| ATOM | 2056 | O | SER | A | 265 | −14.572 | −64.565 | −1.337 | 1.00 | 21.37 | A | O |
| ATOM | 2057 | N | SER | A | 266 | −16.554 | −64.203 | −2.402 | 1.00 | 21.31 | A | N |
| ATOM | 2058 | CA | SER | A | 266 | −16.239 | −64.941 | −3.615 | 1.00 | 21.74 | A | C |
| ATOM | 2059 | CB | SER | A | 266 | −17.390 | −64.849 | −4.624 | 1.00 | 20.96 | A | C |
| ATOM | 2060 | OG | SER | A | 266 | −18.465 | −65.654 | −4.203 | 1.00 | 23.84 | A | O |
| ATOM | 2061 | C | SER | A | 266 | −14.949 | −64.428 | −4.260 | 1.00 | 21.37 | A | C |
| ATOM | 2062 | O | SER | A | 266 | −14.822 | −63.246 | −4.543 | 1.00 | 21.41 | A | O |
| ATOM | 2063 | N | THR | A | 267 | −13.993 | −65.334 | −4.447 | 1.00 | 19.86 | A | N |
| ATOM | 2064 | CA | THR | A | 267 | −12.823 | −65.083 | −5.229 | 1.00 | 18.51 | A | C |
| ATOM | 2065 | CB | THR | A | 267 | −11.802 | −66.126 | −4.784 | 1.00 | 19.66 | A | C |
| ATOM | 2066 | OG1 | THR | A | 267 | −12.308 | −67.447 | −5.103 | 1.00 | 19.50 | A | O |
| ATOM | 2067 | CG2 | THR | A | 267 | −11.539 | −66.029 | −3.234 | 1.00 | 17.09 | A | C |
| ATOM | 2068 | C | THR | A | 267 | −13.116 | −65.295 | −6.759 | 1.00 | 19.26 | A | C |
| ATOM | 2069 | O | THR | A | 267 | −14.219 | −65.810 | −7.179 | 1.00 | 18.49 | A | O |
| ATOM | 2070 | N | PHE | A | 268 | −12.140 | −64.915 | −7.597 | 1.00 | 18.40 | A | N |
| ATOM | 2071 | CA | PHE | A | 268 | −12.115 | −65.257 | −9.035 | 1.00 | 17.62 | A | C |
| ATOM | 2072 | CB | PHE | A | 268 | −10.766 | −64.858 | −9.626 | 1.00 | 17.69 | A | C |
| ATOM | 2073 | CG | PHE | A | 268 | −10.559 | −63.372 | −9.729 | 1.00 | 15.59 | A | C |
| ATOM | 2074 | CD1 | PHE | A | 268 | −11.476 | −62.467 | −9.151 | 1.00 | 15.00 | A | C |
| ATOM | 2075 | CE1 | PHE | A | 268 | −11.320 | −61.031 | −9.284 | 1.00 | 14.29 | A | C |
| ATOM | 2076 | CZ | PHE | A | 268 | −10.192 | −60.528 | −9.924 | 1.00 | 10.88 | A | C |
| ATOM | 2077 | CE2 | PHE | A | 268 | −9.224 | −61.438 | −10.418 | 1.00 | 16.00 | A | C |
| ATOM | 2078 | CD2 | PHE | A | 268 | −9.422 | −62.870 | −10.339 | 1.00 | 12.08 | A | C |
| ATOM | 2079 | C | PHE | A | 268 | −12.310 | −66.753 | −9.254 | 1.00 | 18.07 | A | C |
| ATOM | 2080 | O | PHE | A | 268 | −13.080 | −67.204 | −10.110 | 1.00 | 18.45 | A | O |
| ATOM | 2081 | N | LYS | A | 269 | −11.604 | −67.547 | −8.493 | 1.00 | 18.34 | A | N |
| ATOM | 2082 | CA | LYS | A | 269 | −11.833 | −68.966 | −8.606 | 1.00 | 20.19 | A | C |
| ATOM | 2083 | CB | LYS | A | 269 | −10.924 | −69.711 | −7.635 | 1.00 | 20.91 | A | C |
| ATOM | 2084 | CG | LYS | A | 269 | −10.228 | −70.808 | −8.311 | 1.00 | 24.31 | A | C |
| ATOM | 2085 | CD | LYS | A | 269 | −8.941 | −70.383 | −8.907 | 1.00 | 23.96 | A | C |
| ATOM | 2086 | CE | LYS | A | 269 | −8.774 | −71.095 | −10.196 | 1.00 | 24.14 | A | C |
| ATOM | 2087 | NZ | LYS | A | 269 | −7.867 | −72.214 | −10.403 | 1.00 | 20.27 | A | N |
| ATOM | 2088 | C | LYS | A | 269 | −13.302 | −69.340 | −8.328 | 1.00 | 20.16 | A | C |
| ATOM | 2089 | O | LYS | A | 269 | −13.864 | −70.209 | −9.017 | 1.00 | 19.03 | A | O |
| ATOM | 2090 | N | ASP | A | 270 | −13.942 | −68.678 | −7.342 | 1.00 | 20.01 | A | N |
| ATOM | 2091 | CA | ASP | A | 270 | −15.339 | −69.019 | −7.061 | 1.00 | 19.27 | A | C |
| ATOM | 2092 | CB | ASP | A | 270 | −15.851 | −68.360 | −5.764 | 1.00 | 18.81 | A | C |
| ATOM | 2093 | CG | ASP | A | 270 | −15.108 | −68.810 | −4.541 | 1.00 | 21.89 | A | C |
| ATOM | 2094 | OD1 | ASP | A | 270 | −14.949 | −70.010 | −4.345 | 1.00 | 23.08 | A | O |
| ATOM | 2095 | OD2 | ASP | A | 270 | −14.667 | −67.973 | −3.738 | 1.00 | 25.68 | A | O |
| ATOM | 2096 | C | ASP | A | 270 | −16.226 | −68.573 | −8.236 | 1.00 | 18.97 | A | C |
| ATOM | 2097 | O | ASP | A | 270 | −17.210 | −69.268 | −8.563 | 1.00 | 20.23 | A | O |
| ATOM | 2098 | N | ALA | A | 271 | −15.938 | −67.397 | −8.818 | 1.00 | 17.67 | A | N |
| ATOM | 2099 | CA | ALA | A | 271 | −16.789 | −66.827 | −9.864 | 1.00 | 17.60 | A | C |
| ATOM | 2100 | CB | ALA | A | 271 | −16.341 | −65.408 | −10.223 | 1.00 | 17.75 | A | C |
| ATOM | 2101 | C | ALA | A | 271 | −16.719 | −67.744 | −11.122 | 1.00 | 18.15 | A | C |
| ATOM | 2102 | O | ALA | A | 271 | −17.742 | −68.120 | −11.703 | 1.00 | 15.72 | A | O |
| ATOM | 2103 | N | LYS | A | 272 | −15.491 | −68.135 | −11.474 | 1.00 | 17.71 | A | N |
| ATOM | 2104 | CA | LYS | A | 272 | −15.259 | −69.255 | −12.392 | 1.00 | 18.86 | A | C |
| ATOM | 2105 | CB | LYS | A | 272 | −13.796 | −69.746 | −12.312 | 1.00 | 18.42 | A | C |
| ATOM | 2106 | CG | LYS | A | 272 | −13.494 | −70.766 | −13.309 | 1.00 | 17.22 | A | C |
| ATOM | 2107 | CD | LYS | A | 272 | −12.161 | −71.342 | −13.061 | 1.00 | 15.91 | A | C |
| ATOM | 2108 | CE | LYS | A | 272 | −11.739 | −72.203 | −14.227 | 1.00 | 14.80 | A | C |
| ATOM | 2109 | NZ | LYS | A | 272 | −10.791 | −73.326 | −13.827 | 1.00 | 15.95 | A | N |
| ATOM | 2110 | C | LYS | A | 272 | −16.188 | −70.425 | −12.144 | 1.00 | 18.82 | A | C |
| ATOM | 2111 | O | LYS | A | 272 | −16.921 | −70.829 | −13.044 | 1.00 | 20.76 | A | O |
| ATOM | 2112 | N | ALA | A | 273 | −16.160 | −70.970 | −10.941 | 1.00 | 18.23 | A | N |
| ATOM | 2113 | CA | ALA | A | 273 | −16.998 | −72.154 | −10.623 | 1.00 | 19.13 | A | C |
| ATOM | 2114 | CB | ALA | A | 273 | −16.647 | −72.807 | −9.192 | 1.00 | 16.62 | A | C |
| ATOM | 2115 | C | ALA | A | 273 | −18.471 | −71.778 | −10.703 | 1.00 | 19.05 | A | C |
| ATOM | 2116 | O | ALA | A | 273 | −19.307 | −72.577 | −11.169 | 1.00 | 20.74 | A | O |
| ATOM | 2117 | N | ALA | A | 274 | −18.813 | −70.577 | −10.278 | 1.00 | 17.40 | A | N |
| ATOM | 2118 | CA | ALA | A | 274 | −20.247 | −70.246 | −10.346 | 1.00 | 17.80 | A | C |
| ATOM | 2119 | CB | ALA | A | 274 | −20.571 | −69.077 | −9.474 | 1.00 | 15.47 | A | C |
| ATOM | 2120 | C | ALA | A | 274 | −20.745 | −70.018 | −11.791 | 1.00 | 17.78 | A | C |
| ATOM | 2121 | O | ALA | A | 274 | −21.911 | −70.319 | −12.089 | 1.00 | 19.25 | A | O |
| ATOM | 2122 | N | LEU | A | 275 | −19.896 | −69.473 | −12.671 | 1.00 | 16.84 | A | N |
| ATOM | 2123 | CA | LEU | A | 275 | −20.292 | −69.266 | −14.053 | 1.00 | 17.21 | A | C |
| ATOM | 2124 | CB | LEU | A | 275 | −19.396 | −68.234 | −14.730 | 1.00 | 16.62 | A | C |
| ATOM | 2125 | CG | LEU | A | 275 | −19.504 | −66.851 | −14.062 | 1.00 | 16.84 | A | C |
| ATOM | 2126 | CD1 | LEU | A | 275 | −18.140 | −66.091 | −14.152 | 1.00 | 8.70 | A | C |
| ATOM | 2127 | CD2 | LEU | A | 275 | −20.648 | −66.083 | −14.679 | 1.00 | 14.58 | A | C |
| ATOM | 2128 | C | LEU | A | 275 | −20.316 | −70.642 | −14.817 | 1.00 | 17.79 | A | C |
| ATOM | 2129 | O | LEU | A | 275 | −21.207 | −70.909 | −15.632 | 1.00 | 16.56 | A | O |
| ATOM | 2130 | N | ILE | A | 276 | −19.383 | −71.542 | −14.501 | 1.00 | 17.59 | A | N |
| ATOM | 2131 | CA | ILE | A | 276 | −19.467 | −72.859 | −15.076 | 1.00 | 17.24 | A | C |
| ATOM | 2132 | CB | ILE | A | 276 | −18.225 | −73.656 | −14.721 | 1.00 | 17.48 | A | C |
| ATOM | 2133 | CG1 | ILE | A | 276 | −17.017 | −73.094 | −15.440 | 1.00 | 13.52 | A | C |
| ATOM | 2134 | CD1 | ILE | A | 276 | −15.754 | −73.748 | −14.901 | 1.00 | 3.30 | A | C |
| ATOM | 2135 | CG2 | ILE | A | 276 | −18.402 | −75.137 | −15.034 | 1.00 | 17.60 | A | C |
| ATOM | 2136 | C | ILE | A | 276 | −20.749 | −73.565 | −14.630 | 1.00 | 18.56 | A | C |
| ATOM | 2137 | O | ILE | A | 276 | −21.492 | −74.148 | −15.449 | 1.00 | 19.97 | A | O |
| ATOM | 2138 | N | GLN | A | 277 | −21.057 | −73.506 | −13.341 | 1.00 | 18.86 | A | N |
| ATOM | 2139 | CA | GLN | A | 277 | −22.182 | −74.275 | −12.870 | 1.00 | 18.92 | A | C |
| ATOM | 2140 | CB | GLN | A | 277 | −22.273 | −74.263 | −11.334 | 1.00 | 19.61 | A | C |
| ATOM | 2141 | CG | GLN | A | 277 | −23.503 | −74.991 | −10.746 | 1.00 | 21.40 | A | C |
| ATOM | 2142 | CD | GLN | A | 277 | −23.470 | −76.498 | −11.075 | 1.00 | 23.90 | A | C |
| ATOM | 2143 | OE1 | GLN | A | 277 | −22.447 | −77.138 | −10.868 | 1.00 | 25.80 | A | O |
| ATOM | 2144 | NE2 | GLN | A | 277 | −24.577 | −77.045 | −11.614 | 1.00 | 18.38 | A | N |
| ATOM | 2145 | C | GLN | A | 277 | −23.427 | −73.656 | −13.481 | 1.00 | 19.42 | A | C |
| ATOM | 2146 | O | GLN | A | 277 | −24.344 | −74.397 | −13.839 | 1.00 | 21.09 | A | O |
| ATOM | 2147 | N | SER | A | 278 | −23.488 | −72.323 | −13.581 | 1.00 | 18.34 | A | N |
| ATOM | 2148 | CA | SER | A | 278 | −24.692 | −71.660 | −14.126 | 1.00 | 18.25 | A | C |
| ATOM | 2149 | CB | SER | A | 278 | −24.629 | −70.154 | −13.977 | 1.00 | 17.87 | A | C |
| ATOM | 2150 | OG | SER | A | 278 | −24.503 | −69.758 | −12.604 | 1.00 | 16.50 | A | O |
| ATOM | 2151 | C | SER | A | 278 | −24.939 | −72.068 | −15.578 | 1.00 | 19.54 | A | C |
| ATOM | 2152 | O | SER | A | 278 | −26.084 | −72.350 | −15.980 | 1.00 | 20.47 | A | O |
| ATOM | 2153 | N | ALA | A | 279 | −23.860 | −72.206 | −16.337 | 1.00 | 19.79 | A | N |
| ATOM | 2154 | CA | ALA | A | 279 | −23.960 | −72.760 | −17.666 | 1.00 | 21.00 | A | C |
| ATOM | 2155 | CB | ALA | A | 279 | −22.615 | −72.646 | −18.398 | 1.00 | 20.07 | A | C |
| ATOM | 2156 | C | ALA | A | 279 | −24.516 | −74.211 | −17.734 | 1.00 | 21.50 | A | C |
| ATOM | 2157 | O | ALA | A | 279 | −25.345 | −74.508 | −18.588 | 1.00 | 21.44 | A | O |
| ATOM | 2158 | N | ARG | A | 280 | −24.019 | −75.117 | −16.894 | 1.00 | 22.65 | A | N |
| ATOM | 2159 | CA | ARG | A | 280 | −24.598 | −76.453 | −16.826 | 1.00 | 23.31 | A | C |
| ATOM | 2160 | CB | ARG | A | 280 | −23.966 | −77.278 | −15.740 | 1.00 | 23.89 | A | C |
| ATOM | 2161 | CG | ARG | A | 280 | −22.545 | −77.418 | −15.885 | 1.00 | 26.68 | A | C |
| ATOM | 2162 | CD | ARG | A | 280 | −22.073 | −78.462 | −14.928 | 1.00 | 35.43 | A | C |
| ATOM | 2163 | NE | ARG | A | 280 | −21.148 | −79.273 | −15.687 | 1.00 | 44.20 | A | N |
| ATOM | 2164 | CZ | ARG | A | 280 | −19.848 | −79.206 | −15.520 | 1.00 | 46.31 | A | C |
| ATOM | 2165 | NH1 | ARG | A | 280 | −19.351 | −78.425 | −14.558 | 1.00 | 44.34 | A | N |
| ATOM | 2166 | NH2 | ARG | A | 280 | −19.073 | −79.932 | −16.307 | 1.00 | 50.17 | A | N |
| ATOM | 2167 | C | ARG | A | 280 | −26.050 | −76.405 | −16.479 | 1.00 | 23.26 | A | C |
| ATOM | 2168 | O | ARG | A | 280 | −26.803 | −77.133 | −17.062 | 1.00 | 23.68 | A | O |
| ATOM | 2169 | N | ASP | A | 281 | −26.446 | −75.567 | −15.519 | 1.00 | 23.26 | A | N |
| ATOM | 2170 | CA | ASP | A | 281 | −27.850 | −75.536 | −15.101 | 1.00 | 23.49 | A | C |
| ATOM | 2171 | CB | ASP | A | 281 | −28.145 | −74.472 | −14.012 | 1.00 | 24.21 | A | C |
| ATOM | 2172 | CG | ASP | A | 281 | −27.444 | −74.729 | −12.668 | 1.00 | 26.18 | A | C |
| ATOM | 2173 | OD1 | ASP | A | 281 | −26.972 | −75.873 | −12.401 | 1.00 | 24.91 | A | O |
| ATOM | 2174 | OD2 | ASP | A | 281 | −27.406 | −73.738 | −11.864 | 1.00 | 26.85 | A | O |
| ATOM | 2175 | C | ASP | A | 281 | −28.701 | −75.177 | −16.315 | 1.00 | 22.86 | A | C |
| ATOM | 2176 | O | ASP | A | 281 | −29.726 | −75.776 | −16.535 | 1.00 | 21.44 | A | O |
| ATOM | 2177 | N | LEU | A | 282 | −28.284 | −74.155 | −17.063 | 1.00 | 22.26 | A | N |
| ATOM | 2178 | CA | LEU | A | 282 | −29.152 | −73.538 | −18.067 | 1.00 | 22.50 | A | C |
| ATOM | 2179 | CB | LEU | A | 282 | −28.882 | −72.041 | −18.151 | 1.00 | 21.78 | A | C |
| ATOM | 2180 | CG | LEU | A | 282 | −29.252 | −71.174 | −16.961 | 1.00 | 22.74 | A | C |
| ATOM | 2181 | CD1 | LEU | A | 282 | −28.603 | −69.829 | −17.165 | 1.00 | 19.04 | A | C |
| ATOM | 2182 | CD2 | LEU | A | 282 | −30.773 | −71.069 | −16.821 | 1.00 | 20.56 | A | C |
| ATOM | 2183 | C | LEU | A | 282 | −29.018 | −74.180 | −19.462 | 1.00 | 22.63 | A | C |
| ATOM | 2184 | O | LEU | A | 282 | −30.002 | −74.340 | −20.151 | 1.00 | 21.89 | A | O |
| ATOM | 2185 | N | TYR | A | 283 | −27.809 | −74.605 | −19.842 | 1.00 | 22.88 | A | N |
| ATOM | 2186 | CA | TYR | A | 283 | −27.545 | −75.030 | −21.212 | 1.00 | 22.90 | A | C |
| ATOM | 2187 | CB | TYR | A | 283 | −26.845 | −73.929 | −22.020 | 1.00 | 21.85 | A | C |
| ATOM | 2188 | CG | TYR | A | 283 | −27.559 | −72.574 | −21.968 | 1.00 | 21.84 | A | C |
| ATOM | 2189 | CD1 | TYR | A | 283 | −28.816 | −72.417 | −22.547 | 1.00 | 18.80 | A | C |
| ATOM | 2190 | CE1 | TYR | A | 283 | −29.461 | −71.200 | −22.491 | 1.00 | 20.76 | A | C |
| ATOM | 2191 | CZ | TYR | A | 283 | −28.874 | −70.092 | −21.858 | 1.00 | 20.12 | A | C |
| ATOM | 2192 | OH | TYR | A | 283 | −29.586 | −68.908 | −21.854 | 1.00 | 20.69 | A | O |
| ATOM | 2193 | CE2 | TYR | A | 283 | −27.652 | −70.195 | −21.253 | 1.00 | 15.68 | A | C |
| ATOM | 2194 | CD2 | TYR | A | 283 | −26.983 | −71.454 | −21.307 | 1.00 | 17.76 | A | C |
| ATOM | 2195 | C | TYR | A | 283 | −26.777 | −76.314 | −21.367 | 1.00 | 23.64 | A | C |
| ATOM | 2196 | O | TYR | A | 283 | −26.744 | −76.821 | −22.465 | 1.00 | 23.90 | A | O |
| ATOM | 2197 | N | GLY | A | 284 | −26.149 | −76.846 | −20.308 | 1.00 | 24.18 | A | N |
| ATOM | 2198 | CA | GLY | A | 284 | −25.490 | −78.146 | −20.441 | 1.00 | 23.74 | A | C |
| ATOM | 2199 | C | GLY | A | 284 | −23.995 | −78.012 | −20.547 | 1.00 | 25.56 | A | C |
| ATOM | 2200 | O | GLY | A | 284 | −23.429 | −76.878 | −20.400 | 1.00 | 26.07 | A | O |
| ATOM | 2201 | N | SER | A | 285 | −23.367 | −79.169 | −20.765 | 1.00 | 25.64 | A | N |
| ATOM | 2202 | CA | SER | A | 285 | −21.924 | −79.403 | −20.789 | 1.00 | 26.97 | A | C |
| ATOM | 2203 | CB | SER | A | 285 | −21.644 | −80.813 | −21.289 | 1.00 | 27.65 | A | C |
| ATOM | 2204 | OG | SER | A | 285 | −21.679 | −81.599 | −20.147 | 1.00 | 30.68 | A | O |
| ATOM | 2205 | C | SER | A | 285 | −21.060 | −78.586 | −21.678 | 1.00 | 26.93 | A | C |
| ATOM | 2206 | O | SER | A | 285 | −19.949 | −78.197 | −21.282 | 1.00 | 27.19 | A | O |
| ATOM | 2207 | N | GLN | A | 286 | −21.517 | −78.384 | −22.892 | 1.00 | 26.76 | A | N |
| ATOM | 2208 | CA | GLN | A | 286 | −20.648 | −77.835 | −23.874 | 1.00 | 28.08 | A | C |
| ATOM | 2209 | CB | GLN | A | 286 | −21.265 | −78.047 | −25.242 | 1.00 | 30.00 | A | C |
| ATOM | 2210 | CG | GLN | A | 286 | −20.317 | −78.539 | −26.290 | 1.00 | 39.99 | A | C |
| ATOM | 2211 | CD | GLN | A | 286 | −21.093 | −79.305 | −27.400 | 1.00 | 52.85 | A | C |
| ATOM | 2212 | OE1 | GLN | A | 286 | −22.302 | −79.063 | −27.620 | 1.00 | 56.21 | A | O |
| ATOM | 2213 | NE2 | GLN | A | 286 | −20.401 | −80.226 | −28.097 | 1.00 | 54.74 | A | N |
| ATOM | 2214 | C | GLN | A | 286 | −20.490 | −76.349 | −23.594 | 1.00 | 26.64 | A | C |
| ATOM | 2215 | O | GLN | A | 286 | −19.363 | −75.804 | −23.701 | 1.00 | 26.48 | A | O |
| ATOM | 2216 | N | ASP | A | 287 | −21.589 | −75.686 | −23.203 | 1.00 | 24.40 | A | N |
| ATOM | 2217 | CA | ASP | A | 287 | −21.488 | −74.240 | −22.904 | 1.00 | 22.97 | A | C |
| ATOM | 2218 | CB | ASP | A | 287 | −22.865 | −73.495 | −22.906 | 1.00 | 21.60 | A | C |
| ATOM | 2219 | CG | ASP | A | 287 | −23.535 | −73.501 | −24.311 | 1.00 | 24.04 | A | C |
| ATOM | 2220 | OD1 | ASP | A | 287 | −24.782 | −73.687 | −24.383 | 1.00 | 27.03 | A | O |
| ATOM | 2221 | OD2 | ASP | A | 287 | −22.824 | −73.346 | −25.351 | 1.00 | 23.58 | A | O |
| ATOM | 2222 | C | ASP | A | 287 | −20.686 | −74.062 | −21.631 | 1.00 | 21.52 | A | C |
| ATOM | 2223 | O | ASP | A | 287 | −19.872 | −73.146 | −21.556 | 1.00 | 20.96 | A | O |
| ATOM | 2224 | N | ALA | A | 288 | −20.869 | −74.979 | −20.663 | 1.00 | 21.28 | A | N |
| ATOM | 2225 | CA | ALA | A | 288 | −19.968 | −75.095 | −19.491 | 1.00 | 20.85 | A | C |
| ATOM | 2226 | CB | ALA | A | 288 | −20.373 | −76.242 | −18.555 | 1.00 | 20.98 | A | C |
| ATOM | 2227 | C | ALA | A | 288 | −18.502 | −75.224 | −19.849 | 1.00 | 20.23 | A | C |
| ATOM | 2228 | O | ALA | A | 288 | −17.687 | −74.457 | −19.337 | 1.00 | 23.40 | A | O |
| ATOM | 2229 | N | ALA | A | 289 | −18.134 | −76.136 | −20.728 | 1.00 | 18.74 | A | N |
| ATOM | 2230 | CA | ALA | A | 289 | −16.734 | −76.188 | −21.224 | 1.00 | 18.20 | A | C |
| ATOM | 2231 | CB | ALA | A | 289 | −16.517 | −77.355 | −22.253 | 1.00 | 15.30 | A | C |
| ATOM | 2232 | C | ALA | A | 289 | −16.224 | −74.821 | −21.797 | 1.00 | 18.09 | A | C |
| ATOM | 2233 | O | ALA | A | 289 | −15.079 | −74.402 | −21.525 | 1.00 | 19.17 | A | O |
| ATOM | 2234 | N | SER | A | 290 | −17.035 | −74.145 | −22.592 | 1.00 | 17.27 | A | N |
| ATOM | 2235 | CA | SER | A | 290 | −16.602 | −72.866 | −23.165 | 1.00 | 18.01 | A | C |
| ATOM | 2236 | CB | SER | A | 290 | −17.574 | −72.332 | −24.200 | 1.00 | 17.59 | A | C |
| ATOM | 2237 | OG | SER | A | 290 | −17.978 | −73.398 | −25.030 | 1.00 | 17.68 | A | O |
| ATOM | 2238 | C | SER | A | 290 | −16.430 | −71.814 | −22.096 | 1.00 | 17.84 | A | C |
| ATOM | 2239 | O | SER | A | 290 | −15.458 | −71.040 | −22.161 | 1.00 | 18.11 | A | O |
| ATOM | 2240 | N | VAL | A | 291 | −17.347 | −71.766 | −21.119 | 1.00 | 17.53 | A | N |
| ATOM | 2241 | CA | VAL | A | 291 | −17.137 | −70.846 | −19.979 | 1.00 | 16.01 | A | C |
| ATOM | 2242 | CB | VAL | A | 291 | −18.259 | −70.906 | −18.946 | 1.00 | 16.40 | A | C |
| ATOM | 2243 | CG1 | VAL | A | 291 | −17.867 | −70.159 | −17.668 | 1.00 | 12.35 | A | C |
| ATOM | 2244 | CG2 | VAL | A | 291 | −19.547 | −70.364 | −19.535 | 1.00 | 14.61 | A | C |
| ATOM | 2245 | C | VAL | A | 291 | −15.790 | −71.192 | −19.360 | 1.00 | 16.98 | A | C |
| ATOM | 2246 | O | VAL | A | 291 | −14.935 | −70.295 | −19.156 | 1.00 | 17.93 | A | O |
| ATOM | 2247 | N | GLU | A | 292 | −15.554 | −72.485 | −19.084 | 1.00 | 17.26 | A | N |
| ATOM | 2248 | CA | GLU | A | 292 | −14.285 | −72.893 | −18.404 | 1.00 | 17.43 | A | C |
| ATOM | 2249 | CB | GLU | A | 292 | −14.296 | −74.410 | −18.184 | 1.00 | 16.88 | A | C |
| ATOM | 2250 | CG | GLU | A | 292 | −13.056 | −74.909 | −17.514 | 1.00 | 18.64 | A | C |
| ATOM | 2251 | CD | GLU | A | 292 | −13.160 | −76.349 | −17.130 | 1.00 | 21.95 | A | C |
| ATOM | 2252 | OE1 | GLU | A | 292 | −13.129 | −77.179 | −18.042 | 1.00 | 22.49 | A | O |
| ATOM | 2253 | OE2 | GLU | A | 292 | −13.281 | −76.658 | −15.926 | 1.00 | 21.17 | A | O |
| ATOM | 2254 | C | GLU | A | 292 | −13.009 | −72.459 | −19.221 | 1.00 | 17.74 | A | C |
| ATOM | 2255 | O | GLU | A | 292 | −12.023 | −71.872 | −18.666 | 1.00 | 19.53 | A | O |
| ATOM | 2256 | N | ALA | A | 293 | −13.031 | −72.732 | −20.521 | 1.00 | 15.97 | A | N |
| ATOM | 2257 | CA | ALA | A | 293 | −11.959 | −72.339 | −21.435 | 1.00 | 16.10 | A | C |
| ATOM | 2258 | CB | ALA | A | 293 | −12.266 | −72.836 | −22.889 | 1.00 | 13.76 | A | C |
| ATOM | 2259 | C | ALA | A | 293 | −11.743 | −70.814 | −21.410 | 1.00 | 17.14 | A | C |
| ATOM | 2260 | O | ALA | A | 293 | −10.569 | −70.319 | −21.455 | 1.00 | 17.89 | A | O |
| ATOM | 2261 | N | ALA | A | 294 | −12.849 | −70.063 | −21.330 | 1.00 | 16.60 | A | N |
| ATOM | 2262 | CA | ALA | A | 294 | −12.746 | −68.599 | −21.268 | 1.00 | 17.10 | A | C |
| ATOM | 2263 | CB | ALA | A | 294 | −14.098 | −67.948 | −21.258 | 1.00 | 16.35 | A | C |
| ATOM | 2264 | C | ALA | A | 294 | −11.953 | −68.177 | −20.039 | 1.00 | 17.40 | A | C |
| ATOM | 2265 | O | ALA | A | 294 | −11.212 | −67.221 | −20.121 | 1.00 | 17.93 | A | O |
| ATOM | 2266 | N | TRP | A | 295 | −12.058 | −68.906 | −18.916 | 1.00 | 17.09 | A | N |
| ATOM | 2267 | CA | TRP | A | 295 | −11.301 | −68.546 | −17.702 | 1.00 | 16.03 | A | C |
| ATOM | 2268 | CB | TRP | A | 295 | −12.029 | −69.018 | −16.423 | 1.00 | 16.23 | A | C |
| ATOM | 2269 | CG | TRP | A | 295 | −13.219 | −68.152 | −16.073 | 1.00 | 13.59 | A | C |
| ATOM | 2270 | CD1 | TRP | A | 295 | −14.511 | −68.249 | −16.584 | 1.00 | 12.53 | A | C |
| ATOM | 2271 | NE1 | TRP | A | 295 | −15.301 | −67.226 | −16.047 | 1.00 | 16.35 | A | N |
| ATOM | 2272 | CE2 | TRP | A | 295 | −14.543 | −66.484 | −15.161 | 1.00 | 14.53 | A | C |
| ATOM | 2273 | CD2 | TRP | A | 295 | −13.226 | −67.016 | −15.169 | 1.00 | 12.86 | A | C |
| ATOM | 2274 | CE3 | TRP | A | 295 | −12.249 | −66.411 | −14.357 | 1.00 | 12.83 | A | C |
| ATOM | 2275 | CZ3 | TRP | A | 295 | −12.591 | −65.298 | −13.575 | 1.00 | 11.80 | A | C |
| ATOM | 2276 | CH2 | TRP | A | 295 | −13.910 | −64.782 | −13.569 | 1.00 | 12.57 | A | C |
| ATOM | 2277 | CZ2 | TRP | A | 295 | −14.894 | −65.343 | −14.376 | 1.00 | 15.73 | A | C |
| ATOM | 2278 | C | TRP | A | 295 | −9.895 | −69.097 | −17.803 | 1.00 | 17.18 | A | C |
| ATOM | 2279 | O | TRP | A | 295 | −8.914 | −68.446 | −17.386 | 1.00 | 18.22 | A | O |
| ATOM | 2280 | N | ASN | A | 296 | −9.749 | −70.269 | −18.417 | 1.00 | 16.56 | A | N |
| ATOM | 2281 | CA | ASN | A | 296 | −8.391 | −70.769 | −18.692 | 1.00 | 15.21 | A | C |
| ATOM | 2282 | CB | ASN | A | 296 | −8.457 | −72.131 | −19.424 | 1.00 | 14.65 | A | C |
| ATOM | 2283 | CG | ASN | A | 296 | −9.033 | −73.285 | −18.512 | 1.00 | 16.77 | A | C |
| ATOM | 2284 | OD1 | ASN | A | 296 | −9.175 | −73.141 | −17.276 | 1.00 | 20.24 | A | O |
| ATOM | 2285 | ND2 | ASN | A | 296 | −9.387 | −74.380 | −19.120 | 1.00 | 14.16 | A | N |
| ATOM | 2286 | C | ASN | A | 296 | −7.521 | −69.729 | −19.432 | 1.00 | 16.44 | A | C |
| ATOM | 2287 | O | ASN | A | 296 | −6.303 | −69.529 | −19.113 | 1.00 | 16.54 | A | O |
| ATOM | 2288 | N | ALA | A | 297 | −8.126 | −69.035 | −20.406 | 1.00 | 16.57 | A | N |
| ATOM | 2289 | CA | ALA | A | 297 | −7.355 | −68.195 | −21.309 | 1.00 | 16.69 | A | C |
| ATOM | 2290 | CB | ALA | A | 297 | −8.181 | −67.747 | −22.564 | 1.00 | 15.79 | A | C |
| ATOM | 2291 | C | ALA | A | 297 | −6.858 | −66.990 | −20.556 | 1.00 | 17.86 | A | C |
| ATOM | 2292 | O | ALA | A | 297 | −5.951 | −66.356 | −21.041 | 1.00 | 19.04 | A | O |
| ATOM | 2293 | N | VAL | A | 298 | −7.426 | −66.663 | −19.388 | 1.00 | 18.30 | A | N |
| ATOM | 2294 | CA | VAL | A | 298 | −6.891 | −65.550 | −18.595 | 1.00 | 19.09 | A | C |
| ATOM | 2295 | CB | VAL | A | 298 | −7.981 | −64.544 | −18.074 | 1.00 | 19.27 | A | C |
| ATOM | 2296 | CG1 | VAL | A | 298 | −8.904 | −64.123 | −19.182 | 1.00 | 18.35 | A | C |
| ATOM | 2297 | CG2 | VAL | A | 298 | −8.752 | −65.155 | −16.892 | 1.00 | 17.14 | A | C |
| ATOM | 2298 | C | VAL | A | 298 | −6.069 | −66.035 | −17.372 | 1.00 | 20.71 | A | C |
| ATOM | 2299 | O | VAL | A | 298 | −5.776 | −65.201 | −16.456 | 1.00 | 21.47 | A | O |
| ATOM | 2300 | N | GLY | A | 299 | −5.710 | −67.337 | −17.362 | 1.00 | 18.91 | A | N |
| ATOM | 2301 | CA | GLY | A | 299 | −4.856 | −67.900 | −16.345 | 1.00 | 18.24 | A | C |
| ATOM | 2302 | C | GLY | A | 299 | −5.536 | −68.500 | −15.113 | 1.00 | 19.14 | A | C |
| ATOM | 2303 | O | GLY | A | 299 | −4.903 | −68.602 | −14.071 | 1.00 | 19.70 | A | O |
| ATOM | 2304 | N | LEU | A | 300 | −6.801 | −68.912 | −15.215 | 1.00 | 19.37 | A | N |
| ATOM | 2305 | CA | LEU | A | 300 | −7.579 | −69.370 | −14.049 | 1.00 | 19.59 | A | C |
| ATOM | 2306 | CB | LEU | A | 300 | −8.594 | −68.309 | −13.572 | 1.00 | 18.80 | A | C |
| ATOM | 2307 | CG | LEU | A | 300 | −8.010 | −67.213 | −12.664 | 1.00 | 20.17 | A | C |
| ATOM | 2308 | CD1 | LEU | A | 300 | −8.820 | −65.889 | −12.682 | 1.00 | 14.68 | A | C |
| ATOM | 2309 | CD2 | LEU | A | 300 | −7.730 | −67.747 | −11.152 | 1.00 | 15.80 | A | C |
| ATOM | 2310 | C | LEU | A | 300 | −8.321 | −70.657 | −14.342 | 1.00 | 20.21 | A | C |
| ATOM | 2311 | O | LEU | A | 300 | −8.607 | −70.984 | −15.546 | 1.00 | 19.40 | A | O |
| ATOM | 2312 | OXT | LEU | A | 300 | −8.659 | −71.329 | −13.331 | 1.00 | 19.54 | A | O |
| ATOM | 2313 | ZN | ZN | A | 325 | −18.474 | −49.581 | −10.110 | 1.00 | 26.57 | A | ZN |
| ATOM | 2314 | CA | CA | A | 326 | −20.141 | −49.094 | −24.614 | 1.00 | 21.37 | A | CA |
| ATOM | 2315 | CA | CA | A | 327 | −21.620 | −47.425 | −27.379 | 1.00 | 13.56 | A | CA |
| ATOM | 2316 | O8 | BTB | A | 401 | −25.754 | −64.686 | −4.944 | 1.00 | 33.84 | | O |
| ATOM | 2317 | C8 | BTB | A | 401 | −27.177 | −64.941 | −4.724 | 1.00 | 49.20 | | C |
| ATOM | 2318 | C7 | BTB | A | 401 | −27.961 | −64.219 | −3.595 | 1.00 | 48.31 | | C |
| ATOM | 2319 | N | BTB | A | 401 | −28.068 | −65.122 | −2.424 | 1.00 | 50.93 | | N |
| ATOM | 2320 | C5 | BTB | A | 401 | −29.458 | −65.487 | −2.106 | 1.00 | 53.08 | | C |
| ATOM | 2321 | C6 | BTB | A | 401 | −30.027 | −64.461 | −1.056 | 1.00 | 54.37 | | C |
| ATOM | 2322 | O6 | BTB | A | 401 | −29.100 | −63.396 | −0.674 | 1.00 | 51.50 | | O |
| ATOM | 2323 | C2 | BTB | A | 401 | −26.911 | −65.600 | −1.545 | 1.00 | 50.97 | | C |
| ATOM | 2324 | C4 | BTB | A | 401 | −25.924 | −64.439 | −1.315 | 1.00 | 49.99 | | C |
| ATOM | 2325 | O4 | BTB | A | 401 | −25.290 | −64.470 | −0.033 | 1.00 | 50.81 | | O |
| ATOM | 2326 | C3 | BTB | A | 401 | −26.124 | −66.761 | −2.205 | 1.00 | 49.77 | | C |
| ATOM | 2327 | O3 | BTB | A | 401 | −25.471 | −67.594 | −1.239 | 1.00 | 49.48 | | O |
| ATOM | 2328 | C1 | BTB | A | 401 | −27.471 | −66.012 | −0.165 | 1.00 | 51.74 | | C |
| ATOM | 2329 | O1 | BTB | A | 401 | −28.588 | −66.951 | −0.241 | 1.00 | 51.00 | | O |
| ATOM | 2330 | O | HOH | C | 2 | −2.711 | −67.592 | −19.132 | 1.00 | 29.87 | | O |
| ATOM | 2331 | O | HOH | C | 3 | −16.211 | −62.592 | −15.497 | 1.00 | 13.75 | | O |
| ATOM | 2332 | O | HOH | C | 5 | −13.080 | −72.566 | −9.617 | 1.00 | 25.61 | | O |
| ATOM | 2333 | O | HOH | C | 6 | −12.806 | −53.340 | −28.915 | 1.00 | 18.86 | | O |
| ATOM | 2334 | O | HOH | C | 7 | −2.312 | −58.186 | −18.387 | 1.00 | 21.39 | | O |
| ATOM | 2335 | O | HOH | C | 8 | −17.631 | −66.593 | −17.691 | 1.00 | 14.22 | | O |
| ATOM | 2336 | O | HOH | C | 9 | −35.936 | −48.250 | −3.867 | 1.00 | 19.09 | | O |
| ATOM | 2337 | O | HOH | C | 10 | −20.260 | −73.864 | −26.521 | 1.00 | 21.53 | | O |
| ATOM | 2338 | O | HOH | C | 11 | −7.569 | −64.056 | −22.670 | 1.00 | 16.33 | | O |
| ATOM | 2339 | O | HOH | C | 12 | −41.422 | −26.102 | −22.458 | 1.00 | 36.88 | | O |
| ATOM | 2340 | O | HOH | C | 13 | −2.583 | −67.718 | −13.855 | 1.00 | 19.81 | | O |
| ATOM | 2341 | O | HOH | C | 14 | −42.378 | −48.644 | −20.422 | 1.00 | 24.29 | | O |
| ATOM | 2342 | O | HOH | C | 15 | −40.574 | −61.262 | −18.180 | 1.00 | 38.06 | | O |
| ATOM | 2343 | O | HOH | C | 16 | −30.259 | −66.532 | −15.709 | 1.00 | 21.74 | | O |
| ATOM | 2344 | O | HOH | C | 17 | −28.285 | −38.912 | −27.320 | 1.00 | 17.16 | | O |
| ATOM | 2345 | O | HOH | C | 18 | −32.187 | −62.257 | −7.228 | 1.00 | 16.83 | | O |
| ATOM | 2346 | O | HOH | C | 19 | −25.078 | −44.158 | −23.968 | 1.00 | 14.65 | | O |
| ATOM | 2347 | O | HOH | C | 20 | −23.895 | −46.936 | −23.545 | 1.00 | 16.14 | | O |
| ATOM | 2348 | O | HOH | C | 21 | −41.667 | −48.219 | −7.746 | 1.00 | 34.18 | | O |
| ATOM | 2349 | O | HOH | C | 22 | −40.068 | −48.275 | −3.726 | 1.00 | 19.95 | | O |
| ATOM | 2350 | O | HOH | C | 23 | −35.010 | −62.824 | −11.601 | 1.00 | 26.66 | | O |
| ATOM | 2351 | O | HOH | C | 24 | −13.021 | −76.519 | −21.028 | 1.00 | 30.07 | | O |
| ATOM | 2352 | O | HOH | C | 25 | −19.087 | −36.433 | −24.374 | 1.00 | 34.42 | | O |
| ATOM | 2353 | O | HOH | C | 26 | −24.793 | −80.040 | −12.678 | 1.00 | 29.84 | | O |
| ATOM | 2354 | O | HOH | C | 27 | −30.660 | −63.583 | −22.716 | 1.00 | 19.99 | | O |
| ATOM | 2355 | O | HOH | C | 28 | −24.403 | −76.833 | −24.011 | 1.00 | 25.02 | | O |
| ATOM | 2356 | O | HOH | C | 29 | −1.256 | −55.688 | −17.362 | 1.00 | 25.31 | | O |
| ATOM | 2357 | O | HOH | C | 30 | −31.120 | −63.745 | −5.118 | 1.00 | 25.53 | | O |
| ATOM | 2358 | O | HOH | C | 31 | −32.816 | −71.675 | −9.104 | 1.00 | 42.94 | | O |
| ATOM | 2359 | O | HOH | C | 32 | −25.511 | −47.418 | −25.772 | 1.00 | 30.04 | | O |
| ATOM | 2360 | O | HOH | C | 33 | 2.080 | −61.199 | −7.018 | 1.00 | 32.19 | | O |
| ATOM | 2361 | O | HOH | C | 34 | −10.798 | −60.304 | 0.807 | 1.00 | 34.30 | | O |
| ATOM | 2362 | O | HOH | C | 35 | −13.969 | −52.276 | −13.493 | 1.00 | 22.29 | | O |
| ATOM | 2363 | O | HOH | C | 36 | −26.801 | −37.860 | −24.720 | 1.00 | 24.12 | | O |
| ATOM | 2364 | O | HOH | C | 37 | −20.776 | −45.215 | −22.957 | 1.00 | 21.88 | | O |
| ATOM | 2365 | O | HOH | C | 38 | 1.055 | −57.909 | −10.764 | 1.00 | 16.66 | | O |
| ATOM | 2366 | O | HOH | C | 39 | −32.697 | −59.896 | −4.934 | 1.00 | 23.95 | | O |
| ATOM | 2367 | O | HOH | C | 40 | −33.179 | −53.385 | −12.401 | 1.00 | 27.60 | | O |
| ATOM | 2368 | O | HOH | C | 41 | −7.976 | −52.645 | −29.054 | 1.00 | 32.74 | | O |
| ATOM | 2369 | O | HOH | C | 42 | −24.307 | −79.767 | −24.391 | 1.00 | 35.33 | | O |
| ATOM | 2370 | O | HOH | C | 43 | −38.839 | −42.447 | −20.245 | 1.00 | 21.37 | | O |
| ATOM | 2371 | O | HOH | C | 44 | −52.198 | −32.387 | −13.024 | 1.00 | 34.97 | | O |
| ATOM | 2372 | O | HOH | C | 45 | −33.718 | −45.777 | −6.287 | 1.00 | 21.53 | | O |
| ATOM | 2373 | O | HOH | C | 46 | −5.504 | −62.986 | −21.066 | 1.00 | 33.17 | | O |
| ATOM | 2374 | O | HOH | C | 47 | −18.734 | −64.795 | 2.288 | 1.00 | 34.55 | | O |
| ATOM | 2375 | O | HOH | C | 48 | −24.367 | −38.933 | −24.187 | 1.00 | 26.39 | | O |
| ATOM | 2376 | O | HOH | C | 49 | −43.788 | −47.175 | −13.701 | 1.00 | 27.38 | | O |
| ATOM | 2377 | O | HOH | C | 50 | −26.131 | −66.593 | −26.975 | 1.00 | 31.01 | | O |
| ATOM | 2378 | O | HOH | C | 51 | −22.554 | −53.372 | −4.818 | 1.00 | 25.37 | | O |
| ATOM | 2379 | O | HOH | C | 52 | −38.611 | −43.353 | −22.739 | 1.00 | 28.58 | | O |
| ATOM | 2380 | O | HOH | C | 53 | 1.781 | −56.450 | −8.391 | 1.00 | 20.44 | | O |
| ATOM | 2381 | O | HOH | C | 54 | −9.763 | −51.219 | −19.497 | 1.00 | 21.78 | | O |
| ATOM | 2382 | O | HOH | C | 55 | −35.947 | −54.526 | −13.693 | 1.00 | 29.02 | | O |
| ATOM | 2383 | O | HOH | C | 56 | −34.353 | −65.294 | −14.056 | 1.00 | 32.29 | | O |
| ATOM | 2384 | O | HOH | C | 57 | 0.430 | −61.703 | −8.963 | 1.00 | 31.79 | | O |
| ATOM | 2385 | O | HOH | C | 58 | −46.066 | −43.418 | −20.148 | 1.00 | 39.75 | | O |
| ATOM | 2386 | O | HOH | C | 59 | −9.719 | −55.069 | −28.478 | 1.00 | 22.91 | | O |
| ATOM | 2387 | O | HOH | C | 60 | −11.594 | −47.814 | −26.606 | 1.00 | 28.37 | | O |
| ATOM | 2388 | O | HOH | C | 61 | −27.425 | −59.413 | −0.115 | 1.00 | 30.47 | | O |
| ATOM | 2389 | O | HOH | C | 62 | −25.237 | −34.589 | −13.754 | 1.00 | 25.20 | | O |
| ATOM | 2390 | O | HOH | C | 63 | −24.889 | −76.446 | −26.846 | 1.00 | 39.30 | | O |
| ATOM | 2391 | O | HOH | C | 64 | −28.189 | −67.960 | −28.520 | 1.00 | 9.69 | | O |
| ATOM | 2392 | O | HOH | C | 65 | 2.279 | −57.488 | −13.341 | 1.00 | 22.09 | | O |
| ATOM | 2393 | O | HOH | C | 66 | −9.170 | −66.952 | −6.893 | 1.00 | 17.86 | | O |
| ATOM | 2394 | O | HOH | C | 67 | −18.314 | −47.411 | −9.661 | 1.00 | 20.32 | | O |
| ATOM | 2395 | O | HOH | C | 68 | −31.399 | −68.090 | −23.976 | 1.00 | 28.09 | | O |
| ATOM | 2396 | O | HOH | C | 69 | −12.106 | −51.680 | −27.036 | 1.00 | 14.12 | | O |
| ATOM | 2397 | O | HOH | C | 70 | −17.481 | −60.702 | −14.176 | 1.00 | 25.54 | | O |
| ATOM | 2398 | O | HOH | C | 71 | −1.207 | −68.518 | −16.338 | 1.00 | 21.45 | | O |
| ATOM | 2399 | O | HOH | C | 72 | −40.653 | −60.453 | −10.248 | 1.00 | 45.50 | | O |
| ATOM | 2400 | O | HOH | C | 73 | −17.827 | −46.310 | −13.499 | 1.00 | 31.29 | | O |
| ATOM | 2401 | O | HOH | C | 74 | −23.273 | −80.385 | −17.437 | 1.00 | 38.60 | | O |
| ATOM | 2402 | O | HOH | C | 75 | −11.710 | −54.235 | −18.487 | 1.00 | 12.63 | | O |
| ATOM | 2403 | O | HOH | C | 76 | −37.268 | −63.184 | −10.316 | 1.00 | 34.47 | | O |
| ATOM | 2404 | O | HOH | C | 77 | −40.855 | −53.336 | −19.367 | 1.00 | 40.20 | | O |
| ATOM | 2405 | O | HOH | C | 78 | −15.231 | −52.071 | −29.251 | 1.00 | 14.34 | | O |
| ATOM | 2406 | O | HOH | C | 79 | −18.374 | −75.243 | −10.951 | 1.00 | 32.51 | | O |
| ATOM | 2407 | O | HOH | C | 80 | −8.556 | −71.510 | −23.158 | 1.00 | 27.00 | | O |
| ATOM | 2408 | O | HOH | C | 81 | −39.663 | −31.157 | −5.844 | 1.00 | 41.74 | | O |
| ATOM | 2409 | O | HOH | C | 82 | −27.219 | −53.428 | −26.557 | 1.00 | 32.84 | | O |
| ATOM | 2410 | O | HOH | C | 83 | −14.922 | −49.381 | −29.521 | 1.00 | 26.74 | | O |
| ATOM | 2411 | O | HOH | C | 84 | −9.883 | −70.265 | −25.549 | 1.00 | 31.60 | | O |
| ATOM | 2412 | O | HOH | C | 85 | −6.047 | −71.565 | −22.212 | 1.00 | 27.12 | | O |
| ATOM | 2413 | O | HOH | C | 86 | −4.894 | −73.457 | −20.619 | 1.00 | 24.66 | | O |
| ATOM | 2414 | O | HOH | C | 87 | −9.284 | −61.962 | −2.485 | 1.00 | 18.79 | | O |
| ATOM | 2415 | O | HOH | C | 88 | −22.712 | −48.116 | −28.995 | 1.00 | 23.45 | | O |
| ATOM | 2416 | O | HOH | C | 89 | −20.620 | −51.414 | −24.485 | 1.00 | 19.66 | | O |
| ATOM | 2417 | O | HOH | C | 90 | −23.556 | −62.487 | −29.443 | 1.00 | 22.98 | | O |
| ATOM | 2418 | O | HOH | C | 91 | −20.711 | −46.214 | −28.623 | 1.00 | 18.98 | | O |
| ATOM | 2419 | O | HOH | C | 92 | −8.725 | −56.266 | −6.821 | 1.00 | 31.39 | | O |
| ATOM | 2420 | O | HOH | C | 93 | −22.021 | −55.134 | −9.353 | 1.00 | 26.24 | | O |
| ATOM | 2421 | O | HOH | C | 94 | −23.737 | −28.744 | −1.310 | 1.00 | 41.74 | | O |
| ATOM | 2422 | O | HOH | C | 95 | −6.811 | −49.921 | −8.253 | 1.00 | 35.42 | | O |
| ATOM | 2423 | O | HOH | C | 96 | −25.425 | −53.168 | −35.560 | 1.00 | 24.85 | | O |
| ATOM | 2424 | O | HOH | C | 97 | −31.444 | −62.742 | −2.659 | 1.00 | 35.46 | | O |
| ATOM | 2425 | O | HOH | C | 98 | −6.736 | −58.471 | −24.600 | 1.00 | 38.37 | | O |
| ATOM | 2426 | O | HOH | C | 99 | −28.262 | −30.941 | −18.263 | 1.00 | 25.36 | | O |
| ATOM | 2427 | O | HOH | C | 100 | −25.294 | −36.174 | −16.058 | 1.00 | 28.89 | | O |
| ATOM | 2428 | O | HOH | C | 101 | −34.158 | −41.383 | −12.513 | 1.00 | 36.88 | | O |
| ATOM | 2429 | O | HOH | C | 102 | −20.678 | −59.982 | −28.773 | 1.00 | 32.58 | | O |
| ATOM | 2430 | O | HOH | C | 103 | −23.104 | −45.976 | −26.531 | 1.00 | 19.37 | | O |
| ATOM | 2431 | O | HOH | C | 104 | −14.860 | −50.263 | −12.303 | 1.00 | 37.60 | | O |
| ATOM | 2432 | O | HOH | C | 105 | −21.078 | −29.119 | −12.350 | 1.00 | 40.95 | | O |
| ATOM | 2433 | O | HOH | C | 106 | −38.049 | −43.845 | −18.170 | 1.00 | 28.92 | | O |
| ATOM | 2434 | O | HOH | C | 107 | −20.238 | −77.224 | −12.325 | 1.00 | 29.15 | | O |
| ATOM | 2435 | O | HOH | C | 108 | −1.438 | −48.204 | 1.601 | 1.00 | 28.53 | | O |
| ATOM | 2436 | O | HOH | C | 109 | −5.758 | −57.592 | −3.844 | 1.00 | 37.72 | | O |
| ATOM | 2438 | O | HOH | C | 111 | −36.779 | −29.048 | −13.433 | 1.00 | 33.74 | | O |
| ATOM | 2439 | O | HOH | C | 112 | −31.245 | −68.295 | −13.382 | 1.00 | 27.31 | | O |
| ATOM | 2440 | O | HOH | C | 113 | −4.425 | −45.328 | 2.720 | 1.00 | 36.16 | | O |
| ATOM | 2441 | O | HOH | C | 114 | −13.705 | −60.485 | 1.229 | 1.00 | 34.26 | | O |
| ATOM | 2442 | O | HOH | C | 115 | −21.165 | −32.036 | −10.846 | 1.00 | 44.52 | | O |
| ATOM | 2444 | O | HOH | C | 117 | −38.547 | −52.247 | −2.195 | 1.00 | 23.00 | | O |
| END | | | | | | | | | | | | |

### Example 6

### Comparison of NprE Variant Structure with Thermolysin

A structure based sequence alignment of NprE variant with other metalloproteases is shown in FIG. 12. Mature protein sequences of metalloproteases Thermolysin (Bacillus thermoproteolyticus, protein 1), PbaPro1 (Paenibacillus barcinonensis, protein 1), PhuPro1 (Paenibacillus hunanensis, protein 1), PhuPro2 (Paenibacillus hunanensis, protein 2), PehPro1 ((Paenibacillus ehimensis, protein 1, Peh1.A crystal structure), PpoPro1 (Paenibacillus polymyxa, protein 1), PspPro3 (Paenibacillus sp., protein 3), and PspPro2 (Paenibacillus sp., protein 2), and BbrPro1 (Brevibacillus brevis, protein 1) were aligned with sequences of known Paenibacillus metalloproteases from P. polymyxa (PpoPro2, YP_003872179.1), P. peoriae (PpePro1, ZP_10241029.1), P. terrae (PtePro1, F5LRG4) and known neutral protease homologs 1NPC.A (Bacillus cereus metalloprotease) and 1 KEI.A (Thermolysin, Bacillus thermoproteolyticus). This alignment shows that the NprE variant shares a common deletion with PehPro1 and PpoPro2 relative to Thermolysin (pdb entry 1KEI).

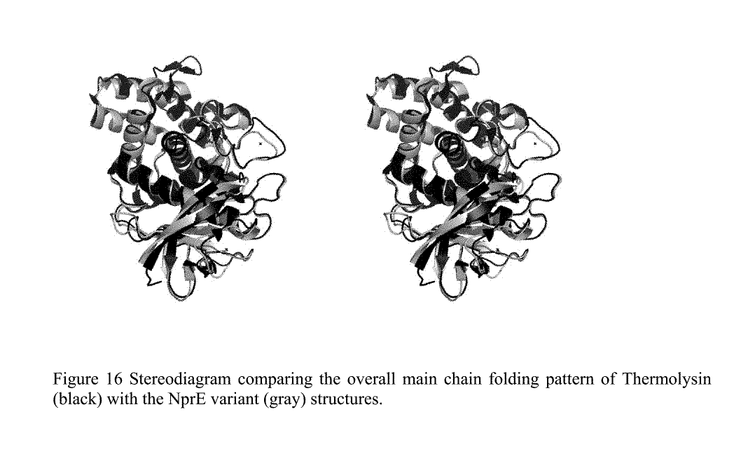

In the sequence of native NprE and NprE variant (shown), this five residue deletion occurs after residue Asp178 (NprE numbering). Seven residues in Thermolysin are replaced by Thr179-Glu180 of NprE. In addition, NprE variant manifests three other deletions, a 3-residues deletion after Ser191, a 10-residues deletion after Thr243 and a 2-residues deletion after Gly284, and three insertions of two residues after Lys33, one residue after Tyr49 and four residues after Pro217 (NprE numbering) not common with PehPro1 and PpoPro2. Nevertheless, the overall topology between NprE variant and Thermolysin is highly conserved as shown in FIG. 16, and also conserved in PehPro1 and PpoPro2 (not shown).

### Example 7

### Differences in Calcium Binding Sites Between Various Metalloproteases

In the electron density from the crystal of the NprE variant, there is density for two calcium ions, corresponding to Ca1,2 (the double calcium in Thermolysin described above). There is no electron density for calcium ions at sites Ca3 or Ca4 that are seen in Thermolysin. Note that the Ca4 site is conserved in both PehPro1 and PpoPro2, and the Ca3 site is also seen in the PpoPro2 structure as noted previously.

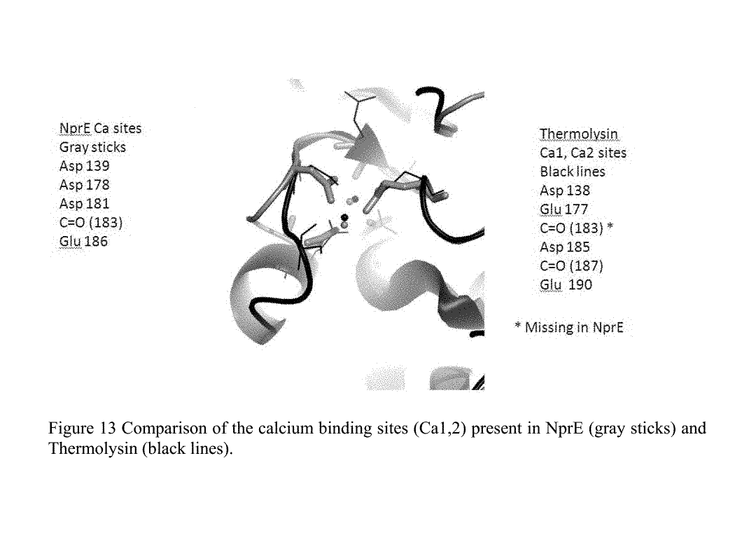

In the vicinity of the Thermolysin double calcium site (Ca1,2) the structure of the NprE variant retains a similar binding pattern as shown in FIG. 13. Despite having similar deletions as seen in PehPro1 and PpoPro2, the constellation of ligands is with one exception maintained by the presence of Asp181 which along with Glu186 (NprE numbering) forms ligands to both calcium ions. The single exception is that the carbonyl 0 of residue 183 in Thermolysin is absent owing to the deletion that is present in the NprE and common to PehPro1 and PpoPro2 structures. The carboxylates of Asp185 and Glu190 in Thermolysin form ligands to both calcium ions in Thermolysin and this pattern is retained in NprE with Asp181 and Glu186.

Referring back to FIG. 9, the difference in calcium binding seen between Thermolysin, NprE variant, PpoPro2 and PehPro1 can be attributed to the specific sequence differences as well as the presence of the five residue deletion. In Thermolysin, the Ca1-2 site is created by six ligands, while in NprE five of these ligands are retained, the last being removed due to the five residue deletion. Specifically, Asp181 in NprE and Asp185 in Thermolysin are replaced by Asn173 in PehPro1 and PpoPro2, Glu 186 in NprE and Glu190 in Thermolysin are replaced by Asn178 in PehPro1 and PpoPro2 (PehPro1 or PpoPro2 numbering, mature sequence). This deletion in PehPro1 and PpoPro2 removes three potential ligands. In addition, there is the removal of a main chain carbonyl (Asn183 in Thermolysin) and the presence of a new ligand (Asp129) in PehPro1 and PpoPro2 results in an altered calcium binding site in the PehPro1 and PpoPro2 enzyme structures, instead of the previously observed double site Ca1-2. In PehPro1 and PpoPro2, Asp129 replaces Gly and Ser found in Thermolysin and the NprE variant respectively. This site in PehPro1 and PpoPro2 has effectively been re-sculpted to form a single Ca binding site arising from the reduction of six to four calcium ligands.

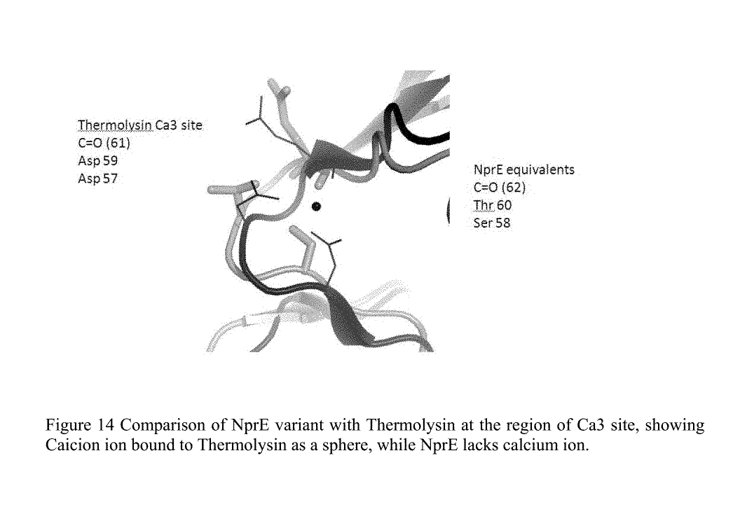

It is interesting that with the replacement of Asp57 and Asp59 (thermolysin numbering) in PehPro1 the D×D motif present in the majority of the M4 metalloprotease is now replaced by SSS/N at the “Ca3” site and PehPro1 does not have calcium binding at this site (FIG. 7 and FIG. 11). While the main chain conformation is conserved at the D×D site, aspartic acids at these positions are replaced with serine residues in PehPro1. In the structure of the NprE variant (FIG. 14), although the main chain conformation is again conserved, the aspartic acid residues observed in Thermolysin are now replaced by Ser and Thr residues, and calcium binding is absent.

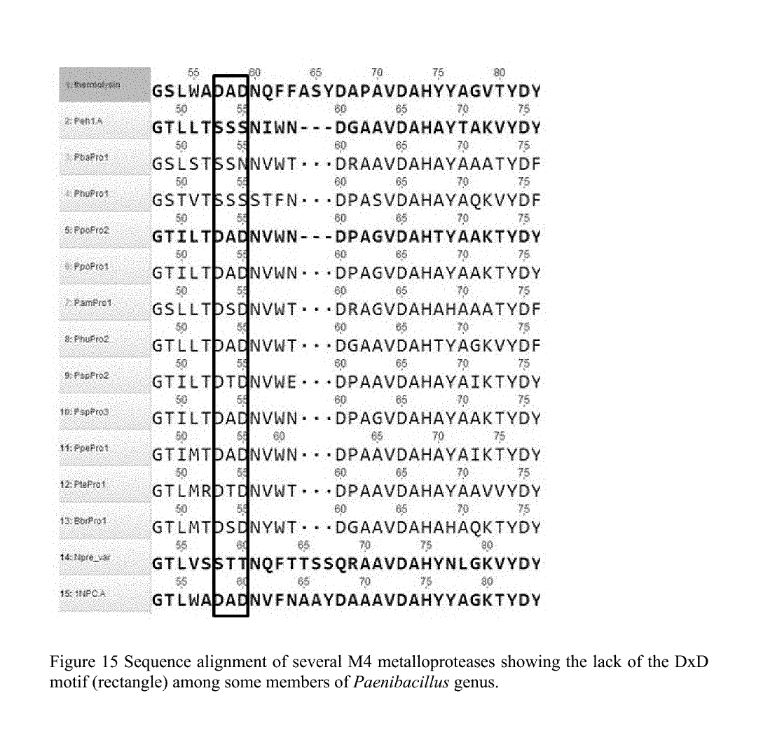

From multiple sequence alignment shown in FIG. 12, it is apparent that based on the presence of the D×D motif (the Ca3 site in Thermolysin and the third calcium site in PpoPro2), there seem to be different subsets of metalloproteases. Metalloproteases that have the D×D motif are exemplified by Thermolysin, the Bacillus cereus neutral protease and PpoPro2, while those that do not have the D×D motif have members such as PehPro1 and NprE. We see that several of proteases that contain the double delete lack the D×D motif (FIG. 15—blowup of FIG. 12).

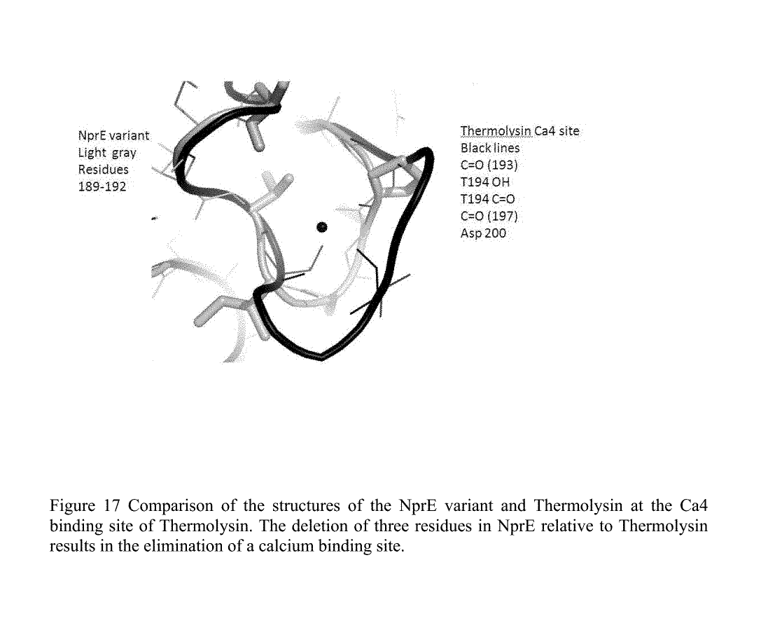

Thermolysin, PpoPro2 and PehPro1 all share one common Ca binding site, the Ca4 site in Thermolysin (See FIGS. 6 and 10). In the NprE variant, this site is absent owing to a deletion of three residues following Ser191 in the NprE variant relative to Thermolysin, PehPro1 and PpoPro2. A comparison of the structure of Thermolysin and the NprE variant are shown in FIG. 17.

### Example 8

### Strategy to Eliminate Calcium Dependence in Metalloproteases

The fundamental teaching derived from attempts to remove calcium from proteases such as subtilisin and Thermolysin is that removal of calcium is detrimental to stability folding and function of these enzymes. However, to use these proteases in detergents, one desires that the proteases function in a low calcium environment. Presence of calcium in high concentration is considered undesirable as it affects the hardness of water. Detergent manufacturers add chelating agent to remove dissolved calcium. Not wishing to be bound by theory, it is proposed that calcium dependence may be a primary contributor to the instability of metalloproteases, such as Thermolysin, in detergent solutions. Thus, a metalloprotease that requires less calcium for folding and stability would likely be more stable in detergent formulations.

One approach would be to begin with an enzyme that naturally had fewer calcium ions bound such as those manifesting the pattern of double delete metalloproteases or one lacking D×D motif.

In one embodiment, the strategy for removing the Thermolysin Ca4 calcium would follow the homology seen for NprE, namely, to replace residues at sites equivalent to residues 193-200 (YTPGISGD (SEQ ID NO:17)) in Thermolysin with the sequence TISQP (SEQ ID NO:18) present in NprE resulting in a three residue deletion.

The strategy for removing the site corresponding to Ca3 in Thermolysin would be to replace residues at sites equivalent to residues 55-66 (WADADNQFFASY (SEQ ID NO:19)) in Thermolysin with either a sequence replacing the D×D motif (i.e. WASSSNQFFASY (SEQ ID NO:20)) or replacing the D×D motif in Thermolysin along with the PehPro1 type deletion (i.e. LTSSSNIWN (SEQ ID NO:21)).

The strategy for removing the double calcium site in Thermolysin (Ca1,2) could be staged, first to replace the double site with a single site as found in PehPro1 and PpoPro2. Replacing the residues in Thermolysin at sites equivalent to residues 177-185 (EFYANKNPD (SEQ ID NO:22)) with DGKN (SEQ ID NO:23) in PehPro1 (resulting in a five residue deletion) along with the substitutions of the residue equivalent to Thermolysin position 136 (G) with Asp and Thermolysin position 190 (E) to Asp would result in the loss of one calcium.

To replace the remaining calcium ion, one additional step would be to replace the residue equivalent to Thermolysin position 136 with Lys or Arg so as to stabilize the negative charge present at the position equivalent to position 190 now an Asp. An additional step may be required to replace the residues equivalent to Thermolysin residues 177 (now Asp from above) to Asn or Ser and Thermolysin 138 (Asp) to Ser.

PROTEIN SEQUENCES FOR SEQUENCE LISTING:

PehPro1 (Paenibacillus ehimensis, protein 1)
ATGTGKGVLGDTKSETTTQSGSTYQLKDTTRGQGIVTYSAGNRSSLPGTL
LTSSSNIWNDGAAVDAHAYTAKVYDYYKNKFGRNSIDGNGFQLKSTVHYS
SRYNNAFWNGVQMVYGDGDGVTFIPFSADPDVIGHELTHGVTEHTAGLEY
YGESGALNESISDIIGNAIDGKNWLIGDLIYTPNTPGDALRSMENPKLYN
QPDRYQDRYTGPSDNGGVHINSGINNKAFYLIAQGGTHYGVTVNGIGRDA
AVQIFYDALINYLTPTSNESAMRAAAIQAATDLYGANSSQVNAVKKAYTA
VGVN (SEQ ID NO: 1)
PbaPro1 (Paenibacillus barcinonensis, protein 1)
ATGTGTGVHGDTKTLTTTQSGSTYQLKDTTRGKGIQTYTANNRSSLPGSL
STSSNNVWTDRAAVDAHAYAAATYDFYKNKENRNGIDGNGLLIRSTVHYG
SNYKNAFWNGAQIVYGDGDGIEFGPFSGDLDVVGHELTHGVIEYTANLEY
RNEPGALNEAFADIMGNTIESKNWLLGDGIYTPNIPGDALRSLSDPTLYN
QPDKYSDRYTGSQDNGGVHINSGIINKAYYLAAQGGTHNGVTVSGIGRDK
AVRIFYSTLVNYLTPTSKFAAAKTATIQAAKDLYGANSAEATAITKAYQA
VGL (SEQ ID NO: 2)
PhuPro1 (Paenibacillus hunanensis, protein 1)
ATGTGKGVLGDTKSFTVGTSGSSYVMTDSTRGKGIQTYTASNRTSLPGST
VTSSSSTFNDPASVDAHAYAQKVYDFYKSNFNRNSIDGNGLAIRSTTHYS
TRYNNAFWNGSQMVYGDGDGSQFIAFSGDLDVVGHELTHGVTEYTANLEY
YGQSGALNESISDIFGNTIEGKNWMVGDAIYTPGVSGDALRYMDDPTKGG
QPARMADYNNTSADNGGVHTNSGIPNKAYYLLAQGGTFGGVNVTGIGRSQ
AIQIVYRALTYYLTSTSNFSNYRSAMVQASTDLYGANSTQTTAVKNSLSA
VGIN (SEQ ID NO: 3)
PpoPro2 (Paenibacillus polymyxa, protein 2)
ATGTGKGVLGDTKSFTTTASGSSYQLKDTTRGNGIVTYTASNRQSIPGTL
LTDADNVWNDPAGVDAHAYAAKTYDYYKSKFGRDSVDGRGLQLRSTVHYG
SRYNNAFWNGSQMTYGDGDGSTFIAFSGDPDVVGHELTHGVTEYTSNLEY
YGESGALNEAFSDVIGNDIQRKNWLVGDDIYTPNIAGDALRSMSNPTLYD
QPDHYSNLYKGSSDNGGVHTNSGIINKAYYLLAQGGTFHGVAVNGIGRDA
AVQIYYSAFTNYLTSSSDFSNARAAVIQAAKDLYGANSAEATAAAKSFDA
VGVN (SEQ ID NO: 4)
PpoPro1 (Paenibacillus polymyxa, protein 1)
ATGTGKGVLGDSKSFTTTASGSSYQLKDTTRGNGIVTYTASNRQSIPGTI
LTDADNVWNDPAGVDAHAYAAKTYDYYKAKFGRNSIDGRGLQLRSTVHYG
SRYNNAFWNGSQMTYGDGDGSTFIAFSGDPDVVGHELTHGVTEYTSNLEY
YGESGALNEAFSDVIGNDIQRKNWLVGDDIYTPNIAGDALRSMSNPTLYD
QPDHYSNLYRGSSDNGGVHTNSGIINKAYYLLAQGGNFHGVTVNGIGRDA
AVQIYYSAFTNYLTSSSDFSNARAAVIQAAKDLYGANSAEATAAAKSFDA
VGVN (SEQ ID NO: 5)
PamPro1 (Paenibacillus amylolyticus, protein 1)
ATGTGTGVLGDTKTLTTTQSGSTFQLKDTTRGNGIQTYTANNGSSLPGSL
LTDSDNVWTDRAGVDAHAHAAATYDFYKNKFNRNGINGNGLLIRSTVHYG
SNYNNAFWNGAQIVFGDGDGTMFRSLSGDLDVVGHELTHGVIEYTANLEY
RNEPGALNEAFADIFGNTIQSKNWLLGDDIYTPNTPGDALRSLSNPTLYG
QPDKYSDRYTGSQDNGGVHINSGIINKAYFLAAQGGTHNGVTVTGIGRDK
AIQIFYSTLVNYLTPTSKFAAAKTATIQAAKDLYGATSAEATAITKAYQA
VGL (SEQ ID NO: 6)
PhuPro2 (Paenibacillus hunanensis, protein 2)
ATGSGTGVLGDNKTFQTTLSGSTYQLKDTTRGNGIYTYTASNRTTIPGTL
LTDADNVWTDGAAVDAHTYAGKVYDFYKTKFGRNSLDGNGLLIRSSVHYS
SRYNNAFWNGTQIVFGDGDGSTFIPLSGDLDVVGHELSHGVIEYTSNLQY
LNESGALNESYADVLGNSIQAKNWLIGDDVYTPGISGDALRSMSNPTLYG
QPDNYANRYTGSSDNGGVHTNSGITNKAFYLLAQGGTQNGVTVAGIGRDA
AVNIFYNTVAYYLTSTSNFAAAKNASIQAAKDLYGTGSSYVTSVTNAFRA
VGL (SEQ ID NO: 7)
PspPro2 (Paenibacillus sp., protein 2)
ATGTGRGVDGKTKSFTTTASGNRYQLKDTTRSNGIVTYTAGNRQTTPGTI
LTDTDNVWEDPAAVDAHAYAIKTYDYYKNKFGRDSIDGRGMQIRSTVHYG
KKYNNAFWNGSQMTYGDGDGSTFTFFSGDPDVVGHELTHGVTEFTSNLEY
YGESGALNEAFSDIIGNDIDGTSWLLGDGIYTPNIPGDALRSLSDPTRFG
QPDHYSNFYPDPNNDDEGGVHTNSGIINKAYYLLAQGGTSHGVTVTGIGR
EAAVFIYYNAFTNYLTSTSNFSNARAAVIQAAKDFYGADSLAVTSAIQSF
DAVGIK (SEQ ID NO: 8)
PspPro3 (Paenibacillus sp., protein 3)
ATGTGKGVLGDTKTFNTTASGSSYQLRDTTRGNGIVTYTASNRQSIPGTI
LTDADNVWNDPAGVDAHAYAAKTYDYYKEKFNRNSIDGRGLQLRSTVHYG
NRYNNAFWNGSQMTYGDGDGTTFIAFSGDPDVVGHELTHGVTEYTSNLEY
YGESGALNEAFSDIIGNDIQRKNWLVGDDIYTPRIAGDALRSMSNPTLYD
QPDHYSNLYRGSSDNGGVHTNSGIINKAYYLLAQGGTFHGVTVNGIGRDA
AVQIYYSAFTNYLTSSSDFSNARDAVVQAAKDLYGASSAQATAAAKSFDA
VGVN (SEQ ID NO: 9)
PpePro1 (Paenibacillus peoriae, protein 1)
ATGTGRGVDGVTKSFTTTASGNGYQLKDTTRSNGIVTYTANNRQTTPGTI
MTDADNVWNDPAAVDAHAYAIKTYDYYKNKFGRDSIDGRGMQIRSTVHYG
KKYVNAFWNGSQMTYGDGDGSTFTFFSGDPDVVGHELTHGVTEFTSNLEY
YGESGALNEAFSDIIGNDIDGANWLLGDGIYTPGIPGDALRSLSDPTRFG
QPDHYSNFYPDPNNDDEGGVHTNSGIINKAYYLLAQGGTSHGVKVTGIGR
EAAVFIYYNAFTNYLTSTSNFSNARAAVIQAAKDFYGADSLAVTSAIKSF
DAVGIK (SEQ ID NO: 10)
PtePro1 (Paenibacillus terrae, protein 1)
ATGTGVGVLGDTKTFTTTQSGTQYVNIQDTTRGGGIVTYSAGNTQSLPGT
LMRDTDNVWTDPAAVDAHAYAAVVYDYFKNNFNRDSLDGRGMAIKSTVHY
GSRYNNAFWNGTQIAYGDGDGTTFRAFSGDLDVIGHELTHGITEKTAGLI
YQGESGALNESISDVFGNTIQGKNWLIGDDIYTPSIPGDALRSMENPTLF
NQPDHYSNIYRGSDDNGGVHTNSGIPNKAFYLLAQGGTHRGVSVTGIGRG
DAAKIVYKALTYYLTSTSNFAAMRQAAISSATDLFGANSAQVNSVKAAYA
AVGI (SEQ ID NO: 11)
BbrProl (Brevibacillus brevis, protein 1)
VTATGKGVLGDTKQFETTKQGSTYMLKDTTRGKGIETYTANNRTSLPGTL
MTDSDNYWTDGAAVDAHAHAQKTYDYFRNVHNRNSYDGNGAVIRSTVHYS
TRYNNAFWNGSQMVYGDGDGTTFLPLSGGLDVVAHELTHAVTERTAGLVY
QNESGALNESMSDIFGAMVDNDDWLMGEDIYTPGRSGDALRSLQDPAAYG
DPDHYSKRYTGSQDNGGVHTNSGINNKAAYLLAEGGTHYGVRVNGIGRTD
TAKIYYHALTHYLTPYSNFSAMRRAAVLSATDLFGANSRQVQAVNAAYDA
VGVK (SEQ ID NO: 12)
1KEIA (Bacillus thermoptoteolyticus, thermolysin)
ITGTSTVGVGRGVLGDQKNINTTYSTYYYLQDNTRGNGIFTYDAKYRTTL
PGSLWADADNQFFASYDAPAVDAHYYAGVTYDYYKNVHNRLSYDGNNAAI
RSSVHYSQGYNNAFWNGSQMVYGDGDGQTFIPLSGGIDVVAHELTHAVTD
YTAGLIYQNESGAINEAISDIFGTLVEFYANKNPDWEIGEDVYTPGISGD
SLRSMSDPAKYGDPDHYSKRYTGTQDNGGVHINSGIINKAAYLISQGGTH
YGVSVVGIGRDKLGKIFYRALTQYLTPTSNFSQLRAAAVQSATDLYGSTS
QEVASVKQAFDAVGVK (SEQ ID NO: 13)
1NPCA (Bacillus cereus)
VTGTNKVGTGKGVLGDTKSLNTTLSGSSYYLQDNTRGATIFTYDAKNRST
LPGTLWADADNVFNAAYDAAAVDAHYYAGKTYDYYKATFNRNSINDAGAP
LKSTVHYGSNYNNAFWNGSQMVYGDGDGVTFTSLSGGIDVIGHELTHAVT
ENSSNLIYQNESGALNEAISDIFGTLVEFYDNRNPDWEIGEDIYTPGKAG
DALRSMSDPTKYGDPDHYSKRYTGSSDNGGVHTNSGIINKQAYLLANGGT
HYGVTVTGIGKDKLGAIYYRANTQYFTQSTTFSQARAGAVQAAADLYGAN
SAEVAAVKQ SFSAVGVN (SEQ ID NO: 14)
NprE_var (Bacillus subtilis)
AATTGTGTTLKGKTVSLNISSESGKYVLRDLSKPTGTQIITYDLQNREYN
LPGTLVSSTTNQFTTSSQRAAVDAHYNLGKVYDYFYQKFNRNSYDNKGGK
IVSSVHYGSRYNNAAWIGDQMIYGDGDGILFSPLSGSLDVTAHEMTHGVT
QETANLNYENQPGALNESFSDVFGYFNDTEDWDIGEDITISQPALRSLSN
PTKYGQPDNFKNYKNLPNTPAGDYGGVHTNSGIPNKAAYNTITKIGVNKA
EQIYYRALTVYLTPSSTFKDAKAALIQSARDLYGSQDAASVEAAWNAVGL
(SEQ ID NO: 15)
NprE (Bacillus subtilis)
AATTGTGTTLKGKTVSLNISSESGKYVLRDLSKPTGTQIITYDLQNREYN
LPGTLVSSTTNQFTTSSQRAAVDAHYNLGKVYDYFYQKFNRNSYDNKGGK
IVSSVHYGSRYNNAAWIGDQMIYGDGDGSFFSPLSGSMDVTAHEMTHGVT
QETANLNYENQPGALNESFSDVFGYFNDTEDWDIGEDITVSQPALRSLSN
PTKYGQPDNFKNYKNLPNTDAGDYGGVHTNSGIPNKAAYNTITKIGVNKA
EQIYYRALTVYLTPSSTFKDAKAALIQSARDLYGSQDAASVEAAWNAVGL
(SEQ ID NO: 16)

1. A metalloprotease polypeptide comprising a calcium binding region.

2. The polypeptide of claim 1, wherein the polypeptide comprises a modification in at least one amino acid residue in one of the calcium binding regions, Ca1-2, Ca3 and Ca4, (including residues 55-66, 136, 138, 177-190, and 193-200) of the polypeptide, wherein the amino acid positions of the polypeptide are numbered by correspondence with the amino acid sequence of Bacillus thermoproteolyticus metalloprotease set forth in SEQ ID NO: 13.

3. The polypeptide of claim 2, wherein the polypeptide comprises a modification in at least one amino acid residue in a calcium binding region 1-2 of residues 177-190, 136 and 138 of the polypeptide.

4. The polypeptide of any of the above claims, wherein the polypeptide comprises an amino acid at position 184 selected from the group consisting of lysine, threonine, alanine, glutamic acid and aspartic acid.

5. The polypeptide of any of the above claims, wherein the amino acid at position 185 is an amino acid other than aspartic acid.

6. The polypeptide of claim 5, wherein the amino acid at position 185 is a non-negatively charged residue.

7. The polypeptide of any of claim 5 or 6, wherein the amino acid at position 185 is a neutrally charged residue.

8. The polypeptide of any of claims 5-7, wherein the amino acid at position 185 is an asparagine or serine.

9. The polypeptide of any of the above claims, wherein the amino acid at position 187 is a non-negatively charged residue.

10. The polypeptide of claim 9, wherein the amino acid at position 187 is a neutrally charged residue.

11. The polypeptide of any of claim 9 or 10, wherein the amino acid at position 187 is a leucine or methionine.

12. The polypeptide of any of claims 1-8, wherein the amino acid at position 187 is an aspartic acid.

13. The polypeptide of any of the above claims, wherein the amino acid at position 188 is a leucine, valine or methionine.

14. The polypeptide of any of the above claims, wherein the amino acid at position 190 is a residue other than glutamic acid.

15. The polypeptide of claim 14, wherein the amino acid at position 190 is aspartic acid.

16. The polypeptide of any of the above claims, wherein the polypeptide comprises a deletion at amino acid residue positions 179-183.

17. The polypeptide of any of the above claims, wherein the amino acid at position 177 is a neutrally charged residue or aspartic acid.

18. The polypeptide of any of the above claims, wherein the amino acid at position 177 is glutamine or aspartic acid.

19. The polypeptide of any of the above claims, wherein the amino acid at position 178 is a residue selected from the group consisting of glycine, serine, arginine, alanine, asparagine, and threonine.

20. The polypeptide of any of the above claims, wherein the amino acid at position 136 is an aspartic acid or serine.

21. The polypeptide of any of the above claims, comprising a modification in at least one amino acid residue in a calcium binding region 3 of residues 55-66, wherein the amino acid positions of the polypeptide are numbered by correspondence with the amino acid sequence of Bacillus thermoproteolyticus metalloprotease set forth in SEQ ID NO: 13.

22. The polypeptide of any of the above claims, wherein the amino acid at position 55 is a residue selected from the group consisting of leucine, serine, valine, and methionine.

23. The polypeptide of any of the above claims, wherein the amino acid at position 56 is a residue selected from the group consisting of serine, arginine and threonine.

24. The polypeptide of any of the above claims, wherein the amino acid at position 57 is a serine.

25. The polypeptide of any of the above claims, wherein the amino acid at position 58 is a serine or threonine.

26. The polypeptide of any of the above claims, wherein the amino acid at position 59 is a residue selected from the group consisting of serine, threonine, and asparagine.

27. The polypeptide of any of the above claims, wherein the amino acid at position 60 is a serine.

28. The polypeptide of any of the above claims, wherein the amino acid at position 61 is a residue selected from the group consisting of isoleucine, valine, and threonine.

29. The polypeptide of any of the above claims, wherein the amino acid at position 62 is a residue selected from the group consisting of tryptophan and phenylalanine.

30. The polypeptide of any of the above claims, wherein the amino acid at position 63 is a residue selected from the group consisting of asparagine, glutamic acid, and threonine.

31. The polypeptide of any of the above claims, wherein the polypeptide comprises a deletion at amino acid residue positions 64-66.

32. The polypeptide of any of the above claims, comprising a modification in at least one amino acid residue in a calcium binding region 4 of residues 193-200, wherein the amino acid positions of the polypeptide are numbered by correspondence with the amino acid sequence of Bacillus thermoproteolyticus metalloprotease set forth in SEQ ID NO: 13.

33. The polypeptide of any of the above claims, wherein the amino acid at position 193 is a threonine.

34. The polypeptide of any of the above claims, wherein the amino acid at position 194 is an isoleucine.

35. The polypeptide of any of the above claims, wherein the amino acid at position 195 is a serine.

36. The polypeptide of any of the above claims, wherein the polypeptide comprises a deletion at amino acid residue positions 196-198.

37. The polypeptide of any of the above claims, wherein the amino acid at position 199 is a glutamine.

38. The polypeptide of any of the above claims, wherein the amino acid at position 200 is a proline.

39. The polypeptide of any of the above claims, wherein the calcium binding region 1-2 binds fewer than two calcium ions.

40. The polypeptide of any of the above claims, wherein the calcium binding region 3 binds fewer than one calcium ion.

41. The polypeptide of any of the above claims, wherein the calcium binding region 4 binds fewer than one calcium ion.

42. The polypeptide of any of the previous claims, wherein the polypeptide is a variant of a parent polypeptide.

43. The polypeptide of claim 42, wherein the variant comprises a modification in a calcium binding region of the parent polypeptide.

44. The polypeptide of claim 43, wherein the modification is to any of the amino acids listed in claims 4-38.

45. The polypeptide of any of the previous claims, wherein the parent polypeptide is an M4 metalloprotease.

46. The polypeptide of any of the previous claims, wherein the polypeptide has at least 60% sequence identity to the parent polypeptide.

47. The polypeptide of any of the previous claims, wherein the polypeptide has at least 60% sequence identity to any one of SEQ ID NOs: 1-15.

48. The polypeptide of any of the previous claims, wherein the polypeptide has at least 60% sequence identity to SEQ ID NOs: 13.

49. The polypeptide of any of the previous claims, wherein the polypeptide has metalloprotease activity.

50. A composition comprising any of the polypeptides of claims 1-49.

51. The composition of claim 50, wherein said composition is a cleaning composition.

52. The composition of claim 51, wherein said composition is a detergent composition.

53. The composition of claim 52, wherein said detergent composition is selected from the group consisting of a laundry detergent, a fabric softening detergent, a dishwashing detergent, and a hard-surface cleaning detergent.

54. The composition of any of claims 50 to 53, wherein said composition further comprises a surfactant.

55. The composition of claim 54, wherein said surfactant is selected from the group consisting of an anionic surfactant, a cationic surfactant, a zwitterionic surfactant, a ampholytic surfactant, a semi-polar non-ionic surfactant, and a combination thereof.

56. The composition of claim 55, wherein said surfactant is an ionic surfactant.

57. The composition of claim 55, wherein said surfactant is a non-ionic surfactant.

58. The composition of any of claims 50-57, wherein said composition further comprises at least one stabilizer.

59. The composition of any of claims 50-58, wherein said composition comprises from about 0.001 to about 10 weight % of said polypeptide.

60. The composition of any of claims 50-59, further comprising at least one bleaching agent.

61. The composition of any of claims 50-60, wherein said cleaning composition is phosphate-free.

62. The composition of any of claims 50-60, wherein said cleaning composition contains phosphate.

63. The composition of any of claims 50-62, further comprising at least one adjunct ingredient.

64. The composition of any of claims 50-63, wherein said composition is a granular, powder, solid, bar, liquid, tablet, gel, unit dose or paste composition.

65. The composition of any of claims 50-64, further comprising one or more additional enzymes or enzyme derivatives selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, and xylosidases, additional metallopotease enzymes and combinations thereof.

66. The composition of any of claims 50-65, wherein said composition is formulated at a pH of from about 5.0 to about 12.0.

67. A method of cleaning, comprising contacting a surface or an item with a composition comprising the variant of any one of claims 1-49.

68. A method of cleaning comprising contacting a surface or an item with the composition of any one of claims 50-66.

69. The method of claim 67 or 68, further comprising rinsing said surface or item after contacting said surface or item, respectively, with said composition.

70. The method of any one of claims 67-69, wherein said item is dishware.

71. The method of any one of claims 67-70, wherein said item is fabric.

72. The method of any one of claims 67-71, further comprising the step of rinsing said surface or item after contacting said surface or item with said composition.

73. The method of claim 72, further comprising the step of drying said surface or item after said rinsing of said surface or item.

74. A method of cleaning a surface or item, comprising: providing the composition of any of claims 50-66 and a surface or item in need of cleaning; and contacting said composition with said surface or item in need of cleaning under conditions suitable for the cleansing of said surface of said surface or item, to produce a cleansed surface or item.

75. The method of claim 74, further comprising the step of rinsing said cleansed surface or item to produce a rinsed surface or item.

76. The method of any of claim 74 or 75, further comprising the step of drying said rinsed surface or item.

77. A method for producing the variant of any of claims 1-49 comprising:
a. stably transforming a host cell with an expression vector comprising a polynucleotide encoding the variant of any of claims 1-49;
b. cultivating said transformed host cell under conditions suitable for said host cell to produce said protease; and
c. recovering said protease.

78. The method of claim 77, wherein said host cell is a filamentous fungus or bacterial cell.

79. The method of any of claim 77 or 78, wherein said host cell is selected from Bacillus spp., Streptomyces spp., Escherichia spp., Aspergillus spp., Trichoderma spp., Pseudomonas spp., Corynebacterium spp., Saccharomyces spp., or Pichia spp.

80. A textile processing composition comprising the variant of any one of claims 1-49.

81. An animal feed composition comprising the variant of any one of claims 1-49.

82. A leather processing composition comprising the variant of any one of claims 1-49.

83. A feather processing composition comprising the variant or recombinant polypeptide of any one of claims 1-49.

84. A lens cleaning composition comprising the variant of any one of claims 1-49.

85. A tissue debridement composition comprising the variant of any one of claims 1-49.

86. A tissue cell culture additive composition comprising the variant of any one of claims 1-49.


SEQUENCE LISTING (24 sequences)

SEQ ID NO: 1 - protein, 304 aa, Paenibacillus ehimensis
  misc_feature (1)..(304): PehPro1
ATGTGKGVLGDTKSFTTTQSGSTYQLKDTTRGQGIVTYSAGNRSSLPGTLLTSSSNIWNDGAAVDAHAYTAKVYDYYKNKFGRNSIDGNGFQLKSTVHYSSRYNNAFWNGVQMVYGDGDGVTFIPFSADPDVIGHELTHGVTEHTAGLEYYGESGALNESISDIIGNAIDGKNWLIGDLIYTPNTPGDALRSMENPKLYNQPDRYQDRYTGPSDNGGVHINSGINNKAFYLIAQGGTHYGVTVNGIGRDAAVQIFYDALINYLTPTSNFSAMRAAAIQAATDLYGANSSQVNAVKKAYTAVGVN

SEQ ID NO: 2 - protein, 303 aa, Paenibacillus barcinonensis
  misc_feature (1)..(303): PbaPro1
ATGTGTGVHGDTKTLTTTQSGSTYQLKDTTRGKGIQTYTANNRSSLPGSLSTSSNNVWTDRAAVDAHAYAAATYDFYKNKFNRNGIDGNGLLIRSTVHYGSNYKNAFWNGAQIVYGDGDGIEFGPFSGDLDVVGHELTHGVIEYTANLEYRNEPGALNEAFADIMGNTIESKNWLLGDGIYTPNIPGDALRSLSDPTLYNQPDKYSDRYTGSQDNGGVHINSGIINKAYYLAAQGGTHNGVTVSGIGRDKAVRIFYSTLVNYLTPTSKFAAAKTATIQAAKDLYGANSAEATAITKAYQAVGL

SEQ ID NO: 3 - protein, 304 aa, Paenibacillus hunanensis
  misc_feature (1)..(304): PhuPro1
ATGTGKGVLGDTKSFTVGTSGSSYVMTDSTRGKGIQTYTASNRTSLPGSTVTSSSSTFNDPASVDAHAYAQKVYDFYKSNFNRNSIDGNGLAIRSTTHYSTRYNNAFWNGSQMVYGDGDGSQFIAFSGDLDVVGHELTHGVTEYTANLEYYGQSGALNESISDIFGNTIEGKNWMVGDAIYTPGVSGDALRYMDDPTKGGQPARMADYNNTSADNGGVHTNSGIPNKAYYLLAQGGTFGGVNVTGIGRSQAIQIVYRALTYYLTSTSNFSNYRSAMVQASTDLYGANSTQTTAVKNSLSAVGIN

SEQ ID NO: 4 - protein, 304 aa, Paenibacillus polymyxa
  misc_feature (1)..(304): PpoPro2
ATGTGKGVLGDTKSFTTTASGSSYQLKDTTRGNGIVTYTASNRQSIPGTLLTDADNVWNDPAGVDAHAYAAKTYDYYKSKFGRDSVDGRGLQLRSTVHYGSRYNNAFWNGSQMTYGDGDGSTFIAFSGDPDVVGHELTHGVTEYTSNLEYYGESGALNEAFSDVIGNDIQRKNWLVGDDIYTPNIAGDALRSMSNPTLYDQPDHYSNLYKGSSDNGGVHTNSGIINKAYYLLAQGGTFHGVAVNGIGRDAAVQIYYSAFTNYLTSSSDFSNARAAVIQAAKDLYGANSAEATAAAKSFDAVGVN

SEQ ID NO: 5 - protein, 304 aa, Paenibacillus polymyxa
  misc_feature (1)..(304): PpoPro1
ATGTGKGVLGDSKSFTTTASGSSYQLKDTTRGNGIVTYTASNRQSIPGTILTDADNVWNDPAGVDAHAYAAKTYDYYKAKFGRNSIDGRGLQLRSTVHYGSRYNNAFWNGSQMTYGDGDGSTFIAFSGDPDVVGHELTHGVTEYTSNLEYYGESGALNEAFSDVIGNDIQRKNWLVGDDIYTPNIAGDALRSMSNPTLYDQPDHYSNLYRGSSDNGGVHTNSGIINKAYYLLAQGGNFHGVTVNGIGRDAAVQIYYSAFTNYLTSSSDFSNARAAVIQAAKDLYGANSAEATAAAKSFDAVGVN

SEQ ID NO: 6 - protein, 303 aa, Paenibacillus amylolyticus
  misc_feature (1)..(303): PamPro1
ATGTGTGVLGDTKTLTTTQSGSTFQLKDTTRGNGIQTYTANNGSSLPGSLLTDSDNVWTDRAGVDAHAHAAATYDFYKNKFNRNGINGNGLLIRSTVHYGSNYNNAFWNGAQIVFGDGDGTMFRSLSGDLDVVGHELTHGVIEYTANLEYRNEPGALNEAFADIFGNTIQSKNWLLGDDIYTPNTPGDALRSLSNPTLYGQPDKYSDRYTGSQDNGGVHINSGIINKAYFLAAQGGTHNGVTVTGIGRDKAIQIFYSTLVNYLTPTSKFAAAKTATIQAAKDLYGATSAEATAITKAYQAVGL

SEQ ID NO: 7 - protein, 303 aa, Paenibacillus hunanensis
  misc_feature (1)..(303): PhuPro2
ATGSGTGVLGDNKTFQTTLSGSTYQLKDTTRGNGIYTYTASNRTTIPGTLLTDADNVWTDGAAVDAHTYAGKVYDFYKTKFGRNSLDGNGLLIRSSVHYSSRYNNAFWNGTQIVFGDGDGSTFIPLSGDLDVVGHELSHGVIEYTSNLQYLNESGALNESYADVLGNSIQAKNWLIGDDVYTPGISGDALRSMSNPTLYGQPDNYANRYTGSSDNGGVHTNSGITNKAFYLLAQGGTQNGVTVAGIGRDAAVNIFYNTVAYYLTSTSNFAAAKNASIQAAKDLYGTGSSYVTSVTNAFRAVGL

SEQ ID NO: 8 - protein, 306 aa, Paenibacillus  sp.
  misc_feature (1)..(306): PspPro2
ATGTGRGVDGKTKSFTTTASGNRYQLKDTTRSNGIVTYTAGNRQTTPGTILTDTDNVWEDPAAVDAHAYAIKTYDYYKNKFGRDSIDGRGMQIRSTVHYGKKYNNAFWNGSQMTYGDGDGSTFTFFSGDPDVVGHELTHGVTEFTSNLEYYGESGALNEAFSDIIGNDIDGTSWLLGDGIYTPNIPGDALRSLSDPTRFGQPDHYSNFYPDPNNDDEGGVHTNSGIINKAYYLLAQGGTSHGVTVTGIGREAAVFIYYNAFTNYLTSTSNFSNARAAVIQAAKDFYGADSLAVTSAIQSFDAVGIK

SEQ ID NO: 9 - protein, 304 aa, Paenibacillus  sp.
  misc_feature (1)..(304): PspPro3
ATGTGKGVLGDTKTFNTTASGSSYQLRDTTRGNGIVTYTASNRQSIPGTILTDADNVWNDPAGVDAHAYAAKTYDYYKEKFNRNSIDGRGLQLRSTVHYGNRYNNAFWNGSQMTYGDGDGTTFIAFSGDPDVVGHELTHGVTEYTSNLEYYGESGALNEAFSDIIGNDIQRKNWLVGDDIYTPRIAGDALRSMSNPTLYDQPDHYSNLYRGSSDNGGVHTNSGIINKAYYLLAQGGTFHGVTVNGIGRDAAVQIYYSAFTNYLTSSSDFSNARDAVVQAAKDLYGASSAQATAAAKSFDAVGVN

SEQ ID NO: 10 - protein, 306 aa, Paenibacillus peoriae
  misc_feature (1)..(306): PpePro1
ATGTGRGVDGVTKSFTTTASGNGYQLKDTTRSNGIVTYTANNRQTTPGTIMTDADNVWNDPAAVDAHAYAIKTYDYYKNKFGRDSIDGRGMQIRSTVHYGKKYVNAFWNGSQMTYGDGDGSTFTFFSGDPDVVGHELTHGVTEFTSNLEYYGESGALNEAFSDIIGNDIDGANWLLGDGIYTPGIPGDALRSLSDPTRFGQPDHYSNFYPDPNNDDEGGVHTNSGIINKAYYLLAQGGTSHGVKVTGIGREAAVFIYYNAFTNYLTSTSNFSNARAAVIQAAKDFYGADSLAVTSAIKSFDAVGIK

SEQ ID NO: 11 - protein, 303 aa, Paenibacillus terrae
  misc_feature (1)..(303): PtePro1
ATGTGVGVLGDTKTFTTTQSGTQYVMQDTTRGGGIVTYSAGNTQSLPGTLMRDTDNVWTDPAAVDAHAYAAVVYDYFKNNFNRDSLDGRGMAIKSTVHYGSRYNNAFWNGTQIAYGDGDGTTFRAFSGDLDVIGHELTHGITEKTAGLIYQGESGALNESISDVFGNTIQGKNWLIGDDIYTPSIPGDALRSMENPTLFNQPDHYSNIYRGSDDNGGVHTNSGIPNKAFYLLAQGGTHRGVSVTGIGRGDAAKIVYKALTYYLTSTSNFAAMRQAAISSATDLFGANSAQVNSVKAAYAAVGI

SEQ ID NO: 12 - protein, 304 aa, Brevibacillus brevis
  misc_feature (1)..(304): BbrPro1
VTATGKGVLGDTKQFETTKQGSTYMLKDTTRGKGIETYTANNRTSLPGTLMTDSDNYWTDGAAVDAHAHAQKTYDYFRNVHNRNSYDGNGAVIRSTVHYSTRYNNAFWNGSQMVYGDGDGTTFLPLSGGLDVVAHELTHAVTERTAGLVYQNESGALNESMSDIFGAMVDNDDWLMGEDIYTPGRSGDALRSLQDPAAYGDPDHYSKRYTGSQDNGGVHTNSGINNKAAYLLAEGGTHYGVRVNGIGRTDTAKIYYHALTHYLTPYSNFSAMRRAAVLSATDLFGANSRQVQAVNAAYDAVGVK

SEQ ID NO: 13 - protein, 316 aa, Bacillus thermoproteolyticus
  misc_feature (1)..(316): 1KEI.A,  thermolysin
ITGTSTVGVGRGVLGDQKNINTTYSTYYYLQDNTRGNGIFTYDAKYRTTLPGSLWADADNQFFASYDAPAVDAHYYAGVTYDYYKNVHNRLSYDGNNAAIRSSVHYSQGYNNAFWNGSQMVYGDGDGQTFIPLSGGIDVVAHELTHAVTDYTAGLIYQNESGAINEAISDIFGTLVEFYANKNPDWEIGEDVYTPGISGDSLRSMSDPAKYGDPDHYSKRYTGTQDNGGVHINSGIINKAAYLISQGGTHYGVSVVGIGRDKLGKIFYRALTQYLTPTSNFSQLRAAAVQSATDLYGSTSQEVASVKQAFDAVGVK

SEQ ID NO: 14 - protein, 317 aa, Bacillus cereus
  misc_feature (1)..(317): 1NPC.A
VTGTNKVGTGKGVLGDTKSLNTTLSGSSYYLQDNTRGATIFTYDAKNRSTLPGTLWADADNVFNAAYDAAAVDAHYYAGKTYDYYKATFNRNSINDAGAPLKSTVHYGSNYNNAFWNGSQMVYGDGDGVTFTSLSGGIDVIGHELTHAVTENSSNLIYQNESGALNEAISDIFGTLVEFYDNRNPDWEIGEDIYTPGKAGDALRSMSDPTKYGDPDHYSKRYTGSSDNGGVHTNSGIINKQAYLLANGGTHYGVTVTGIGKDKLGAIYYRANTQYFTQSTTFSQARAGAVQAAADLYGANSAEVAAVKQSFSAVGVN

SEQ ID NO: 15 - protein, 300 aa, Bacillus subtilis
  misc_feature (1)..(300): NprE_var
AATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGILFSPLSGSLDVTAHEMTHGVTQETANLNYENQPGALNESFSDVFGYFNDTEDWDIGEDITISQPALRSLSNPTKYGQPDNFKNYKNLPNTPAGDYGGVHTNSGIPNKAAYNTITKIGVNKAEQIYYRALTVYLTPSSTFKDAKAALIQSARDLYGSQDAASVEAAWNAVGL

SEQ ID NO: 16 - protein, 300 aa, Bacillus subtilis
  misc_feature (1)..(300): NprE
AATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 17 - protein, 8 aa, Bacillus thermoproteolyticus
YTPGISGD

SEQ ID NO: 18 - protein, 5 aa, Bacillus subtilis
TISQP

SEQ ID NO: 19 - protein, 12 aa, Bacillus thermoproteolyticus
WADADNQFFASY

SEQ ID NO: 20 - protein, 12 aa, Bacillus thermoproteolyticus
WASSSNQFFASY

SEQ ID NO: 21 - protein, 9 aa, Paenibacillus ehimensis
LTSSSNIWN

SEQ ID NO: 22 - protein, 9 aa, Bacillus thermoproteolyticus
EFYANKNPD

SEQ ID NO: 23 - protein, 4 aa, Paenibacillus ehimensis
DGKN

SEQ ID NO: 24 - protein, 4 aa, Artificial Sequence
  Synthetic peptide
  MOD_RES (1)..(1): Succinyl- at 5′ end
  MOD_RES (4)..(4): p-nitroanilide at 3′ end
AAPF